(19) United States
(12) Patent Application Publication
(10) Pub. No.: US 2025/0188154 A1
(43) Pub. Date: Jun. 12, 2025

(54) CORONAVIRUS ANTIBODIES AND USES THEREOF

(71) Applicants: Duke University, Durham, NC (US); THE UNITED STATES OF AMERICA, AS REPRESENTED BY THE SECRETARY, DEPARTMENT OF HEALTH & HUMAN SERVICES, Bethesda, MD (US)

(72) Inventors: Gregory Sempowski, Durham, NC (US); Barton F. Haynes, Durham, NC (US); Kevin Saunders, Durham, NC (US); Dapeng Li, Durham, NC (US); Xiaozhi Lu, Durham, NC (US); Robert J. Edwards, Durham, NC (US); Priyamvada Acharya, Durham, NC (US); Kartik Manne, Durham, NC (US); Sophie Gobeil, Durham, NC (US); John R. Mascola, Bethesda, MD (US); Barney S. Graham, Bethesda, MD (US); Tongqing Zhou, Bethesda, MD (US)

(21) Appl. No.: 18/026,544

(22) PCT Filed: Sep. 15, 2021
(86) PCT No.: PCT/US2021/050552

§ 371 Date: Mar. 15, 2023

Related U.S. Application Data
(60) Provisional application No. 63/078,666, filed on Sep. 15, 2020.
(60) Provisional application No. 63/126,267, filed on Dec. 16, 2020.
(60) Provisional application No. 63/180,564, filed on Apr. 27, 2021.
(60) Provisional application No. 63/212,078, filed on Jun. 17, 2021.

(51) Int. Cl. C07K 16/10; A61K 39/00; A61P 37/04

(52) U.S. Cl. CPC C07K 16/1003; A61P 37/04; A61K 2039/505; C07K 2317/24; C07K 2317/55; C07K 2317/76; C07K 2317/92

(57) ABSTRACT

Recombinant monoclonal antibodies (mAbs) and fragments that bind specifically to coronavirus spike protein. Herein, monoclonal antibodies were recombinantly derived from isolated B cell from coronavirus infected individuals. Such antibodies bind various epitopes on the coronavirus spike protein and are neutralizing. The invention provides methods for using the inventive antibodies in prophylactic and/or therapeutic methods to prevent or treat coronavirus infection.

This application claims the benefit of and priority to U.S. Patent Application No. 63/078,666 filed on Sep. 15, 2020, U.S. Patent Application No. 63/126,267 filed on Dec. 16, 2020, U.S. Patent Application No. 63/180,564 filed on Apr. 27, 2021, and U.S. Patent Application No. 63/212,078 filed on Jun. 17, 2021 the content of each application is herein incorporated by reference in its entirety.

### GOVERNMENT SUPPORT

This invention was made with government support under HR0011-17-2-0069 awarded by Defense Advanced Research Projects Agency (DARPA), U19AI142596 awarded by National Institutes of Health: National Institute of Allergy and Infectious Diseases (NIAID), and a contract from the State of North Carolina funded by the Coronavirus Aid, Relief, and Economic Security Act (CARES Act). The government has certain rights in the invention.

### ASCII TABLES AND SEQUENCE LISTING FILES

The instant application contains a Sequence Listing which has been submitted electronically in ASCII format and is hereby incorporated by reference in its entirety. Said ASCII copy, created Sep. 14, 2021, is named 2933311-000042-WO1 SL.txt and is 962,359 bytes in size.

This patent application incorporates by reference herein the tables that have been submitted electronically in ASCII format in U.S. Patent Application No. 63/126,267 filed on Dec. 16, 2020, U.S. Patent Application No. 63/180,564 filed on Apr. 27, 2021, and U.S. Patent Application No. 63/212,078 filed on Jun. 17, 2021. Said ASCII tables, created Nov. 30, 2020, are as follows:
(1) Table S1-SARS-COV-1 Donor MBC.txt, 93,910 bytes
(2) Table S1-SARS-COV-2 Donor MBC.txt, 532,698 bytes
(3) Table S1-SARS-COV-2 Donor Plasmablast.txt, 257,140 bytes
(4) Table S8-COVID-19 NTD NAbs (n=10).txt, 5,652 bytes
(5) Table S8-COVID-19 NTD non-Abs (n=31).txt, 15,760 bytes
(6) Table S8-COVID-19 RBD NAbs (n=44).txt, 22,497 bytes
(7) Table S8-COVID-19 RBD non-NAbs (n=37).txt, 18,002 bytes
(8) Table S8-COVID-19 S2 Abs (n=65).txt, 33,848 bytes
(9) Table S8-Cross-reactive Abs (n=80).txt, 55,691 bytes

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### TECHNICAL FIELD

The invention relates to human antibodies binding to and/or neutralizing Coronaviruses, including but not limited to SARS Coronavirus 2 (SARS-COV-2) virus and their uses.

### BACKGROUND

Severe Acute Respiratory Syndrome Coronavirus 2 (SARS-COV-2) is related to SARS-CoV and several SARS-like bat CoVs (F. Wu, et al., A new coronavirus associated with human respiratory disease in China. Nature 579, 265-269 (2020)). CoV entry into host cells is mediated by the viral S glycoprotein, which forms trimeric spikes on the viral surface (F. Li, Structure, function, and evolution of coronavirus spike proteins. Annu. Rev. Virol. 3, 237-261 (2016)). Each monomer in the trimeric S assembly is a heterodimer of S1 and S2 subunits. The S1 subunit is composed of four domains: an N-terminal domain (NTD), a C-terminal domain (CTD), and subdomains I and II (A. C. Walls, et al., Cryo-electron microscopy structure of a coronavirus spike glycoprotein trimer. Nature 531, 114-117 (2016); M. A. Tortorici, D. Veesler, Structural insights into coronavirus entry. Adv. Virus Res. 105, 93-116 (2019); D. Wrapp, et al., Cryo-EM structure of the 2019-nCOV spike in the prefusion conformation. Science 367, 1260-1263 (2020)). The CTD of both SARS-COV and SARS-COV-2 functions as the receptor-binding domain (RBD) for the shared entry receptor, human angiotensin converting enzyme 2 (hACE2) (W. Song et al., Cryo-EM structure of the SARS coronavirus spike glycoprotein in complex with its host cell receptor ACE2. PLOS Pathog. 14, e1007236 (2018); J. Lan et al., Structure of the SARS-COV-2 spike receptor-binding domain bound to the ACE2 receptor. Nature 581, 215-220 (2020); M. Hoffmann et al., SARS-COV-2 cell entry depends on ACE2 and TMPRSS2 and is blocked by a clinically proven protease inhibitor. Cell 181, 271-280.e8 (2020); Q. Wang et al., Structural and functional basis of SARS-COV-2 entry by using human ACE2. Cell 181, 894-904.e9 (2020); F. Li, Receptor recognition mechanisms of coronaviruses: A decade of structural studies. J. Virol. 89, 1954-1964 (2015)). The S2 subunit contains the fusion peptide, heptad repeat 1 and 2, and a transmembrane domain, all of which are required for fusion of the viral and host cell membranes.

The S glycoprotein of HCoVs is the primary target for neutralizing antibodies (nAbs) (S. Jiang, et al., Neutralizing antibodies against SARS-COV-2 and other human coronaviruses. Trends Immunol. 41, 355-359 (2020)). SARS-COV and SARS-COV-2 share 76% amino acid identity in their S proteins, raising the possibility of conserved immunogenic surfaces on these antigens. Studies of convalescent sera and a limited number of monoclonal antibodies (mAbs) have revealed limited to no cross-neutralizing activity, demonstrating that conserved antigenic sites are rarely targeted by nAbs (D. Wrapp, et al., Cryo-EM structure of the 2019-nCOV spike in the prefusion conformation. Science 367, 1260-1263 (2020); Q. Wang et al., Structural and functional basis of SARS-COV-2 entry by using human ACE2. Cell 181, 894-904.e9 (2020); X. Ou et al., Characterization of spike glycoprotein of SARS-COV-2 on virus entry and its immune cross-reactivity with SARS-COV. Nat. Commun. 11, 1620 (2020); H. Lv et al., Cross-reactive antibody response between SARS-COV-2 and SARS-COV infections. Cell Rep. 31, 107725 (2020)). However, the frequencies, specificities, and functional activities of cross-reactive antibodies induced by natural SARS-COV and SARS-COV-2 infection remain poorly defined.

### SUMMARY OF THE INVENTION

In certain aspects the present invention provides monoclonal antibodies (mAbs) and fragments thereof that bind to alpha, beta, delta or gamma coronavirus antigens, including without limitation SARS-COV-2 antigens. In certain embodiments, these are neutralizing antibodies that bind to SARS-COV-2 spike protein. In other embodiments, these are antibody dependent cellular cytotoxicity or other anti-viral Fc-Receptor mediating antibodies. As used herein, the term “antibody” is used broadly, and can refer to proteins and/or nucleic acids of a full-length antibody, a fragment, or synthetic forms thereof.

In certain aspects the present invention provides recombinant coronavirus antibodies, or antigen binding fragments thereof, wherein in certain non-limiting embodiments the antibody or fragment thereof binds to a coronavirus. A recombinant coronavirus monoclonal antibody, or an antigen binding fragment thereof, which binds to coronavirus spike protein and comprises a variable heavy (VH) domain and a variable light (VL) domain that have amino acid sequences that have an overall 80%, 81%, 82%, 83%, 84%, 85%, 86%, 87%, 88%, 89%, 90%, 91%, 92%, 93%, 94%, 95%, 95%, 96%, 97%, 98%, 99% or 100% sequence identity to the VH and VL domains of an antibody described herein, for example listed in Tables 3-6, 10 or 11, or wherein the VH domain and VL domain each have at least 80%, 81%, 82%, 83%, 84%, 85%, 86%, 87%, 88%, 89%, 90%, 91%, 92%, 93%, 94%, 95%, 95%, 96%, 97%, 98%, 99% or 100% sequence identity to the VH and VL domains, respectively, of an antibody an antibody described herein, for example listed in Tables 3-6, 10 or 11. In certain embodiments the antibodies are neutralizing antibodies. In certain embodiments, the antibody specifically binds to RBD of the coronavirus spike protein. In certain embodiments, the RBD binding antibodies block ACE2 receptor interaction. In certain embodiments, the antibody specifically binds to NTD of the coronavirus spike protein. In certain embodiments, the NTD antibodies do not display ADE. In certain embodiments, the antibody is specific for CoV2. In certain embodiments the antibody is cross-reactive with other coronaviruses, e.g. without limitation SARS-COV-1, MERS-COV, 229E, NL63, HKUI, OC43, bat coronavirus, and/or pangolin coronavirus. In certain embodiments, the antibody, or the antigen-binding fragment thereof, wherein the concentration of the antibody, or antigen-binding fragment thereof, required for 50% neutralization of coronavirus, e.g but not limited to CoV2 virus, (IC50) is as described in Table 4A and 4B. In certain embodiments, IC50 is up to about 1 mcg/ml, up to about 500 ng/ml, up to about 250 ng/ml, up to about 100 ng/ml or up to about 50 ng/ml. In certain embodiments, the antibody or antigen binding fragment thereof, binds to the surface of coronavirus-infected cells and mediates either clearance, complement lysis or NK or CD8 killing of coronavirus-infected cells.

There are additional antibodies and sequences described in FIGS. 38, 45 and 46. All embodiments described herein that refer to Table 3, 4, 5, 6, 10 or 11 may also encompass these additional human antibody sequences.

In certain aspects, the antibody binds to CoV-2 spike protein. In certain aspects, the antibody binds to the CoV-2 spike protein and may not be cross-reactive to SARS-COV spike protein. In certain aspects, the antibody binds CoV-2 spike protein and is cross-reactive to other human or animal-derived coronavirus spike proteins, for example but not limited to SARS-COV-1 spike protein. In certain aspects, the antibody can neutralize or inhibit binding of SARS COV-2 to the human or animal ACE2 receptor. In certain aspects, the antibody has a binding affinity to SARS-COV-2 spike protein that is stronger than the binding affinity between SARS-COV-2 spike protein and the human ACE2 receptor. Herein, monoclonal antibodies were recombinantly produced from B cell receptor cells isolated from patients infected with SARS-COV2. The invention provides methods for using such antibodies for passive immunotherapy and/or diagnostic purposes.

In certain aspects, provided are antibodies and fragments comprising VH and VL (as used herein, VH and VL can also be referred to as VH or VL and VH or VL, respectively) sequences of the antibodies described in Tables 3-6, 10 or 11, the Examples and Figures disclosing amino acid and nucleic acid sequences. Figures disclose complete heavy and light nucleotide sequences, and the VH and VL domains are readily determined in these sequences. For example, VH and VL are readily derived from the nucleotide sequences or amino acid sequences by any suitable method, such as but not limited by IMGT and other online tools as cited herein, which tools not only provide amino acid translations, but also variable domains of the heavy and light chains, predicted CDR and framework boundaries. See, e.g., http://www.imgt.org/IMGT_vquest/. More specifically, for example, a nucleotide sequence for a VH or VL domain can be input at http://www.imgt.org/IMGT_vquest/input and results include “V-REGION translation” that provides the nucleotide sequence and amino acid translation along with the framework and CDR boundaries according to the IMGT scheme.

In certain aspects, the antibodies or fragments have a binding specificity as described in Tables 3-6, 10 or 11, the accompanying Examples and Figures.

In certain aspects, the antibodies are recombinant antibodies having an IgG or IgM Fc domain, or a portion thereof.

In certain aspects, provided are recombinant antibodies and fragments comprising HCDR1-3 and LCDR1-3 (as used herein, the VH CDRs can be referred to as HCDR1-3 or CDRH1-3; likewise, the VL CDRs can be referred to as LCDR1-3 or CDRL1-3) from the pairs of VH and VL sequences as described in Table 3-6, 10 or 11, and accompanying Figures. In one aspect, the antibody comprises HCDR1-3 and LCDR1-3 of antibody DH1043. In one aspect, the antibody comprises HCDR1-3 and LCDR1-3 of antibody DH1042. In one aspect, the antibody comprises HCDR1-3 and LCDR1-3 of antibody DH1041. In one aspect, the antibody comprises HCDR1-3 and LCDR1-3 of antibody DH1047. In one aspect, the antibody comprises HCDR1-3 and LCDR1-3 of antibody DH1050.1.

In certain aspects, an antibody that comprises HCDR1-3 and LCDR1-3 of an antibody of Table 3-6, 10 or 11 is affinity matured by testing mutations in one or more of the CDRs. In one aspect, an antibody that comprises HCDR1-3 and LCDR1-3 of an antibody of Tables 3-6, 10 or 11 or is affinity matured by testing mutations only in HCDR3. In certain aspects, mutations are favorable when the antibody maintains binding specificity but improves affinity or avidity for the antigen.

In certain aspects, the invention provides a pharmaceutical composition comprising the recombinant antibodies of the invention.

In certain aspects, the invention provides nucleic acids comprising sequences encoding antibodies comprising VH and VL sequences of the inventive antibodies, e.g. from Tables 3-6, 10 or 11. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′cap.

In certain aspects, the invention provides a kit comprising: a composition comprising an antibody of the invention, a syringe, needle, or applicator for administration of the antibody to a subject; and instructions for use.

In certain aspects, the invention provides prophylactic methods comprising administering the pharmaceutical composition of the invention.

In certain aspects, the invention provides methods of treatment comprising administering the pharmaceutical composition of the invention. The methods are applicable to diseases or conditions, e.g. but not limited to prophylaxis, suspected or diagnosed coronavirus infection, that will benefit from passive immunization with coronavirus targeting antibodies or combinations thereof.

In certain aspects, provided are antibodies and fragments comprising VH and VL sequences as described, e.g. Tables 3-6, 10 or 11.

In certain aspects, the invention provides a pharmaceutical composition comprising the recombinant antibodies of the invention.

In certain aspects, the invention provides nucleic acids comprising sequences encoding coronavirus antibodies comprising VL and VH sequences of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′cap.

In certain aspects, the invention provides prophylactic methods comprising administering the pharmaceutical composition of the invention. In certain embodiments, the methods lead to protection from infection, disease, reduced severity of disease, including but not limited to reduced severity of symptoms and/or reduced duration of coronavirus infection and disease.

In non-limiting embodiments, the coronavirus infection is caused by SARS-COV-2. In non-limiting embodiments the disease is COVID19.

In certain aspects, the invention provides methods of treatment comprising administering the pharmaceutical composition of the invention. The pharmaceutical composition comprises at least one antibody of the invention formulated as a recombinant protein, a nucleic acid encoding the antibody or a combination thereof.

In certain embodiments, the methods comprise administering additional therapeutic or prophylactic agents, including but not limited to additional coronavirus neutralizing antibodies, small molecule therapeutics, or any other suitable agent. In certain embodiments, the coronavirus antibodies have different specificities.

In certain aspects, the invention provides a kit comprising: a composition comprising an antibody of the invention, a syringe, needle, or applicator for administration of the antibody to a subject; and instructions for use.

In certain aspects the invention provides a method of treating a subject, the method comprising steps of: administering to a subject suffering from or susceptible to coronavirus infection therapeutically effective amount of an antibody of the invention. In certain embodiments the antibody is administered as a therapeutic and/or prophylactic measure. Prophylactic methods comprise pre-exposure administration. In some embodiments, the methods comprise administering an antibody of the invention as recombinant protein. In some embodiments, the methods comprise administering an antibody of the invention as a nucleic acid.

In some embodiments, the methods comprise administering a combination treatment with antibodies, wherein the combination comprises at least one inventive antibody. In some embodiments, at least one of the antibodies in a combination treatment is a neutralizing antibody. Without wishing to be bound by theory, a treatment method comprising a combination of antibodies targeting different epitopes can reduce viral neutralization escape.

In some embodiments, at least one or more of the antibodies has RBD specificity. In some embodiments, at least one or more of the antibodies is RBD/ACE2 blocking. In some embodiments, where for example two antibodies are administered, the antibodies have different epitopes.

Different epitopes can be located in the same area of the spike protein or different area of the spike protein, e.g. but not limited to the RBD, NTD, S2 etc.

Several of the antibodies do not cross-block each other in experiment of surface plasmon resonance assays. Table 8 shows the RBD antibodies that do or do not cross-block each other, Table 9 shows the NTD antibodies that do or do not block each other and Table 10 shows the RBD and NTD antibodies that do not block each other. Those antibodies that do not block each other can be used in combinations of neutralizing antibodies for either prevention of SARS-COV-2 infection or for treatment of COVID-1 disease. Other antibodies listed in Tables 3-6, 10 or 11 that are not in the Tables 8-10 herein, can also be combined for prevention or treatment by performing the same types of blocking experiments with surface plasmon resonance.

In certain embodiments, the antibody used in the methods of the invention is any one of the antibodies from Table 3-6, 10 or 11. In certain embodiments, the antibody is any one of Ab026116 (DH1043), or Ab026124 (DH1041) or Ab026103 (DH1042) (RBD/ACE2 blocking Abs), or a combination thereof. In certain embodiments, the methods comprise administering a combination of antibodies, wherein at least one of the antibodies in a combination is selected from Table 3-6, 10 or 11. In some embodiments the combination comprises RBD/ACE2 blocking antibody, e.g. Ab026116 (DH1043, RBD/ACE2 blocking), Ab026124 (DH1041) or Ab026103 (DH1042), Ab026204 (DH1046) (RBD/SARS1-CoV-1 cross-reactive), and any one of Ab026016 (DH1050.1), Ab12213 (DH1050.2) or Ab026013 (DH1051) (NTD), or a combination thereof. SPR competition experiments with DH1047, DH1046, DH1073 and DH1235 show that DH1047, DH1046, and DH1235 were outcompeted by one another (FIG. 74D), whereas DH1073 was not, indicating that DH1073 targets a distinct non-cross-competing epitope. Thus, in certain non-limiting embodiments, RBD antibodies targeting non-cross-competing epitopes can be used in a combination treatment. In non-limiting embodiments, the combination comprises DH1073 antibody or antigen binding fragment thereof, and any other non-cross-competing antibody, e.g. without limitation DH1047, DH1046, or DH1042. These non-crossreactive antibodies targeting RBD can be combined with an antibody targeting any other epitope, e.g., without limitation NTD targeting antibodies.

In certain embodiments, the antibody or antigen binding fragment preferentially or specifically binds to coronavirus spike protein. In certain embodiments, the VH domain and VL domain each have at least 90% sequence identity to the VH and VL domains, respectively, of an antibody listed in Tables 3-6, 10 or 11.

In certain embodiments, (a) VL domain CDRL1-3 regions together have no more than 10 amino acid variations as compared to the corresponding CDRL1-3 regions of an antibody listed in Tables 3-6, 10 or 11, and (b) VH domain CDRH1-3 regions together have no more than 10 amino acid variations as compared to the corresponding CDRH1-3 regions of an antibody listed in Tables 3-6, 10 or 11.

In certain embodiments, wherein the antibody or fragment is human or fully-human.

In certain embodiments, wherein the VH domain and VL domain of the antibody or fragment comprises framework regions that each have no more than 20, or 10 or 5 amino acid variations as compared to the corresponding framework regions of a human antibody listed in Tables 3-6, 10 or 11.

In certain embodiments, (a) VL domain CDRL1-3 regions together have no more than 10 amino acid variations as compared to the corresponding CDRL1-3 regions of an antibody listed in Tables 3-6, 10 or 11, (b) VH domain CDRH1-3 regions together have no more than 10 amino acid variations as compared to the corresponding CDRH1-3 regions of the antibody listed in Tables 3-6, 10 or 11, and (c) the VL domain and VH domain framework regions each have no more than 10 or 5 amino acid variations as compared to the corresponding framework regions of the human antibody listed in Tables 3-6, 10 or 11.

In certain embodiments, the antibody, or the antigen-binding fragment thereof comprises an Fc moiety. In certain embodiments, wherein the antibody, or antigen-binding fragment thereof, comprises a mutation(s) in the Fc moiety that reduces binding of the antibody to an Fc receptor and/or increases the half-life of the antibody.

In certain embodiments, the antibody, or the antigen binding fragment thereof, is a purified antibody, a single chain antibody, Fab, Fab′, F(ab′)2, Fv or scFv.

In certain embodiments, the antibody is of any isotype.

In certain aspect the invention provide antibody, or the antigen-binding fragment thereof, for use as a medicament.

In certain aspect the invention provide a nucleic acid molecule comprising a polynucleotide encoding an antibody of the invention or the antigen-binding fragment thereof.

In certain embodiments, the polynucleotide sequence comprises, consists essentially of or consists of a nucleic acid sequence according to any one of the sequences in FIG. 20-25, 46; or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional variation is 80%, 81%, 82%, 83%, 84%, 85%, 86%, 87%, 88%, 89%, 90%, 91%, 92%, 93%, 94%, 95%, 96%, 97%, 98% or 99% identity.

In certain aspects the invention provides a vector comprising a nucleic acid molecule encoding an antibody of the invention or antigen binding fragment thereof.

In certain aspects the invention provides a cell expressing an antibody of the invention, or the antigen binding fragment thereof.

In certain aspects the invention provides pharmaceutical composition comprising an antibody of the invention, a combination of antibodies comprising at least one antibody of the invention or the antigen binding fragment thereof, a nucleic acid encoding an antibody of the invention or a fragment thereof and/or a cell expressing an antibody of the invention, or the antigen binding fragment thereof, and optionally a pharmaceutically acceptable carrier. In certain embodiments, the composition is suitable for pharmaceutical use.

In certain aspect, the composition further comprises a pharmaceutically acceptable excipient, diluent or carrier.

In certain aspects, the invention provides an in vitro transcription system to synthesize ribonucleic acids (RNAs) encoding antibodies of the invention, comprising: a reaction vessel, a DNA vector template comprising nucleic acid sequence encoding an antibody of the invention as described in Tables 3-6, 10 or 11, and reagents for carrying out an in vitro transcription reaction that produces mRNA encoding an antibody or fragment thereof of the invention. In certain embodiments the mRNA is modified mRNA.

In certain aspects, the invention provides a method for manufacturing an mRNA encoding an antibody or antigen binding fragment thereof, comprising:
a. providing an in vitro transcription reaction vessel comprising a DNA template encoding an antibody or fragment thereof according to any of the preceding claims and reagents under conditions suitable for in vitro transcription of the nucleic acid template, thereby producing an mRNA template encoding an antibody or fragment thereof, and
b. isolating the mRNA by any suitable method of purification and separating reaction reagents, the DNA template, and/or mRNA product related impurities. In certain embodiments, the mRNA comprises modified nucleotides. In certain embodiments, the mRNA comprises 5′-CAP, and/or any other suitable modification.

In certain aspects, the invention provides a method for manufacturing an antibody or antigen binding fragment thereof, comprising culturing a host cell comprising a nucleic acid encoding an antibody of the invention under conditions suitable for expression of the antibody or fragment thereof and isolating said antibody or antigen binding fragment thereof.

In certain embodiments, the antibodies can be combined in one trispecific antibody with each arm of the antibody expressing a fragment of one of the antibodies in Tables 3-6, 10-11. See Ling Xu, et al. Science 6 Oct. 2017: Vol. 358, Issue 6359, pp. 85-90 DOI: 10.1126/science.aan8630.

In certain embodiments, the antibodies can be combined in various forms of bi-specific antibodies such as DARTS or other bispecific designs. See e.g. J Clin Invest 2015 Nov. 2;125 (11): 4077-90, doi: 10.1172/JCI82314. Epub 2015 Sep. 28, Bispecific Antibodies Targeting Different Epitopes on the HIV-1 Envelope Exhibit Broad and Potent Neutralization. Asokan M, Rudicell R S, Louder M, McKee K, O'Dell S, Stewart-Jones G, Wang K, Xu L, Chen X, Choe M, Chuang G, Georgiev I S, Joyce M G, Kirys T, Ko S, Pegu A, Shi W, Todd J P, Yang Z, Bailer R T, Rao S, Kwong P D, Nabel G J, Mascola J R. J Virol. 2015 December;89 (24): 12501-12, doi: 10.1128/JVI.02097-15. Epub 2015 Oct. 7.PMID: 26446600.

In certain aspect the invention provides a recombinant coronavirus monoclonal antibody, or an antigen binding fragment thereof, which binds to coronavirus spike protein and comprises a variable heavy (VH) domain and a variable light (VL) domain that have amino acid sequences that have an overall 80% sequence identity to the VH and VL domains of an antibody listed in Tables 3-6, 10 or 11, or wherein the VH domain and VL domain each have at least 80% sequence identity to the VH and VL domains, respectively, of an antibody listed in Tables 3-6, 10 or 11. In certain embodiments the antibodies are neutralizing antibodies. In certain embodiments, the antibody specifically binds to RBD of the coronavirus spike protein. In certain embodiments, the RBD binding antibodies block ACE2 receptor interaction. In certain embodiments, the antibody specifically binds to NTD of the coronavirus spike protein. In certain embodiments, the NTD antibodies do not display ADE. In certain embodiments the NTD antibodies mediate ADCC or other FcR-mediated anti-viral activity. In certain embodiments, the antibody specifically binds to the S2 of the coronavirus spike protein. In certain embodiments, the S2 antibodies mediate neutralization of certain coronaviruses. In certain embodiments the S2 antibodies mediate ADCC or other FcR-mediated anti-viral activity. In certain embodiments, the antibody is specific for CoV2. In certain embodiments the antibody is crossreactive with other coronaviruses, e.g. SARS-COV-1, MERS-COV, 229E, NL63, HKU1, OC43, bat coronavirus, and/or pangolin coronavirus. In certain embodiments, the antibody, or the antigen-binding fragment thereof, wherein the concentration of the antibody, or antigen-binding fragment thereof, required for 50% neutralization of coronavirus, e.g but not limited to CoV2 virus, (IC50) is as described in Table 4. In certain embodiments, IC50 is up to about 1 microg/ml, up to about 500 ng/ml, up to about 250 ng/ml, up to about 100 ng/ml or up to about 50 ng/ml.

In non-limiting embodiments, the antibody, or antigen binding fragment thereof binds to coronavirus domain RBD, NTD, or S2. In certain embodiments the binding specificity is as described in Table 4.

The antibody, or the antigen-binding fragment thereof, according to any of claims 1-4, wherein the antibody, or antigen-binding fragment thereof, is a recombinant human monoclonal antibody.

In certain aspects the invention provides recombinant coronavirus antibody or the antigen binding fragment thereof, as described in Table 4, e.g. without limitation DH1046, DH1047, DH1073, DH1235.

In certain embodiments the antibody or antigen binding fragment thereof, comprises a heavy chain (VH) comprising at least one CDRH1, at least one CDRH2 and at least one CDRH3 and a light chain (VL) comprising at least one CDRL1, at least one CDRL2 and at least one CDRL3, wherein at least one CDR, comprises, consists essentially of or consists of an amino acid sequence according to any of the sequences listed in FIGS. 20-25, 46, 38, 45, or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional sequence variant has 80%, 81%, 82%, 83%, 84%, 85%, 86%, 87%, 88%, 89%, 90%, 91%, 92%, 93%, 94%, 95%, 96%, 97%, 98% or 99% identity.

In certain embodiments the antibody or antigen binding fragment thereof, comprises a heavy chain (VH) comprising CDRH1, CDRH2 and CDRH3 and a light chain (VL) comprising CDRL1, CDRL2 and CDRL3, wherein the CDR, comprises, consists essentially of or consists of an amino acid sequence according to any of the sequences listed in FIGS. 20-25, 46, 38, 45, or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional sequence variant has 80%, 81%, 82%, 83%, 84%, 85%, 86%, 87%, 88%, 89%, 90%, 91%, 92%, 93%, 94%, 95%, 96%, 97%, 98% or 99% identity.

In certain embodiments, (a) VI domain CDRL1-3 regions together have no more than 5, 6, 7, 8, 9, or 10 amino acid variations as compared to the corresponding CDRL1-3 regions of an antibody listed in Table 4, (b) Vh domain CDRH1-3 regions together have no more than 5, 6, 7, 8, 9, or 10 amino acid variations as compared to the corresponding CDRH1-3 regions of the antibody listed in Table 4, and (c) the VI domain and Vh domain framework regions are derived from a human antibody.

In certain embodiments, (a) VI domain CDRL1-3 regions together have 1, 2, 3, 4 5, 6, 7, 8, 9, or 10 amino acid variations as compared to the corresponding CDRL1-3 regions of an antibody listed in Table 4, (b) Vh domain CDRH1-3 regions together have 1, 2, 3, 4 5, 6, 7, 8, 9, or 10 amino acid variations as compared to the corresponding CDRH1-3 regions of the antibody listed in Table 4, and (c) the VI domain and Vh domain framework regions are derived from a human antibody.

In certain embodiments, (a) VI domain framework regions together have 1, 2, 3, 4 5, 6, 7, 8, 9, or 10 amino acid variations as compared to the corresponding CDRL1-3 regions of an antibody listed in Table 4, (b) Vh domain framework regions together have 1, 2, 3, 4 5, 6, 7, 8, 9, or 10 amino acid variations as compared to the corresponding CDRH1-3 regions of the antibody listed in Table 4, and (c) the VI domain and Vh domain framework regions are derived from a human antibody.

In certain embodiments, the antibody or antigen binding fragment thereof, comprises a heavy chain comprising at least one CDRH1, at least one CDRH2 and at least one CDRH3 and a light chain comprising at least one CDRL1, at least one CDRL2 and at least one CDRL3, wherein at least one CDR, comprises, consists essentially of or consists of an amino acid sequence according to any of paired VHH712384 and VI K711897 (DH1047) sequences in Table 6 and FIG. 20, VhDH1073 VH and VI DH1073 VK in FIG. 46C, Vh DH1235 VH and VI DH1235 VK in FIG. 46C, Vh H026103 and VI K023879 (DH1042) sequences in Table 6 and FIG. 20, VH H026124 and VL L024055 (DH1041) sequences in Table 6 and FIG. 20, VH H026116 and VL K023888 (DH1043) in FIG. 20 or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional sequence variant has 80%, 81%, 82%, 83%, 84%, 85%, 86%, 87%, 88%, 89%, 90%, 91%, 92%, 93%, 94%, 95%, 96%, 97%, 98% or 99% identity.

In certain embodiments, the antibody or antigen binding fragment thereof, the antibody or antigen binding fragment thereof, comprises, consists essentially of or consists of paired VHH712384 and VI K711897 (DH1047) sequences in Table 6 and FIG. 20, VhDH1073 VH and VI DH1073 VK in FIG. 46C, Vh DH1235 VH and VI DH1235 VK in FIG. 46C, Vh H026103 and VI K023879 (DH1042) sequences in Table 6 and FIG. 20, VH H026124 and VL L024055 (DH1041) sequences in Table 6 and FIG. 20, VH H026116 and VL K023888 (DH1043) in FIG. 20 or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments the functional sequence variant has 80%, 81%, 82%, 83%, 84%, 85%, 86%, 87%, 88%, 89%, 90%, 91%, 92%, 93%, 94%, 95%, 96%, 97%, 98% or 99% identity.

In certain embodiments, the antibody or antigen binding fragment thereof comprises, consists essentially of or consists of paired VHH712384 and VI K711897 (DH1047) sequences in Table 6 and FIG. 20, VhDH1073 VH and VI DH1073 VK in FIG. 46C, Vh DH1235 VH and VI DH1235 VK in FIG. 46C, Vh H026103 and VI K023879 (DH1042) sequences in Table 6 and FIG. 20, VH H026124 and VL L024055 (DH1041) sequences in Table 6 and FIG. 20, VH H026116 and VL K023888 (DH1043) in FIG. 20.

In certain embodiments, the antibody or antigen binding fragment thereof is any one of the antibodies from Table 4. In certain embodiments, the antibody is DH1043, DH1042, DH1041, DH1050 or DH1050.1, DH1051, or DH1046. In certain embodiments, the antibody is DH1041 or DH1043 or DH1057. In certain embodiments, the antibody is DH1047, DH1073 or DH1235.

The antibody, or the antigen binding fragment thereof, according to any of the previous paragraphs, wherein the antibody, or the antigen binding fragment thereof, is a purified antibody, a single chain antibody, Fab, Fab′, F(ab′)2, Fv or scFv.

In certain aspects, the invention provides an in vitro transcription system to synthesize ribonucleic acids (RNAs) encoding antibodies of the invention, comprising: a reaction vessel, a DNA vector template comprising nucleic acid sequence encoding an antibody of the invention as described in any of the preceding claims, and reagents for carrying out an in vitro transcription reaction that produces mRNA encoding an antibody or fragment thereof of the invention. In certain embodiments the mRNA is modified mRNA.

In certain aspects, the invention provides a method for manufacturing an mRNA encoding an antibody or antigen binding fragment thereof, comprising:
a. providing an in vitro transcription reaction vessel comprising a DNA template encoding an antibody or fragment thereof according to any of the preceding claims and reagents under conditions suitable for in vitro transcription of the nucleic acid template, thereby producing an mRNA template encoding the antibody or fragment thereof according to any of the preceding claims, and
b. isolating the mRNA by any suitable method of purification and separating reaction reagents, the DNA template, and/or mRNA product related impurities. In certain embodiments, the mRNA comprises modified nucleotides. In certain embodiments, the mRNA comprises 5′-CAP, and/or any other suitable modification.

In certain aspects, the invention provides a method for manufacturing an antibody or antigen binding fragment thereof, comprising culturing a host cell comprising a nucleic acid according to any of the preceding claims under conditions suitable for expression of the antibody or fragment thereof and isolating said antibody or antigen binding fragment thereof.

In certain aspects, the invention provides a method of treating or protecting against coronavirus infection comprising administering a composition comprising an antibody or fragment thereof comprising a Vh or VI sequence of any of the antibodies of the invention, including without limitation Vh and VI sequences comprised in a bi- or tri-specific antibody format, or multivalent antibody forms.

In certain aspects the invention provides multivalent antibodies comprising any of antibodies or antigen binding fragments described herein. In certain aspects the invention provides multispecific antibodies or antigen binding fragments thereof comprising any of the antibodies described herein. In non-limiting embodiments, the multispecific antibody comprises fragments from DH1047. In non-limiting embodiments, the multispecific antibody comprises fragments from DH1047 and DH1073. In non-limiting embodiments, the multispecific antibody comprises fragments from DH1073.

In certain aspects the invention provides therapeutic and/or prophylactic methods comprising administering a therapeutic and/or prophylactic amount in a subject in need thereof anyone of the antibodies or antigen binding fragments of the invention, including without limitation a purified antibody IgG antibody, a multivalent antibody, multispecific antibody, a single chain antibody, Fab, Fab′, F(ab′)2, Fv or scFv, or any combination thereof.

A recombinant coronavirus monoclonal antibody, or an antigen binding fragment thereof, which binds to coronavirus spike protein and comprises a variable heavy (VH) domain and a variable light (VL) domain that have amino acid sequences that have an overall 80% sequence identity to the VH and VL domains of an antibody listed in Tables 3-6, 10 or 11, or wherein the VH domain and VL domain each have at least 80% sequence identity to the VH and VL domains, respectively, of an antibody listed in Tables 3-6, 10 or 11.

In certain aspects, the invention provides a recombinant coronavirus monoclonal antibody, or an antigen binding fragment thereof, which binds coronavirus spike protein, which comprises:
a. Vh domain CDRH1-3 regions from an antibody listed in Table 4; and/or
b. VI domain CDRL1-3 regions from an antibody listed in Table 4, wherein the Vh and VI are from the same antibody; and
c. Wherein the framework of the variable heavy (Vh) domain comprises amino acid sequences that have at least 90% sequence identity to the V gene, D gene and J gene of the Vh gene of the corresponding antibody from which the CDRs are derived and wherein the framework of the variable light (VI) domain comprises amino acid sequences that have at least 90% sequence identity to the V and J genes of the VI gene from the corresponding antibody from which the CDRs are derived. In non-limiting embodiments, the antibody is DH1047, DH1042, DH1046, or DH1073.

In certain aspects, the invention provides a recombinant coronavirus monoclonal antibody, or an antigen binding fragment thereof, which binds coronavirus spike protein, which comprises:
a. Vh domain CDRH1-3 regions from an antibody listed in Table 4; and/or
b. VI domain CDRL1-3 regions from an antibody listed in Table 4, wherein the Vh and VI are from the same antibody; and
c. Wherein the framework of the variable heavy (Vh) domain comprises amino acid sequences that have at least 80%, 85%, 90%, 91%, 92%, 93%, 94%, 95%, 96%, 97%, 98%, or 99% sequence identity to the V gene, D gene and J gene making up the Vh gene of the corresponding antibody from which the CDRs are derived and wherein the framework portions of the variable light (VI) domain comprises amino acid sequences that have at least 80%, 85%, 90%, 91%, 92%, 93%, 94%, 95%, 96%, 97%, 98%, or 99% sequence identity to the V and J genes making up the VI gene from the corresponding antibody from which the CDRs are derived. In non-limiting embodiments, the antibody is DH1047, DH1042, DH1046, or DH1073.

In certain aspects, the invention provides a recombinant coronavirus monoclonal antibody, or an antigen binding fragment thereof, which binds coronavirus spike protein, which comprises:
a. Vh domain CDRH1-3 regions from an antibody listed in Table 4; and/or
b. VI domain CDRL1-3 regions from an antibody listed in Table 4, wherein the Vh and VI are from the same antibody; and
c. Wherein the framework of the variable heavy (Vh) domain comprises amino acid sequences that have 80%, 85%, 90%, 91%, 92%, 93%, 94%, 95%, 96%, 97%, 98%, or 99% sequence identity to the V gene, D gene and J gene making up the Vh gene of the corresponding antibody from which the CDRs are derived and wherein the framework portions of the variable light (VI) domain comprises amino acid sequences that have 80%, 85%, 90%, 91%, 92%, 93%, 94%, 95%, 96%, 97%, 98%, or 99% sequence identity to the V and J genes making up the VI gene from the corresponding antibody from which the CDRs are derived. In non-limiting embodiments, the antibody is DH1047, DH1042, DH1046, or DH1073.

In certain embodiments, the antibody is DH1047 and the framework of the variable heavy (Vh) domain comprises, consists essentially of, consists of or has amino acid sequences derived from human IGHV1-46 and IGHJ4 Ig genes (See e.g. Table 3, Table 4D, FIG. 45) and wherein the framework of the variable light (VI) domain comprises amino acid sequences derived from IGKV4-1 and IGKJI human IgG genes (See e.g. Table 3, Table 4D, FIG. 45).

In certain embodiments, the antibody is DH1073 and the framework of the variable heavy (Vh) domain comprises, consists essentially of, consists of or has amino acid sequences derived from human IGHV1-46 and IGHJ6 Ig genes (See e.g. Table 4D, FIG. 45) and wherein the framework of the variable light (VI) domain comprises, consists essentially of, consists of or has amino acid sequences derived from IGKV3-11 and IGKJ1 human IgG genes (Table 4D, FIG. 45)

In certain embodiments, the antibody is DH1042 and the framework of the variable heavy (Vh) domain comprises, consists essentially of, consists of or has amino acid sequences derived from IGH1-69 and IGHJ6 human Ig genes (See e.g. Table 4D, FIG. 45) and wherein the framework of the variable light (VI) domain comprises, consists essentially of, consists of or has amino acid sequences derived from IGKV1-39 and IGKJ2 human IgG genes (See e.g. Table 4D, FIG. 45).

In certain embodiments, the recombinant antibody or the antigen binding fragment thereof is DH1046, DH1041, DH14043, or DH1235, and wherein the framework of the variable heavy (Vh) domain comprises, consists essentially of, consists of or has amino acid sequences derived from the corresponding human Ig genes described in FIG. 38, 45 or Table 3, Table 4 and wherein the framework of the variable light (VI) domain comprises, consists essentially of, consists of or has amino acid sequences derived from the corresponding human Ig genes described in at least in FIG. 38, 45 or Table 3, Table 4.

In certain embodiments, the antibody or antigen binding fragment thereof, comprises a heavy chain comprising at least one CDRH1, at least one CDRH2 and at least one CDRH3 and a light chain comprising at least one CDRL1, at least one CDRL2 and at least one CDRL3, wherein at least one CDR, comprises, consists essentially of or consists of an amino acid sequence according to any of paired VHH712384 and VI K711897 (DH1047) sequences in Table 6 and FIG. 20, VhDH1073 VH and VI DH1073 VK in FIG. 46C, Vh DH1235 VH and VI DH1235 VK in FIG. 46C, Vh H026103 and VI K023879 (DH1042) sequences in Table 6 and FIG. 20, VH H026124 and VL L024055 (DH1041) sequences in Table 6 and FIG. 20, VH H026116 and VL K023888 (DH1043) in FIG. 20, or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional sequence variant has 80%, 81%, 82%, 83%, 84%, 85%, 86%, 87%, 88%, 89%, 90%, 91%, 92%, 93%, 94%, 95%, 96%, 97%, 98% or 99% identity.

In certain embodiments, the antibody or antigen binding fragment thereof, comprises, consists essentially of or consists of paired VHH712384 and VI K711897 (DH1047) sequences in Table 6 and FIG. 20, VhDH1073 VH and VI DH1073 VK in FIG. 46C, Vh DH1235 VH and VI DH1235 VK in FIG. 46C, Vh H026103 and VI K023879 (DH1042) sequences in Table 6 and FIG. 20, VH H026124 and VL L024055 (DH1041) sequences in Table 6 and FIG. 20, VH H026116 and VL K023888 (DH1043) in FIG. 20 or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments the functional sequence variant has 80%, 81%, 82%, 83%, 84%, 85%, 86%, 87%, 88%, 89%, 90%, 91%, 92%, 93%, 94%, 95%, 96%, 97%, 98% or 99% identity.

In certain embodiments, the antibody, or antigen binding fragment thereof binds to coronavirus domain RBD, NTD, or S2.

In certain embodiments, the antibody, or antigen binding fragment thereof binds RBD.

In certain embodiments, wherein the antibody, or antigen-binding fragment thereof, comprises an Fc moiety. In certain embodiments, wherein the antibody, or antigen-binding fragment thereof, comprises a mutation(s) in the Fc moiety, in certain embodiments the mutation reducing binding of the antibody to an Fc receptor, in certain embodiments the mutation increasing the half-life of the recombinant antibody. In certain embodiments, the Fc mutation is “LS” mutation. In certain embodiments, the Fc mutation is “4A” mutation.

In certain embodiments, the antibody, or the antigen binding fragment thereof, is a purified antibody IgG antibody, a multivalent antibody, multispecific antibody, a single chain antibody, Fab, Fab′, F(ab′)2, Fv or scFv. In certain embodiments, the antibody is of any isotype.

In certain aspects, the invention provides an antibody, or the antigen-binding fragment thereof for use as a medicament. In certain embodiments, the use is in the prevention and/or treatment of coronavirus infection. In certain embodiments the coronavirus is CoV2.

In certain aspects, the invention provides a nucleic acid molecule comprising a polynucleotide encoding the antibody, or the antigen-binding fragment thereof, according to any of the preceding paragraphs.

In certain embodiments, the polynucleotide sequence comprises, consists essentially of or consists of a nucleic acid sequence according to any one of the sequences in FIGS. 20-26, 46, 38 and 45; or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional variation is 80%, 81%, 82%, 83%, 84%, 85%, 86%, 87%, 88%, 89%, 90%, 91%, 92%, 93%, 94%, 95%, 96%, 97%, 98% or 99% identity.

In certain embodiments the nucleic acid is a ribonucleic acids (RNA). In certain embodiments the RNA is mRNA which suitable for use and delivery as a therapeutic mRNA. In certain embodiment, the mRNA comprises a 5′-terminal CAP modification. In certain embodiments the mRNA comprises modified nucleotides. In certain embodiments, the mRNA is formulated in a lipid nanoparticle.

In certain aspects, the invention provides a vector comprising the nucleic acid molecule according to any of the preceding paragraphs.

In certain aspects, the invention provides a cell expressing the antibody, or the antigen binding fragment thereof, according to any of the preceding claims; or comprising a vector of the invention.

In certain aspects, the invention provides a pharmaceutical composition comprising the antibody, or the antigen binding fragment thereof, a nucleic acid encoding the same, a vector comprising the nucleic acid and/or a cell comprising the nucleic acid or vector, and optionally a pharmaceutically acceptable carrier. In certain embodiments the antibody is DH1043, DH1042, DH1041, DH1046, DH1047, DH1073, DH1235 or a combination thereof.

In certain aspects, the invention provides a pharmaceutical composition comprising the antibody, or the antigen binding fragment thereof, wherein the antibody, or the antigen binding fragment thereof, comprise a Vh and VI sequence of any of the preceding claims, wherein the Vh and VI sequences form a multivalent or multispecific antibody.

In certain aspects, the invention provides a pharmaceutical composition comprising at least one RBD binding antibody of the invention and at least one NTD binding antibody of the invention. In non-limiting embodiments, the RBD antibody is any one of the antibodies in Table 4, a fragment of variant thereof. In non-limiting embodiments, the NTD antibody is any of the antibodies in Table 4, a fragment or variant thereof.

In certain aspects, the invention provides a pharmaceutical composition comprising two RBD binding antibodies or antigen binding fragments thereof, wherein the two antibodies or antigen binding fragments thereof have non-overlapping epitopes. In certain embodiments one of the antibodies is DH1073. In certain embodiments the second RBD antibody is DH1046, DH1047, DH1042 or any other RBD binding antibody.

In certain embodiments the compositions further comprises a pharmaceutically acceptable excipient, diluent, adjuvant or carrier.

In certain aspects, the invention provides a method of treating or preventing coronavirus infection in a subject in need thereof, comprising administering the antibody, or the antigen binding fragment thereof, a nucleic acid encoding the same, a vector comprising the nucleic acid and/or a cell comprising the nucleic acid or vector, or a pharmaceutical composition of the invention in an amount suitable to effect treatment or prevention of coronavirus infection.

In certain embodiments, the antibody is administered prior to coronavirus exposure or at the same time as coronavirus exposure.

In certain aspects, the invention provides a method of treating or protecting against coronavirus infection comprising administering therapeutic or prophylactic amount of a composition comprising an antibody or antigen binding fragment thereof comprising a Vh and VI sequence of any of the preceding paragraphs, wherein the Vh and VI sequences are comprised in a bi- or tri-specific antibody format and/or in a multivalent format. In certain aspects the invention provides, nucleic acid sequences encoding these bi- or tri-specific antibody formats and/or in a multivalent formats, including modified mRNAs suitable for pharmaceutical use and delivery.

Use of the multispecific or multivalent antibodies or fragments thereof, administer as recombinant proteins, nucleic acids, including without limitation mRNAs, or a combination thereof in prophylactic or therapeutic methods. The nucleic acids and recombinant proteins can be formulated in any suitable formulation, and optionally comprise an adjuvant. In some embodiments, the adjuvant is LNP. In some embodiments, the formulation comprises LNPs.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1K show representative data of FcR-dependent and FcR-independent SARS-CoV-2 infection-enhancement mediated by RBD and NTD antibodies. (FIGS. 1A-B) Timeline of blood sampling and antibody isolation from convalescent SARS-COV-2 and SARS-COV-1 donors. Plasmablasts and/or antigen-specific memory B cells (MBC) were sorted from a (FIG. 1A) SARS-CoV-2 infected individual (SARS-COV-2 donor) and a (FIG. 1B) 2003 SARS survivor (SARS-CoV-1 donor). (FIG. 1C) Summary of number and specificity of antibodies isolated from each donor. (FIGS. 1D-E) FcR-independent SARS-COV-2 infection-enhancement mediated by non-neutralizing NTD antibodies. In vitro neutralization curves for NTD infection-enhancing antibodies against (FIG. 1D) pseudotyped SARS-COV-2 D614G in 293T-hACE2 cells, and (FIG. 1E) replication-competent nano-luciferase (nLuc) SARS-COV-2 in Vero cells. (FIGS. 1F-K) FcR-dependent SARS-COV-2 infection-enhancement in ACE2-negative cells mediated by neutralizing RBD antibodies. Pseudotyped SARS-COV-2 incubated with RBD antibodies or mock medium control were inoculated on (FIG. 1F) parental TZM-bl cells, and TZM-bl cells stably expressing human FcγR receptors (FIG. 1G) FcγRI, (FIG. 1H) FcγRIIa, (FIG. 11) FcγRIIb or (FIG. 1J) FcγRIII. The effect of RBD antibody fragment antigen-binding regions (Fabs) on pseudotyped SARS-COV-2 D614G infection were tested in (FIG. 1K) FcγRI-expressing TZM-bl cells and (FIG. 1L) FcγRIIb-expressing TZM-bl cells. Relative luminescence units (RLUs) were measured in cell lysate at 68-72 hours post-infection. Upward deflection of RLUs in the presence of antibody indicates FcR-mediated infection. Three or four independent experiments were performed and representative data are shown.

FIGS. 2A-2E show representative structural and phenotypic characterization of infection-enhancing and non-infection-enhancing RBD and NTD antibodies. (FIG. 2A) Summary of down-selected antibodies. Binding domain, effect (neutralizing or infection-enhancing) in ACE2-positive/FcγR-negative cells and ACE2-negative/positive cells, cross-reactivity with SARS-COV-1, ACE2 blocking activity, neutralization titers against pseudovirus and replication-competent viruses were shown. MN titer, micro-neutralization titer. (FIGS. 2B-E) 3D reconstruction of negative stain electron microscopy of SARS-COV-2 Spike trimer stabilized with 2 proline mutations (S-2P) binding to (FIG. 2B) infection-enhancing RBD antibody fabs, (FIG. 2C) non-infection-enhancing RBD antibody Fabs, (FIG. 2D) infection-enhancing NTD antibody Fabs, (FIG. 2E) non-infection-enhancing NTD antibody Fabs.

FIGS. 3A-3H show representative biophysical and structural determination that infection-enhancing and non-infection enhancing antibodies can simultaneously bind to the same S protein. (FIG. 3A) Cross-blocking activity of RBD and NTD neutralizing antibodies tested by surface plasmon resonance (SPR). SARS-COV-2 S-2P trimer was captured by the antibody on the Y-axis followed by binding by the antibody on the X-axis. We defined competition if the binding antibody did not bind the captured S protein. (FIG. 3B) 3D reconstruction of simultaneous recognition of SARS-COV-2 S-2P trimer by two RBD antibodies DH1041+DH1047, or DH1043+DH1047. All three antibodies are infection-enhancing in ACE2-negative/FcγR-positive cells but neutralizing SARS-COV-2 in ACE2-positive/FcγR-negative cells. (FIG. 3C) Cross-blocking activity of neutralizing antibodies and infection-enhancing NTD antibodies tested by SPR. SARS-COV-2 S-SP trimer was captured by the antibody on the Y-axis followed by binding by the antibody on the X-axis. We defined competition if the binding antibody did not bind the captured S protein. (FIG. 3D) 3D reconstruction of simultaneous recognition of SARS-COV-2 S protein by NTD antibodies DH1053+DH1050.1 combination. In ACE2-positive/FcγR-negative cells, DH1050.1 is neutralizing while DH1053 enhances SARS-COV-2 infection. Both antibodies have no effect in ACE2-negative/FcγR-positive cells. (FIG. 3E) 3D reconstruction of simultaneous recognition of SARS-COV-2 S protein by RBD infection-enhancing antibody+NTD non-infection-enhancing antibody combinations. All of them are neutralizing antibodies in ACE2-positive/FcγR-negative cells. (FIG. 3F) 3D reconstruction of simultaneous recognition of SARS-CoV-2 S protein by three antibody combinations RBD antibody DH1043+RBD antibody DH1047+NTD antibody DH1051 or DH1050.1. Both RBD antibodies are infection-enhancing in ACE2-negative/FcγR-positive cells but neutralizing SARS-COV-2 in ACE2-positive/FcγR-negative cells. DH1051 and DH1050.1 are neutralizing NTD antibodies in ACE2-negative/FcγR-positive cells but have no effect in ACE2-negative/FcγR-positive cells. (FIGS. 3G-H) Effect of combining infection-enhancing RBD and NTD antibodies on SARS-COV-2 pseudovirus infection in ACE2-expressing cells. The infection-enhancing NTD antibody DH1052 was mixed with infection-enhancing RBD antibodies DH1041 (FIG. 3G) or DH1043 (FIG. 3H) in 1:132 ratio or 1:1,325 ratio, respectively. The NTD: RBD antibody mixtures (orange), as well as RBD antibody alone (blue), were five-fold serially diluted and tested for neutralization against SARS-COV-2 D614G pseudoviruse in 293T/ACE2 cells.

FIGS. 4A-4E show cryo-electron microscopy of neutralizing and non-neutralizing antibodies in complex with SARS-COV-2 Spike ectodomain. Structures of SARS-COV-2 S protein in complex with RBD antibodies (FIG. 4A) DH1041 (red), (FIG. 4B) DH1043 (pink), (FIG. 4C) DH1047 (magenta), (FIG. 4D) neutralizing NTD antibody DH1050.1 (blue), and (FIG. 4E) infection-enhancing NTD antibody DH1052 (green). Each antibody is bound to Spike ectodomain stabilized with 2 proline mutations (S-2P) shown in gray with its Receptor Binding Motif (RBM) colored purple blue. (Right) Zoomed-in views of the antibody interactions with S-2P trimers. The antibody complementarity determining (CDR) loops are colored: HCDRI yellow, HCDR2 limon, HCDR3 cyan, LCDR1 orange, LCDR2 wheat and LCDR3 light blue.

FIGS. 5A-5M show representative data. NTD antibody DH1052 enhances SARS-COV-2 infection in vitro, but does not enhance SARS-COV-2 replication or disease in vivo. (FIGS. 5A-F) Intentionally left blank (FIGS. 5G-L) Reduction of SARS-COV-2 replication and disease by prophylactic administration of an NTD NAb DH1050.1 or an in vitro infection-enhancing NTD Ab DH1052. (FIG. 5G) Study design. Cynomolgus macaques (n=5 per group) were infused with DH1052, DH1050.1 or an irrelevant control CH65 antibody 3 days before 105 PFU of SARS-CoV-2 challenge via intranasal route and intratracheal route. Viral load including viral RNA and subgenomic RNA (sgRNA) were measured on the indicated pre-challenge and post-challenge timepoints. Lungs were harvested on Day 4 post-challenge for histopathology study. (FIG. 5H) Lung histopathology. Sections of the left caudal (Lc), right middle (Rm), and right caudal (Rc) lung were evaluated and scored for the presence of inflammation by hematoxylin and eosin (H&E) staining, and for the presence of SARS-COV-2 antigen by immunohistochemistry (IHC) staining. Dots indicate the sums of Lc, Rm, and Rc scores in each animal. (FIGS. 5I-J) SARS-COV-2 (FIG. 1) envelope gene (E gene) sgRNA and (FIG. 5J) nucleocapsid gene (N gene) sgRNA in bronchoaveolar lavage (BAL) on Day 2 and Day 4 post challenge. (FIGS. 5K-L) SARS-COV-2 (FIG. 5K) E gene sgRNA and (FIG. 5L) N gene sgRNA in nasal swab on Day 2 and Day 4 post challenge. Statistical significance in all the panels were determined using Wilcoxon rank sum exact test. Asterisks show the statistical significance between indicated group and CH65 control group: ns, no significance, *P<0.05, ** P<0.001, *** P<0.0001.

FIGS. 6A-6K show representative graphs of data. The Fc-dependent infection-enhancing RBD antibodies not enhance but protect mice and non-human primates from SARS-COV-2 challenge. (FIGS. 6A-D) Intentionally left blank (FIGS. 6E-J) RBD NAbs and infection-enhancing Abs protected SARS-COV-2 infection in non-human primates. (FIG. 6E) Study design. Cynomolgus macaques (n=5 per group) were infused with DH1041, DH1043, DH1046, DH1047 or an irrelevant CH65 antibody 3 days before 105 PFU of SARS-COV-2 challenge via intranasal route and intratracheal route. Viral load including viral RNA and subgenomic RNA (sgRNA) were measured on the indicated pre-challenge and post-challenge timepoints. Lungs were harvested on Day 4 post-challenge for histopathology study. (FIGS. 6F-G) Lung histopathology. Sections of the left caudal (Lc), right middle (Rm), and right caudal (Rc) lung were evaluated and scored for (FIG. 6F) the presence of inflammation by hematoxylin and eosin (H&E) staining, and (FIG. 6G) for the presence of SARS-COV-2 antigen by immunohistochemistry (IHC) staining. Dots indicate the sums of Lc, Rm, and Rc scores in each animal. (FIGS. 6H-I) SARS-COV-2 (FIG. 6H) envelope gene (E gene) sgRNA and (I) nucleocapsid gene (N gene) sgRNA in bronchoaveolar lavage (BAL) on Day 2 and Day 4 post challenge. (FIGS. 6J-K) SARS-COV-2 (FIG. 6J) E gene sgRNA and (FIG. 6K) N gene sgRNA in nasal swab on Day 2 and Day 4 post challenge. Statistical significance in all the panels were determined using Wilcoxon rank sum exact test. Asterisks show the statistical significance between indicated group and CH65 control group: ns, no significance, *P<0.05, ** P<0.001.

FIGS. 7A-7D show graphs of representative data. The Fc-dependent infection-enhancing RBD antibodies not enhance but protect mice and non-human primates from SARS-COV-2 challenge. (FIGS. 7A-D) Intentionally left blank (FIGS. 7E-J) RBD NAbs and infection-enhancing Abs protected SARS-COV-2 infection in non-human primates. (FIG. 7E) Study design. Cynomolgus macaques (n=5 per group) were infused with DH1041, DH1043, DH1046, DH1047 or an irrelevant CH65 antibody 3 days before 105 PFU of SARS-COV-2 challenge via intranasal route and intratracheal route. Viral load including viral RNA and subgenomic RNA (sgRNA) were measured on the indicated pre-challenge and post-challenge timepoints. Lungs were harvested on Day 4 post-challenge for histopathology study. (FIGS. 7F-G) Lung histopathology. Sections of the left caudal (Lc), right middle (Rm), and right caudal (Rc) lung were evaluated and scored for (FIG. 7F) the presence of inflammation by hematoxylin and eosin (H&E) staining, and (FIG. 7G) for the presence of SARS-COV-2 antigen by immunohistochemistry (IHC) staining. Dots indicate the sums of Lc, Rm, and Rc scores in each animal. (FIGS. 7H-I) SARS-COV-2 (FIG. 7H) envelope gene (E gene) sgRNA and (FIG. 71) nucleocapsid gene (N gene) sgRNA in bronchoaveolar lavage (BAL) on Day 2 and Day 4 post challenge. (J-K) SARS-COV-2 (FIG. 7J) E gene sgRNA and (FIG. 7K) N gene sgRNA in nasal swab on Day 2 and Day 4 post challenge. Statistical significance in all the panels were determined using Wilcoxon rank sum exact test. Asterisks show the statistical significance between indicated group and CH65 control group: ns, no significance, *P<0.05, ** P<0.001.

FIGS. 8A-8D show flow cytometry plots. Isolation of SARS-COV-2-reactive antibodies from single cell-sorted plasmablasts and memory B cells. (FIG. 8A) Flow cytometry gating strategy for unbiased plasmablasts sorting. At day 11 and day 15 post onset of COVID-19 symptom, plasmablasts (CD14/CD16/CD3/CD235a/CD19⁺/CD20^{low}/IgD/CD27^{high}/CD38^{high}) from a SARS-COV-2 donor. (FIGS. 8B-D) Flow cytometry gating strategy for antigen specifc-memory B cells sorting. Antigen specific B cells from SARS-COV-1 and SARS-COV-2 donors were sorted with different combinations of the SARS-COV-2 S-2P, RBD, NTD probes. Representative data for sorting Spike double positive (FIG.B), Spike⁺ or NTD⁺ (Panel C), as well as RBD⁺ or NTD⁺ (Panel D) subsets were shown.

FIGS. 9A-91 show graphs of representative data. Neutralization activities of the RBD and NTD antibodies. (FIGS. 9A-D) Neutralization activity of RBD antibodies. (FIG. 9A) Proportion of SARS-COV-2 RBD antibodies (n=81) that exhibited detectable neutralization in the microneutralization assay. (FIG. 9B) Neutralization IC50 and IC80 of RBD neutralizing antibodies (NAbs) against pseudotyped SARS-COV-2. (FIG. 9C) Microneutralization titer, plaque reduction neutralization test (PRNT) IC50 and IC80 of RBD NAbs against replication-competent SARS-COV-2. Microneutralization titer was defined as the lowest antibody concentration that neutralize all the virus, or 99% inhibitory concentration (IC99). Antibodies with undetectable microneutralization titers are shown as gray symbols and nAbs are represented by blue symbols. (FIG. 9D) RBD NAbs blocking of ACE2 binding to SARS-COV-2 Spike(S) protein. Blocking titer is shown as IC50. (FIGS. 9E-F) Correlation analysis of RBD antibodies between neutralization and ACE2 blocking activities. Spearman correlation analysis were performed for (FIG. 9E) ACE2 blocking IC50 vs. PV neutralization IC50, as well as (F) for ACE2 blocking IC50 vs. SARS-COV-2 neutralization titers (indicated by the lowest concentration that shows no CPE). Purified RBD antibodies in Table S1 and S2 that have pseudovirus neutralization data (n=59) or SARS-COV-2 micro-neutralization assay data (n=80) were used in this analysis. P-value and r were indicated for each figures. (FIGS. 9G-I) Neutralization activity of NTD antibodies. (FIG. 9G) Proportion of SARS-COV-2 NTD antibodies (n=41) that exhibited detectable neutralization in the microneutralization assay. (FIG. 9H) Neutralization IC50 and IC80 of NTD neutralizing antibodies against pseudotyped SARS-COV-2. (FIG. 91) Microneutralization titer, PRNT IC50 and IC80 of NTD neutralizing antibodies against replication-competent SARS-COV-2. Antibodies with undetectable microneutralization titers are shown as gray symbols and neutralizing antibodies are represented by orange symbols. Horizontal bars represent the geometric means for each group of antibodies.

FIGS. 10A-10D show ELISA binding curves of down-selected antibodies. Different SARS-COV-2 or other CoV viral antigens were coated on plates and detected with serial diluted

(FIG. 10A) RBD infection-enhancing antibodies, (FIG. 10B) RBD non-infection-enhancing antibodies, (FIG. 10C) NTD infection-enhancing antibodies, and (FIG. 10D) NTD non-infection-enhancing antibodies.

FIGS. 11A-11H show neutralization of SARS-COV-2 and SARS-COV-1 antibodies. (FIGS. 10A-B) Neutralization curves for RBD antibodies against pseudotyped (Panel A) and replication-competent (FIG. 10B) SARS-COV-2. (FIGS. 10C-D) Neutralization curves for NTD antibodies against pseudotyped (FIG. 10C) and replication-competent (FIG. 10D) SARS-COV-2. (FIGS. 10E-H) Neutralization curves for cross-neutralizing antibodies against pseudotyped (FIG. 10E) and replication-competent (FIG. 10F) SARS-COV-2, SARS-COV-1 nanoluciferase (nLuc) virus (FIG. 10G), and Bat WIV1-CoV nLuc virus (FIG. 10H).

FIGS. 12A-121 show graphs of kinetics of RBD and NTD antibody Fabs on Spike-2P were measured by SPR (Panels A-I).

FIGS. 13A-I show graphs of kinetics of RBD and NTD antibody Fabs on HexaPro were measured by SPR (Panels A-I).

FIG. 14 shows a table of affinity of RBD and NTD antibody Fabs.

FIG. 15 shows autoreactivity tests of SARS-COV-2 RBD and NTD antibodies by AtheNA assay. A panel of different antigens (SS/A, SS/B, Sm, RNP, Jo-1, Scl-70, dsDNA, Centromere B and Histone) were tested.

FIGS. 16A-16C show comparison of RBD epitopes from NSEM. (FIG. 16A) A spike model (PDB 6ZGE) and corresponding Fab homology models were manually docked and rigidly fit into each negative stain density map. (FIG. 16B) The RBD of each model is enlarged and shown as a white surface, with the putative epitope of each antibody colored. Black outline indicates the ACE2 binding footprint. (FIG. 16C) Comparison to ACE2 footprint and epitopes of three published antibodies with similar epitopes. See main text for references.

FIGS. 17A-17C show comparison of NTD epitopes from NSEM. (FIG. 17A) A spike model (PDB 6ZGE) and corresponding Fab homology models were manually docked and rigidly fit into each negative stain density map. (FIG. 17B) The NTD of each model is enlarged and shown as a white surface, with the epitope of each antibody colored. Orange outline indicates the epitope of antibody 4A8, shown at bottom right. Outlines illustrate that the neutralizing antibodies DH1048-51 share the same epitope, whereas the infection-enhancing antibodies DH1053-56 bind a distinct epitope. (FIG. 17C) The model of spike complex with Fab 4A8 (orange ribbons, PDG 7C2L) is rigidly fit into each of the NSEM maps (transparent surfaces). The close fit of 4A8 into DH1049, DH1050.1 and DH1050.2 indicate theses have the same approach angle as 4A8, whereas DH1048 and DH1051 have slightly different approaches.

FIGS. 18A-18J shows ross-blocking assay of RBD and NTD neutralizing antibodies. (FIG. 18A) Scheme of cross-blocking decection assay by SPR. Capture antibody was immoblized on sensor chip, followed by injection of SARS-COV-2 Spike and then immediately injection of detection antibodies. Human antibody CH65 was used as negative control antibody in all SPR experiments. (FIGS. 18B-D) RBD antibodies DH1041 (FIG. 18B), DH1043 (FIG. 18C), or DH1044 (FIG. 18D) was immoblized as capture antibodies to test cross-blocking with different detection antibodies. Three independent experiments were shown. (FIGS. 18E,F) RBD cross-reactive antibodies DH1046 (FIG. 18E) or DH1046 (FIG. 18F) was immoblized as capture antibodies to test cross-blocking with different detection antibodies as indicated. (G-J) NTD antibodies DH1048 (FIG. 18G), DH1050.1 (FIG. 18H), DH1051 (FIG. 181), or NTD ADE antibody DH1052 (FIG. 18J) was immoblized as capture antibodies to test cross-blocking with different detection antibodies. Two or three independent experiments were shown.

FIGS. 19A-19F show ross-blocking assay of RBD and NTD neutralizing antibodies (Related to FIG. 3C). (FIG. 19A) Scheme of cross-blocking decection assay by SPR. Capture antibody was immoblized on sensor chip, followed by injection of SARS-COV-2 Spike and then immediately injection of detection antibodies. Human antibody CH65 was used as negative control antibody in all SPR experiments. (FIGS. 19B-G) SPR cross-blocking assays. NTD NAb DH1050.1 (FIG. 19B), NTD infection-enhancing abs DH1052 (FIG. 19C), DH1053 (FIG. 19D), DH1054 (FIG. 19E), DH1055 (FIG. 19F) or DH1056 (FIG. 19G) was immoblized as capture antibodies to test cross-blocking with different detection antibodies.

FIG. 20 shows heavy and light chain amino acid sequences of antibodies in Tables 3, 4A and 5. The VH and VL sequences are highlighted in grey. CDRs can be identified by any method known in the art. In some embodiment, CDRs as identified are identified using IMGT. Sequence ID numbers are shown in the figure.

FIG. 21 shows one embodiment of heavy and light chain nucleic acid sequences of antibodies in Tables 3, 4A and 5. The VH and VL sequences are highlighted in grey. Sequence ID numbers are shown in the figure.

FIG. 22 shows heavy and light chain amino acid sequences of antibodies in Table 4B. The VH and VL sequences are highlighted in grey. CDRs can be identified by any method known in the art. In some embodiment, CDRs as identified are identified using IMGT. Sequence ID numbers are shown in the figure.

FIG. 23 shows one embodiment of nucleic acid sequences of FIG. 22. The VH and VL sequences are highlighted in grey. Sequence ID numbers are shown in the figure.

FIGS. 24A-F show COVID Antibody Sequences in in vitro transcription (IVT) plasmids. Sequences of DH1041, DH1043, and DH1042 in In Vitro Transcription (IVT) Plasmids. DNA/mRNA/AA sequences for modified RNA delivery.

FIG. 25 shows maps and sequences of In Vitro Transcription (IVT) Plasmids.

FIG. 26 shows graphs of representative data. Effect of combining infection-enhancing RBD and NTD antibodies on SARS-COV-2 pseudovirus infection in ACE2-expressing cells (related to FIG. 3G-H). The infection-enhancing NTD antibody DH1052 was tested alone (FIG. 26A) or mixed with infection-enhancing RBD antibodies DH1041 (FIG. 26B) or DH1043 (FIG. 26C) in 1:12.5 ratio or 1:13250 ratio, respectively. The NTD: RBD antibody mixtures (orange), as well as RBD antibody alone (blue), were five-fold serially diluted and tested for neutralization against SARS-COV-2 D614G pseudoviruse in 293T/ACE2 cells.

FIG. 27 shows cryo-EM data collection and refinement statistics.

FIG. 28 shows cryo-EM data processing details. (left) Representative micrograph, (middle) CTF fit and (right) Representative 2D class averages for (FIG. 28A) DH1041-Spike-2P (S2P) complex, (FIG. 28B) DH1043-S2P complex, (FIG. 28C) DH1047-S2P complex, (FIG. 28D) DH1050.1-S2P complex, (FIG. 28E) DH1052-S2P complex.

FIG. 29 shows global and Local map resolutions for DH1041/S-2P complex. (FIG. 29A) Cryo-EM reconstruction of DH1041 bound to 1-RBD-up 2P spike. (FIG. 29B) Cryo-EM reconstruction of DH1041 bound to 2-RBD-up 2P spike. (FIG. 29C) Cryo-EM reconstruction of DH1041 bound to 3-RBD-up 2P spike.

FIG. 30 shows global and Local map resolutions for DH1043/S-2P complex. (FIG. 30A) Cryo-EM reconstructions of DH1043 bound to 1-RBD-up 2P spike Top two rows show refined maps, bottom row shows the FSC curve for each corresponding map. (FIG. 30B) Refined cryo-EM map that was used for model building colored by local resolution. (FIG. 30C) Zoomed-in view of the DH1043 interface with RBD. The cryo-EM map is shown as a blue mesh with underlying fitted model shown in cartoon representation, with the DH1047 HCDRI loop colored yellow, HCDR2 colored limon and HCDR3 cyan.

FIG. 31 shows global and Local map resolutions for DH1047/S-2P complex. (FIG. 31A) Cryo-EM reconstructions of DH1047 bound to 3-RBD-up 2P spike. Top two rows show refined maps, bottom row shows the FSC curve for each corresponding map. (FIG. 31B) Refined cryo-EM map that was used for model building colored by local resolution. (FIG. 31C) Zoomed-in view of the DH1047 interface with RBD. The cryo-EM map is shown as a blue mesh with underlying fitted model shown in cartoon representation, with the DH1047 HCDRI loop colored yellow, HCDR2 colored limon, HCDR3 cyan and LCDR1 orange.

FIG. 32 shows global and Local map resolutions for DH1050.1/S-2P complex. (FIG. 32A) Cryo-EM reconstruction of DH1050.1 bound to 3-RBD-down 2P spike. (FIG. 32B) Fourier shell correlation curves. (FIG. 32C) Refined cryo-EM map colored by local resolution for the DH1050.1 bound to 3-RBD-down 2P spike. (FIG. 32D) Cryo-EM reconstruction of DH1050.1 bound to 1-RBD-up 2P spike. (FIG. 32E) Fourier shell correlation curves. (F) Refined cryo-EM map colored by local resolution for the DH1050.1 bound to 1-RBD-up 2P spike.

FIG. 33 shows lobal and Local map resolutions for DH1052/S-2P complex. (FIG. 33A) Cryo-EM reconstruction of DH1052 bound to 3-RBD-down stabilized Spike “2P” (S-2P). (FIG. 33B) Fourier shell correlation curves. (FIG. 33C) Refined cryo-EM map colored by local resolution for the DH1052 bound to 3-RBD-down S-2P. (FIG. 33D) Cryo-EM reconstruction of DH1052 bound to 1-RBD-up S-2P. (FIG. 33E) Fourier shell correlation curves. (FIG. 33F) Refined cryo-EM map colored by local resolution for the DH1052 bound to 1-RBD-up S-2P.

FIG. 34 shows pathology scoring of the non-human primates that treated with RBD or NTD NAbs and infected with SARS-COV-2 (Related to FIGS. 5H-I and 6F-G). Lung histopathology. Sections of the left caudal (Lc), right middle (Rm), and right caudal (Rc) lung were evaluated and scored in a 1-4 scoring system for the presence of inflammation by Hematoxylin and eosin (H&E) staining, and for the presence of SARS-COV-2 antigen by Immunohistochemistry (IHC) staining. The sums of scores for Lc, Rm, and Rc sections were shown in the table and shown as dot plots in FIG. 5-6.

FIG. 35 shows lung histopathology of antibody-treated and SARS-COV-2 challenged cynomolgus macaques. (FIG. 35A) Representative images of hematoxylin and eosin (H&E) staining and SARS-COV-2 antigen immunohistochemistry (IHC) staining from each group. All images were taken at 10× magnification. The images in this presentation are representative of the average severity of pathologic processes observed and recorded during microscopic evaluation. Red arrows indicate SARS-COV-2 infection foci. (FIG. 35B) Following microscopic evaluation of DH1052, 1 animal (BB536A) out of 5 animals in this group exhibited histologic features that was substantially more severe than the rest of the cohort and may suggest some degree of antibody-mediated disease enhancement. The features were characterized by prominent perivascular mononuclear inflammation (*) and a substantial amount of perivascular and alveolar edema (fluid; X). These findings suggest a vaso-centric process with some degree of altered vascular permeability. The remaining 4 animals in DH1052 group had inflammatory changes that ranged from minimal to moderate severity and more infiltrates were mixed and predominantly polymorphonuclear with lesser mononuclear cell involvement and present in the alveolar spaces.

FIG. 36 shows graphs of representative data. SARS-COV-2 total viral RNA and subgenomic RNA (sgRNA) in non-human primates that treated with RBD or NTD NAbs and infected with SARS-COV-2 (Related to FIGS. 5 and 6). Cynomolgus macaques (n=5 per group) were infused with RBD or NTD neutralizing antibodies 3 days before 105 PFU of SARS-COV-2 challenge. An irrelevant human antibody CH65 was used as a negative control. Viral load including viral RNA and subgenomic RNA (sgRNA) were measured on the indicated pre-challenge and post-challenge timepoints. Statistical significance in all the panels were determined using Wilcoxon rank sum exact test (ns, no significance, *P<0.05, ** P<0.001, *** P<0.0001). (FIGS. 36A,B) SARS-COV-2 (FIG. 36B) envelope gene (E gene) sgRNA and (FIG. 36C) nucleocapsid gene (N gene) sgRNA in nasal wash samples on Day 2 and Day 4 post challenge. (FIG. 36C) SARS-COV-2 E gene total (genomic+subgenomic) viral RNA in BAL samples on Day 2 and Day 4 post challenge. (FIG. 36D) SARS-COV-2 E gene total (genomic+subgenomic) viral RNA in nasal swab samples on Day 2 and Day 4 post challenge. (FIG. 36E) SARS-COV-2 E gene total (genomic+subgenomic) viral RNA in nasal wash samples on Day 2 and Day 4 post challenge.

FIG. 37 shows schematics and tables for cross-neutralizing RBD antibodies against SARS-COV-1 and SARS-related bat WIV1-CoV. (FIG. 37A) Maximum likelihood tree of Spike amino acid sequences for SARS-related group 2B and group 2C coronaviruses. (FIGS. 37B) Monoclonal RBD, NTD and S2 antibody ELISA binding titer for soluble S protein ectodomains from human and animal SARS-related coronaviruses, human circulating coronaviruses and

MERS. Titers are log area-under-the-curve (AUC). (FIG. 37C) Summary of cross-neutralizing antibodies against SARS-COV and/or bat WIV1-CoV.

FIG. 38 shows tables of ELISA screening of IgH/L transient transfection products recovered from COVID-19 and SARS donors. 38A contain data derived from SARS-Cov-1 donor memory B cells and show gene analysis data and reactivity with proteins, respectively. The antibodies were not reactive with MERS-COV, Coronavirus spike S1+S2 (Baculovirus-Insect Cells, His), MERS-COV Coronavirus spike S2 (Baculovirus-Insect Cells, His), Recombinant MERS-COV Spike/S1 Protein (S1 subunit, aa 1-725. His), MERS-COV Coronavirus spike RBD (Baculovirus-Insect Cells, His), Candida Albicans, biotin-Man9 (Strep), strep only control (superblock) subtracted, and Strep only control (Glycan) subtracted. 38B contains data derived from SARS-COV-2 donor plasmablasts. 38C contain data derived from SARS-COV-2 donor memory B cells.

FIG. 39 shows a table of COVID-19 RBD neutralizing antibodies (n=44).

FIG. 40 shows a table of COVID-19 RBD non-neutralizing antibodies (n=37).

FIG. 41 shows a table of COVID-19 NTD neutralizing antibodies (n=10).

FIG. 42 shows a table of COVID-19 NTD non-neutralizing antibodies (n=31).

FIG. 43 shows a table of COVID-19 S2 antibodies (n=65).

FIG. 44 shows a table of Cross-reactive COVID-19 antibodies (n=80).

FIG. 45 shows tables for all antibodies with Ig gene analysis. FIG. 45A shows COVID-19 RBD NAbs (n=44). FIG. 45B shows COVID-19 RBD non-NAbs (n=37). FIG. 45C shows COVID-19 NTD NAbs (n=10). FIG. 45D shows COVID-19 NTD non-Abs (n=31).

FIG. 45E shows COVID-19 S2 Abs (n=65). FIG. 45F show Cross-reactive Abs (n=80).

FIGS. 46A-C show amino acid and nucleic acid sequences of antibody DH1159.2. (A). In (A) Vh and VI are represented by the grey shaded sequences. CDRs are underlined. FIG. 26B-C show nucleic acid sequences of Vh and VI chains of antibody DH1073 and antibody DH1235 (B), and amino acid sequences of Vh and VI chains of antibody DH1073 and antibody DH1235 (C). In all panels CDRs per IMGT are underlined. Sequence ID numbers are shown in the figure.

FIG. 47 shows (A) clinical symptom score, (B) SARS-COV-2 viral load in nasal swab material and (C) serum antibody response for Donor ID #450905. Donor selected for cell sorting and antibody isolation.

FIG. 48 shows ELISA binding assays with transiently expressed antibody which demonstrates that DH1042 is SARS-COV-2 RBD specific.

FIG. 49 shows NESEM model of DH1042 Fab complexed with SARS-COV-2 spike trimer (A). The DH1042 putative epitope is the loop formed by residues 481-491 in the RBD of the spike (B) and (C). Red=epitope footprint within RBD, Blue highlight=secondary contacts.

FIGS. 50A-C show percent neutralization curves for A) SARS-COV-2 G614 pseudovirus, B) SARS-COV-2 D614 PRNT (Duke Regional Biocontainment Laboratory) and C) SARS-COV-2 D614 PRNT Bioqual vendor lab). DH1042 reaches 100% inhibition in all 3 assays.

FIG. 51 shows SPR kinetic response of DH1042 (Fab) binding to a titration of SARS-CoV-2 spike (2-50 mM).

FIG. 52 shows SPR kinetic response of DH1042 binding to a titration of: A) FcgRI/CD64, B) FcgRIIA/CD32a, and C) FcgRIIIA/CD16a. DH1042 was captured on a CM5 chip. BSA used as negative control surface. Fcgamma Receptors used: rhFcγRI/CD64 Lot: MOM2118911 MW: 31.4 kDa; rhFcγRIIA/CD32a Lot: MAC1717021 MW: 22 kDa; rhFcγRIIIA/CD16a Lot: QMI1919121 MW: 22.6 kDa, rhFcg Receptor Fab kinetics method: High performance injection with a flow rate of 50 uL/min, Injection duration was 300s, dissociation duration was 600s. Chip regeneration: 1 injection of 12s Gly2.0 at flow rate of 50 uL/min. The running buffer was 1× PBS.

FIG. 53 shows Representative IFA staining of HEP-2 cells with DH1042, two isotype control antibodies and a positive control antibody.

FIG. 54 shows DH1042 Heavy and Light Chain plasmids run uncut on a 1% agarose gel.

FIG. 55 shows dsRNA slot blot. Sample load (200 ng) versus blank. New England Bio (NEB) custom dsRNA ladder. Slots 1, 2, 5, and 6 are not relevant to DH1042.

FIG. 56 shows Lipid Nanoparticle (LNP) encapsulation analytical report for DH1042 (Ab026103) mRNA for pre-clinical studies.

FIG. 57 shows Pharmacokinetics of DH1042 when delivered as recombinant protein via 2 routes IV (A) and IM (B) to Tg32 mice, at two different doses.

FIG. 58 shows Pharmacokinetics of DH1042 when delivered as mRNA-LNP to Tg32 mice, at three different doses.

FIG. 59 shows DH1042 mRNA-LNP1 Expression and challenge studies in Hamsters. (A-D) IV administration of DH1042 mRNA-LNP produces functional antibody in serum with a 6.7 day half-life in hamsters. In serum (A-B), Thalf is 6.7 days and Tmax is 1.2 days. C-D shows lung. (E-G) Hamster SARS-COV-2 Challenge Study, PrEP with DH1042 mRNA-LNP1 (IV; 0.1. 0.5, 1.0 mg/kg)-WA1 Strain, and weight change. IV administration of DH1042 mRNA-LNP protects hamsters from challenge with wt SARS-Cov-2.

FIG. 60 shows DH1042 Ab protein protects/reduces viral load in SARS-COV-2 aged mouse challenge models. A) Pre exposure model timeline and Day 4 post challenge viral load as determined by plaque assay. B) Post exposure treatment model timeline and Day 4 post infection viral load as determined by plaque assay.

FIG. 61 shows DH1042 mRNA-LNP protects/reduces viral load in SARS-COV-2 hamster challenge model. Pre exposure model timeline, weight loss and in life oral swab post challenge viral load.

FIGS. 62A-B shows DH1041 Ab protein protects/reduces viral load in SARS-COV-2 aged mouse challenge models. A) Pre exposure model timeline and Day 2 post challenge viral load as determined by plaque assay. B) Post exposure treatment model timeline and Day 2 post infection viral load as determined by plaque assay.

FIGS. 63A-D show DH1041 and DH1043 mAb protein protect/reduce viral load in SARS-COV-2 NHP challenge model. A) Timeline of pre-exposure protection model and viral load sampling. B) Serum antibody levels on day of challenge. C) sgRNA E gene levels in day 2 and 4 BAL and nasal wash samples. D) sgRNA N gene levels in day 2 and 4 BAL and nasal wash samples.

FIG. 64 shows SARS-COV-2 spike(S) protein ectodomains for characterizing structures and antigenicity of S protein variants. Domain architecture of the SARS-COV-2 spike protomer. The SI subunit contains a signal sequence (SS), the NTD (N-terminal domain, pale green), N2R (NTD-to-RBD linker, cyan), RBD (receptor-binding domain, red), SD1 and SD2 (subdomain 1 and 2, dark blue and orange) subdomains. The S2 subunit contains the FP (fusion peptide, dark green), HR1 (heptad repeat 1, yellow), CH (central helix, teal), CD (connector domain, purple) and HR2 (heptad repeat 2, grey) subdomains. The transmembrane domain™ and cytoplasmic tail (CT) have been truncated and replaced by a foldon trimerization sequence (3), an HRV3C cleavage site (HRV3C), a His-tag (His) and strep-tag (Strep). The D614G mutation is in the SD2 domain (yellow star, green contour). The S1/S2 furin cleavage site (RRAR) has been mutated to GSAS (blue lightning). The substitutions in each variant are indicated by blue stars. * A few ectodomain constructs were prepared on the B.1.351 spike backbone; these differed in their NTD mutations. Binding data for the other constructs, including the one representing the dominant circulating form [L18F,D80A,D215G,A242-244,K417N,E484K,N501Y,D614G,A701V] are shown in FIG. S2 and S3. The construct shown here was used for determining the cryo-EM structure. The “P.1-like” spike was prepared in the P.1 backbone but retained the K417N RBD substitution (instead of the K417T in the P.1 spike; see FIG. 64).

FIGS. 65A-J show binding of ACE2 receptor ectodomain (FIG. 65A), DH1041 (FIG. 65C), DH1043 (FIG. 65B) and DH1047 (FIG. 65D) which are RBD-directed antibodies, DH1050.1 (FIG. 65E), DH1050.2 (FIG. 65G) and DH1052 (FIG. 65F) (NTD-directed), DH1058 (FIG. 65H) and 2G12 (FIG. 651) (S2 directed) to spike variants measured by SPR. The bar graphs represent the response levels (RU) after 20 seconds of dissociation. Data shown are representative of two independent experiments. Multiple ectodomain constructs in the B.1.351 spike backbone were tested that differed in their NTD mutations. The 242-244 deletion is carried in most circulating B.1.351 variants, and the R246I mutation is important because it is included in many reagent panels as the B.1.351 representative, but rare in nature with a transient local appearance in a small number of cases in South Africa in late 2020. In a sampling window of GISAID between December 1-May 24, B.1.351 with R246I was found five times, a B. 1.351 form of spike that did not have the deletion at 242-244 was found 24 times, and most common baseline form of spike [L18F,D80A,D215G,D242-244,K417N,E484K,N501Y,D614G,A701V], which has spread globally, was found 2,736 times. Our data showed that the NTD 242-244 deletion and the R246I substitution both impact the binding of NTD-directed antibodies, with the 242-244 deletion having a greater effect. These NTD changes also affect the binding of RBD directed nAbs DH1041 and DH1043, where the R246I substitution has a greater deleterious effect than the 242-244 deletion. Figure S2J shows the identity of spike constructs tested for binding and represented by each bar in Figures S2A-S2I. In FIGS. 65A-I, in order of appearance from left to right, each bar shows binding to a spike variant construct labeled 1-15 as listed in FIG. 65J. For the SPR assay spike variants were captured on a Series S Strepavidin (SA) chip. Fabs were then injected at 200 nM with a contact time of 60s and a dissociation time of 120s (50 uL/min).

FIGS. 66A-C shows binding of ACE2 receptor ectodomain (FIG. 66A), DH1041 (FIG. 66A), DH1043 (FIG. 66A) and DH1047 (FIG. 66B) (RBD-directed), DH1050.1 (FIG. 66A) (NTD-directed), to spike variants measured by ELISA. In the ELISA assay, serially diluted spike protein was bound in individual wells of 384-well plates, which were previously coated with streptavidin. Proteins were incubated and washed, then antibodies at 10 ug/ml or ACE2 with a mouse Fc tag at 2 ug/ml were added. Antibodies were incubated, washed and binding detected with goat anti-human-HRP. Multiple ectodomain constructs representing the B.1.351 spike were tested that differed in their NTD mutations. The ELISA data mirror the SPR data and show that the NTD 242-244 deletion and the R246I substitution both impact the binding of NTD-directed antibodies, with the 242-244 deletion having a greater effect. These NTD amino acid substitutions also affected the binding of RBD directed nAbs DH1041 and DH1043, where the R246I substitution showed a greater deleterious effect than the 242-244 deletion. Data shown are representative of two independent experiments. FIG. 66C lists the spike construct used in the ELISA assay depicted in the last panel.

FIG. 67 shows representative schematics and graphs in the identification of broadly neutralizing antibodies. (FIG. 67A) The genetic relationships of ACE2 and non-ACE2 using Sarbecovirus receptor binding domains. SARS-COV-2 2AA MA is shown in purple, SARS-COV is shown in orange, WIV-1 is shown in pink, and RsSHC014 is shown in green. The neutralization activity against sarbecoviruses is shown for (FIG. 67B) DH1235, (FIG. 67C) DH1073, (FIG. 67D) DH1046, and (FIG. 67E) DH1047.

FIG. 68 the binding breadth and structural determinants of broad neutralization. The binding activity of cross-reactive antibodies against SARS-COV spike, SARS-COV-2 spike, SARS-COV-2 RBD, Pangolin GXP4L spike, RaTG13 spike, and RsSHC014 spike of (FIG. 68A) DH1235, (FIG. 68B) DH1073, (FIG. 68C) DH1046, and (FIG. 68D) DH1047. (FIG. 68E) Cryo-EM reconstruction of DH1047 Fab bound to SARS-COV spike shown in grey, with the underlying fitted model shown in cartoon representation. DH1047 is colored green, the RBD it is bound to is colored black with the Receptor Binding Motif within the RBD colored purple. Overlay of DH1047 bound to SARS-COV-1 and SARS-COV-2 (PDB ID: 7LDI) S proteins. Overlay was performed with the respective RBDs. DH1047 bound to SARS-COV and SARS-COV-2 spike is shown in green and salmon, respectively. ACE2 (yellow surface representation, PDB 6VW1) binding to RBD is sterically hindered by DH1047. The views in panels B and C are related by a ˜180° rotation about the vertical axis. (FIG. 68F) Sequence conservation within the DH1047 HCDR3 and LCDR3s among 23 sarbecoviruses.

FIGS. 69A-H Prevention and therapy of DH1047 against SARS-COV in aged mice. (FIG. 69A) SARS-COV mouse-adapted 15 (MA15) lung viral replication in the prophylactically treated (−12 hours before infection) mice with a control influenza mAb CH65 and the four broadly neutralizing antibodies DH1235, DH1073, DH1046, DH1047. Days post infection (dpi). Limit of detection (LoD). (FIG. 69B) % Starting weight of prophylactic (−12 hours before infection) and therapeutic (+12 hours after infection) treatment with DH1047 and control against SARS-COV MA15 in mice. (FIG. 69C) Lung viral replication of SARS-COV MA15 in mice treated prophylactically and therapeutically with DH1047 and control at 4 days post infection. (FIG. 69D) Macroscopic lung discoloration scores in mice treated with DH1047 and control prophylactically and therapeutically. (FIG. 69E) Lung pathology at day 4 post infection measured by acute lung injury (ALI) scores in mice treated with DH1047 and control prophylactically and therapeutically. (FIG. 69F) Lung pathology at day 4 post infection measured by diffuse alveolar damage (DAD) in mice treated prophylactically and therapeutically with DH1047 and control. (FIG. 69G) Pulmonary function as measured by whole body plethysmography (Buxco) in DH1047 and control mAb prophylactically and therapeutically treated mice. P values are from a 2-way ANOVA after Tukey's multiple comparisons test for the weight loss, and P values are from a 1-way ANOVA following Dunnett's multiple comparisons for the viral titer, and lung pathology readouts. (FIG. 69H) Percent survival in DH1047 prophylaxis, therapy, and control groups.

FIGS. 70A-D Prophylactic and therapeutic activity of DH1047 against SARS-related bat CoVs and the in vitro neutralization against the SARS-COV-2 variants. (FIG. 70A) Lung viral replication of WIV-1 in mice treated prophylactically and therapeutically with DH1047 and control at 2 days post infection. Days post infection (dpi). Limit of detection (LoD). (FIG. 70B) Lung viral replication of RsSHC014 in mice treated prophylactically and therapeutically with DH1047 and control at 2 days post infection. (FIG. 70C) Live virus neutralization of SARS-COV-2 D614G, B.1.1.7., and B.1.351 variants. (FIG. 70D) The comparison of the DH1047 neutralization activity against SARS-COV-2 variants in pseudovirus and live virus neutralization assays. P values are from a 2-way ANOVA after Tukey's multiple comparisons test for the weight loss, and P values are from a 1-way ANOVA following Dunnett's multiple comparisons for the viral titer, and lung pathology readouts.

FIGS. 71A-I Prophylaxis and therapy of DH1047 against SARS-COV-2 B.1.351 in mice. (FIG. 71A) % Starting weight of prophylactic (−12 hours before infection) and therapeutic (+12 hours after infection) treatment with DH1047 and control against SARS-COV-2 B.1.351 in mice. (FIG. 71B) Lung viral replication of SARS-COV-2 B.1.351 in mice treated prophylactically and therapeutically with DH1047 and control at 4 days post infection. (FIG. 71C) Macroscopic lung discoloration scores in mice treated with DH1047 and control prophylactically and therapeutically. (FIG. 71D) Lung pathology at day 4 post infection measured by acute lung injury (ALI) scores in mice treated with DH1047 and control prophylactically and therapeutically. (FIG. 71E) Lung pathology at day 4 post infection measured by diffuse alveolar damage (DAD) in mice treated prophylactically and therapeutically with DH1047 and control. P values are from a 2-way ANOVA after Tukey's multiple comparisons test for the weight loss, and P values are from a 1-way ANOVA following Dunnett's multiple comparisons for the viral titer, and lung pathology readouts. (FIG. 71F) Percent survival in DH1047 prophylaxis, therapy, and control groups. (FIG. 71G) % Starting weight in dose de-escalation DH1047 prophylaxis (−12 hours before infection) at 10, 5, and 1 mg/kg and control mAb. (FIG. 71H) Lung viral titers in control and DH1047-treated mice at 10, 5, and 1 mg/kg. (FIG. 711) Lung discoloration in control and DH1047-treated mice at the various mAb doses. (FIG. 71H) DH1047 binding relative to binding of other known antibody classes that bind the RBD. RBD is shown in black with the ACE2 footprint on the RBD colored yellow. DH1047 is shown in cartoon representation and colored green. The other antibodies are shown as transparent surfaces: C105 (pale cyan, Class 1, PDB ID: 6XCN and 6XCA), DH1041 (light blue, Class 2, PDB ID: 7LAA), S309 (wheat, Class 3, PDB ID: 6WS6 and 6WPT) and CR3022 (pink, Class 4, PDB ID: 6YLA).

FIG. 72 the neutralization activity of DH1047, ADG-2, and REGN 10933 against sarbecoviruses. (A) SARS-COV-2 B.1.351 (B) SARS-COV urbani (C) RsSHC014.

FIG. 73 the binding activity of cross-reactive antibodies against MERS-COV and human common-cold CoVs. The binding activity of four broadly neutralizing antibodies against SARS-CoV-2 NTD, MERS-COV spike, HCoV-OC43 spike, HCoV-NL63, and HCoV-229E shown for (A) DH1235, (B) DH1073, (C) DH1046, and (D) DH1047.

FIGS. 74A-74D Coronavirus cross-reactive antibodies exhibit distinct binding modes to SARS-COV-2 receptor binding domain. (FIG. 74A) Three-dimensional reconstruction from negative stain electron microscopic images of DH1073 (purple) in complex with Hexapro stabilized SARS-COV-2 Spike ectodomain (gray). On the left and center the map is shown in two orthogonal views. On the right the map is shown as a transparent surface, rigidly fit with a 1-RBD-up spike model, PDB ID: 6ACK. (FIG. 74B) Three-dimensional reconstruction of DH1235 (blue) in complex with Hexapro stabilized Spike ectodomain (gray). The rigidly fit 3-RBD-up spike model on the right was generated from PDB ID: 6ACK. Fit models in A and B were used to compare the binding of DH1073 and DH1235 to known RBD-directed nAbs. (FIG. 74C) Four orthogonal views of RBD binding by DH1073 (purple) and DH1235 (blue), compared to known RBD nAbs, shown as molecular surface: C105 (pale cyan, Class 1, PDG ID 6XCN and 6XCA), DH1041 (light blue, Class 2, PDB ID: 7LAA), S309 (wheat, Class 3, PDB ID: 7BEP), CR3022 (pink, Class 4, PDB ID: 6W41), and DH1047 (green, PDB ID: 7LD1). The last two views correspond to the same views shown in FIG. 3D in the main text. DH1073 overlaps with DH1235, C105 (Class 1) and CR3022 (Class 4). DH1235 overlaps DH1073, DH1047, C105 and CR3022. (FIG. 74D) Antibody competition for binding to SARS-COV-2 S stabilized with two prolines (S-2P) in surface plasmon resonance assays. The antibody used to capture S-2P is shown as the row title. The antibody flowed over the captured spike is shown as column titles. +, blocking;-, no blocking of binding.

FIG. 75 NSEM of DH1047 bound to bat RsSHC014 and SARS-COV spike ectodomains. (A) Representative 2D class averages of bat RsSHC014 2P spike ectodomain bound to DH1047 Fab. (B) Overlay of 3D reconstruction of DH1047 bound to bat RsSHC014 2P (grey) and SARS-CoV-2 HexaPro (purple) S ectodomains. (C) Representative 2D class averages of SARS-COV 2P spike ectodomain bound to DH1047 Fab (D) Overlay of 3D reconstruction of DH1047 bound to bat SARS-COV 2P (grey) and SARS-COV-2 HexaPro (purple) S ectodomains. The red boxes in panels A and C indicate the classes that show DH1047 Fab bound to spike.

FIGS. 76A-76H Cryo-EM data processing for the SARS-COV spike ectodomain bound to DH1047, Related to FIG. 2. (FIG. 76A) Representative cryo-EM micrograph. (FIG. 76B) Cryo-EM CTF fit. (FIG. 76C) Representative 2D class averages from Cryo-EM dataset. (FIG. 76D) Ab initio reconstruction. (FIG. 76E) Refined map. (FIG. 76F) Fourier shell correlation curve. (FIG. 76G) Refined cryo-EM map colored by local resolution. (FIG. 76H) Zoom-in images showing the SD1, NTD, HR1/CH and RBD/Fab contact regions in the structure. The cryo-EM map is shown as a blue mesh and the fitted model is in cartoon representation, with residues shown as stick.

FIG. 77 the affinity data of DH1047 against SARS-COV and RsSHC014 spikes.

Surface plasmon resonance (SPR) binding experiments of DH1047 against (A) SARS-COV-2 Toronto and (B) RsSHC014. Binding affinity measurements are shown in the tables and response units (RU) as a function of time in seconds(s) is shown for both SARS-COV and RsSHC014. SPR experiments were repeated twice.

FIG. 78 lung H+E staining of SARS-COV infected mice. Pathologic features of acute lung injury were scored using two separate tools: the American Thoracic Society Lung Injury Scoring (ATS ALI) system. Using this ATS ALI system, we created an aggregate score for the following features: neutrophils in the alveolar and interstitial space, hyaline membranes, proteinaceous debris filling the air spaces, and alveolar septal thickening. Three randomly chosen high power (×60) fields of diseased lung were assessed per mouse. Representative images are shown from vehicle and RDV-treated mice. All images were taken at the same magnification. The black bar indicates 100 um scale. (A) CH65 control prophylaxis. (B) CH65 therapy. (C) DH1047 prophylaxis. (D) DH1047 therapy.

FIGS. 79A-79B Comparison of DH1047 binding footprint to that of other RBD-directed Abs that bind overlapping epitopes. (FIG. 79A) RBD is shown in black with ACE2 binding footprint colored yellow. Binding footprints of antibodies DH1047 (PDB: 7LD1), CR3022 (PDB: 6YLA), EY6A (PDB: 6ZCZ), H014 (PDB: 7CAH), COVA1-16 (PDB: 7JMW) and S304 (PDB: 7JW0) are shown in green. (FIG. 79B) Overlay of DH1047 bound RBD to RBD bound to nanobodies VHH V (PDB: 7KN6), VHH W (PDB: 7KN7) and VHH-72 (PDB: 6WAQ)

FIG. 80A-C Binding of DH1047 mutants to the SARS-COV-2 2P S ectodomain. (FIG. 80A) Binding assays were performed by Biolayer Interferometry in an Octet RED384 (Forte Bio). (Top) Anti-human IgG biosensor tips were dipped into 200 μl of DH1047 WT or mutant proteins for 5 min followed by Imin in 1×PBS. Next, the tips were dipped into 100 nM of SARS-COV-2 2P S ectodomain and binding response was measured. (Bottom) Binding of DH1047 IgG and mutants to SARS-COV-2 2P S ectodomain normalized to the binding observed for DH1047 WT IgG (dotted line). (FIG. 80B) Binding of purified DH1047 WT IgG and selected mutants to SARS-CoV-2 2P S ectodomain measured by Surface Plasmon Resonance in a Biacore T200 (Cytivia). The spike was captured via its C-terminal TwinStreptactin tag on a SA chip. Binding to the antibodies was measured by flowing over 200 nM solutions of the IgG in HBS-EP+buffer. (FIG. 80C) Binding interface of DH1047 to RBD (grey). The DH1047 residues that were mutated are shown as sticks and labeled.

FIGS. 81A-81B DH1047 and ADG-2 binds the RBD of SARS-Cov and SARS-COV-2 spike ectodomains using a similar footprint. (FIG. 81A) Cartoon representation of DH1047 (colored in pale green) bound to the RBD (grey surface, ACE2 binding site in yellow) of SARS-CoV S ectodomain and ADG-2 (cyan) bound to SARS-COV-2 S ectodomain. The homologous Fab ADI-19425 (PDB 6APC) was docked in the ADG-2 cryo-EM map (EMD-23160) to generate the model. (FIG. 81B) DH1047 and ADG-2 bind partially overlapping binding sites on the RBD. (FIG. 81C) Comparison of the therapeutic activity of DH1047, ADG-2, and control mAb in aged mice infected with SARS-COV-2 B.1.351 including % starting weight, lung viral replication titers, and lung pathology.

FIG. 82 monoclonal antibody neutralization screen against SARS-COV-2 2AA MA, SARS-COV, WIV-1, and RsSHC014.

FIG. 83 cryo-EM data collection and refinements statistics.

FIG. 84 immunogenetic characteristics of broadly cross-reactive mAbs.

FIG. 85 shows monoclonal antibody neutralization of different variants of SARS-COV-2 pseudovirus. Neutralization titers are shown as IC50 in micrograms per milliliter. The heatmap is color-coded by neutralization potency with potency increasing from green to red.

FIGS. 86A-B show binding breadth of SARS-COV-2 neutralizing receptor binding domain (RBD) antibodies determined by biolayer interferometry. N-terminal domain and S2 antibodies were used as negative controls. Bars in (A) show binding to SARS-COV-2 HexaPro, SARS-COV-2 Wuhan 1 RBD, SARS-COV-2 Beta RBD, SARS-COV-2 Delta, HIV-1 envelope. In (B) bars show binding to SARS-COV-2 kappa RBD SARS-COV-2 Wuhan 1 RBD, and Alphacoronavirus HuPn-2018 RBD, HIV-1 envelope in order of appearance. HuPn-2018 RBD was used to assess antibody reactivity with a new alphacoronavirus purported to infect humans in Malaysia. S_Hexapro is a SARS-COV-2 stabilized spike ectodomain. In both (A) and (B) HIV-1 envelope was used as a negative control.

FIGS. 87A-B show summary of immunogenetics, Vh and VI names and mutation frequency of select antibodies including DH1221-1226, DH1047, DH1073, DH1235 and DH1236. Sequences are shown in FIGS. 20-23, and FIG. 46.

### DETAILED DESCRIPTION

The present invention relates to antibodies and antigen binding fragments thereof, including recombinant and/or derivative forms that bind to coronavirus spike protein. In some embodiments, the antibodies or fragments bind specifically to epitopes on spike protein. In non-limiting embodiments, the antibodies are RBD binding antibodies. In some non-limiting embodiments the RBD binding antibodies are ACE-2 blocking neutralizing antibodies. In some non-limiting embodiments the RBD binding antibodies are non-ACE-2 blocking neutralizing antibodies. In some non-limiting embodiments the RBD binding antibodies are SARS-COV-2 neutralizing antibodies. In some non-limiting embodiments the RBD binding antibodies are cross reactive with SARS-CoV-1. In other non-limiting embodiments, the antibodies are NTD binding antibodies. In some non-limiting embodiments, the NTD antibodies are neutralizing antibodies. In some non-limiting embodiments, the NTD antibodies are ADE

### Recombinant Antibodies

Recombinant antibodies of the invention include antibodies derived from rearranged VDJ variable heavy chain (VH) and/or rearranged VJ variable light chain (VL) sequences from individual or clonal cells that express an antibody that specifically binds to coronavirus spike protein, and optionally are neutralizing. Antibodies are described in the accompanying examples, figures, and tables, and the invention includes antibodies comprising CDR sequences contained with the VH and VL amino acid sequences described herein. In certain embodiments, the invention provides monoclonal antibodies. In certain embodiments the monoclonal antibodies are produced by a clone of B-lymphocytes. In certain embodiments the monoclonal antibody is recombinant and is produced by a host cell into which an expression vector(s) encoding the antibody, or fragment thereof, has been transfected.

Methods for obtaining rearranged heavy and light chain sequences are well known in the art and often involve amplification-based-cloning and sequencing. Standard techniques of molecular biology may be used to prepare DNA sequences encoding the antibodies or antibody fragments of the present invention. DNA sequences can be synthesized completely or in part using oligonucleotide synthesis techniques. Site-directed mutagenesis and polymerase chain reaction (PCR) techniques may be used as appropriate.

The invention encompasses antibodies which are 99%, 98%, 97%, 96%, 95%, 94%, 93%, 92%, 91%, 90%, 89%, 88%, 87%, 86%, 85%, 84%, 83%, 82%, 81%, 80%, 75% identical to the VH and/or VL variable domain amino acid sequences of the antibodies described herein in the Figures or Tables 3-6, 10 or 11. Further, the invention encompasses variants having one or mutations (99% et seq, per herein) as compared to the sequences of the antibodies of the Figures or Tables 3-6, 10 or 11 with one or more of the additional requirements: (1) the variant maintains antigen binding specificity to the spike protein, and in some embodiments, maintains the ability to specifically bind an epitope that includes RBD, NTD, or S2, (2) the variant does not have a decrease in binding affinity or avidity that is more than 10-fold, 5-fold, 2-fold, or 1-fold than the corresponding antibody of the Figures or Tables 3-6, 10 or 11, (3) the variant has a binding affinity or avidity that is an improvement of more than 100-fold, 10-fold, 5-fold, 2-fold, or 1-fold more than the corresponding antibody of the Figures or Table 3-6, (4) the variant does not have a decrease in promoting neutralization that is more than 10-fold, 5-fold, 2-fold, or 1-fold as compared to the corresponding antibody of the Figures or Tables 3-6, 10 or 11, (5) the variant has an increase in neutralization that is 10-fold, 5-fold, 2-fold, or 1-fold more as compared to the corresponding antibody of the Figures or Tables, and (6) as compared to the antibodies listed in the Figures or Tables 3-6, 10 or 11, the variant has a V region with shared mutations compared to the germline, identical VDJ or VJ gene family usage, identical or the same or similar HCDR3 length, and the same VL and JLgene family usage. For example, one or more of these six requirements are applicable to any antibody, including fragments (see herein, Fab, Fv, et al.) or portions (VH, VL, one or more CDRs from a VH/VL pair) thereof, derived from the antibodies listed in Tables 3-6, 10 or 11 or the Figures. Various figure provide non-limiting embodiments of nucleic acids and plasmids for expression of mRNA encoded antibodies.

With respect to the antibodies listed in Tables 3-6, 10 or 11, their corresponding sequences are provided in at least FIGS. 20 and 22, where VH and VL portions are indicated. CDR regions are determined by any suitable method, e.g. but not limited by IMGT (www.imgt.org/IMGT_vquest). The boundaries of these CDR regions can be modified or substituted according to other CDR conventions as known in the art and as described herein.

Binding specificity can be determined by any suitable assay in the art, for example but not limited competition binding assays, epitope mapping, structural studies of antibodies, or fragments thereof, bound to target envelopes, etc.

Affinity can be measured, for example, by surface plasmon resonance. It is well-known in the art how to conduct SPR for measuring antibody affinity to an antigen. SPR affinity measurements can provide the affinity constant K_{D} of an antibody, which is based on the association rate constant kₒₙ divided by the disassociation rate constant k_{off}. Thus, in certain embodiments, comparing affinity between a variant and an antibody of the invention (e.g. Tables 3-6, 10 or 11) is based on K_{D}. In other embodiments, the comparison is based only on k_{off}. When comparing affinity between antibodies, the antibodies can have the same valency, i.e., Fab vs. Fab, scFv vs, scFv, IgG v. IgG, IgM v. IgM, etc. Thus, when the comparison is between antibodies that are not monovalent, then affinity is a measure of functional affinity. In the art, functional affinity covers the binding strength of a bi- or polyvalent antibody to antigens that present more than one copy of an epitope, because they are multimeric or conjugated in multiple copies to a solid phase, thus allowing cross-linking by the antibody. It is known in the art that a monovalent antibody fragment (e.g., Fab) provides a measure of intrinsic affinity irrespective of the density of antigens (SPR often immobilizes antigen on a solid substrate and the antibody is flowed over the substrate thereby allowing kinetic measurements of antibody association and disassociation rates).

Avidity can also be measured by SPR. Avidity can be quantitatively expressed, for example, by the ratio of K_{D} for a Fab over the multivalent form, e.g., IgG, IgM . . .

Potency can be measured, for example, by a virus inhibition pseudovirus assay or authentic virus Plaque Reduction Neutralization Test).

In certain embodiments, the invention provides antibodies with CDR amino acid sequences that are 99%, 98%, 97%, 96%, 95%, 94%, 93%, 92%, 91%, 90%, 89%, 88%, 87%, 86%, 85%, 84%, 83%, 82%, 81%, 80% identical to the CDR1, 2, and/or 3 of VH (also referred to as CDRH1, CDRH2, and CDRH3) and/or CDR1, 2, and/or 3 of VL (also referred to as CDRL1, CDRL2, and CDRL3) amino acid sequences of the antibodies of Tables 3-6, 10 or 11.

In certain embodiments, the invention provides antibodies with CDR amino acid sequences that are 99%, 98%, 97%, 96%, 95%, 94%, 93%, 92%, 91%, 90%, 89%, 88%, 87%, 86%, 85%, 84%, 83%, 82%, 81%, 80% identical to CDRs to an antibody of Table 3-6, 10 or 11, where each CDR can have a different percent identity. For example, the antibody has at least 99%, 98%, 97%, 96%, or 95% identity for all CDRs as compared to the CDRs of an antibody listed in Table 3-6 except HCDR3 and LCDR3, which can allow for a lower percent identity, for example, 99% to 80%, 99% to 85%, 99% to 90%, or 99% to 95%.

In certain embodiments, the invention provides antibodies which can tolerate a larger percent variation in the sequences outside of the VH and/VL sequences of the antibodies. In certain embodiments, the invention provides antibodies which are 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65% identical, wherein the identity is outside of the VH or VL regions, or the CDRs of the VH or VL chains of the antibodies described herein.

In some aspects, the antibody or antigen binding fragment thereof, comprises a heavy chain comprising at least one CDRH1, at least one CDRH2 and at least one CDRH3 and a light chain comprising at least one CDRL1, at least one CDRL2 and at least one CDRL3, wherein at least one CDR, comprises, consists essentially of or consists of an amino acid sequence according to any of the sequences of the antibodies described herein, or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional variation is 80%, 81%, 82%, 83%, 84%, 85%, 86%, 87%, 88%, 89%, 90%, 91%, 92%, 93%, 94%, 95%, 96%, 97%, 98% or 99% identity. As stated herein, the person of ordinary skill in the art can select from one or more CDR conventions to identify the boundaries of the CDR regions.

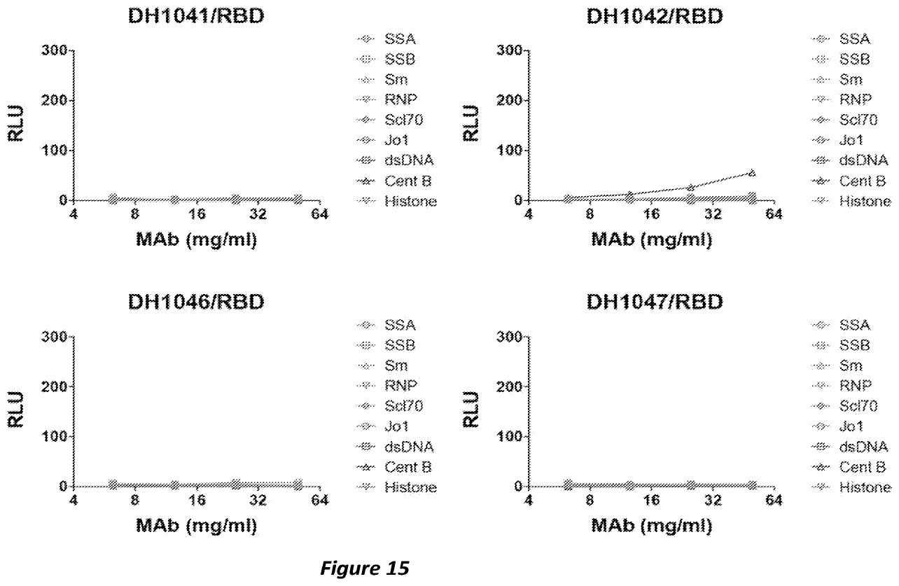

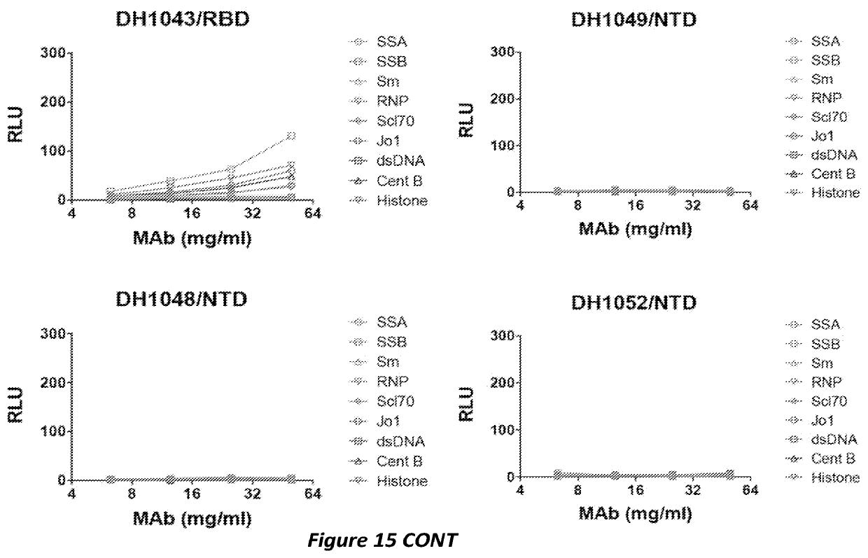

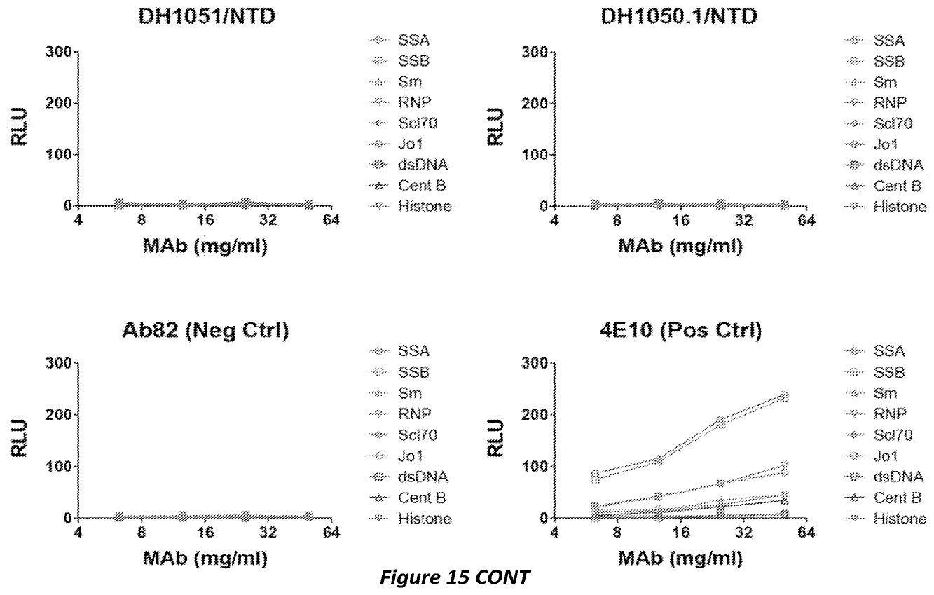

In some aspects, the antibody or antigen binding fragment thereof, comprises, consists essentially of or consists of a VH amino acid sequence or a VL amino acid sequence in FIG. 15 or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments the functional variation is 80%, 81%, 82%, 83%, 84%, 85%, 86%, 87%, 88%, 89%, 90%, 91%, 92%, 93%, 94%, 95%, 96%, 97%, 98% or 99% identity.

In some aspects, the antibody or antigen-binding fragment thereof, comprises, consists essentially of or consists of a VH amino acid sequence and/or a VL amino acid sequence according to FIG. 20-25, 46.

Furthermore, the invention provides antibodies that are affinity matured in vitro. The affinity of an antibody to its antigen target can be modulated by identifying mutations introduced into the variable region or into targeted sub-regions. For example, it is known in the art that one can sequentially introduce mutations through each of the CDRs, optimizing one at a time, or to focus on CDRH3 and CDRL3, or CDRH3 alone, because it often forms the majority of antigen contacts. Alternatively, it is known in the art how to simultaneously mutagenize all six CDRs by generating large-scale, high-throughput expression and screening assays, such as by antibody phage display. Antibody-antigen complex structural information can also be used to focus affinity maturation to a small number of residues in the antibody binding site.

Various algorithms for sequence alignment are known in the art. For example, the NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

### CDRs and Frameworks

In some embodiments, the CDRs of the antibodies and fragments of the invention are defined according to the IMGT scheme. IMGT-defined CDR regions have been highlighted/underlined in the nucleotide and amino acid sequences for each of the VH and VL variable regions of the antibodies of Table 3-6, 10 or 11. (See FIGS. 20, 22.) IMGT sequence analysis tools will identify CDR and framework regions in the nucleotide sequence and translated amino acid sequence. See www.imgt.org/IMGT_vquest/analysis

In some embodiments, CDR and framework regions can be identified based on other classical variable region numbering and definition schemes or conventions, including the Kabat, Chothia, Martin, and Aho schemes. The ANARCI (Antigen receptor Numbering And Receptor Classification; see http: opig.stats.ox.ac.uk webapps newsabdab sabpred anarci) online tool allows one to input amino acid sequences and to select an output with the IMGT, Kabat, Chothia, Martin, or AHo numbering scheme. With these numbering schemes, CDR and framework regions within the amino acid sequence can be identified. The person of ordinary skill is able to ascertain CDR and framework boundaries using one or more of several publicly available tools and guides.

Different methods of identifying CDRs are well known and described in the art (e.g. Kabat, Chothia, IMGT). Delineating CDRs by any one of these methods will result in CDRs with specific boundaries within a VH or VL sequence as listed herein. CDRs identified by any one of the methods are specific and well defined. See, for example, Martin, A. C., R, “Chapter3: Protein Sequence and Structure Analysis of Antibody Variable Domains,” Antibody Engineering, vol. 2 (2nd ed.), Springer-Verlag, Berlin Heidelberg pp. 33-51 (2010) (describing inter alia Kabat, Chothia, IMGT); and Munshaw, S, and Kepler, T. B., “SoDA2: a Hidden Markov Model approach for identification of immunoglobulin rearrangements,” Bioinformatics, vol. 26, No. 7, pp. 867-872 (February 2010) (describing SoDA2). Any of these methods for identifying CDRs may be used with the technology described herein.

For example, provided herein is a general, not limiting guide, for the CDR regions as based on different numbering schemes (see http: www.bioinf.org.uk abs info.html #cdrid). In the Table, any of the numbering schemes can be used for these CDR definitions, except the Contact CDR definition uses the Chothia or Martin (Enhanced Chothia) numbering.

TABLE 1
General Guide of Selected CDR Definitions

| | Kabat CDR | AbM CDR | Chothia CDR | Contact CDR | IMGT CDR |
|---|---|---|---|---|---|
| CDR Loop | Definition | Definition | Definition | Definition | Definition |
| CDRL1 | L24-L34 | L24-L34 | L24-L34 | L30-L36 | L27-L32 |
| CDRL2 | L50-L56 | L50-L56 | L50-L56 | L46-L55 | L50-L51 |
| CDRL3 | L89-L97 | L89-L97 | L89-L97 | L89-L96 | L89-L97 |
| CDRH1 | H31-H35B | H26-H35B | H26- | H30-H35B | H26-H35B |
| (Kabat | | | H32 . . . H34 | | |
| numbering) | | | | | |
| CDRH1 | H31-H35 | H26-H35 | H26-H32 | H30-H35 | H26-H33 |
| (Chothia | | | | | |
| numbering) | | | | | |
| CDRH2 | H50-H65 | H50-H58 | H52-H56 | H47-H58 | H51-H56 |
| CDRH3 | H95-H102 | H95-H102 | H95-H102 | H93-H101 | H93-H102 |

In the table herein, for CDRH1 Kabat numbering using the Chothia CDR definition, the boundary is H26 to H32 or H34 because the Kabat numbering convention varies between H32 and H34 depending on the length of the loop. This is because the Kabat numbering scheme places insertions at H35A and H35B. If neither H35A nor H35B is present, CDRH2 ends at H32. If only H35A is present, the loop ends at H33. If both H35A and H35B are present, the loop ends at H34.

Alternatively or in combination, one can examine amino acid sequences and identify CDR and framework regions according to the following alternative general guideline of Table AB herein, which is non-limiting.

TABLE 2
General Guide for Demarcating CDR Boundaries

| CDR | |
|---|---|
| Region | Guidelines |
| LCDR1 | Start: approx. residue 24 |
| | Residue before: always a Cys |
| | Residue after: always a Trp. Such as Trp-Tyr-Gln, but also, |
| | Trp-Leu-Gln, Trp-Phe-Gln, Trp-Tyr-Leu |
| | Length: 10 to 17 residues |
| LCDR2 | Start: always 16 residues after the end of L1. Residues before |
| | Ile-Tyr, but also, Val-Tyr, Ile-Lys, Ile-Phe |
| | Length: always 7 residues (except NEW (7FAB) which has a |
| | deletion in this region) |
| LCDR3 | Start: always 33 residues after end of L2 (except NEW (7FAB) |
| | which has the deletion at the end of CDR-L2). Residue before |
| | always Cys. Residues after always Phe-Gly-XXX-Gly |
| | Length |
| | 7 to 11 residues |
| HCDR1 | Start |
| | Approx. residue 26 (always 4 after a Cys) [Chothia/AbM |
| | definition]; |
| | Kabat definition starts 5 residues later |
| | Residues before |
| | always Cys-XXX-XXX-XXX |
| | Residues after |
| | always a Trp. Such as Trp-Val, but also, Trp-Ile, Trp-Ala |
| | Length |
| | 10 to 12 residues [AbM definition]; |
| | Chothia definition excludes the last 4 residues |
| HCDR2 | Start |
| | always 15 residues after the end of Kabat/AbM definition) of |
| | CDR-H1 |
| | Residues before |
| | such as Leu-Glu-Trp-Ile-Gly, but a number of variations |
| | Residues after |
| | Lys/Arg-Leu/Ile/Val/Phe/Thr/Ala-Thr/Ser/Ile/Ala |
| | Length |
| | Kabat definition 16 to 19 residues; |
| | AbM (and recent Chothia) definition ends 7 residues earlier |
| HCDR3 | Start |
| | always 33 residues after end of CDR-H2 (always 2 after a Cys) |
| | Residues before |
| | always Cys-XXX-XXX (such as Cys-Ala-Arg) |
| | Residues after |
| | always Trp-Gly-XXX-Gly |
| | Length |
| | 3 to 25(!) residues |

In Figures showing sequences of the inventive antibodies, CDR boundaries can be defined according to any method, e.g. the IMGT scheme. Because framework (FR) regions constitute all of the variable domain sequence outside of the CDRs, once CDR boundaries are identified, framework regions are necessarily identified. The convention within the art is to label the framework regions as FR1 (sequence before CDR1), FR2 (sequence between CDR1 and CDR2), FR3 (sequence between CDR2 and CDR3), and FR4 (sequence after CDR3).

CDR and framework regions can also be demarcated using other numbering schemes and CDR definitions. The ABnum tool numbers the amino acid sequences of variable domains according to a large and regularly updated database called Abysis, which takes into account insertions of variable lengths and integrates sequences from Kabat, IMGT, and the PDB databases. The Honneger scheme is based on structural alignments of the 3D structures of immunoglobulin variable regions and allows one to define structurally conserved Ca positions and deduction of appropriate framework regions and CDR lengths (Honegger and Plückthun, J. Mol. Biol., 2001, 309:657-70). Similarly, Ofran et al, used a multiple structural alignment approach to identify the antigen binding residues of the variable regions called “Antigen Binding Regions (ABRs)” (Ofran et al., J. Imunol., 2008, 181:6230-5). ABRs can be identified using the Paratome online tool that identifies ABR by comparing the antibody sequence with a set of antibody-antigen structural complexes (Kunik et al., Nucleic Acids Res., 2012, 40:521-4). Another alternative tool is the proABC software, which estimates the probabilities for each residue to form an interaction with the antigen (Olimpieri et al., Bioinformatics, 2013, 29:2285-91).

In some embodiments, the CDRs of the antibodies of the invention are defined by the scheme or tool that provides the broadest or longest CDR sequence. In some embodiments, the CDRs are defined by a combination of schemes or tools that provides the broadest/longest CDRs. For example, from the Table of CDR Definitions herein, CDRL1 will be L24-L36, CDRL2 will be L46-L56, CDR3 will be L89-L97, CDRH1 will be H26-H35/H35B, CDRH2 will be H47-H65, and CDRH3 will be H93-H102. In some embodiments, the CDRs are defined by the Anticalign software, which automatically identifies all hypervariable and framework regions in experimentally elucidated antibody sequences from an algorithm based on rules from the Kabat and Chothia conventions (Jarasch et al., Proteins Struct. Funct. Bioinforma, 2017, 85:65-71). In some embodiments, the CDRs are defined by a combination of the Kabat, IMGT, and Chothia CDR definitions. In some embodiments, the CDRs are defined by the Martin scheme in combination with the Kabat and IMGT schemes. In some embodiments, the CDRs are defined by a combination of the Martin and Honneger schemes. In some embodiments, the CDRs comprise the ABR residues identified by the Paratome tool.

The complete human immunoglobulin germline gene loci and alleles are available in the Immunogenetics Database (IMGT). Skilled artisan can readily determine the V, D, and/or J heavy and/or light sequences of various embodiments of antibodies of the invention of fragments thereof.

### Fragments and Other Engineered Antibody Forms

In certain embodiments the invention provides antibody fragments, which have the binding specificity and/or properties of the inventive antibodies. Recombinant fragments of the antibodies can be obtained by cloning and expression of part of the sequences of the heavy or light chains.

Antibody “fragments” include Fab, Fab′, F(ab′)2, F (ab) c, diabodies, Dabs, nanobodies, and Fv fragments. Also included are heavy or light chain monomers and dimers, single domain heavy chain antibodies, single domain light chain antibodies, (a single domain antibody, sdAb, is also referred to in the art as a nanobody) as well as single chain antibodies, e.g., single chain Fv in which the heavy and light chain variable domains are joined by a peptide linker. (See, e.g., Bird et al., Science 242:423-426, 1988; Huston et al., Proc. Natl. Acad. Sci. USA 85:5879-5883, 1988; McCafferty et al., Nature 348:552-554, 1990). Optionally, a cleavage site can be included in a linker, such as a furin cleavage site.

A recombinant antibody can also comprise a heavy chain variable domain from one antibody and a light chain variable domain from a different antibody. Further, the invention encompasses chimeric antigen receptors (CARs; chimeric T cell receptors) engineered from the variable domains of antibodies. (Chow et al, Adv. Exp. Biol. Med., 2012, 746:121-41). The Chimeric Antigen Receptor (CAR) consists of an antibody-derived targeting domain (including fragments such as scFv or Fab) fused with T-cell signaling domains that, when expressed by a T-cell, endows the T-cell with antigen specificity determined by the targeting domain of the CAR.

### Fc Domains

Whether full-length, or fragments engineered to have a Fc domain, (or particular constant domain portions thereof), the antibodies of the invention can be of any isotype or have any Fc (or portion thereof) of any isotype. It is well-known in the art how to engineer Fc domains or portions together with antibody fragments.

In certain embodiments, the antibodies of the invention can be used as IgG1, IgG2, IgG3, IgG4, whole IgG1 or IgG3s, whole monomeric IgAs, dimeric IgAs, secretory IgAs, IgMs as monomeric, pentameric, hexameric, or other polymer forms of IgM. The class of an antibody comprising the VH and VL chains described herein can be specifically switched to a different class of antibody by methods known in the art.

In some embodiments, the nucleic acid encoding the VH and VL can encode an Fc domain (immunoadhesin). The Fc domain can be an IgA, IgM or IgG Fc domain. The Fc domain can be an optimized Fc domain, as described in U.S. Published patent application No. 20100093979, incorporated herein by reference. In one example, the immunoadhesin is an IgG1 Fc. In one example, the immunoadhesin is an IgG3 Fc.

In one embodiment, the IgG constant region comprises the LS mutation. Embodiments also comprise additional variants of the Fc portion of the antibody. See Maeda et al. MAbs. 2017 July; 9 (5): 844-853. Published online 2017 Apr. 7, PMID: 28387635; see also Booth et al. MAbs. 2018 October; 10 (7): 1098-1110. Published online 2018 Jul. 26, doi: 10.1080/19420862.2018.1490119.

In certain embodiments the antibodies comprise amino acid alterations, or combinations thereof, for example in the Fc region outside of epitope binding, which alterations can improve their properties. Various Fc modifications are known in the art.

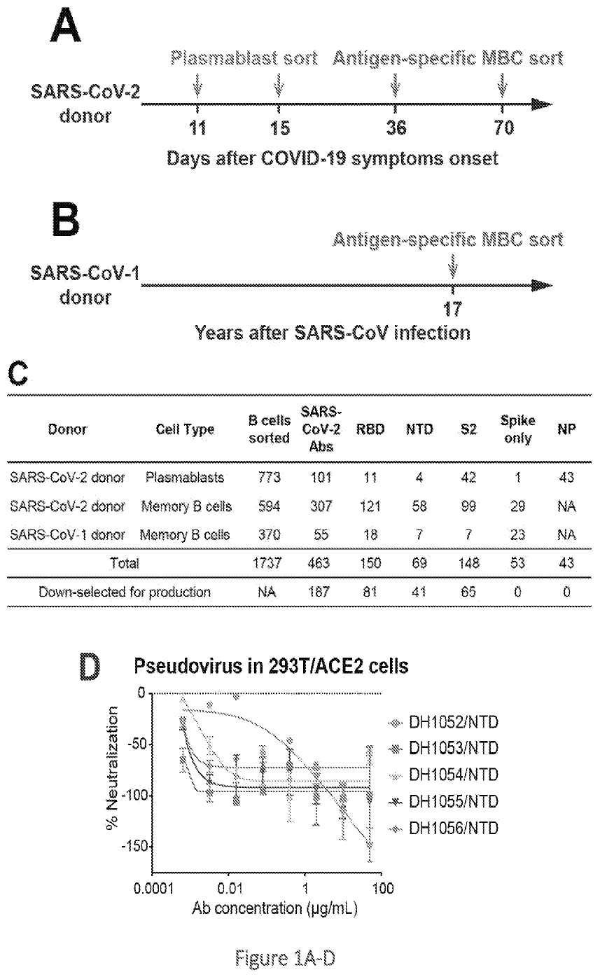

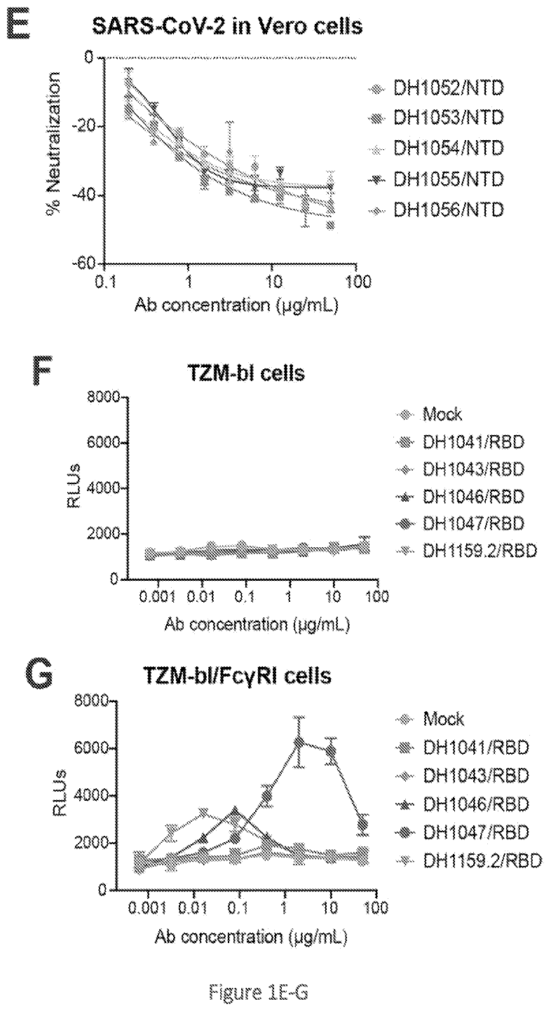

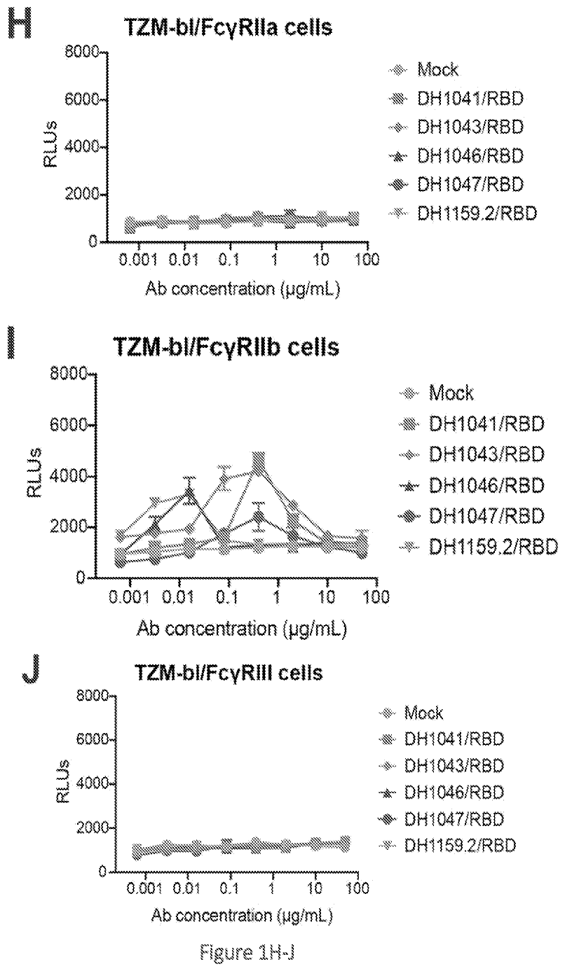

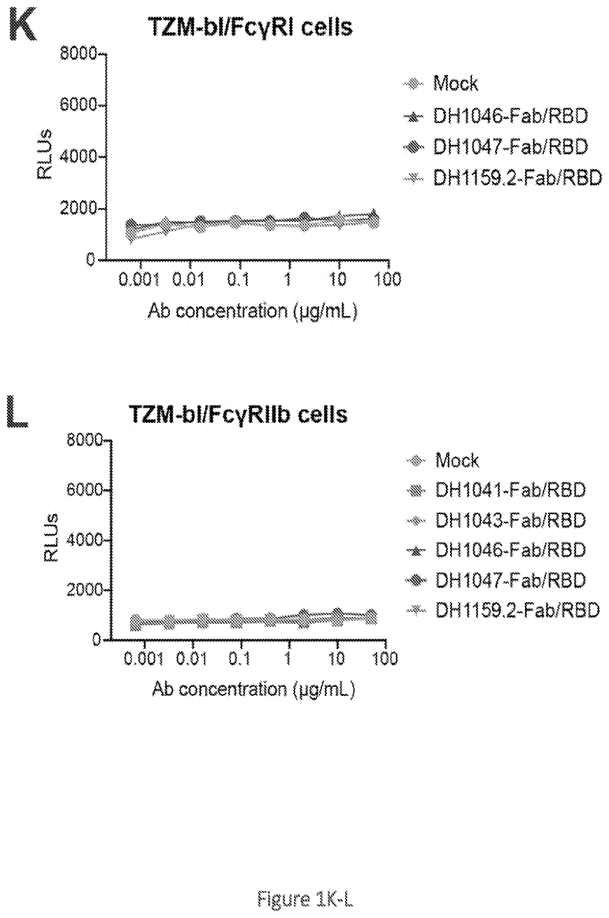

Embodiments comprise antibodies comprising mutations that affect neonatal Fc receptor (FcRn) binding, antibody half-life, and localization and persistence of antibodies at mucosal sites. See e.g. Ko S Y et al., Nature 514:642-45, 2014, at FIG. 1a and citations therein; Kuo, T. and Averson, V., mAbs 3 (5): 422-430, 2011, at Table 1, US Pub 20110081347 (an aspartic acid at Kabat residue 288 and/or a lysine at Kabat residue 435), US Pub 20150152183 for various Fc region mutation, incorporated by reference in their entirety.

In certain embodiments, the antibodies comprise AAAA substitution in and around the Fc region of the antibody that has been reported to enhance ADCC via NK cells (AAA mutations) containing the Fc region aa of S298A as well as E333A and K334A (Shields R I et al JBC, 276:6591-6604, 2001) and the 4th A (N434A) is to enhance FcR neonatal mediated transport of the IgG to mucosal sites (Shields R I et al, ibid).

Other antibody mutations have been reported to improve antibody half-life or function or both and can be incorporated in sequences of the antibodies. These include the DLE set of mutations (Romain G, et al. Blood 124:3241, 2014), the LS mutations M428L/N434S, alone or in a combination with other Fc region mutations, (Ko S Y et al. Nature 514:642-45, 2014, at FIG. 1a and citations therein; Zlevsky et al., Nature Biotechnology, 28 (2): 157-159, 2010; US Pub 20150152183); the YTE Fc mutations (Robbie G et al Antimicrobial Agents and Chemotherapy 12:6147-53, 2013) as well as other engineered mutations to the antibody such as QL mutations, IHH mutations (Ko S Y et al. Nature 514:642-45, 2014, at FIG. 1a and relevant citations; See also Rudicell R et al. J. Virol 88:12669-82, 201).

In some embodiments, modifications, such as but not limited to antibody fucosylation, may affect interaction with Fc receptors (See e.g. Moldt, et al. JVI 86 (11): 66189-6196, 2012). In some embodiments, the antibodies can comprise modifications, for example but not limited to glycosylation, which reduce or eliminate polyreactivity of an antibody. See e.g. Chuang, et al. Protein Science 24:1019-1030, 2015.

In some embodiments the antibodies can comprise modifications in the Fc domain such that the Fc domain exhibits, as compared to an unmodified Fc domain enhanced antibody dependent cell mediated cytotoxicity (ADCC); increased binding to Fc.gamma.RIIA or to Fc.gamma.RIIIA; decreased binding to Fc.gamma.RIIB; or increased binding to Fc.gamma.RIIB. See e.g. US Pub 20140328836.

### Multivalent and Multispecific Antibodies

In certain embodiments the invention provides a multivalent and multispecific antibody. A multivalent antibody has at least two antigen-binding sites, i.e., at least two heavy/light chain pairs, or fragments thereof. When the heavy/light pairs of a multivalent antibody bind to different epitopes, whether on the same antigen or on different antigens, the antibody is considered to be multispecific. Antibody fragments may impart monovalent or multivalent interactions and be contained in a variety of structures as described herein. For instance, monovalent scFv molecules may be synthesized to create a bivalent diabody, a trivalent “triabody” or a tetravalent “tetrabody.” The scFv molecules may include a domain of the Fc region resulting in bivalent minibodies. In addition, the sequences of the invention may be a component of multispecific molecules in which the sequences of the invention target the epitopes of the invention and other regions of the molecule bind to other targets. Exemplary molecules include, but are not limited to, bispecific Fab2, trispecific Fab3, bispecific scFv, and diabodies (Holliger and Hudson, 2005, Nature Biotechnology 9:1126-1136).

In some embodiments, multivalent but not multispecific antibodies are provided, where the multivalent antibody comprises multiple identical VH/VL pairs, or the CDRs from the VH and a VL pairs. This type of multispecific antibody will serve to improve the avidity of an antibody. For example, a tetramer can comprise four identical scFvs where the scFv is based on the VH/VL pair from an antibody of Table 3-6, 10 or 11.

In some embodiments, multivalent but not multispecific antibodies comprise multiple VH/VL pairs (or the CDRs from the pairs) where each pair binds to an overlapping epitope. In some embodiments, multivalent but not multispecific antibodies comprise multiple VH/VL pairs (or the CDRs from the pairs) where each pair binds to an non-overlapping epitope. Determining overlapping epitopes can be conducted, for example, by structural analysis of the antibodies and competitive binding assays as known in the art.

In some embodiments, multispecific antibodies comprise multiple VH/VL pairs (or the CDRs from the pairs) where each pair binds to a distinct epitope (not overlapping) spike protein.

In some embodiments, multispecific antibodies or fragments of the invention comprise at least a VH and a VL pair from Tables 3-6, 10 or 11, or the CDRs from the VH and a VL pair, in order to provide the multispecific antibody with binding specificity to the spike protein. The multispecific antibody can have one or more additional binding specificities by further comprising antibody binding site fragments from antibodies that bind to different antigens.

In certain embodiments the invention provides a bispecific antibody. A bispecific or bifunctional/dual targeting antibody is an artificial hybrid antibody having two different heavy/light chain pairs and two different binding sites (see, e.g., Romain Rouet & Daniel Christ “Bispecific antibodies with native chain structure” Nature Biotechnology 32, 136-137 (2014); Garber “Bispecific antibodies rise again” Nature Reviews Drug Discovery 13, 799-801 (2014), FIG. 1a; Byrne et al. “A tale of two specificities: bispecific antibodies for therapeutic and diagnostic applications” Trends in Biotechnology, Volume 31, Issue 11, November 2013, Pages 621-632 Songsivilai and Lachmann, Clin. Exp. Immunol., 79:315-321 (1990); Kostelny et al., J. Immunol. 148:1547-53 (1992) (and references therein)). In certain embodiments the bispecific antibody is a whole antibody of any isotype. In other embodiments it is a bispecific fragment, for example but not limited to F (ab) 2 fragment. In some embodiments, the bispecific antibodies do not include Fc portion, which makes these diabodies relatively small in size and easy to penetrate tissues.

Non-limiting examples of multispecific antibodies also include: (1) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig.TM.) Molecule, In: Antibody Engineering, Springer Berlin Heidelberg (2010)); (2) a Tandab, which is a fusion of two single chain diabodies resulting in a tetravalent bispecific antibody that has two binding sites for each of the target antigens; (3) a flexibody, which is a combination of scFvs with a diabody resulting in a multivalent molecule; (4) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (5) a so-called Scorpion molecule, comprising, e.g., two scFvs fused to both termini of a human Fc-region. Examples of platforms useful for preparing bispecific antibodies include but are not limited to BiTE (Micromet), DART (MacroGenics) (e.g., U.S. Pat. No. 8,795,667; US Publications 20090060910; 20100174053), Fcab and Mab2 (F-star), Fc-engineered IgG1 (Xencor) or DuoBody (based on Fab arm exchange, Genmab).

In certain embodiments, the multispecific antibodies can include an Fc region. For example, Fc bearing DARTs are heavier, and can bind neonatal Fc receptor, increasing their circulating half-life. See Garber “Bispecific antibodies rise again” Nature Reviews Drug Discovery 13, 799-801 (2014), FIG. 1a; See US Pub 20130295121, incorporated by reference in their entirety.

The invention also provides trispecific antibodies comprising binding specificities of the inventive antibodies. Non-limiting embodiments of trispecific format is described in Xu et al. Science 6 Oct. 2017, Vol. 358, Issue 6359, pp. 85-90; US Pub 20190054182; US Pub 20200054765.

In certain embodiments, the invention encompasses multispecific molecules comprising an Fc domain or portion thereof (e.g. a CH2 domain, or CH3 domain). The Fc domain or portion thereof may be derived from any immunoglobulin isotype or allotype including, but not limited to, IgA, IgD, IgG, IgE and IgM. In some embodiments, the Fc domain (or portion thereof) is derived from IgG. In some embodiments, the IgG isotype is IgG1, IgG2, IgG3 or IgG4 or an allotype thereof.

In some embodiments, the multispecific molecule comprises an Fc domain, which Fc domain comprises a CH2 domain and CH3 domain independently selected from any immunoglobulin isotype (i.e, an Fc domain comprising the CH2 domain derived from IgG and the CH3 domain derived from IgE, or the CH2 domain derived from IgG1 and the CH3 domain derived from IgG2, etc.). In some embodiments, the Fc domain may be engineered into a polypeptide chain comprising the multispecific molecule of the invention in any position relative to other domains or portions of the polypeptide chain (e.g., the Fc domain, or portion thereof, may be c-terminal to both the VL and VH domains of the polypeptide of the chain; may be n-terminal to both the VL and VH domains; or may be N-terminal to one domain and c-terminal to another (i.e., between two domains of the polypeptide chain)).

The present invention also encompasses molecules comprising a hinge domain. The hinge domain be derived from any immunoglobulin isotype or allotype including IgA, IgD, IgG, IgE and IgM. In some embodiments, the hinge domain is derived from IgG, wherein the IgG isotype is IgG1, IgG2, IgG3 or IgG4, or an allotype thereof. The hinge domain may be engineered into a polypeptide chain comprising the multispecific molecule together with an Fc domain such that the multispecific molecule comprises a hinge-Fc domain. In certain embodiments, the hinge and Fc domain are independently selected from any immunoglobulin isotype known in the art or exemplified herein. In other embodiments the hinge and Fc domain are separated by at least one other domain of the polypeptide chain, e.g., the VL domain. The hinge domain, or optionally the hinge-Fc domain, may be engineered into a polypeptide of the invention in any position relative to other domains or portions of the polypeptide chain. In certain embodiments, a polypeptide chain of the invention comprises a hinge domain, which hinge domain is at the C-terminus of the polypeptide chain, wherein the polypeptide chain does not comprise an Fc domain. In yet other embodiments, a polypeptide chain of the invention comprises a hinge-Fc domain, which hinge-Fc domain is at the C-terminus of the polypeptide chain. In further embodiments, a polypeptide chain of the invention comprises a hinge-Fc domain, which hinge-Fc domain is at the N-terminus of the polypeptide chain.

In some embodiments, the invention encompasses multimers of polypeptide chains, each of which polypeptide chains comprise a VH and a VL domain, comprising CDRs as described herein. In certain embodiments, the VL and VH domains comprising each polypeptide chain have the same specificity, and the multimer molecule is bivalent and monospecific. In other embodiments, the VL and VH domains comprising each polypeptide chain have differing specificity and the multimer is bivalent and bispecific. In some embodiments, the polypeptide chains in multimers further comprise an Fc domain. Dimerization of the Fc domains leads to formation of a diabody molecule that exhibits immunoglobulin-like functionality, i.e., Fc mediated function (e.g., Fc-Fc.gamma.R interaction, complement binding, etc.).

In yet other embodiments, diabody molecules of the invention encompass tetramers of polypeptide chains, each of which polypeptide chain comprises a VH and VL domain. In certain embodiments, two polypeptide chains of the tetramer further comprise an Fc domain. The tetramer is therefore comprised of two ‘heavier’ polypeptide chains, each comprising a VL, VH and Fc domain, and two ‘lighter’ polypeptide chains, comprising a VL and VH domain. Interaction of a heavier and lighter chain into a bivalent monomer coupled with dimerization of the monomers via the Fc domains of the heavier chains will lead to formation of a tetravalent immunoglobulin-like molecule. In certain aspects the monomers are the same, and the tetravalent diabody molecule is monospecific or bispecific. In other aspects the monomers are different, and the tetravalent molecule is bispecific or tetraspecific.

Formation of a tetraspecific diabody molecule as described supra requires the interaction of four differing polypeptide chains. Such interactions are difficult to achieve with efficiency within a single cell recombinant production system, due to the many variants of chain mispairings. One solution to increase the probability of mispairings, is to engineer “knobs-into-holes” type mutations into the polypeptide chain pairs. Such mutations favor heterodimerization over homodimerization. For example, with respect to Fc-Fc-interactions, an amino acid substitution (such as a substitution with an amino acid comprising a bulky side group forming a ‘knob’, e.g., tryptophan) can be introduced into the CH2 or CH3 domain such that steric interference will prevent interaction with a similarly mutated domain and will obligate the mutated domain to pair with a domain into which a complementary, or accommodating mutation has been engineered, i.e., ‘the hole’ (e.g., a substitution with glycine). Such sets of mutations can be engineered into any pair of polypeptides comprising the diabody molecule, and further, engineered into any portion of the polypeptides chains of the pair. Methods of protein engineering to favor heterodimerization over homodimerization are well known in the art, in particular with respect to the engineering of immunoglobulin-like molecules, and are encompassed herein (see e.g., Ridgway et al. (1996) “′Knobs-Into-Holes' Engineering Of Antibody CH3 Domains For Heavy Chain Heterodimerization,” Protein Engr. 9:617-621, Atwell et al. (1997) “Stable Heterodimers From Remodeling The Domain Interface Of A Homodimer Using A Phage Display Library,” J. Mol. Biol. 270:26-35, and Xie et al. (2005) “A New Format Of Bispecific Antibody: Highly Efficient Heterodimerization, Expression And Tumor Cell Lysis,” J. Immunol. Methods 296:95-101; each of which is hereby incorporated herein by reference in its entirety).

The invention also encompasses diabody molecules comprising variant Fc or variant hinge-Fc domains (or portion thereof), which variant Fc domain comprises at least one amino acid modification (e.g. substitution, insertion deletion) relative to a comparable wild-type Fc domain or hinge-Fc domain (or portion thereof). Molecules comprising variant Fc domains or hinge-Fc domains (or portion thereof) (e.g., antibodies) normally have altered phenotypes relative to molecules comprising wild-type Fc domains or hinge-Fc domains or portions thereof. The variant phenotype may be expressed as altered serum half-life, altered stability, altered susceptibility to cellular enzymes or altered effector function as assayed in an NK dependent or macrophage dependent assay. Fc domain variants identified as altering effector function are known in the art. For example International Application WO04/063351, U.S. Patent Application Publications 2005/0037000 and 2005/0064514.

The bispecific diabodies of the invention can simultaneously bind two separate and distinct epitopes. In certain embodiments the two separate epitopes are on different cells. In certain embodiments, the two separate epitopes are on two different inhibitory receptors on the same cell. In certain embodiments the epitopes are from the same antigen. In other embodiments, the epitopes are from different antigens. In some embodiments, at least one epitope binding site is specific for a determinant expressed on an immune effector cell (e.g. CD3, CD16, CD32, CD64, etc.) which are expressed on T lymphocytes, natural killer (NK) cells or other mononuclear cells. In one embodiment, the diabody molecule binds to the effector cell determinant and also activates the effector cell. In this regard, diabody molecules of the invention may exhibit Ig-like functionality independent of whether they further comprise an Fc domain (e.g., as assayed in any effector function assay known in the art or exemplified herein (e.g., ADCC assay).

In certain embodiments the bispecific antibodies engage cells for Antibody-Dependent Cell-mediated Cytotoxicity (ADCC). In certain embodiments the bispecific antibodies engage natural killer cells, neutrophil polymorphonuclear leukocytes, monocytes and macrophages. In certain embodiments the bispecific antibodies are T-cell engagers. In certain embodiments, the bispecific antibody comprises a coronavirus binding fragment and a CD3 binding fragment. Various CD3 antibodies are known in the art. See for example U.S. Pat. No. 8,784,821. In certain embodiments, the bispecific antibody comprises a coronavirus binding fragment and CD16 binding fragment.

In certain embodiments the invention provides antibodies with dual targeting specificity. In certain aspects the invention provides bi-specific molecules that can localize an immune effector cell to a coronavirus expressing cell, such as a host cell or a virally infected cell, so as facilitate the killing of this cell. In this regard, bispecific antibodies bind with one “arm” to a surface antigen on target cells, and with the second “arm” to an activating, invariant component of the T cell receptor (TCR) complex or to an activating, invariant component of a different stimulatory receptor such as NKG2C on NK cells or other immune effector cells. The simultaneous binding of such an antibody to both of its targets will force a temporary interaction between target cell and effector cell, causing, for example, activation of any cytotoxic T cell or NK cell and subsequent lysis of the target cell. Hence, the immune response is re-directed to the target cells and may be independent of classical MHC class I peptide antigen presentation by the target cell or the specificity of the T cell as will be relevant for normal MHC-restricted activation of CTLs. In this context it is crucial that CTLs are only activated when a target cell is presenting the bispecific antibody to them, i.e, the immunological synapse is mimicked. For example, embodiments include bispecific antibodies that do not require lymphocyte preconditioning or co-stimulation in order to elicit efficient lysis of target cells.

Several bispecific antibody formats have been developed and their suitability for T cell mediated immunotherapy investigated. Out of these, the so-called BiTE (bispecific T cell engager) molecules have been very well characterized and already shown some promise in the clinic (reviewed in Nagorsen and Bauerle, Exp Cell Res 317, 1255-1260 (2011)). BiTEs are tandem scFv molecules wherein two scFv molecules are fused by a flexible linker. Further bispecific formats being evaluated for T cell engagement include diabodies (Holliger et al., Prot Eng 9, 299-305 (1996)) and derivatives thereof, such as tandem diabodies (Kipriyanov et al., J Mol Biol 293, 41-66 (1999)). DART (dual affinity retargeting) molecules are based on the diabody format that separates cognate variable domains of heavy and light chains of the two antigen binding specificities on two separate polypeptide chains but feature a C-terminal disulfide bridge for additional stabilization (Moore et al., Blood 117, 4542-51 (2011)). Embodiments also comprise Fc-bearing DARTs. The so-called triomabs, which are whole hybrid mouse/rat IgG molecules and also currently being evaluated in clinical trials, represent a larger sized format (reviewed in Seimetz et al., Cancer Treat Rev 36, 458-467 (2010)).

Embodiments comprise bispecific molecules with enhanced pharmacokinetic properties. In some embodiments, such molecules can have increased serum half-life. In some embodiments, these are Fc-bearing DARTs (see supra).

In certain embodiments, such bispecific molecules comprise one portion which targets one portion of the spike protein and a second portion which binds a second target on the spike protein. In certain embodiments, the first portion comprises VH and VL sequences, or CDRs from the antibodies described herein. In certain embodiments, the second target can be, for example but not limited to an effector cell. In certain embodiments the second portion is a T-cell engager. In certain embodiments, the second portion comprises a sequence/paratope which targets CD3, CD16, or another suitable target. In certain embodiments, the second portion is an antigen-binding region derived from a CD3 antibody, optionally a known CD3 antibody. In certain embodiments, the anti-CD antibody induce T cell-mediated or NK-mediated killing. In certain embodiments, the bispecific antibodies are whole antibodies. In other embodiments, the dual targeting antibodies consist essentially of Fab fragments. In other embodiments, the dual targeting antibodies comprise a heavy chain constant region. In certain embodiments, the bispecific antibody does not comprise Fc region. In certain embodiments, the bispecific antibodies have improved effector function. In certain embodiments, the bispecific antibodies have improved cell killing activity. Various methods and platforms for design of bispecific antibodies are known in the art. See for example US Pub. 20140206846, US Pub. 20140170149, US Pub. 20090060910, US Pub 20130295121, US Pub. 20140099318, US Pub. 20140088295 which contents are herein incorporated by reference in their entirety.

### Binding and Neutralization Assays

In some embodiments, the antibodies of the invention are SARS COV-2 neutralizing antibodies. SARS-COV-2 neutralization can be determined by assays known in the art. Non-limiting examples of neutralization assays include PRNT assay, various pseudovirus based neutralization assays are known and described in the art.

Neutralization assays measure neutralizing properties of antibodies. Neutralization properties can correlate with protection from infection and/or therapeutic benefits post infection. In non-limiting examples, neutralization is measured by plaque reduction neutralization test (PRNT). Although PRNT (neutralization) and ELISA (binding) results correlate with each other, PRNT remains the gold-standard for determining neutralization properties.

In other embodiments, fluorescence-based assay that rapidly and reliably measures neutralization of a reporter SARS-COV-2 by antibodies provide a higher throughput neutralization assay.

In some embodiments, neutralization can be determined using a lentivirus-based pseudotyped virus with SARS-COV-2 S protein. With such a system, neutralization can be assessed with respect to the spike protein variants of different SARS COV-2 strains. Strain information and sequence can be obtained from the Global Initiative for Sharing All Influenza Data (GISAID) database and Nextstrain. Non-limiting methods for carrying out neutralization assays are described in the supplementary methods of Korber et al. 2020, Cell182, 812-827.

Nie et al, used the full length S gene from strain Wuhan-Hu-1 with the vesicular stomatitis virus (VSV) pseudovirus system. (Nie et al., Establishment and validation of a pseudovirus neutralization assay for SARS-COV-2, Emerging Microbes Infection, 2020, 9:680-686; citing Whitt, MA., Generation of VSV pseudotypes using recombinant DeltaG-VSV for studies on virus entry, identification of entry inhibitors, and immune responses to vaccines, J Virol Methods, 2010, 169 (2): 365-74.) Briefly, the SARS-COV-2 spike gene can be codon-optimized and cloned into a eukaryotic expression plasmid. 293T cells can be transfected by the plasmid and later infected with a VSV pseudotyped virus (G*DG-VSV), which substitutes the VSV-G gene with luciferase expression cassettes. The culture supernatants are then harvested and filtered 24 h postinfection. The SARS-COV-2 pseudovirus presents the SARS-COV-2 spike protein in the surface of the VSV particle as can be confirmed by Western blot with SARS-COV-2 convalescent patient sera. (Id.)

The SARS-COV-pseudovirus-based neutralization assay (PBNA) tests whether antibodies are able to neutralize SARS-COV-2 pseudovirus infection of susceptible cell-lines (e.g., Vero, Huh7, 293T, HepG2, CHO, MDCK; with Huh7 identified as the best cell substrate for the system), as indicated by inhibition curves of % reduction of RLU relative to antibody sample dilution. (Id.) The SARS-COV-2 pseudovirus can not be neutralized by VSV-G antibodies. In one embodiment, the pseudovirus neutralization assays can be performed using Huh-7 cell lines. Huh-7 are human hepatocellular carcinoma cells that express both ACE2 and TMPRSS2. Various concentrations of mAbs (e.g., 3-fold serial dilution using DMEM, 50 mL aliquots) are mixed with the same volume of SARS-COV-2 pseudovirus with a TCID₅₀ of 1.3×10⁴ in a 96 well-plate. The mixture is incubated for 1 h at 37° C., supplied with 5% CO₂. Negative control wells are supplied with 100 mL DMEM (1% (v/v) antibiotics, 25 nM HEPES, 10% (v/v) FBS). Positive control wells are supplied with 100 mL DMEM. Pre-mixed Huh-7 cells (100 mL, 2 3 105 in DMEM) are added to all wells, and the 96-well plates are incubated for 24 h at 37° C. supplied with 5% CO₂. After the incubation, 150 mL of supernatants are removed, and 100 mL D-luciferin reagent (Invitrogen) is added to each well and incubated for 2 mins. After the incubation, every well is mixed 10 times by pipetting, and 150 mL of the mixture is used to measure luciferase activity using a microplate spectrophotometer (Perkinelmer EnSight). The inhibition rate is calculated by comparing the OD value to the negative and positive control wells. IC50 and IC80 are determined by a four-parameter logistic regression using GraphPad Prism 8.0 (GraphPad Software Inc.).

In some embodiments, neutralization is determined using a SARS COV-2 S pseudotyped virus where the spike protein is the G614 variant. (Korber, B., et al., Tracking changes in SARS-CoV-2 Spike: evidence that D614G increases infectivity of the COVID-19 virus, Cell, Jul. 2, 2020, doi: https://doi.org/10.1016/j.cell.2020.06.043; describing the Spike D614G amino acid change is caused by an A-to-G nucleotide mutation at position 23,403 in the Wuhan reference strain.) The D614G change is almost always accompanied by three other mutations: a C-to-T mutation in the 5′ UTR (position 241relative to the Wuhan reference sequence), a silent C-to-T mutation at position 3,037; and a C-to-T mutation at position 14,408 that results in an amino acid change in RNA-dependent RNA polymerase (RdRp P323L). The haplotype comprising these 4 genetically linked mutations is now the globally dominant form. (Id.) In some embodiments, neutralization is determined using a SARS COV-2 S pseudotyped virus where the spike protein is from strain Wuhan-Hu-1. (GenBank: MN908947.) In some embodiments, a SARS COV-2 S pseudotyped virus uses the VSV pseudovirus or a murine leukemia virus (MLV) pseudotype system. In some embodiments, neutralization assays use authentic SARS-COV-2.

### Production of Antibodies

Antibodies, such as monoclonal antibodies, according to the invention can be made by any method known in the art.

In certain embodiments, plasma cells are cultured in limited numbers, or as single plasma cells in microwell culture plates. Antibodies can be isolated from the plasma cell cultures. VH and VL can be isolated from single cell sorted plasma cells. From the plasma cell, RNA can be extracted and PCR can be performed using methods known in the art. The VH and VL regions of the antibodies can be amplified by RT-PCR (reverse transcriptase PCR), sequenced and cloned into intermediate vectors for further engineering or into an expression vector that is then transfected into HEK293T cells or other host cells as described herein or known in the art. The cloning of nucleic acid in intermediate vectors, expression vectors, the transfection of host cells, the culture of the transfected host cells and the isolation of the produced antibody can be done using any methods known to one of skill in the art. Antibody isolation and purification techniques are known in the art, which can include filtration, centrifugation and various chromatographic methods such as HPLC or affinity chromatography. Techniques for purification of antibodies, e.g., monoclonal antibodies, including techniques for producing pharmaceutical-grade antibodies (and at a sufficiently high concentration or titer for therapeutic use), are well known in the art.

In some embodiments, the antibodies or fragments of the invention have an IgM Fc region or constant domains thereof. It is established that IgM can assume both pentameric and hexameric configurations, depending on the substitution of the J-chain with an additional Fab (2) monomer, which increases the number of Fabs on a single IgM from 10 to 12 (Hiramoto et al Sci. Adv. 2018; 4: eaau1199; Moh E S et al J Am Soc Mass Spectrom. 2016 July;27 (7): 1143-55). Methods for the recombinant production of polymeric IgM (both with and without J chain) have been described (Chromikova et al., Cytotechnology, 2015, 67:343-356; Gilmour et al. Transfusion Medicine, 2008. 18:167-174; Hennicke et al., PLOS ONE, 2020, 15 (3): e0229992). Stable or transient IgM producing cells lines can be generated as described in Chromikova et al., 2015 and Hennicke et al., 2020, where two different pIRES expression vectors are used, one to express the heavy chain and the other to express the light chain and the J-chain sequence. IgM antibodies can be purified according to standard methods in the art, including IgM specific resins for use in affinity chromatography (e.g., POROS Capture Select IgM Affinity Matrix by ThermoFisherScientific.) Transmission electron microscopy (TEM) can be used to confirm pentameric and hexameric forms of IgM.

In addition, besides expression constructs that encode antibody fragments or elements, protein fragments of antibodies can be obtained by methods that include digestion with enzymes, such as pepsin or papain, and/or by cleavage of disulfide bonds by chemical reduction.

Any suitable host cell/vector system may be used for expression of the DNA sequences encoding the antibody molecules of the present invention or fragments thereof. Bacterial, for example E, coli, and other microbial systems may be used, in part, for expression of antibody fragments such as Fab and F(ab′) 2 fragments, and especially Fv fragments and single chain antibody fragments, for example, single chain Fvs, eukaryotic, e.g., mammalian, host cell expression systems may be used for production of larger antibody molecules, including complete antibody molecules. Suitable mammalian host cells include, but are not limited to, CHO, HEK293T, PER.C6, NSO, myeloma or hybridoma cells. Mammalian cell lines suitable for expression of therapeutic antibodies are well known in the art.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides. Alternatively, antibodies according to the invention may be produced by (i) expressing a nucleic acid sequence according to the invention in a host cell, e.g. by use of a vector according to the present invention, and (ii) isolating the expressed antibody product. Additionally, the method may include (iii) purifying the isolated antibody. Transformed B cells and cultured plasma cells can be screened for those producing antibodies of a certain specificity or function.

The screening step may be carried out by any immunoassay, e.g., ELISA, by staining of tissues or cells (including transfected cells), by neutralization assay or by one of a number of other methods known in the art for identifying specificity or function. The assay can select on the basis of simple recognition of one or more antigens, or can select on the additional basis of a function e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

B cell clones or transfected host-cells of the invention can be used in various ways e.g., as a source of monoclonal antibodies, as a source of nucleic acid (DNA or mRNA) encoding a monoclonal antibody of interest, for research, etc.

Expression from recombinant sources is common for pharmaceutical purposes than expression from B cells or hybridomas e.g., for reasons of stability, reproducibility, culture ease, etc.

Thus the invention also provides a method for preparing a recombinant cell, comprising the steps of: (i) obtaining one or more nucleic acids (e.g., heavy and/or light chain mRNAs) from the B cell clone or the cultured plasma cells that encodes the antibody of interest; (ii) inserting the nucleic acid into an expression vector and (iii) transfecting the vector into a host cell in order to permit expression of the antibody of interest in that host cell.

Similarly, the invention provides a method for preparing a recombinant cell, comprising the steps of: (i) sequencing nucleic acid(s) from the B cell clone or the cultured plasma cells that encodes the antibody of interest; and (ii) using the sequence information from step (i) to prepare nucleic acid(s) for insertion into a host cell in order to permit expression of the antibody of interest in that host cell. The nucleic acid may, but need not, be manipulated between steps (i) and (ii) to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest, wherein the nucleic acids are nucleic acids that were derived from a cell sorted B cell or a cultured plasma cell of the invention.

These recombinant cells of the invention can then be used for expression and culture purposes. They are useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

Any suitable host cells can be used for transfection and production of the antibodies of the invention. The transfected host cell may be a eukaryotic cell, including yeast and animal cells, such as mammalian cells (e.g., CHO cells, NSO cells, human cells such as PER.C6 or HKB-11 cells, myeloma cells, or a human liver cell), as well as plant cells. In certain embodiments, expression hosts can glycosylate the antibody of the invention, such as with carbohydrate structures that are not themselves immunogenic in humans. In one embodiment, the transfected host cell may be able to grow in serum-free media. In a further embodiment, the transfected host cell may be able to grow in culture without the presence of animal-derived products. The transfected host cell may also be cultured to give a cell line.

In general, protein therapeutics are produced from mammalian cells. The most widely used host mammalian cells are Chinese hamster ovary (CHO) cells and mouse myeloma cells, including NSO and Sp2/0 cells. Two derivatives of the CHO cell line, CHO-K1 and CHO pro-3, gave rise to the two most commonly used cell lines in large scale production, DUKX-X11 and DG44. (Kim, J., et al., Appl. Microbiol. Biotechnol., 2012, 93:917-30.) Other mammalian cell lines for recombinant antibody expression include, but are not limited to, COS, HeLa, HEK293T, U2OS, A549, HT1080, CAD, P19, NIH 3T3, L929, N2a, HEK 293, MCF-7, Y79, SO-Rb50, HepG2, J558L, and BHK. If the aim is large-scale production, the most currently used cells for this application are CHO cells. Guidelines to cell engineering for mAbs production were also reported. (Costa et al., Eur J Pharm Biopharm, 2010, 74:127-38.) Using heterologous promoters, enhancers and amplifiable genetic markers, the yields of antibody and antibody fragments can be increased. Thus, in certain embodiments, the invention provides an antibody, or antibody fragment, that is recombinantly produced from a mammalian cell-line, including a CHO cell-line. In certain embodiments, the invention provides a composition comprising an antibody, or antibody fragment, wherein the antibody or antibody fragment was recombinantly produced in a mammalian cell-line, and wherein the antibody or antibody fragment is present in the composition at a concentration of at least 1, 10, 100, 1000 micrograms/mL, or at a concentration of at least 1, 2, 3, 4, 5, 6, 7, 8, 9, 10, 20, 50, or 100 milligrams/mL.

Furthermore, large-scale production of therapeutic-grade antibodies are much different than those for laboratory scale. There are extreme purity requirements for therapeutic-grade. Large-scale production of therapeutic-grade antibodies requires multiples steps, including product recovery for cell-culture harvest (removal of cells and cell debris), one or more chromatography steps for antibody purification, and formulation (often by tangential filtration). Because mammalian cell culture and purification steps can introduce antibody variants that are unique to the recombinant production process (i.e., antibody aggregates, N- and C-terminal variants, acidic variants, basic variants, different glycosylation profiles), there are recognized approaches in the art for analyzing and controlling these variants. (See, Fahrner, et al., Industrial purification of pharmaceutical antibodies: Development, operation, and validation of chromatography processes, Biotech. Gen. Eng. Rev., 2001, 18:301-327.) In certain embodiments of the invention, the antibody composition comprises less than 1, 2, 3, 4, 5, 10, 15, 20, 25, 30, 35, 50, or 100 nanograms of host cell protein (i.e., proteins from the cell-line used to recombinantly produce the antibody)). In other embodiments, the antibody composition comprises less than 1, 2, 3, 4, 5, 6, 7, 8, 9, 10, 15, 20, or 25 ng of protein A per milligram of antibody or antibody fragment (i.e., protein A is a standard approach for purifying antibodies from recombinant cell culture, but steps can be done to limit the amount of protein A in the composition, as it may be immunogenic). (See, e.g., U.S. Pat. No. 7,458,704, Reduced protein A leaching during protein A affinity chromatography; which is hereby incorporated-by-reference.)

In certain aspects the invention provides nucleic acids encoding the inventive antibodies. In non-limiting embodiments, the nucleic acids are mRNA, modified or unmodified, suitable for use any use, e.g. but not limited to use as pharmaceutical compositions. In certain embodiments, the nucleic acids are formulated in lipid, such as but not limited to LNPs.

### Pharmaceutical Compositions

The present invention also provides a pharmaceutical composition comprising one or more of: (i) the antibody, or the antibody fragment thereof, according to the present invention; (ii) the nucleic acid encoding the antibody, or antibody fragments according to the present invention; (iii) the vector comprising the nucleic acid according to the present invention; and/or (iv) the cell expressing the antibody according to the present invention or comprising the vector according to the present invention.

In certain aspects, the invention provides a pharmaceutical composition comprising the antibody, or the antigen binding fragment thereof, according to the present invention, the nucleic acid according to the present invention, the vector according to the present invention and/or the cell according to the present invention.

The pharmaceutical composition may also contain a pharmaceutically acceptable carrier, diluent and/or excipient. Although the carrier or excipient may facilitate administration, it can not itself induce the production of antibodies harmful to the individual receiving the composition. Nor can it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In general, pharmaceutically acceptable carriers in a pharmaceutical composition according to the present invention may be active components or inactive components. In certain embodiments the pharmaceutically acceptable carrier in a pharmaceutical composition according to the present invention is not an active component in respect to coronavirus infection.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, capsules, liquids, gels, syrups, slurries and suspensions, for ingestion by the subject.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis. TM, and Herceptin. TM., for reconstitution with sterile water containing a preservative). The composition may be prepared for topical administration e.g., as an ointment, cream or powder. The composition may be prepared for oral administration e.g., as a tablet or capsule, as a spray, or as a syrup (optionally flavored). The composition may be prepared for pulmonary administration e.g., as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a subject. For example, a lyophilized antibody may be provided in kit form with sterile water or a sterile buffer.

A thorough discussion of pharmaceutically acceptable carriers is available in Gennaro (2000) Remington: The Science and Practice of Pharmacy, 20th edition, ISBN: 0683306472.

Pharmaceutical compositions of the invention have a pH between 5.5 and 8.5, in some embodiments this may be between 6 and 8, and in other embodiments about 7. The pH may be maintained by the use of a buffer. The composition may be sterile and/or pyrogen free. The composition may be isotonic with respect to humans. In one embodiment pharmaceutical compositions of the invention are supplied in hermetically-sealed containers.

Within the scope of the invention are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody molecule may be in dry form, for reconstitution before use with an appropriate sterile liquid. A vehicle can be a material that is suitable for storing, transporting, and/or administering a compound, such as a pharmaceutically active compound, for example the antibodies according to the present invention. For example, the vehicle may be a physiologically acceptable liquid, which is suitable for storing, transporting, and/or administering a pharmaceutically active compound, for example the antibodies according to the present invention. Once formulated, the compositions of the invention can be administered directly to the subject. In one embodiment the compositions are adapted for administration to mammalian, e.g., human subjects.

The pharmaceutical compositions of this invention may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays or nebulizers may also be used to administer the pharmaceutical compositions of the invention. In certain embodiments, the pharmaceutical composition may be prepared for oral administration, e.g. as tablets, capsules and the like, for topical administration, or as injectable, e.g. as liquid solutions or suspensions. In certain embodiments, the pharmaceutical composition is an injectable. Embodiments also comprise solid forms suitable for solution in, or suspension in, liquid vehicles prior to injection, e.g. that the pharmaceutical composition is in lyophilized form.

For injection, e.g. intravenous, cutaneous or subcutaneous injection, or injection at the site of affliction, the active ingredient can be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is a polypeptide, peptide, or nucleic acid molecule, other pharmaceutically useful compound according to the present invention that is to be given to an individual, administration is in a “prophylactically effective amount” or a “therapeutically effective amount”, this being sufficient to show benefit to the individual. The actual amount administered, and rate and time-course of administration, will depend on the nature and severity of what is being treated. For injection, the pharmaceutical composition according to the present invention may be provided for example in a pre-filled syringe.

The inventive pharmaceutical composition as defined herein can also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also added. For oral administration in a capsule form, useful diluents include lactose and dried cornstarch. When aqueous suspensions are required for oral use, the active ingredient, i.e, the inventive transporter cargo conjugate molecule as defined herein, is combined with emulsifying and suspending agents. In embodiments, certain sweetening, flavoring or coloring agents can also be added.

The inventive pharmaceutical composition can also be administered topically, especially when the target of treatment includes areas or organs readily accessible by topical application, e.g. including diseases of the skin or of any other accessible epithelial tissue. Suitable topical formulations are readily prepared for each of these areas or organs. For topical applications, the inventive pharmaceutical composition can be formulated in a suitable ointment, containing the inventive pharmaceutical composition, for example its components as defined herein, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the inventive pharmaceutical composition can be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

Suitable dose ranges can depend on the antibody (or fragment) and on the nature of the formulation and route of administration. For example, doses of antibodies in the range of 0.1-50 mg/kg, 1-50 mg/kg, 1-10 mg/kg, 1, 1.25, 1.5, 2, 2.5, 3, 3.5, 4, 4.5, 5, 5.5, 6, 6.5, 7, 7.5, 8, 8.5, 9, 9.5 or 10 mg/kg of antibody can be used. If antibody fragments are administered, then less antibody can be used (e.g., from 5 mg/kg to 0.01 mg/kg). In other embodiments, the antibodies of the invention can be administered at a suitable fixed dose, regardless of body size or weight. See Bai et al. Clinical Pharmacokinetics February 2012, Volume 51, Issue 2, pp 119-135.

Dosage also depends on whether the composition is administered as a recombinant protein or a nucleic acid.

Dosage treatment can be a single dose schedule or a multiple dose schedule. For example, the pharmaceutical composition can be provided as single-dose product. In certain embodiments, the amount of the antibody in the pharmaceutical composition—for example if provided as single-dose product--does not exceed 200 mg. In certain embodiments, the amount does not exceed 100 mg, and in certain embodiments, the amount does not exceed 50 mg.

In non-limiting embodiments, the antibodies of the invention can be used for non-therapeutic uses, such as but not limited to diagnostic assays. In non-limiting embodiments, the antibodies can be used for serology testing in any suitable assay or format, including without limitation sandwich ELISA based detection.

### Administration of Antibody Encoding Nucleic Acid Sequences

In some embodiments the antibodies are administered as nucleic acids, including but not limited to mRNAs which can be modified and/or unmodified. See US Pub 20180028645A1,, US Pub 20090286852, US Pub 20130111615, US Pub 20130197068, US Pub 20130261172, US Pub 20150038558, US Pub 20160032316, US Pub 20170043037, US Pub 20170327842, U.S. Pat. Nos. 10,006,007, 9,371,511, 9,012,219, US Pub 20180265848, US Pub

20170327842, US Pub 20180344838A1 at least at paragraphs-[0281], WO/2017/182524 for non-limiting embodiments of chemical modifications, wherein each content is incorporated by reference in its entirety.

mRNAs delivered in LNP formulations have advantages over non-LNPs formulations. See US Pub 20180028645A1, WO/2018/081638, WO/2016/176330, wherein each content is incorporated by reference in its entirety.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding the polypeptide sequence of the sequences in the application, or a variant thereof or a fragment thereof. Accordingly, in one embodiment, the invention provides an RNA molecule encoding one or more of inventive antibodies. The RNA may be plus-stranded. Accordingly, in some embodiments, the RNA molecule can be translated by cells without needing any intervening replication steps such as reverse transcription.

In some embodiments, a RNA molecule of the invention may have a 5′ cap (e.g. but not limited to a 7-methylguanosine, 7 mG (5′) ppp (5′) NlmpNp). This cap can enhance in vivo translation of the RNA. The 5′ nucleotide of an RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. In some embodiments, a RNA molecule useful with the invention may be single-stranded. In some embodiments, a RNA molecule useful with the invention may comprise synthetic RNA.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

In certain aspects, the invention provides an in vitro transcription system to synthesize ribonucleic acids (RNAs) encoding antibodies of the invention, comprising: a reaction vessel, a DNA vector template comprising nucleic acid sequence encoding an antibody of the invention as described in Tables 3-6, 10 or 11, and reagents for carrying out an in vitro transcription reaction that produces mRNA encoding an antibody or fragment thereof of the invention. In certain embodiments the mRNA is modified mRNA.

In certain aspects, the invention provides an in vitro transcription system to synthesize ribonucleic acids (RNAs) encoding antibodies of the invention, comprising: a reaction vessel, a DNA vector template comprising nucleic acid sequence encoding an antibody of the invention as described in Tables 3-6, 10 or 11, and reagents for carrying out an in vitro transcription reaction that produces mRNA encoding an antibody or fragment thereof of the invention. Methods to purify mRNA and assay its purity for therapeutic use are known in the art. In certain embodiments the mRNA is modified mRNA. A skilled artisan can readily scale up the in vitro transcription reaction methods, purification and analytical methods for large scale mRNA batches.

### Therapeutic Methods

In certain aspects, the invention provides prophylactic and/or therapeutic methods comprising administering the antibodies of the invention in an amount suitable to effect prophylaxis or treatment. In certain embodiments, the methods of administering antibodies lead to protection from acquiring of infection, or reducing severity of infection or disease by binding coronavirus spike protein and neutralizing coronavirus. Therapeutic doses depend on the mode of delivery and whether the antibody is delivered as a recombinant protein or a nucleic acid.

### Diagnostic Methods

In certain aspects the invention provides methods for detecting coronavirus virus in a sample suspected of containing said coronavirus virus, comprising (1) contacting the sample with a first antibody or antigen-binding fragment thereof binding coronavirus, and assaying binding of the antibody with said sample for formation of first antibody-coronavirus sample, wherein in some embodiments the first antibody is immobilized on a suitable surface or the first antibody-coronavirus complex is immobilized, (2) removing unbound sample and/or first coronavirus antibody, (3) contacting the immobilized first antibody-coronavirus sample complex with a second antibody, and assaying binding of the second coronavirus antibody to the first antibody-coronavirus complex, wherein the second antibody has a different binding epitope from the first antibody, and wherein the first and/or the second antibody is any one of the antibodies of the invention, e.g. Table 3-6, 10 or 11. Techniques to assay and quantitate binding between antibody and target epitope in a sample are known in the art. In certain embodiments, the second antibody is conjugated for direct detection. In non-limiting embodiments, the antibody-coronavirus complex is indirectly detected by a detection reagent which binds the second coronavirus antibody. In non-limiting embodiments, the detection reagent can be another antibody conjugated to comprises any suitable imaging agent including without limitation color detection, a fluorophore, a magnetic nano-particle, or a radionuclide. Non-limiting examples include any variation of ELISA sandwich-based immunoassay.

In certain embodiments, the sample is any suitable sample including but not limited to respiratory tract secretions, saliva, nasal swabs, etc.

In certain aspects, the invention provides kits comprising the inventive antibodies, reagents and instructions for therapeutic or diagnostic use.

CDRs can be identified by any method known in the art. In some embodiment, CDRs as identified are identified using IMGT.

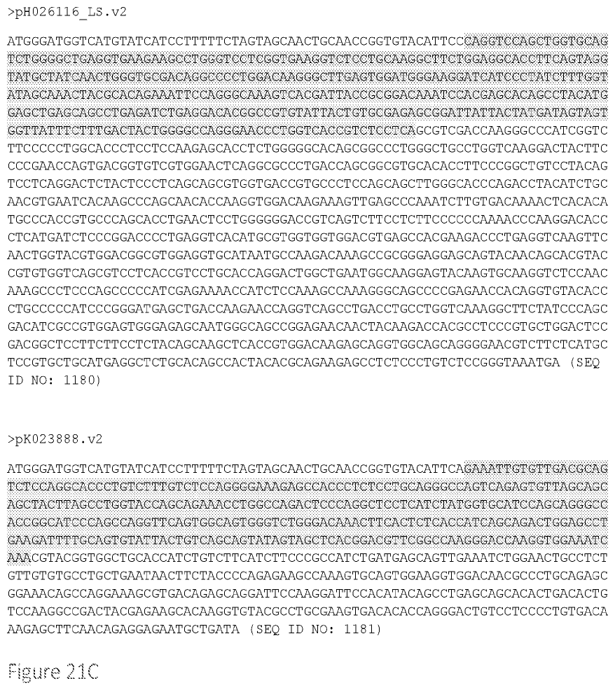

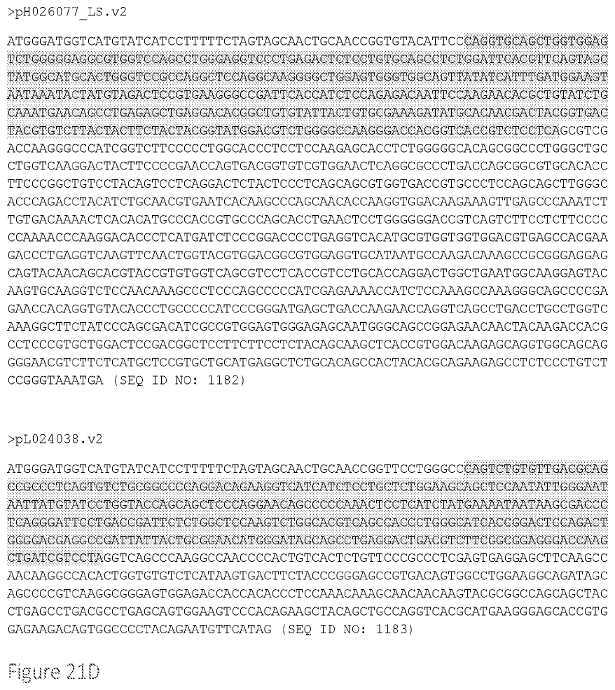

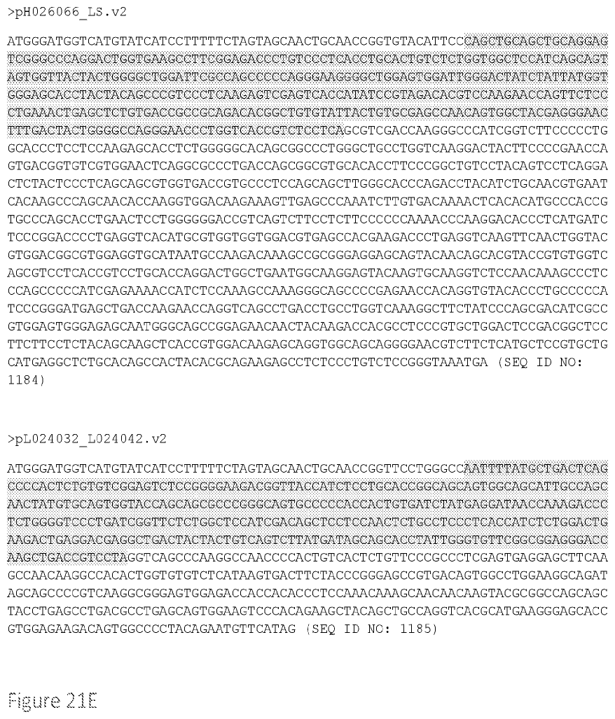

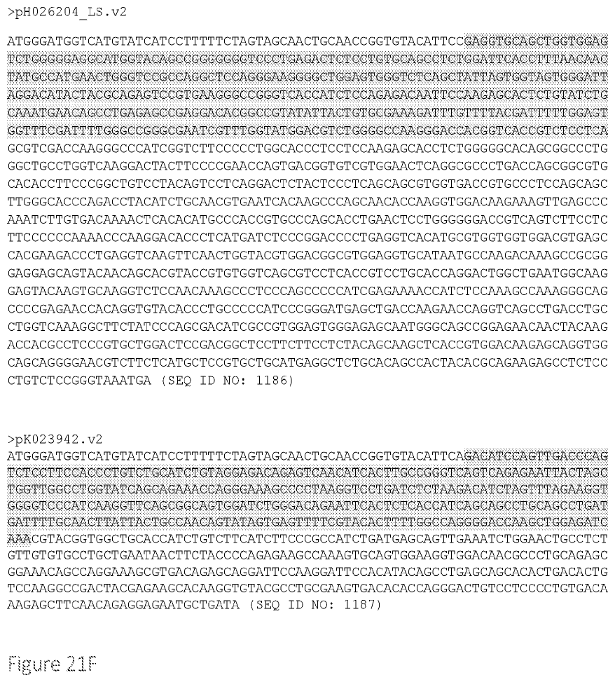

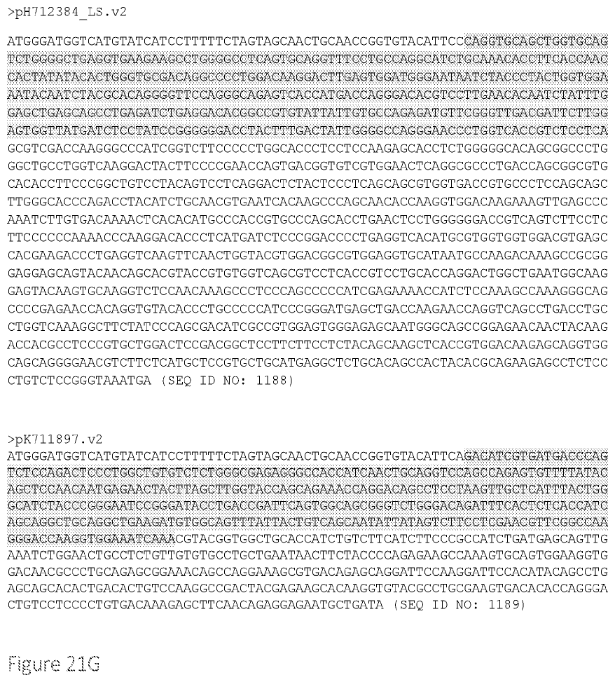

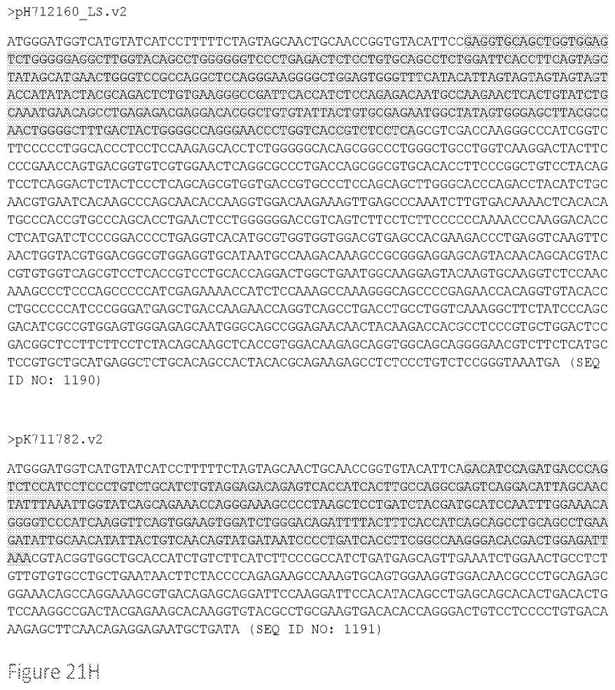

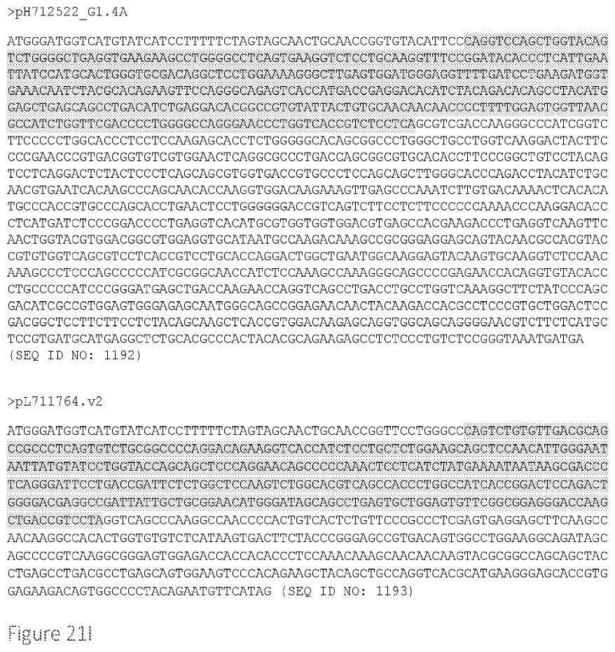

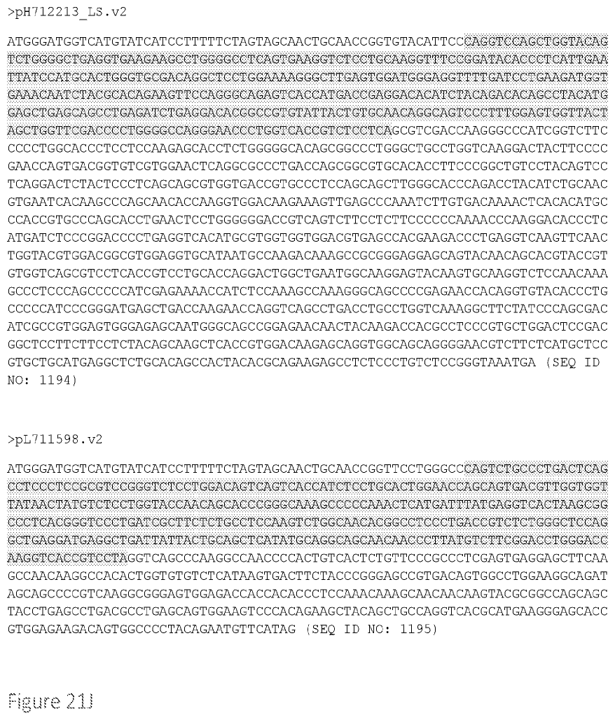

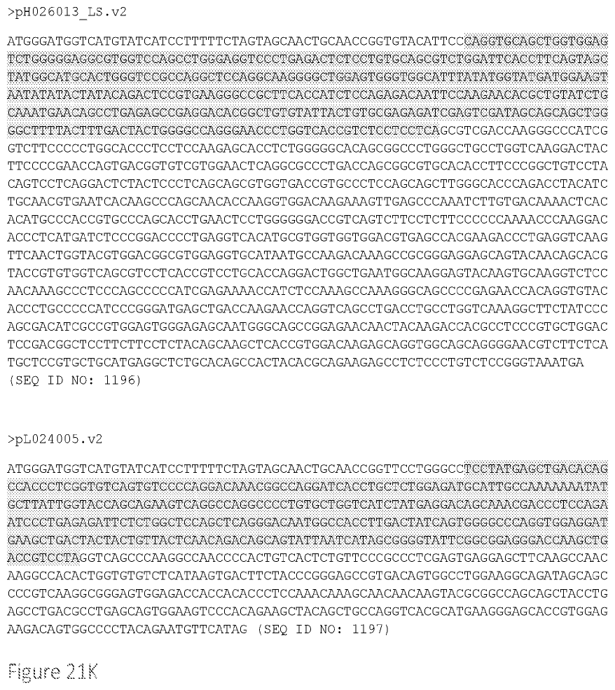

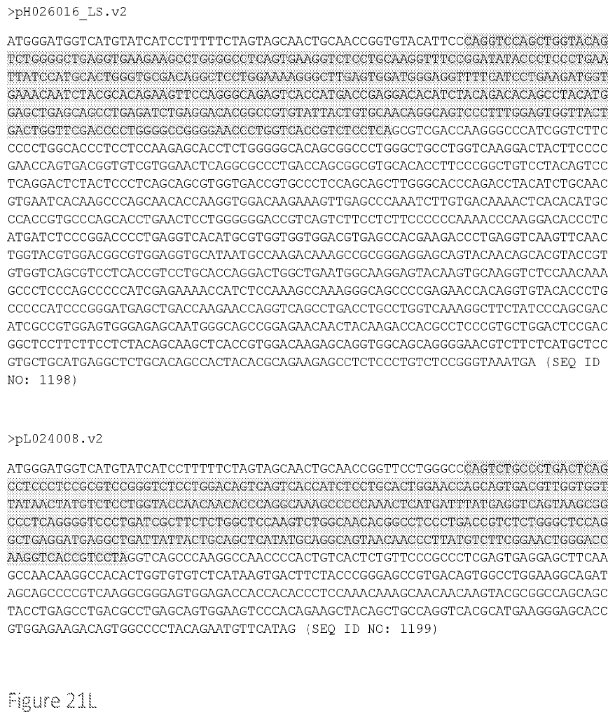

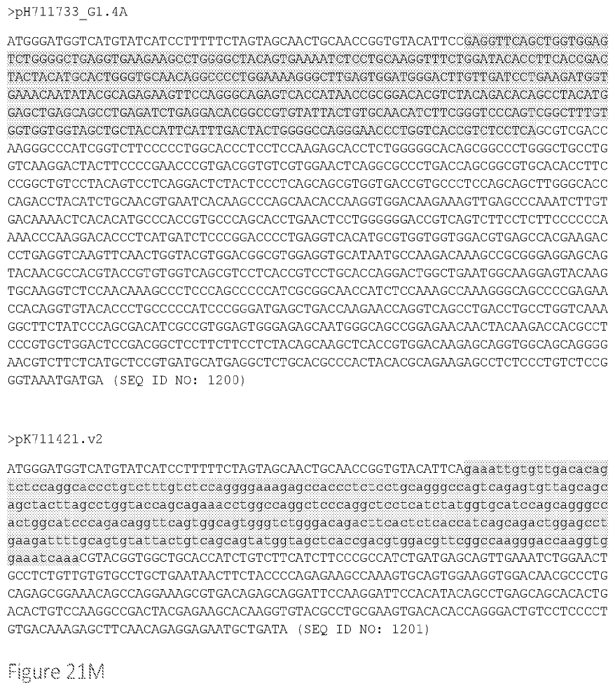

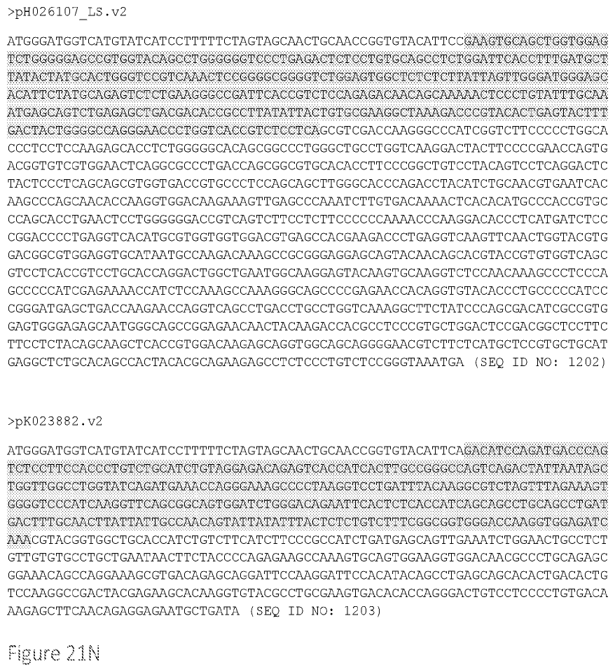

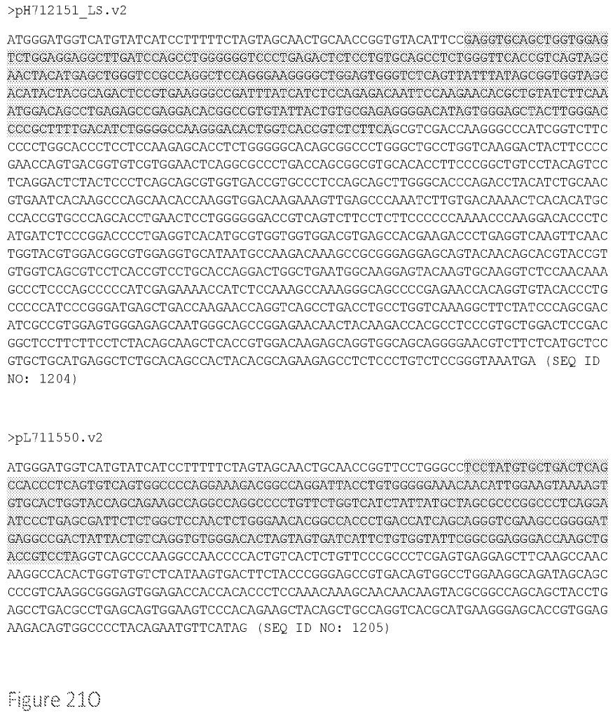

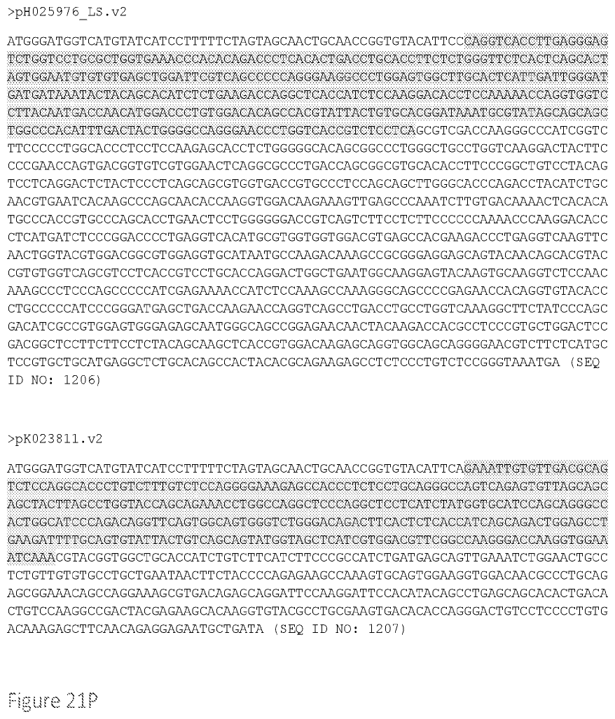

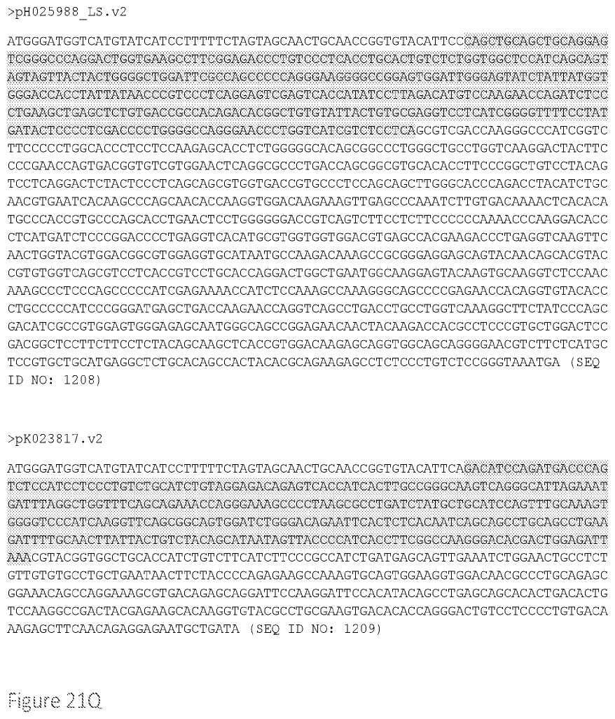

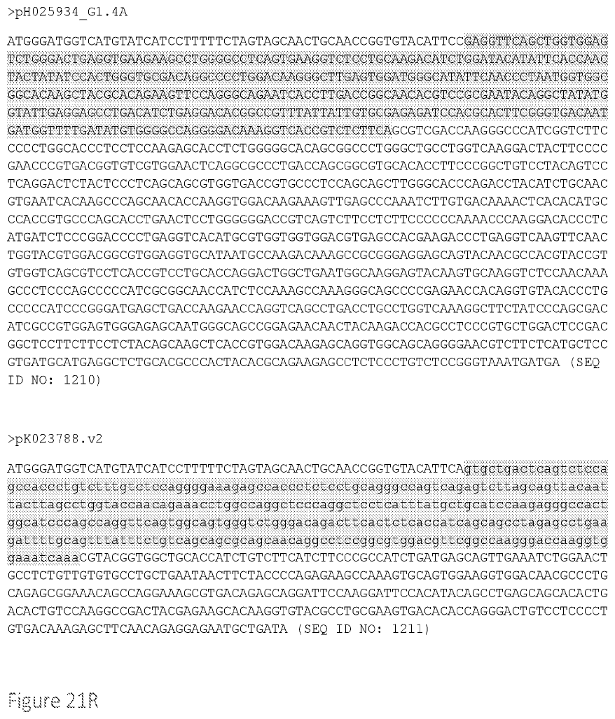

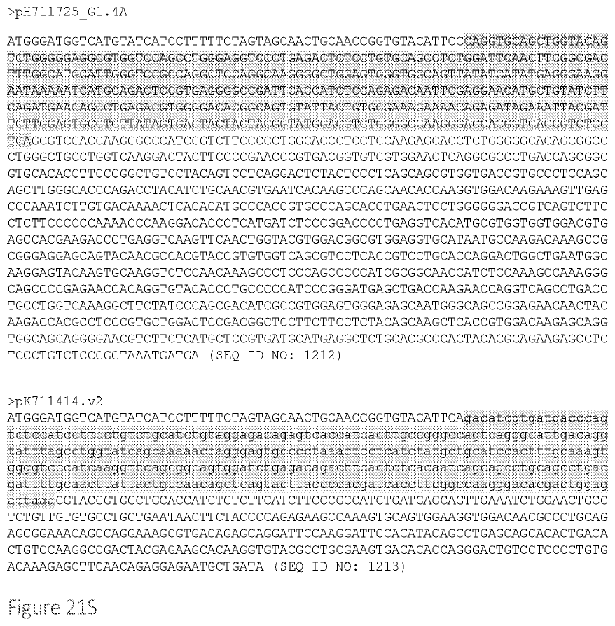

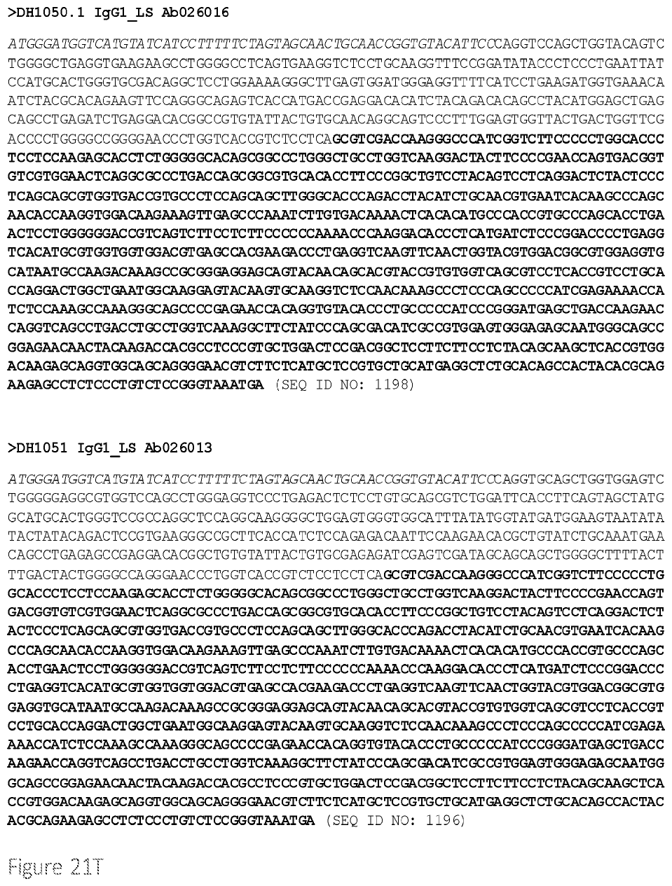

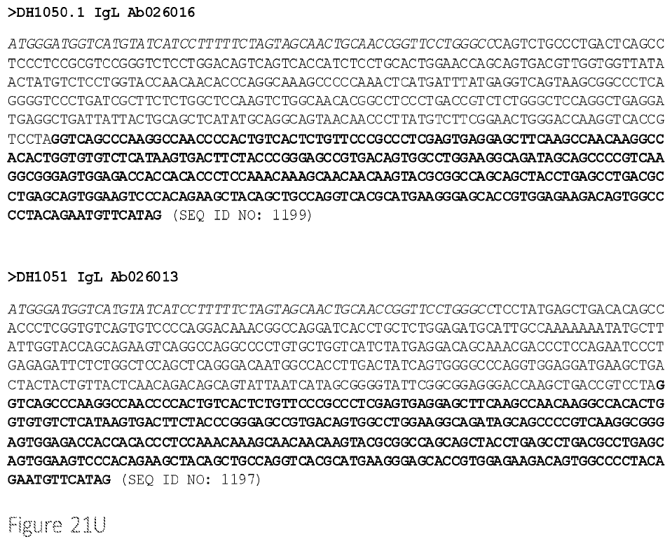

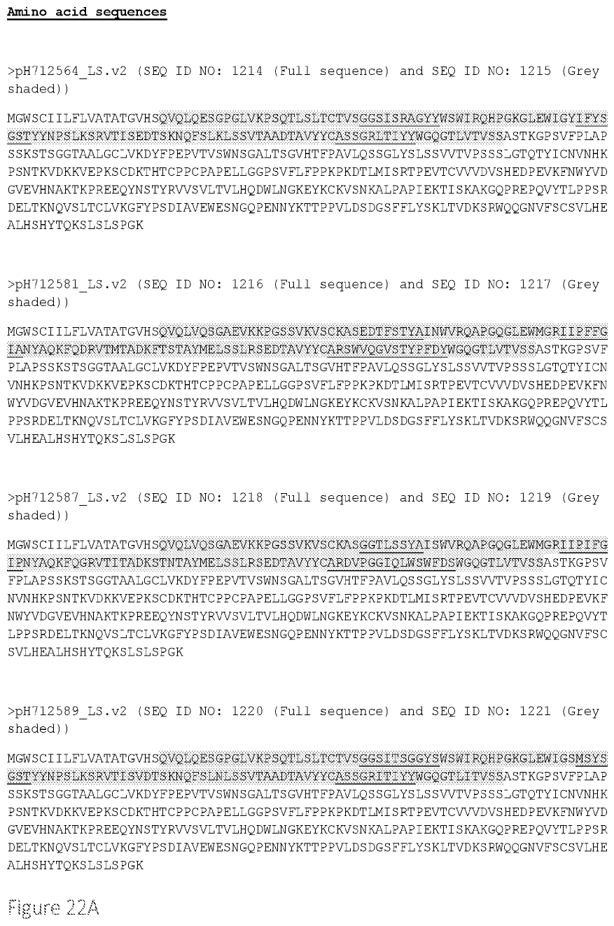

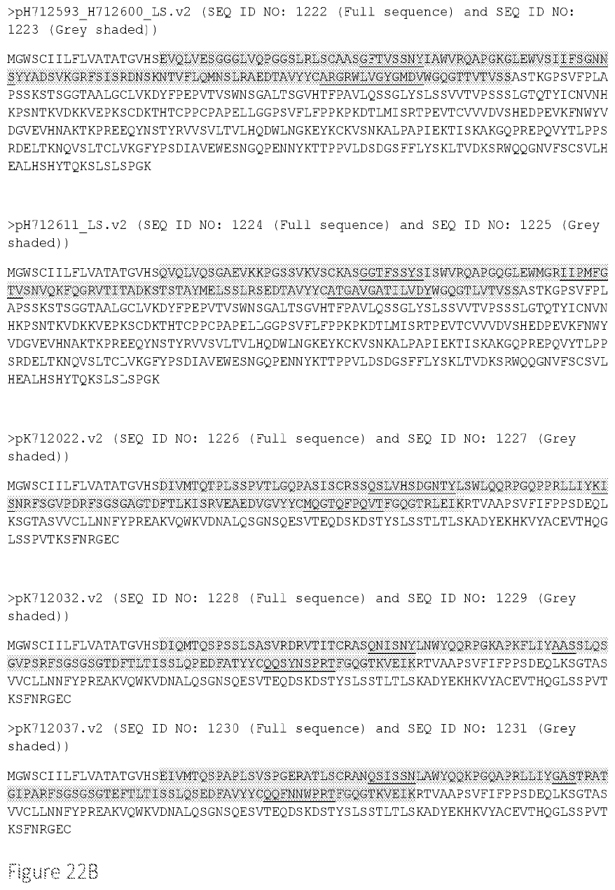

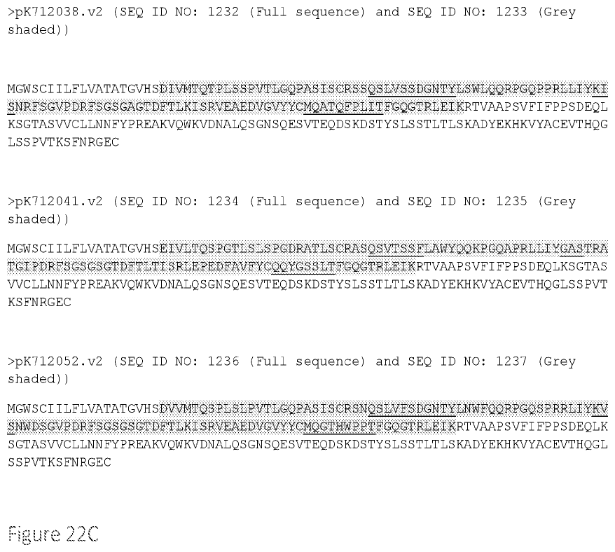

TABLE 3
Summary of antibodies (includes DH nomenclature, VH and VL IDs and gene information,
specificity and clonal relationship). See also FIGS. 20-21 for sequences.

| CH/HG/DH | | | | | | | | Clonal |
|---|---|---|---|---|---|---|---|---|
| # | PTID | Ab ID | VH ID | VL ID | VH gene | VL gene | Notes | Relationship(s) |
| DH1041 | 001-ED- | Ab026124_LS | pH026124_LS.v2 | pL024055.v2 | IGHV3- | IGLV1- | RBD-specific, | |
| | 026 | | | | 7*02 | 40*01 | ACE2-blocking | |
| | | | | | | | Nab | |
| DH1042 | 001-ED- | Ab026103_LS | pH026103_LS.v2 | pK023879.v2 | IGHV1- | IGKV1- | RBD-specific, | |
| | 026 | | | | 69*04 | 39*01 | ACE2-blocking | |
| | | | | | | | Nab | |
| DH1043 | 001-ED- | Ab026116_LS | pH026116_LS.v2 | pK023888.v2 | IGHV1- | IGKV3- | RBD-specific, | |
| | 026 | | | | 69*04 | 20*01 | ACE2-blocking | |
| | | | | | | | Nab | |
| DH1044 | 001-ED- | Ab026077_LS | pH026077_LS.v2 | pL024038.v2 | IGHV3-30- | IGLV1- | RBD-specific, | |
| | 026 | | | | 5*01 | 51*02 | non-ACE2- | |
| | | | | | | | blocking Nab | |
| DH1045 | 001-ED- | Ab026066_LS | pH026066_LS.v2 | pL024032_{—} | IGHV4- | IGLV6- | RBD-specific, | |
| | 026 | | | L024042.v2 | 39*01 | 57*02 | Cross-reactive | |
| DH1046 | VRC200 | Ab026204_LS | pH026204_LS.v2 | pK023942.v2 | IGHV3- | IGKV1- | RBD-specific, | |
| | | | | | 23D*02 | 5*03 | Cross-reactive | |
| DH1047 | VRC200 | Ab712384_LS | pH712384_LS.v2 | pK711897.v2 | IGHV1- | IGKV4- | RBD-specific, | |
| | | | | | 46*03 | 1*01 | Cross-reactive | |
| DH1048 | 001-ED- | Ab712160_LS | pH712160_LS.v2 | pK711782.v2 | IGHV3- | IGKV1- | NTD Nab | |
| | 026 | | | | 48*02 | 33*01 | | |
| DH1049 | 001-ED- | Ab712522_G1.4A | pH712522_LS.v2 | pL711764.v2 | IGHV1- | IGLV1- | NTD Nab | |
| | 029 | | | | 24*01 | 51*02 | | |
| DH1050.1 | 001-ED- | Ab026016_LS | pH026016_LS.v2 | pL024008.v2 | IGHV1- | IGLV2- | NTD Nab | Clonal |
| | 026 | | | | 24*01 | 8*01 | | lineage |
| DH1050.2 | 001-ED- | Ab712213_LS | pH712213_LS.v2 | pL711598.v2 | IGHV1- | IGLV2- | NTD Nab | |
| | 026 | | | | 24*01 | 8*01 | | |
| DH1051 | 001-ED- | Ab026013_LS | pH026013_LS.v2 | pL024005.v2 | IGHV3- | IGLV3- | NTD Nab | |
| | 026 | | | | 33*01 | 10*01 | | |
| DH1052 | 001-ED- | Ab711733_G1.4A | pH711733_G1.4A | pK711421.v2 | IGHV1-69- | IGKV3- | NTD ADE | |
| | 026 | | | | 2*01 | 20*01 | | |
| DH1053 | 001-ED- | Ab026107_LS | pH026107_LS.v2 | pK023882.v2 | IGHV3- | IGKV1- | NTD ADE | |
| | 026 | | | | 43*01 | 5*03 | | |
| DH1054 | 001-ED- | Ab712151_LS | pH712151_LS.v2 | pL711550.v2 | IGHV3- | IGLV3- | NTD ADE | |
| | 026 | | | | 53*01 | 21*01 | | |
| DH1055 | 001-ED- | Ab025976_LS | pH025976_LS.v2 | pK023811.v2 | IGHV2- | IGKV3- | NTD ADE | |
| | 026 | | | | 70*01 | 20*01 | | |
| DH1056 | 001-ED- | Ab025988_LS | pH025988_LS.v2 | pK023817.v2 | IGHV4- | IGKV1- | NTD ADE | |
| | 026 | | | | 39*01 | 17*01 | | |
| DH1057 | 001-ED- | Ab025934_G1.4A | pH025934_G1.4A | pK023788.v2 | IGHV1- | IGKV3- | S2 Nab | |
| | 026 | | | | 46*01 | 11*01 | | |
| DH1058 | 001-ED- | Ab711725_G1.4A | pH711725_G1.4A | pK711414.v2 | IGHV3-30- | IGKV1- | S2 non-Nab, | |
| | 026 | | | | 5*01 | 9*01 | broadly cross- | |
| | | | | | | | reactive | |

TABLE 4A
Summary of antibodies (including DH names, specificity groups and blocking,
cross-reactivity, IC50, etc.). See FIGS. 20-21 for sequences.

| | | | | | Blocking | |
|---|---|---|---|---|---|---|
| Binding | | DH | | | IC50 | IC80 |
| Domain | Group | number | Antibody ID | Cross Reactivity | (μg/ml) | (μg/ml) |
| RBD | ACE2-blocking | DH1041 | Ab026124_LS | No | <0.2 | 0.497 |
| | Nab | DH1042 | Ab026103_LS | No | <0.2 | 0.371 |
| | | DH1043 | Ab026116_LS | No | <0.2 | 0.28 |
| | non-ACE2- | DH1044 | Ab026077_LS | No | >50 | >50 |
| | blocking Nab | | | | | |
| | Cross-reactive | DH1045 | Ab026066_LS | SARS-CoV-1 | 0.226 | 24.353 |
| | | DH1046 | Ab026204_LS | SARS-CoV-1 | 0.201 | 6.512 |
| | | DH1047 | Ab712384_LS | SARS-CoV-1 | 0.078 | 0.567 |
| NTD | NTD Nab | DH1048 | Ab712160_LS | No | >50 | >50 |
| | | DH1049 | Ab712522_G1.4A | No | >50 | >50 |
| | | DH1050.2 | Ab712213_LS | No | >50 | >50 |
| | | DH1051 | Ab026013_LS | No | >50 | >50 |
| | | DH1050.1 | Ab026016_LS | No | >50 | >50 |
| | NTD; non- | DH1052 | Ab711733_G1.4A | No | >50 | >50 |
| | neutralizing | | | | | |
| | NTD; Non- | DH1053 | Ab026107_LS | No | >50 | >50 |
| | neutralizing | | | | | |
| | NTD; Non- | DH1054 | Ab712151_LS | No | >50 | >50 |
| | neutralizing | | | | | |
| | NTD; Non- | DH1055 | Ab025976_LS | No | >50 | >50 |
| | neutralizing | | | | | |
| | NTD; Non- | DH1056 | Ab025988_LS | No | >50 | >50 |
| | neutralizing | | | | | |
| S2 | S2 Nab | DH1057 | Ab025934_G1.4A | SARS-CoV-1, | >50 | >50 |
| | | | | OC43 | | |
| | S2 non-Nab, | DH1058 | Ab711725_G1.4A | SARS-CoV-1, | >50 | >50 |
| | broadly cross- | | and clonal lineage | MERS-CoV, 229E, | | |
| | reactive | | members | NL63, HKU1, | | |
| | | | | OC43 | | |

| | | | | SARS-CoV-2 neutralization | | | |
|---|---|---|---|---|---|---|---|
| | | | | Minimal | | | |
| | Pseudovirus neutralization | | | Effective | | | |
| Binding | IC50 | IC80 | | conc. | IC50 | IC80 | IC90 |
| Domain | (μg/ml) | (μg/ml) | MPI | (μg/ml) | (μg/ml) | (μg/ml) | (μg/ml) |
| RBD | 0.017 | 0.049 | 100 | <0.098 | 0.016 | 0.063 | 0.143 |
| | 0.011 | 0.053 | 100 | 0.28 | 0.071 | 0.269 | 0.578 |
| | 0.0015 | 0.02 | 100 | <0.098 | 0.034 | 0.099 | 0.169 |
| | 0.021 | 0.08 | 98 | 0.55 | 0.076 | 0.273 | 0.577 |
| | 0.38 | 2.26 | 100 | 6.25 | 1.437 | 4.827 | 9.806 |
| | 0.396 | 2.03 | 100 | 12.5 | 1.086 | 9.383 | 17.55 |
| | 0.09 | 0.36 | 100 | 1.1 | 0.1244 | 0.6656 | 1.775 |
| NTD | 0.52 | >50 | 72 | 0.39 | 0.608 | 2.232 | 4.775 |
| | >50 | >50 | 49 | 0.39 | 0.385 | 3.539 | 12.89 |
| | 0.28 | >50 | 67 | 0.78 | 0.087 | 1.187 | 5.47 |
| | 0.049 | >50 | 68 | 0.78 | 0.134 | 0.737 | 1.995 |
| | 0.039 | >50 | 62 | 0.78 | 0.161 | 0.614 | 1.341 |
| | >50 | >50 | −148 | >100 | ND | ND | ND |
| | >50 | >50 | −99 | >100 | ND | ND | ND |
| | >50 | >50 | −63 | >100 | ND | ND | ND |
| | >50 | >50 | −106 | >100 | ND | ND | ND |
| | >50 | >50 | −56 | >100 | ND | ND | ND |
| S2 | >50 | >50 | 46 | 17.68 | 10.89 | 44.1 | 99.93 |
| | >43.5 | >43.5 | −13 | >100 | ND | ND | ND |

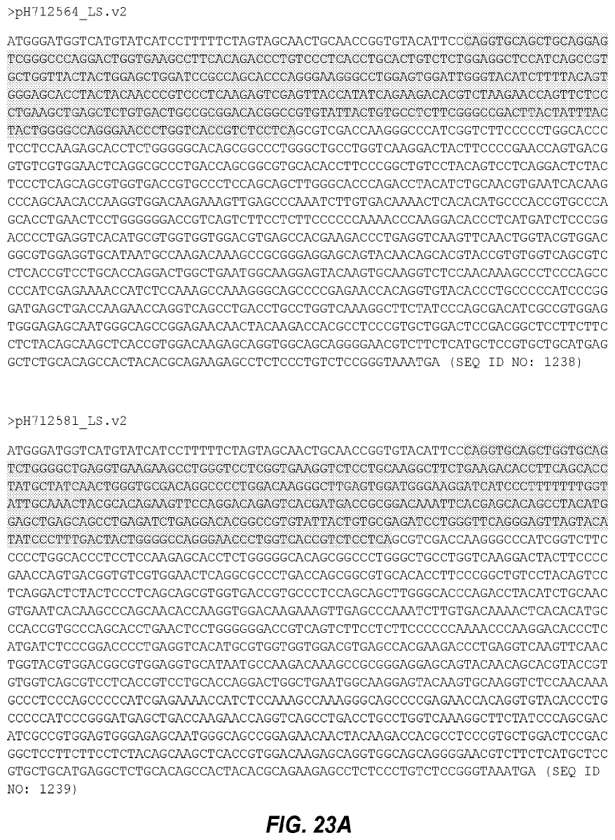

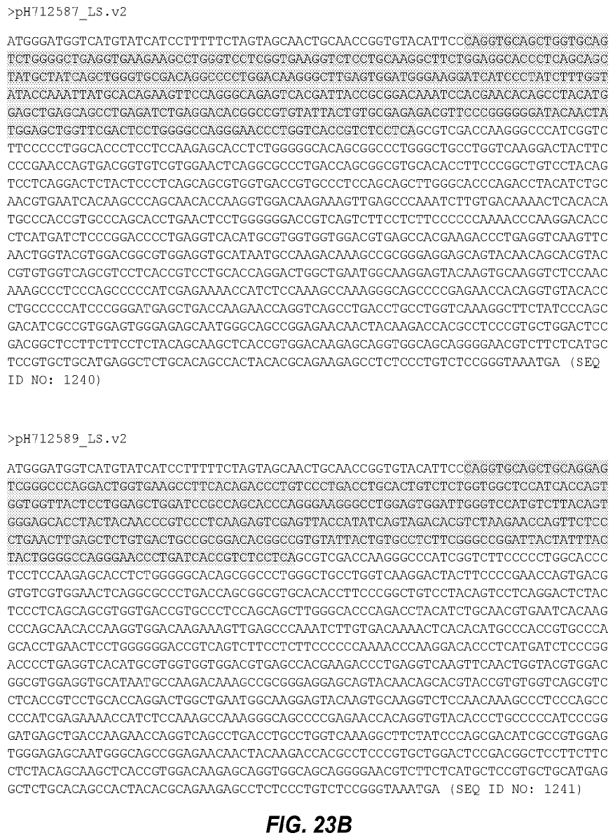

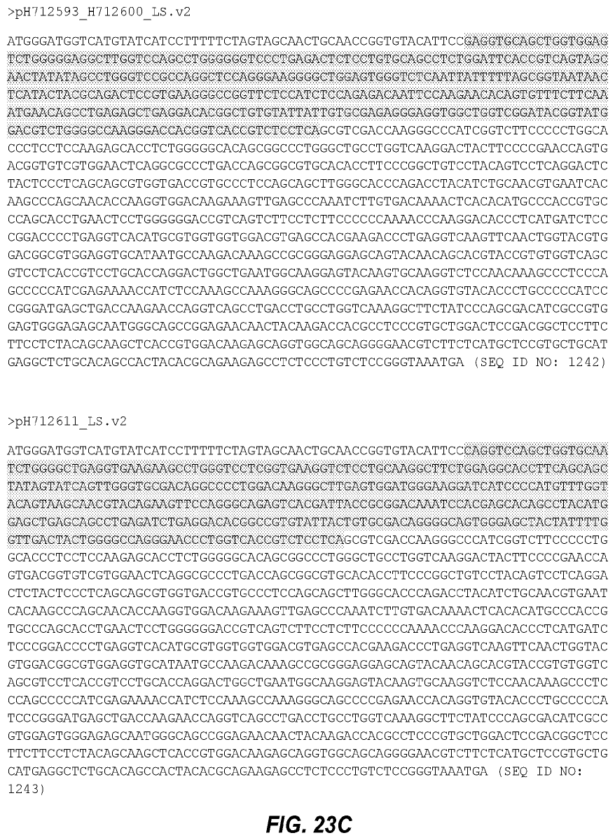

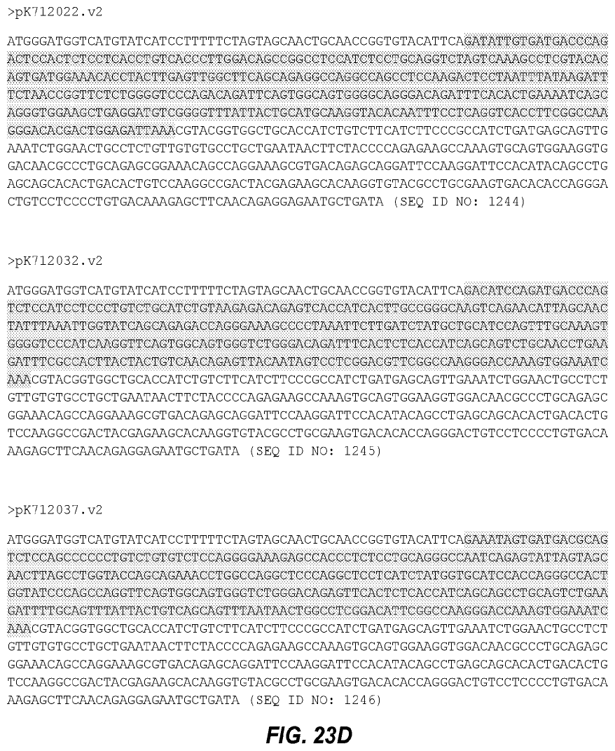

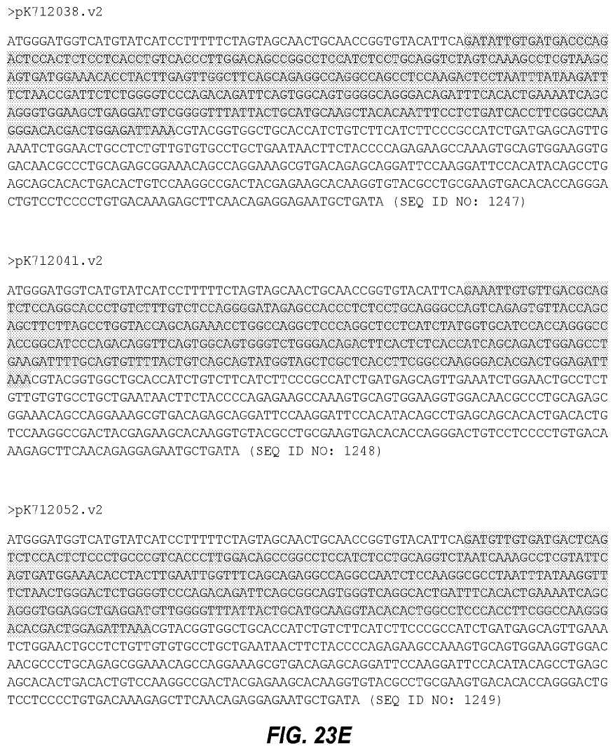

TABLE 4B
Summary of antibodies (including DH names). See FIGS. 22 and 23 for sequences.

| | | | | | | Neutralization | | | | | |
|---|---|---|---|---|---|---|---|---|---|---|---|
| | | | | | | | | | SARS-CoV-2 virus | | |
| | | | | ACE-2 Blocking | | Pseudovirus | | | Minimal | | |
| | | Binding | Cross | IC50 | IC80 | IC50 | IC80 | | Effective | IC50 | IC80 |
| DH# | Antibody ID | Specificity | Reactivity | (μg/ml) | (μg/ml) | (μg/ml) | (μg/ml) | MPI | conc. (μg/ml) | (μg/ml) | (μg/ml) |
| DH1221 | Ab712564_LS | RBD | No | 0.0343 | 0.06815 | 0.004 | 0.015 | 100 | <0.0976 | 0.007167 | 0.04929 |
| DH1222 | Ab712581_LS | RBD | No | 0.03664 | 0.06599 | 0.010 | 0.031 | 100 | <0.0976 | 0.009418 | 0.01559 |
| DH1223 | Ab712587_LS | RBD | No | 0.02763 | 0.06656 | 0.003 | 0.031 | 100 | <0.0976 | 0.005281 | 0.08982 |
| DH1224 | Ab712589_LS | RBD | No | 0.03083 | 0.06511 | 0.009 | 0.032 | 100 | <0.0976 | 0.009225 | 0.02679 |
| DH1225 | Ab712593_{—} | RBD | No | 0.03705 | 0.07397 | 0.006 | 0.015 | 100 | <0.0976 | 0.008233 | 0.02512 |
| | 712600K_LS | | | | | | | | | | |
| DH1226 | Ab712611_LS | RBD | No | 0.04159 | 0.105 | 0.008 | 0.032 | 100 | <0.0976 | 0.009656 | 0.04587 |

TABLE 4C
Gene usage of antibodies from Table 4B

| CH/HG/DH# | PTID | Ab ID | VH ID | VL ID | VH gene | VL gene | Notes |
|---|---|---|---|---|---|---|---|
| DH1221 | 001-ED- | Ab712564_LS/293i/Citrate | H712564 | K712022 | IGHV4- | IGKV2- | RBD-specific, |
| | 26 | | | | 31 | 24 | ACE2-blocking Nab |
| DH1222 | 001-ED- | Ab712581_LS/293i/Citrate | H712581 | K712032 | IGHV1- | IGKV1- | RBD-specific, |
| | 26 | | | | 69 | 39 | ACE2-blocking Nab |
| DH1223 | 001-ED- | Ab712587_LS/293i/Citrate | H712587 | K712037 | IGHV1- | IGKV3- | RBD-specific, |
| | 26 | | | | 69 | 15 | ACE2-blocking Nab |
| DH1224 | 001-ED- | Ab712589_LS/293i/Citrate | H712589 | K712038 | IGHV4- | IGKV2- | RBD-specific, |
| | 26 | | | | 31 | 24 | ACE2-blocking Nab |
| DH1225 | 001-ED- | Ab712593_712600K_LS/293i/Citrate | H712593 | K712041 | IGHV3- | IGKV3- | RBD-specific, |
| | 26 | | | | 66 | 20 | ACE2-blocking Nab |
| DH1226 | 001-ED- | Ab712611_LS/293i/Citrate | H712611 | K712052 | IGHV1- | IGKV2D- | RBD-specific, |
| | 26 | | | | 69 | 30 | ACE2-blocking Nab |

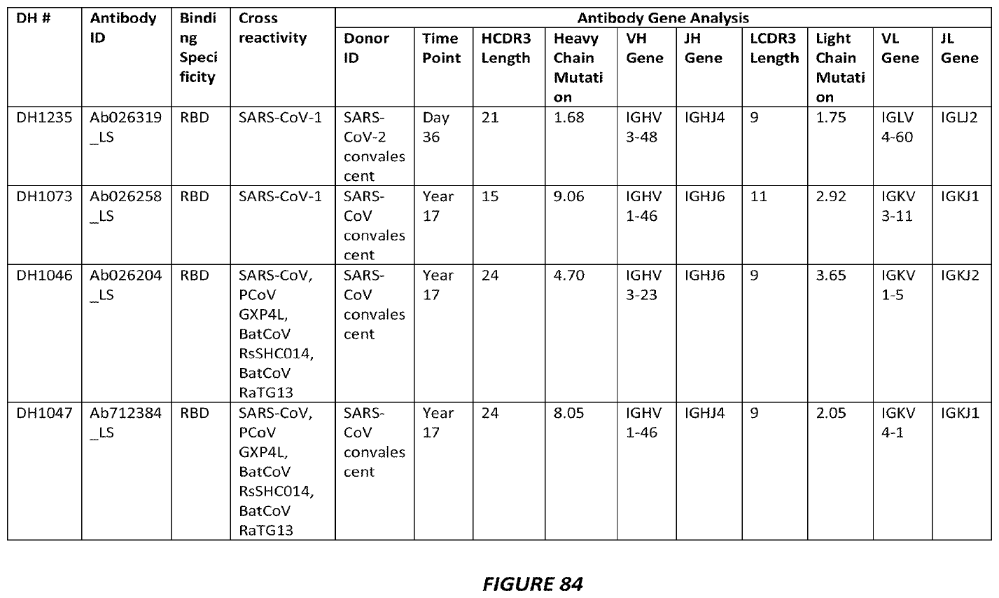

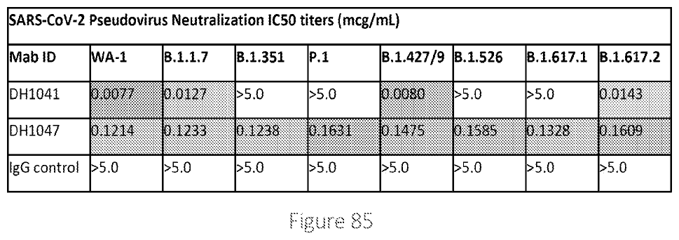

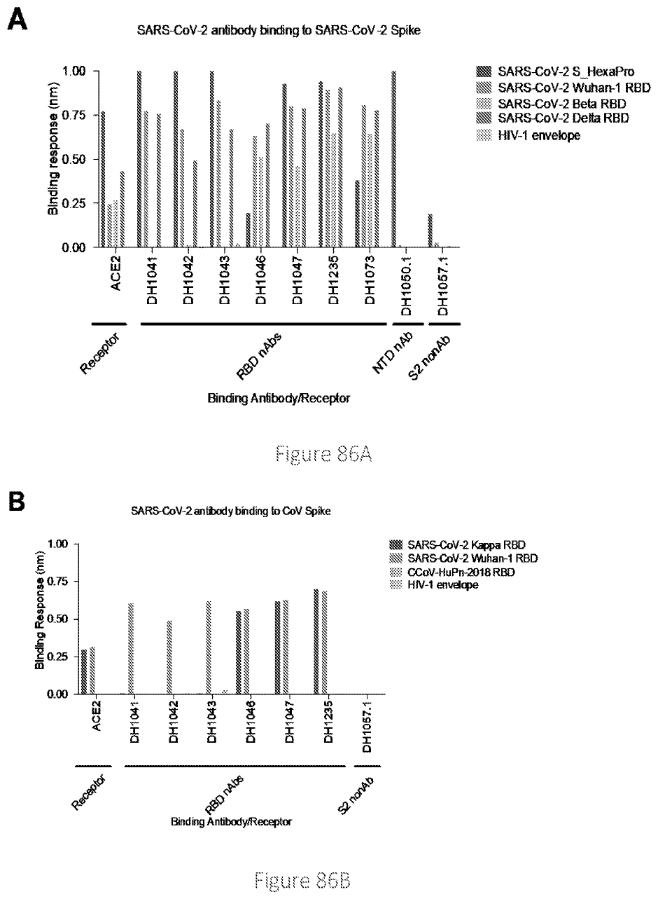

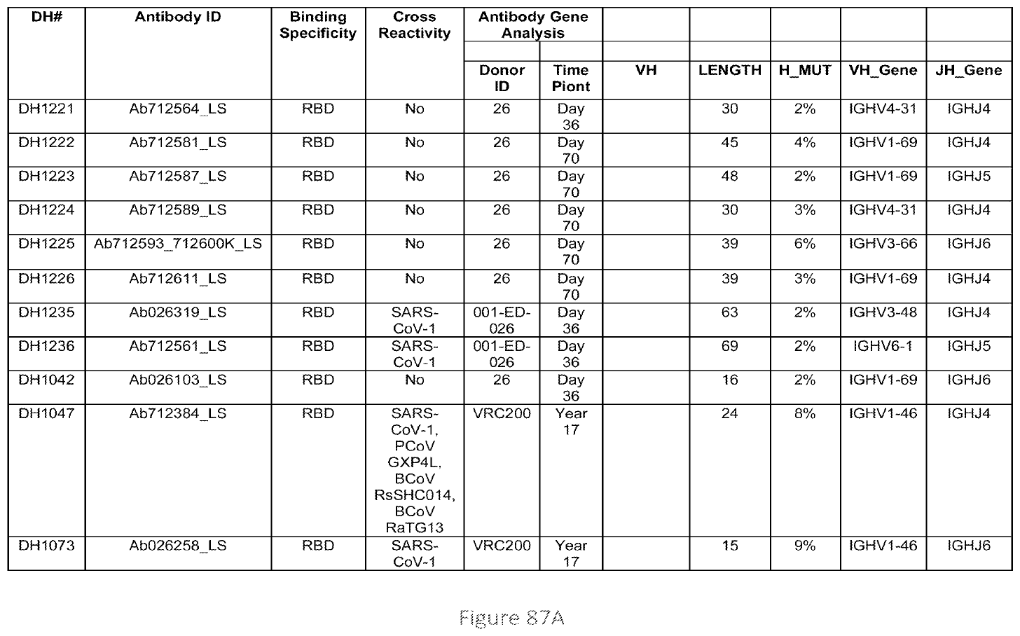

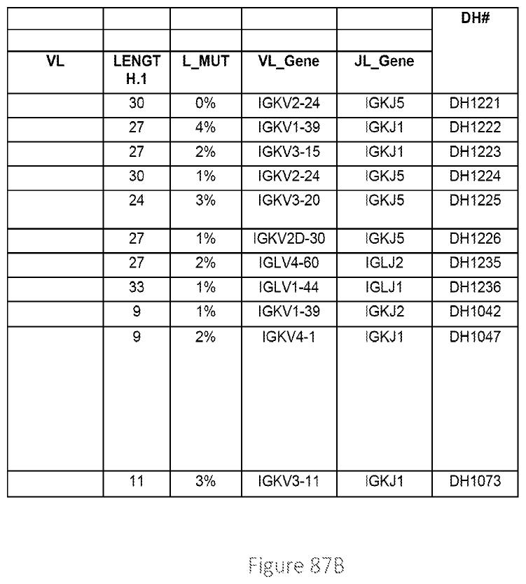

TABLE 4D
Shows summary of antibodies and gene usage. See FIG. 84, and FIG. 88.

| | Binding | Cross | Antibody gene analysis | | | | | | | | | |
|---|---|---|---|---|---|---|---|---|---|---|---|---|
| DH# | Specificity | Reactivity | VH | LENGTH | H_MUT | VH_Gene | JH_Gene | VL | LENGTH.1 | L_MUT | VL_Gene | JL_Gene |

| | | | | | | | | | | |
|---|---|---|---|---|---|---|---|---|---|---|
| DH | RBD | SARS- | 63 | 2% | IGHV3-48 | IGHJ4 | 27 | 2% | IGLV4-60 | IGLJ2 |
| 1235 | | CoV-1 | | | | | | | | |
| DH | RBD | SARS- | 69 | 2% | IGHV6-1 | IGHJ5 | 33 | 1% | IGLV1-44 | IGLJ1 |
| 1236 | | CoV-1 | | | | | | | | |
| DH | RBD | No | 16 | 2% | IGHV1-69 | IGHJ6 | 9 | 1% | IGKV1-39 | IGKJ2 |
| 1042 | | | | | | | | | | |
| DH | RBD | SARS- | 24 | 8% | IGHV1-46 | IGHJ4 | 9 | 2% | IGKV4-1 | IGKJ1 |
| 1047 | | CoV-1, | | | | | | | | |
| | | PCoV | | | | | | | | |
| | | GXP4L, | | | | | | | | |
| | | BCoV | | | | | | | | |
| | | RsSHC014, | | | | | | | | |
| | | BCoV | | | | | | | | |
| | | RaTG13 | | | | | | | | |
| DH | RBD | SARS- | 15 | 9% | IGHV1-46 | IGHJ6 | 11 | 3% | IGKV3-11 | IGKJ1 |
| 1073 | | CoV-1 | | | | | | | | |

Tables 4A, 4B, 4C and 4D are referred collectively as Table 4.

TABLE 5
Summary listing of antibodies and corresponding sequences (See FIG. 20 and 21).

| | DH | Ab ID | Heavy chain ID | Light chain ID |
|---|---|---|---|---|
| 1 | 1041 | Ab026124_LS | pH026124_LS.v2 | pL024055.v2 |
| 2 | 1042 | Ab026103_LS | pH026103_LS.v2 | pk023879.v2 |
| 3 | 1043 | Ab026116_LS | pH026116_LS.v2 | pk023888.v2 |
| 4 | 1044 | Ab026077_LS | pH026077_LS.v2 | pL024038.v2 |
| 5 | 1045 | Ab026066_LS | pH026066_LS.v2 | pL024032_L024042.v2 |
| 6 | 1046 | Ab026204_LS | pH026204_LS.v2 | pk023942.v2 |
| 7 | 1047 | Ab712384_LS | pH712384_LS.v2 | pK711897.v2 |
| 8 | 1048 | Ab712160_LS | pH712160_LS.v2 | pK711782.v2 |
| 9 | 1049 | Ab712522_G1.4A | pH712522_G1.4A | pL711764.v2 |
| 10 | 1050.2 | Ab712213_LS | pH712213_LS.v2 | pL711598.v2 |
| 11 | 1051 | Ab026013_LS | pH026013_LS.v2 | pL024005.v2 |
| 12 | 1050.1 | Ab026016_LS | pH026016_LS.v2 | pL024008.v2 |
| 13 | 1052 | Ab711733_G1.4A | pH711733_G1.4A | pK711421.v2 |
| 14 | 1053 | Ab026107_LS | pH026107_LS.v2 | pk023882.v2 |
| 15 | 1054 | Ab712151_LS | pH712151_LS.v2 | pL711550.v2 |
| 16 | 1055 | Ab025976_LS | pH025976_LS.v2 | pK023811.v2 |
| 17 | 1056 | Ab025988_LS | pH025988_LS.v2 | pK023817.v2 |
| 18 | 1057 | Ab025934_G1.4A | pH025934_G1.4A | pk023788.v2 |
| 19 | 1058 | Ab711725_G1.4A | pH711725_G1.4A | pK711414.v2 |

TABLE 6
Summary listing of antibodies and corresponding
VH and VL (See FIG. 20 and 21).

| | | | VHeavy | VLight |
|---|---|---|---|---|
| | DH | Ab ID | chain ID | chain ID |
| 1 | 1041 | Ab026124_LS | H026124_{—} | L024055 |
| 2 | 1042 | Ab026103_LS | H026103_{—} | K023879 |
| 3 | 1043 | Ab026116_LS | H026116_{—} | K023888 |
| 4 | 1044 | Ab026077_LS | H026077_{—} | pL024038 |
| 5 | 1045 | Ab026066_LS | H026066_{—} | L024032_L024042 |
| 6 | 1046 | Ab026204_LS | H026204_{—} | K023942 |
| 7 | 1047 | Ab712384_LS | H712384_{—} | K711897 |
| 8 | 1048 | Ab712160_LS | H712160_{—} | K711782 |
| 9 | 1049 | Ab712522_G1.4A | H712522_{—} | L711764 |
| 10 | 1050.2 | Ab712213_LS | H712213_{—} | L711598 |
| 11 | 1051 | Ab026013_LS | H026013_{—} | L024005 |
| 12 | 1050.1 | Ab026016_LS | H026016_{—} | L024008 |
| 13 | 1052 | Ab711733_G1.4A | H711733_{—} | K711421 |
| 14 | 1053 | Ab026107_LS | H026107_{—} | K023882 |
| 15 | 1054 | Ab712151_LS | H712151_{—} | L711550 |
| 16 | 1055 | Ab025976_LS | H025976_{—} | K023811 |
| 17 | 1056 | Ab025988_LS | H025988_{—} | K023817 |
| 18 | 1057 | Ab025934_G1.4A | H025934_{—} | K023788 |
| 19 | 1058 | Ab711725_G1.4A | H711725_{—} | K711414 |

Table 7 shows summary of cross-competition of RBD and NTD Abs, where (−) means a ternary complex is formed, i.e, no competition. (+) means a complex is not formed, indicating no binding due to competition.

| | 712160 | 712213 | 026016 | 026013 | 711733 |
|---|---|---|---|---|---|
| MAb | (DH1048) | (DH1050.2) | (DH1050.1) | (DH1051) | (DH1052) |
| 026124 | − | − | − | − | − |
| (DH1041) | | | | | |
| 026116 | − | − | − | − | − |
| (DH1043) | | | | | |
| 026077 | − | − | − | − | − |
| (DH1044) | | | | | |
| 026204 | − | − | − | − | − |
| (DH1046) | | | | | |
| 712384 | − | − | − | − | − |
| (DH1047) | | | | | |

Table 8 shows summary of cross-competition of RBD antibodies, where (−) means a ternary complex is formed, i.e, no competition. (+) means a complex is not formed, indicating no binding due to competition.

| | 026124 | 026116 | 026077 | 026204 | 712384 |
|---|---|---|---|---|---|
| MAb | (DH1041) | (DH1043) | (DH1044) | (DH1046) | (DH1047) |
| 026124 | + | + | + | − | − |
| (DH1041) | | | | | |
| 026116 | + | + | + | − | − |
| (DH1043) | | | | | |
| 026077 | + | + | + | − | − |
| (DH1044) | | | | | |
| 026204 | − | − | − | + | + |
| (DH1046) | | | | | |
| 712384 | − | − | − | + | + |
| (DH1047) | | | | | |

Table 9 shows summary of Cross-competition of NTD Abs, where (−) means a ternary complex is formed, i.e, no competition. (+) means a complex is not formed, indicating no binding due to competition.

| | 712160 | 712213 | 026016 | 026013 | 711733 |
|---|---|---|---|---|---|
| MAb | (DH1048) | (DH1050.2) | (DH1050.1) | (DH1051) | (DH1052) |
| 712160 | + | + | + | + | − |
| (DH1048) | | | | | |
| 712213 | + | + | + | + | − |
| (DH1050.2) | | | | | |
| 026016 | + | + | + | + | − |
| (DH1050.1) | | | | | |
| 026013 | + | + | + | + | − |
| (DH1051) | | | | | |
| 711733 | − | − | − | − | + |
| (DH1052) | | | | | |

TABLE 10
Summary listing of antibodies and corresponding sequences (See FIG. 22 and 23).

| DH # | Ab ID | VH ID | VL ID |
|---|---|---|---|
| DH1221 | Ab712564_LS | pH712564_LS.v2 | pK712022.v2 |
| DH1222 | Ab712581_LS | pH712581_LS.v2 | pK712032.v2 |
| DH1223 | Ab712587_LS | pH712587_LS.v2 | pK712037.v2 |
| DH1224 | Ab712589_LS | pH712589_LS.v2 | pK712038.v2 |
| DH1225 | Ab712593_712600K_LS | pH712593_H712600_LS.v2 | pK712041.v2 |
| DH1226 | Ab712611_LS | pH712611_LS.v2 | pK712052.v2 |

TABLE 11
Summary listing of antibodies and corresponding
VH and VL (See FIG. 22 and 23).

| DH # | Ab ID | VH ID | VL ID |
|---|---|---|---|
| DH1221 | Ab712564_LS | H712564 | K712022 |
| DH1222 | Ab712581_LS | H712581 | K712032 |
| DH1223 | Ab712587_LS | H712587 | K712037 |
| DH1224 | Ab712589_LS | H712589 | K712038 |
| DH1225 | Ab712593_712600K_LS | H712593_H712600_{—} | K712041 |
| DH1226 | Ab712611_LS | H712611 | K712052 |

Table 12 shows Cross-competition of RBD Abs where (−) means a ternary complex is formed, i.e, no competition. (+) means a complex is not formed, indicating blocked binding due to competition.

| MAb | DH1221 | DH1222 | DH1223 | DH1224 | DH1225 | DH1226 | DH1043 | DH1047 |
|---|---|---|---|---|---|---|---|---|
| DH1221 | + | + | + | + | + | + | + | − |
| DH1222 | + | + | + | + | + | + | + | − |
| DH1223 | + | + | + | + | + | + | + | − |
| DH1224 | + | + | + | + | + | + | + | − |
| DH1225 | + | + | + | + | + | + | + | − |
| DH1226 | + | + | + | + | + | + | + | − |
| DH1043 | + | + | + | + | + | + | + | − |
| DH1047 | − | − | − | − | − | − | − | + |

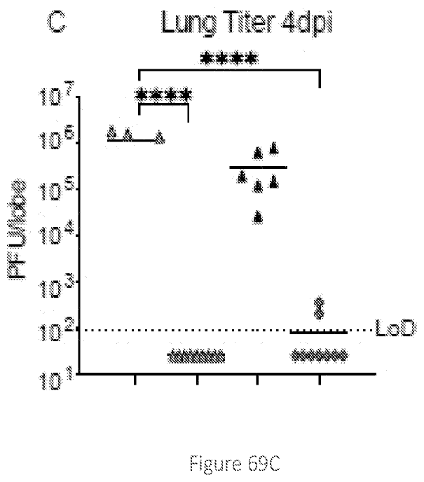

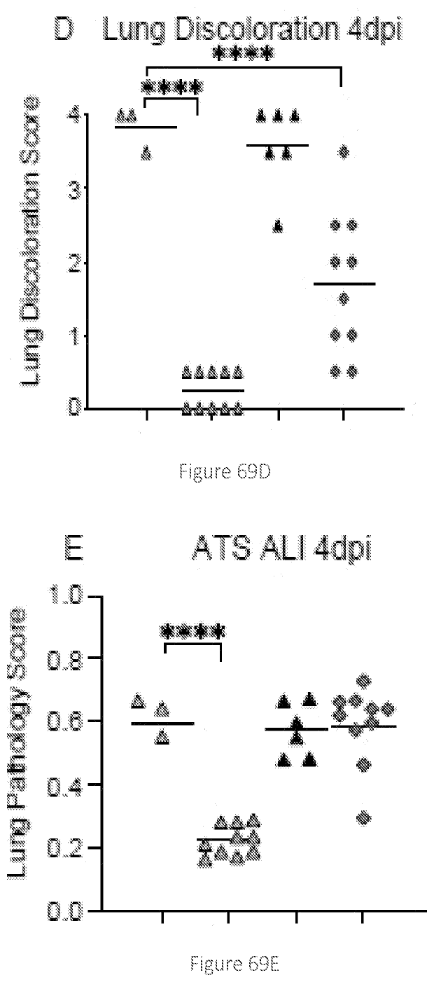

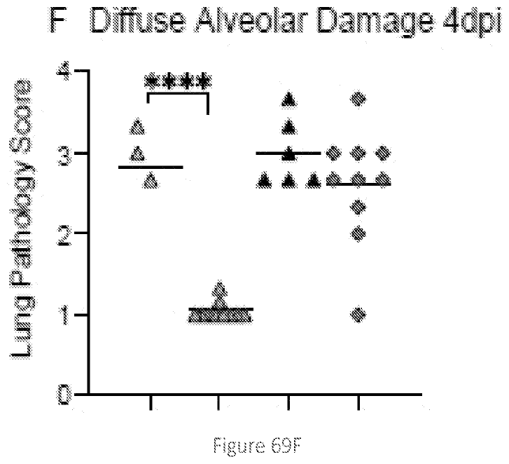

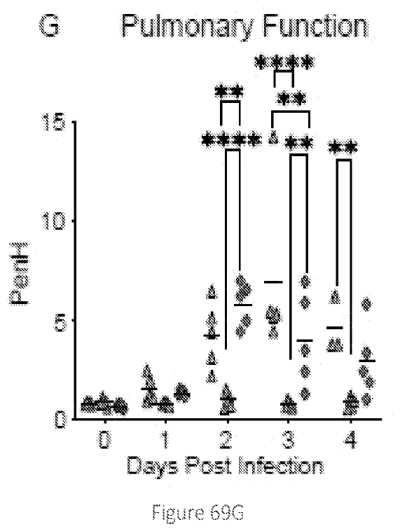

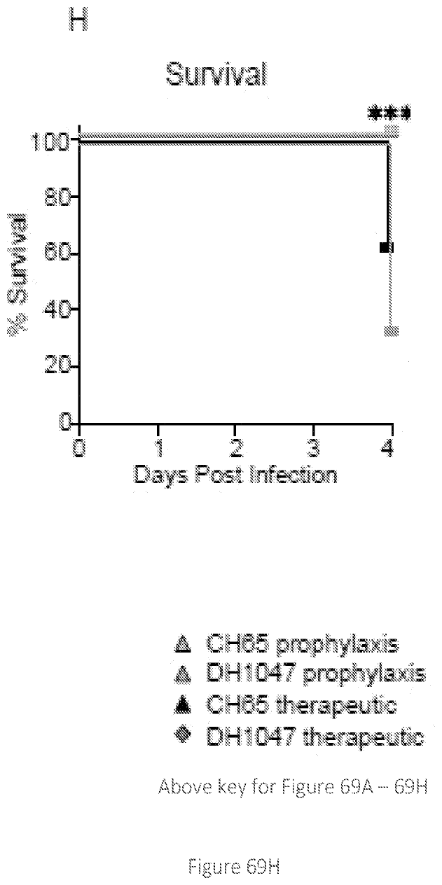

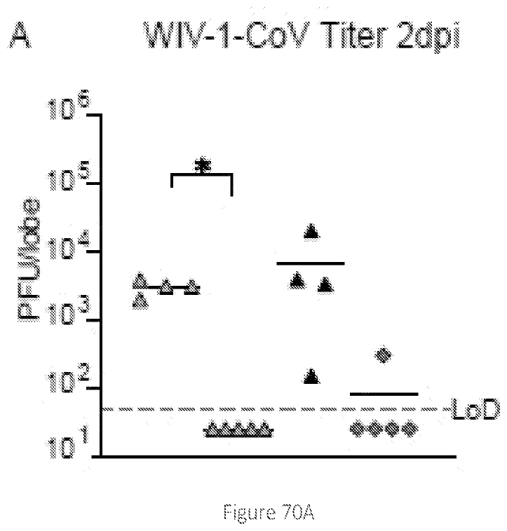

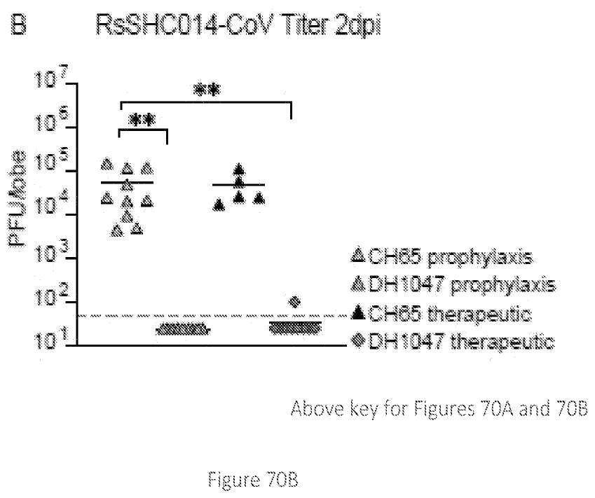

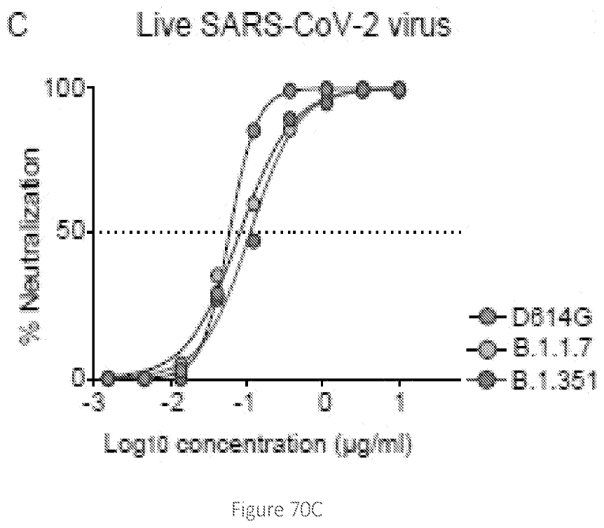

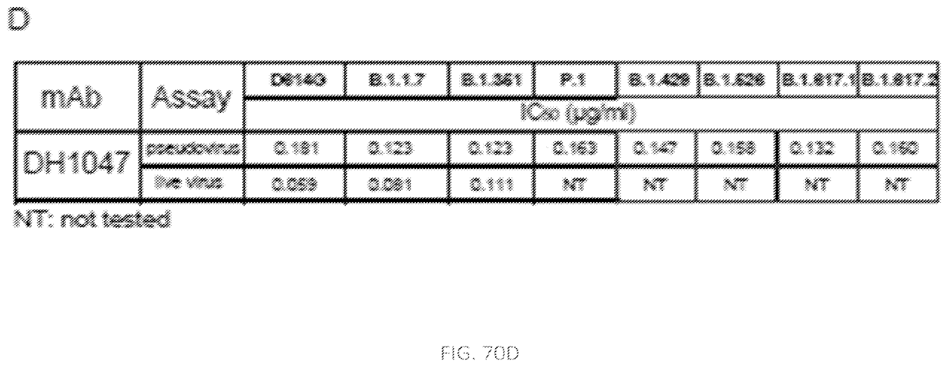

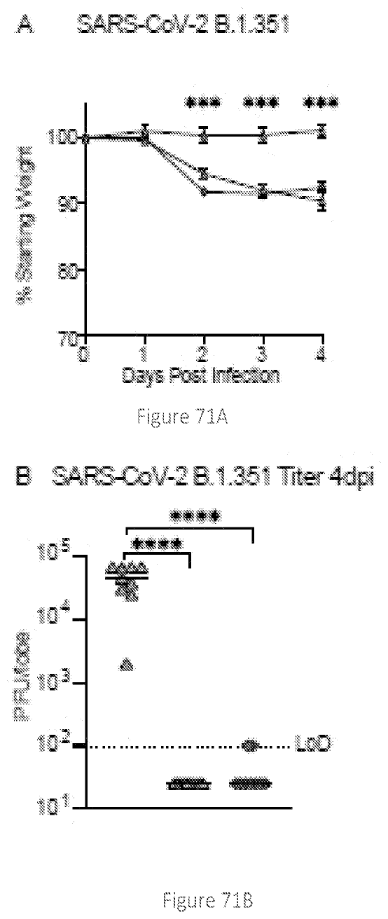

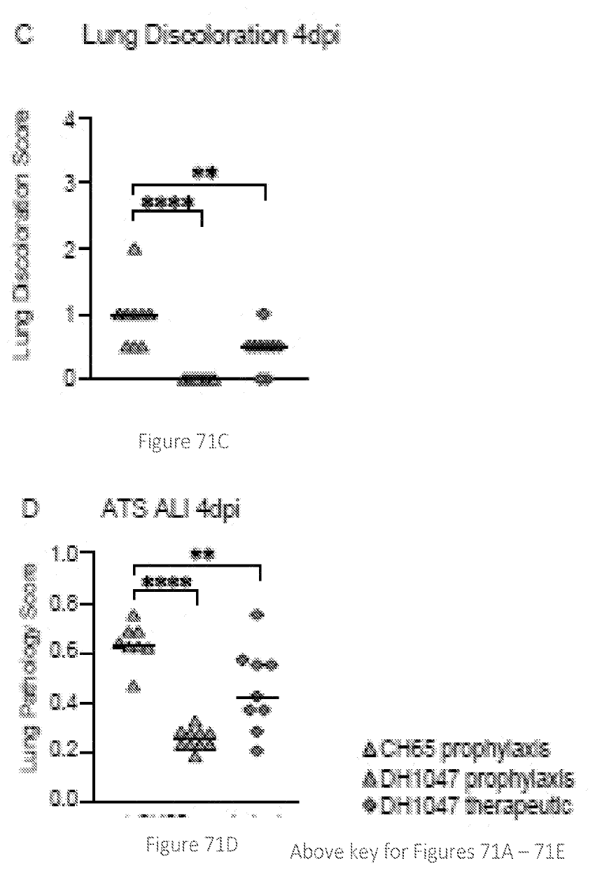

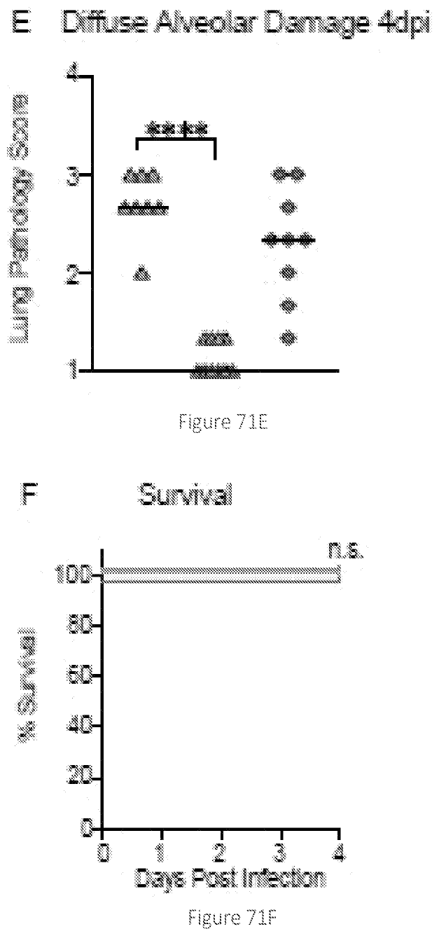

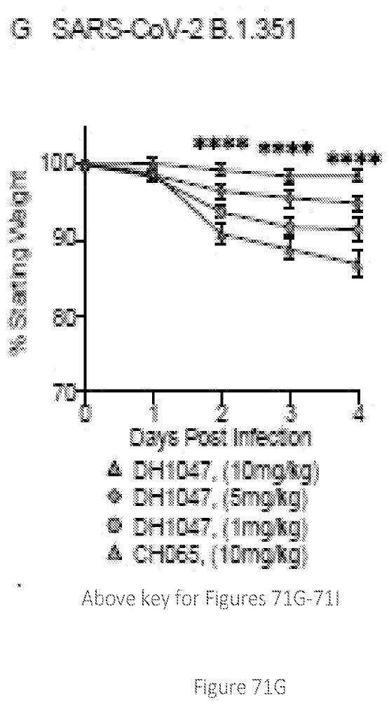

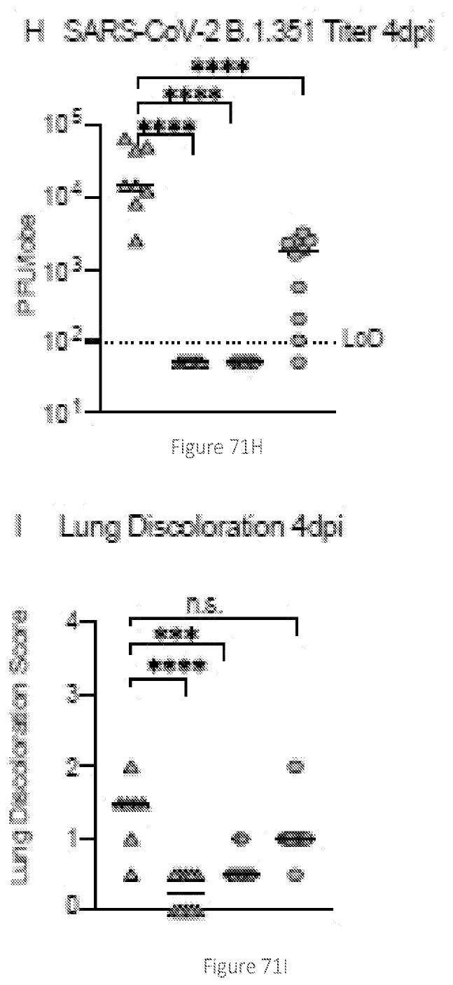

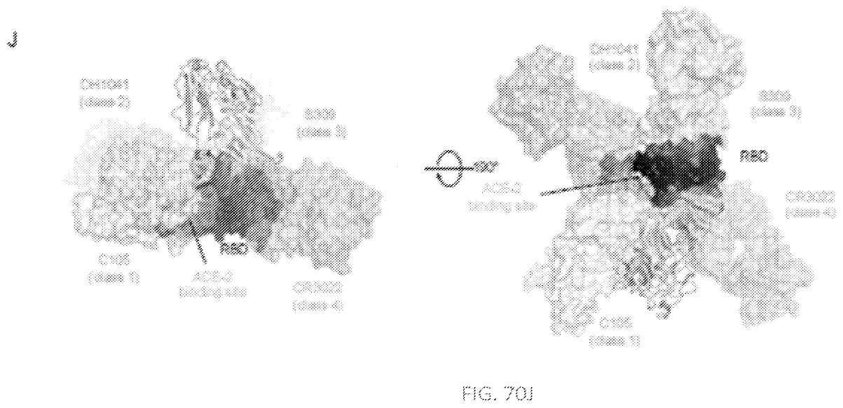

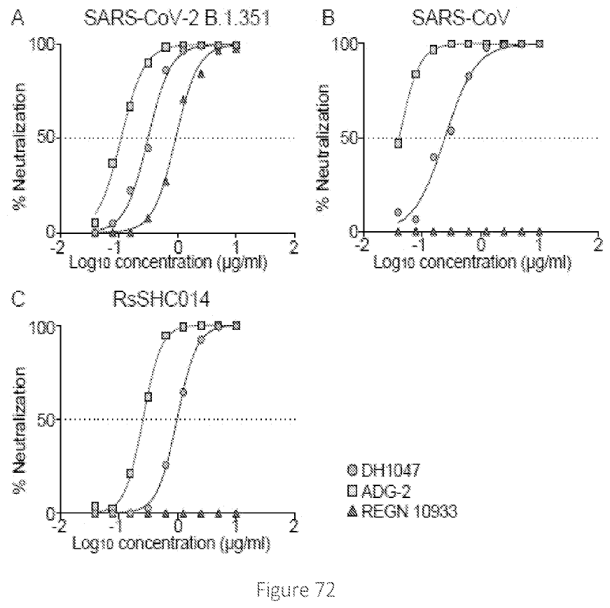

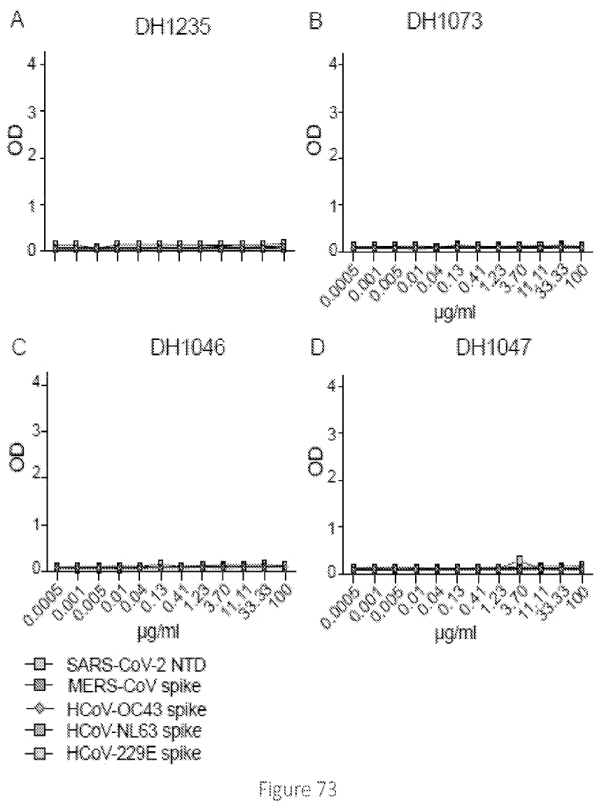

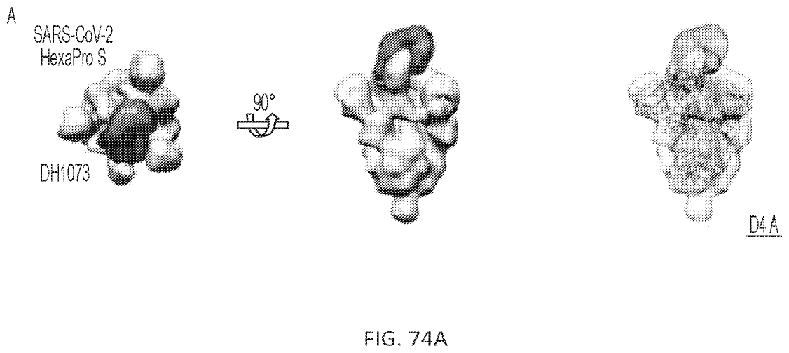

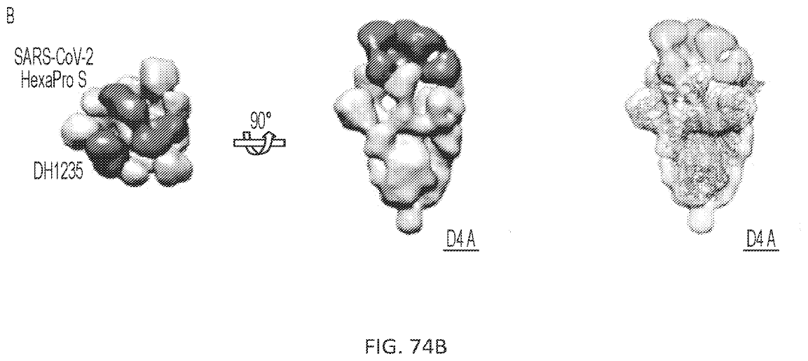

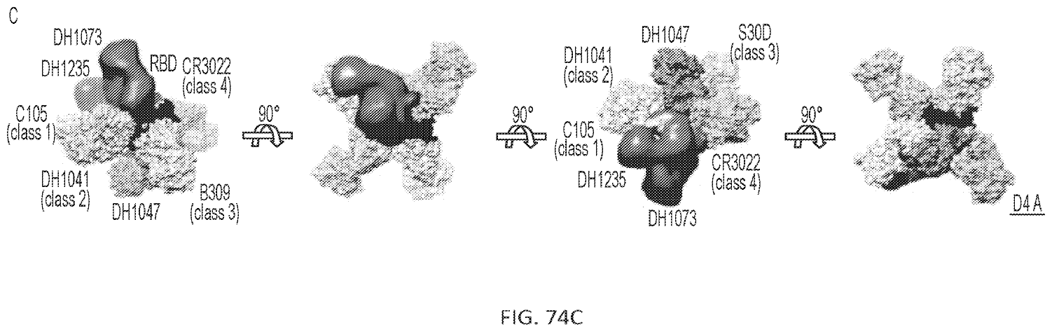

FIG. 74 SPR competition experiments with DH1047, DH1046, DH1073 and DH1235. DH1047, DH1046, and DH1235 were outcompeted by one another (FIG. 74D), whereas DH1073 was not, indicating that DH1073 targets a distinct non-cross-competing epitope. Thus, in certain non-limiting embodiments, antibodies targeting non-cross-competing epitopes can be used in a combination treatment. In non-limiting embodiments, the combination comprises DH1073 antibody or antigen binding fragment thereof, and any other non-cross-competing antibody, e.g. without limitation DH1047, DH1046, or DH1042.

### EXAMPLES

### Example 1-Antibody Isolation

Antibodies were isolated and lineages were identified as previously published.

1. Liao H X, Levesque M C, Nagel A, et al. High-throughput isolation of immunoglobulin genes from single human B cells and expression as monoclonal antibodies. J Virol Methods. 2009: 158 (1-2): 171-179.

2. Kepler T B, Munshaw S, Wiehe K, et al. Reconstructing a B-Cell Clonal Lineage. II. Mutation, Selection, and Affinity Maturation. Front Immunol. 2014:5:170.

FIGS. 20-25 and Examples DH1041 and DH043 show non-limiting embodiments of nucleic acids encoding antibodies of the invention.

In one embodiment, the IgG constant region comprises the LS mutation. Embodiments also comprise additional variants of the Fc portion of the antibody. See Maeda et al. MAbs. 2017 July: 9 (5): 844-853. Published online 2017 Apr. 7, PMID: 28387635.

For L S mutation See e.g. Gaudinski M R, Coates E E, Houser K V, Chen G L, Yamshchikov G, Saunders J G, et al. (2018) Safety and pharmacokinetics of the Fc-modified HIV-1 human monoclonal antibody VRCOILS: A Phase 1 open-label clinical trial in healthy adults. PLOS Med 15 (1): e1002493, https://doi.org/10.1371/journal.pmed. 100249.

### Example 2-Binding and Neutralization Assays

ELISA binding assays were characterized essentially as described in Edwards et al. Cold sensitivity of the SARS-COV-2 spike ectodomain, Jul. 13, 2020 doi: 10.1101/2020.07.12.199588, published on biorxiv.org--content/10.1101/2020.07.12.199588v1).

In some embodiments of the PRNT assay, Vero cells were used. In some embodiments of the pseudovirus assay, 293T/ACE2 (293T that overexpressed human ACE2) were used.

### Example 3-Cross Blocking and Cross Reactivity to Other Coronaviruses

Several of the antibodies do not cross-block each other in experiment of surface plasmon resonance assays. Binding and cross blocking analysis was conducted by standard SPR methods, where a first antibody is immobilized on the surface, an antigen is floated over and second antibody is floated over the complex. Results from these experiments are shown in Tables 7-9, 12 as a qualitative summary.

Table 8 shows the RBD antibodies that do or do not cross-block each other, Table 9 shows the NTD antibodies that do or do not block each other, and Table 7 shows the RBD and NTD antibodies that do not block each other. Those antibodies that do not block each other can be used in combinations of neutralizing antibodies for either prevention of SARS-COV-2 infection or for treatment of COVID-1 disease. Other antibodies listed in Tables 3-6, 10 or 11 that are not in the Tables 7-9, can also be combined for prevention or treatment by performing the same types of blocking experiments with surface plasmon resonance.

### Example 4-Animal Studies

Any other suitable coronavirus animal model can be used to characterize the antibodies of the invention. In non-limiting embodiments, a mouse, hamster, or NHP animal model is used to characterize the antibodies.

Animal studies may be designed using either recombinant protein or mRNA in composition suitable for mRNA delivery.

Methods for pharmacokinetic evaluation of an antibody in vivo are well known in the art.

### Example 5 the In Vitro and In Vivo Function of SARS-COV-2 Neutralizing Antibodies

SARS-COV-2 neutralizing antibodies (NAbs) protect against COVID-19, making them a focus of vaccine design. A safety concern regarding SARS-COV-2 antibodies is whether they mediate disease enhancement. Here, we isolated potent NAbs against the receptor-binding domain (RBD) and the N-terminal domain (NTD) of SARS-COV-2 spike protein from individuals with acute or convalescent SARS-COV-2 or a history of SARS-COV-1 infection. Cryo-electron microscopy of RBD and NTD antibodies demonstrated function-specific modes of antibody binding. Select RBD NAbs also demonstrated Fc receptor-y (FcγR)-mediated enhancement of virus infection in vitro, while five non-neutralizing NTD antibodies mediated FcγR-independent in vitro infection enhancement. However, both in vitro neutralizing and infection-enhancing RBD or infection-enhancing NTD antibodies protected from SARS-COV-2 challenge in non-human primates. Thus, these in vitro assessments of enhanced antibody-mediated infection do not indicate biologically relevant in vivo infection enhancement.

### INTRODUCTION

The severe acute respiratory syndrome coronavirus 2 (SARS-COV-2) has caused a global pandemic with over 43 million cases and 1.16 million deaths (https://coronavirus.jhu.edu/). Development of combinations of neutralizing antibodies for prevention or treatment of infection can help to control the pandemic, while the ultimate solution to control the COVID-19 pandemic is a safe and effective vaccine (1. 2).

Neutralizing antibodies (NAbs) that can block viral entry are crucial for controlling virus infections (3-5). Previously reported SARS-COV and MERS-COV NAbs function by targeting the receptor-binding domain (RBD) or the N-terminal domain (NTD) of spike(S) protein to block receptor binding, or by binding on the S2 region of S protein to interfere with S2-mediated membrane fusion (6-8). Prophylactic or therapeutic use of SARS-COV-2 NAbs in non-human primates (9) or rodent models (10-12) showed protection from SARS-COV-2-induced lung inflammation and/or reduction in viral load. SARS-COV-2 NAbs reported to date predominantly target the RBD region (9. 11-25). In contrast. NTD antibodies that neutralize SARS-COV-2 are rare and of modest neutralization potency (18. 19. 26. 27).

Antibody-dependent enhancement (ADE) of infection in vitro has been reported with a number of viruses. ADE has been associated with vaccination for respiratory syncytial virus (RSV), with vaccination for dengue virus, or with dengue virus infection (28). ADE is often mediated by Fcγ receptors for IgG (FcγRs), complement receptors (CRs) or both, and is most commonly observed in cells of monocyte/macrophage and B cell lineages (29. 30). In vitro studies have demonstrated FcγR-mediated ADE of SARS-COV-1 infection of ACE2-negative cells (31-37). One group demonstrated FcγR-independent infection enhancement of SARS-CoV-1 in Vero cells, and isolated a monoclonal antibody that can induce enhanced lung viral load and pathology in vivo (38). The ability of antibodies that bind the SARS-COV-2 S protein to mediate infection enhancement in vivo is unknown but is a concern for COVID-19 vaccine development (28. 29. 39).

Here, we identified potent in vitro neutralizing RBD and NTD antibodies as well as in vitro infection-enhancing RBD and NTD antibodies from individuals infected with SARS-CoV-1 or SARS-COV-2. Negative stain electron microscopy (NSEM) and cryo-electron microscopy (cryo-EM) revealed distinct binding patterns and the precise epitopes of infection-enhancing and neutralizing antibodies. In vitro studies using human FcγR-expressing or ACE2-expressing cell lines demonstrated that the RBD antibodies mediated FcγR-dependent infection enhancement, whereas the NTD antibodies induced FcγR-independent infection enhancement. However, using monkey models of SARS-COV-2 infection, none of these infection-enhancing antibodies augmented SARS-COV-2 lung viral load, infectious virus in the lung, or lung disease pathology in vivo. Rather, the in vitro infection-enhancing antibodies prevented or reduced virus replication in cynomolgus macaques or mice challenged with SARS-COV-2. Thus, the in vitro infection enhancing activity of SARS-COV-2 RBD and NTD antibodies did not translate to in vivo relevance.

### Results

### Isolation of Infection-Enhancing SARS-COV-2 Antibodies

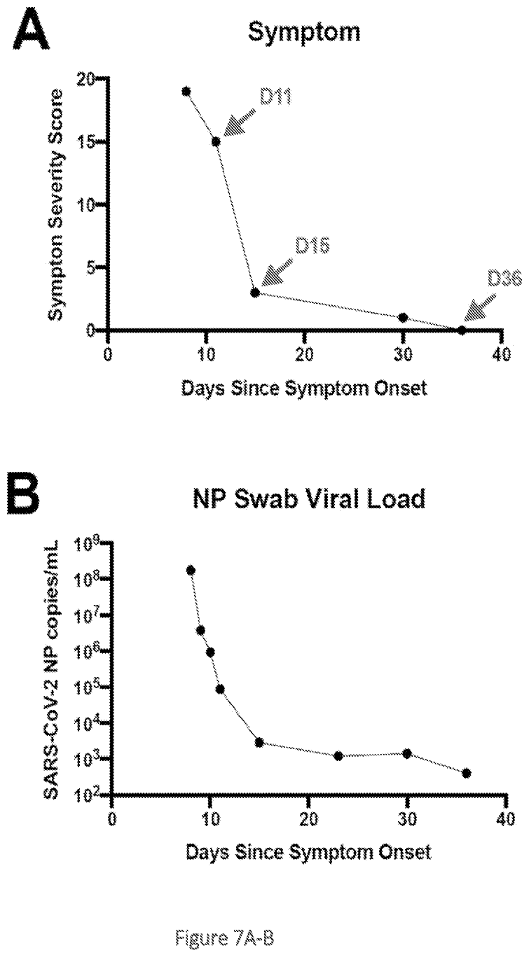

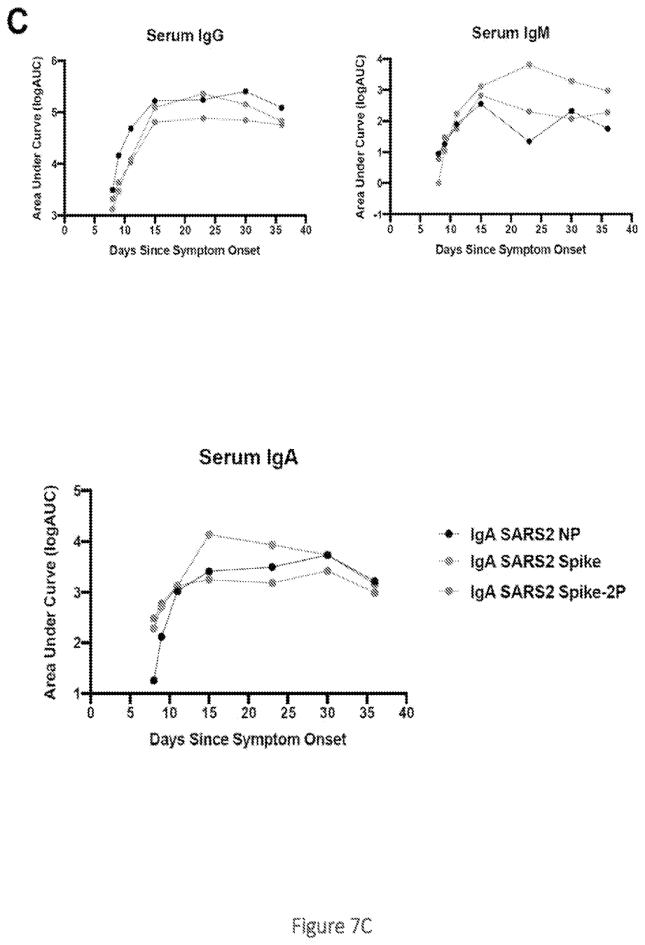

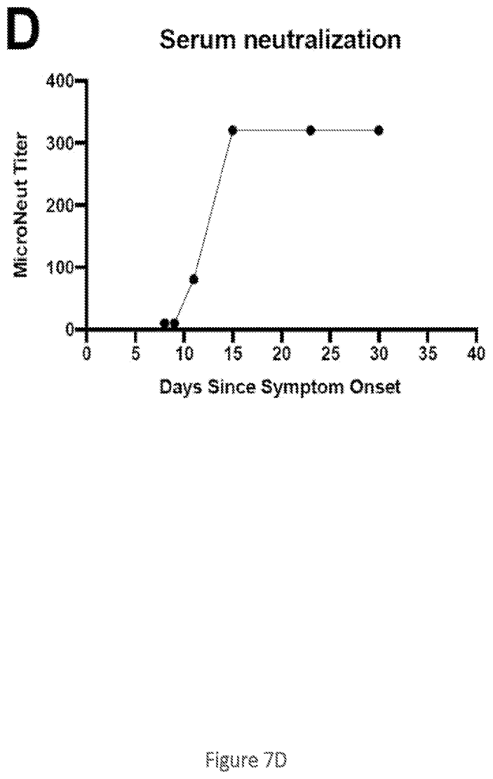

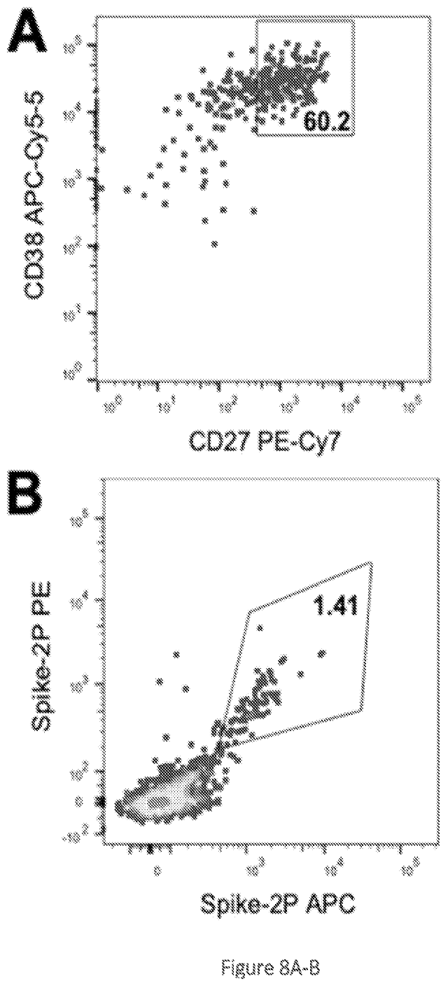

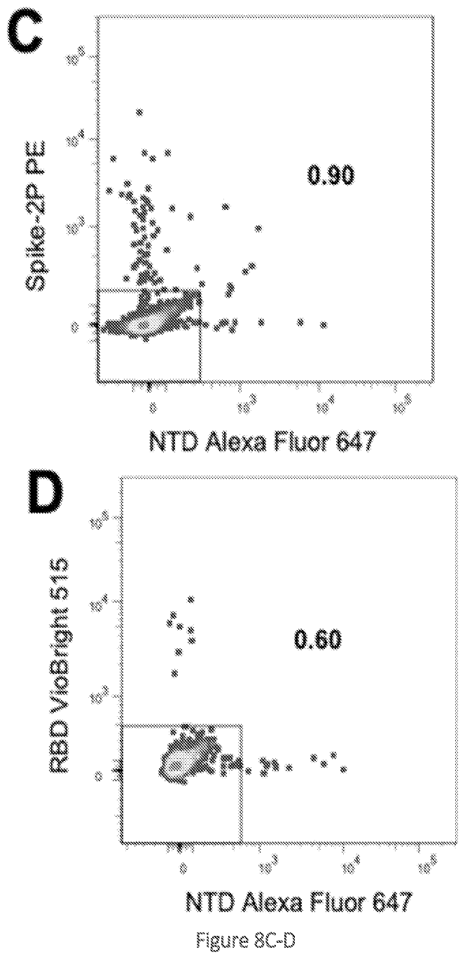

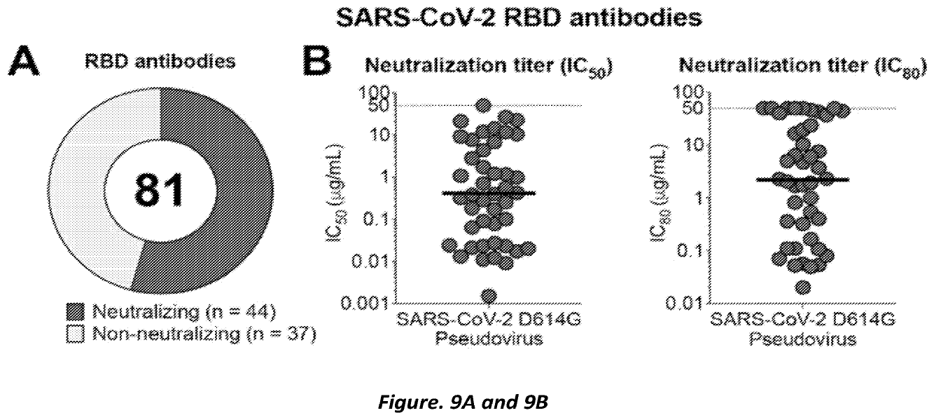

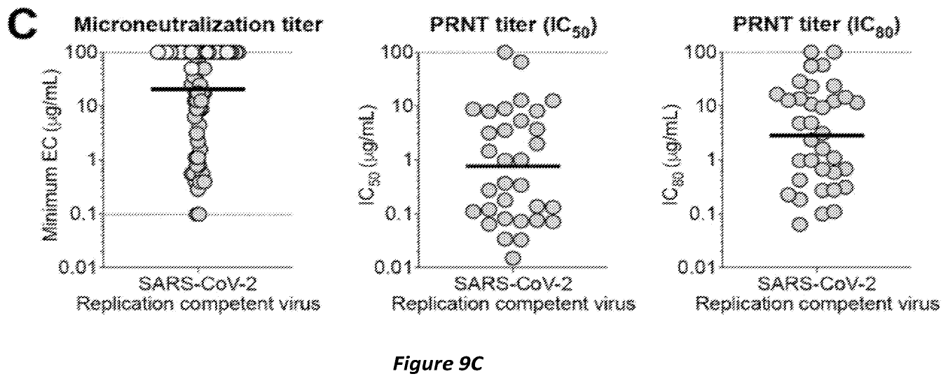

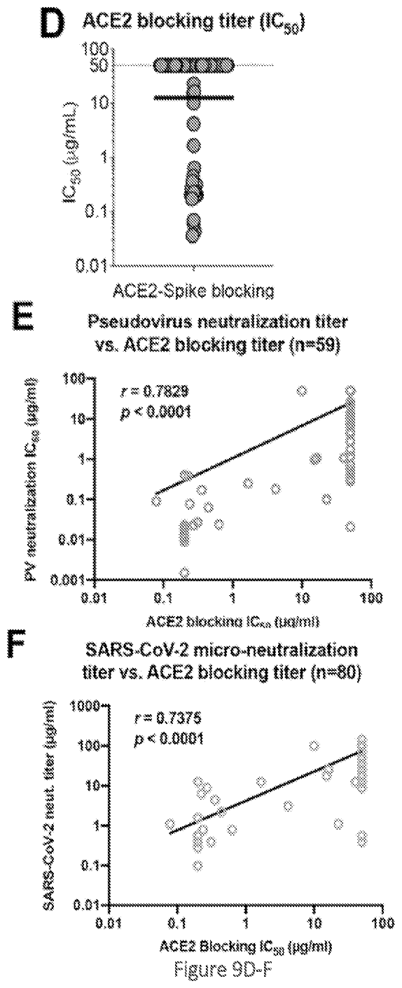

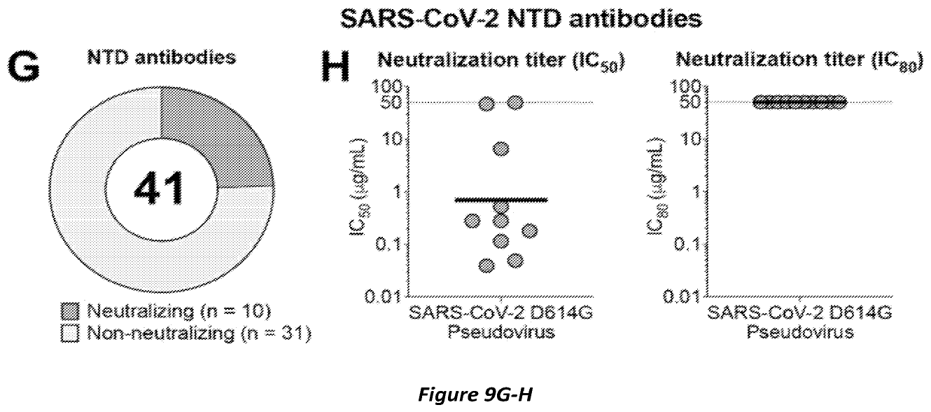

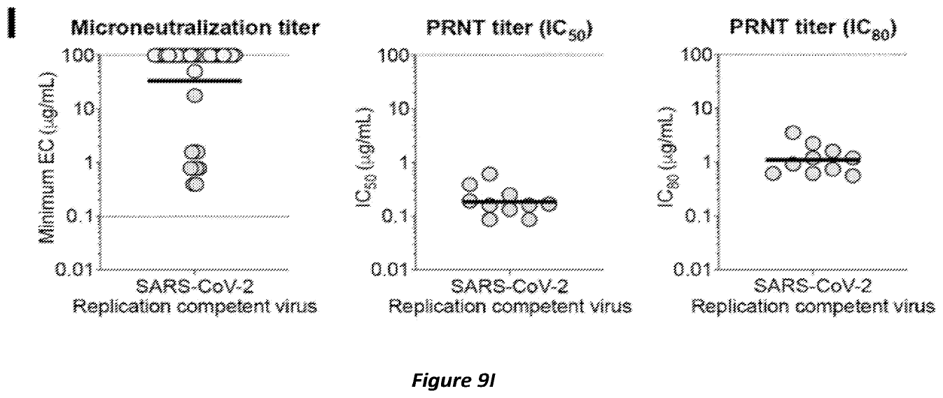

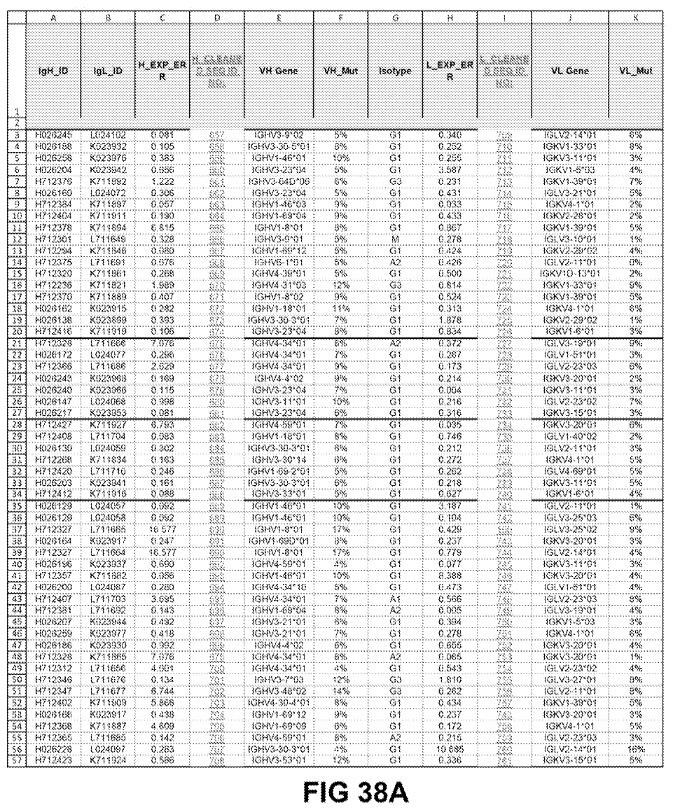

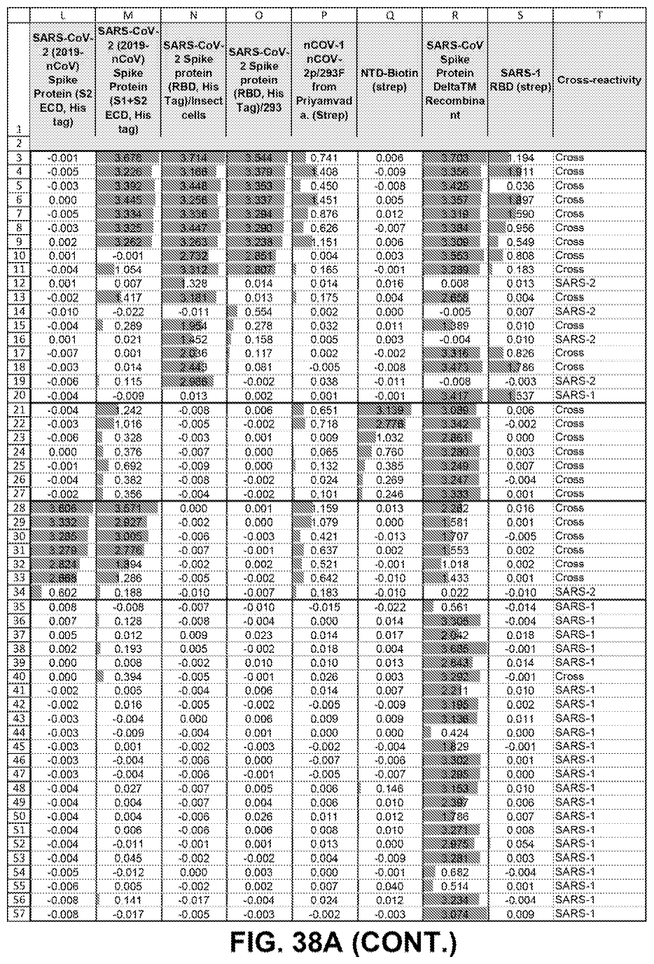

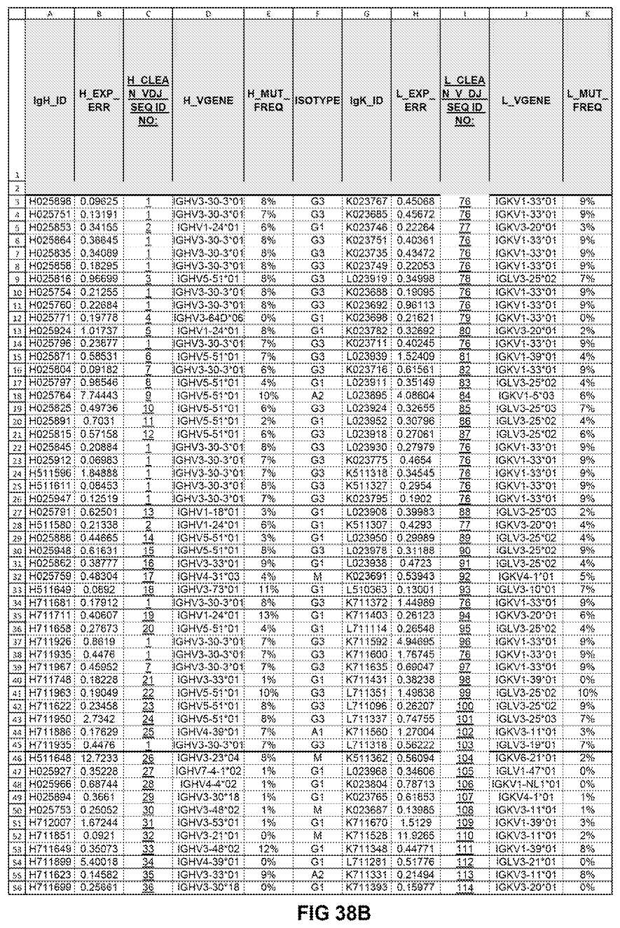

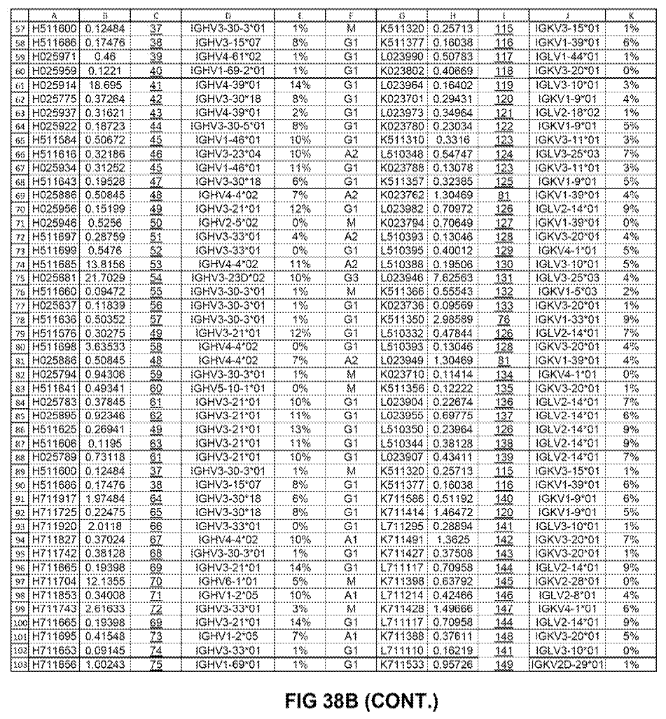

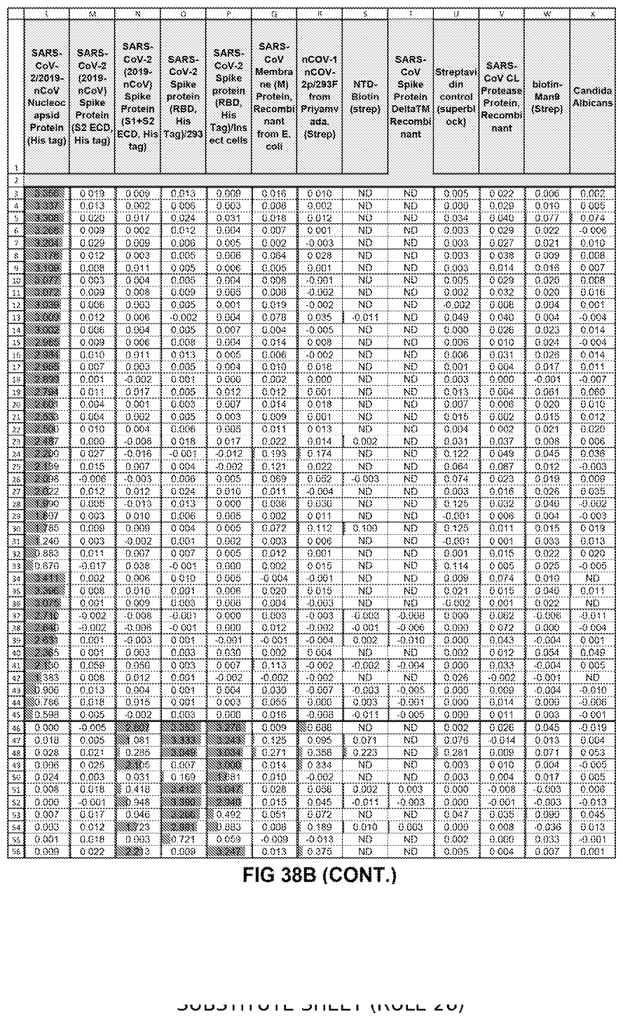

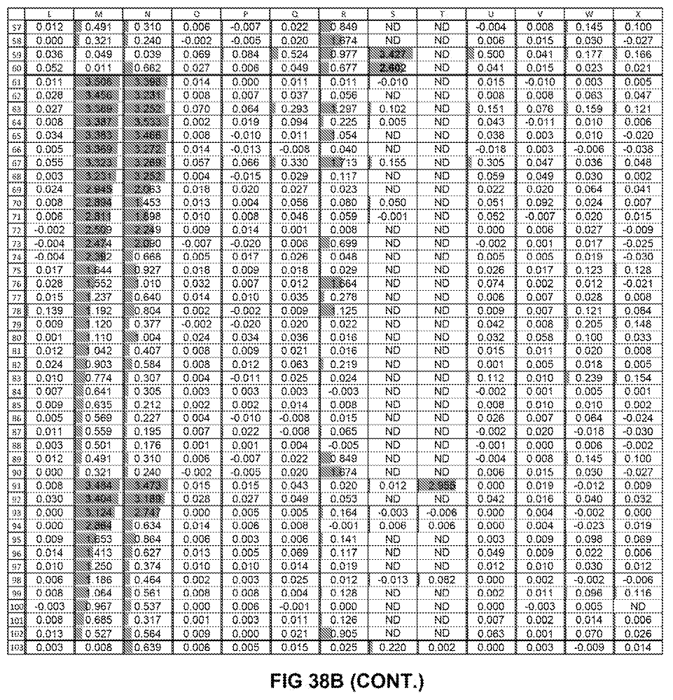

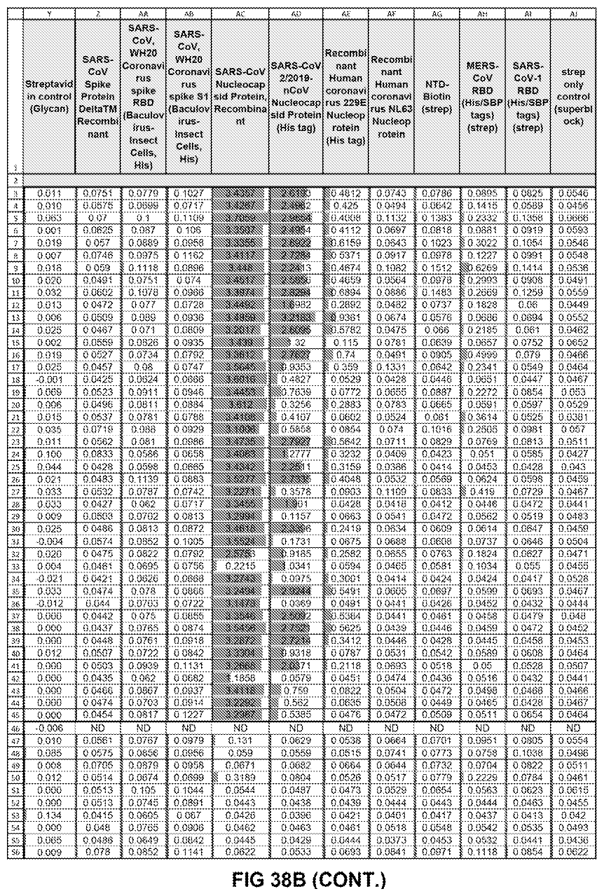

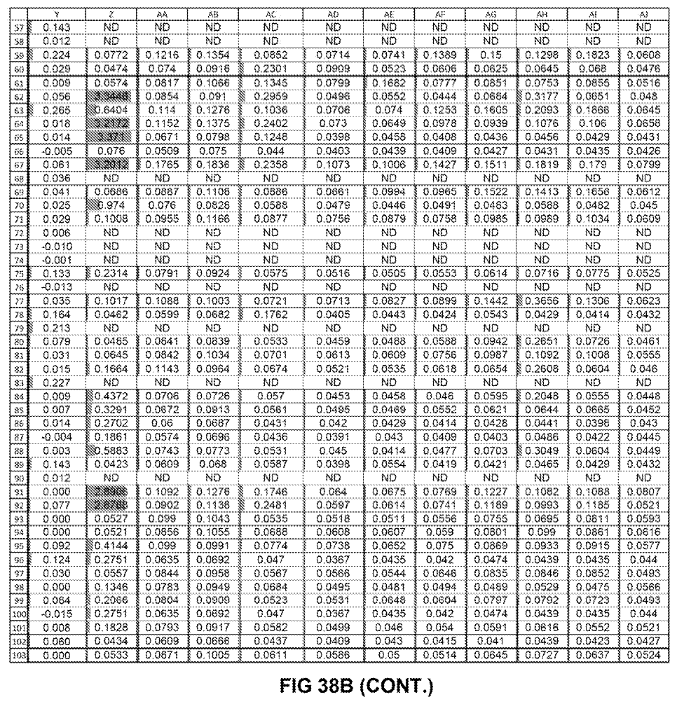

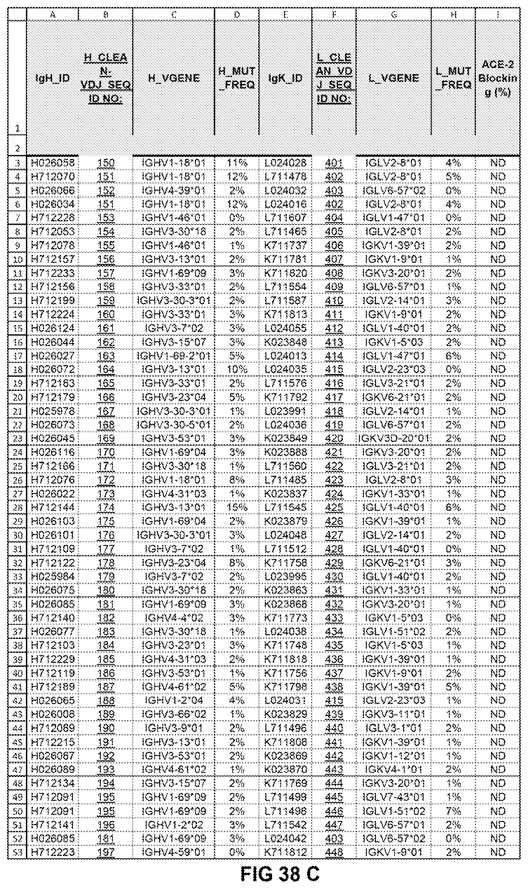

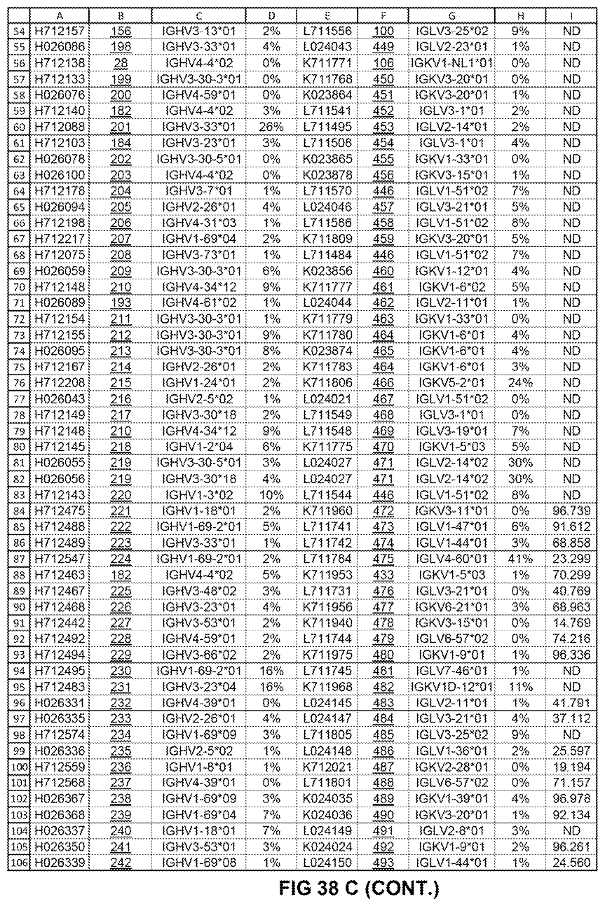

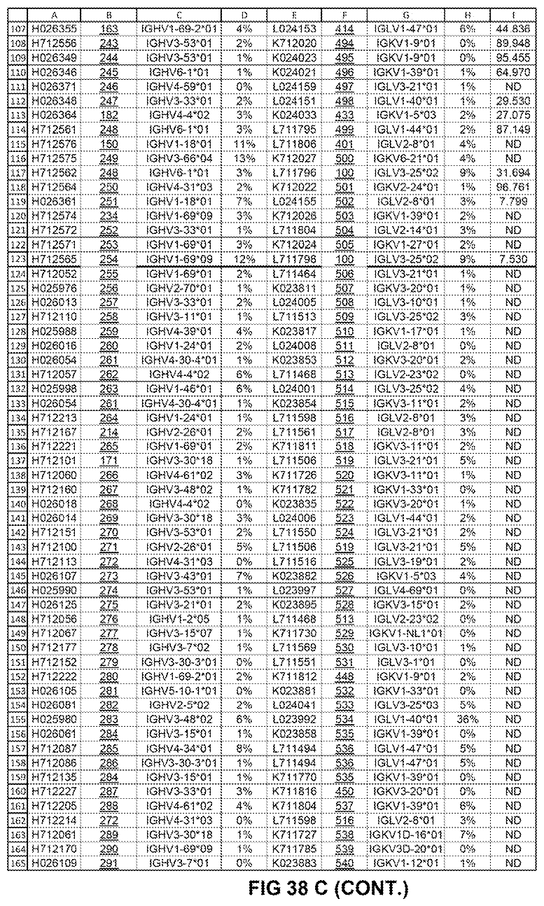

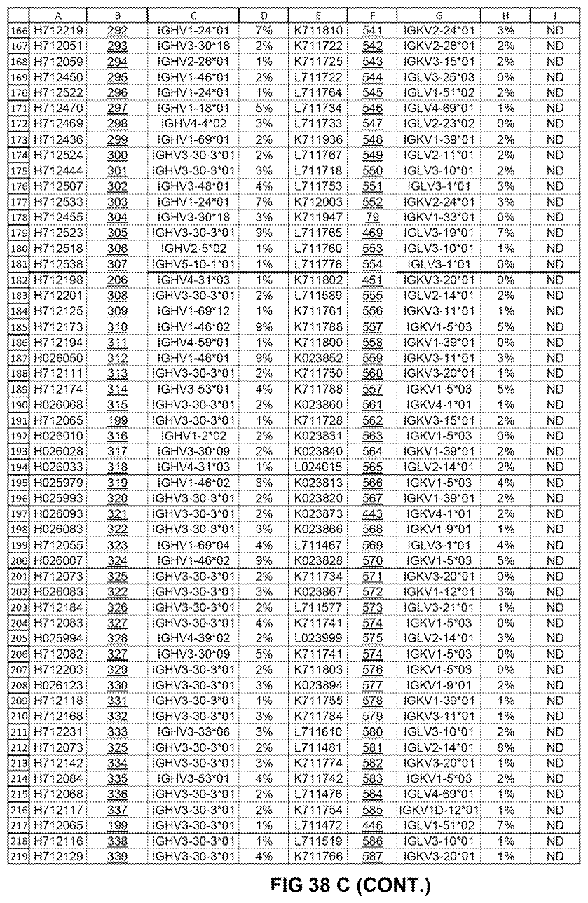

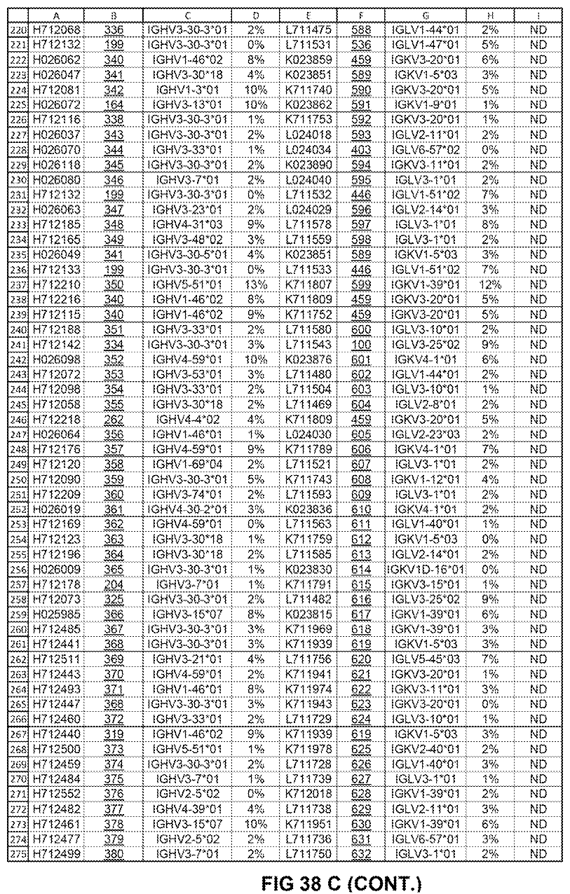

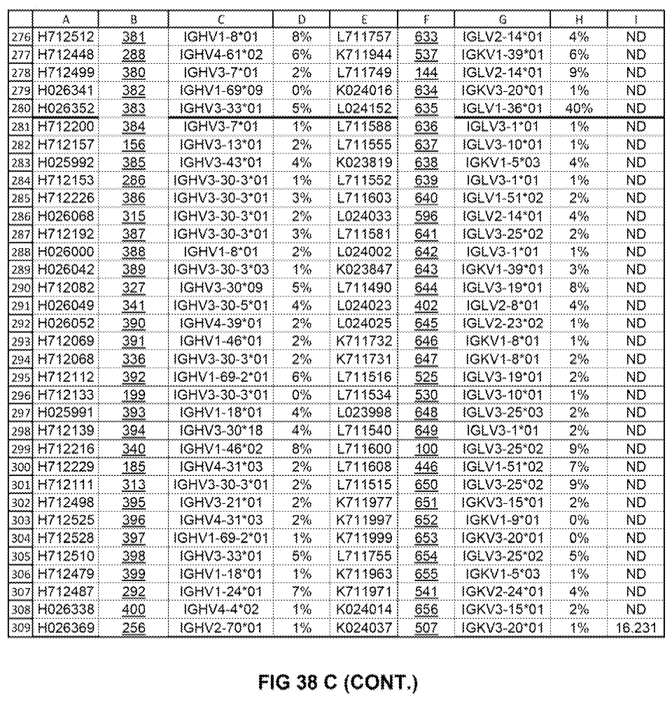

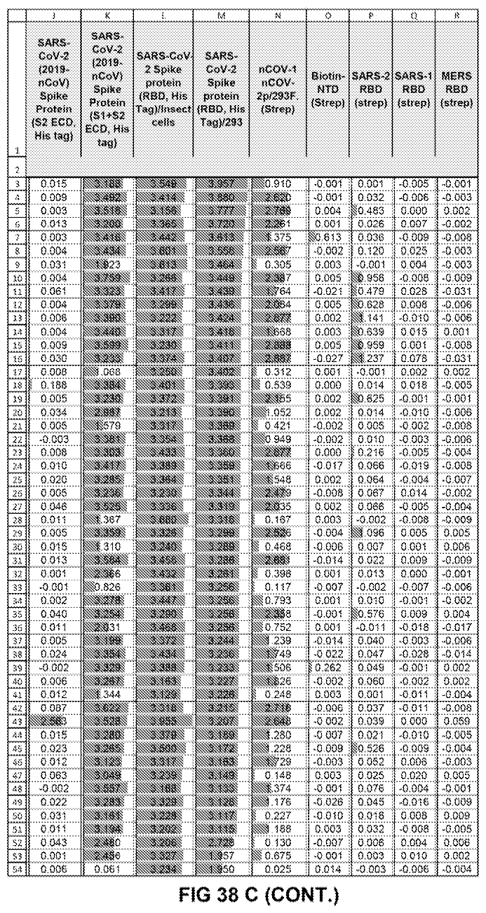

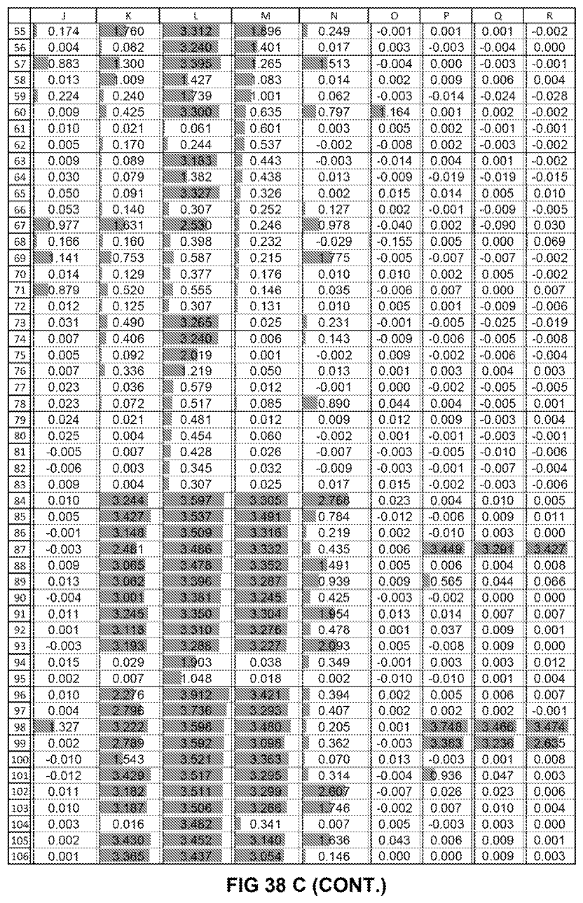

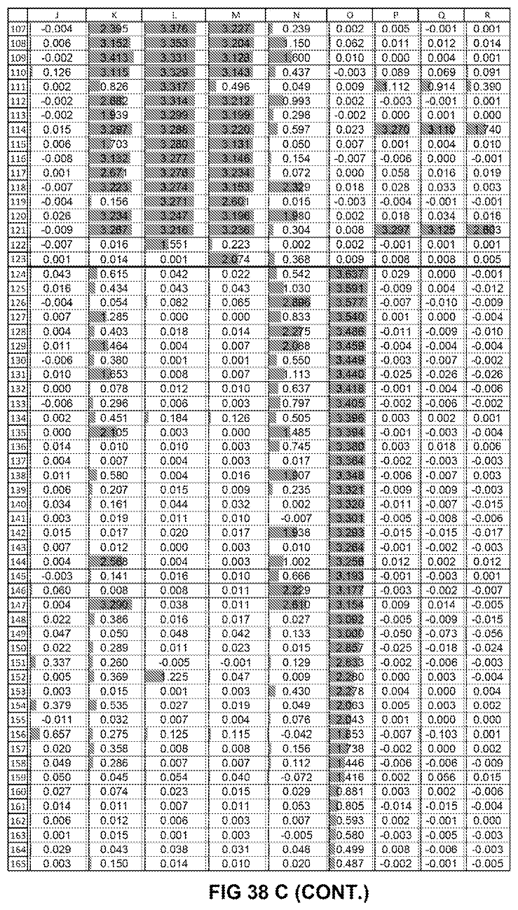

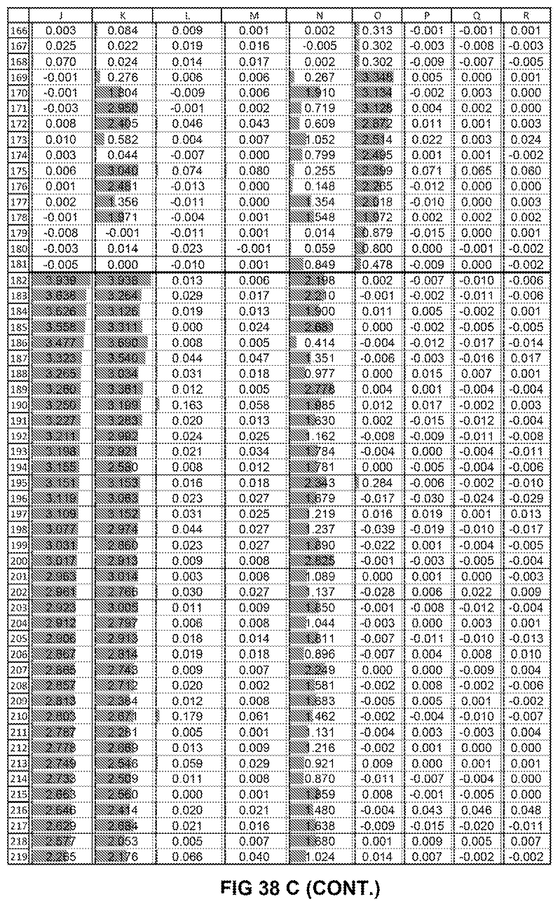

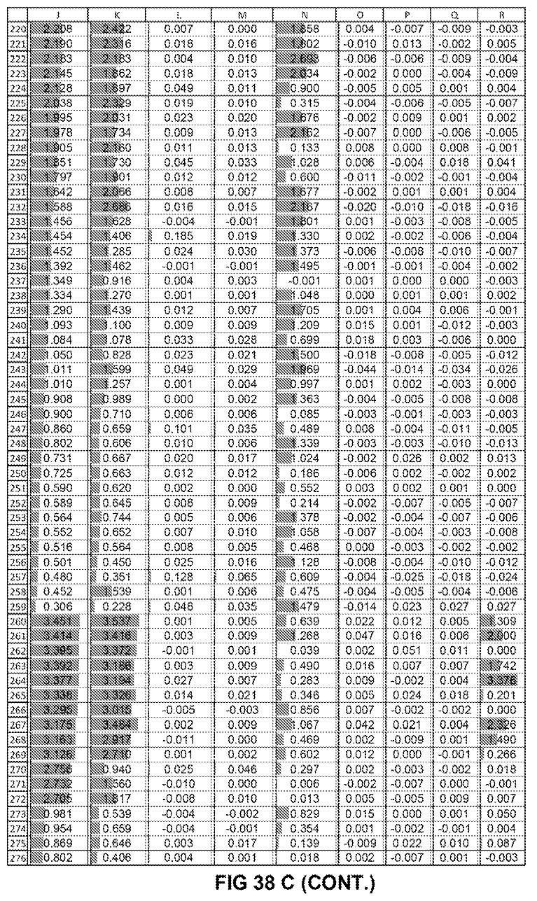

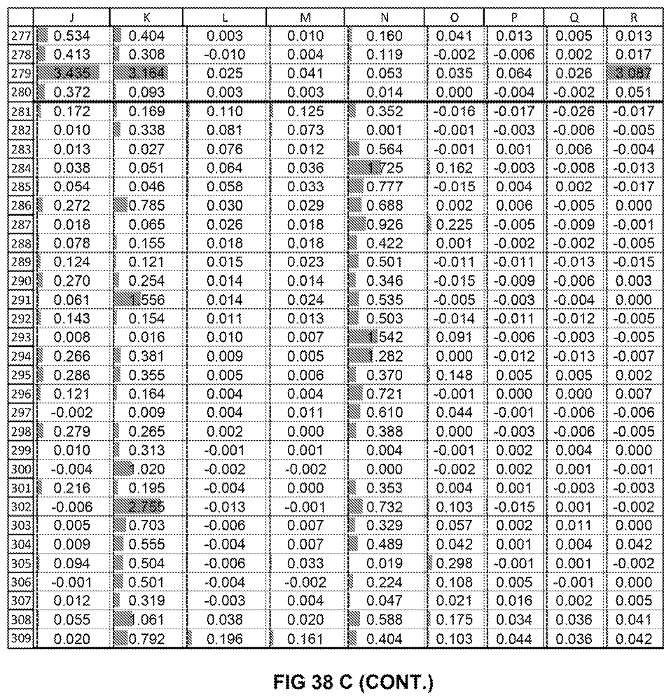

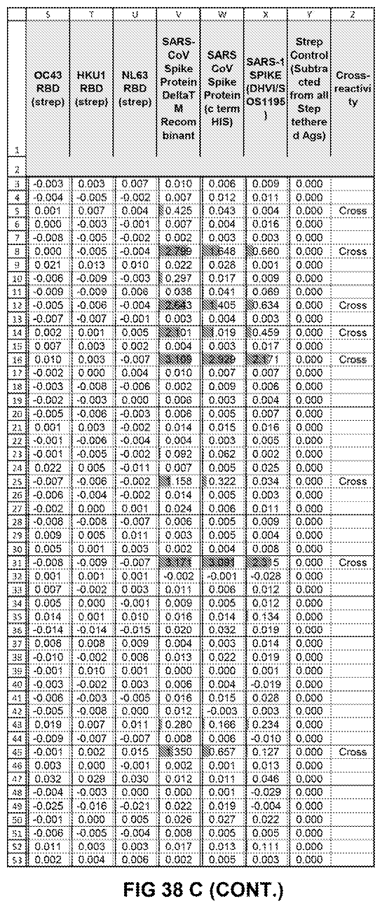

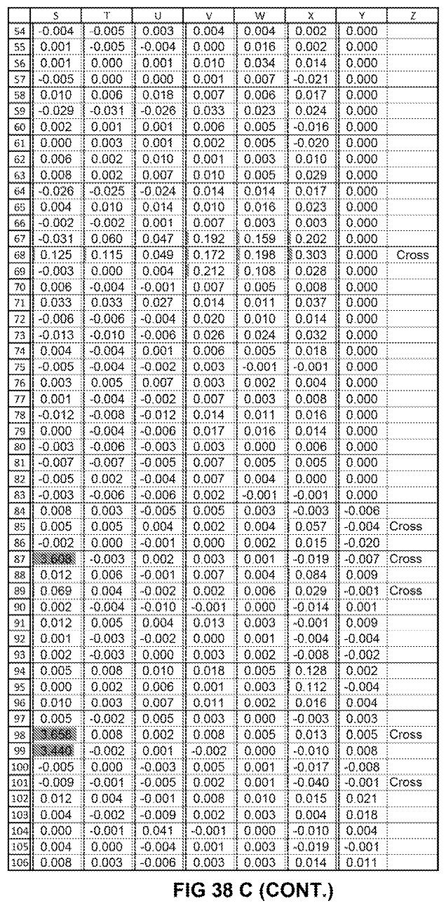

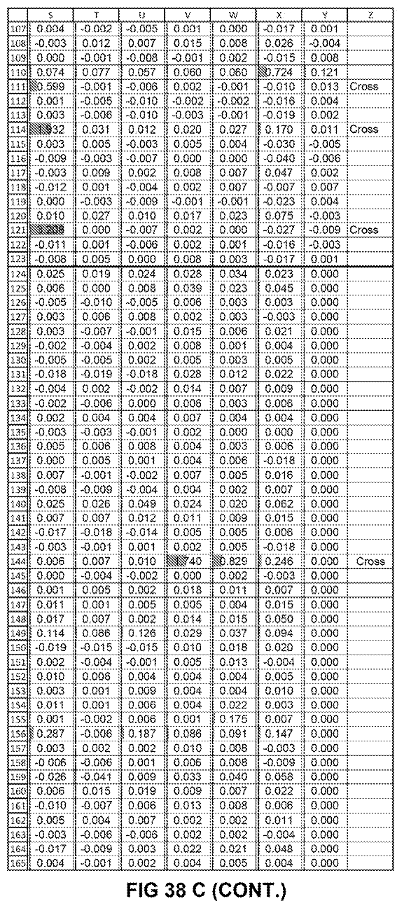

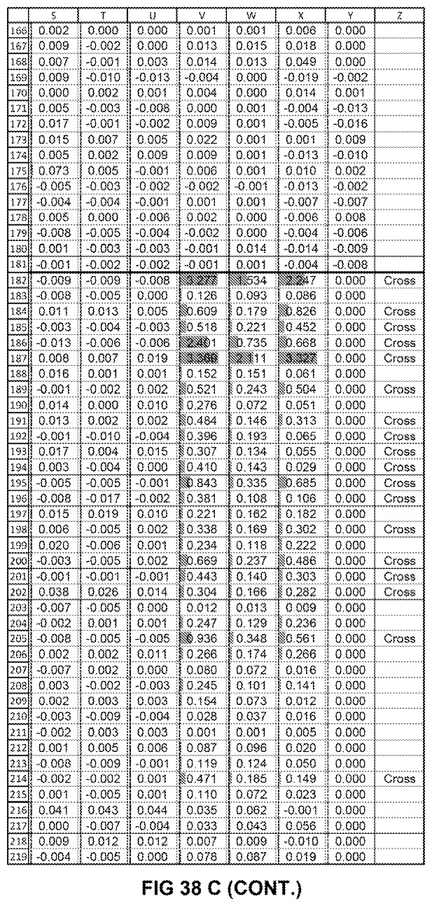

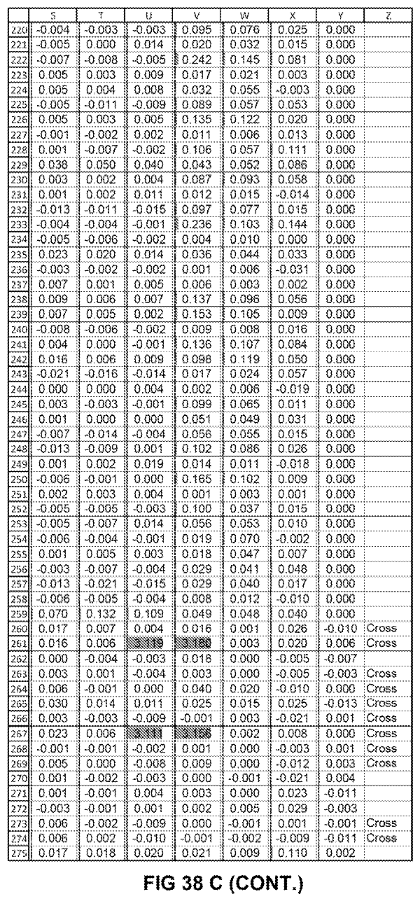

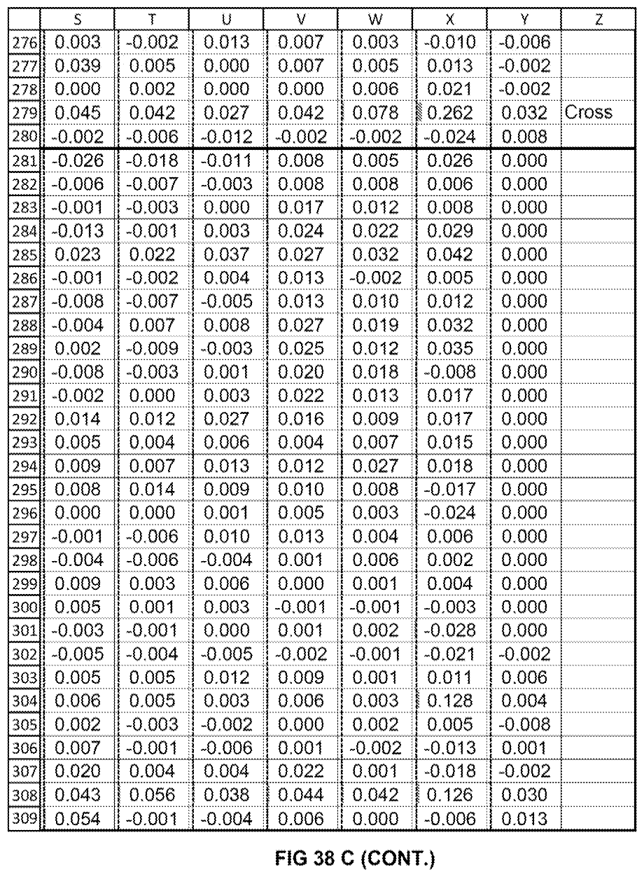

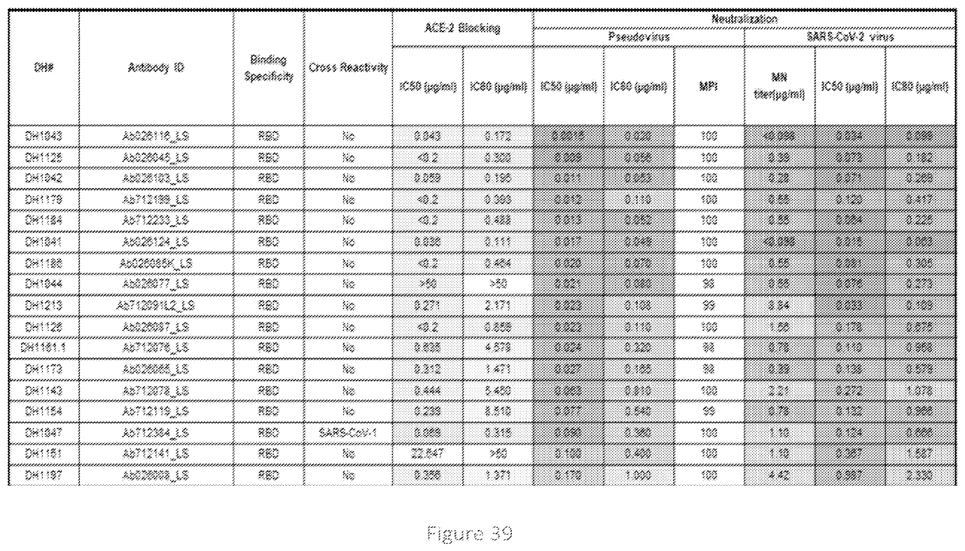

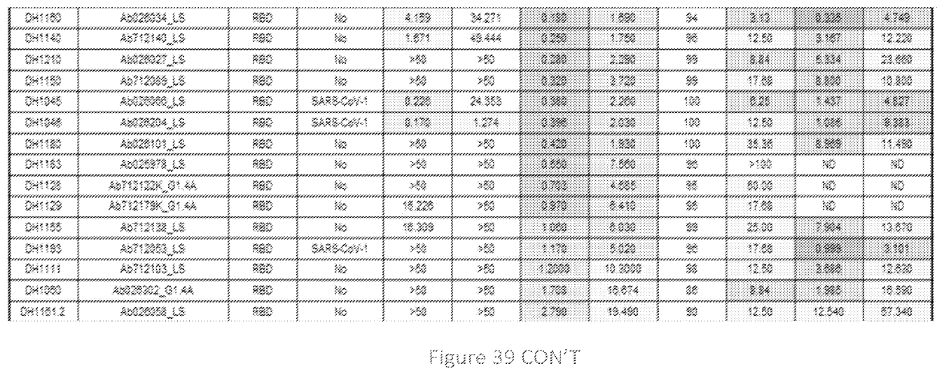

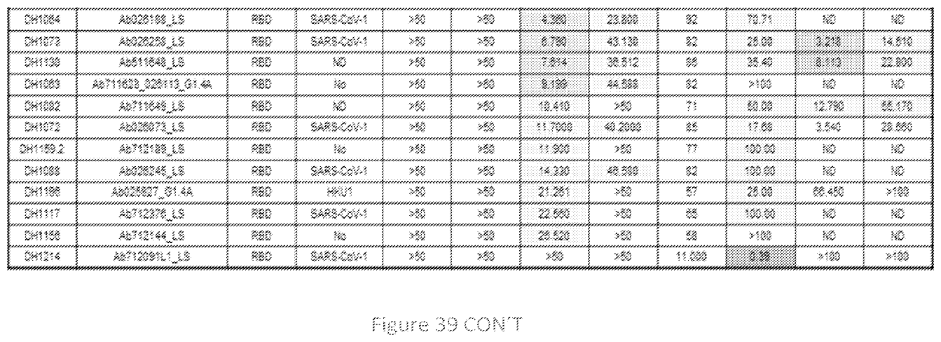

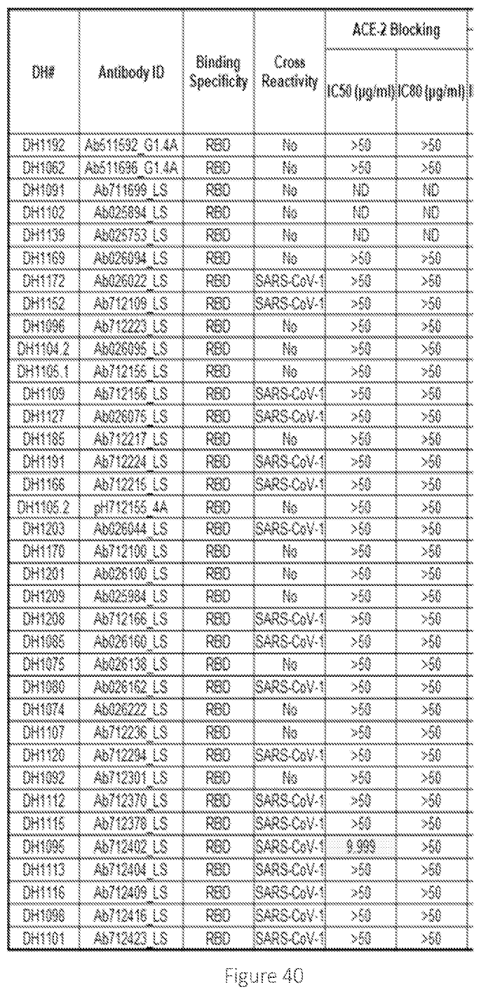

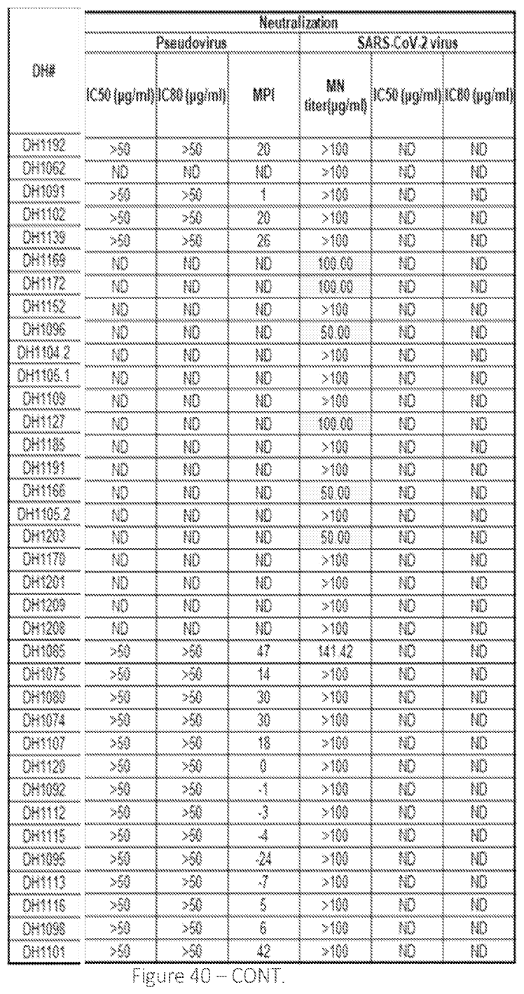

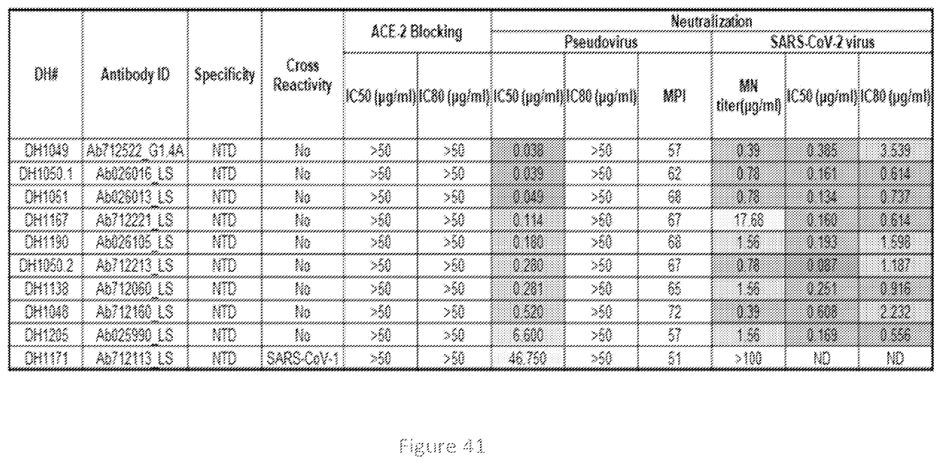

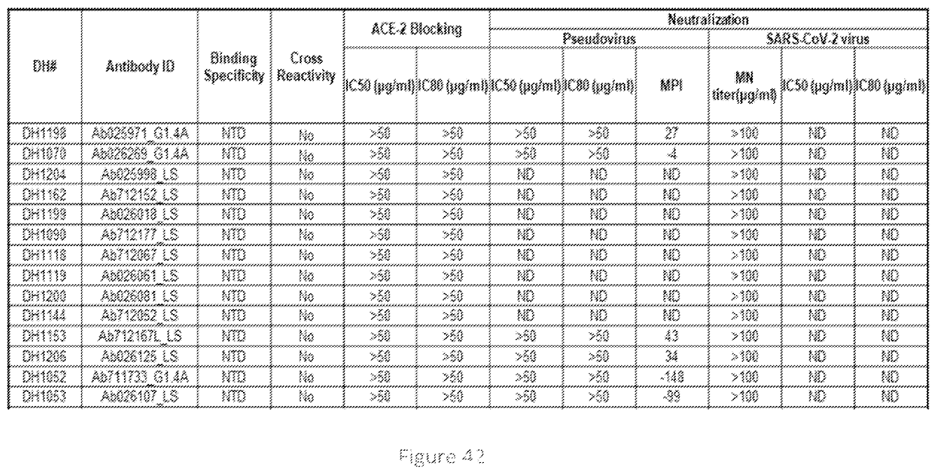

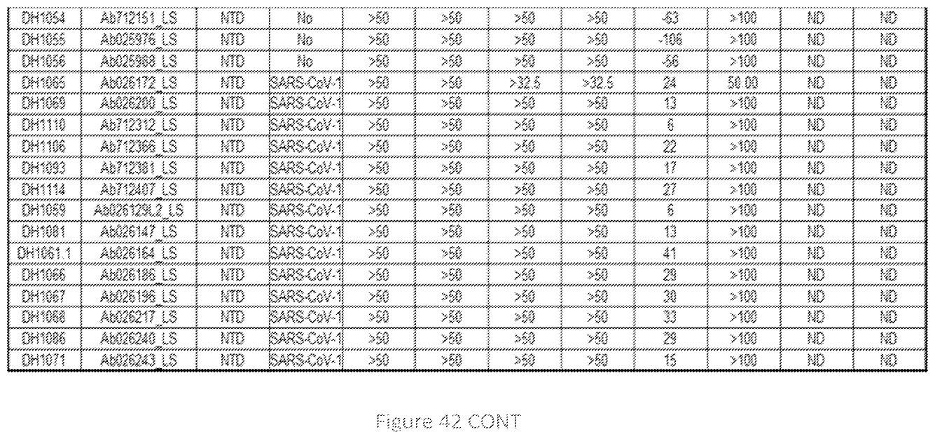

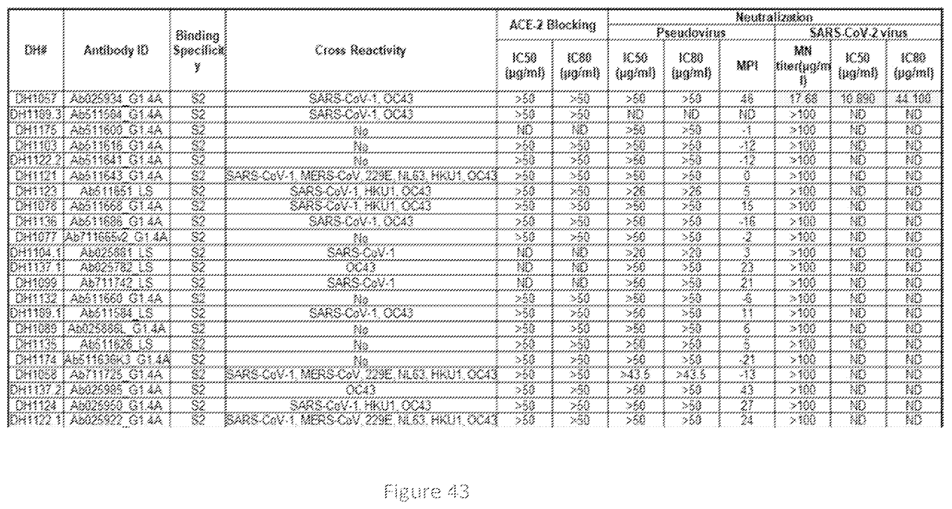

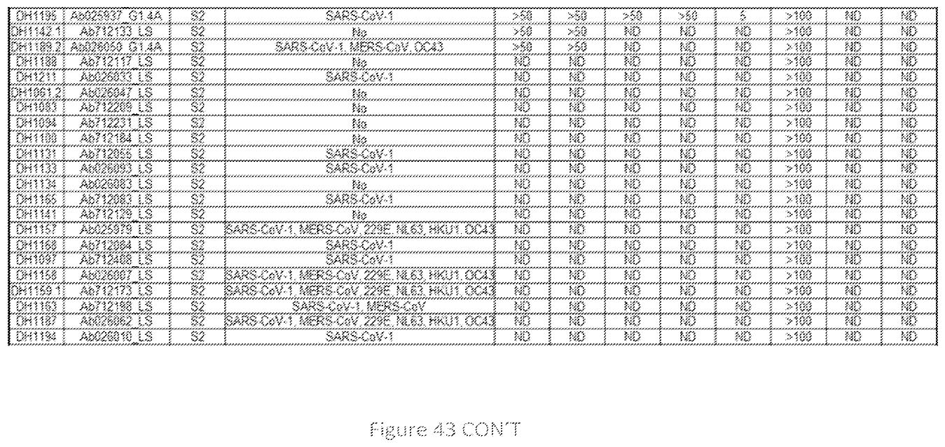

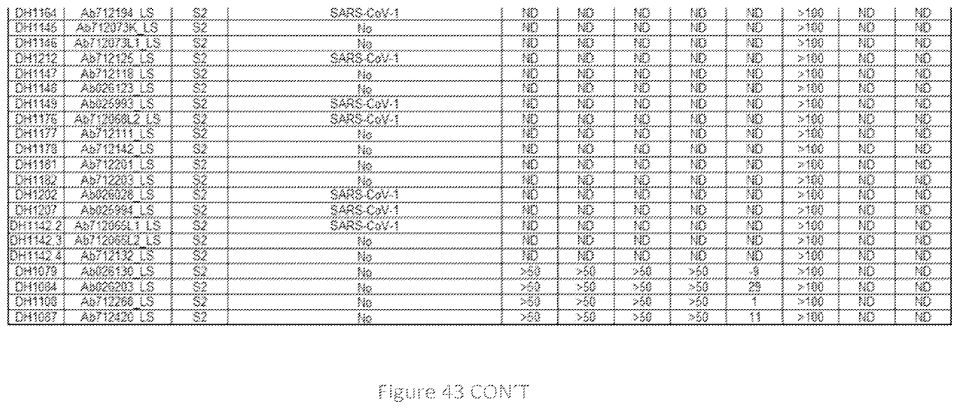

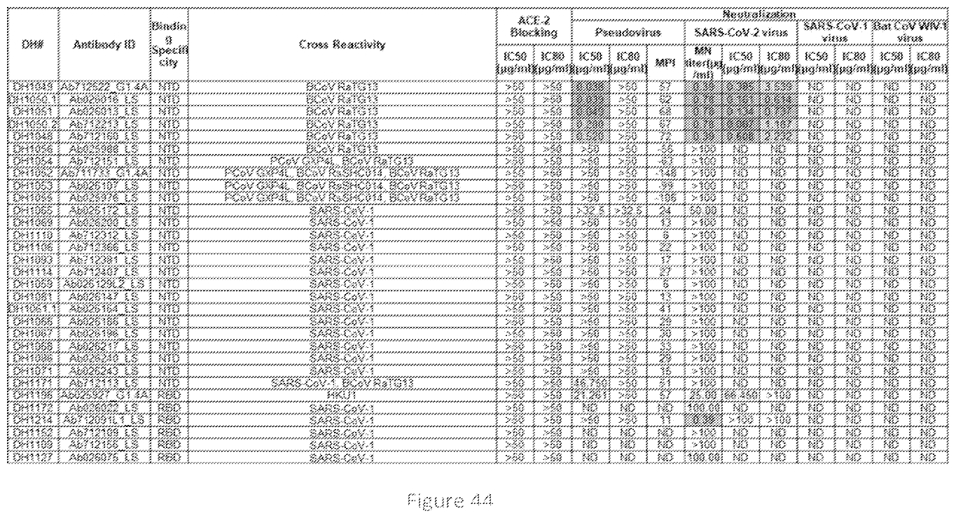

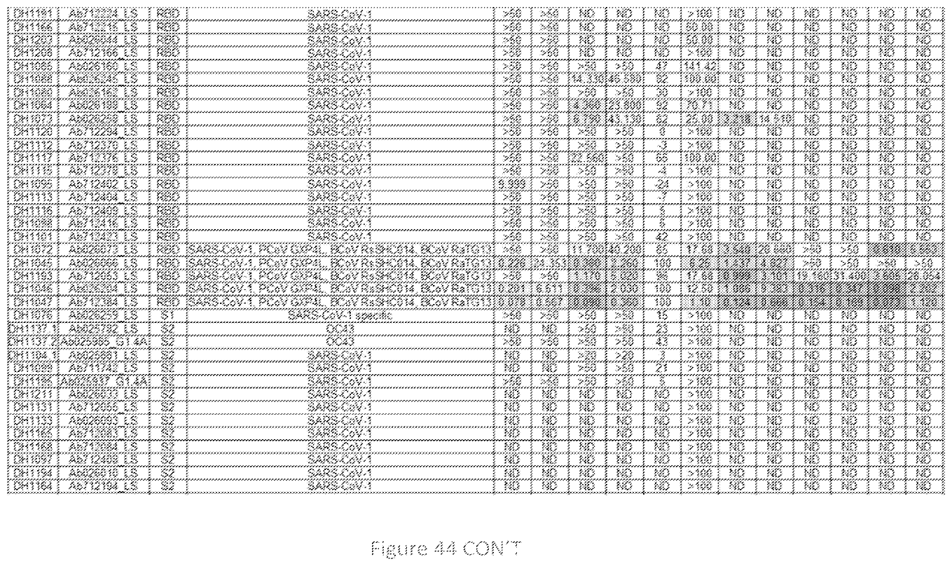

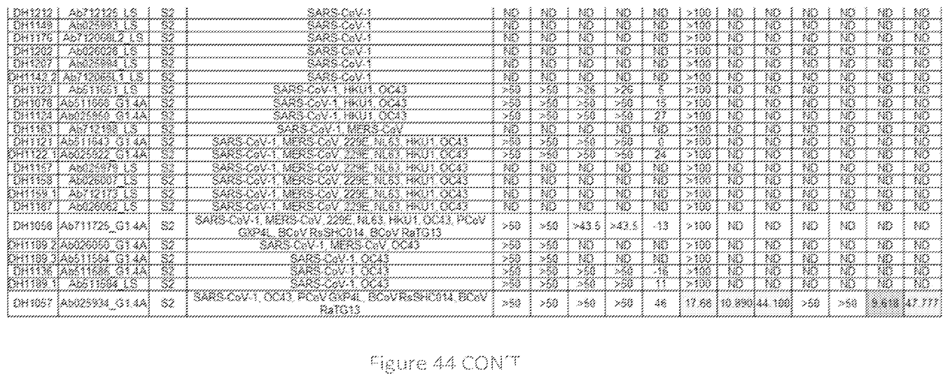

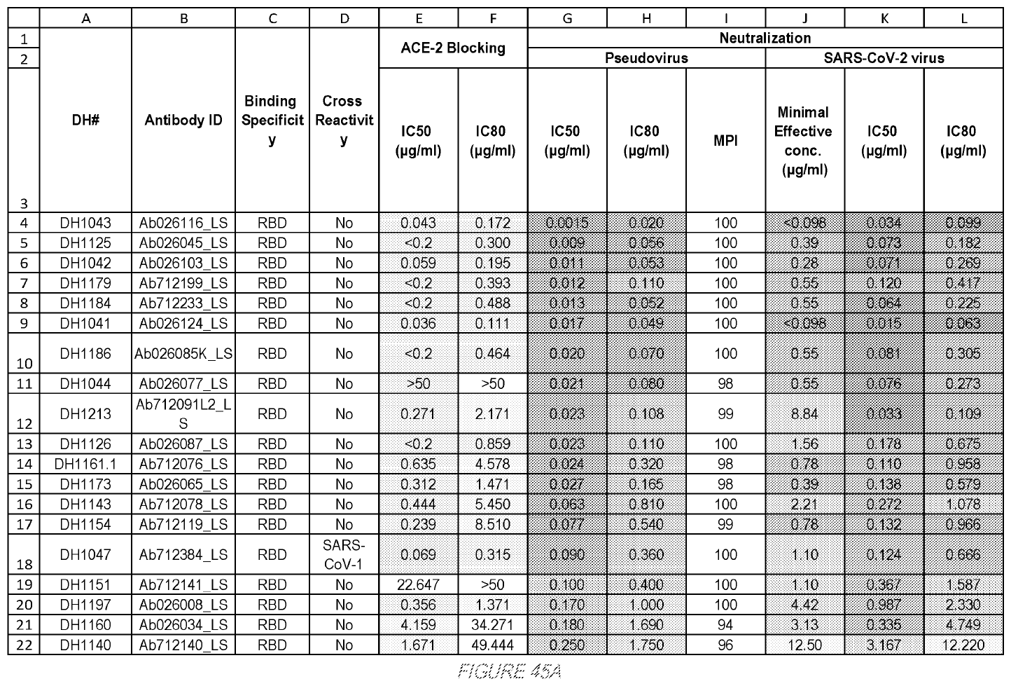

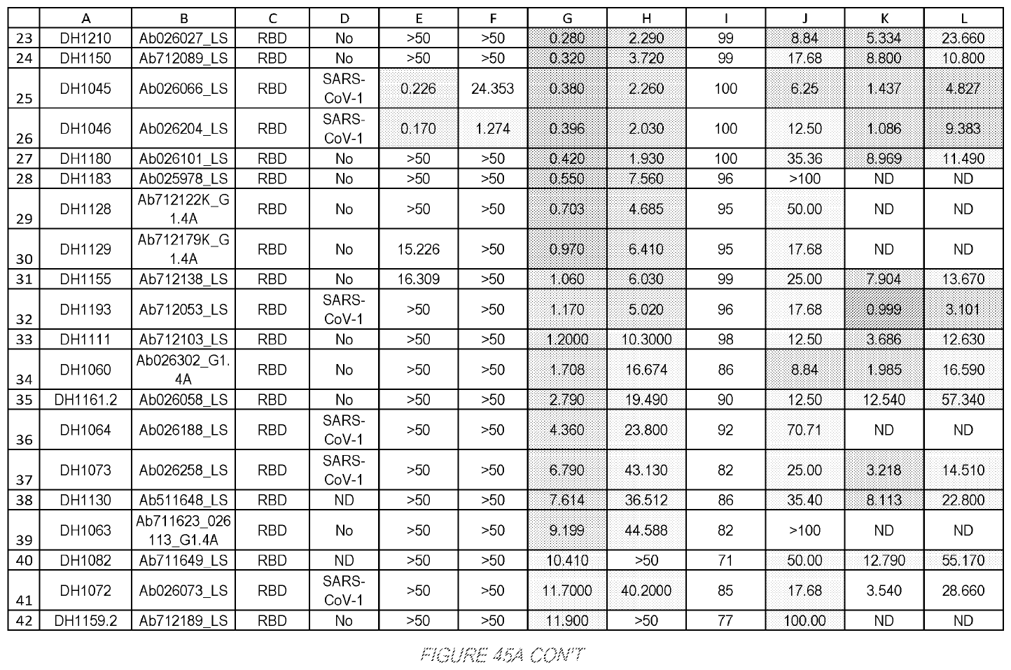

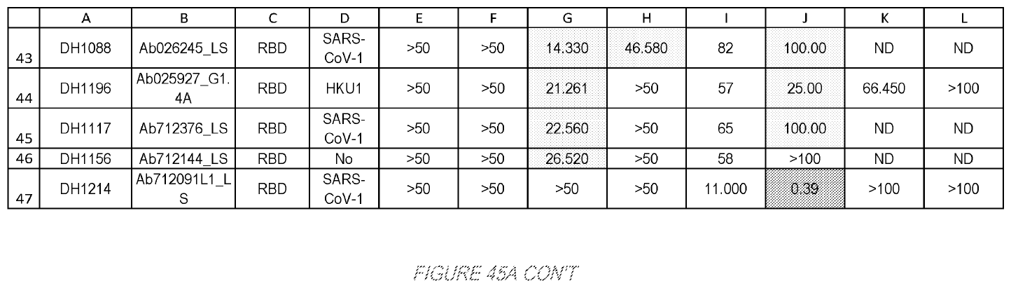

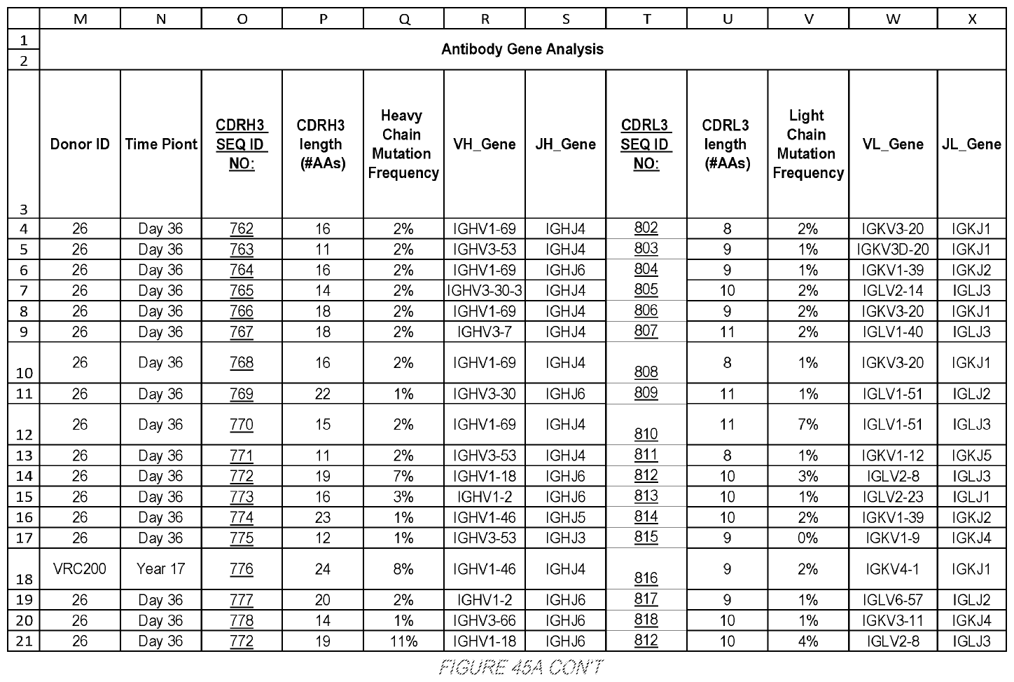

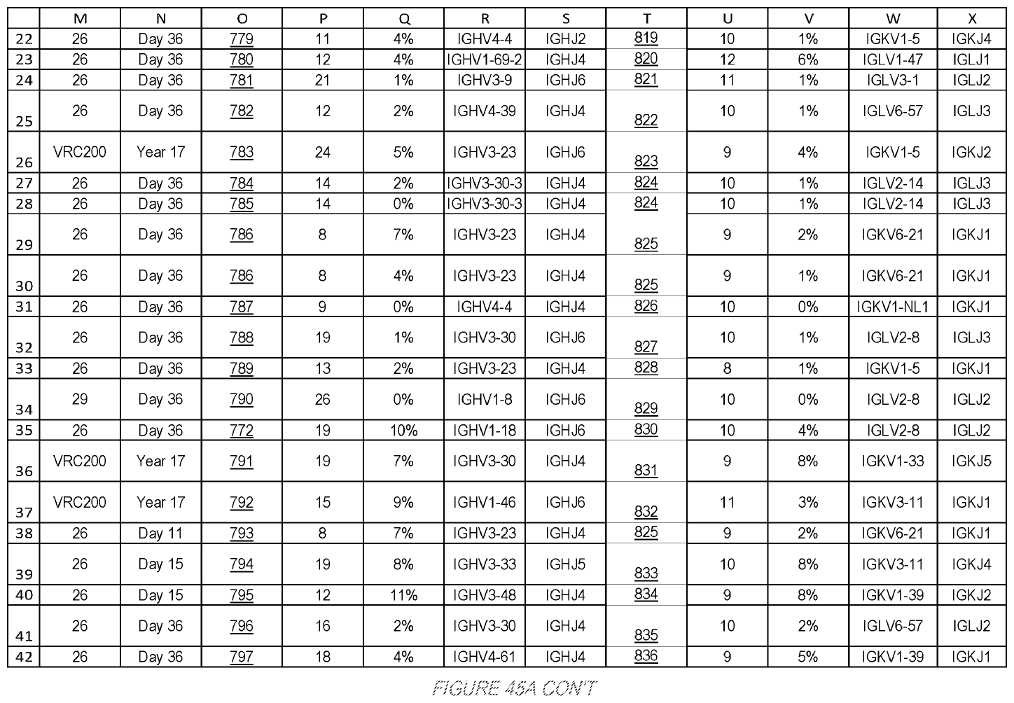

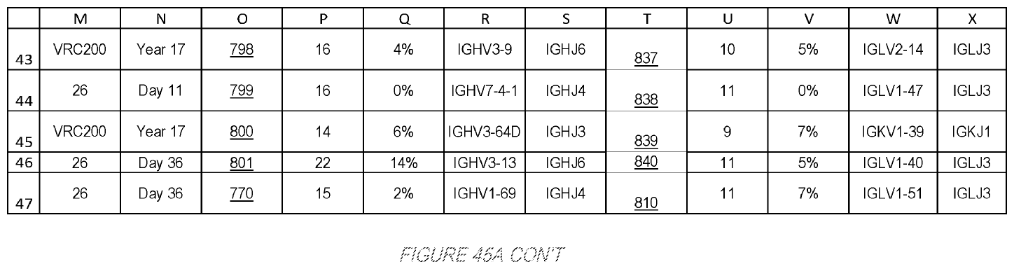

SARS-COV-2-reactive human monoclonal antibodies from plasmablasts or SARS-CoV-2-reactive memory B cells were isolated by flow cytometric sorting (40. 41) from a SARS-COV-2 infected individual 11. 15. 36, and 70 days post-onset of symptoms. Additional memory B cells were isolated from an individual infected with SARS-COV-1 ˜17 years prior to sample collection (FIGS. 1A-B, FIGS. 7 and 8). We isolated and characterized 1.737 antibodies, which bound to SARS-COV-2 S and nucleocapsid (NP) proteins (FIG. 1C, FIG. 38). We selected 187 antibodies for further characterization, and first examined neutralization of SARS-COV-2 pseudovirus and replication-competent SARS-COV-2. Forty-four of 81 recombinant RBD antibodies exhibited neutralization when assayed in 293T/ACE-2 cell pseudovirus. SARS-COV-2 microneutralization, or SARS-COV-2 plaque reduction assays (FIGS. 9A9, FIGS. 39-40).

Ten of the forty-one NTD antibodies neutralized in the 293T/ACE2 pseudovirus and plaque reduction assays, with the most potent antibody neutralizing pseudovirus with an IC50 of 39 ng/ml (FIGS. 9G-I, FIGS. 41-42). In addition. 5 non-neutralizing NTD antibodies enhanced SARS-COV-2 pseudovirus infection in 293T/ACE2 cells by 56% to 148% (FIG. 1D)). To determine whether this effect was applicable to replication-competent virus, we tested these five antibodies for enhancement of SARS-COV-2 live virus infection of Vero cells. Infection of replication-competent SARS-COV-2 nano-luciferase virus (42) also increased in the presence of each of the 5 NTD antibodies (FIG. 1E). Both ACE2-expressing 293T cells used for pseudovirus assays and Vero cells lack FcγR expression (43). Thus. NTD enhancement of SARS-COV-2 infection was FcγR-independent.

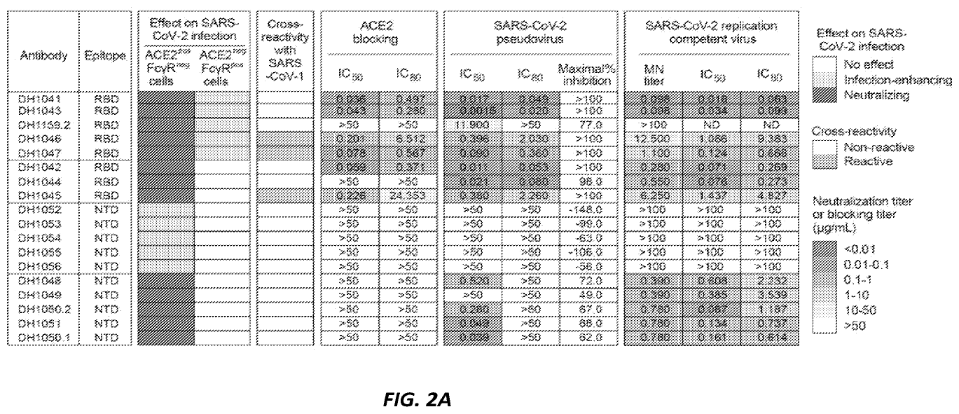

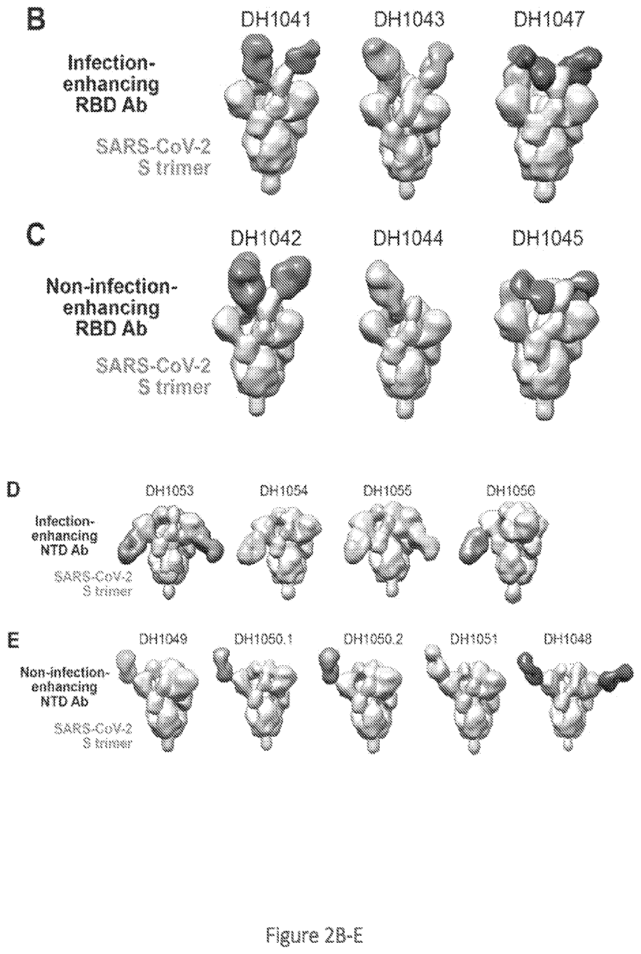

Previous studies have demonstrated FcγR-mediated ADE of SARS-COV-1 infection in ACE2-negative cells (31-37). Here. FcγR-dependent infection enhancement was determined by generating stable TZM-bl cell lines that expressed individual human FcγRs (FcγRI. FcγRIIa. FcγRIIb or FcγRIII). TZM-bl cells naturally lack ACE-2 and TMPRSS2 receptors, thus SARS-CoV-2 was unable to infect TZM-bl FcγR-expressing TZM-bl cells (FIG. 1F). One hundred S-reactive IgG1 antibodies selected from FIGS. 38-45 were tested for their ability to facilitate SARS-COV-2 infection of TZM-bl cells. Three of the antibodies enabled SARS-COV-2 infection of TZM-bl cells expressing FcγRI, and five antibodies mediated infection of FcγRIIb-expressing TZM-bl cells (FIGS. 1F-J). Fab fragments of these antibodies abrogated infection enhancement of TZM-bl cells expressing FcγRI or FcγRIIb (FIGS. 1K-L). FcγRI and FcγRIIb-dependent infection-enhancing antibodies were specific for the RBD of S protein, consistent with a recent finding by another group using COVID-19 patient sera or a recombinant antibody (44). Thus. RBD antibodies can be either neutralizing in the 293T/ACE2 cell line, infection-enhancing in the TZM-bl-FcγR-expressing cell lines, or both (FIG. 2A). NTD antibodies can either be neutralizing or infection-enhancing in the 293T/ACE2 cell line or Vero E6 cells (FIG. 2A). Therefore, the repertoire of SARS-COV-2 antibodies included potent neutralizing RBD and NTD antibodies. FcγR-dependent, infection-enhancing RBD antibodies, and FcγR-independent infection-enhancing NTD antibodies.

### Characterization of Infection-Enhancing Spike Recombinant Antibodies

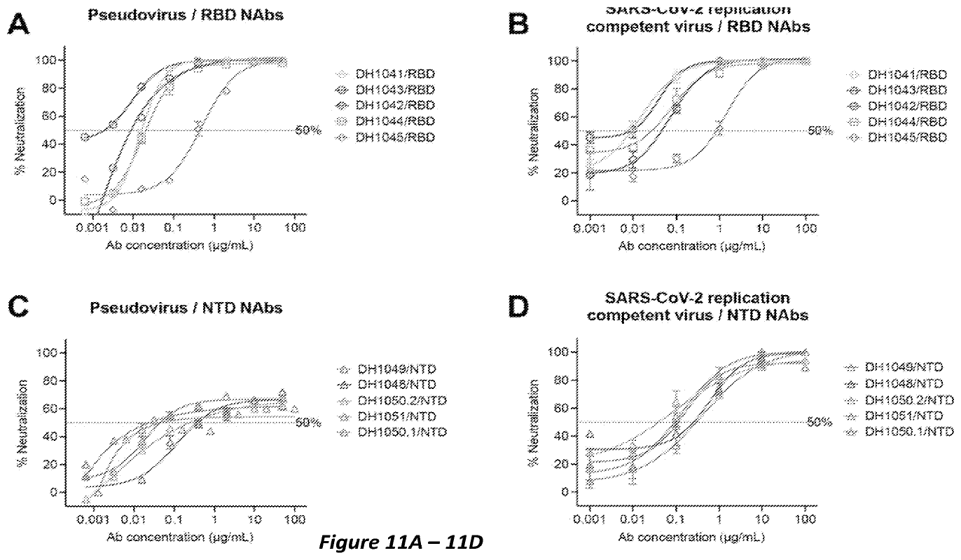

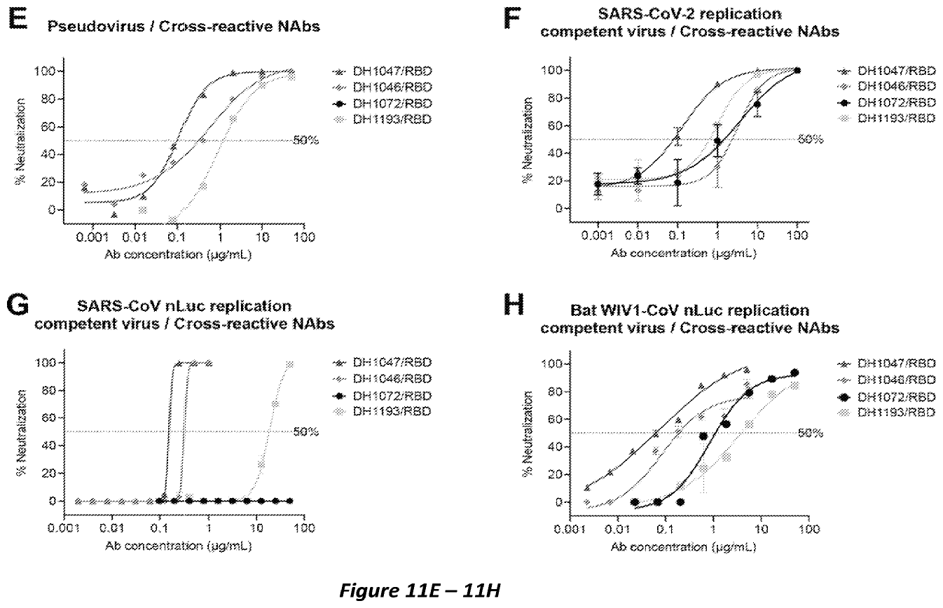

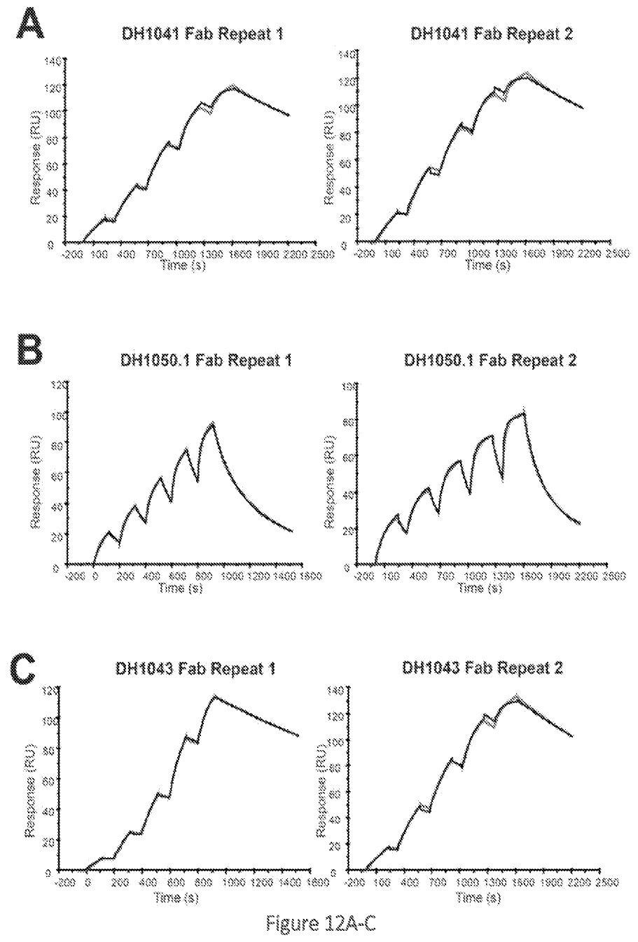

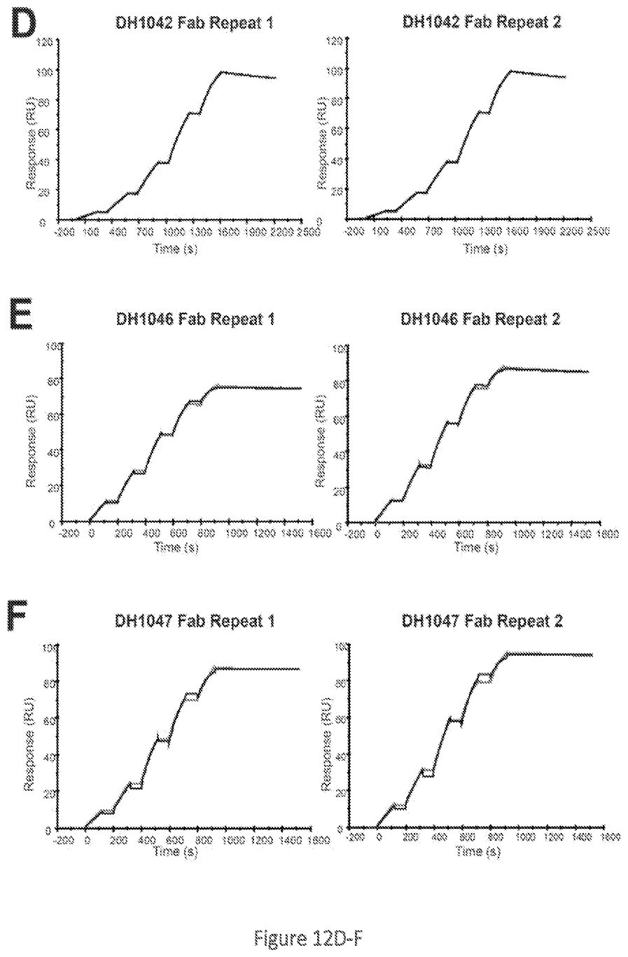

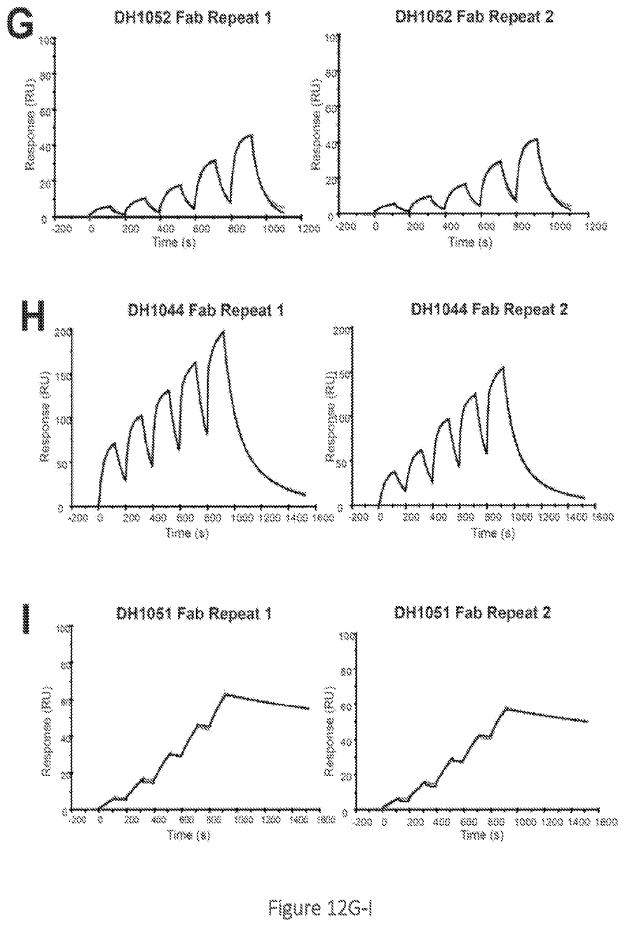

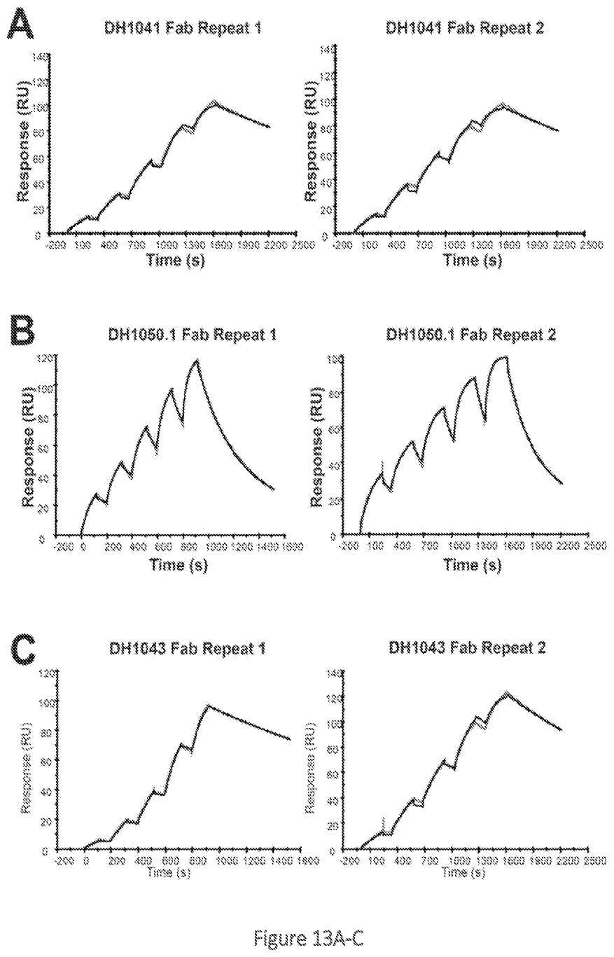

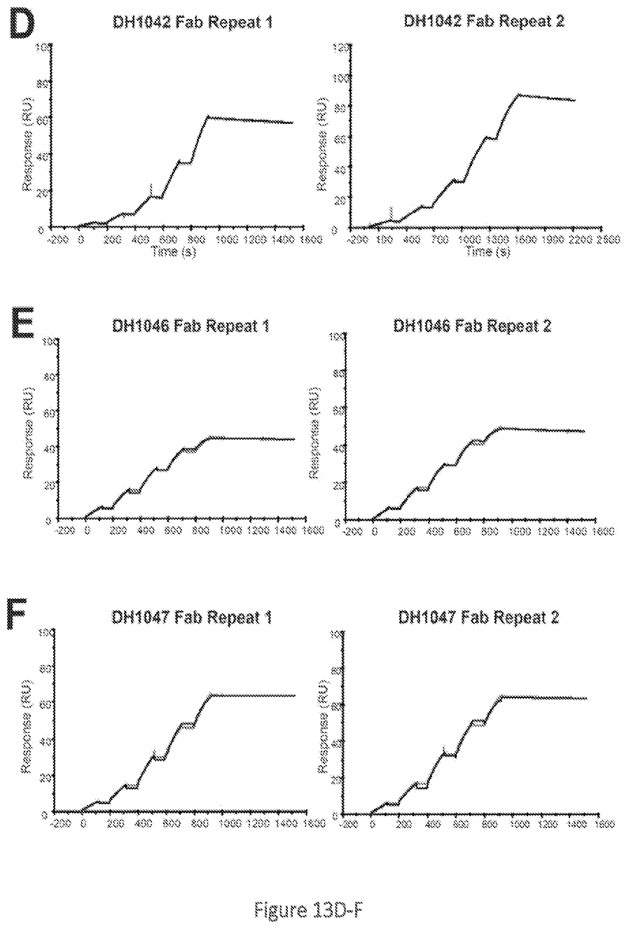

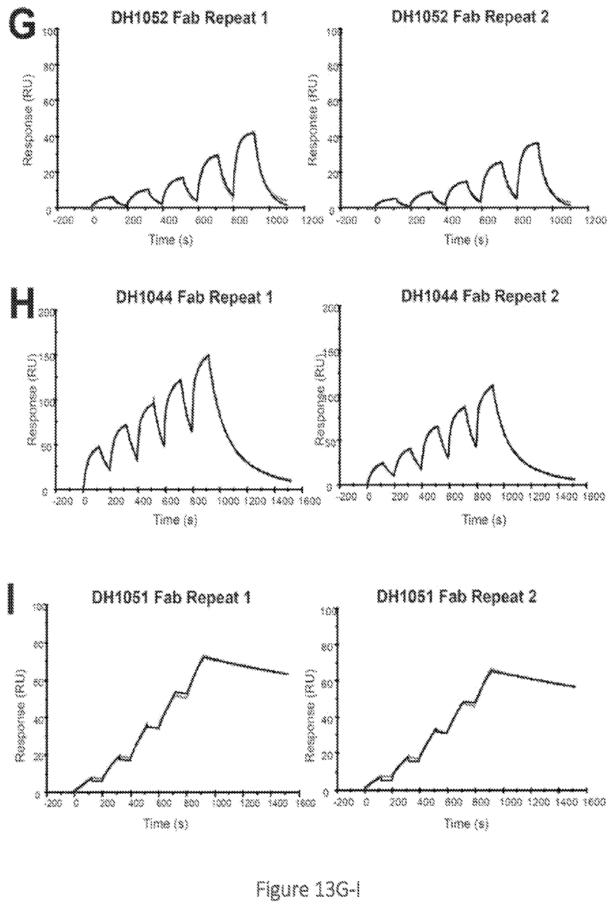

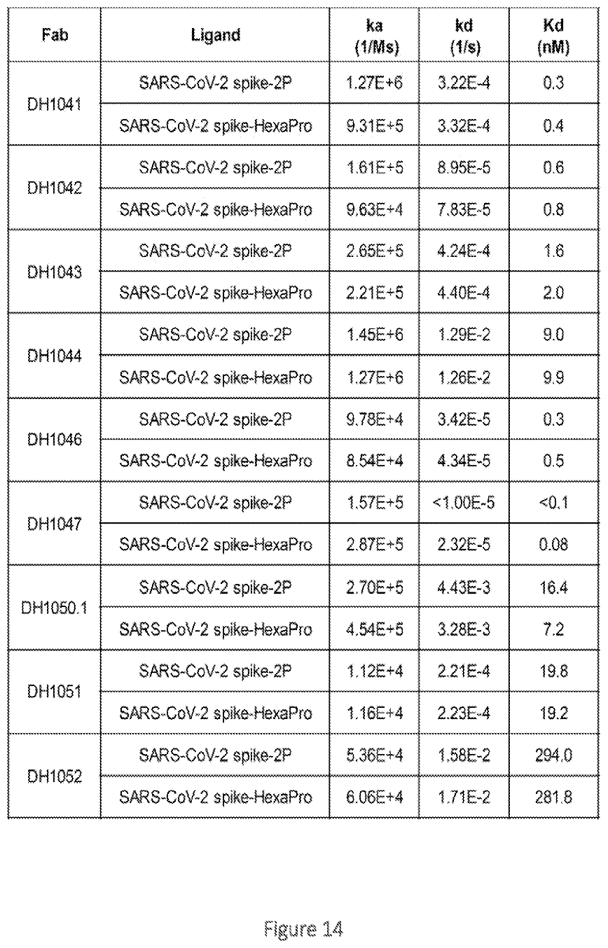

We compared the phenotypes and binding modes to S protein for five infection-enhancing RBD antibodies and three RBD antibodies that lacked infection enhancement to elucidate differences between these two types of antibodies. All selected RBD antibodies neutralized SARS-COV-2 pseudovirus and/or replication-competent virus in ACE2-expressing cells (FIG. 2A, FIG. 11), despite five of these antibodies mediating infection enhancement in ACE2-negative. FcγR-positive TZM-bl cells (FIGS. 1F-L and 2A,FIG. 11). Both types of selected RBD antibodies blocked ACE2 binding to S protein. Antibody antigen binding fragments (Fabs) of four of the infection-enhancing RBD antibodies and two of the non-infection-enhancing RBD antibodies bound to S with high affinities ranging from 0.1 to 9 nM (FIGS. 12-14). Thus, the infection-enhancing or non-enhancing RBD antibodies showed similarities in ACE2 blocking, affinity, and neutralization of ACE2-dependent SARS-COV-2 infection (FIG. 2A).

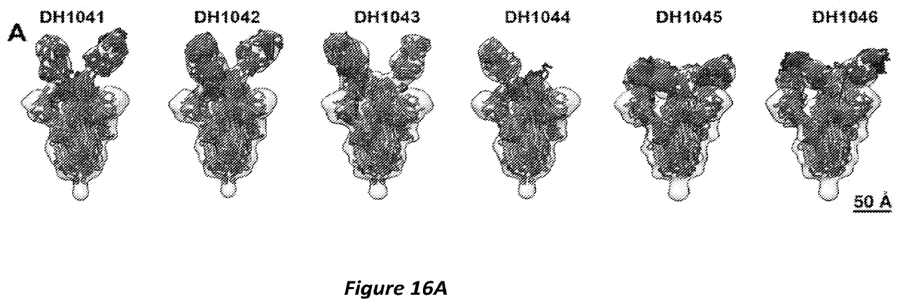

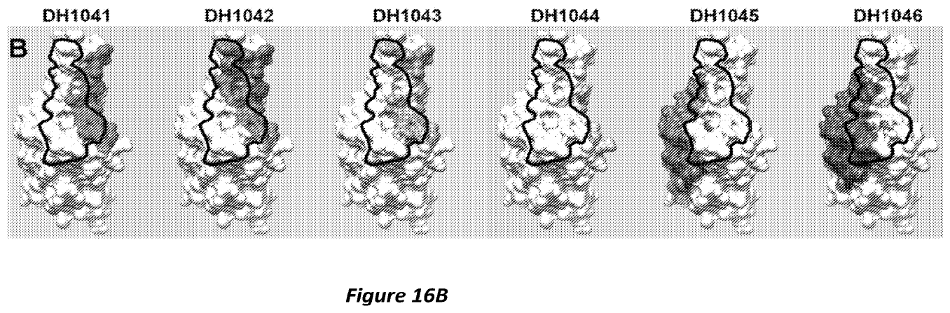

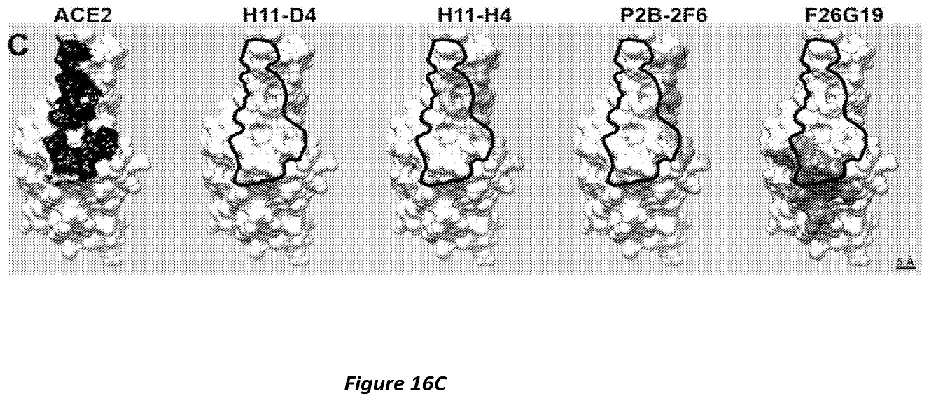

We obtained NSEM reconstructions of Fabs in complex with stabilized S ectodomain trimer for six of the eight representative RBD antibodies (45). Infection-enhancing RBD antibodies DH1041 and DH1043 bound with a vertical approach (FIG. 2B), parallel to the central axis of the S trimer, similar to non-infection-enhancing antibodies DH1042 and DH1044 (FIG. 2C). The epitopes of antibodies DH1041. DH1042, and DH1043 overlapped with that of the ACE-2 receptor (46), consistent with their ability to block ACE-2 binding to S protein (FIG. 2A and FIG. 16). Their epitopes were similar to those of three recently described antibodies. P2B-2F6 (47). H11-H4, and H11-D4 (FIG. 16) (48. 49). The epitope of another non-infection-enhancing RBD antibody DH1044 was only slightly shifted relative to DH1041.

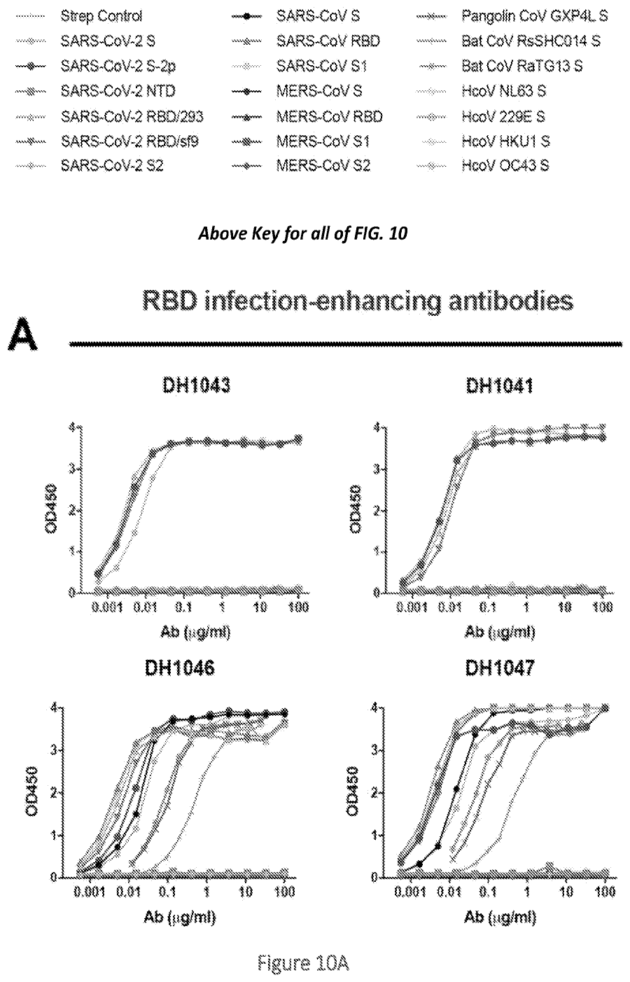

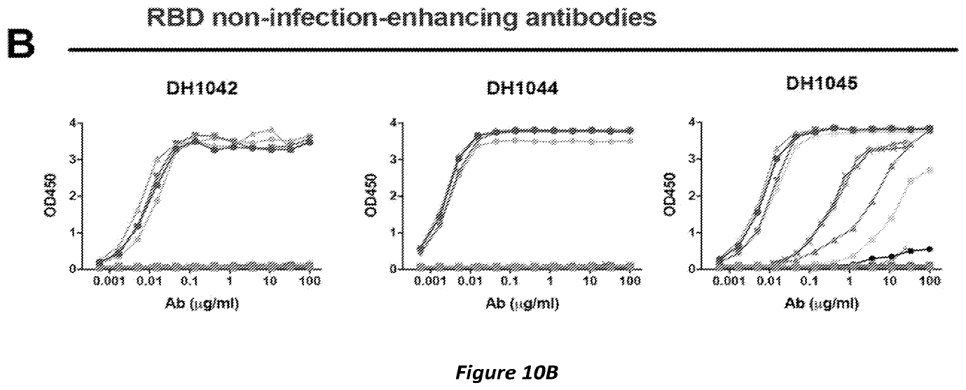

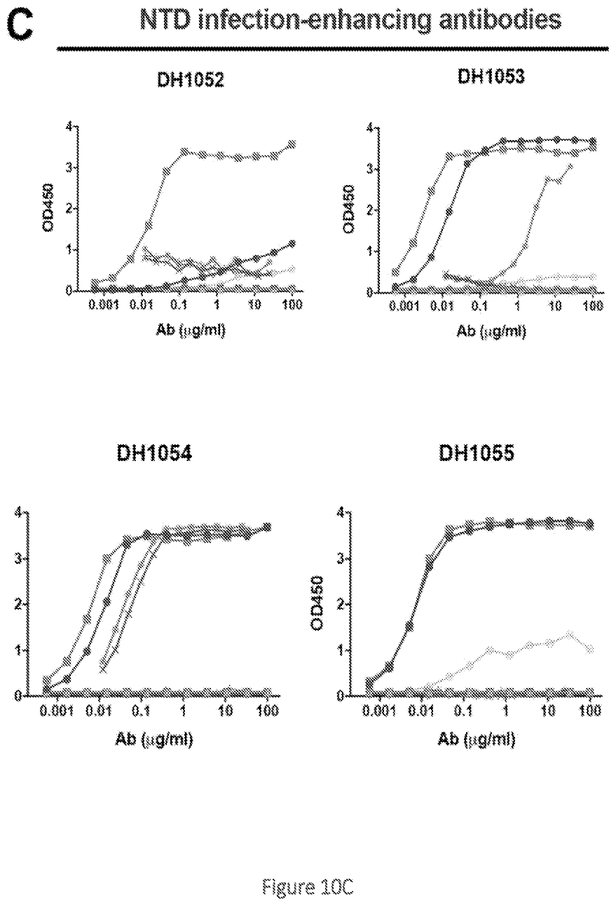

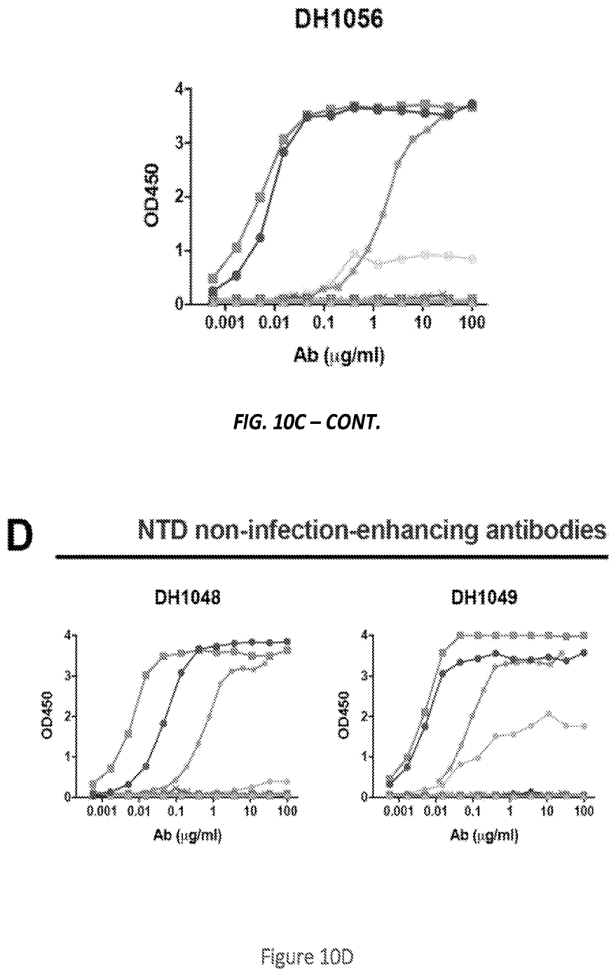

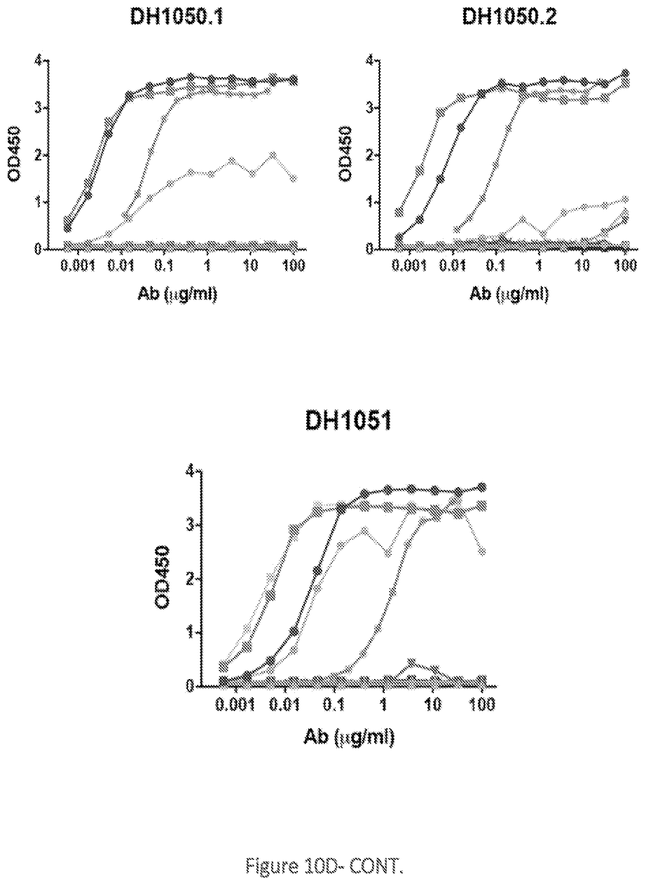

DH1042 and DH1043 (FIG. 2C), but resulted in DH1044 not blocking ACE2 binding (FIG. 2A and FIG. 16). The remaining two RBD antibodies, DH1045 and DH1047 cross-reacted with both SARS-COV-1 and SARS-COV-2 S (FIG. 2A and FIG. 10). Although DH1047 mediated FcγR-dependent infection of TZM-bl cells and DH1045 did not, both antibodies bound to RBD-up S conformations with a more horizontal angle of approach (FIG. 2B-C and FIG. 16) (50). Thus, epitopes and binding angles of RBD antibodies determined by NSEM did not discriminate between antibodies that mediated FcγR-dependent infection enhancement and those that did not.

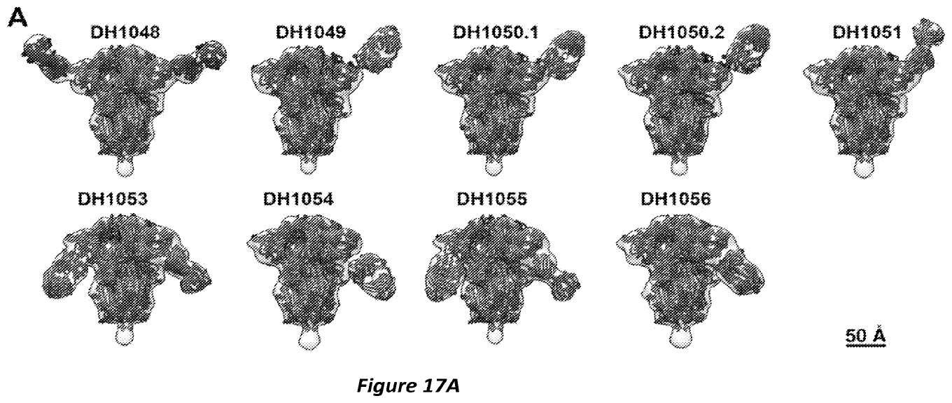

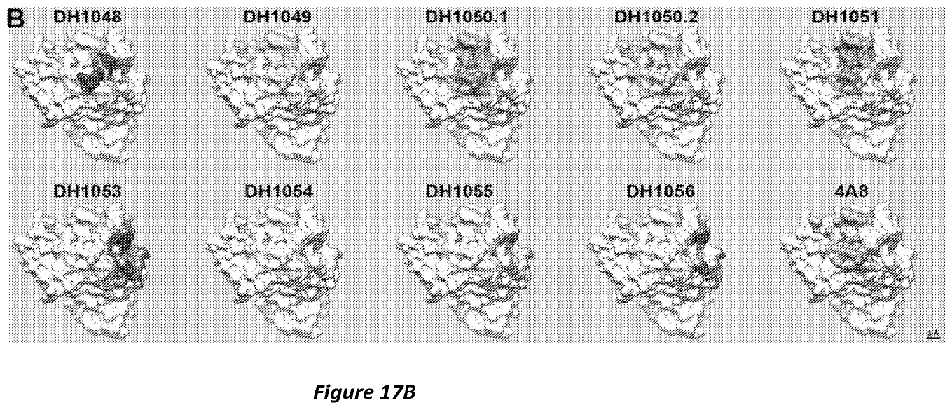

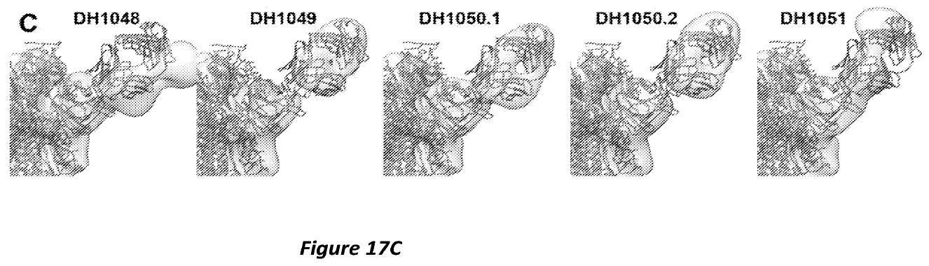

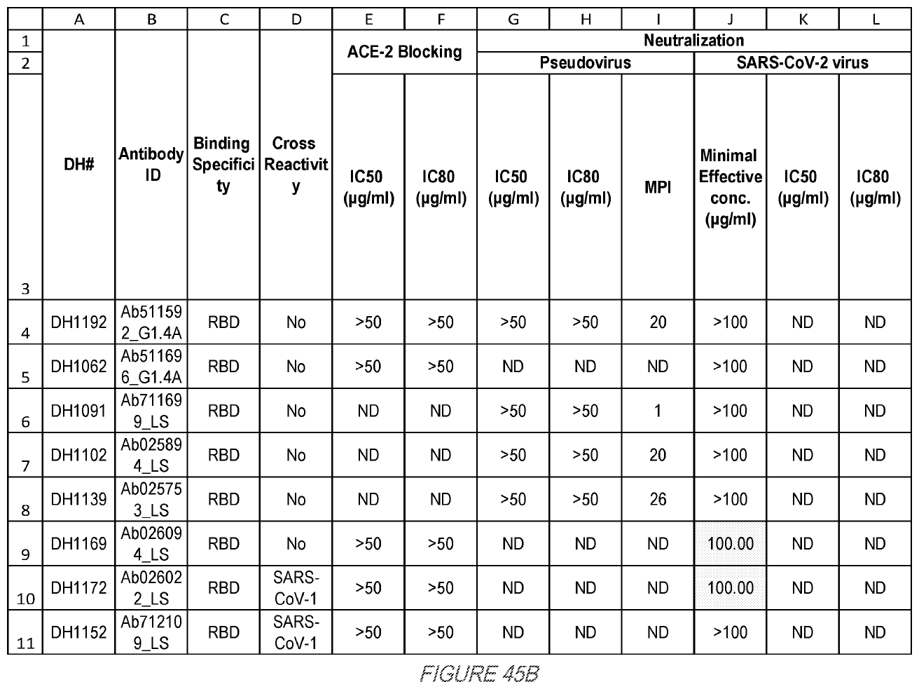

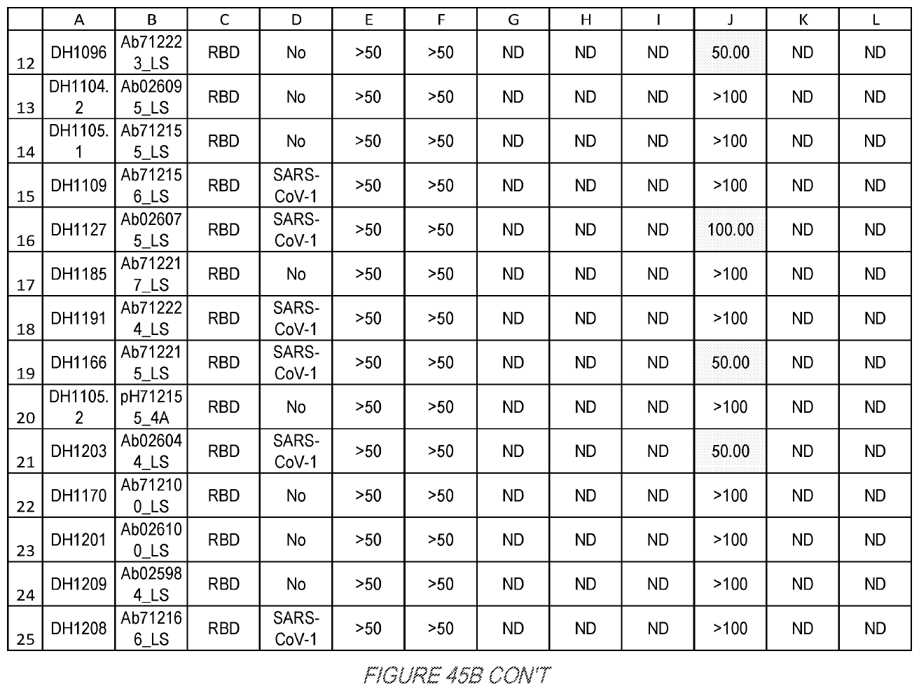

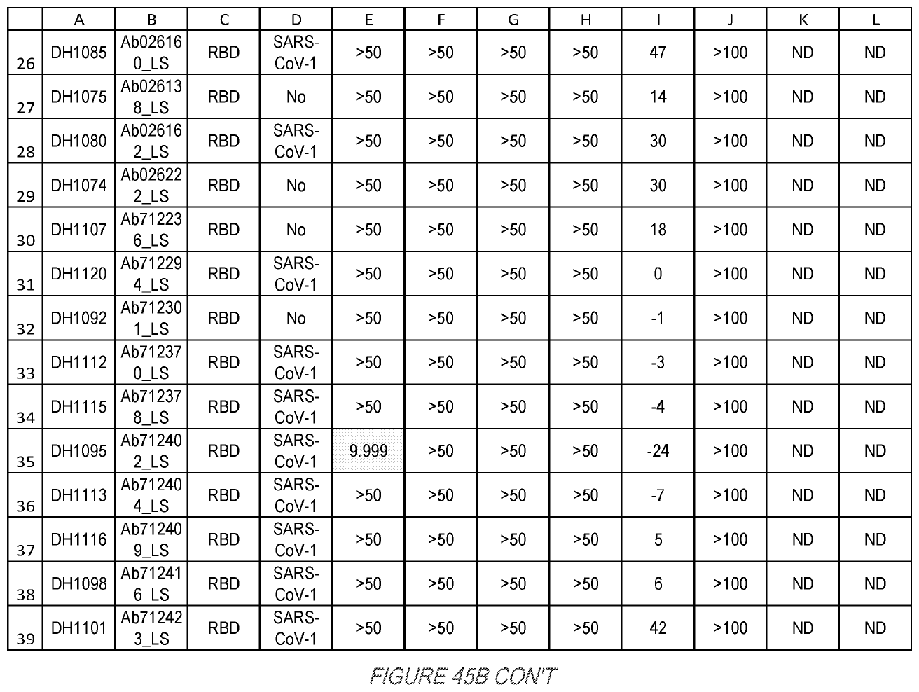

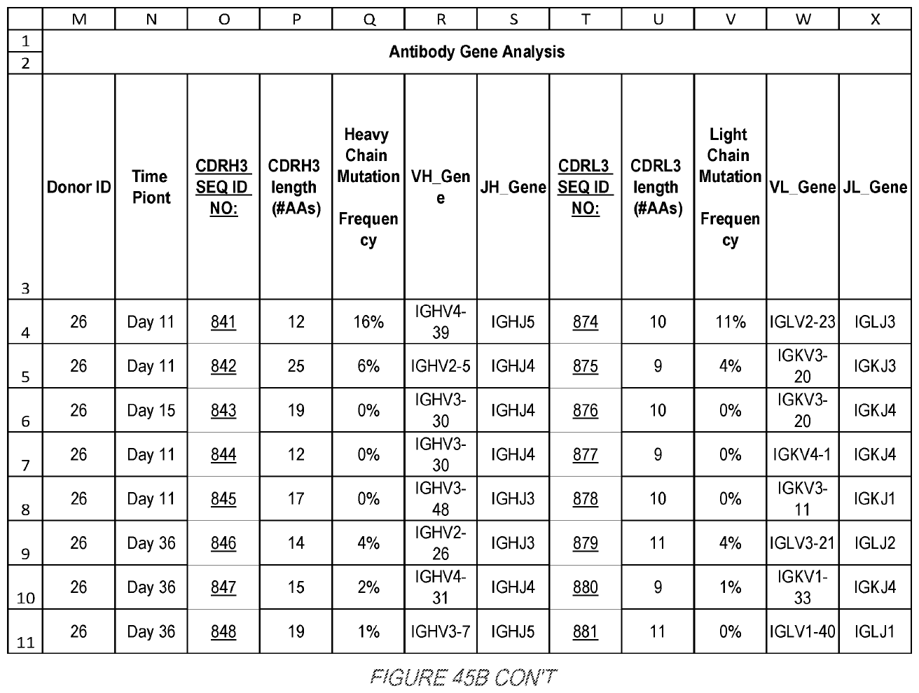

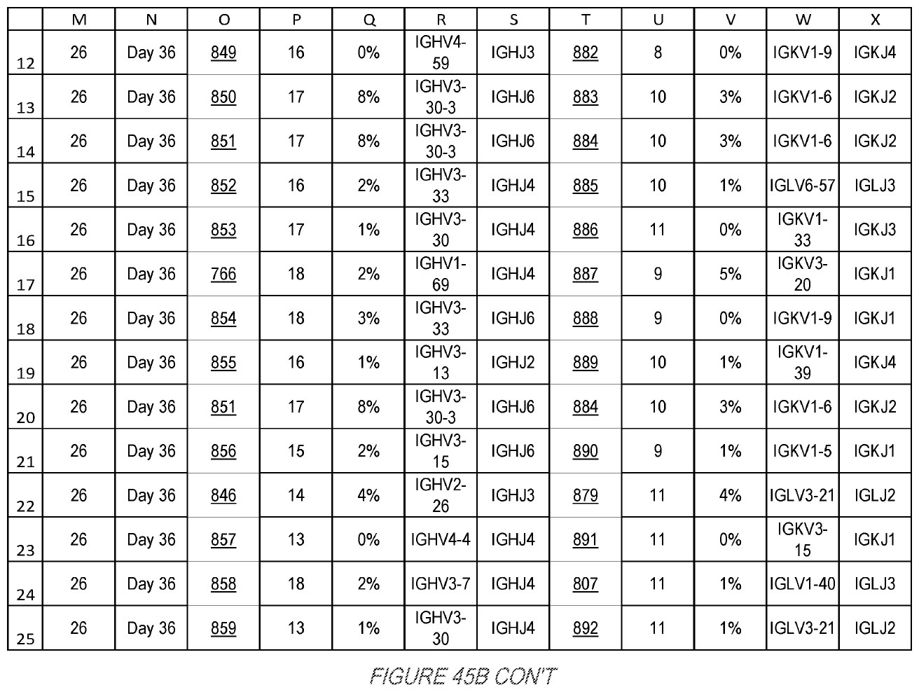

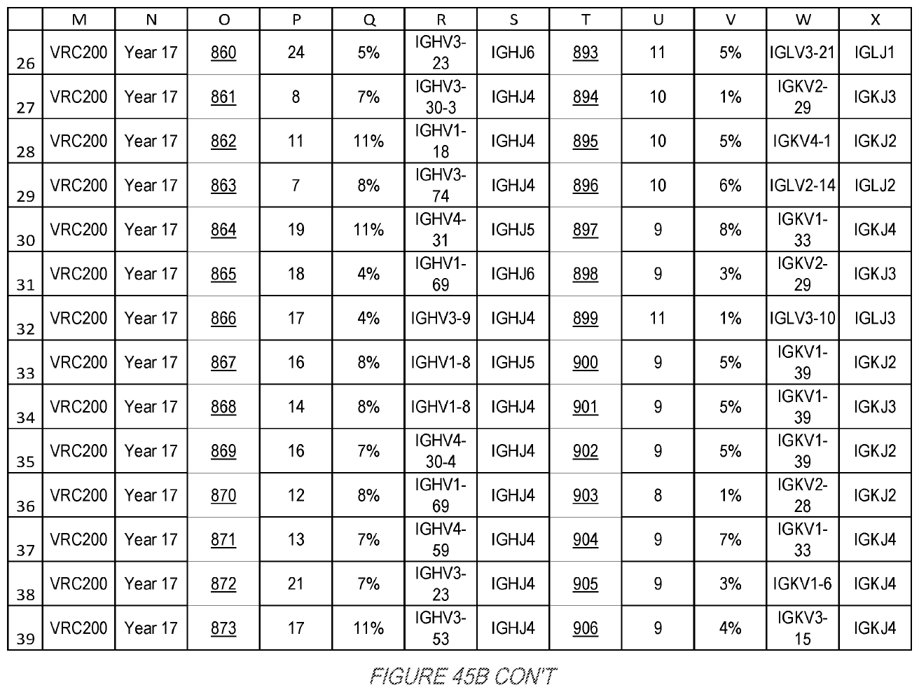

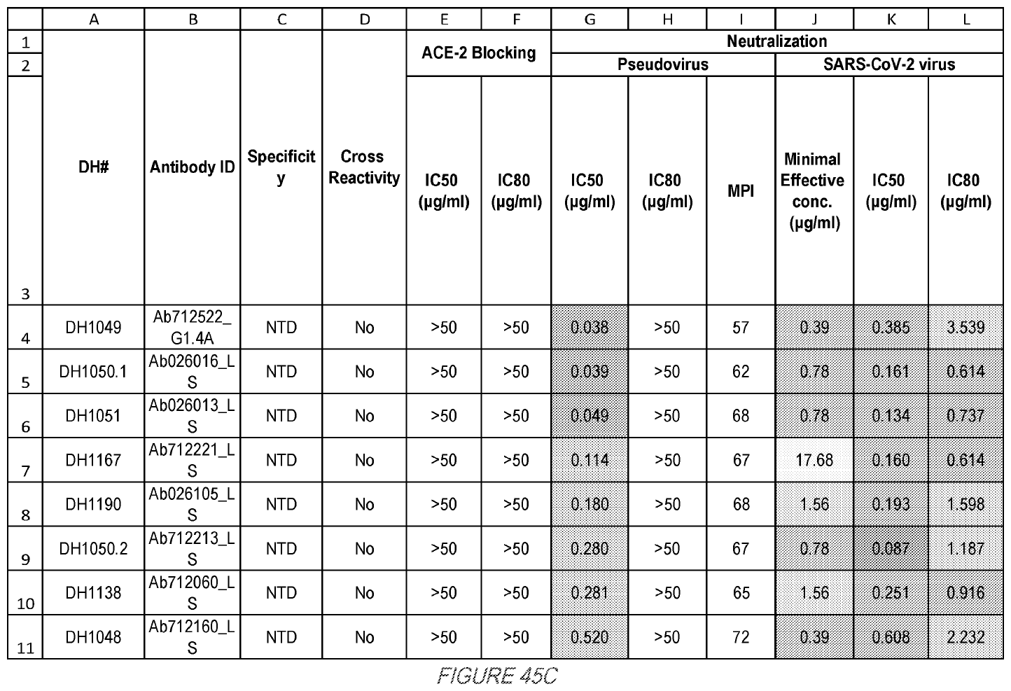

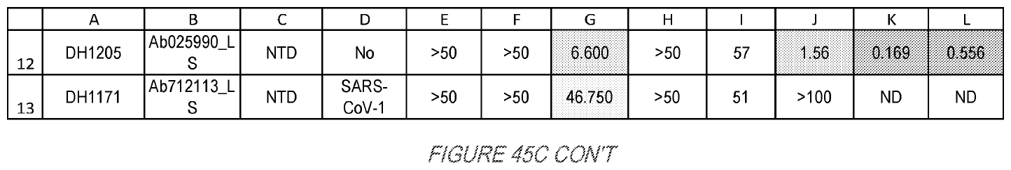

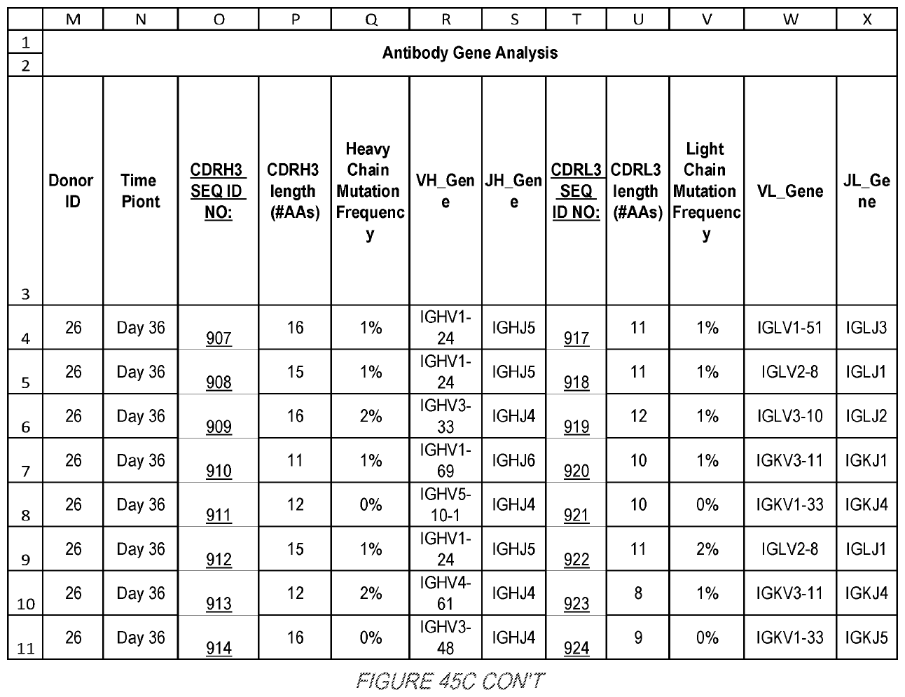

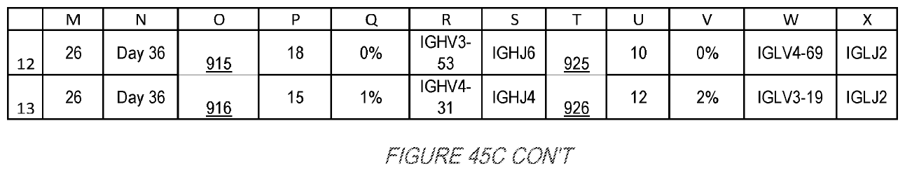

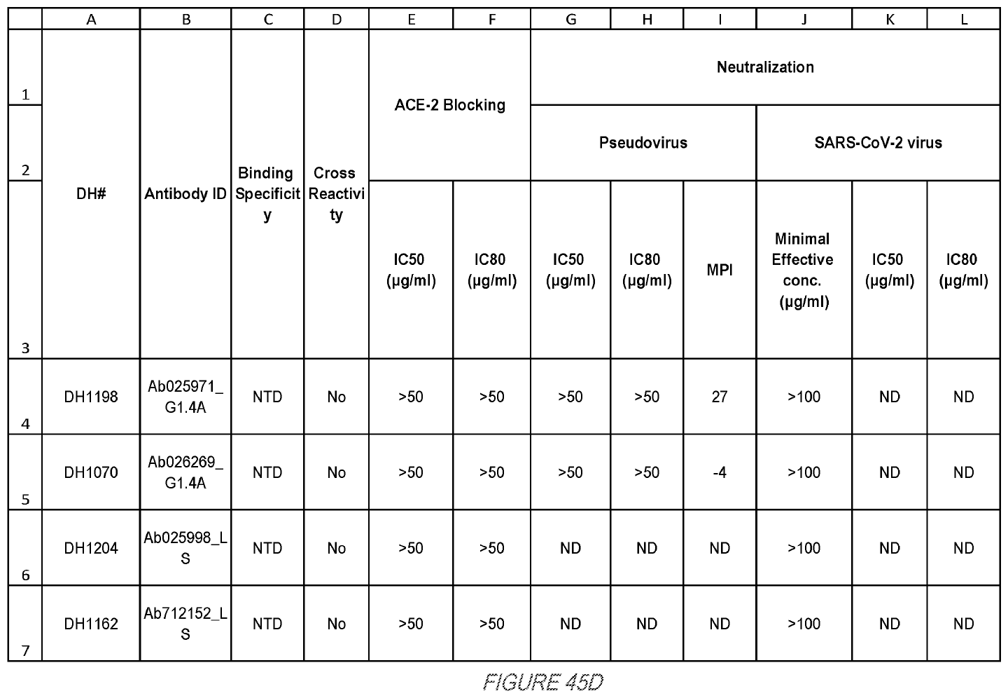

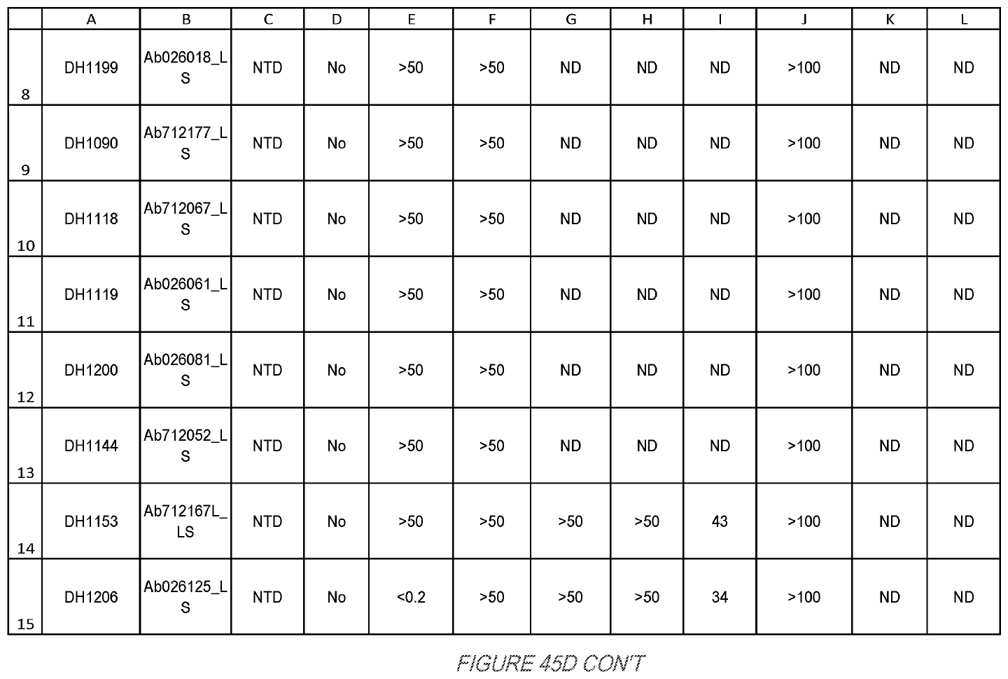

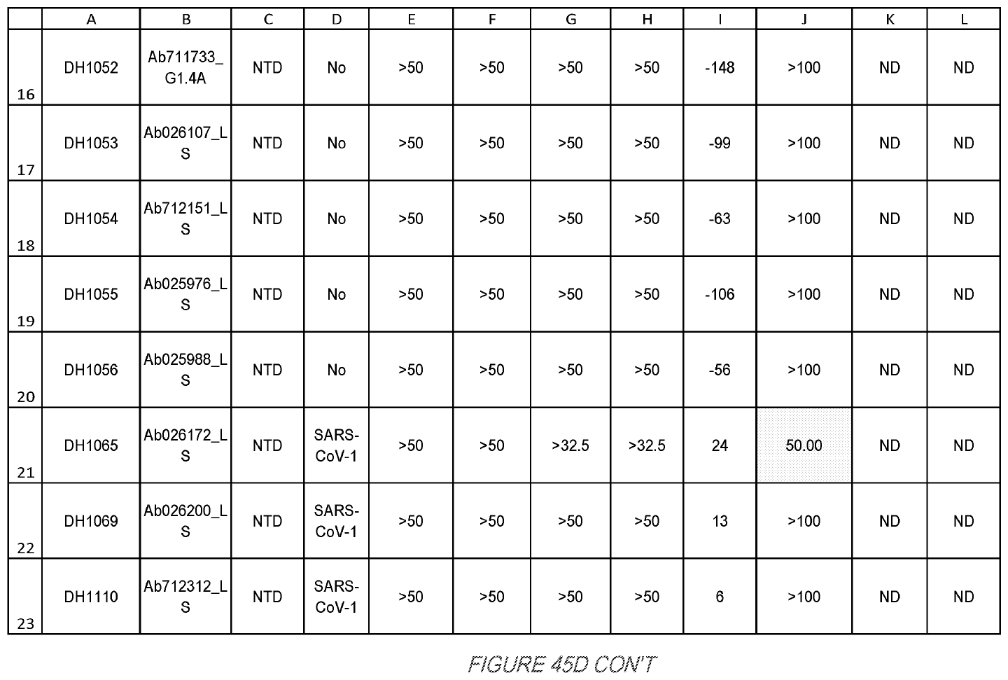

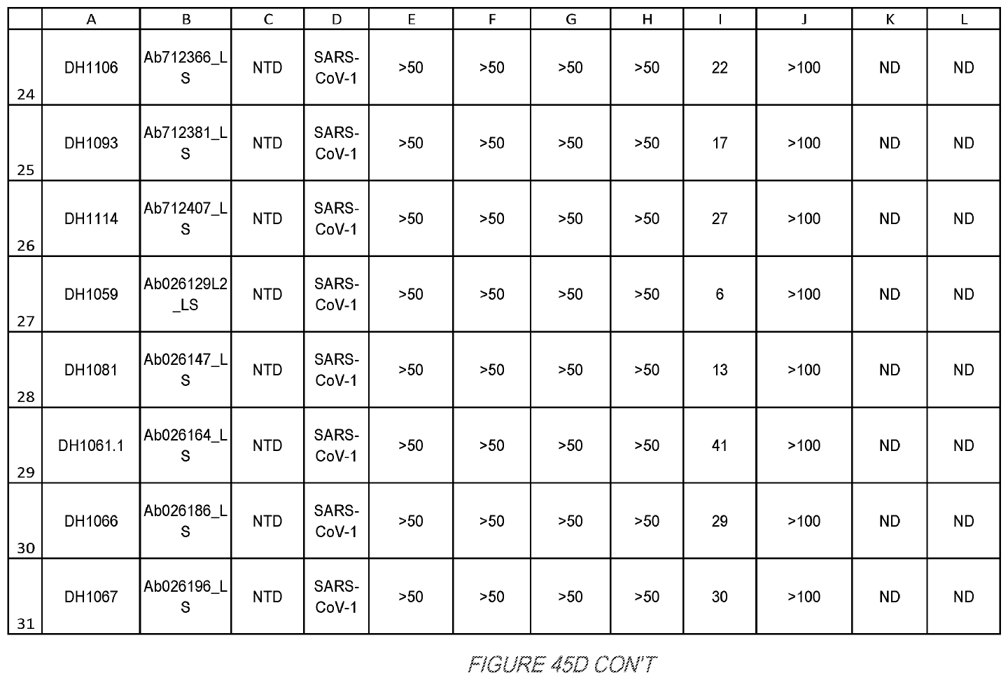

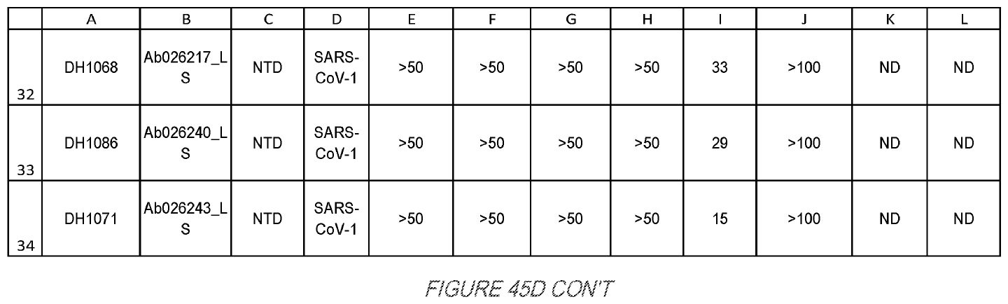

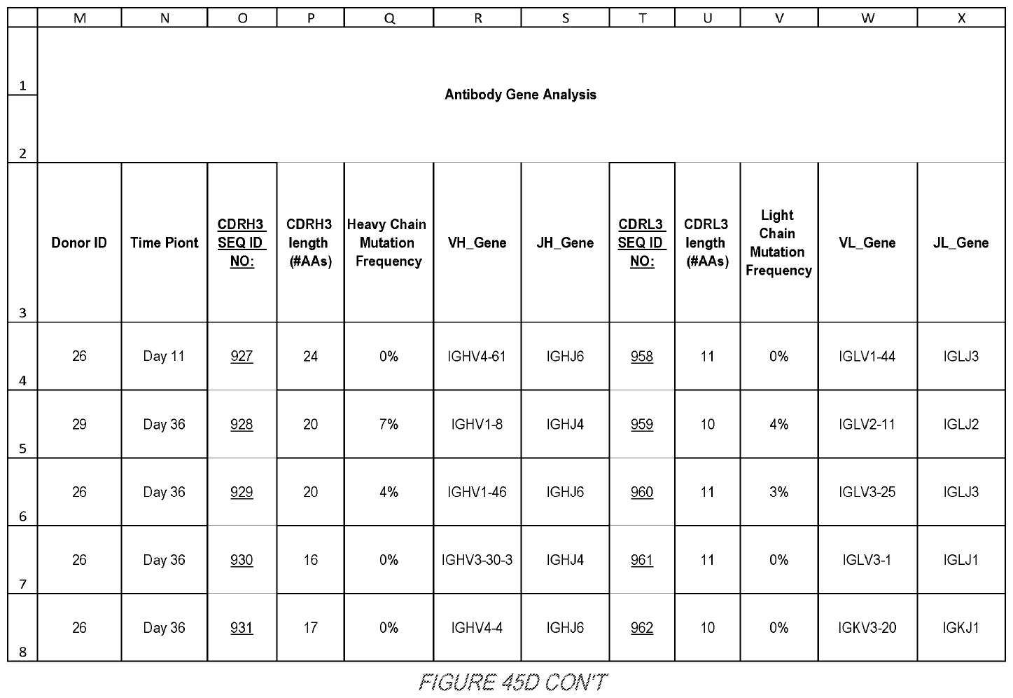

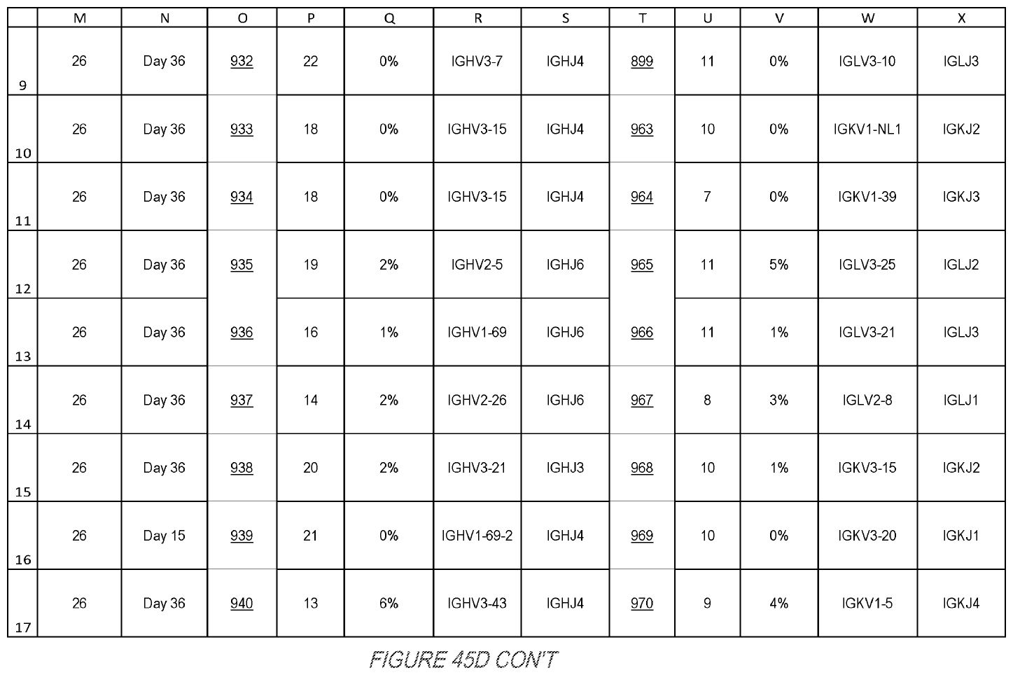

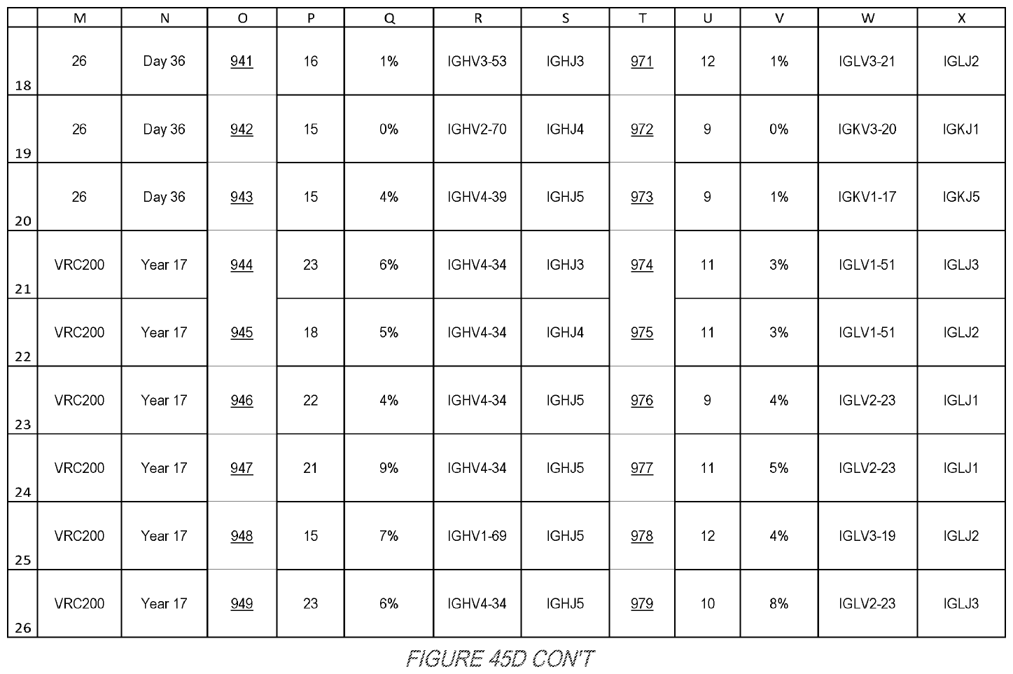

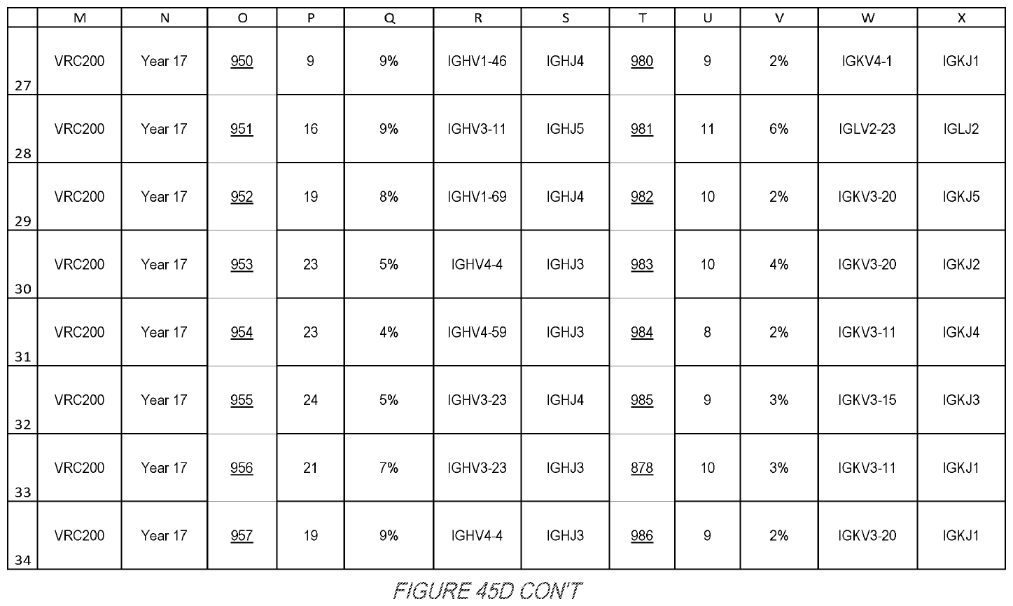

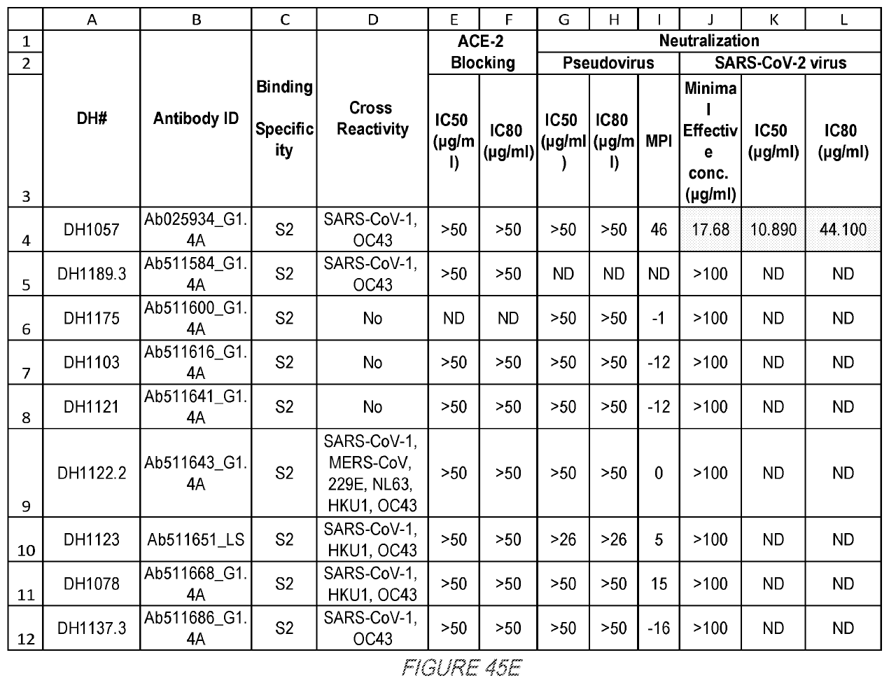

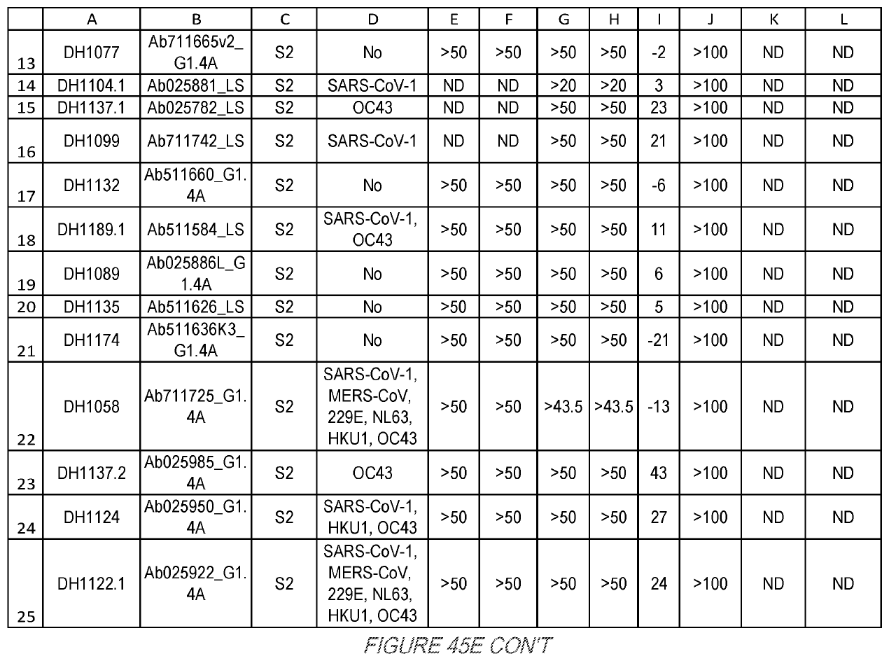

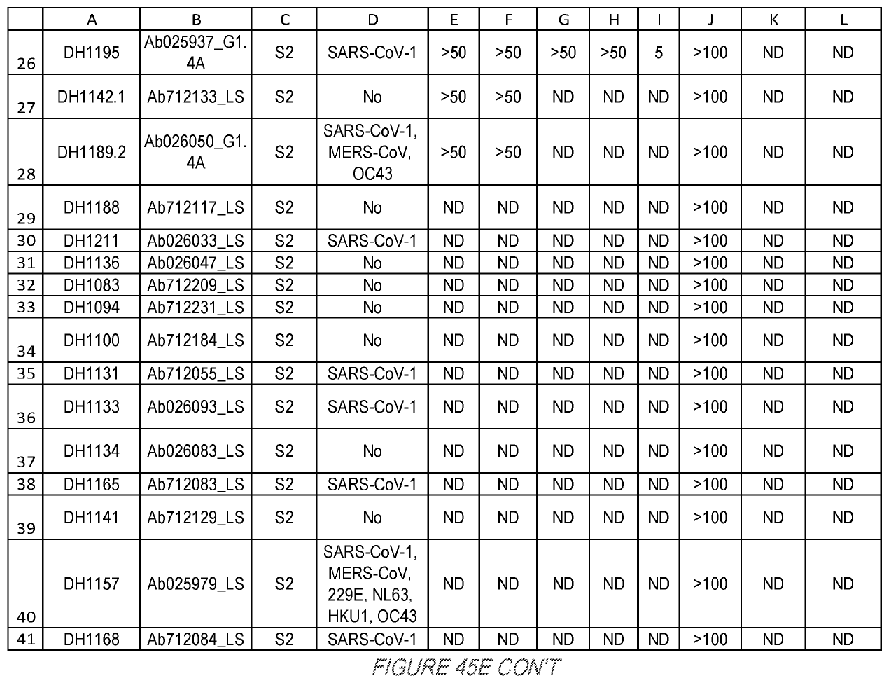

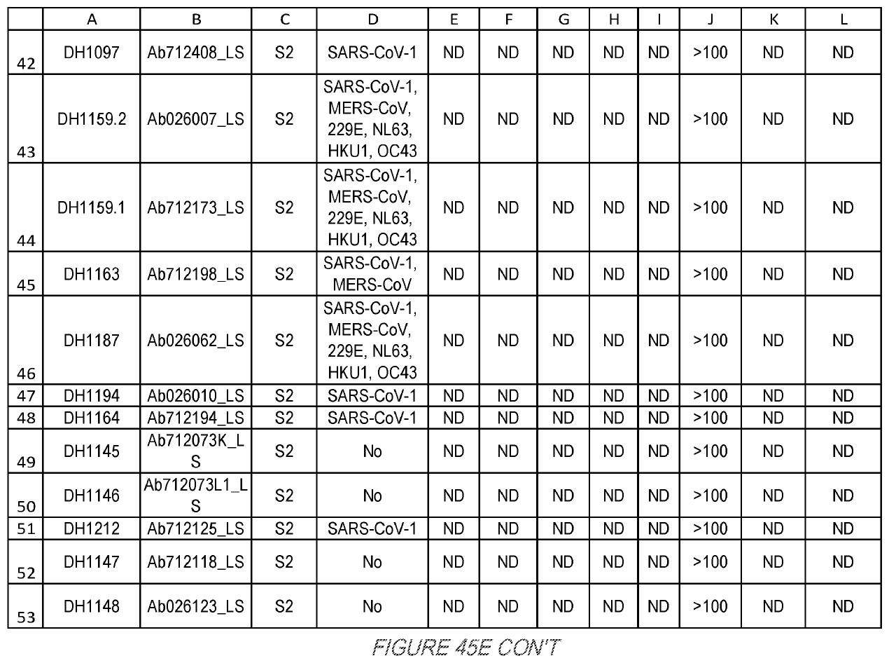

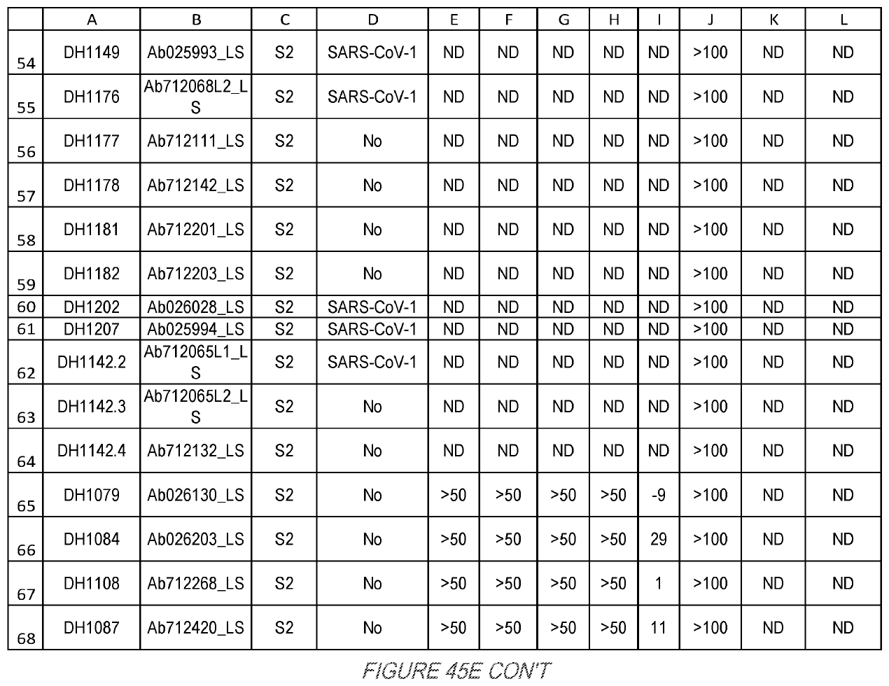

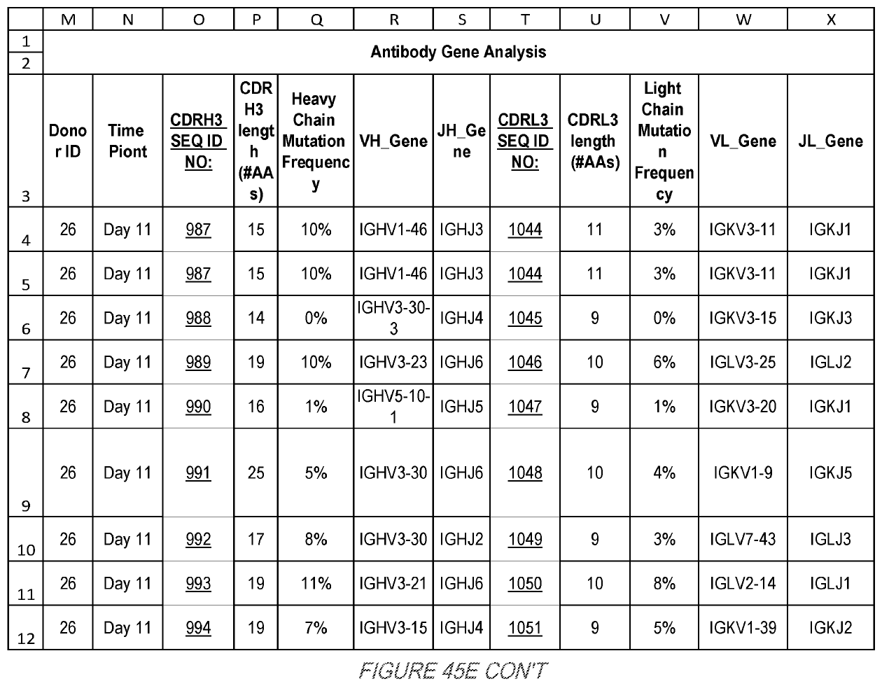

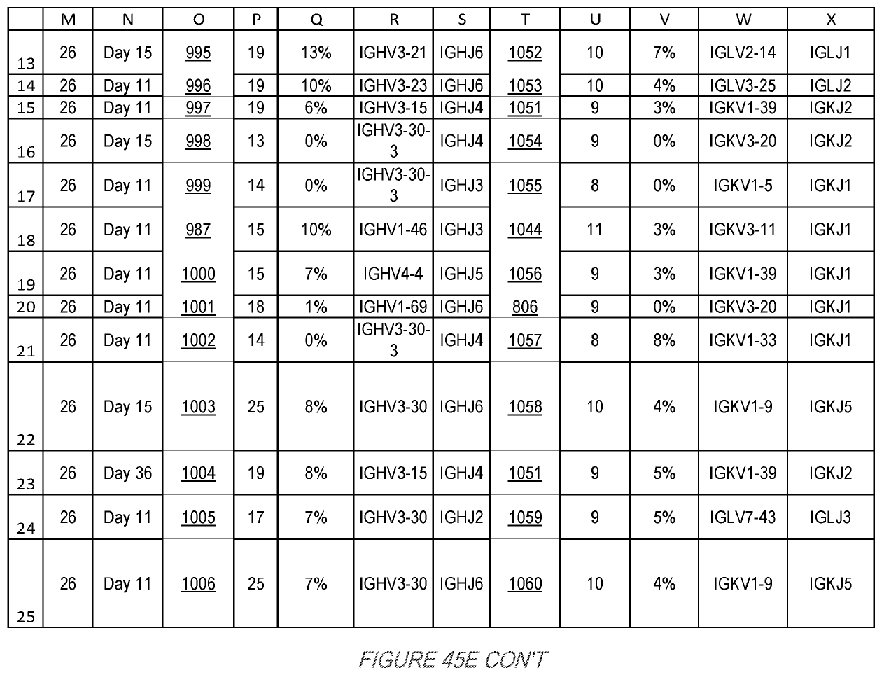

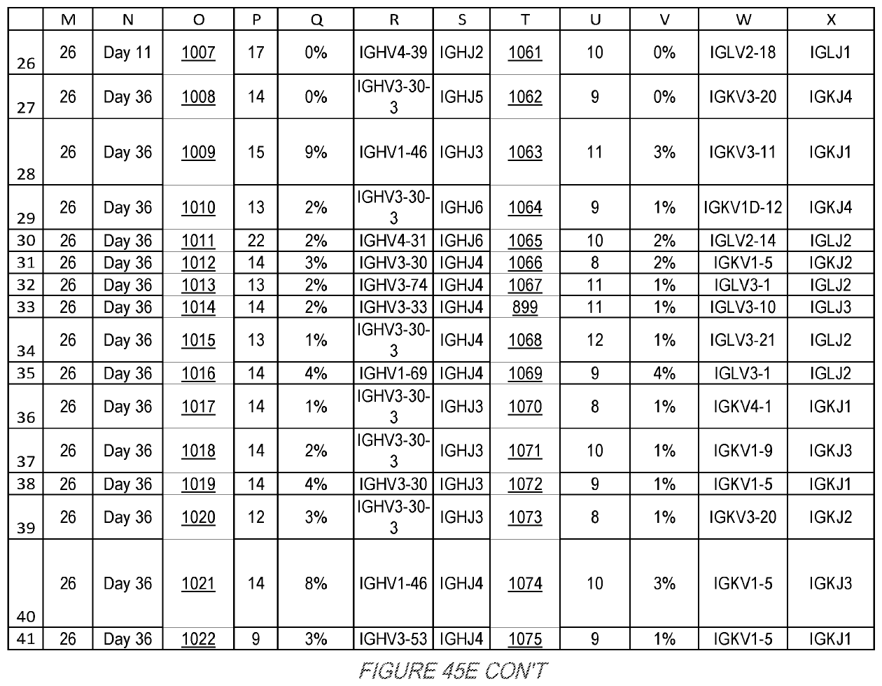

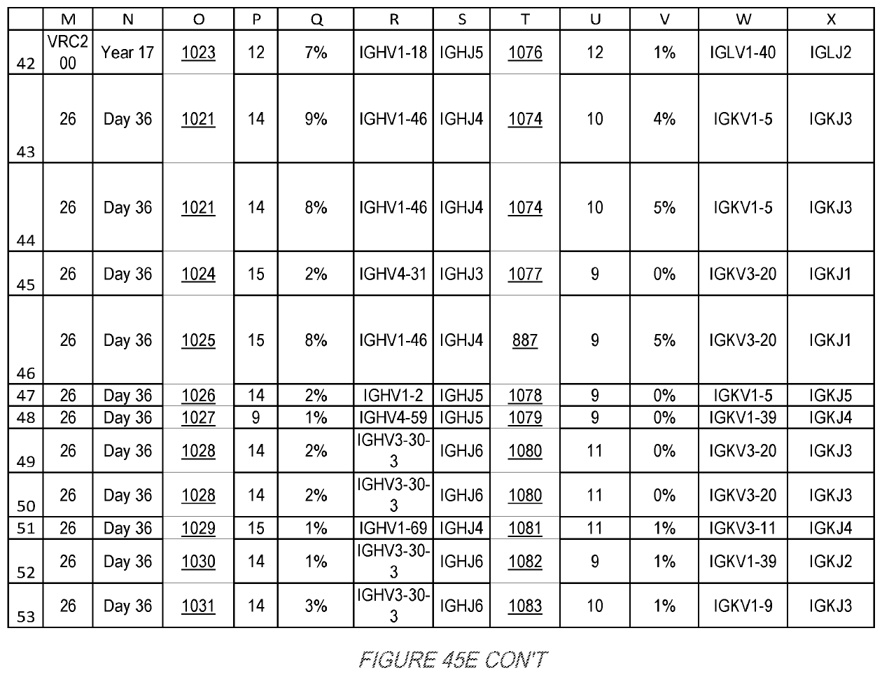

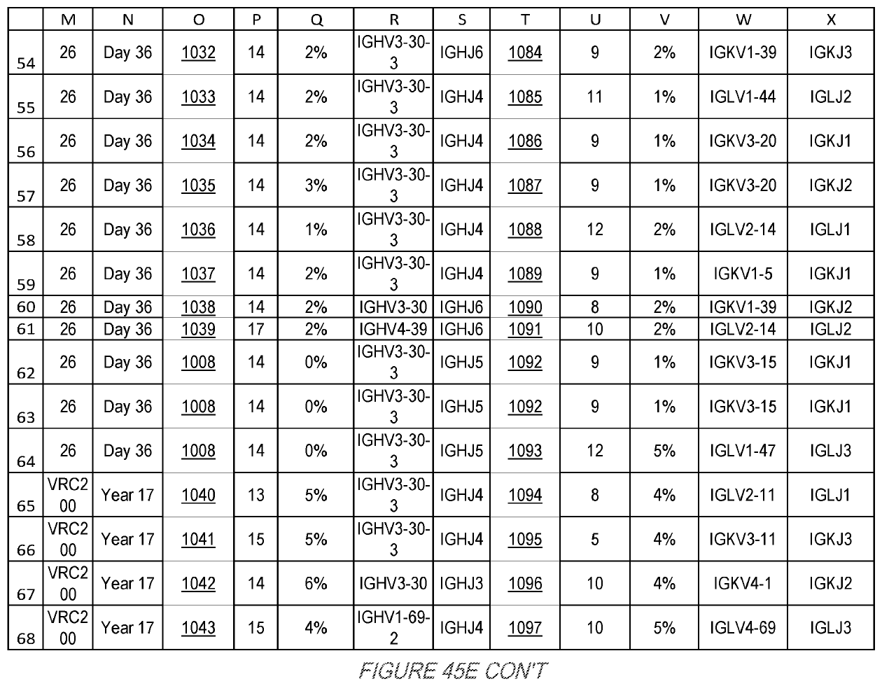

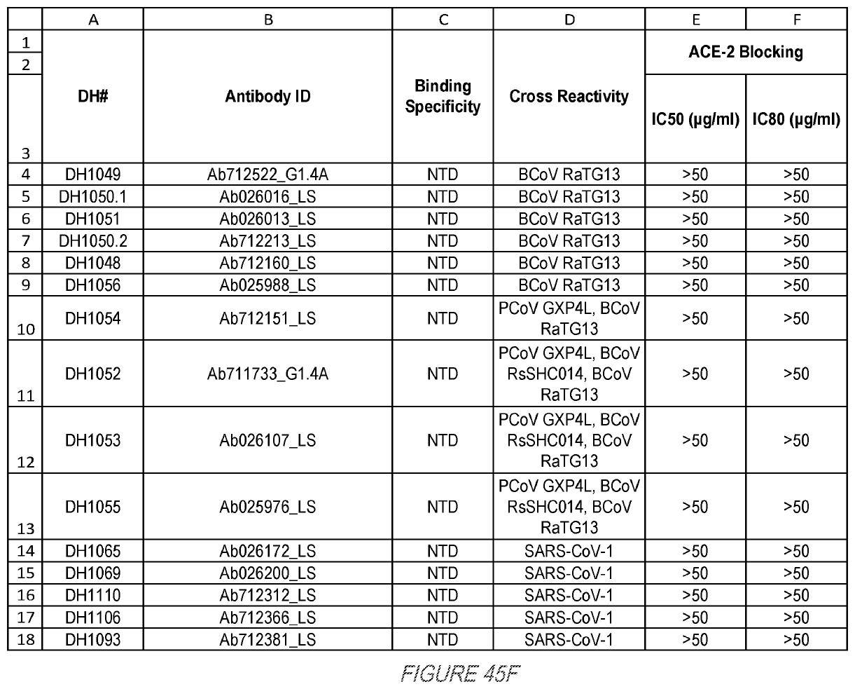

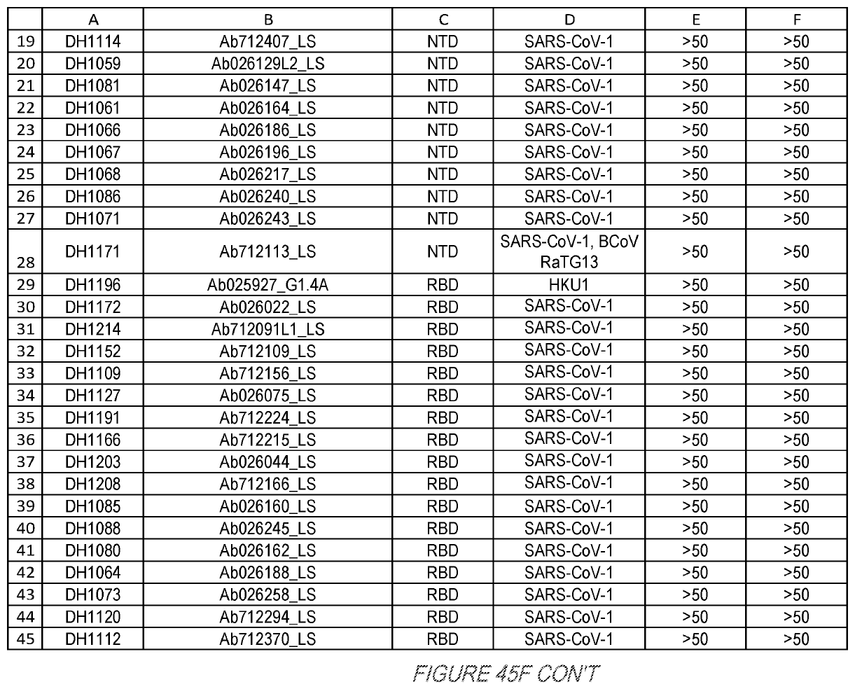

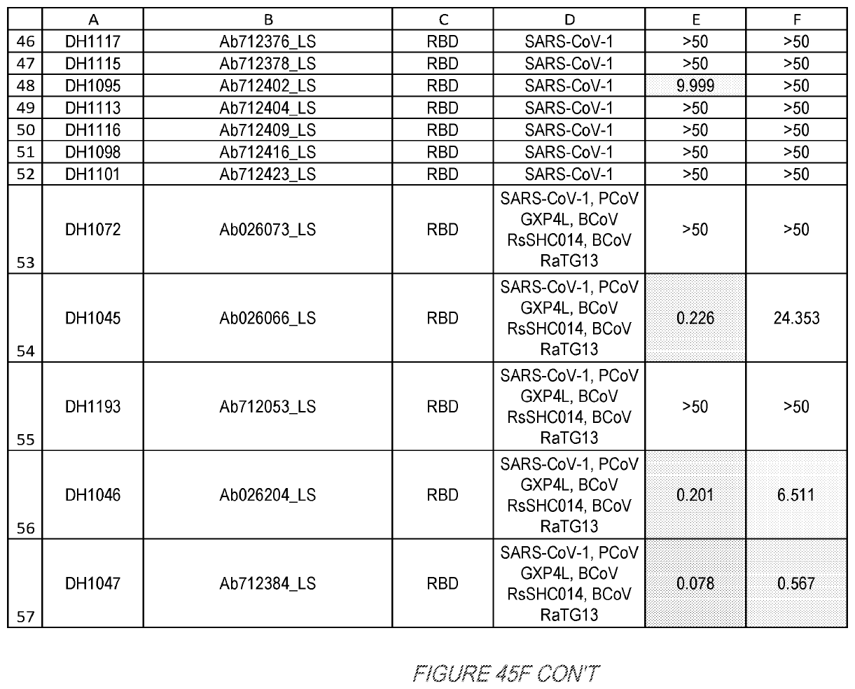

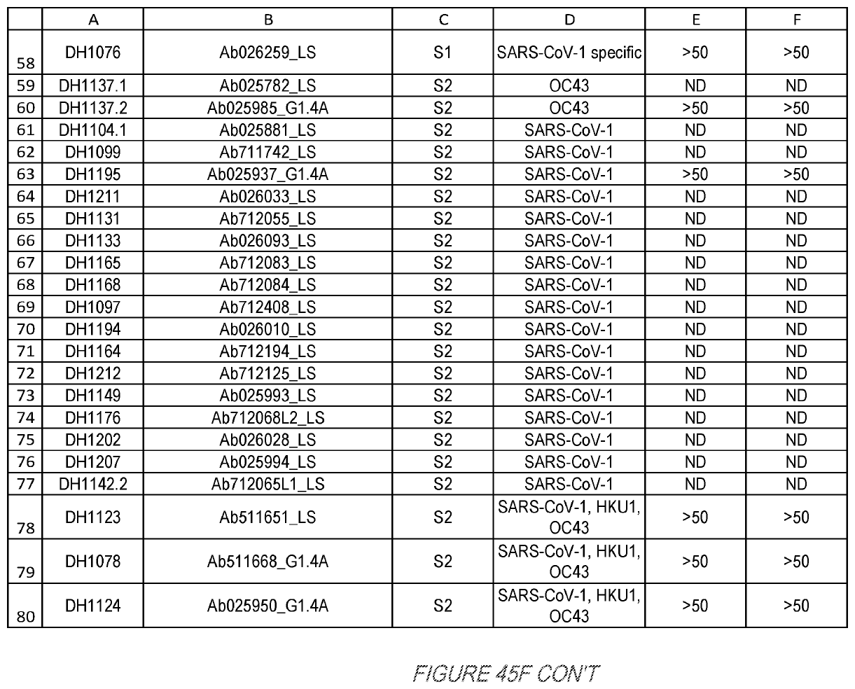

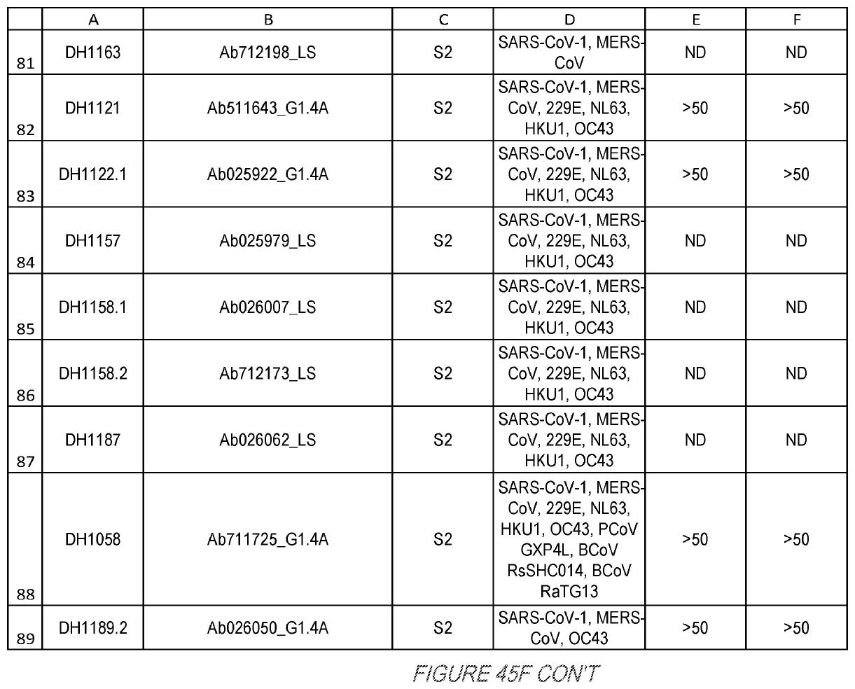

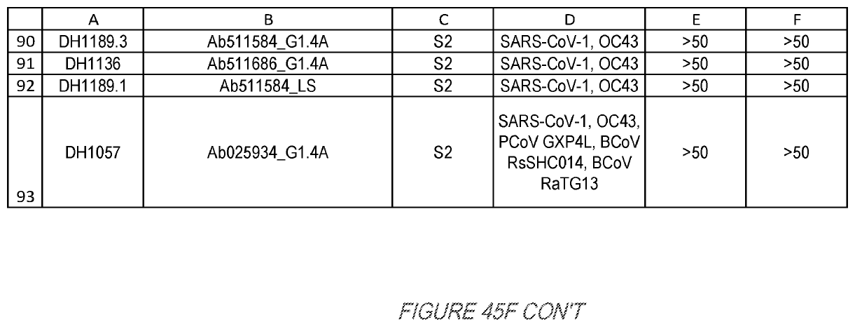

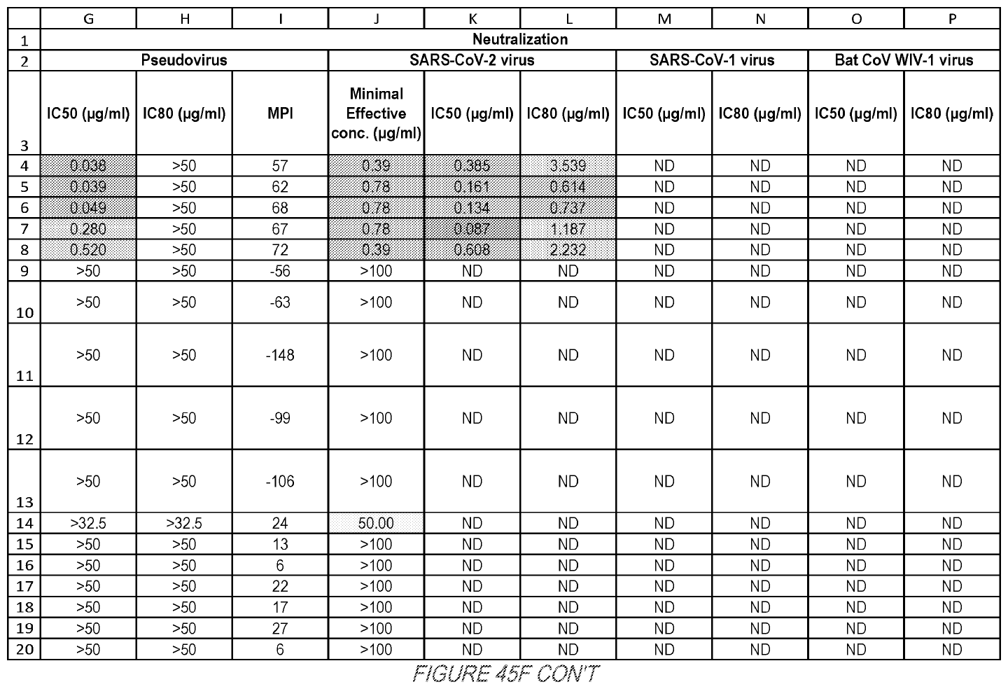

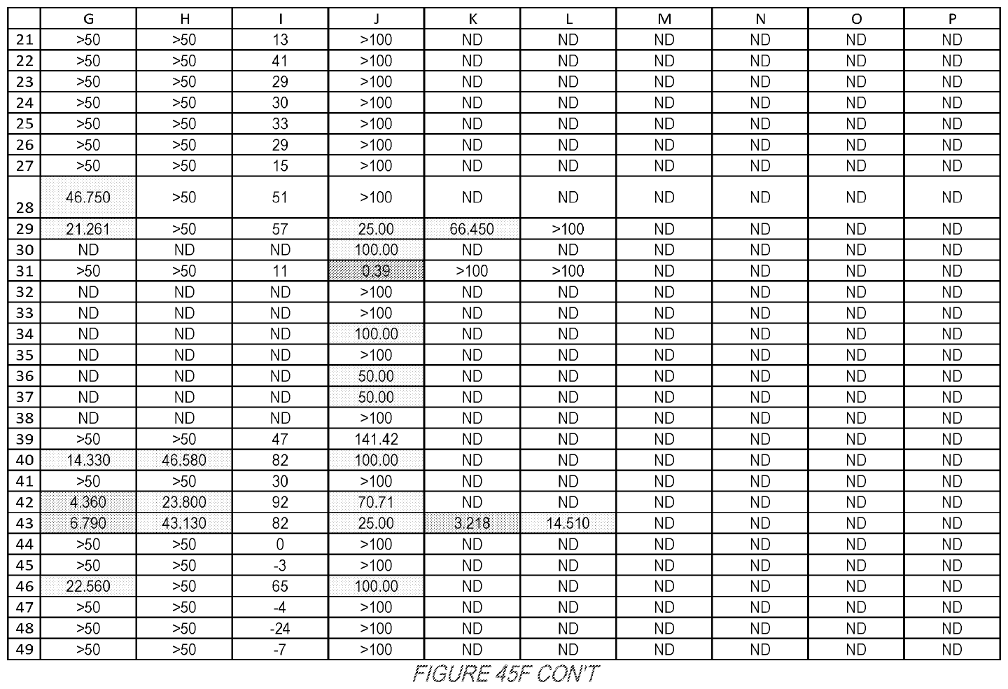

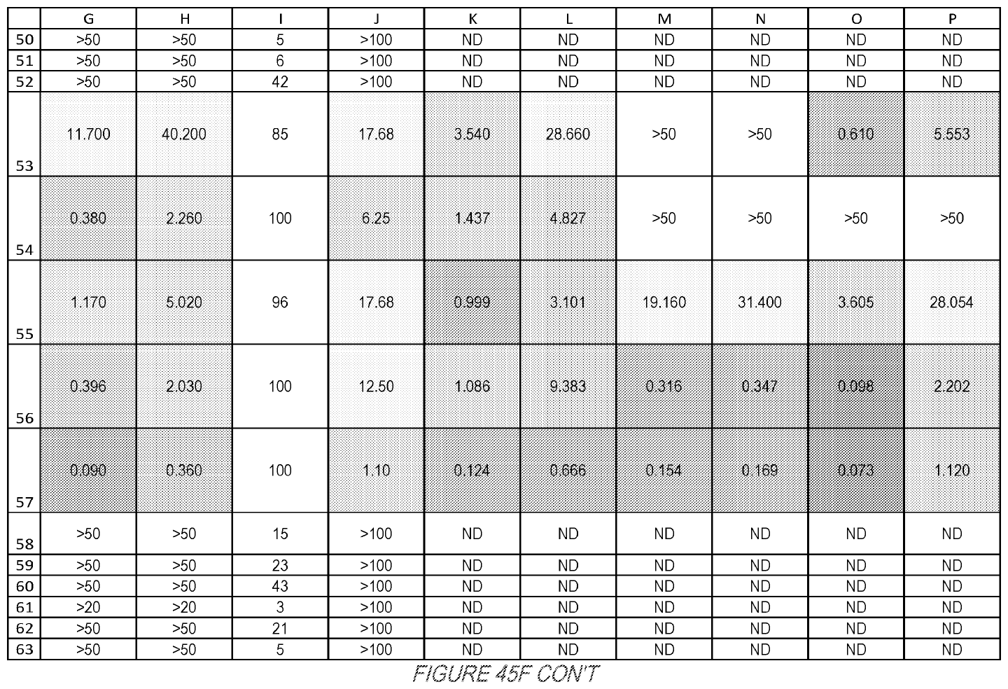

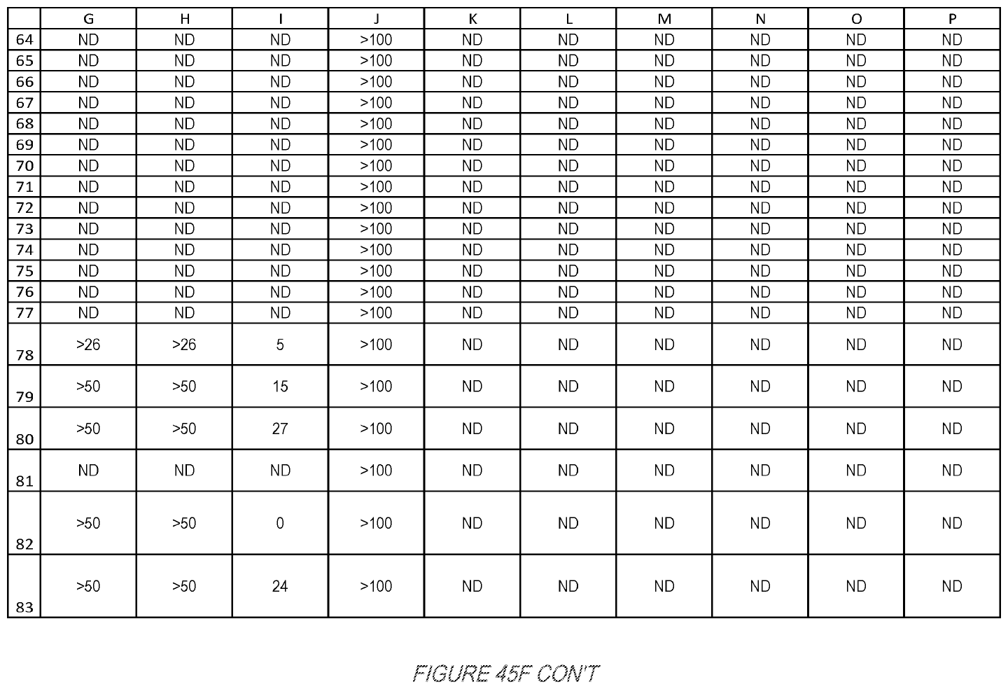

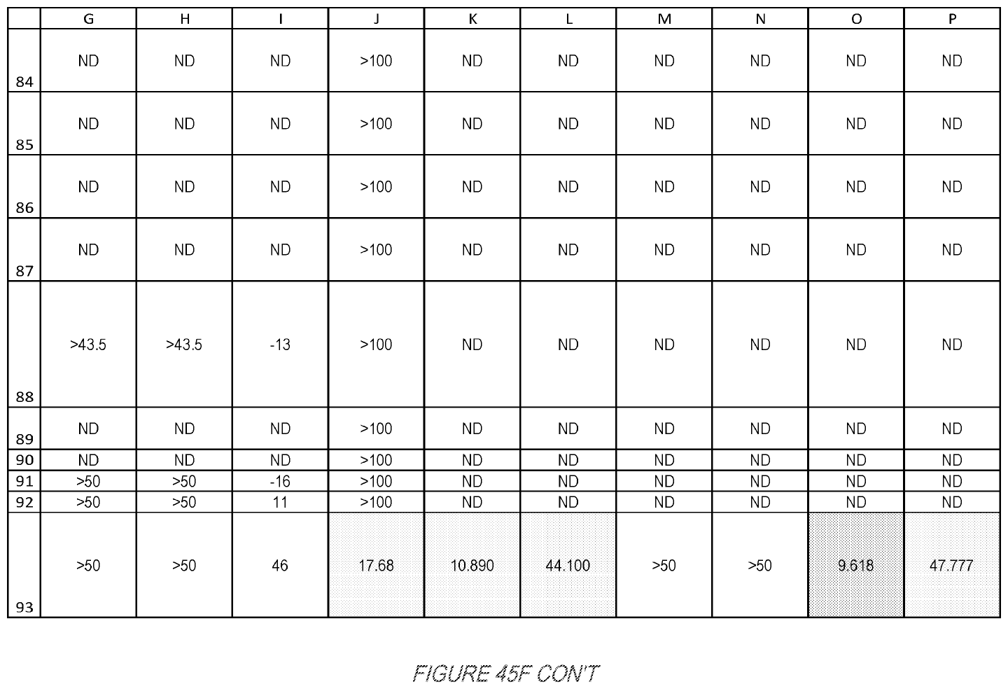

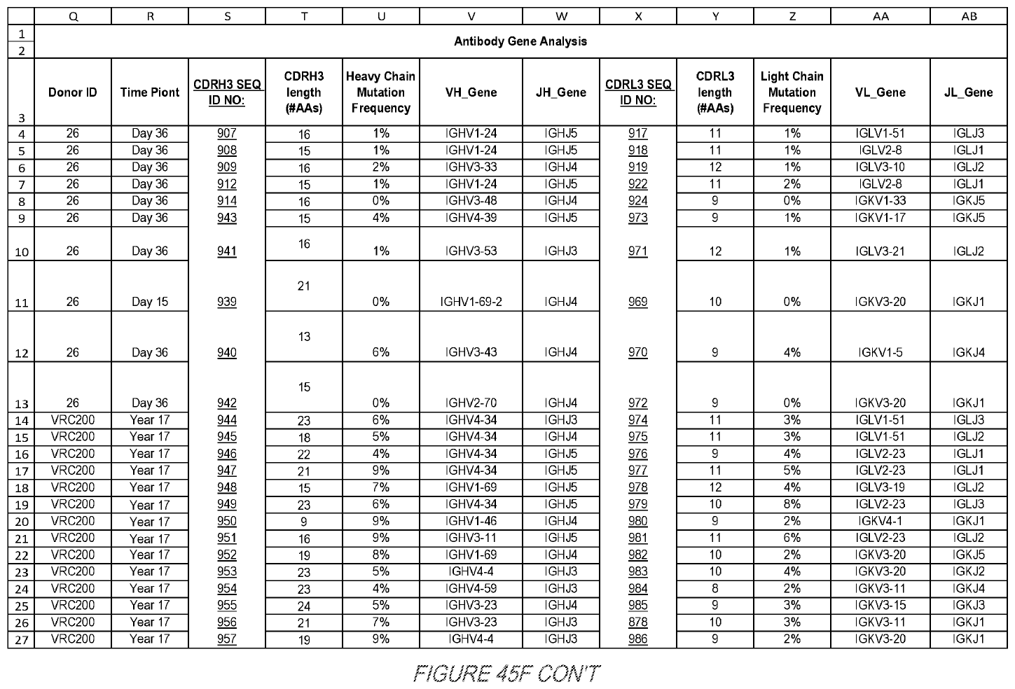

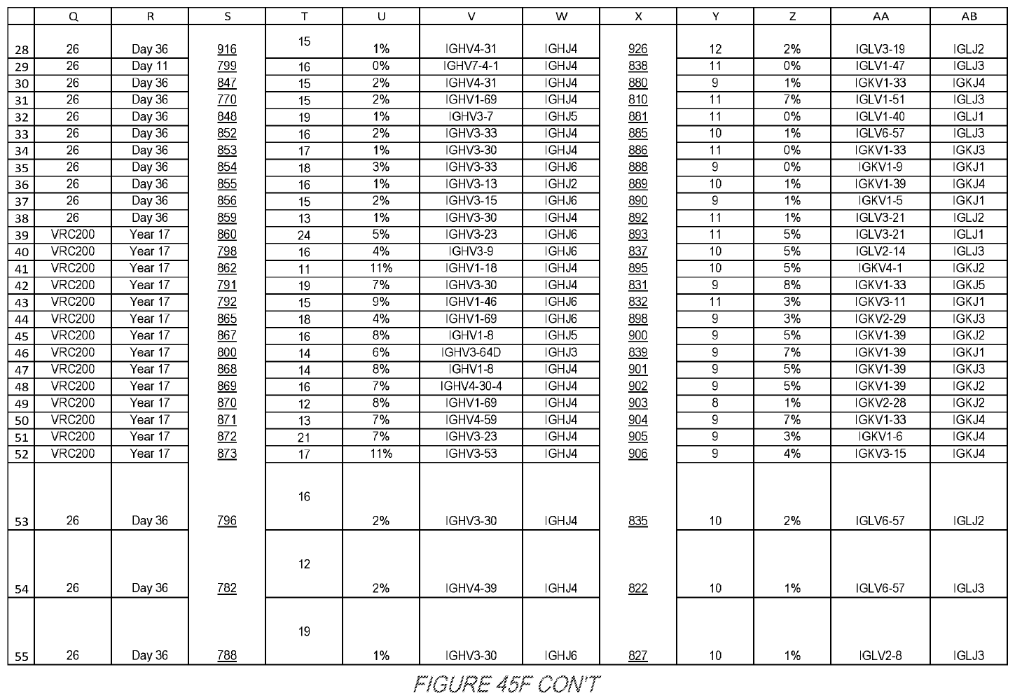

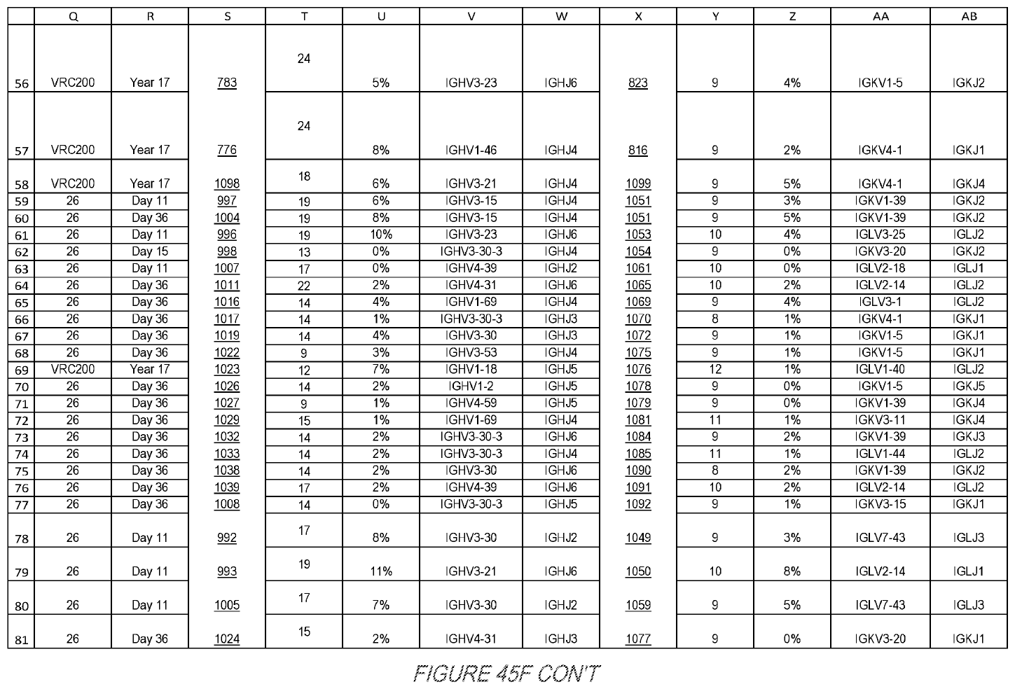

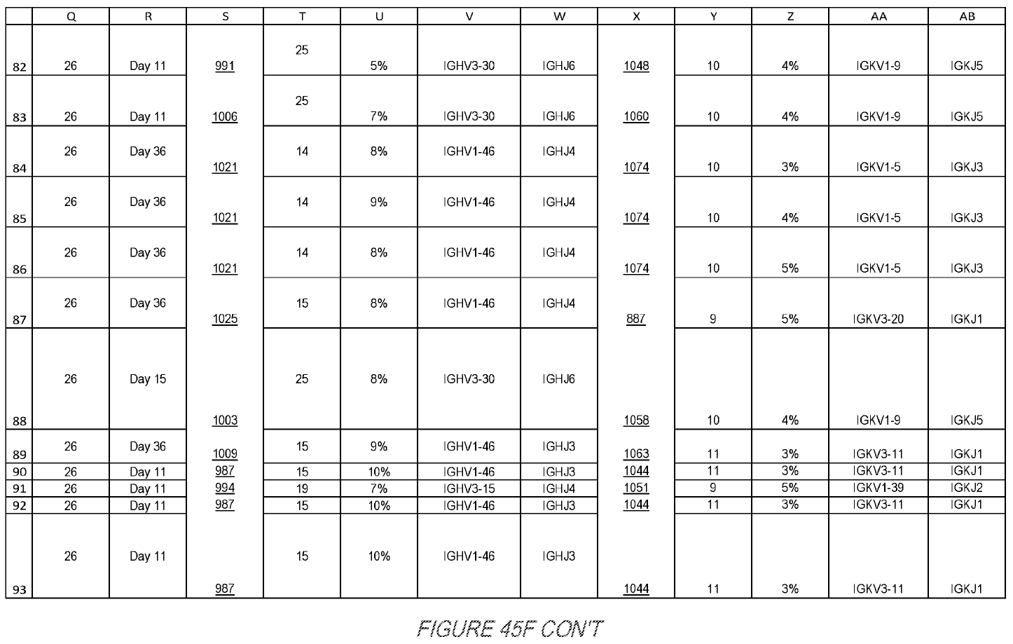

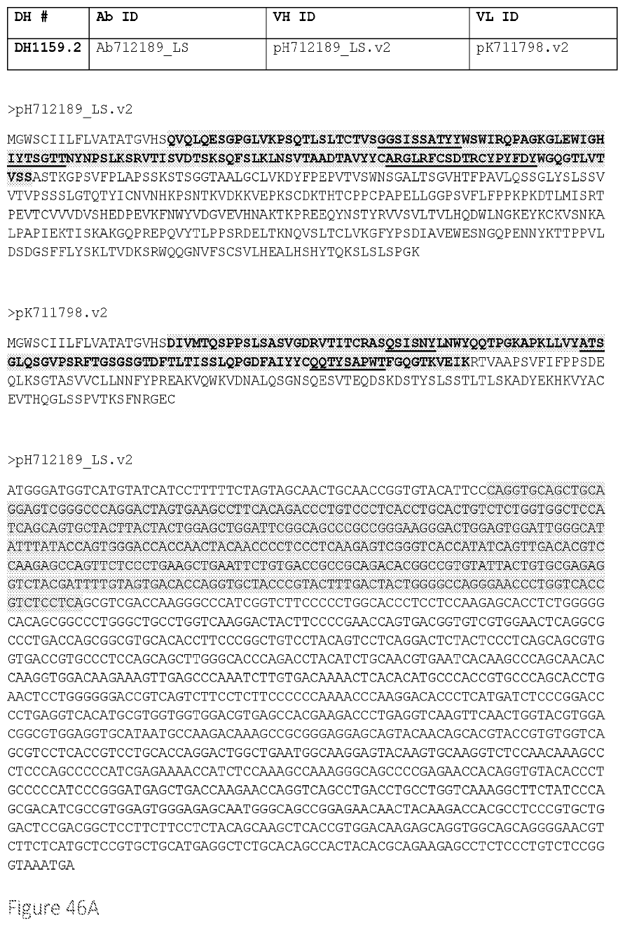

We performed similar comparative analyses of five FcγR-independent, infection-enhancing NTD antibodies, and five non-infection-enhancing NTD antibodies (DH1048. DH1049. DH1051. DH1050.1 and DH1050.2) that neutralized SARS-COV-2 (pseudovirus IC50 titers 39-520 ng/ml: SARS-COV-2 plaque reduction IC50 titers 390-780 ng/mL) (FIG. 2A and FIGS. 11C-D). The Fabs of neutralizing antibodies DH1050.1 and DH1051 bound to stabilized S ectodomain with affinities of 16 and 19 nM respectively, whereas the infection-enhancing antibody DH1052 bound with 294 nM affinity (FIGS. 12-14). NSEM reconstructions obtained for nine of the ten NTD antibodies showed that the FcγR-independent, infection-enhancing NTD antibodies (DH1053-DH1056) bound to S with their Fab constant domains directed downward toward the virus membrane (FIG. 2D), whereas the five neutralizing NTD-directed Abs (DH1048-DH1051) bound to S with the constant domain of the Fab directed upward away from the virus membrane (FIG. 2E). Thus. S protein antibody epitopes and binding modes were associated with FcγR-independent, infection-enhancing activity of NTD antibodies. The five neutralizing antibodies bound the same epitope as antibody 4A8 (17), with three of the five having the same angle of approach as 4A8 (FIG. 17). Interestingly, the NTD antibodies with the same angle of approach as 4A8, were also genetically similar to 4A8, being derived from the same Vul-24 gene segment (FIG. 45), although their light chains were different (17). These antibodies can constitute a neutralizing antibody class that can be reproducibly elicited upon SARS-COV-2 infection.

### Competition Between Infection-Enhancing and Non-Infection Enhancing Antibodies

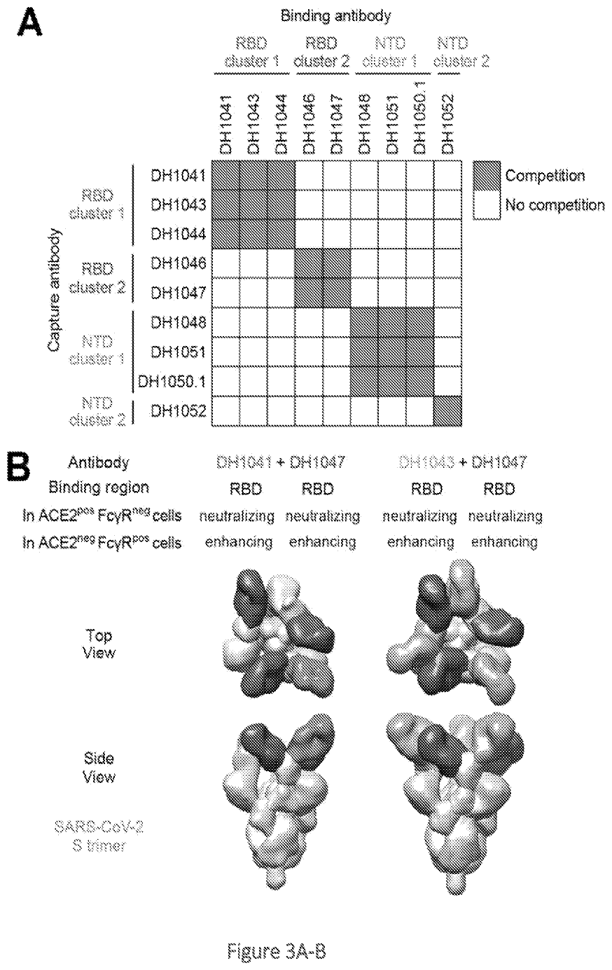

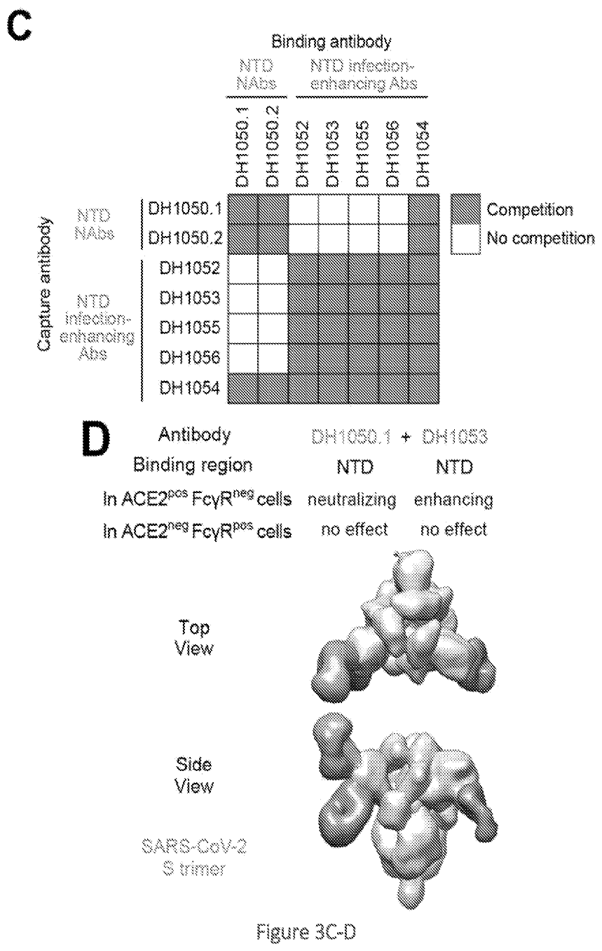

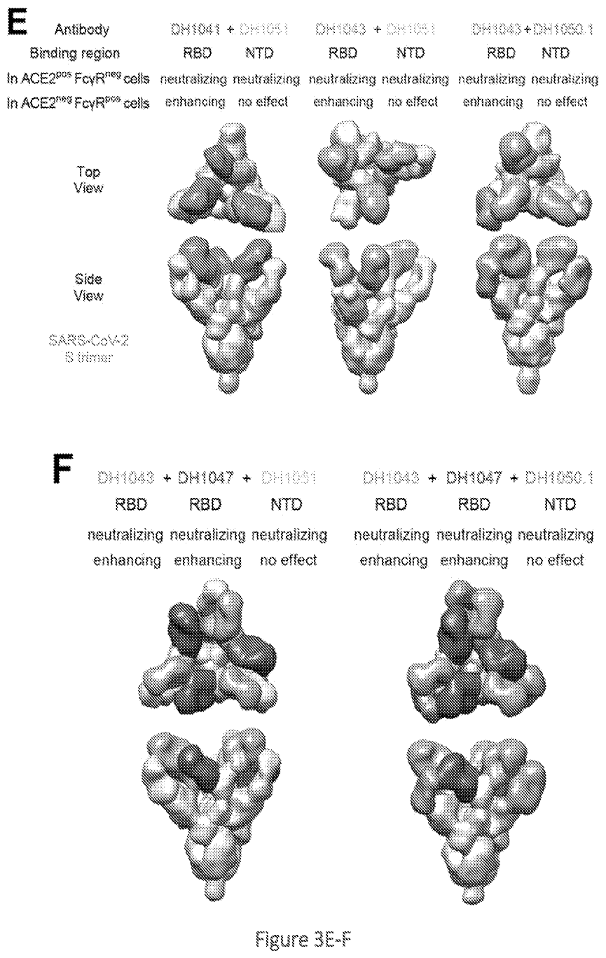

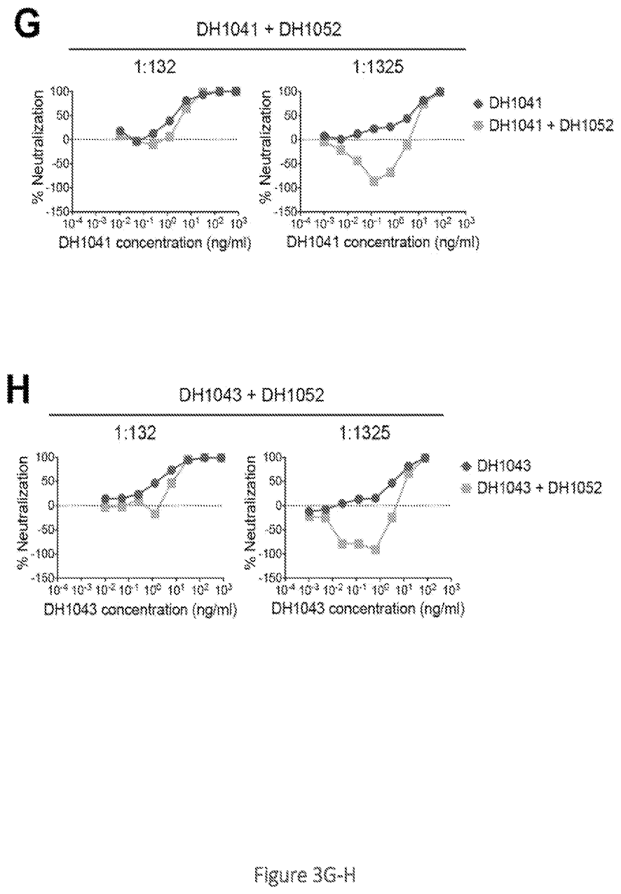

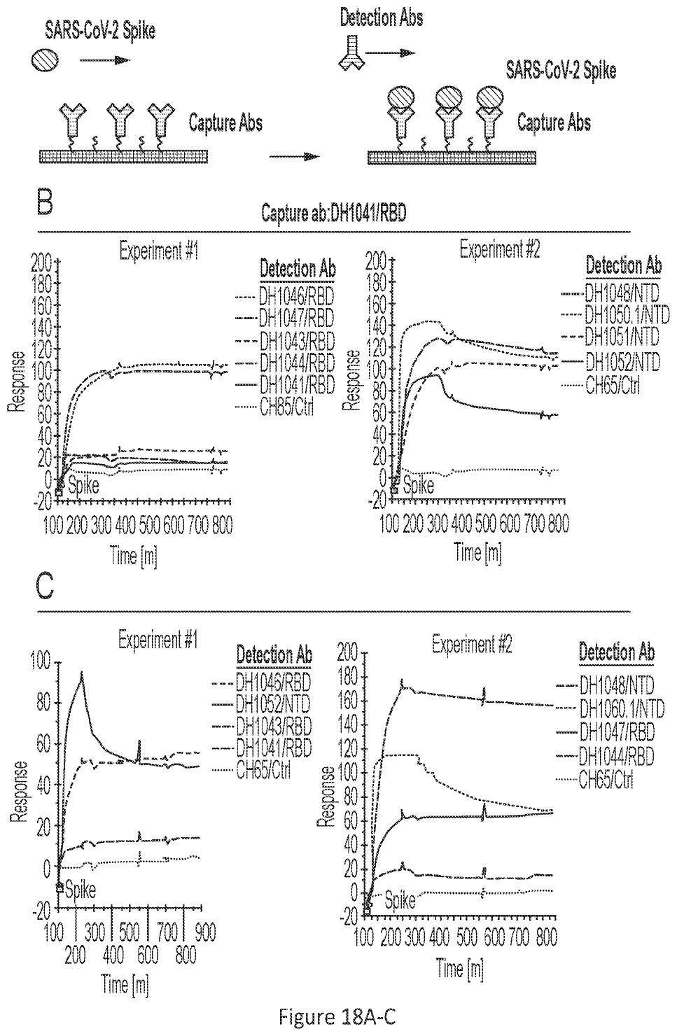

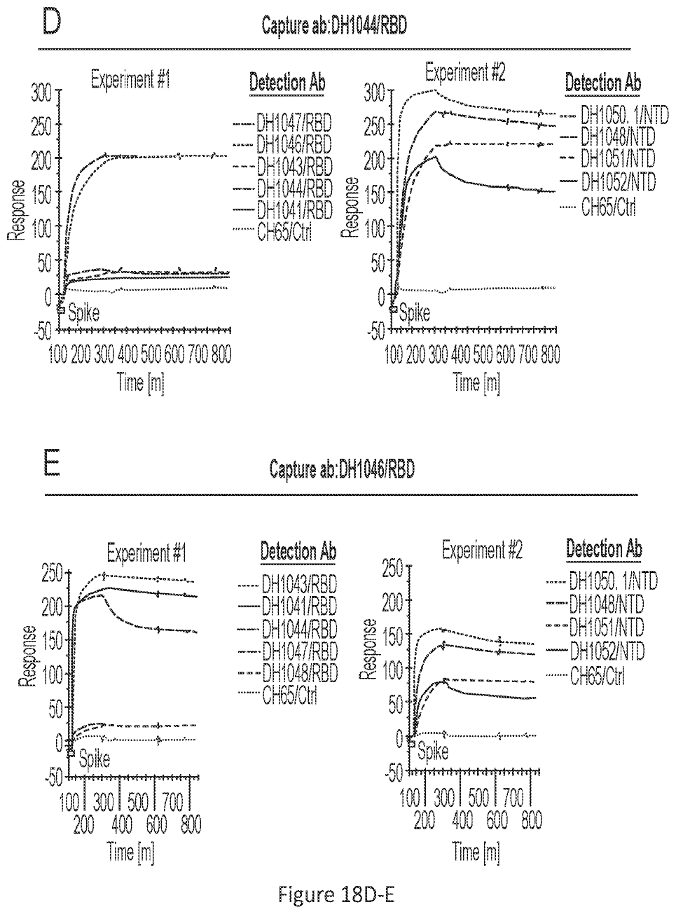

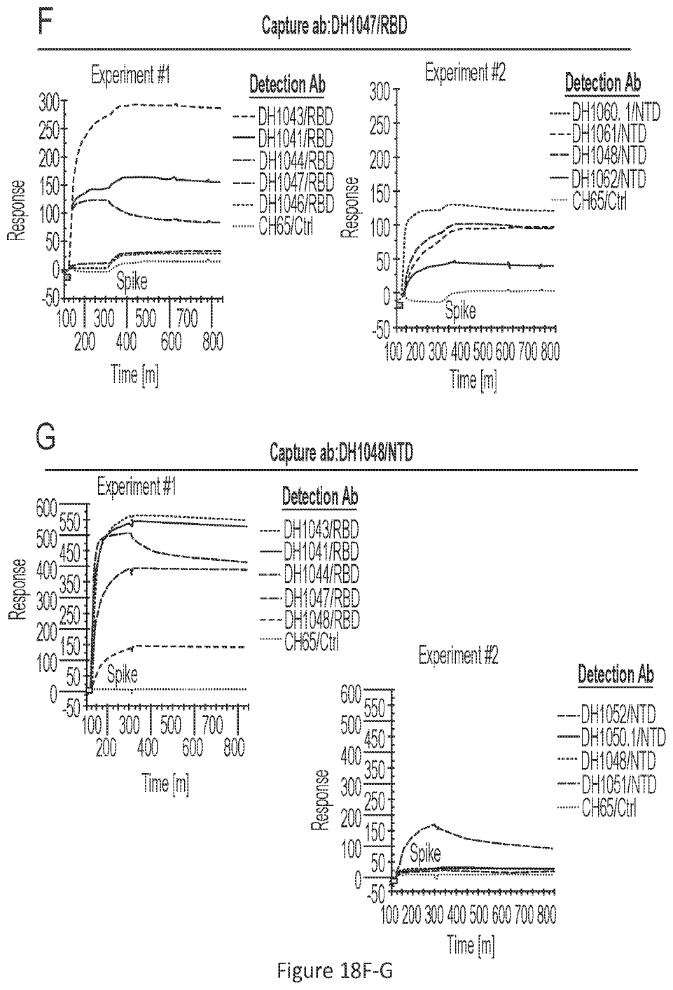

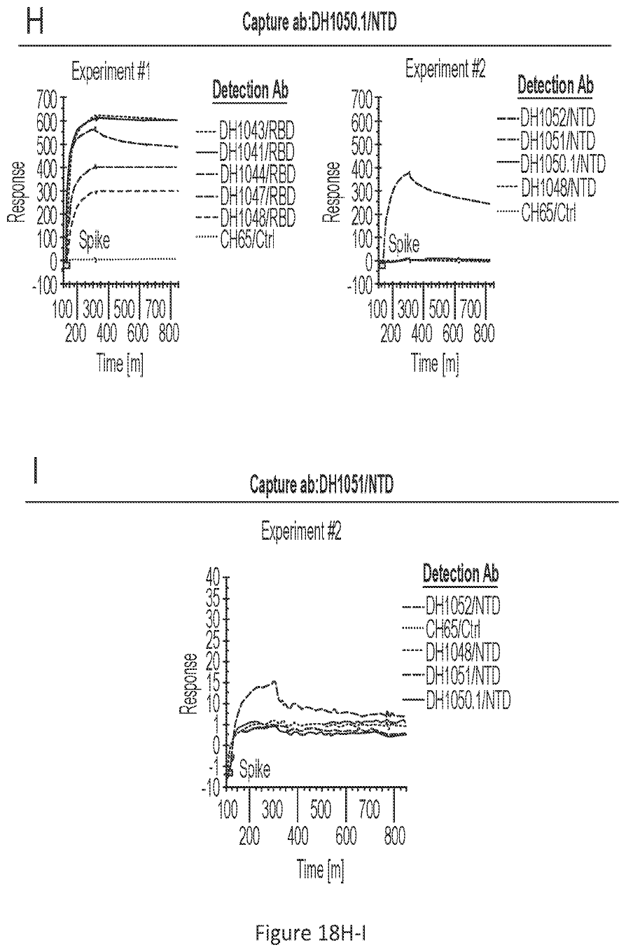

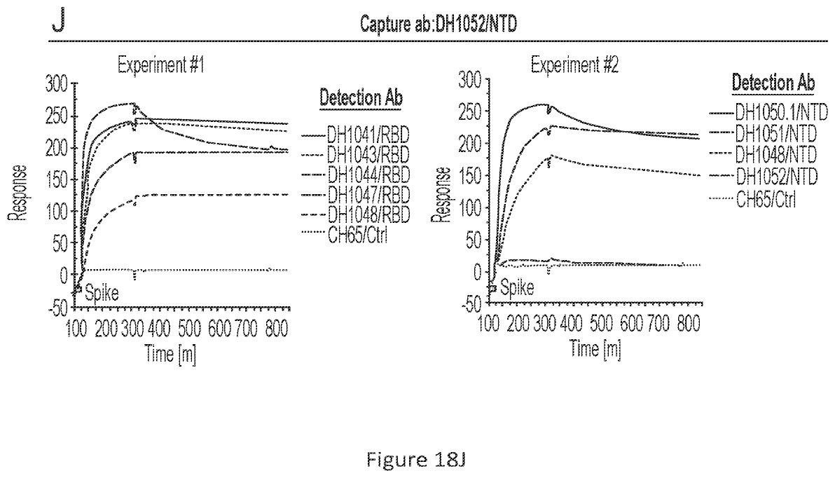

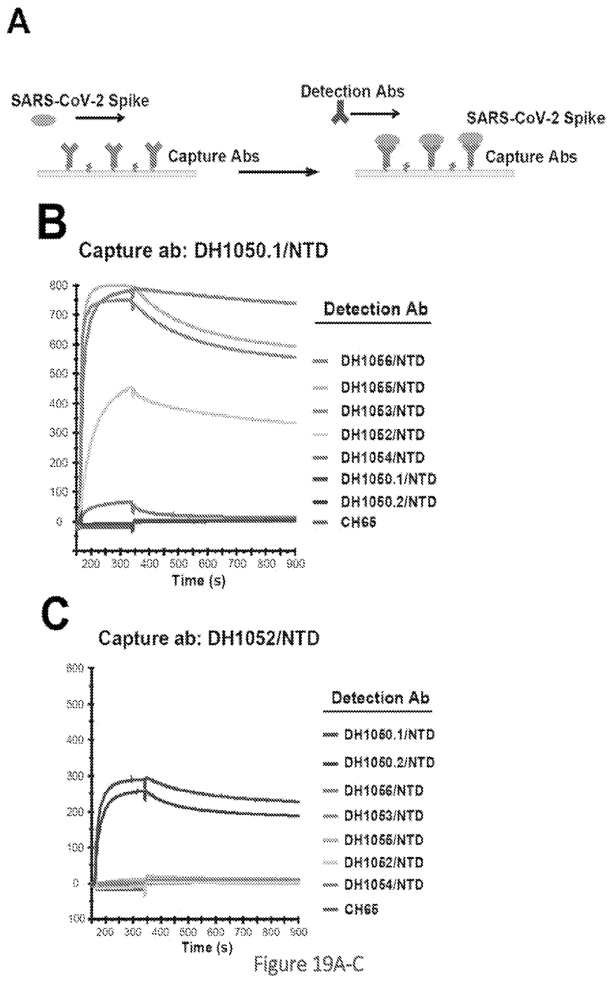

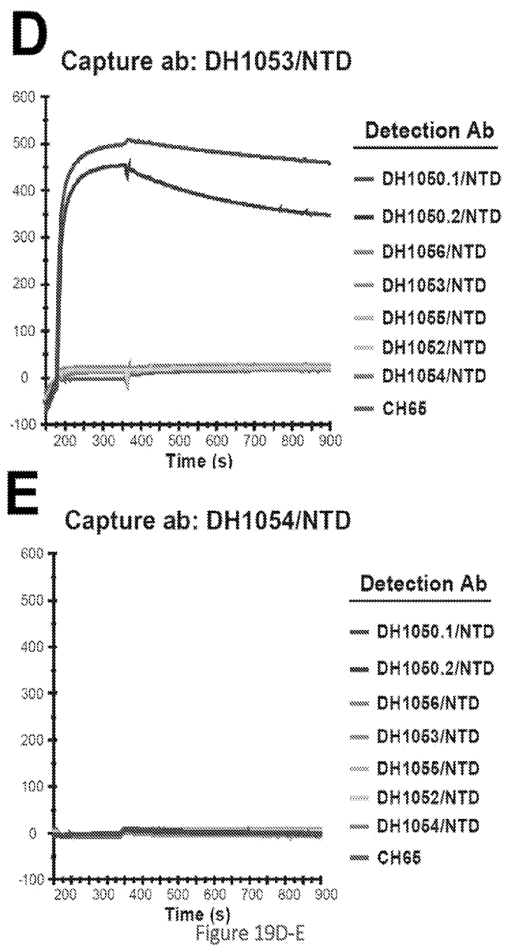

To determine whether infection-enhancing antibodies can compete with non-infection-enhancing antibodies for binding to S ectodomain, we performed surface plasmon resonance (SPR) competitive binding assays. RBD antibodies segregated into two clusters, where antibodies within a cluster blocked each other and antibodies in different clusters did not block each other (FIG. 3A. FIG. 18). One cluster included antibodies DH1041. DH1043 and DH1044. and the other cluster included antibodies DH1046 and DH1047. NSEM reconstructions showed DH1041 and DH1047 Fabs bound simultaneously to different epitopes of the stabilized S trimer (FIG. 3B). DH1043 and DH1047 Fabs also bound simultaneously to different epitopes on the stabilized S protein (FIG. 3B).

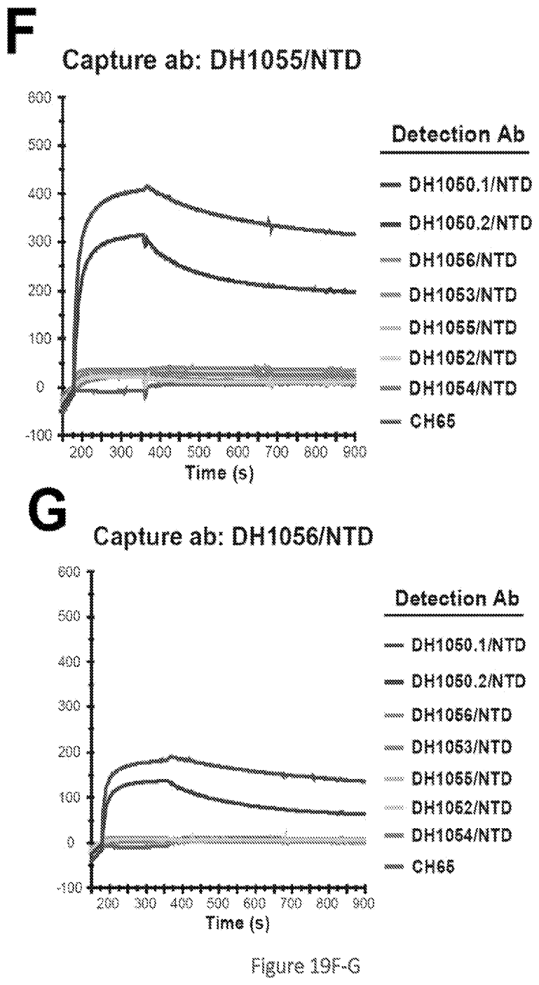

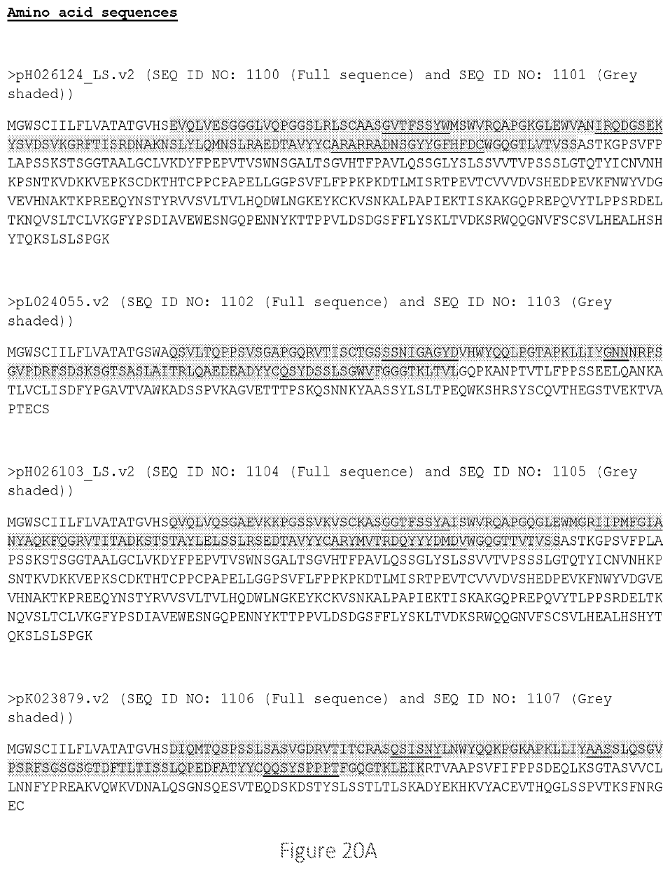

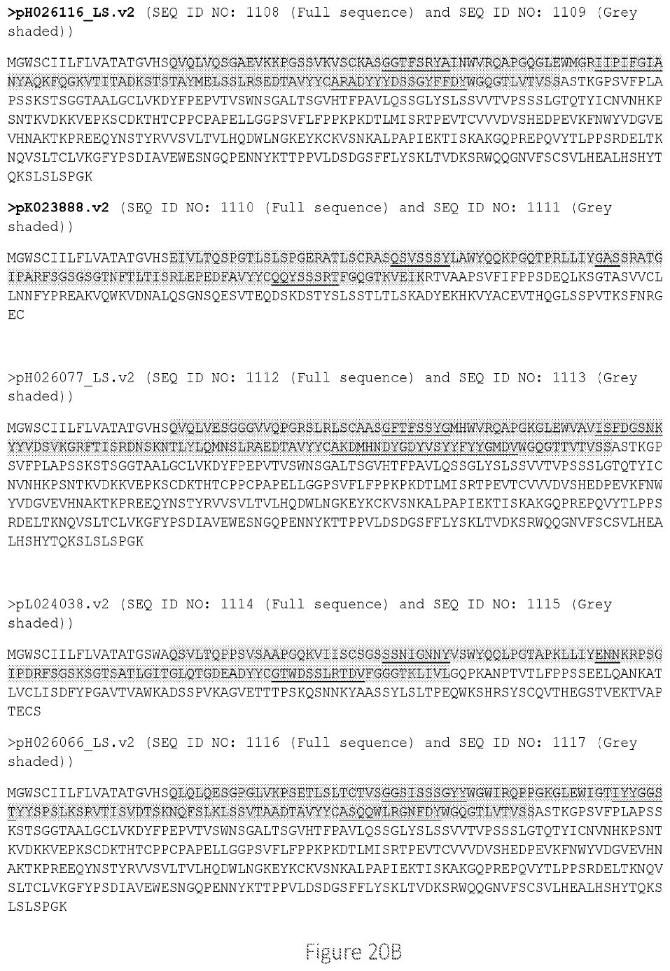

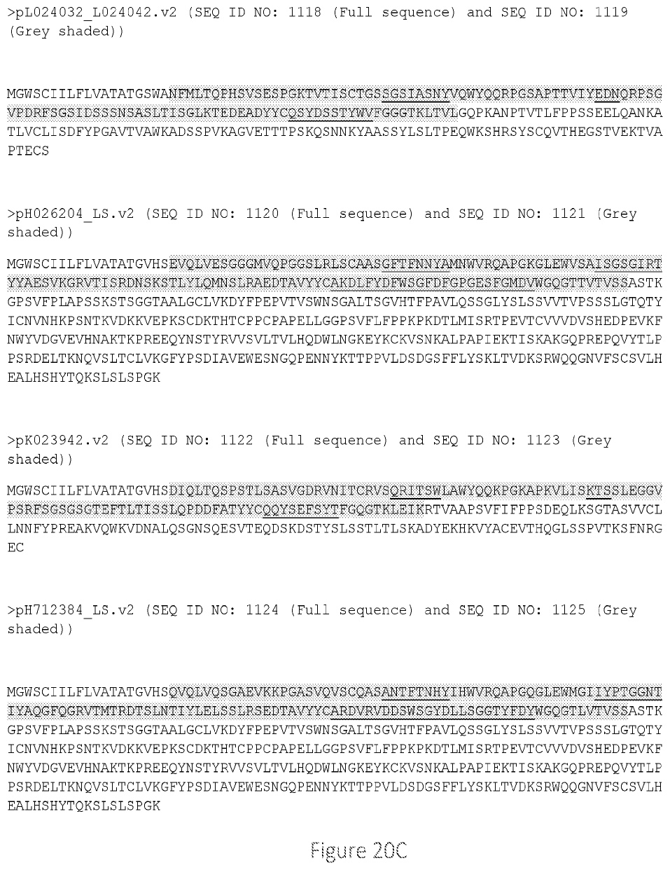

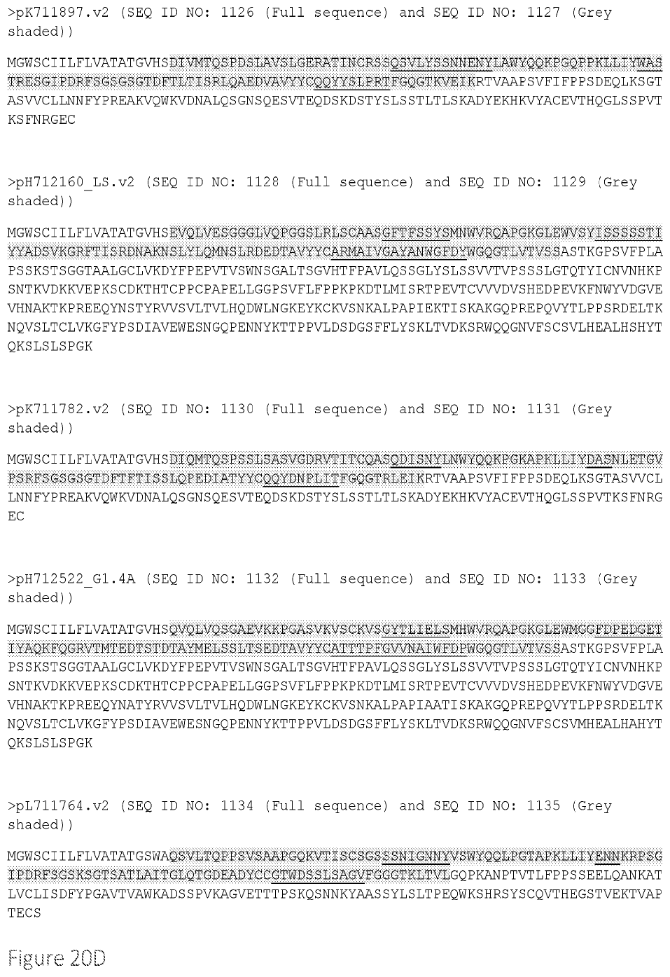

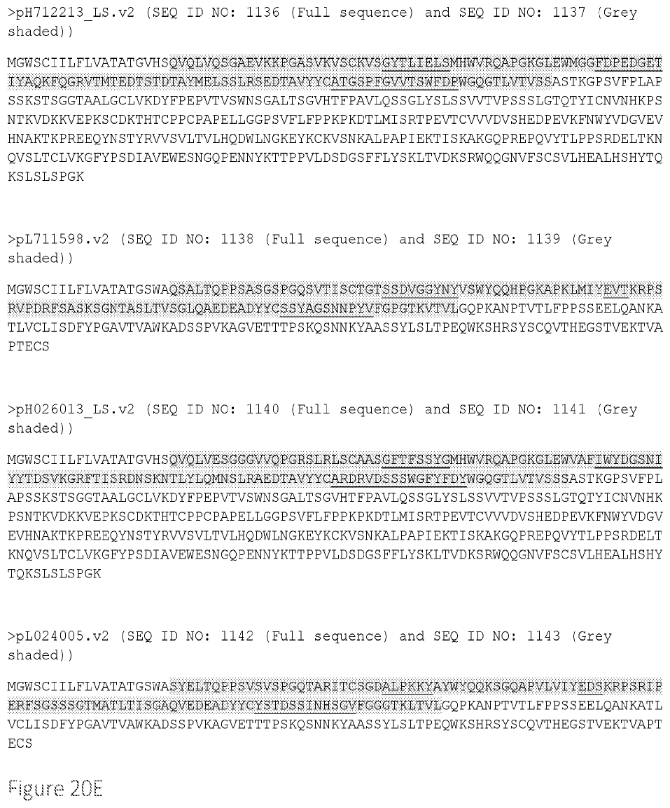

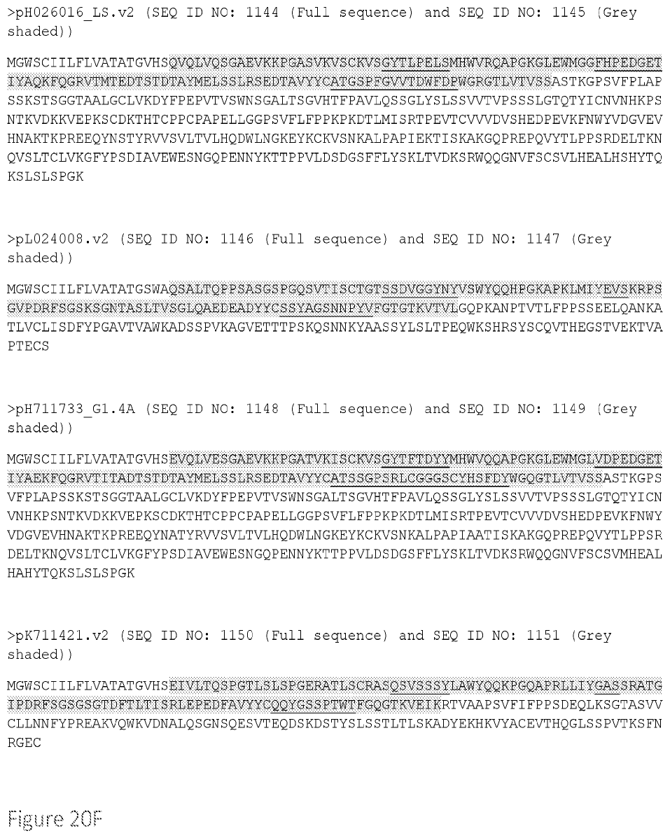

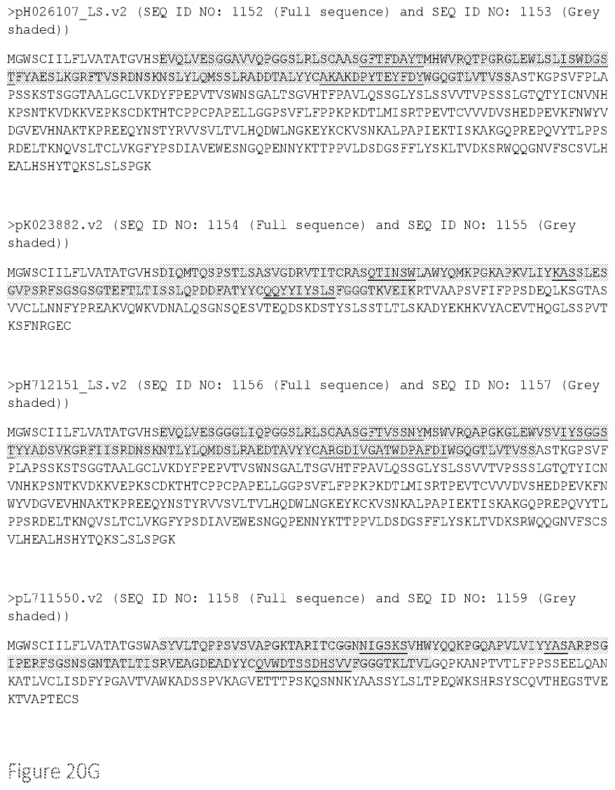

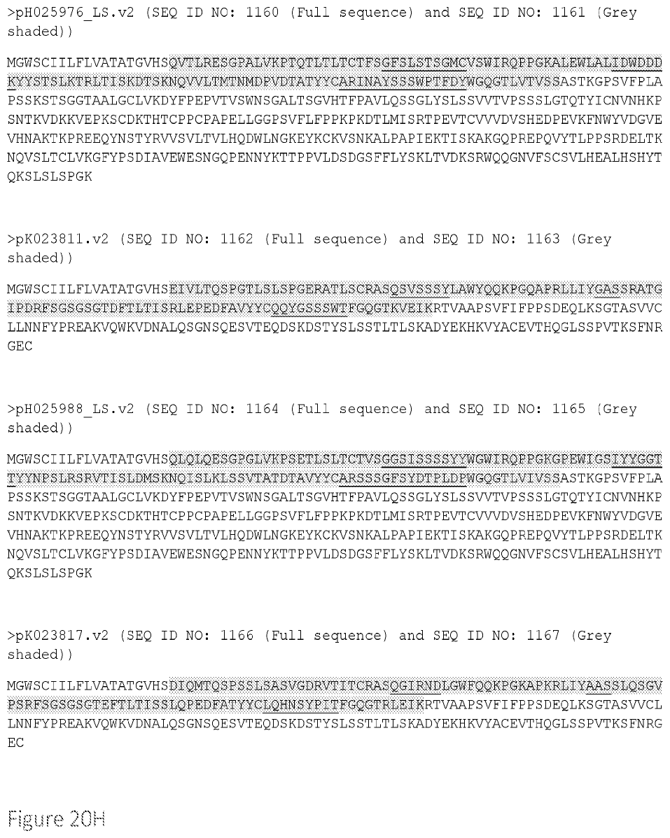

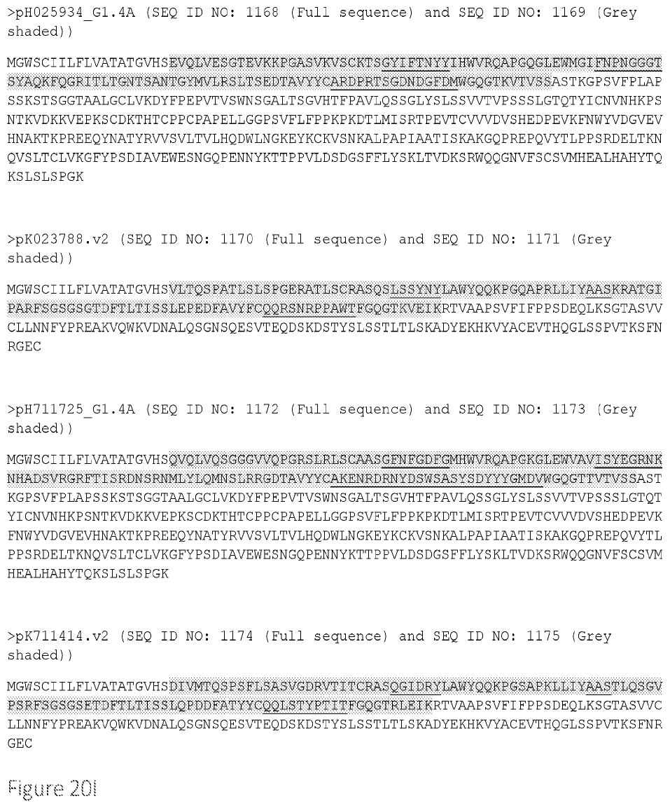

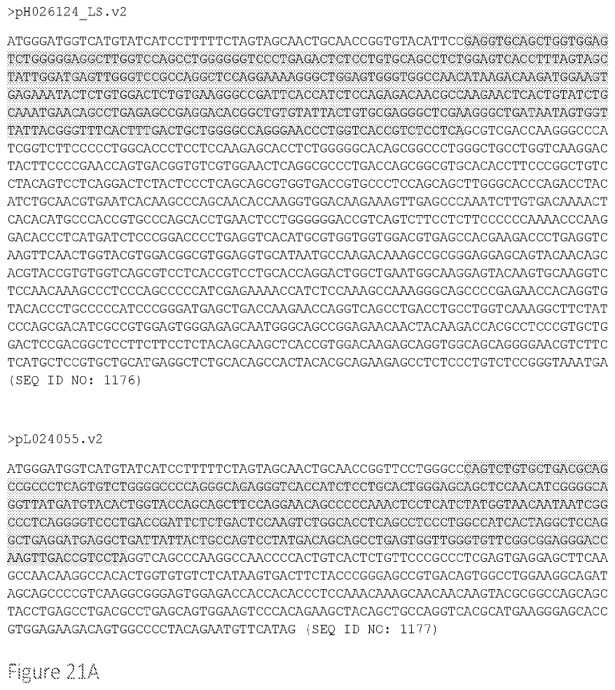

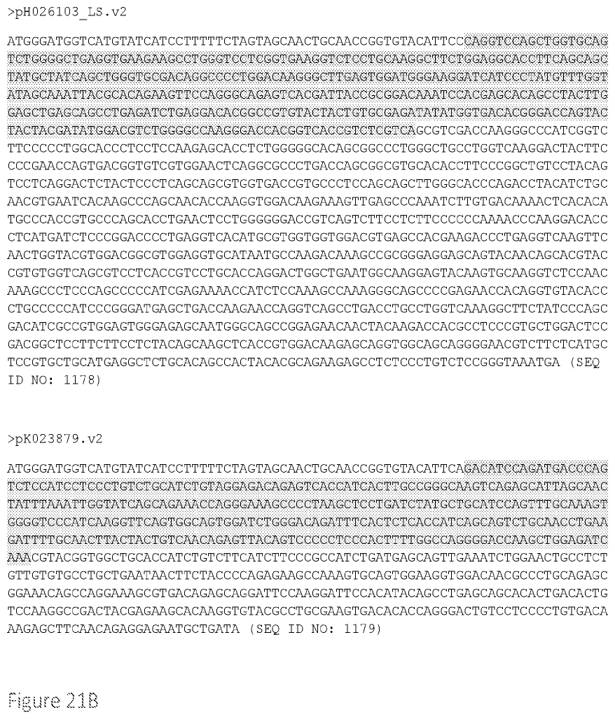

NTD antibodies also segregated into two clusters based on their ability to block each other (FIG. 3A). Neutralizing NTD antibodies blocked each other and formed one cluster, while infection-enhancing/non-neutralizing NTD antibodies blocked each other and formed a second cluster (FIGS. 3A and C, FIGS. 18 and 19). NSEM reconstruction of SARS-COV-2 S trimer bound with Fabs of neutralizing NTD antibody DH1050.1 and infection-enhancing NTD antibody DH1052 confirmed that the two antibodies can simultaneously bind to distinct epitopes on a single SARS-COV-2 S trimer (FIG. 3D). DH1054 was unique as it was able to block both infection-enhancing and neutralizing NTD antibodies (FIG. 3C, FIG. 19).

NTD antibodies did not compete with RBD antibodies for binding to S trimer (FIG. 3A). This result gave rise to the notion that in a polyclonal mixture of antibodies, the SARS-CoV-2 S trimer can bind both RBD and NTD antibodies. To determine whether this complex can form, we liganded SARS-COV-2 S trimer with Fabs of each type of antibody and visualized the complex using NSEM. NSEM showed that neutralizing RBD antibodies can also bind to the same S protomer as neutralizing NTD antibodies DH1050.1 or DH1051 (FIG. 3E). Moreover, we found that a single S protomer can be occupied by two RBD antibodies (DH1043 and DH1047) and an NTD antibody (DH1050.1) (FIG. 3F). Thus, the S trimer can simultaneously bind to multiple RBD and NTD neutralizing antibody Fabs.

### FcγR-Independent Infection-Enhancement in the Presence of Neutralizing Antibodies

The NSEM determination of antibody binding modes demonstrated that certain infection-enhancing antibodies and non-infection enhancing antibodies bound to distinct epitopes on the same S protomer (FIG. 3A-F). Thus, we determined the functional outcome of infection-enhancing antibodies binding to S in the presence of neutralizing antibodies. We examined whether a FcγR-independent, infection-enhancing NTD antibody can inhibit RBD antibody neutralization of ACE-2-mediated SARS-COV-2 pseudovirus infection of 293T/ACE2 cells in vitro. Without wishing to be bound by theory, the outcome will be dependent on which antibody was present at the highest concentration. We examined RBD antibody DH1041 neutralization in the presence of 132 and 1.325-fold excess of infection-enhancing NTD antibody DH1052. DH1041 neutralization activity was minimally decreased in the presence of 132-fold excess of DH1052. When DH1041 neutralization was assessed in the presence of 1.325-fold excess of antibody DH1052, infection enhancement was observed when DH1041 concentration was below 10 ng/ml (FIG. 3G, FIG. 26). A nearly identical result was obtained when we examined neutralization by DH1043 (FIG. 3H, FIG. 26). Thus, a ˜1000-fold excess of infection-enhancing NTD antibody was required to out-compete a potent RBD neutralizing antibody in vitro.

### Cryo-EM Structural Determination of RBD and NTD-Directed Antibody Epitopes

To visualize atomic level details of their interactions with the S protein, we selected representatives from the panels of RBD and NTD-directed antibodies for structural determination by cryo-EM. Of the RBD-directed antibodies, we selected two (DH1041 and DH1043) that most potently neutralized SARS-COV-2 virus in the 293T/ACE2 pseudovirus assay and also enhanced infection in TZM-bl-FcγRI or—FcγRIIb cells. We also selected the RBD-directed antibody DH1047, which shared the infection enhancing and ACE-2 blocking properties of DH1041 and DH1043, but unlike DH1041 and DH1043. DH1047 also showed reactivity with the SARS-COV-1 S protein. From the panel of NTD-directed antibodies, we selected one infection-enhancing NTD antibody. DH1052, and one neutralizing NTD antibody. DH1050.1, for higher resolution structural determination by cryo-EM. The stabilized SARS-CoV-2 S ectodomain “2P” (S-2P) (51) was used for preparing complexes for structural determination.

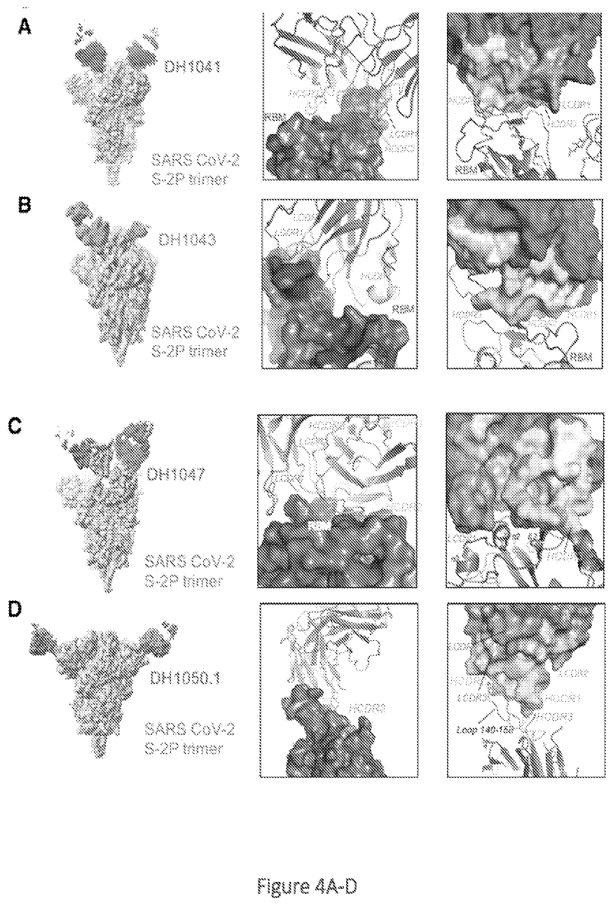

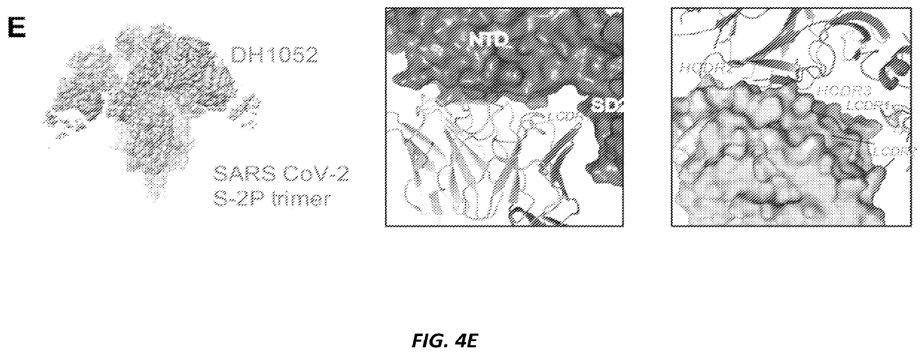

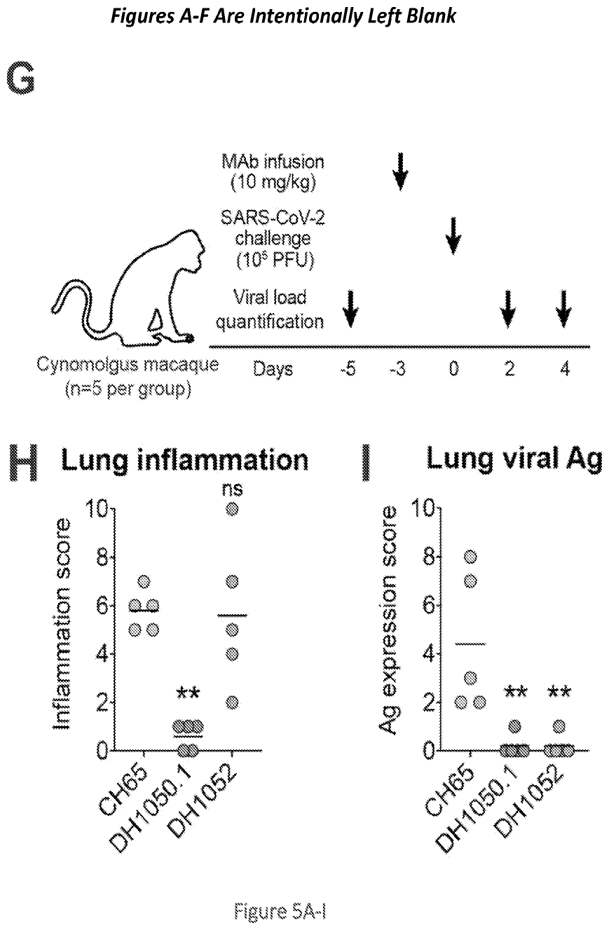

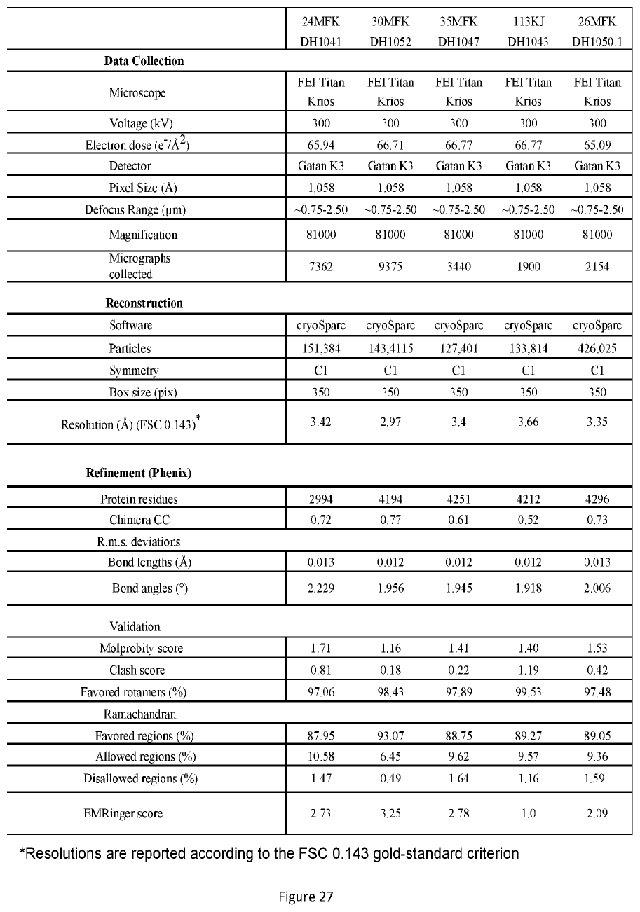

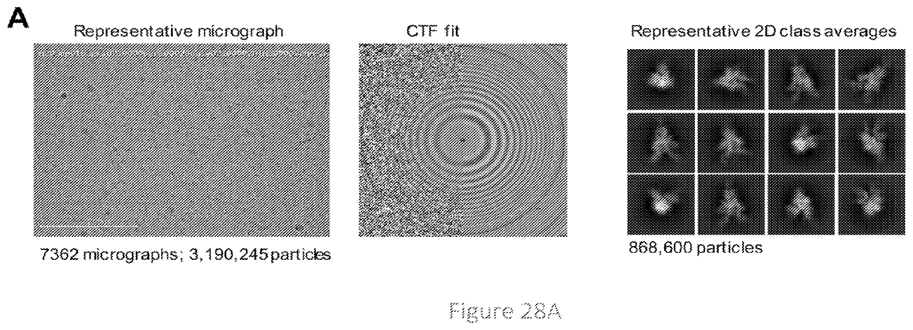

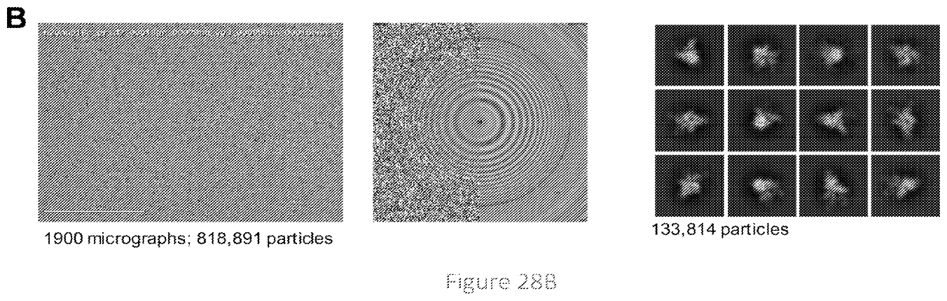

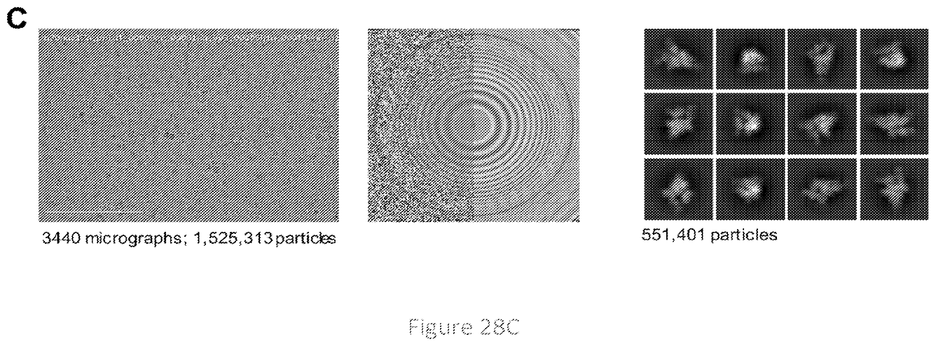

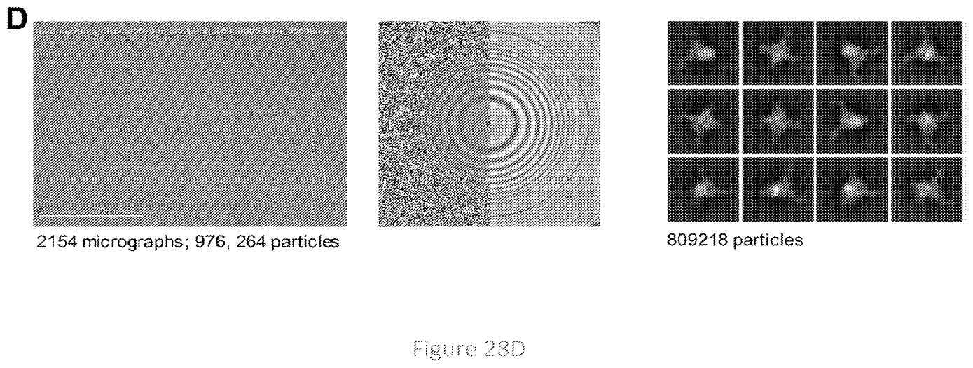

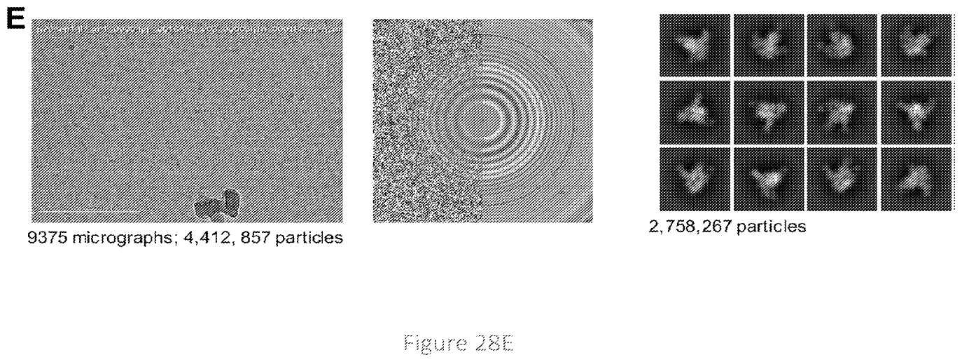

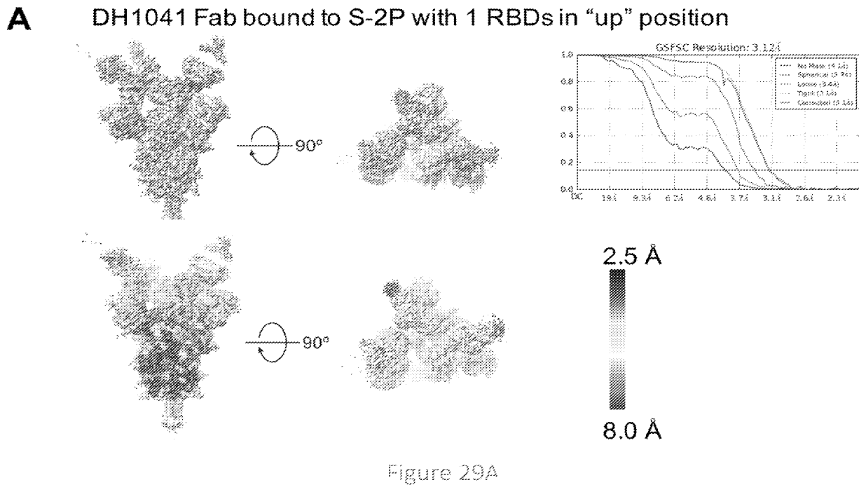

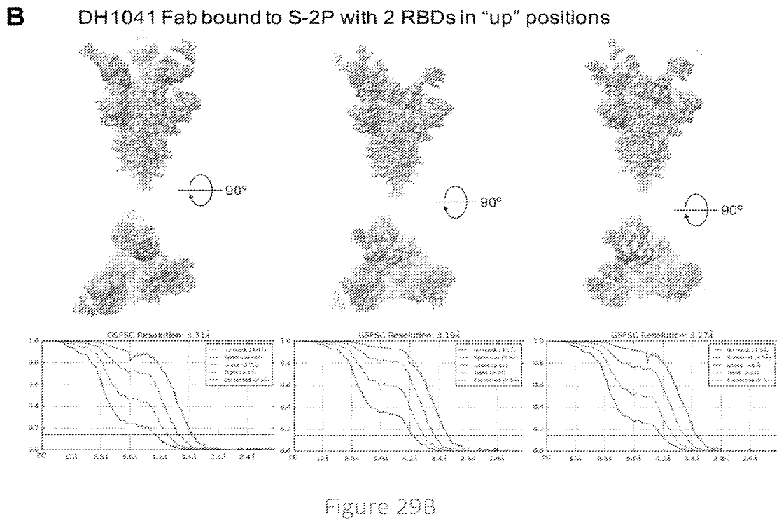

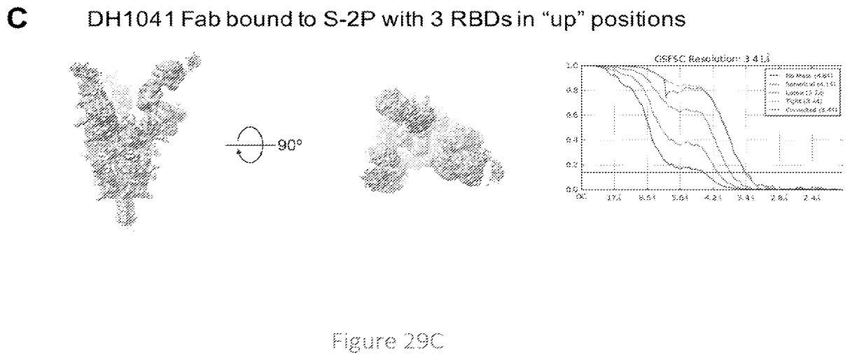

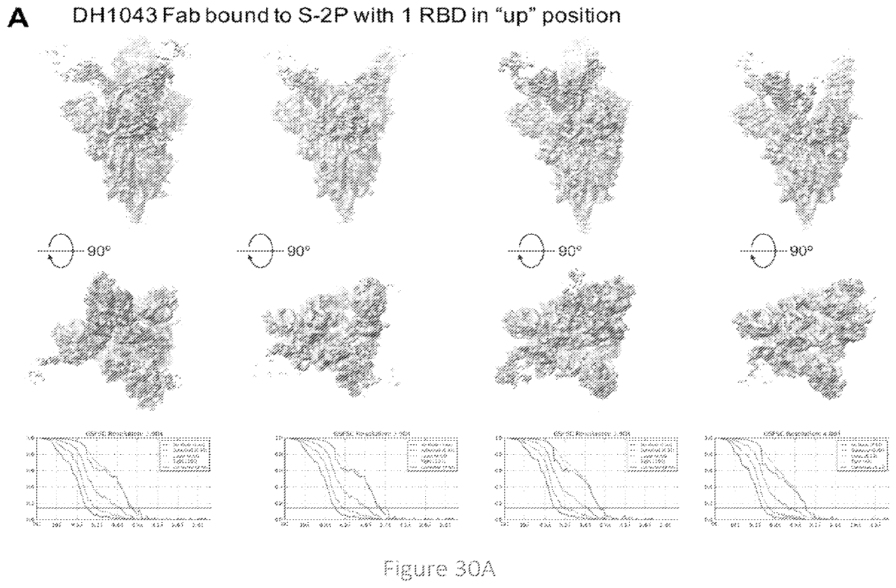

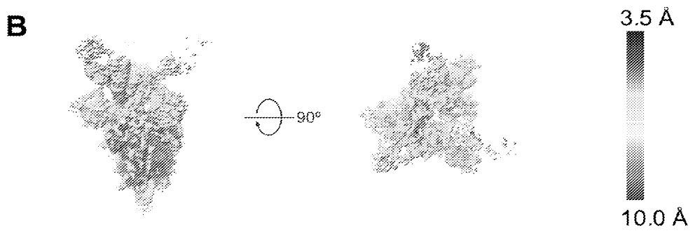

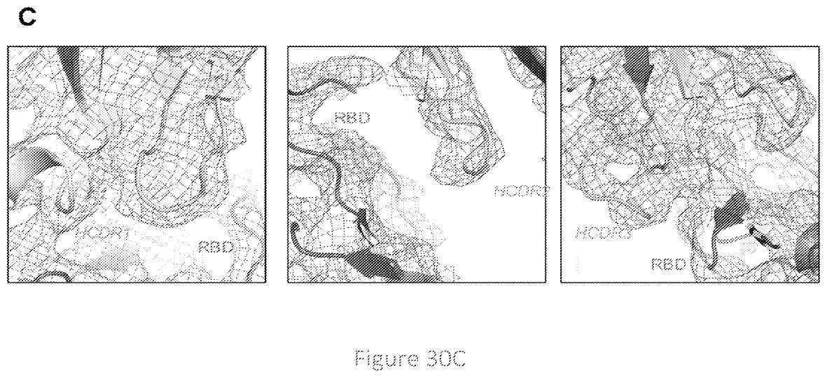

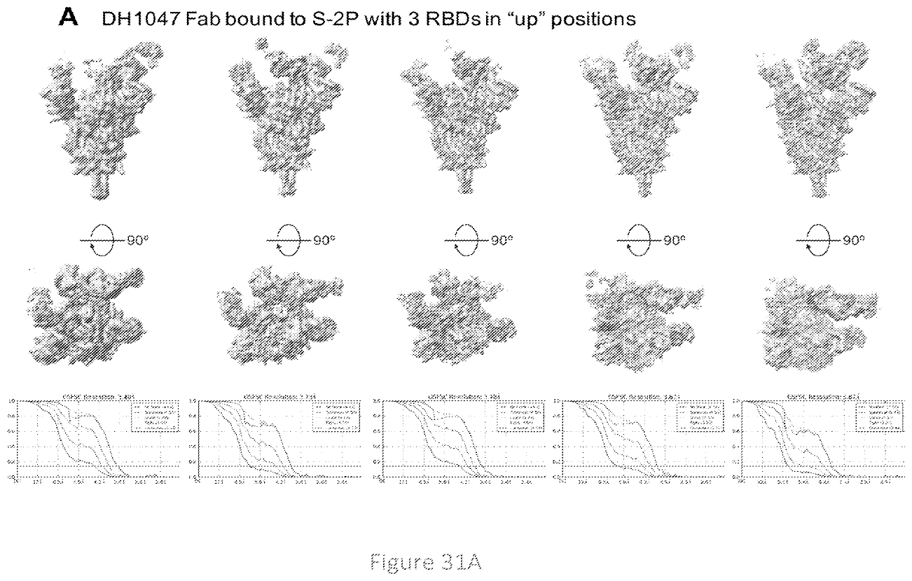

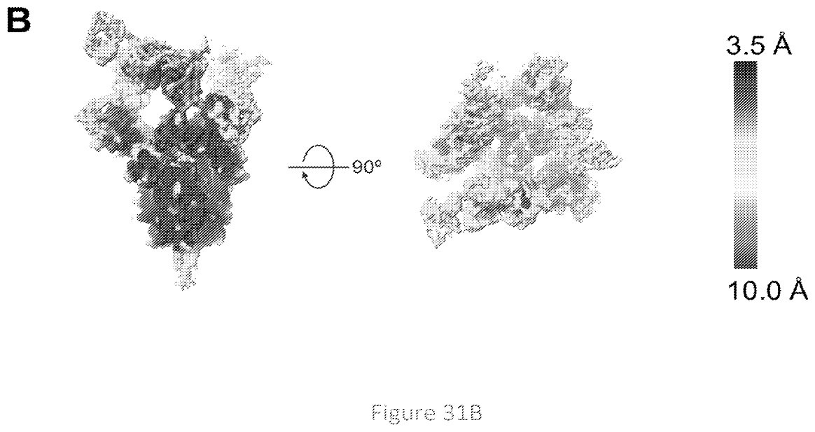

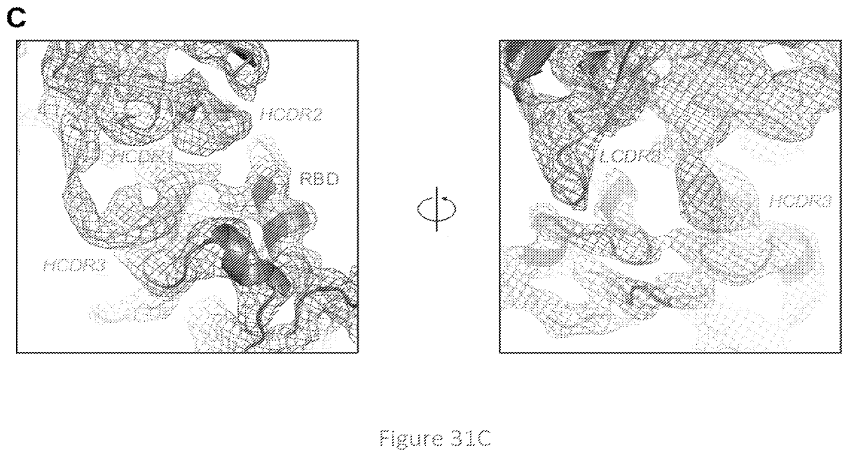

For all three RBD-directed antibodies, the cryo-EM datasets revealed heterogeneous populations of S protein with at least one RBD in the “up” position (FIG. 4, FIGS. 27 and 28). We did not find any unliganded S or any 3-RBD-down S population, even though the unliganded S-2P consistently shows a 1:1 ratio of 1-RBD-up and 3-RBD-down populations (52. 53), indicating that binding of the RBD-directed antibodies to S protein alters RBD dynamics. All S-2P trimers were stoichiometrically bound to 3 Fabs, with antibodies bound to both up and down RBDs in an S-2P trimer.

We observed that the primary epitopes of DH1041 and DH1043 were centered on the Receptor Binding Motif (RBM: residues 483-506) of the RBD (FIG. 4A-B, FIGS. 29 and 30), providing structural basis for the ACE-2 blocking phenotype of these antibodies. While DH1041 utilized its heavy chain complementarity determining regions (CDRs) to contact the RBM, the DH1043 paratope included both its heavy and light chains.

Unlike the RBM-focused epitope of DH1041 and DH1043, the epitope of antibody DH1047 was focused around the a2 and a3 helices and B2 strand that are located outside the N-terminus of the RBM (FIG. 4C, FIG. 31) (54). Contact is also made by RBD residues 500-506 that are also outside the RBM but at its C-terminal end, and stack against the N-terminal end of the a3 helix providing a continuous interactive surface for DH1047 binding. The DH1047 paratope included heavy chain complementarity determining regions HCDR2. HCDR3 and light chain LCDR1 and LCDR3. The HCDR3 stacks against and interacts with the residues in the β2 strand. Interactions with the β2 strand are also mediated by HCDR2. Similar to DH1041 and DH1043, the DH1047 interacted with an “up” RBD conformation from an adjacent protomer although these interactions were not well-characterized due to disorder in that region.

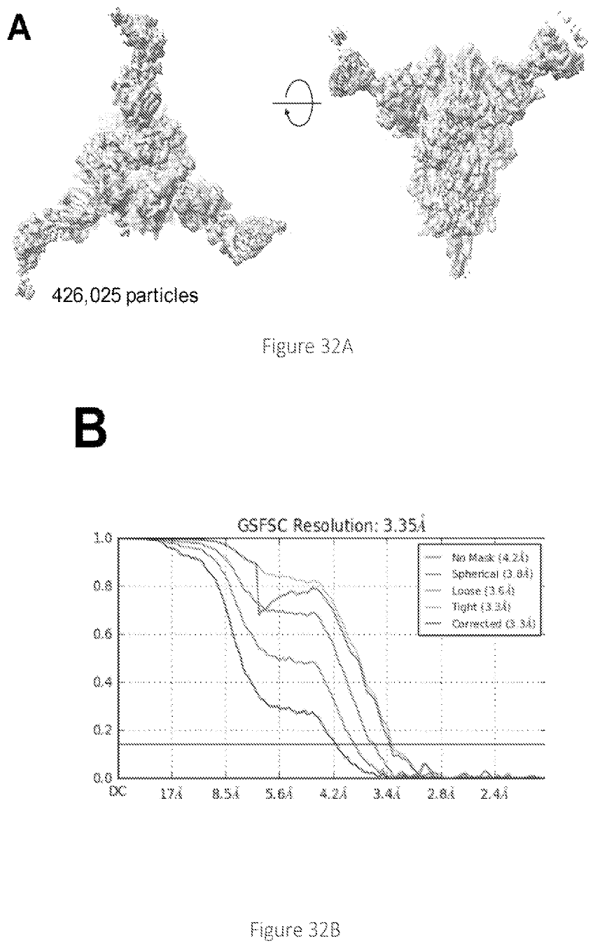

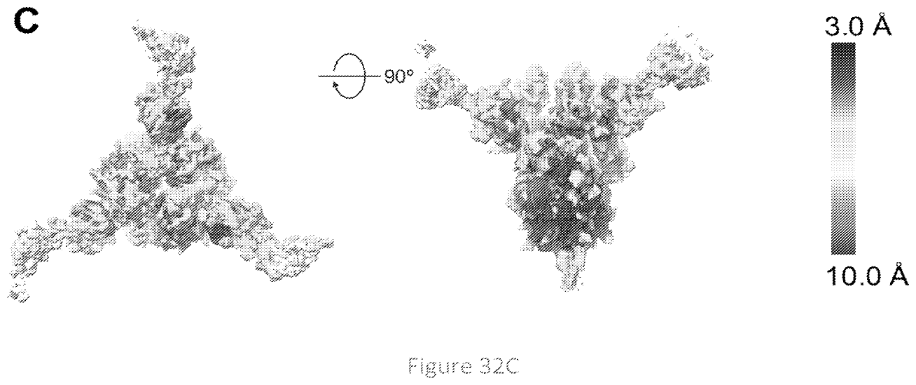

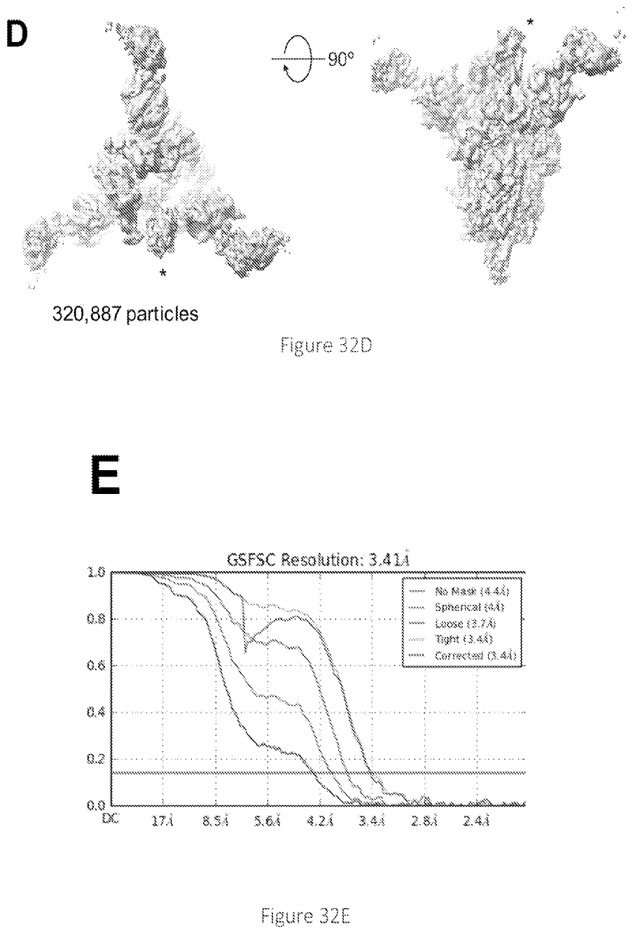

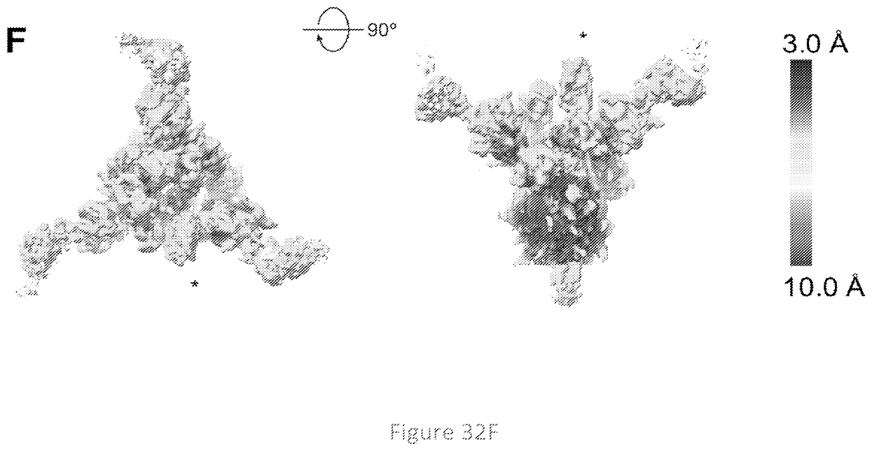

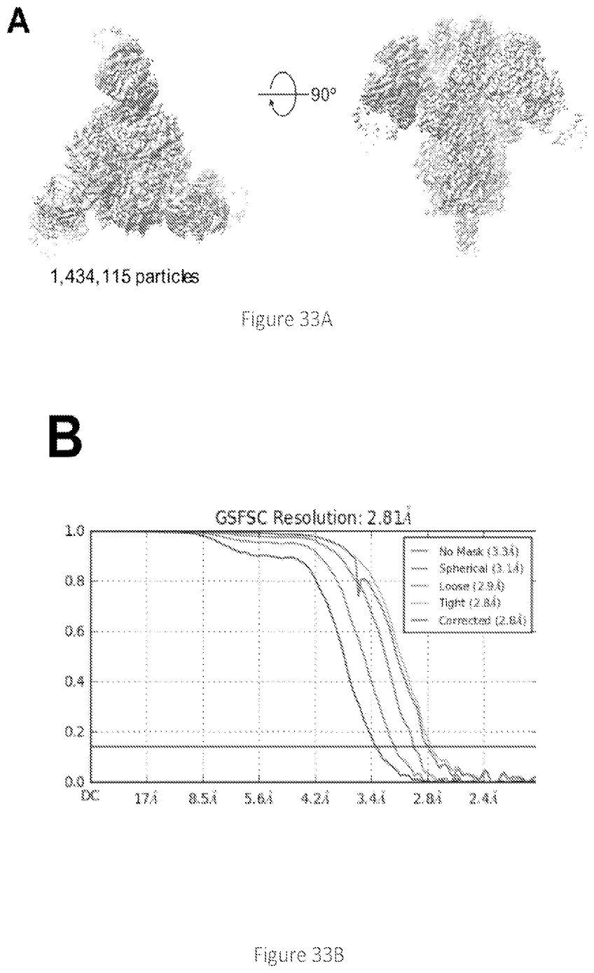

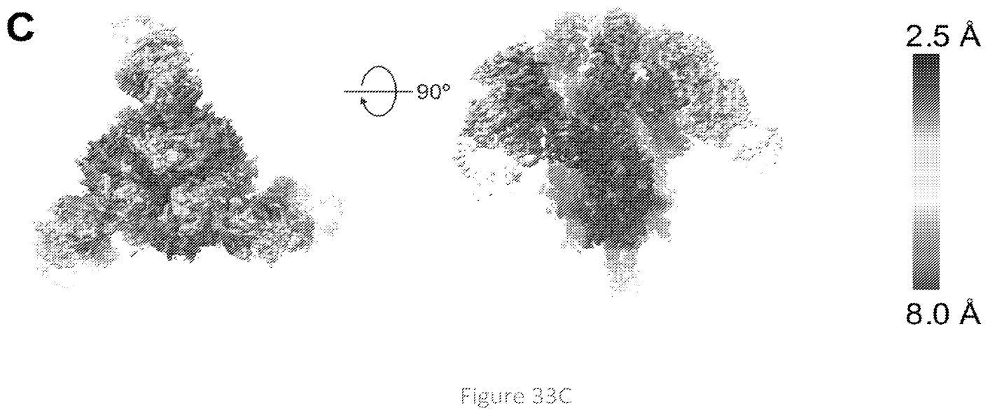

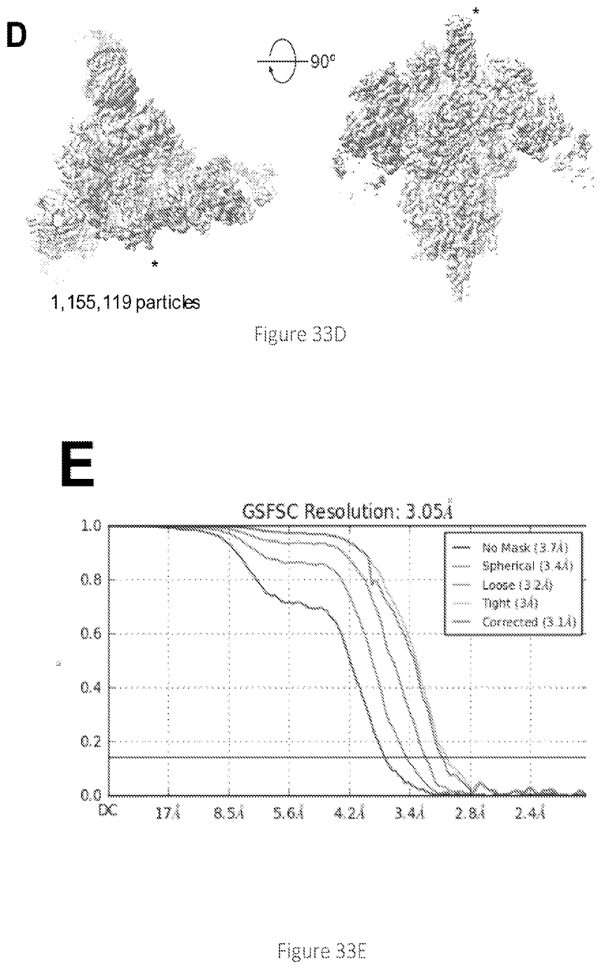

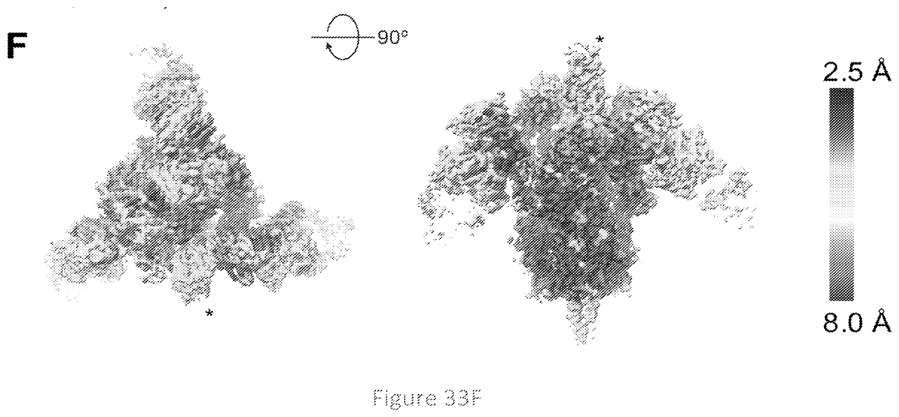

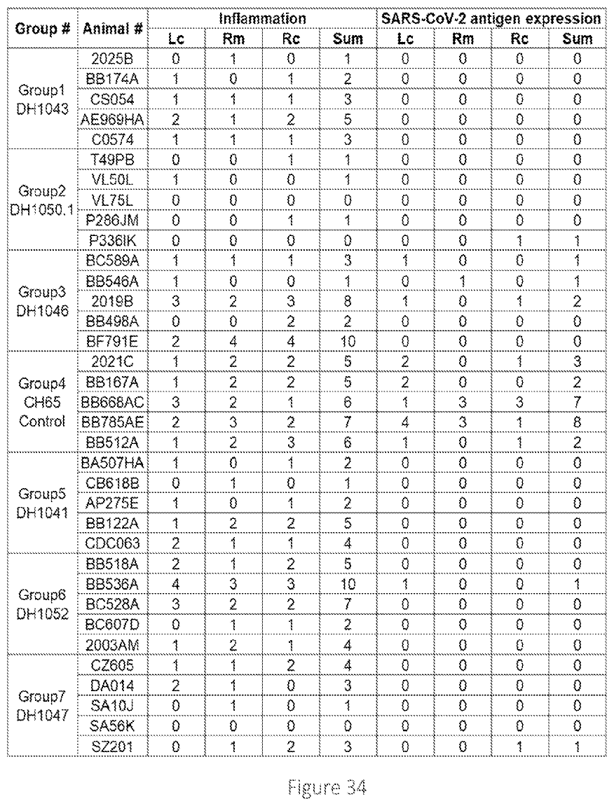

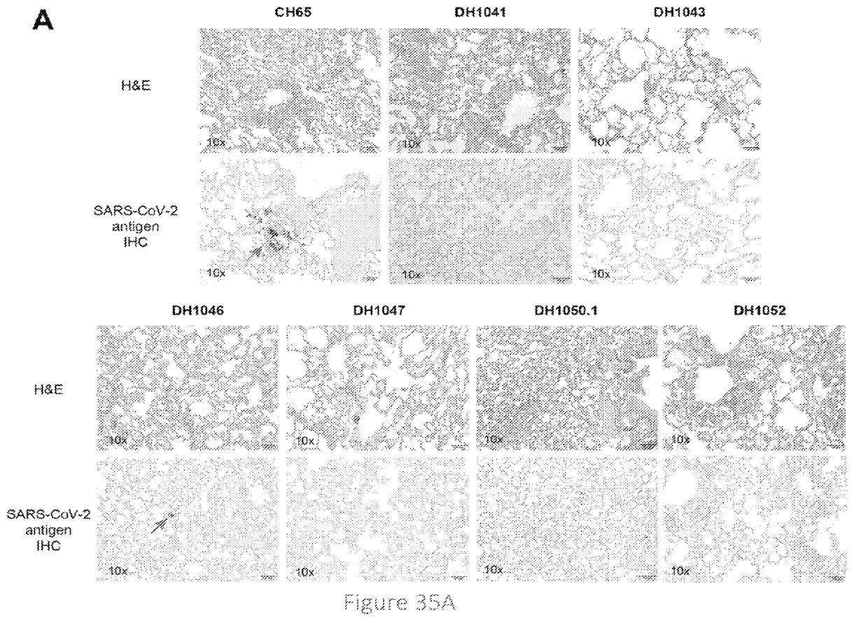

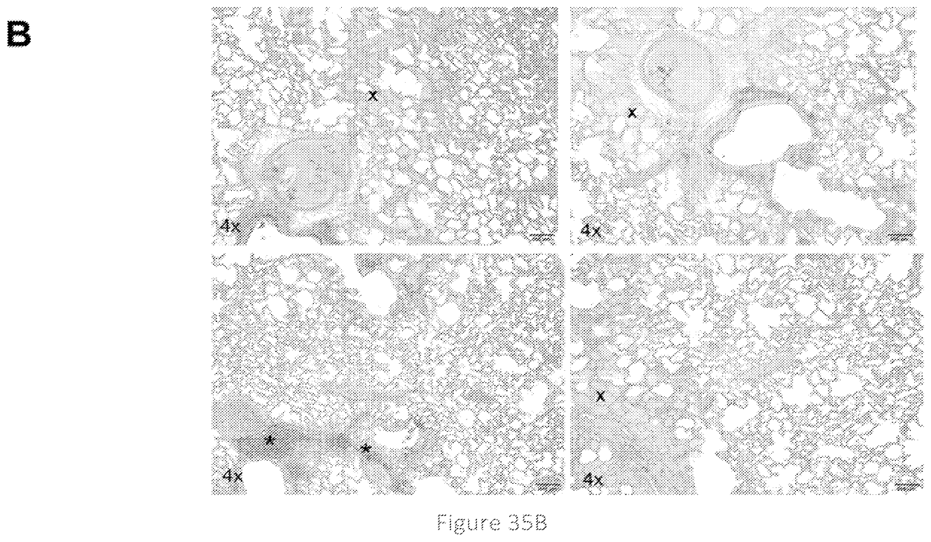

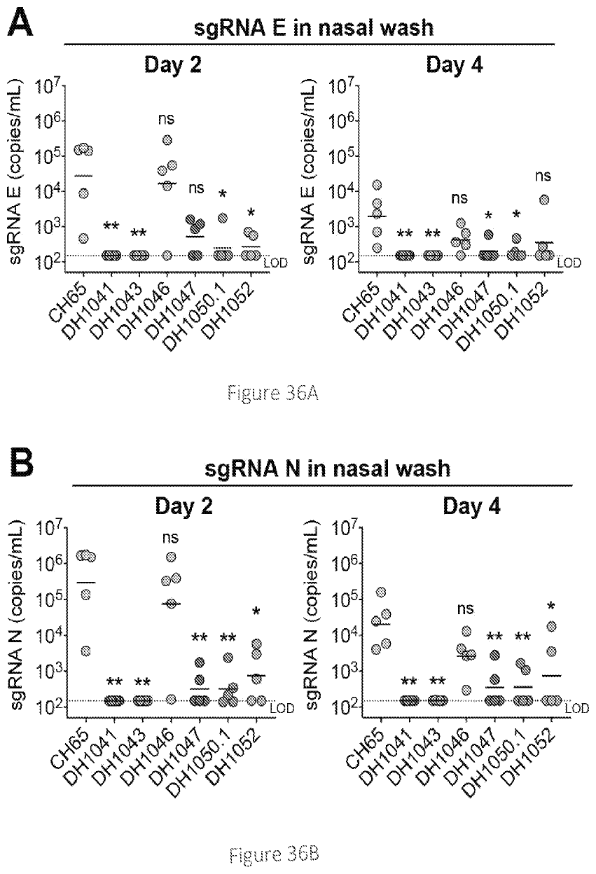

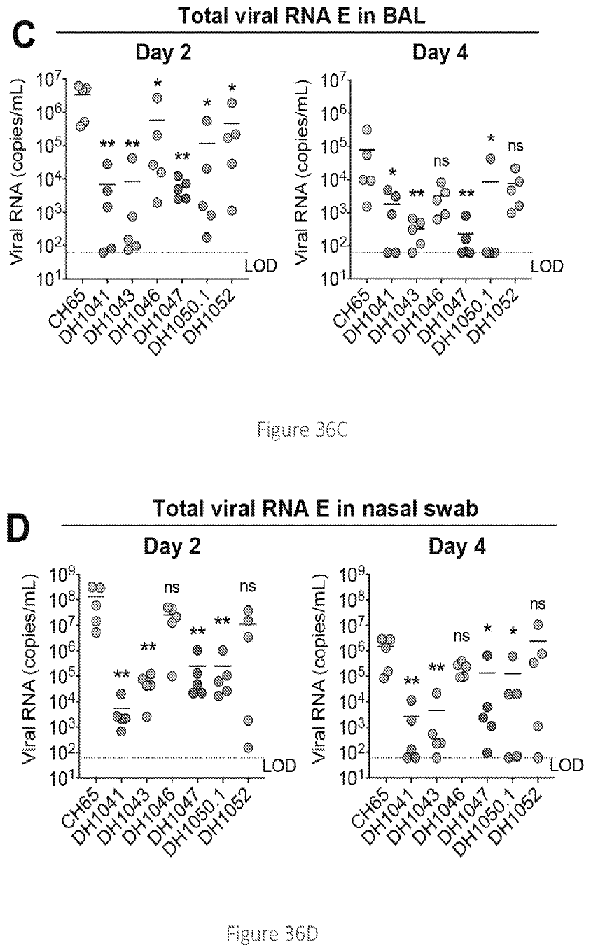

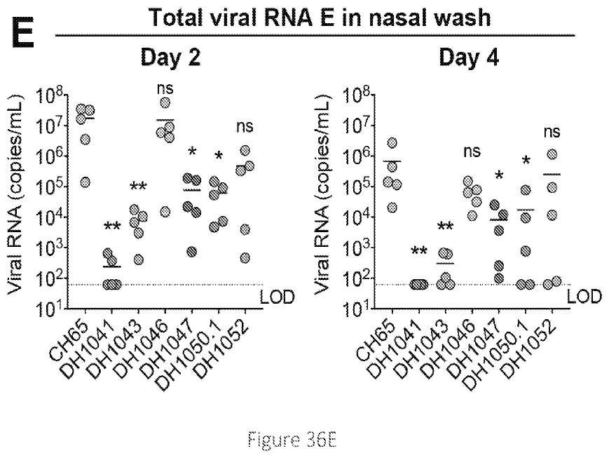

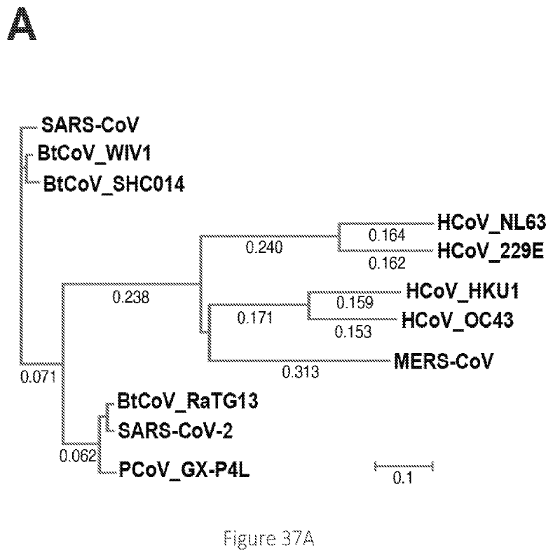

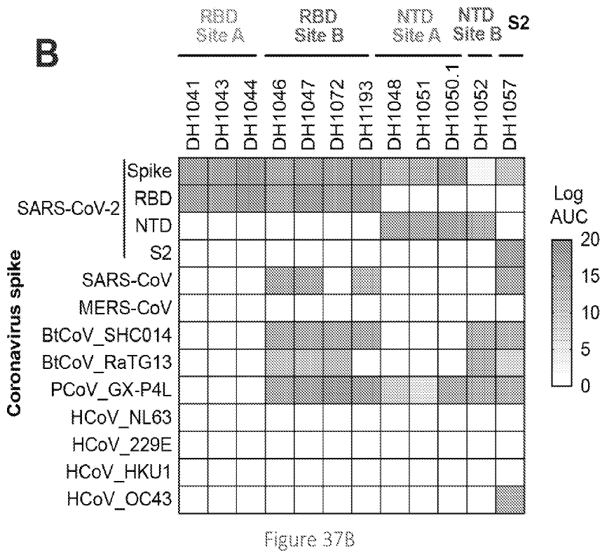

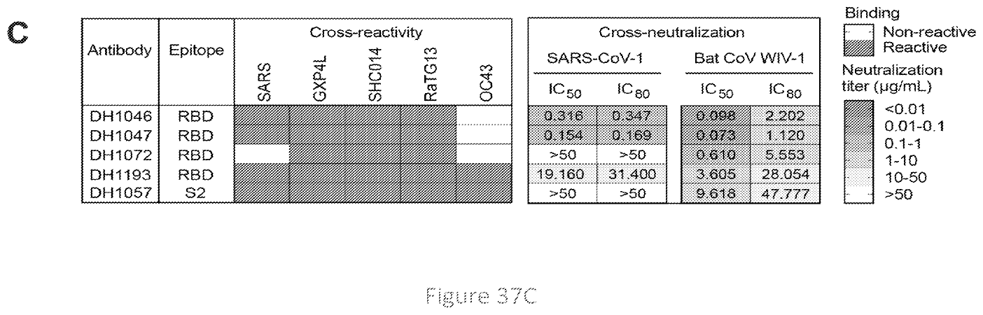

We next determined cryo-EM structures of the NTD-directed neutralizing antibodies. DH1050.1 (FIG. 4D)) and NTD-directed infection-enhancing antibody. DH1052 (FIG. 4E), at 3.4 A and 3.0 A resolutions, respectively. Unlike the RBD-directed antibodies DH1041. DH1043 and DH1047, where we only observed spikes with at least one RBD in the up position, the cryo-EM datasets of DH1050.1—and DH1052-bound complexes showed antibody bound to both 3-RBD-down and 1-RBD-up S-2P spikes (FIGS. 32-33). Consistent with the NSEM reconstructions, the neutralizing antibody DH1050.1 and the non-neutralizing, infection-enhancing antibody DH1052 bound opposite faces of the NTD, with the epitope for the neutralizing antibody DH1050.1 facing the host cell membrane and the epitope for the non-neutralizing, infection-enhancing antibody DH1052 facing the viral membrane. The dominant contribution to the DH1050. 1 epitope came from NTD loop region 140-158 that stacks against the antibody HCDR3 and extends farther into a cleft formed at the interface of the DH1050.1 HCDR1. HCDR2 and HCDR3 loops. The previously described NTD antibody 4A8 interacts with the same epitope in a similar elongated HCDR3-dominated manner making similar contacts, although DH1050.1 and 4A8 (26) show a rotation about the stacked HCDR3 and NTD 140-158 loops, indicating focused recognition of the elongated NTD loop by a class of antibodies sharing the same VH origin. Consistent with their diverse light chain gene origins, the light chains of DH1050.1 and 4A8 do not contact the S protein. The focused recognition of the NTD loop 140-158 by DH1050.1 is reminiscent of the interactions that HIV-1 fusion peptide-directed antibodies make with the HIV-1 Env, where recognition is focused on the conserved region of the flexible fusion peptide (55. 56). In a structurally conserved mechanism, such focused recognition is characterized by robust cryo-EM density at the recognition site but increased disorder away from the site of focused interactions (FIG. 4E, FIG. 33).

The infection enhancing NTD-directed antibody DH1052 bound the NTD at an epitope facing the viral membrane and comprised of residues spanning 27-32. 59-62 and 211-218, with all the CDR loops of both heavy and light chains involved in contacts with the NTD. We also observed contact of the antibody with the glycan at position 603, as well as the conformationally invariant SD2 region.

Thus, we found that the RBD-directed antibodies isolated in this study influenced RBD dynamics and bound only to spike with at least one RBD in the up conformations, and in some cases, also induced the 2-RBD-up and 3-RBD-up spike conformations. In contrast, the NTD-directed antibodies bound to both the 3-RBD-down and 1-RBD-up spikes that are present in the unliganded S-2P. Our data provide a structural explanation for the ACE-2 blocking activity of RBD-directed antibodies as well as for the cross-reactivity of the DH1047 antibody. We observed two distinct orientations for the NTD-directed antibodies that are either neutralizing or non-neutralizing, with the former binding the NTD surface that faces away from the viral membrane and the latter binding the surface that faces the viral membrane.

NTD Antibodies that Mediate SARS-COV-2 Infection Enhancement In Vitro, do not Enhance Infection or Disease In Vivo in Monkeys

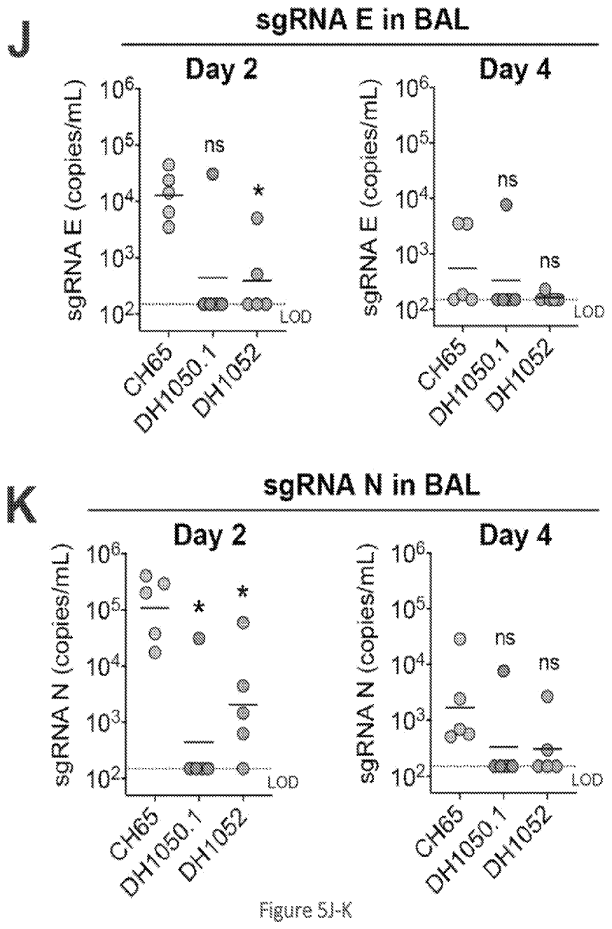

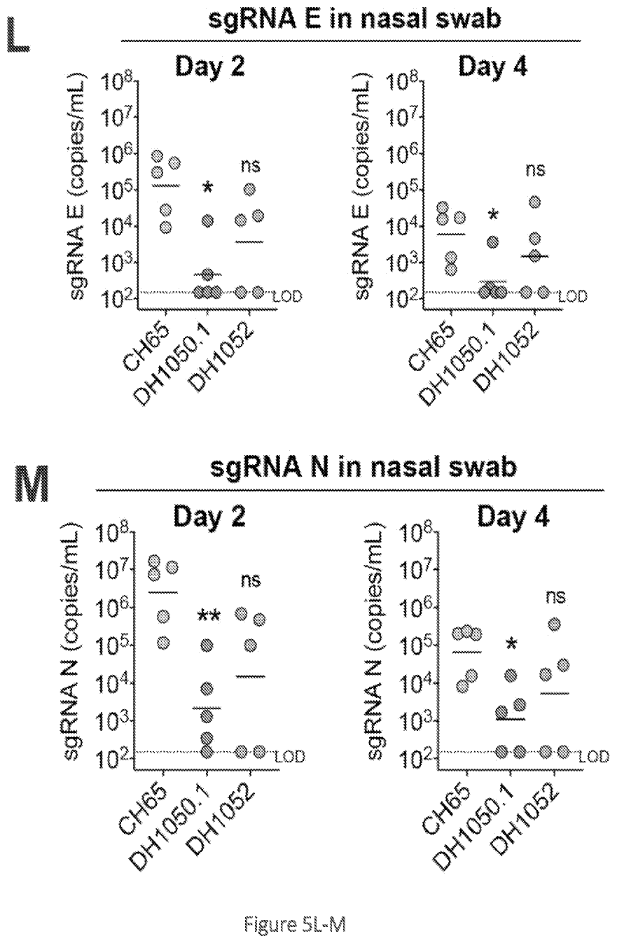

To determine the biological relevance of in vitro infection enhancement by NTD antibodies, we examined the effect of infusion of NTD antibody DH1052 on SARS-COV-2 infection in monkeys (57. 58). Cynomolgus macaques were infused with 10 mg of antibody per kilogram of body weight and then challenged intranasally and intratracheally with 105 plaque forming units of SARS-COV-2 three days later (FIG. 5G) (54). We compared the protective activity of in vitro infection-enhancing antibody DH1052, neutralizing NTD antibody DH1050.1, and negative control influenza antibody CH65 in separate groups of macaques. Overall, macaques that received CH65 and DH1052 had comparable lung inflammation four days after infection (FIG. 5H, FIGS. 34 and 35A). One macaque administered DH1052 showed increased perivascular mononuclear inflammation and perivascular/alveolar edema compared to control antibody-infused animals and compared to the other four animals in the DH1052-treated group (FIG. 35B). Macaques administered DH1050.1 had significantly lower lung inflammation than CH65-infused macaques (FIG. 5H, FIGS. 34 and 35A). Infusion of either neutralizing NTD DH1050.1 or in vitro infection-enhancing antibody DH1052 reduced viral nucleocapsid antigen in the lung (FIG. 51, FIGS. 34 and 35A), envelope (E) gene subgenomic RNA (sgRNA), and nucleocapsid (N) gene sgRNA in the bronchoalveolar fluid (BAL) compared to the same assays in animals treated with negative control antibody (FIGS. 5J-K, FIG. 36C). In nasal swab fluid, macaques showed reduced E and N gene sgRNA when DH1050.1 or DH1052 was infused respectively, with the reduction by DH1050.1 being statistically significant (FIGS. 5L-M, FIGS. 36A-B and 36D-E). Thus, neutralizing NTD antibody DH1050.1 provided protection against SARS-COV-2 infection in cynomolgus macaques while the in vitro infection-enhancing antibody DH1052 showed no infection enhancement but rather showed partial protection from SARS-COV-2 infection.

### In Vitro Infection-Enhancing RBD Antibodies Did not Enhance SARS-COV-2 Infection In Vivo in Nonhuman Primates

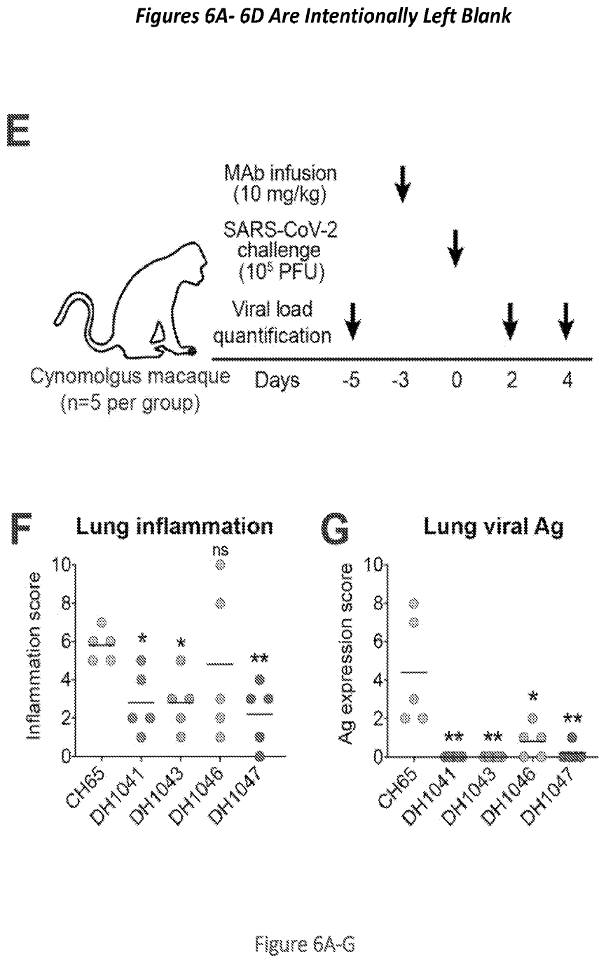

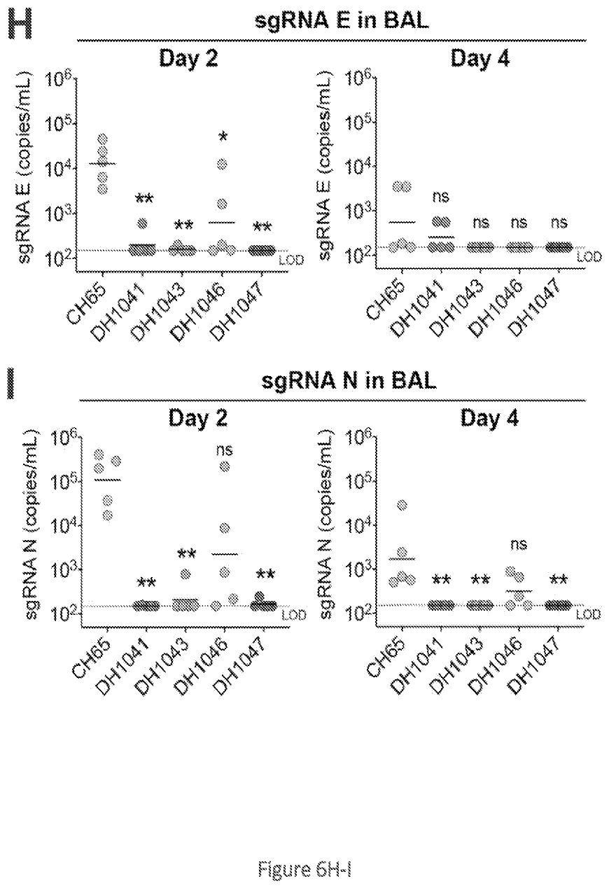

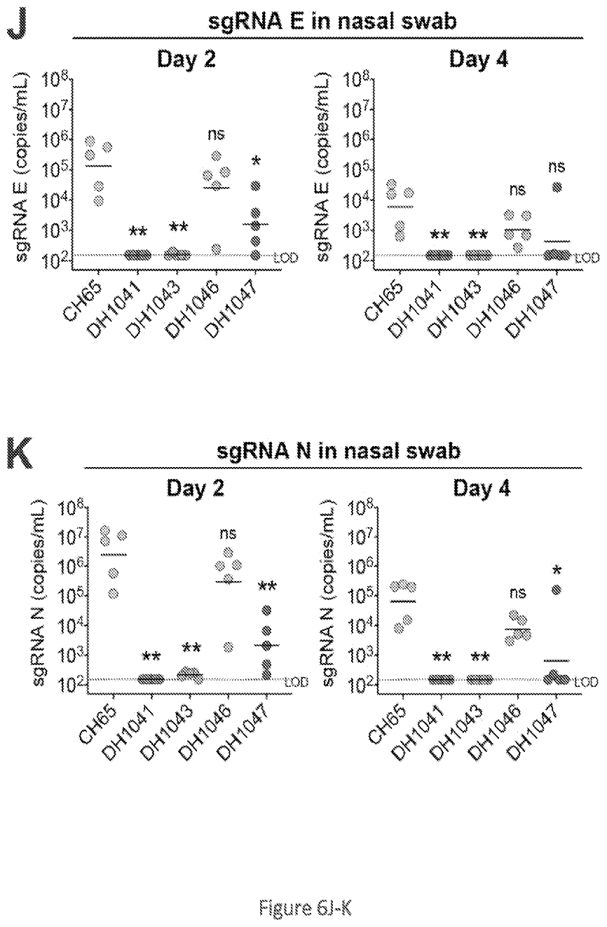

We assessed the in vivo relevance of RBD antibody infection enhancement in the cynomolgus macaque SARS-COV-2 intranasal/intratracheal challenge model. We examined in vivo infection enhancement by RBD antibodies DH1041. DH1043. DH1046, and DH1047 (FIG. 6E). All RBD antibodies that neutralized SARS-COV-2 pseudovirus and live virus assays and enhanced infection in vitro in TZM-bl-FcγRI or—FcγRII cells showed in vivo protection from SARS-COV-2 infection. In macaques administered RBD antibodies DH1041. DH1043, or DH1047, lung inflammation was reduced and lung viral antigen was undetectable in all but one macaque (FIG. 6F-G, FIGS. 34 and 35A). E gene sgRNA and N gene sgRNA were significantly reduced in the upper and lower respiratory tract based on analyses of bronchoalveolar lavage fluid, nasal swabs, and nasal wash samples (FIG. 6H-K, FIG. 36). Monkeys treated with RBD antibody DH1046 exhibited comparable levels of lung inflammation (FIG. 6F-G, FIGS. 34 and 35A) and about 1-log reduction in E gene and N gene sgRNA levels compared to negative control antibody-infused macaques (FIG. 6H-K, FIG. 36). These results further demonstrated that both FcγR-independent and FcγR-dependent in vitro SARS-COV-2 infection enhancement did not translate to SARS-COV-2 in vivo infection enhancement.

### DISCUSSION

Antibody enhancement of virus infection in vitro has been reported for MERS-COV and SARS-COV (24. 33. 38. 59) and is a question facing the safe administration of antibody prophylaxis, antibody therapy, and antibody-based COVID-19 vaccines (60-63). The effects of SARS-COV-2 antibodies have been further questioned since individuals who seroconverted earlier tend to have more severe COVID-19 disease than later seroconverters (64). Here, we demonstrated that SARS-COV-2 antibodies can mediate infection enhancement in vitro, but this in vitro phenotype does not translate to enhanced infection in non-human primate models of SARS-COV-2. We assessed in vivo infection enhancement as severe lung immunopathology or increased respiratory tract virus replication. Neither of these hallmarks of infection enhancement was significantly present in nonhuman primates administered RBD or NTD antibodies that mediate infection enhancement in vitro. Thus, the infection enhancing assays in either 293T cells or in TZM-bl-FcR-expressing cells did not predict in vivo antibody functions. These preclinical results indicate that SARS-COV-2 antibody treatments or the induction of SARS-COV-2 antibodies by vaccination have a low likelihood of exacerbating COVID-19 disease in humans.

Previous studies with polyclonal serum antibodies against SARS-COV have also shown in vitro FcγR-dependent infection enhancement, but no in vivo infection enhancement in hamsters (32). For the present study, there are two explanations for why there is a lack of congruence between in vitro infection enhancement assays and outcomes of passive antibody infusion/SARS-COV-2 challenge studies. First, macrophages and other phagocytes are the target cells that uptake MERS-COV when infection enhancement occurs (33. 65. 66). In contrast, SARS-COV and SARS-COV-2 do not productively infect macrophages (36. 39. 66). Thus, while RBD antibodies can be able to mediate FcγR-dependent virus uptake of SARS-CoV-2 in vitro, in vivo FcγR-dependent virus uptake of SARS-COV-2 may largely lead to abortive infection in macrophages (36. 67).

Second, in vivo SARS-COV-2 antibodies can have the ability to combat SARS-COV-2 replication through antibody effector functions. While circulating in vivo, antibodies can opsonize infected cells or virions and recruit effector immune cells to kill the virus or the infected cells through Fc-mediated mechanisms. A study in a SARS-COV-2 mouse model of acquisition indicated that Fc effector functions contribute to the protective activity of neutralizing antibodies C104, C002, and C110 (68). Thus, antibody effector functions can contribute to the outcome in vivo, but not be accounted for in SARS-COV-2 neutralization assays in vitro. In support of this, infection-enhancing, non-neutralizing NTD antibody DH1052 reduced infectious virus titers in the lung compared to the negative control antibody. The inability of DH1052 to neutralize in vitro indicates it reduced SARS-COV infection in vivo through FcγR-mediated, non-neutralizing effector functions.

We observed two different types of in vitro infection enhancement. First, RBD antibodies mediated classical antibody-dependent enhancement that required FcγRs for virus uptake (62). Previous studies have demonstrated that uptake of MERS-COV or SARS-COV has mostly been mediated by FcγRIIa on the surface of macrophages (33. 36. 39). In contrast to SARS-COV and MERS-COV infection enhancing antibodies, we identified SARS-COV-2 RBD antibodies utilized FcγRIIb or FcγRI. Thus, different FcγRs can mediate SARS-COV and MERS-COV in vitro infection enhancement compared to SARS-COV-2 in vitro infection enhancement. Second, non-neutralizing NTD antibodies mediated FcγR-independent infection enhancement in two different FcγR-negative, ACE-2-expressing cell types. The mechanism of this in vitro enhancement remains unclear, but can be of antibody modulation of S protein conformation. In NSEM studies, NTD antibodies bound to S in a 3-RBD-down conformation. Whether S protein has different entry kinetics or higher affinity for ACE-2 when liganded to infection-enhancing antibody DH1052 will be a focus of studies.

There are over 100 COVID-19 vaccine candidates under development aiming to halt the spread of SARS-COV-2 infection (69. 70), with three large efficacy trials underway in the United States (71). Many of these vaccine candidates induce SARS-COV-2 antibody responses (70. 72-77). It is important to note that administration of COVID-19 convalescent sera to over 35,000 COVID-19 patients have demonstrated the treatment to be safe and is not associated with enhanced disease (78). Our results indicate while SARS-COV-2 enhancement occurred in SARS-COV-2 infection assays in vitro, these assays did not indicate a risk of infection enhancement in mice or monkeys in vivo. The SARS-COV-2-infected individual from whom many of our antibodies were isolated did not progress to severe COVID-19 disease. However, antibody and vaccine treatments require proper safety evaluation in clinical trials, since antibody-dependent infection enhancement is not the only safety concern of such medical countermeasures. These results indicate that antibody-induced enhancement of infection will not be a biologically relevant adverse effect following COVID-19 vaccination in humans.

### REFERENCES CITED IN THIS EXAMPLE

1. B. S. Graham, Rapid COVID-19 vaccine development. Science 368, 945-946 (2020).
2. G. D. Sempowski, K. O. Saunders, P. Acharya, K. J. Wiehe, B. F. Haynes, Pandemic Preparedness: Developing Vaccines and Therapeutic Antibodies For COVID-19. Cell 181, 1458-1463 (2020).
3. D. Corti, A. Lanzavecchia, Broadly neutralizing antiviral antibodies. Annu Rev Immunol 31, 705-742 (2013).
4. M. B. Battles, J. S. Mclellan, Respiratory syncytial virus entry and how to block it. Nat Rev Microbiol 17, 233-245 (2019).
5. A. Dashti, A. L. DeVico, G. K. Lewis, M. M. Sajadi, Broadly Neutralizing Antibodies against HIV: Back to Blood. Trends Mol Med 25, 228-240 (2019).
6. L. Du, S. Jiang, Immunotherapy of SARS based on combinations of neutralizing human monoclonal antibodies. Future Virol 5, 141-144 (2010).
7. S. Jiang, C. Hillyer, L. Du, Neutralizing Antibodies against SARS-COV-2 and Other Human Coronaviruses. Trends Immuno/41, 355-359 (2020).
8. J. Xu et al., Antibodies and vaccines against Middle East respiratory syndrome coronavirus. Emerg Microbes Infect 8, 841-856 (2019).
9. R. Shi et al., A human neutralizing antibody targets the receptor binding site of SARS-COV-2. Nature, (2020).
10. A. O. Hassan et al., A SARS-COV-2 infection model in mice demonstrates protection by neutralizing antibodies. Cell, (2020).
11. Y. Wu et al., A noncompeting pair of human neutralizing antibodies block COVID-19 virus binding to its receptor ACE2. Science, (2020).
12. T. F. Rogers et al., Isolation of potent SARS-COV-2 neutralizing antibodies and protection from disease in a small animal model. Science, (2020).
13. Y. Cao et al., Potent neutralizing antibodies against SARS-COV-2 identified by high-throughput single-cell sequencing of convalescent patients' B cells. Cell, (2020).
14. X. Chen et al., Human monoclonal antibodies block the binding of SARS-COV-2 spike protein to angiotensin converting enzyme 2 receptor. Cell Mol Immunol 17, 647-649 (2020).
15. B. Ju et al., Human neutralizing antibodies elicited by SARS-COV-2 infection. Nature, (2020).
16. C. Wang et al., A human monoclonal antibody blocking SARS-COV-2 infection. Nat Commun 11, 2251 (2020).
17. D. Wrapp et al., Structural Basis for Potent Neutralization of Betacoronaviruses by Single-Domain Camelid Antibodies. Cell 181, 1004-1015.e1015 (2020).
18. S. J. Zost et al., Rapid isolation and profiling of a diverse panel of human monoclonal antibodies targeting the SARS-COV-2 spike protein, bioRxiv, (2020).
19. P. J. M. Brouwer et al., Potent neutralizing antibodies from COVID-19 patients define multiple targets of vulnerability. Science, (2020).
20. D. F. Robbiani et al., Convergent antibody responses to SARS-COV-2 in convalescent individuals. Nature, (2020).
21. A. Baum et al., Antibody cocktail to SARS-COV-2 spike protein prevents rapid mutational escape seen with individual antibodies. Science, (2020).
22. J. Hansen et al., Studies in humanized mice and convalescent humans yield a SARS-COV-2 antibody cocktail. Science, (2020).
23. S. J. Zost et al., Potently neutralizing human antibodies that block SARS-COV-2 receptor binding and protect animals, bioRxiv, (2020).
24. L. Liu et al., Potent neutralizing antibodies against multiple epitopes on SARS-CoV-2 spike. Nature 584, 450-456 (2020).
25. D. Pinto et al., Cross-neutralization of SARS-COV-2 by a human monoclonal SARS-COV antibody. Nature 583, 290-295 (2020).
26. X. Chi et al., A potent neutralizing human antibody reveals the N-terminal domain of the Spike protein of SARS-COV-2 as a site of vulnerability. (2020).
27. A. Z. Wec et al., Broad neutralization of SARS-related viruses by human monoclonal antibodies. Science, (2020).
28. A. M. Arvin et al., A perspective on potential antibody-dependent enhancement of SARS-COV-2. Nature 584, 353-363 (2020).
29. A. Iwasaki, Y. Yang, The potential danger of suboptimal antibody responses in COVID-19. Nat Rev Immunol 20, 339-341 (2020).
30. S. Ubol, S. B. Halstead, How innate immune mechanisms contribute to antibody-enhanced viral infections. Clin Vaccine Immunol 17, 1829-1835 (2010).
31. M. Jaume et al., Anti-severe acute respiratory syndrome coronavirus spike antibodies trigger infection of human immune cells via a pH- and cysteine protease-independent FcgammaR pathway. J Virol 85, 10582-10597 (2011).
32. Y. W. Kam et al., Antibodies against trimeric S glycoprotein protect hamsters against SARS-COV challenge despite their capacity to mediate FcgammaRII-dependent entry into B cells in vitro. Vaccine 25, 729-740 (2007).
33. Y. Wan et al., Molecular Mechanism for Antibody-Dependent Enhancement of Coronavirus Entry. J Virol 94, (2020).
34. S. F. Wang et al., Antibody-dependent SARS coronavirus infection is mediated by antibodies against spike proteins. Biochem Biophys Res Commun 451, 208-214 (2014).
35. M. Yilla et al., SARS-coronavirus replication in human peripheral monocytes/macrophages. Virus Res 107, 93-101 (2005).
36. M. S. Yip et al., Antibody-dependent enhancement of SARS coronavirus infection and its role in the pathogenesis of SARS. Hong Kong Med J 22, 25-31 (2016).
37. M. S. Yip et al., Antibody-dependent infection of human macrophages by severe acute respiratory syndrome coronavirus. Virol J 11, 82 (2014).
38. Q. Wang et al., Immunodominant SARS Coronavirus Epitopes in Humans Elicited both Enhancing and Neutralizing Effects on Infection in Non-human Primates. ACS Infect Dis 2, 361-376 (2016).
39. S. Bournazos, A. Gupta, J. V. Ravetch, The role of IgG Fc receptors in antibody-dependent enhancement. Nat Rev Immunol 20, 633-643 (2020).
40. H. X. Liao et al., High-throughput isolation of immunoglobulin genes from single human B cells and expression as monoclonal antibodies. J Virol Methods 158, 171-179 (2009).
41. H. X. Liao et al., Co-evolution of a broadly neutralizing HIV-1 antibody and founder virus. Nature 496, 469-476 (2013).
42. Y. J. Hou et al., SARS-COV-2 Reverse Genetics Reveals a Variable Infection Gradient in the Respiratory Tract. Cell 182, 429-446 e414 (2020).
43. A. Takada, H. Ebihara, H. Feldmann, T. W. Geisbert, Y. Kawaoka, Epitopes required for antibody-dependent enhancement of Ebola virus infection. J Infect Dis 196 Suppl 2, S347-356 (2007).
44. F. Wu et al., Antibody-dependent enhancement (ADE) of SARS-COV-2 infection in recovered COVID-19 patients: studies based on cellular and structural biology analysis, medRxiv, 2020.2010.2008.20209114 (2020).
45. C. L. Hsieh et al., Structure-based design of prefusion-stabilized SARS-COV-2 spikes. Science 369, 1501-1505 (2020).
46. Q. Wang et al., Structural and Functional Basis of SARS-COV-2 Entry by Using Human ACE2. Cell 181, 894-904 e899 (2020).
47. B. Ju et al., Human neutralizing antibodies elicited by SARS-COV-2 infection. Nature 584, 115-+ (2020).
48. J. Huo et al., Neutralizing nanobodies bind SARS-COV-2 spike RBD and block interaction with ACE2. Nat Struct Mol Biol 27, 846-854 (2020).
49. D. Zhou et al., Structural basis for the neutralization of SARS-COV-2 by an antibody from a convalescent patient. Nat Struct Mol Biol 27, 950-958 (2020).
50. J. E. Pak et al., Structural insights into immune recognition of the severe acute respiratory syndrome coronavirus S protein receptor binding domain. J Mol Biol 388, 815-823 (2009).
51. D. Wrapp et al., Cryo-EM structure of the 2019-nCOV spike in the prefusion conformation. Science 367, 1260-1263 (2020).
52. R. Henderson et al., Controlling the SARS-COV-2 spike glycoprotein conformation. Nat Struct Mol Biol 27, 925-933 (2020).
53. A. C. Walls et al., Structure, Function, and Antigenicity of the SARS-COV-2 Spike Glycoprotein. Cell 181, 281-292 e286 (2020).
54. J. Lan et al., Structure of the SARS-COV-2 spike receptor-binding domain bound to the ACE2 receptor. Nature 581, 215-220 (2020).
55. A. S. Dingens et al., Complete functional mapping of infection- and vaccine-elicited antibodies against the fusion peptide of HIV. PLOS Pathog 14, e1007159 (2018).
56. K. Xu et al., Epitope-based vaccine design yields fusion peptide-directed antibodies that neutralize diverse strains of HIV-1. Nat Med 24, 857-867 (2018).
57. B. Rockx et al., Comparative pathogenesis of COVID-19, MERS, and SARS in a nonhuman primate model. Science 368, 1012-1015 (2020).
58. S. R. Leist, A. Schafer, D. R. Martinez, Cell and animal models of SARS-COV-2 pathogenesis and immunity. Dis Model Mech 13, (2020).
59. J. Gu et al., Multiple organ infection and the pathogenesis of SARS. J Exp Med 202, 415-424 (2005).
60. S. B. Halstead, L. Katzelnick, COVID 19 Vaccines: Can we fear ADE? J Infect Dis, (2020).
61. K. Karthik, T. M. A. Senthilkumar, S. Udhayavel, G. D. Raj, Role of antibody-dependent enhancement (ADE) in the virulence of SARS-COV-2 and its mitigation strategies for the development of vaccines and immunotherapies to counter COVID-19. Hum Vaccin Immunother, 1-6 (2020).
62. W. S. Lee, A. K. Wheatley, S. J. Kent, B. J. DeKosky, Antibody-dependent enhancement and SARS-COV-2 vaccines and therapies. Nat Microbiol 5, 1185-1191 (2020).
63. H. Ulrich, M. M. Pillat, A. Tarnok, Dengue Fever, COVID-19 (SARS-COV-2), and Antibody-Dependent Enhancement (ADE): A Perspective. Cytometry A 97, 662-667 (2020).
64. M. S. Ho et al., Neutralizing antibody response and SARS severity. Emerg Infect Dis 11, 1730-1737 (2005).
65. J. Zhou et al., Active replication of Middle East respiratory syndrome coronavirus and aberrant induction of inflammatory cytokines and chemokines in human macrophages: implications for pathogenesis. J Infect Dis 209, 1331-1342 (2014).
66. K. P. Y. Hui et al., Tropism, replication competence, and innate immune responses of the coronavirus SARS-COV-2 in human respiratory tract and conjunctiva: an analysis in ex-vivo and in-vitro cultures. Lancet Respir Med 8, 687-695 (2020).
67. C. Y. Cheung et al., Cytokine responses in severe acute respiratory syndrome coronavirus-infected macrophages in vitro: possible relevance to pathogenesis. J Virol 79, 7819-7826 (2005).
68. A. Schafer et al., Antibody potency, effector function and combinations in protection from SARS-COV-2 infection in vivo, bioRxiv, (2020).
69. G. Dagotto, J. Yu, D. H. Barouch, Approaches and Challenges in SARS-COV-2 Vaccine Development. Cell Host Microbe 28, 364-370 (2020).
70. J. Wang, Y. Peng, H. Xu, Z. Cui, R. O. Williams, 3rd, The COVID-19 Vaccine Race: Challenges and Opportunities in Vaccine Formulation. AAPS PharmSciTech 21, 225 (2020).
71. B. F. Haynes et al., Prospects for a safe COVID-19 vaccine. Sci Transl Med, (2020).
72. E. J. Anderson et al., Safety and Immunogenicity of SARS-COV-2 mRNA-1273 Vaccine in Older Adults. N Engl J Med, (2020).
73. K. S. Corbett et al., SARS-COV-2 mRNA vaccine design enabled by prototype pathogen preparedness. Nature 586, 567-571 (2020).
74. K. S. Corbett et al., Evaluation of the mRNA-1273 Vaccine against SARS-CoV-2 in Nonhuman Primates. N Engl J Med 383, 1544-1555 (2020).
75. L. A. Jackson et al., An mRNA Vaccine against SARS-COV-2-Preliminary Report. N Engl J Med, (2020).
76. P. M. Folegatti et al., Safety and immunogenicity of the ChAd0×1 nCOV-19 vaccine against SARS-COV-2: a preliminary report of a phase ½, single-blind, randomised controlled trial. Lancet 396, 467-478 (2020).
77. M. J. Mulligan et al., Phase I/II study of COVID-19 RNA vaccine BNT162b1 in adults. Nature 586, 589-593 (2020).
78. M. J. Joyner et al., Effect of Convalescent Plasma on Mortality among Hospitalized Patients with COVID-19: Initial Three-Month Experience, medRxiv, 2020.2008.2012.20169359 (2020).

### Materials and Methods

### Human Subjects

Nasopharyngeal swabs and peripheral blood samples were collected from a convalescent COVID-19 donor (MESSI ID #450905) on designated days after reported onset of COVID symptoms. The SARS-COV-1 donor PBMC were provided by NIH/VRC. Human subject specimens were collected and used with the informed consent of study participants and in compliance with the Duke University Medical Center Institutional Review Board (DUHS IRB Pro00100241).

### Symptom Data Collections

Participant self-reported symptoms were recorded at each time-point for 39 symptom categories (nasal discharge, nasal congestion, sneezing, coughing, shortness of breath, malaise, throat discomfort, fever, headache, shaking chills, loss of smell, loss of taste, excessive sweating, dizziness, pain behind the eyes, itchy/watery eyes, visual blurring, hearing problems, ear pain, confusion, stiff neck, swollen glands, palpitations, chest pain, pain in joints, muscle soreness, fatigue, loss of appetite, abdominal pain, nausea/vomiting, diarrhea, swelling, itchy skin, rash, skin lesions, unusual bleeding, red fingers or toes, red eyes, other: specify). Each symptom was scored on a scale of 0-4, with 0) indicating not present, 1 mild, 2 moderate, 3 severe, and 4 very severe symptoms. Daily symptom count (number of non-zero symptom categories) and symptom severity (sum of all symptom scores) were determined for each survey timepoint. At enrollment, date of symptom onset was determined, and an initial “historical” symptom survey recorded maximum score for each symptom category between symptom onset and study enrollment.

### Expression of Recombinant Viral Proteins

The SARS-COV-2 ectodomain constructs were produce and purified as describe previously (Wrapp, D. et al. 2020). Plasmids encoding Spike-2P and HexaPro (1) were transiently transfected in FreeStyle 293 cells (Thermo Fisher) using Turbo293 (SpeedBiosystems). The cultures were collected on Day 6 post transfection. The cells were separated from the medium by centrifugation. Protein were purified from filtered cell supernatants by StrepTactin resin (IBA) and additionally by size exclusive chromatography using Superose 6 10/300 increase column (GE Healthcare) in 2 mM Tris pH 8, 200mMnNaCl, 0).02% NaN3. SARS-COV-2 NTD was produced as previously described (2). SARS-COV-1 RBD and MERS-COV Spike RBD were cloned into pVRC vector for mammalian expression (FreeStyle 293F or Expi293F suspension cells). The construct contains an HRV 3C-cleavable C-terminal SBP-8xHis tag. Supernatants were harvested 5 days post-transfection and passaged directly over Cobalt-TALON resin (Takara) followed by size exclusion chromatography on Superdex 200 Increase (GE Healthcare) in 1× PBS. Yields from FreeStyle 293F cells can be approximately 50 mg/liter culture. Affinity tags can be removed using HRV 3C protease (ThermoScientific) and the protein repurified using Cobalt-TALON resin to remove the protease, tag and non-cleaved protein.

### Antigen-Specific Single B Cell Sorting

Plasmablasts were sorted by flow cytometry from the SARS-COV-2 donor on Day 11 and Day 15 post symptom onset. PBMCs were stained with optimal concentrations of the following fluorochrome-antibody conjugates: IgD PE (Clone #IA6-2, BD Biosciences, Catalog #555779), CD3 PE-Cy5 (Clone #HIT3a, BD Biosciences, Catalog #555341), CD10 PE-CF594 (Clone #HI10A, BD Biosciences, Catalog #562396), CD27 PE-Cy7 (Clone #0323, eBioscience, Catalog #25-0279), CD38 APC-Alexa Fluor (AF) 700 (Clone #LS198-4-2, Beckman Coulter, Catalog #B23489), CD19 APC-Cy7 (Clone #LJ25C1, BD Biosciences, Catalog #561743), CD16 BV570) (Clone #3G8, Biolegend, Catalog #302035), CD14 BV605 (Clone #M5E2, Biolegend, Catalog #301834), and CD20 BV650 (Clone #2H7, BD, Catalog #563780). The cells were then labeled with Fixable Aqua Live/Dead Cell Stain Kit (Invitrogen. Catalog #L34957). On a BD FACSAria II flow cytometer (BD Biosciences), plasmablasts were identified as viable CD14/CD16/CD19+/CD2010W/IgD/CD27^{high}/CD38^{high} cells and sorted as single cells into 96-well plates containing lysis buffer. Sorted plates were frozen at −80° C., in the DHVI Flow Facility under BSL3 precautions in the Duke Regional Biocontainment Laboratory (Durham. NC) until processing.

Antigen-specific memory B cells (MBCs) were isolated by flow cytometric sorting from the SARS-COV-2 donor on Day 36 post symptom onset, and a donor with SARS-COV-1 history. PBMCs were stained with IgD FITC (Clone #IA6-2. BD Biosciences. Catalog #555778). IgM PerCp-Cy5.5 (Clone #G20-127. BD Biosciences. Catalog #561285). CD10 PE-CF594 (Clone #HI10A. BD Biosciences. Catalog #562396). CD3 PE-Cy5 (Clone #HIT3a. BD Biosciences. Catalog #555341). CD235a PE-Cy5 (Clone #GA-R2. BD Biosciences, Catalog #559944). CD27 PE-Cy7 (Clone #0323, eBioscience. Catalog #25-0279). CD38 APC-AF700) (Clone #LS198-4-2. Beckman Coulter. Catalog #B23489). CD19 APC-Cy7 (Clone #LJ25C1. BD Biosciences. Catalog #561743), CD14 BV605 (Clone #M5E2. Biolegend. Catalog #301834). CD16 BV570 (Clone #3G8. Biolegend. Catalog #302035), and fluorescent-labeled SARS-COV-2 Spike probes (AF647-conjugated Spike-2P. PE-conjugated Spike-2P. AF647-conjugated NTD. AF647-conjugated RBD. VioBright 515-conjugated RBD). The cells were then labeled with Fixable Aqua Live/Dead Cell Stain Kit (Invitrogen. Catalog #L34957). On a BD FACSAria II flow cytometer (BD Biosciences), antigen-specific MBCs were identified as viable CD3/CD14/CD16/CD235a/CD19⁺/IgD/probe⁺ cells and were sorted as single cells into 96-well plates containing lysis buffer. Collection plates were immediately frozen in a dry ice/ethanol bath, and stored at −80° C., in the DHVI Flow Facility under BSL3 precautions in the Duke Regional Biocontiamet Laboratory until processing. Flow cytometric data were analyzed using FlowJo version 10.

### PCR Amplification of Human Antibody Genes

Antibody genes were amplified by RT-PCR from flow cytometry-sorted single B cells using the methods as described previously (3. 4) with modification. The PCR-amplified genes were then purified and sequenced with 10 μM forward and reverse primers. Sequences were analyzed by using the human library in Clonalyst for the VDJ arrangements of the immunoglobulin IGHV. IGKV, and IGLV sequences and mutation frequencies (5). Clonal relatedness of V_{H}D_{H}J_{H} and V_{L}J_{L} sequences was determined as previously described (6).

Expression of Antibody Viable Region Genes as Full-Length IgG Recombinant mAbs

Transient transfection of recombinant antibodies was performed as previously described (4). Briefly, purified PCR products were used for overlapping PCR to generate linear human IgG expression cassettes. The expression cassettes were transfected into 293i cells using ExpiFectamine (Thermo Fisher Scientific. Catalog #A14525). The supernatant samples containing recombinant IgGs were used for IgG quantification and preliminary ELISA binding screening.

The down-selected human antibody genes were then synthesized and cloned (GenScript) in a human IgG1 backbone with 4A mutations to enhance antibody-dependent cell-mediated cytotoxicity (ADCC) or a human IgG1 backbone with a LS mutation to extent antibody half-life (7). Recombinant IgG antibodies were then produced in HEK293i suspension cells by transfection with ExpiFectamine and purified using Protein A resin. The purified IgG antibodies were run in SDS-PAGE for Coomassie blue staining and western blot for quality control and then used for the downstream experiments.

### Antibody Binding ELISA

For ELISA binding assays of Coronavirus Spike antibodies, the antigen panel included SARS-COV-2 Spike S1+S2 ectodomain (ECD) (SINO. Catalog #40589-V08B1). SARS-COV-2 Spike-2P (8). SARS-COV-2 Spike S2 ECD (SINO. Catalog #40590-V08B). SARS-COV-2 Spike RBD from insect cell sf9) (SINO. Catalog #40592-V08B). SARS-COV-2 Spike RBD from mammalian cell 293 (SINO. Catalog #40592-V08H). SARS-COV-2 Spike NTD-Biotin. SARS-COV Spike Protein Delta™ (BEI. Catalog #NR-722). SARS-COV WH20 Spike RBD (SINO. Catalog #40150-V08B2). SARS-COV WH20 Spike SI (SINO, Catalog #40150-V08B1). SARS-COV-1 RBD. MERS-COV Spike S1+S2 (SINO. Catalog #40069-V08B). MERS-COV Spike SI (SINO. Catalog #40069-V08B1). MERS-COV Spike S2 (SINO. Catalog #40070-V08B). MERS-COV Spike RBD (SINO. Catalog #40071-V08B1), MERS-COV Spike RBD. In preliminary ELISA screening of the transient transfection supernatants, we also screened the antibodies against SARS-COV CL Protease protein (BEI. Catalog #30105) and SARS-COV Membrane (M) protein (BEI. Catalog #110705).

For binding ELISA. 384-well ELISA plates were coated with 2 μg/mL of antigens in 0.1 M sodium bicarbonate overnight at 4° C. Plates were washed with PBS+0.05% Tween 20 and blocked with blocked with assay diluent (PBS containing 4% (w/v) whey protein. 15% Normal Goat Serum. 0.5% Tween-20, and 0.05% Sodium Azide) at room temperature for 1 hour. Purified MAb samples in 3-fold serial dilutions in assay diluent starting at 100 μg/mL, or un-diluted transfection supernatant were added and incubated for 1 hour, followed by washing with PBS-0.1% Tween 20. HRP-conjugated goat anti-human IgG secondary Ab (SouthernBiotech, catalog #2040-05) was diluted to 1:10.000 and incubated at room temperature for 1 hour. These plates were washed four times and developed with tetramethylbenzidine substrate (SureBlue Reserve-KPL). The reaction was stopped with 1 M HCl, and optical density at 450 nm (OD450) was determined.

### Affinity Measurements

SPR measurements of SARS-COV-2 antibody Fab binding to Spike-2P or Spike-Hexaro proteins were performed using a Biacore S200 instrument (Cytiva, formerly GE Healthcare. DHVI BIA Core Facility. Durham. NC) in HBS-EP+1× running buffer. The Spike proteins were first captured onto a Series S Streptavidin chip to a level of 300-400 RU for Spike-2P and 350-450) resonance units (RU) for Spike-HexaPro. The antibody Fabs were injected at 0.5 to 500 nM over the captured S proteins using the single cycle kinetics injection mode at a flow rate of 50 uL/min. Association phase was maintained with either 120 or 240 second injections of each Fab at increasing concentrations followed by a dissociation of 600 seconds after the final injection. After dissociation, the S proteins were regenerated from the streptavidin surface using a 30 second pulse of Glycine pH1.5. Results were analyzed using the Biacore S200 Evaluation software (Cytiva). A blank streptavidin surface along with blank buffer binding were used for double reference subtraction to account for non-specific protein binding and signal drift. Subsequent curve fitting analyses were performed using a 1:1 Langmuir model with a local Rmax for the Fabs with the exception of DH1050.1 Fab which was fit using the heterogeneous ligand model with local Rmax. The reported binding curves are representative of two data sets.

### Surface Plasmon Resonance Antibody Blocking Assay

RBD and NTD Abs binding to S protein was measured by surface plasmon resonance (BIAcore 3000; Cytiva, formerly GE Healthcare. DHVI BIA Core Facility. Durham, NC) analysis. Antibody binding competition and blocking were measured by SPR following immobilization by amine coupling of monoclonal antibodies to CM5 sensor chips (BIAcore/Cytiva). Antibody competition experiments were performed by mixing S protein and mAb (30 minutes incubation) followed by injection for 5 minutes at 50 uL/min. In separate assays and from analysis of binding to an identical epitope binding ligand, it was determined that S protein at 20 μm and antibody at 200 μm bind to complete saturation. Antibody blocking assays were performed by co-injecting S protein (20 μM) over mAb immobilized surfaces for 3 minutes at 30 μL/min and a test Ab (200 μM) for 3 minutes at 30 uL/min. The dissociation of the antibody sandwich complex with the spike protein was monitored for 10 minutes with buffer flow and then a 24 second injection of Glycine pH2.0 for regeneration. Blank buffer binding was used for subtraction to account for signal drift. Data analyses were performed with BIA-evaluation 4.1 software (BIAcore/Cytiva).

ACE2-blocking assay

For ACE-2 blocking assays, plates were coated as stated herein with 2 μg/mL recombinant ACE-2 protein, then washed and blocked with 3% BSA in 1×PBS. While assay plates blocked, purified antibodies were diluted as stated herein, only in 1% BSA with 0.05% Tween-20. In a separate dilution plate Spike-2P protein was mixed with the antibodies at a final concentration equal to the EC50 at which spike binds to ACE-2 protein. The mixture was allowed to incubate at room temperature for 1 hour. Blocked assay plates were then washed and the antibody-spike mixture was added to the assay plates for a period of 1 hour at room temperature. Plates were washed and a polyclonal rabbit serum against the same spike protein (nCOV-1 nCOV-2P.293F) was added for 1 hour, washed and detected with goat anti rabbit-HRP (Abcam cat #ab97080) followed by TMB substrate. The extent to which antibodies were able to block the binding spike protein to ACE-2 was determined by comparing the OD of antibody samples at 450 nm to the OD of samples containing spike protein only with no antibody. The following formula was used to calculate percent blocking: blocking %=(100-(OD sample/OD of spike only)*100).

### Negative-Stain Electron Microscopy

For each Fab-spike complex, an aliquot of spike protein at ˜1-5 mg/ml concentration that had been flash frozen and stored at −80° C., was thawed in an aluminum block at 37° C., for 5 minutes: then 1-4 μl of spike was mixed with sufficient Fab to give a 9:1 molar ratio of Fab to spike and incubated for 1 hour at 37° C. The complex was then cross-linked by diluting to a final spike concentration of 0).1 mg/ml into room-temperature buffer containing 150 mM NaCl, 20 mM HEPES pH 7.4, 5% glycerol, and 7.5 mM glutaraldehyde. After 5 minutes cross-linking, excess glutaraldehyde was quenched by adding sufficient 1 M Tris pH 7.4 stock to give a final concentration of 75 mM Tris and incubated for 5 minutes. For negative stain, carbon-coated grids (EMS, CF300-cu-UL) were glow-discharged for 20s at 15 mA, after which a 5-μl drop of quenched sample was incubated on the grid for 10-15 s, blotted, and then stained with 2% uranyl formate. After air drying grids were imaged with a Philips EM420 electron microscope operated at 120 kV, at 82.000× magnification and images captured with a 2k×2k CCD camera at a pixel size of 4.02 Å.

### Image Processing of Negative Stain Images

The RELION 3.0 program was used for all negative stain image processing. Images were imported, CTF-corrected with CTFFIND, and particles were picked using a spike template from previous 2D class averages of spike alone. Extracted particle stacks were subjected to 2-3 rounds of 2D class averaging and selection to discard junk particles and background picks. Cleaned particle stacks were then subjected to 3D classification using a starting model created from a bare spike model. PDB 6vsb, low-pass filtered to 30 Å. Classes that showed clearly-defined Fabs were selected for final refinements followed by automatic filtering and B-factor sharpening with the default Relion post-processing parameters.

### Cryo-EM Sample Preparation, Data Collection and Processing

To prepare antibody-bound complexes of the SARS-COV-2 2P spike, the spike at a final concentration of 1-2 mg/mL, in a buffer containing 2 mM Tris pH 8.0. 200 mM NaCl and 0).02% NaN3, was incubated with 5-6 fold molar excess of the antibody Fab fragments for 30-60 min. 2.5 uL of protein was deposited on a Quantifoil-1.2/1.3 holey carbon grid that had been glow discharged for 15s in a PELCO easiGlow™ Glow Discharge Cleaning System. After a 30 s incubation in >95% humidity, excess protein was blotted away for 2.5 seconds before being plunge frozen into liquid ethane using a Leica EM GP2 plunge freezer (Leica Microsystems). Cryo-EM data were collected on a Titan Krios (Thermo Fisher) equipped with a K3 detector (Gatan). Data were acquired using the Leginon system (9). All the datasets were energy filtered through a either a 20 eV or 30 eV slit. The dose was fractionated over 50 raw frames and collected at 50 ms framerate. Individual frames were aligned and dose-weighted (10). CTF estimation, particle picking. 2D classifications, ab initio model generation, heterogeneous refinements, homogeneous 3D refinements and local resolution calculations were carried out in cryoSPARC (11).

### Cryo-EM Structure Fitting and Analysis

Previously published SARS-COV-2 ectodomain structures of the all ‘down’ state (PDB ID 6VXX) and single RBD ‘up’ state (PDB ID 6VYB), and models of 2-RBD-up and 3-RBD-up states derived from these, were used to fit the cryo-EM maps in Chimera (12). Models of Fabs were generated in SWIS-MODEL and docked into the cryo-EM reconstructions using Chimera. Mutations were made in Coot (13). Coordinates were fit to the maps first using ISOLDE (14) followed by iterative refinement using Phenix (15) real space refinement and subsequent manual coordinate fitting in Coot as needed. Structure and map analysis were performed using PyMol (16). Chimera (12) and ChimeraX (17).

### Live SARS-COV-2 Neutralization Assays

The SARS-COV-2 virus (Isolate USA-WA1/2020. NR-52281) was deposited by the Centers for Disease Control and Prevention and obtained through BEI Resources. NIAID. NIH. SARS-COV-2 Micro-neutralization (MN) assays were adapted from a previous study (18). In short, sera or purified antibodies are diluted two-fold and incubated with 100 TCID₅₀ virus for 1 hour. These dilutions are used as the input material for a TCID₅₀. Each batch of MN includes a known neutralizing control antibody (Clone D001: SINO. CAT #40150-D001). Data are reported as the last concentration at which a test sample protects Vero E6 cells.

SARS-COV-2 Plaque Reduction Neutralization Test (PRNT) were performed in the Duke Regional Biocontainment Laboratory BSL3 (Durham. NC) as previously described with virus-specific modifications (18). Two-fold dilutions of a test sample (e.g, serum, plasma. purified Ab) were incubated with 50 PFU SARS-COV-2 virus (Isolate USA-WA1/2020. NR-52281) for 1 hour. The antibody/virus mixture is used to inoculate Vero E6 cells in a standard plaque assay (19. 20). Briefly, infected cultures are incubated at 37° C. 5% CO₂ for 1 hour. At the end of the incubation. 1 mL of a viscous overlay (1:1 2× DMEM and 1.2% methylcellulose) is added to each well. Plates are incubated for 4 days. After fixation, staining and washing, plates are dried and plaques from each dilution of each sample are counted. Data are reported as the concentration at which 50% of input virus is neutralized. A known neutralizing control antibody is included in each batch run (Clone D001: SINO. CAT #40150-D001). GraphPad Prism was used to determine IC/EC50 values.

### Pseudo-Typed SARS-COV-2 Neutralization Assay and Infection-Enhancing Assays

Neutralization of SARS-COV-2 Spike-pseudotyped virus was performed by adopting an infection assay described previously (22) with lentiviral vectors and infection in either 293T/ACE2.MF (the cell line was kindly provided by Drs. Mike Farzan and Huihui Mu at Scripps). Cells were maintained in DMEM containing 10% FBS and 50 μg/ml gentamicin. An expression plasmid encoding codon-optimized full-length spike of the Wuhan-1 strain (VRC7480), was provided by Drs. Barney Graham and Kizzmekia Corbett at the Vaccine Research Center. National Institutes of Health (USA). The D614G mutation was introduced into VRC7480 by site-directed mutagenesis using the QuikChange Lightning Site-Directed Mutagenesis Kit from Agilent Technologies (Catalog #210518). The mutation was confirmed by full-length spike gene sequencing. Pseudovirions were produced in HEK 293T/17 cells (ATCC cat, no. CRL-11268) by transfection using Fugene 6 (Promega. Catalog #E2692). Pseudovirions for 293T/ACE2 infection were produced by co-transfection with a lentiviral backbone (pCMV AR8.2) and firefly luciferase reporter gene (pHR′ CMV Luc) (23). Culture supernatants from transfections were clarified of cells by low-speed centrifugation and filtration (0.45 μm filter) and stored in 1 ml aliquots at −80° C.

For 293T/ACE2 neutralization assays, a pre-titrated dose of virus was incubated with 8 serial 3-fold or 5-fold dilutions of mAbs in duplicate in a total volume of 150 μl for 1 hr at 37° C., in 96-well flat-bottom poly-L-lysine-coated culture plates (Corning Biocoat). Cells were suspended using TrypLE express enzyme solution (Thermo Fisher Scientific) and immediately added to all wells (10,000 cells in 100 μL of growth medium per well). One set of 8 control wells received cells+virus (virus control) and another set of 8 wells received cells only (background control). After 66-72 hrs of incubation, medium was removed by gentle aspiration and 30 μL of Promega 1× lysis buffer was added to all wells. After a 10 minute incubation at room temperature, 100 μl of Bright-Glo luciferase reagent was added to all wells. After 1-2 minutes, 110 μl of the cell lysate was transferred to a black/white plate (Perkin-Elmer). Luminescence was measured using a PerkinElmer Life Sciences, Model Victor2 luminometer. Neutralization titers are the mAb concentration (IC50/IC80) at which relative luminescence units (RLU) were reduced by 50% and 80% compared to virus control wells after subtraction of background RLUs. Negative neutralization values are indicative of infection-enhancement. Maximum percent inhibition (MPI) is the reduction in RLU at the highest mAb concentration tested.

For the TZM-bl neutralization assays, a pre-titrated dose of virus was incubated with serial 3-fold dilutions of test sample in duplicate in a total volume of 150 μl for 1 hr at 37° C., in 96-well flat-bottom culture plates. Freshly trypsinized cells (10,000 cells in 100 μl of growth medium containing 75 μg/ml DEAE dextran) were added to each well. One set of control wells received cells+virus (virus control) and another set received cells only (background control). After 68-72 hours of incubation, 150 μl of cultured medium was removed from each well, and 100 μl of Britelite Luminescence Reporter Gene Assay System (PerkinElmer Life Sciences) were added and plates incubated for 2 min at room temperature. After this period 150 μl of the lysate were transferred to black solid plates (Costar) for measurements of luminescence in a Perking Elmer instrument. Neutralization titers are the serum dilution at which relative luminescence units (RLU) were reduced by 50% and 80% compared to virus control wells after subtraction of background RLUs. MPI is the reduction in RLU at the highest mAb concentration tested. Infection-enhancing assays were performed with the same format but using TZM-bl cell lines stably expressing each of the four human FcγR receptors (24). In this assay an increase in RLUs over the virus control signal represents FcR-mediated entry.

### Non-Human Primate Protection Study

Groups of five cynomolgus macaques (4-8 kg) were given intravenous infusion with antibodies at 10 mg/kg body weight on Day-3, relative to infectious virus challenge. For each animal, 105 PFU (˜106 TCID₅₀) SARS-COV-2 virus (Isolate USA-WA1/2020) were diluted in 4 mL, and were given by I mL intranasally and 3 mL intratracheally on Day 0. Plasma and serum samples were collected on Day 0. Nasal swabs, nasal washes, and bronchoalveolar lavage (BAL) were collected on Day-5, 2, and 4.

### Histopathology

Lung specimen from nonhuman primates were fixed in 10% neutral buffered formalin, processed, and blocked in paraffin for histological analysis. All samples were sectioned at 5 μm and stained with hematoxylin-eosin (H&E) for routine histopathology. Samples were evaluated by a board-certified veterinary pathologist in a blinded manner. Sections were examined under light microscopy using an Olympus BX51 microscope and photographs were taken using an Olympus DP73 camera.

### Immunohistochemistry (IHC)

Staining for SARS-COV-2 antigen was achieved on the Bond RX automated system with the Polymer Define Detection System (Leica) used per manufacturer's protocol. Tissue sections were dewaxed with Bond Dewaxing Solution (Leica) at 72° C., for 30 min then subsequently rehydrated with graded alcohol washes and Ix Immuno Wash (StatLab). Heat-induced epitope retrieval (HIER) was performed using Epitope Retrieval Solution 1 (Leica), heated to 100° C., for 20 minutes. A peroxide block (Leica) was applied for 5 min to quench endogenous peroxidase activity prior to applying the SARS-COV-2 antibody (1:2000, GeneTex, GTX135357). Antibodies were diluted in Background Reducing Antibody Diluent (Agilent). The tissue was subsequently incubated with an anti-rabbit HRP polymer (Leica) and colorized with 3,3′-Diaminobenzidine (DAB) chromogen for 10 min. Slides were counterstained with hematoxylin.

### Viral RNA Extraction and Quantification

The assay for SARS-COV-2 quantitative Polymerase Chain Reaction (qPCR) detects total RNA using the WHO primer/probe set E_Sarbeco (Charité/Berlin). A QIAsymphony SP (Qiagen, Hilden, Germany) automated sample preparation platform along with a virus/pathogen DSP midi kit and the complex800 protocol were used to extract viral RNA from 800 μL of pooled samples. A reverse primer specific to the envelope gene of SARS-COV-2 (5′-ATA TTG CAG CAG TAC GCA CAC A-3′) was annealed to the extracted RNA and then reverse transcribed into cDNA using SuperScript™ III Reverse Transcriptase (Thermo Fisher Scientific, Waltham, MA) along with RNAse Out (Thermo Fisher Scientific, Waltham, MA). The resulting cDNA was treated with RNase H (Thermo Fisher Scientific, Waltham, MA) and then added to a custom 4× TaqMan™ Gene Expression Master Mix (Thermo Fisher Scientific, Waltham, MA) containing primers and a fluorescently labeled hydrolysis probe specific for the envelope gene of SARS-COV-2 (forward primer 5′-ACA GGT ACG TTA ATA GTT AAT AGC GT-3′, reverse primer 5′-ATA TTG CAG CAG TAC GCA CAC A-3′, probe 5′-6FAM/AC ACT AGC C/ZEN/A TCC TTA CTG CGC TTC G/IABKFQ-3′). The qPCR was carried out on a QuantStudio 3 Real-Time PCR System (Thermo Fisher Scientific, Waltham, MA) using the following thermal cycler parameters: heat to 50° C., hold for 2 min, heat to 95° C., hold for 10 min, then the following parameters are repeated for 50 cycles: heat to 95° C., hold for 15 seconds, cool to 60° C., and hold for 1 minute. SARS-COV-2 RNA copies per reaction were interpolated using quantification cycle data and a serial dilution of a highly characterized custom DNA plasmid containing the SARS-COV-2 envelope gene sequence. Mean RNA copies per milliliter were then calculated by applying the assay dilution factor (DF=11.7). The limit of quantification (LOQ) for this assay is approximately 62 RNA copies per milliliter of sample.

Subgenomic mRNA Assay

SARS-COV-2 E gene and N gene subgenomic mRNA (sgmRNA) was measured by a one-step RT-qPCR adapted from previously described methods (30. 31). To generate standard curves, a SARS-COV-2 E gene sgmRNA sequence, including the 5′UTR leader sequence, transcriptional regulatory sequence (TRS), and the first 228 bp of E gene, was cloned into a pcDNA3.1 plasmid. For generating SARS-COV-2 N gene sgmRNA, the E gene was replaced with the first 227 bp of N gene. The recombinant pcDNA3. 1 plasmids were linearized, transcribed using MEGAscript T7 Transcription Kit (ThermoFisher, Catalog #AM1334), and purified with MEGAclear Transcription Clean-Up Kit (ThermoFisher, Catalog #AM1908). The purified RNA products were quantified on Nanodrop, serial diluted, and aliquoted as E sgmRNA or N sgmRNA standards.

RNA extracted from animal samples or standards were then measured in Taqman custom gene expression assays (ThermoFisher Scientific). For these assays we used TaqMan Fast Virus 1-Step Master Mix (ThermoFisher, catalog #4444432) and custom primers/probes targeting the E gene sgmRNA (forward primer: 5′ CGATCTCTTGTAGATCTGTTCTCE 3″: reverse primer: 5′ ATATTGCAGCAGT ACGCACACA 3″: probe: 5′ FAM-ACACTAGCCATCCTTACTGCGCTTCG-BHQ1 3′) or the N gene sgmRNA (forward primer: 5° C. GATCTCTTGTAGATCTGTTCTC 3″: reverse primer: 5′ GGTGAA CCAAGACGCAGTAT 3″: probe: 5′ FAM-TAACCAGAATGGAGAACGCAGTG GG-BHQ1 3′). RT-Qpcr reactions were carried out on a QuantStudio 3 Real-Time PCR System (Applied Biosystems) or a StepOnePlus Real-Time PCR System (Applied Biosystems) using a program herein: reverse transcription at 50° C., for 5 minutes, initial denaturation at 95° C., for 20 seconds, then 40 cycles of denaturation-annealing-extension at 95° C., for 15 seconds and 60° C. for 30 seconds. Standard curves were used to calculate E or N sgmRNA in copies per ml; the limit of detections (LOD) for both E and N sgRNA assays were 12.5 copies per reaction or 150 copies per mL of BAL/nasal swab/nasal wash.

### Statistics Analysis

Data were plotted using Prism GraphPad 8.0. Wilcoxon rank sum exact test was performed to compare differences between groups with p-value <0.05 considered significant using SAS 9.4 (SAS Institute, Cary, NC).

### REFERENCES

1. C. L. Hsieh et al., Structure-based design of prefusion-stabilized SARS-COV-2 spikes. Science 369, 1501-1505 (2020).
2. T. Zhou et al., Structure-Based Design with Tag-Based Purification and In-Process Biotinylation Enable Streamlined Development of SARS-COV-2 Spike Molecular Probes. Cell Rep, 108322 (2020).
3. J. Wrammert et al., Rapid cloning of high-affinity human monoclonal antibodies against influenza virus. Nature 453, 667-671 (2008).
4. H. X. Liao et al., High-throughput isolation of immunoglobulin genes from single human B cells and expression as monoclonal antibodies. J Virol Methods 158, 171-179 (2009).
5. T. B. Kepler et al., Reconstructing a B-Cell Clonal Lineage. II. Mutation, Selection, and Affinity Maturation. Front Immunol 5, 170 (2014).
6. H. X. Liao et al., Co-evolution of a broadly neutralizing HIV-1 antibody and founder virus. Nature 496, 469-476 (2013).
7. K. O. Saunders, Conceptual Approaches to Modulating Antibody Effector Functions and Circulation Half-Life. Front Immunol 10, 1296 (2019).
8. D. Wrapp et al., Cryo-EM structure of the 2019-nCOV spike in the prefusion conformation. Science 367, 1260-1263 (2020).
9. C. Suloway et al., Automated molecular microscopy: the new Leginon system. J Struct Biol 151, 41-60 (2005).
10. S. Q. Zheng et al., MotionCor2: anisotropic correction of beam-induced motion for improved cryo-electron microscopy. Nat Methods 14, 331-332 (2017).
11. A. Punjani, J. L. Rubinstein, D. J. Fleet, M. A. Brubaker, cryoSPARC: algorithms for rapid unsupervised cryo-EM structure determination. Nat Methods 14, 290-296 (2017).
12. E. F. Pettersen et al., UCSF Chimera-A visualization system for exploratory research and analysis. Journal of Computational Chemistry 25, 1605-1612 (2004).
13. P. Emsley, K. Cowtan, Coot: model-building tools for molecular graphics. Acta Crystallogr D Biol Crystallogr 60, 2126-2132 (2004).
14. T. Croll, ISOLDE: a physically realistic environment for model building into low-resolution electron-density maps. Acta Crystallographica Section D 74, 519-530 (2018).
15. P. V. Afonine et al., Real-space refinement in PHENIX for cryo-EM and crystallography. Acta Crystallographica Section D 74, 531-544 (2018).
16. L. Schrodinger, The PyMOL Molecular Graphics System. (2015).
17. T. D. Goddard et al., UCSF ChimeraX: Meeting modern challenges in visualization and analysis. Protein Sci 27, 14-25 (2018).
18. J. D. Berry et al., Development and characterisation of neutralising monoclonal antibody to the SARS-coronavirus. J Virol Methods 120, 87-96 (2004).
19. C. M. Coleman, M. B. Frieman, Growth and Quantification of MERS-COV Infection. Curr Protoc Microbiol 37, 15E 12 11-19 (2015).
20. J. Kint, H. J. Maier, E. Jagt, Quantification of infectious bronchitis coronavirus by titration in vitro and in ovo. Methods Mol Biol 1282, 89-98 (2015).
21. V. D. Menachery et al., SARS-like WIV1-COV poised for human emergence. Proc Natl Acad Sci U S A 113, 3048-3053 (2016).
22. T. P. Sheahan et al., Broad-spectrum antiviral GS-5734 inhibits both epidemic and zoonotic coronaviruses. Sci Transl Med 9, (2017).
23. Y. J. Hou et al., SARS-COV-2 Reverse Genetics Reveals a Variable Infection Gradient in the Respiratory Tract. Cell 182, 429-446 e414 (2020).
24. B. Korber et al., Tracking Changes in SARS-COV-2 Spike: Evidence that D614G Increases Infectivity of the COVID-19 Virus. Cell 182, 812-827 e819 (2020).
25. L. Naldini, U. Blomer, F. H. Gage, D. Trono, I. M. Verma, Efficient transfer, integration, and sustained long-term expression of the transgene in adult rat brains injected with a lentiviral vector. Proc Natl Acad Sci USA 93, 11382-11388 (1996).
26. L. G. Perez, M. R. Costa, C. A. Todd, B. F. Haynes, D. C. Montefiori, Utilization of immunoglobulin G Fc receptors by human immunodeficiency virus type 1: a specific role for antibodies against the membrane-proximal external region of gp41. J Virol 83, 7397-7410 (2009).
27. K. H. Dinnon, 3rd et al., A mouse-adapted model of SARS-COV-2 to test COVID-19 countermeasures. Nature, (2020).
28. K. H. Dinnon, 3rd et al., A mouse-adapted model of SARS-COV-2 to test COVID-19 countermeasures. Nature, (2020).
29. S. R. Leist et al., A Mouse-Adapted SARS-COV-2 Induces Acute Lung Injury and Mortality in Standard Laboratory Mice. Cell, (2020).
30. R. Wolfel et al., Virological assessment of hospitalized patients with COVID-2019. Nature 581, 465-469 (2020).
31. J. Yu et al., DNA vaccine protection against SARS-COV-2 in rhesus macaques. Science 369, 806-811 (2020).

### Example 6 DH1042-SARS-COV-2 Neutralizing Antibody

### Donor Selection, Antibody Gene Isolation, Sequencing, and Cloning

Donor Selection Isolation: Human antibody gene sequences were isolated from antigen-specific memory phenotype B cells from a convalescent COVID-19 donor using recombinant SARS-COV-2 spike protein hooks as previously described (2). FIG. 47 shows the course of selected donor's (ID #450905) COVID disease. Shown is a self-reported symptom severity score, viral load in nasal swab viral transport medium (qRT-PCR) and serum IgG area against NP and two S protein preparations. Thirty-six days post-symptom onset the selected donor had a serum neutralizing antibody titer of >640 against live SARS-COV-2, using a classic live virus PRNT assay (3). SARS-COV-2 Spike-specific memory B cell from cryopreserved donor PBMC form day 36 post symptom on set were sorted to isolate DH1042. Individual antigen-specific memory B cells were sorted into wells of 96-well plates containing a lysis buffer in preparation for RT-PCR amplification of the human IgG, IgK and IgL genes in each cell.

Sequencing/Cloning: The immunogenetics of DH1042 are detailed in Table 13. For in vitro and in vivo characterization of binding and function DH1042 Vh and VI were cloned respectively into protein expression vectors pH026103 LS.v2 and pK023879.v2 for transient mammalian expression of DH1042 as an IgG1 with the LS Fc modification (M428L/N434S) and an IgK (4). Amino acid sequences of the heavy and light chains are in Table 14. Small- and large-scale mammalian cell (293) expression cultures are used to produce mg quantities of protein which are subsequently protein A purified, quantified and confirmed to contain <2.0 EU/mg of endotoxin. This material is then used for in vitro and in vivo studies.

TABLE 13
DH1042 immunogenetics

| | VH Mut | VL Mut | CDRH3 | CDRL3 | | |
|---|---|---|---|---|---|---|
| Duke Ab ID | % | % | Length | Length | VH | VL |
| DH1042 | 2.0% | 0.7% | 16 | 9 | VH1-69 | VK1-39 |

TABLE 14
DH1042 amino acid sequences
(Vh/Vl are represented by the grey shaded sequences)

>DH1042 Heavy Chain pH026103_LS.v2
MGWSCIILFLVATATGVHS

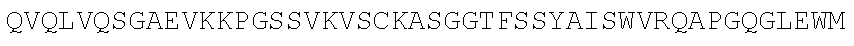

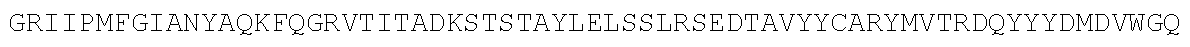

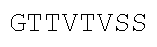

ASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKENWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVLHEALHSHYTQKSLSLS
PGK.
>DH1042 Light Chain pK023879.v2
MGWSCIILFLVATATGVHS

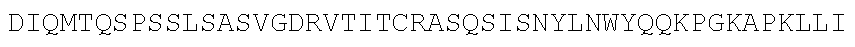

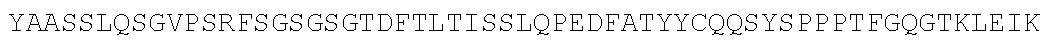

RTVAAPSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC.

### Binding Specificity and Epitope

DH1042 is SARS-COV-2 Spike Receptor Binding Domain (RBD) specific. DH1042 was screened for binding by standard ELISA against a panel of coronavirus antigens from SARS-Cov-2 (Spike, RBD, S2, NTD), SARS-COV-1 (SARS Spike, RBD, S1), MERS-COV (Spike, RBD, S1, S2), seasonal cold coronavirus (CoV-NL63, CoV-229E, CoV-HKUI, CoV-OC43), bat coronavirus (BCoV) and pangolin coronavirus (PCOV). FIG. 48 shows the area under the curve for DH1042 binding and positive control antibodies or anti-sera.

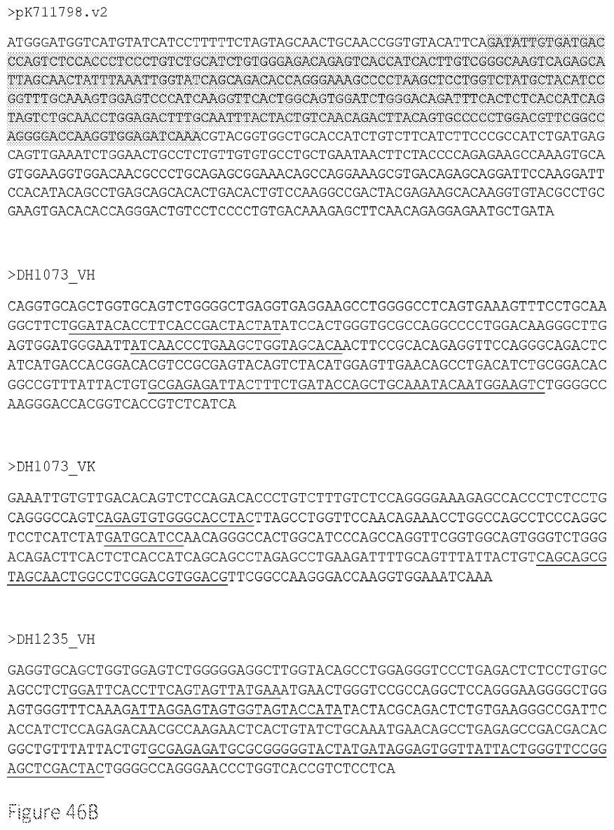

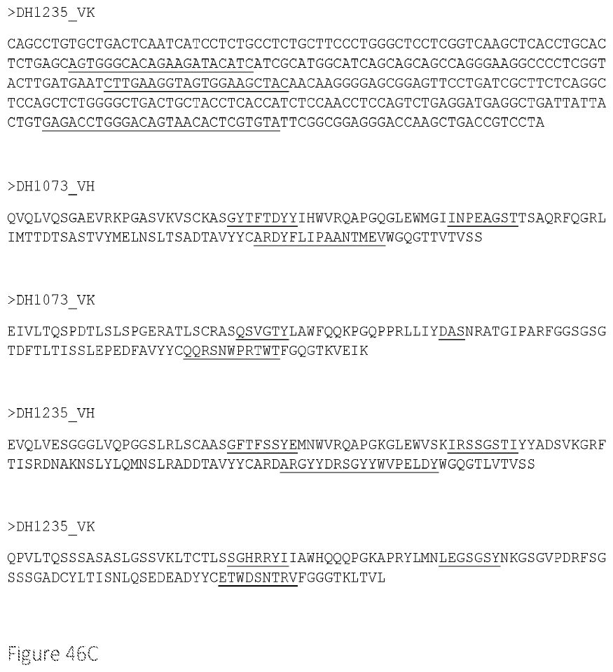

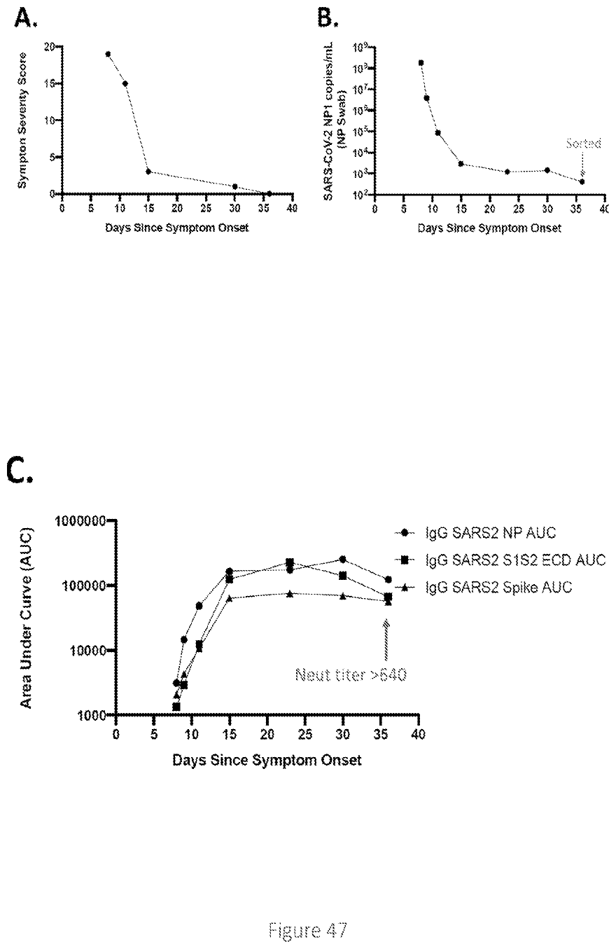

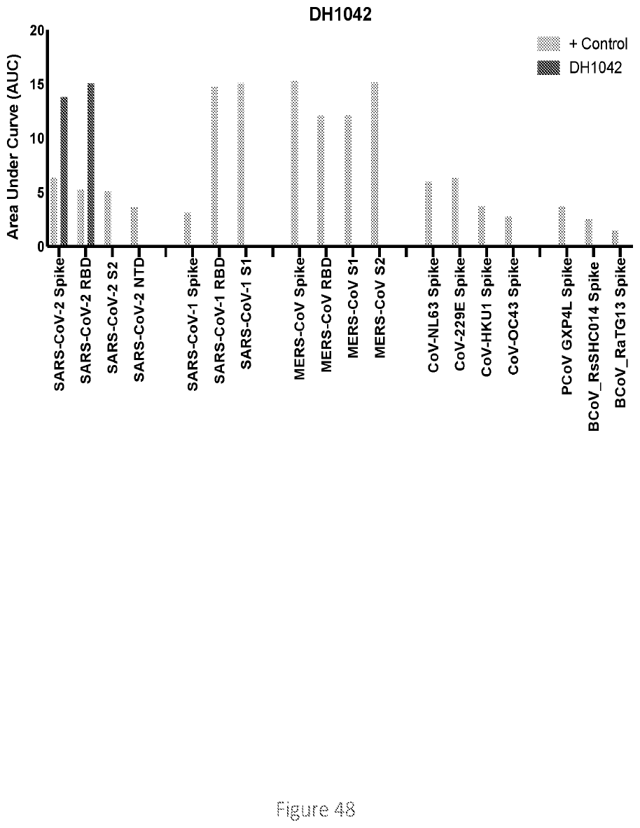

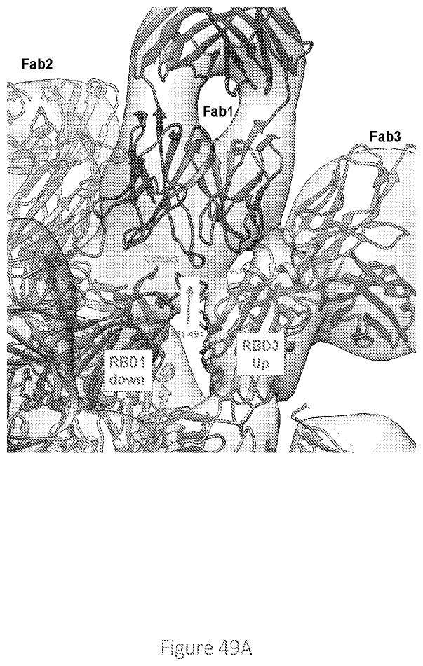

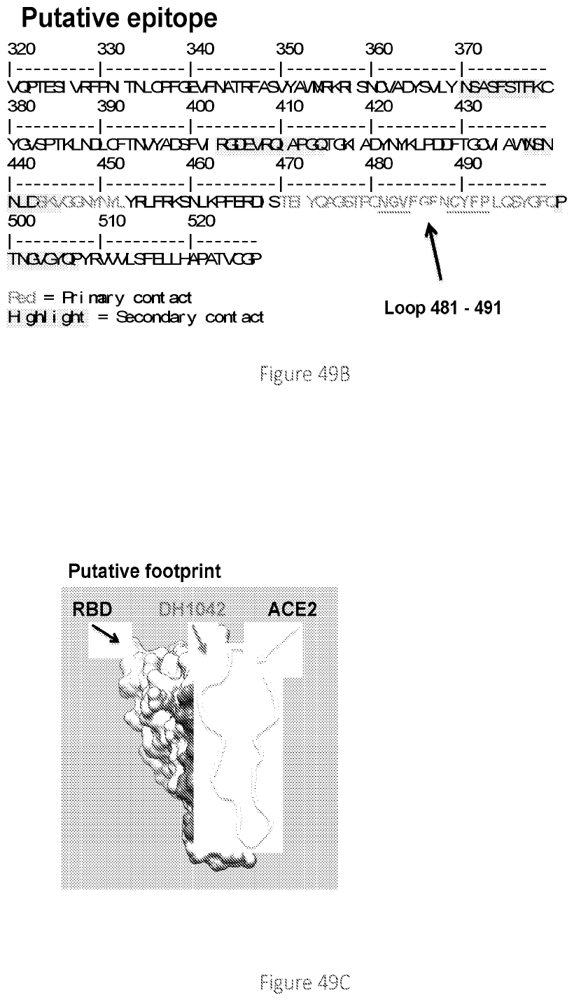

Negative stain electron microscopy was used to determine the footprint of DH1042 Fab binding to human SARS-Cov-2 spike (FIG. 49). DH1042 has a nearly vertical approach to spike binding and overlaps with the ACE2 (RBD binding) footprint. CryoEM studies are in progress to more finely map the DH1042-spike complex structure, epitope, and angle of approach of the antibody.

### In Vitro Blocking/Neutralization Potency

DH1042 blocks SARS-COV-2 Spike-RBD binding to human ACE2 and potently neutralizes both the D614 and G614 forms of the SARS-COV-2 virus. The latter is the predominant circulating strain in the United States (Table 15, FIG. 50). Specifically, DH1042 blocks SARS-COV-2 spike/RBD binding to human ACE2 in an ELISA-based assay with <200 ng/mL IC50 (Genscript), has a neutralization IC50 of 11 ng/ml in the VRC7480.D614G pseudovirus (293T/ACE2) assay, is a potent neutralizer (IC50 of 73 or 156 ng/mL) of authentic USA-WA-1-2020 (D614) virus in two independently run Plaque Reduction Neutralization Tests (PRNT) (3).

TABLE 15
In vitro potency against SARS-CoV-2

| | | ACE2 Blocking | Pseudovirus | Pseudovirus | Pseudovirus |
|---|---|---|---|---|---|
| Ab ID | Specificity | IC50 (μg/ml) | IC50 (μg/ml) | IC80 (μg/ml) | MPI |
| DH1042 | SARS-CoV-2- | <0.2 | 0.011 | 0.053 | 100 |
| | RBD | | | | |
| | | Duke RBL | Duke RBL | Bioqual | Bioqual |
| | | PRNT IC50 | PRNT IC90 | PRNT IC50 | PRNT IC90 |
| Ab ID | Specificity | (μg/mL) | (μg/mL) | (μg/mL) | (μg/mL) |
| DH1042 | SARS-CoV-2- | 0.073 | 0.625 | 0.156 | 0.625 |
| | RBD | | | | |

### SARS-COV-2 Spike/RBD Binding Kinetics

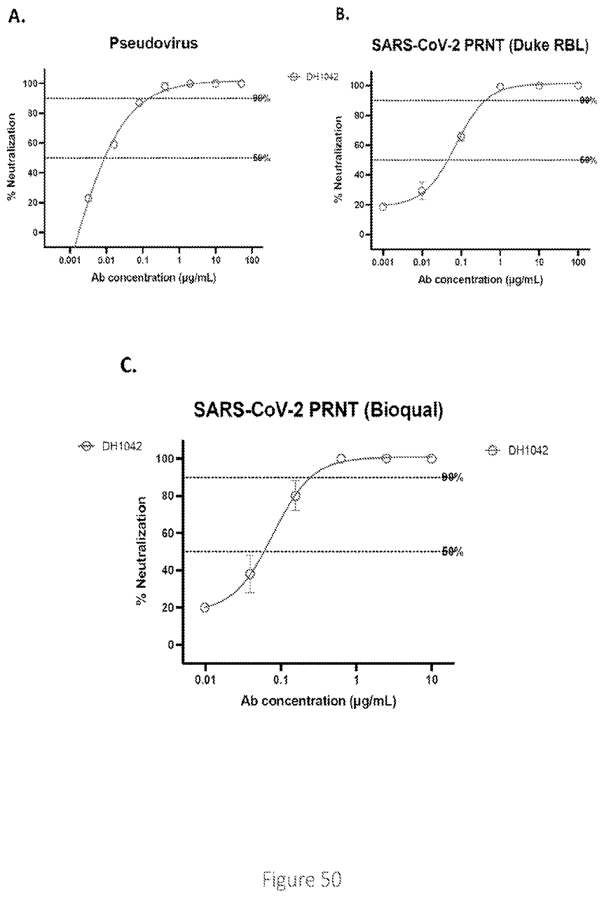

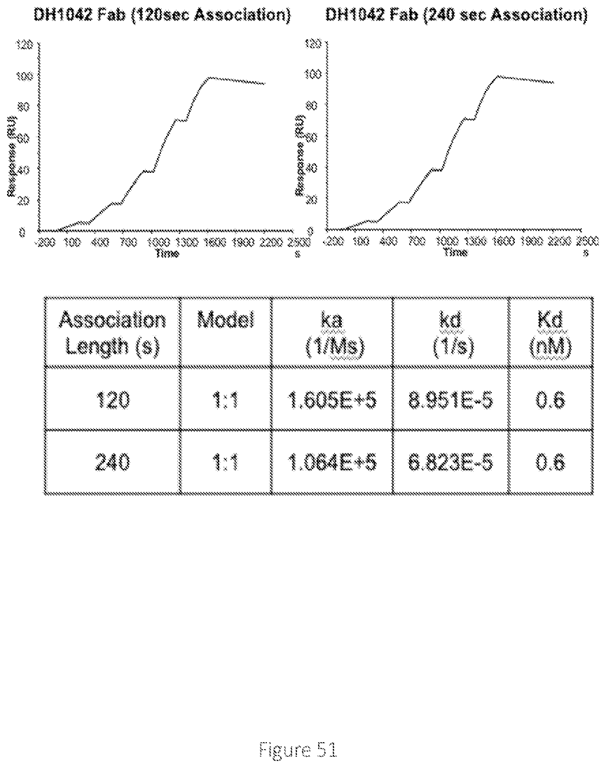

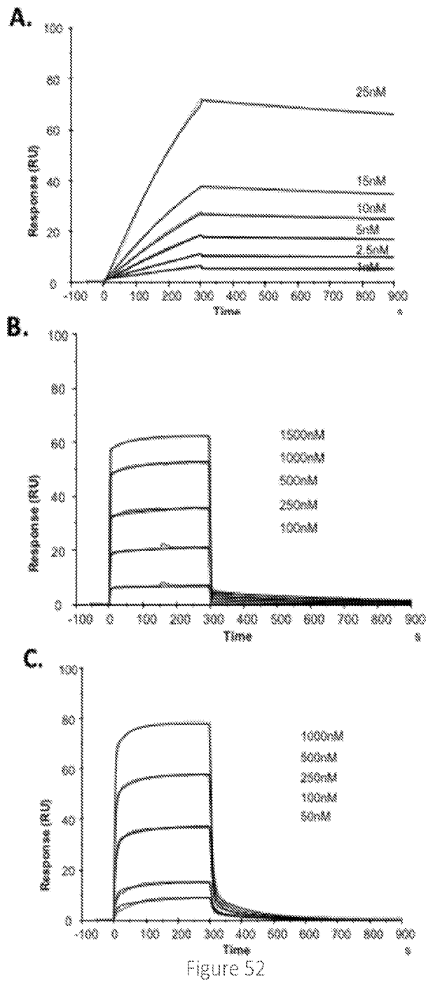

Using surface plasmon resonance (SPR, Bia3000) a kinetic study was performed to determine the binding kinetics of a Fab from DH1042 to SARS-COV-2 Spike. DH1042 has a Kd of 0.6 nM (FIG. 51). SPR was also used to determine binding kinetics of DH1042 (Hu IgG1-LS) to a panel of three human Fcg receptors (rhFcγRI/CD64, rhFcγRIIA/CD32, and rhFcγRIIIA/CD16a) (FIG. 52). Kd and on and off rates are summarized in Table 16.

TABLE 16
Kinetics of DH1042 (IgG1-LS) binding to
recombinant human Fc gamma receptors

| | ka | kd | Kd |
|---|---|---|---|
| Human Fc gamma Receptor | (1/Ms) | (1/s) | (nM) |
| FcgRI/CD64 | 4.140E+5 | 4.140E+5 | 4.140E+5 |
| FcgRIIA/CD32a | 5.646E+5 | 0.8099 | 1434 |
| FcgRIIIA/CD16a | 7.548E+5 | 0.3061 | 405.5 |

### Pre-clinical Safety (Recombinant DH1042 Protein)

Reactivity with Human Auto-antigens: DH1042 does not react with known human autoantigens. Using the Zeus Scientific ANA Hep-2 test we observed no significant staining with DH1042 (25 and 50 μg/mL tested) using standard immunofluorescent analysis (Zeus Scientific ANA Test: FIG. 53, Table 17). Cytoplasmic/nuclear staining is observed with the positive control antibody.

DH1042 was also screened for reactivity in the ZEUS AtheNA multiplex luminex bead-based assay to look for reactivity with nuclear and cytoplasmic proteins: SSA, Sjogren's syndrome antigen A: SSB, Sjogren's syndrome antigen B: Sm, small nuclear riboproteins: RNP, ribonucleoprotein: Scl 70, scleroderma 70; Jol, dsDNA, double-stranded DNA: Cent B, centromere B; and Histone. DH1042 was tested at multiple concentrations in the AtheNA assay along with negative and positive controls. DH1042 is negative for reactivity with all tested auto antigens.

TABLE 17
Quantitative AtheNA autoantigen panel

| | Test | | | | | | | | | |
|---|---|---|---|---|---|---|---|---|---|---|
| | Concentration | | | | | | | | | |
| mAb | (mg/mL) | SSA | SSB | Sm | RNP | Scl 70 | Jo 1 | dsDNA | Cent B | Histone |
| DH1042 | 50 | 2 | 4 | 3 | 6 | 1 | 2 | 0 | 56 | 9 |
| | 25 | 2 | 5 | 2 | 4 | 2 | 4 | 0 | 26 | 6 |
| | 12.5 | 3 | 2 | 1 | 0 | 0 | 1 | 2 | 12 | 3 |
| | 6.25 | 1 | 3 | 3 | 1 | 1 | 2 | 2 | 6 | 2 |
| Neg. | | — | — | — | — | — | — | — | — | — |
| Control | | | | | | | | | | |
| Pos. | | 447 | 485 | 839 | 1017 | 356 | 542 | 792 | 331 | 678 |
| Control | | | | | | | | | | |

* Negative is <100, positive is >120, and equivocal is 100 to 120.

### Tissue Cross-Reactivity Study (GLP):

A GLP Tissue Cross Reactivity study with DH1042 (aka Ab026103) on normal human tissues is in progress at Charles River Laboratories. An un-audited interim report was received Oct. 23, 2020 and has the following conclusion about DH1042 (aka Ab026103).

Staining with Ab026103 was limited to epithelial cells in the breast and/or pituitary. The epithelial staining with Ab026103 was cytoplasmic in nature, and monoclonal antibody binding to cytoplasmic sites in tissue cross-reactivity studies is considered of little to no toxicologic significance due to the limited ability of antibody drugs to access the cytoplasmic compartment in vivo (5, 6).

DH1042 mRNA Delivery

Messenger RNA (mRNA) is a promising new prophylactic delivery platform. While its application to prophylactic/therapeutic targets including infectious diseases is still in its infancy, work by our team and others has shown that it is transformative (7, 8). We have established a nucleoside-modified mRNA platform that has the combined benefits of potent delivery, safety and straightforward, rapid production suitable to deliver medical countermeasures. Specifically, target mRNA is in vitro transcribed with the modified nucleoside 1-methylpseudouridine (m1ψ) which we have demonstrated prevents innate immune sensing and increases mRNA translation in vivo (7, 8). Systemic administration of 1.4 mg/kg of nucleoside-modified mRNA encapsulated in lipid nanoparticles encoding the anti-HIV-1 antibody VRC01 resulted in plasma antibody titers of ˜170 mcg/mL 24 hours post-injection in humanized mice. Protective antibody titers were maintained for >1 week following a single administration and were maintained at ˜40 mcg/mL with repeat administration for over five weeks. Importantly, treatment with half this dose was sufficient to provide full protection from intravenous HIV-1 challenge, demonstrating that mRNA is a viable delivery platform for passive immunotherapy (7, 8).

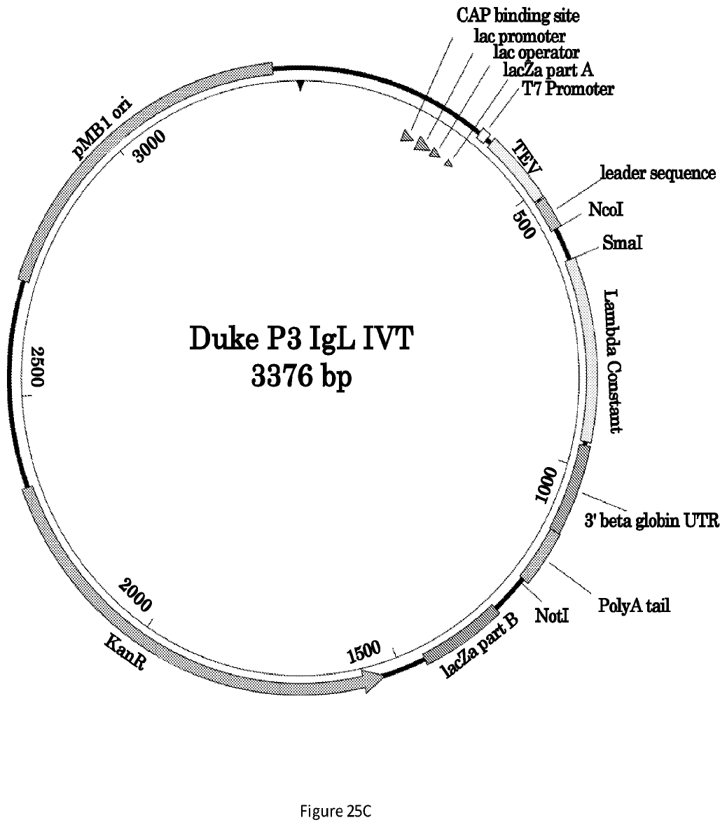

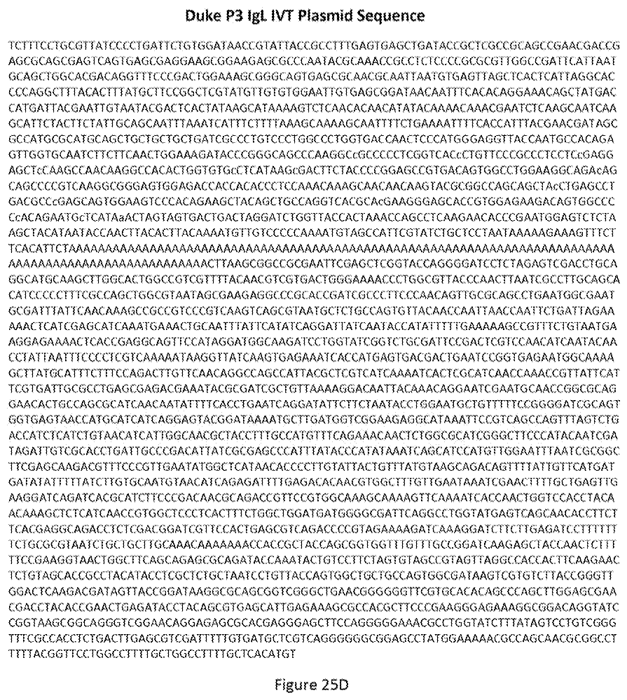

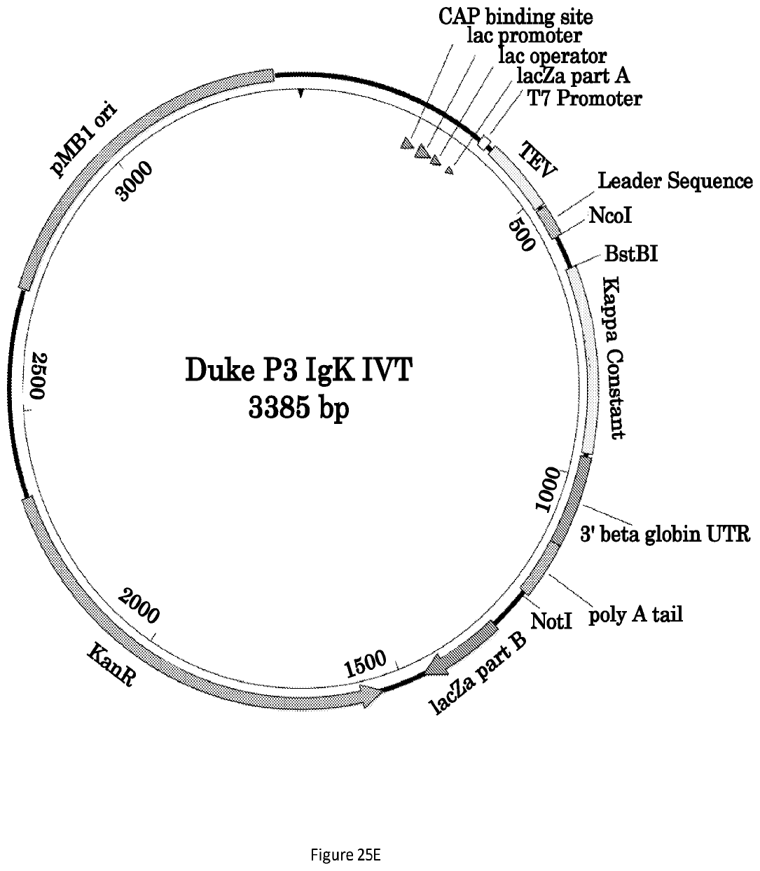

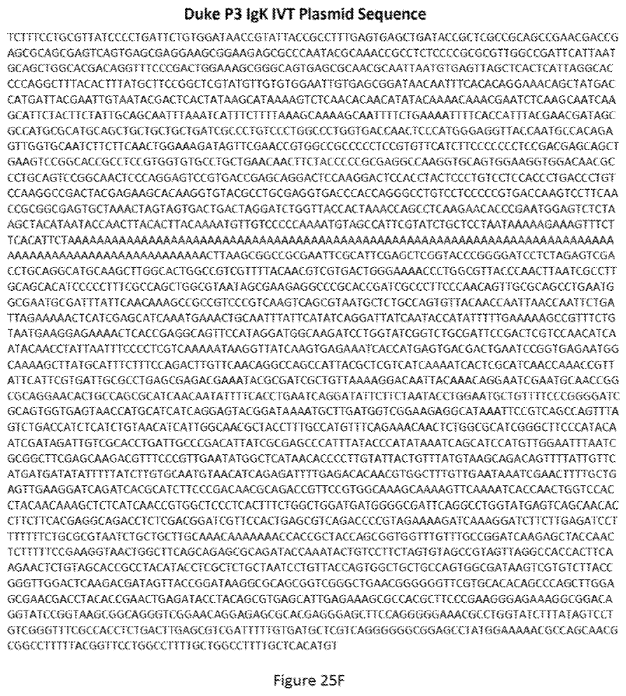

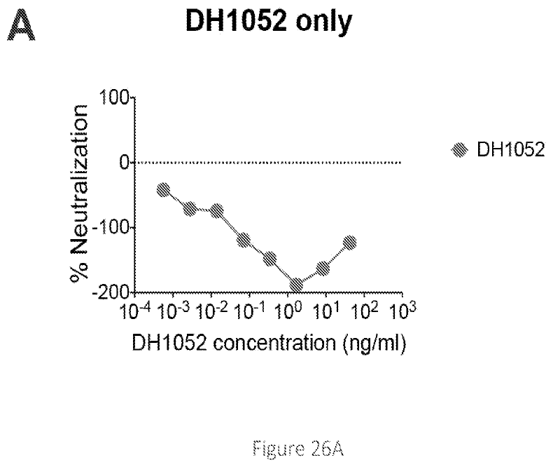

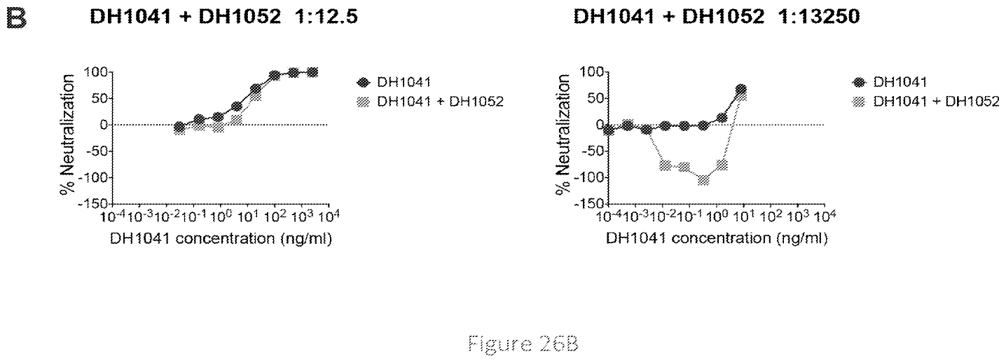

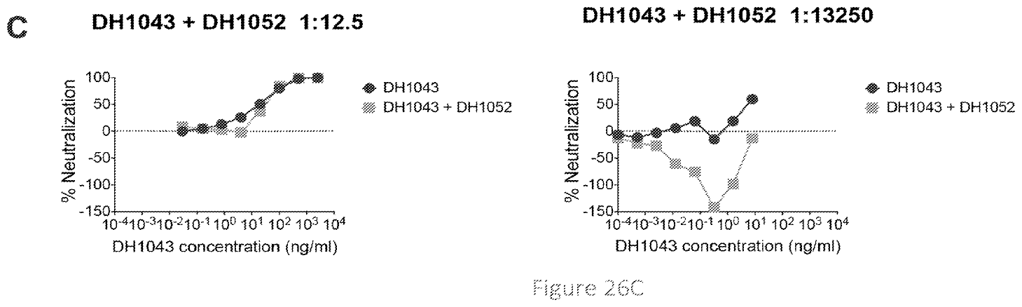

Synthesized DH1042 antibody variable heavy and light chain gene sequences were cloned into DNA vectors to serve as template for in vitro transcription. Three IVT plasmids have been generated with the optimal 5′ and 3′ untranslated regions, polyA tail and codon optimized human IgG1 LS, IgK or IgL constant region sequence. (FIG. 25). These plasmids are linearized using two unique restriction enzyme sites and the synthesized variable region DNA is cloned. Plasmids have kanamycin resistance for down-stream cGMP applications.

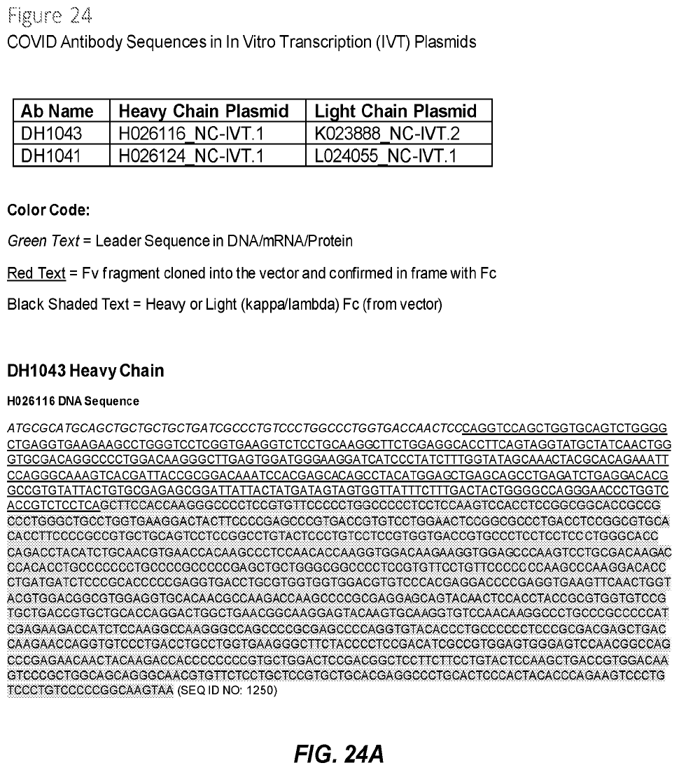

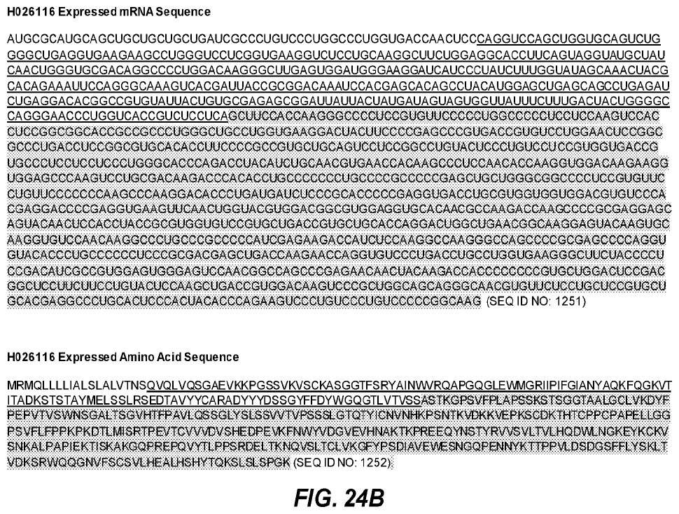

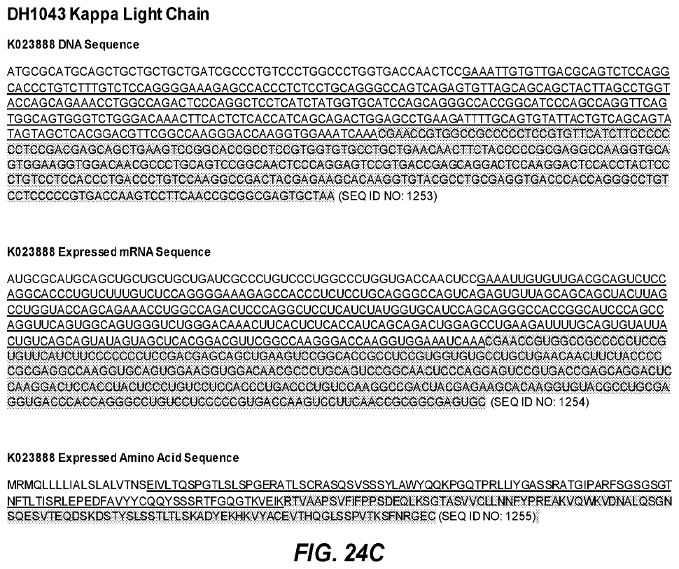

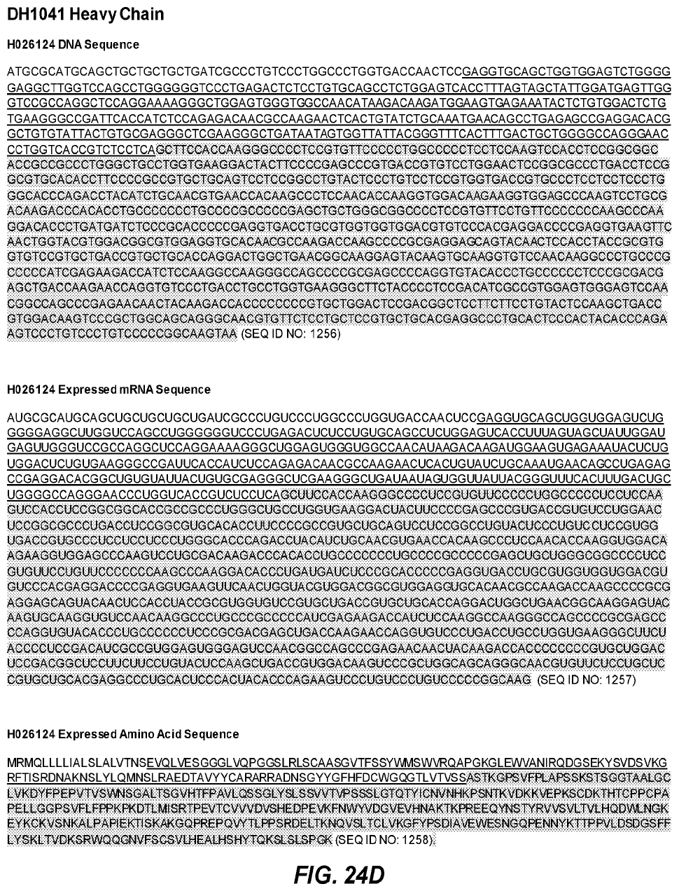

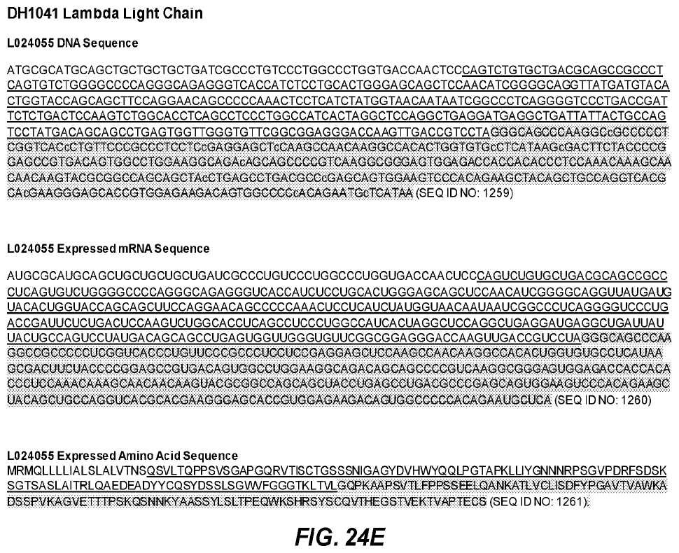

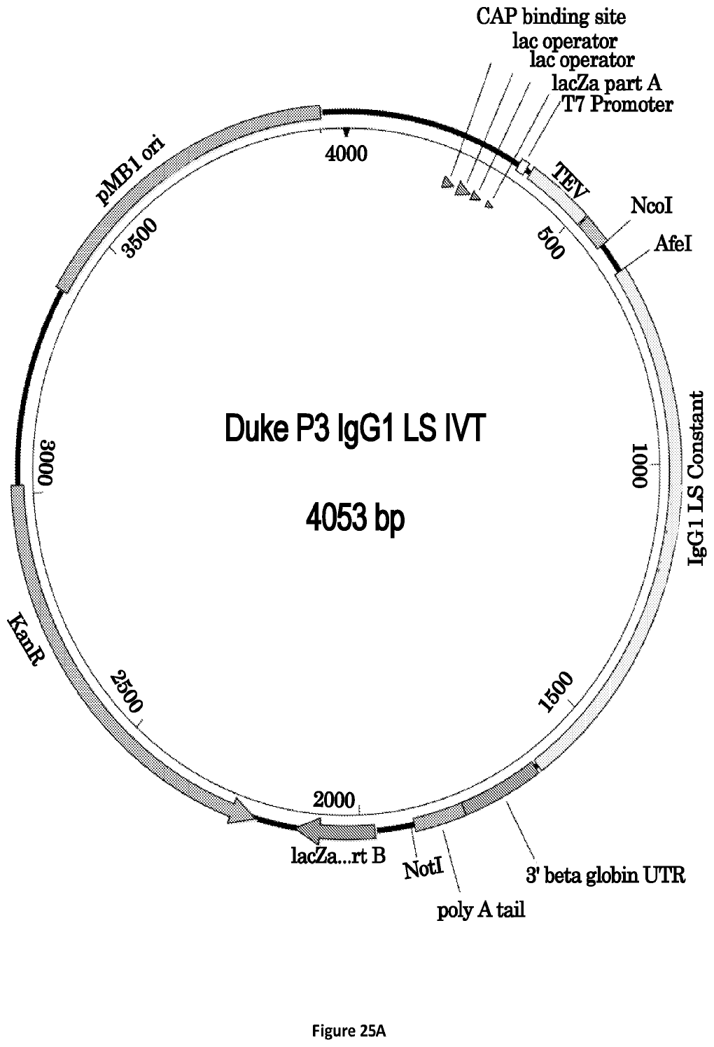

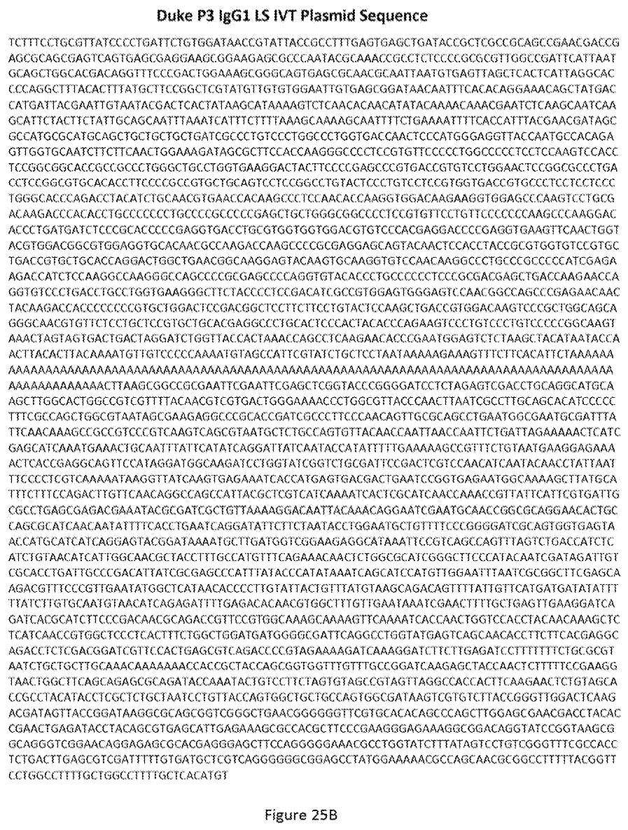

Only the IgH and IgK plasmids were needed for DH1042 as it contains a natural kappa light chain. In frame inserts were sequence confirmed and the two (H/K) IVT plasmids grown in large scale, purified with a low endotoxin plasmid prep kit and then used in IVT reactions to generate mRNA for in vitro and in vivo pre-clinical testing. It is critical that the endotoxin levels are below 2 EU/mg of plasmid DNA to prevent downstream complications with animal experiments. FIG. 24C summarizes the DNA, RNA and AA sequences for the heavy (H026103 NC-IVT.1) and light (K023879 NC-IVT.2) chain IVT plasmids. Green Text=Leader Sequence in DNA/mRNA/Protein, Red Text=Fv fragment cloned into the vector and confirmed in frame with Fc, and Black Text=human Heavy (lgG1-LS) or Light (kappa/lambda) Fc (from vector).

### IVT Plasmids Used to Make Modified RNA for Pre-clinical Animal Studies

Insert sequence confirmed Heavy and Light chain expressing IVT plasmids were expanded in E, coli XL-10 cells to ˜1000 mg/L at 37° C., using standard procedures. Plasmids were purified with commercial low endotoxin plasmid prep kits. Plasmid DNA was also run through a MiraCLEAN endotoxin removal step to ensure low endotoxin contamination of the resulting DNA template for IVT (FIG. 54). It is critical that the endotoxin levels are below 2 EU/mg DNA to prevent downstream complications with animal experiments. Plasmid concentration was determined by Nanodrop (Table 18). Endotoxin level quantified by EndosafeR nexgen-MCS™ benchtop spectrophotometer that uses disposable cartridges containing limulus amebocyte lysate (LAL), chromogenic substrate and controls to quantitate the unknown concentration of endotoxin in samples. Diluted RNA is added to the cartridge and the instrument calculates the endotoxin concentration based on the change of absorbance and comparison to an archived standard curve. An endotoxin concentration is reported if the internal spike recovery, which assesses assay interference and the test suitability, and an evaluation of % CV between replicates, meet acceptance criteria. Endotoxin levels meet criteria to be <2 EU/mg for down-stream use in pre-clinical animal models per Duke Institutional Animal Care and Use Committee (Table 18).

TABLE 18
DH1042 IVT Plasmids

| | | Conc. | Endotoxin Value |
|---|---|---|---|
| Antibody Name | Plasmid | (mg/mL) | (EU/mg) |
| DH1042 | H026103_NC-IVT.1 | 1.11 | <0.0455 |
| | K023879_NC-IVT.2 | 1.44 | <0.357 |

Production of Modified mRNA for Pre-clinical Animal Studies

### In Vitro Transcription (IVT):

Heavy and Light chain expressing IVT plasmids for DH1042 were linearized by digestion with the restriction enzyme Notl for approximately 2 hours at 37° C. A portion of the linearized DNA is mixed with the following RNA transcription components: T7 transcription buffer, T7 polymerase, ribonucleotide triphosphates (ATP, CTP, GTP), a modified nucleoside N (1)-methylpseudouridine (m1ψ) and Cap analog. The reaction mixture is incubated at 37° C. for approximately 2 hours. DNase is then added to digest the linearized template DNA, for 10-15 minutes at 37° C., in order to facilitate mRNA purification. Next, 50 mM ethylenediaminetetraacetic acid (EDTA) is added to minimize mRNA precipitation and the reaction is incubated for an additional 10-15 minutes at 37° C.

For pre-clinical studies heavy and light chain mRNAs are purified by LiCl precipitation, pelleted, washed with ethanol, briefly dried, and then resuspend in nuclease-free H2O. Purified RNA retains and bulks are handed off to DHVI GMP analytical group for pre-clinical testing (concentration, endotoxin, dsRNA).

mRNA Concentration and Yield:

RNA concentration is determined using the Molecular Devices M5 SpectraMax plate reader and the SpectraDrop Micro-Volume Microplate, which permits absorbance measurements with sample volumes as low as 2 uL. RNA samples are diluted in water and added to the microplate wells. Absorbance values are measured at 260 nm and normalized to a 1.0 cm path length. The concentration of RNA is determined by multiplying the normalized absorbance value at 260 nm by the concentration factor, 40, which is based on an A260 reading of 1.0 being equivalent to ˜40 μg/mL single-stranded RNA, and the appropriate dilution factor. DH1042 heavy and light chain pre-clinical study supply run concentration and yield is shown in Table 19.

TABLE 19
Concentration and yield for pre-clinical supply runs of mRNA

| | | Batch* | RNA | Total |
|---|---|---|---|---|
| | Plasmid DNA | Volume | Conc. | RNA |
| Antibody | Template | (mL) | (mg/mL) | (mg) |
| DH1042 | H026103_NC-IVT.1 | 9.3 | 1.024 | 9.52 |
| | K023879_NC-IVT.2 | 9.3 | 1.050 | 10.14 |

*pre-clinical supply run tested 2 Sep. 2020

### Endotoxin Testing:

Endotoxin levels in RNA product are quantified the Endosafe R nexgen-PTS™ handheld spectrophotometer that uses disposable cartridges containing limulus amebocyte lysate (LAL), chromogenic substrate and controls to quantitate the unknown concentration of endotoxin in samples. Diluted RNA is added to the cartridge and the instrument calculates the endotoxin concentration based on the change of absorbance and comparison to an archived standard curve. An endotoxin concentration is reported if the internal spike recovery, which assesses assay interference and the test suitability, and an evaluation of % CV between replicates, meet acceptance criteria. DH1042 heavy and light chain supply run RNA meet the <2 EU/mg pass criteria for down-stream use in pre-clinical animal models per Duke Institutional Animal Care and Use Committee.

TABLE 20
Endotoxin testing for pre-clinical supply runs of mRNA

| | | RNA | Endotoxin | Endotoxin |
|---|---|---|---|---|
| | | Conc. | Value | Value |
| Antibody | Plasmid DNA | (mg/mL) | (EU/mL)* | (EU/mg)** |
| DH1042 | H026103_NC-IVT.1 | 1.024 | <0.500 | <0.488 |
| | K023879_NC-IVT.2 | 1.050 | <0.500 | <0.476 |

*pre-clinical supply run tested 2 Sep. 2020, 0.500 represents <0.500 EU/mL, or below limit of quantitation of endotoxin chip
**0.### represents <0.### EU/mg, which is calculated by dividing <0.500 (EU/mL) by the RNA concentration (mg/mL)

### Double Stranded (ds) RNA Testing:

Residual dsRNA in pre-clinical supply run batch RNA is determined using a modified version of a previously described assay (9). The slot blot assay involves the capture of nucleic acid on a nylon membrane followed by addition of an anti-dsRNA antibody, J2 IgG2a. A signal is generated by the subsequent additions of goat anti-mouse IgG labeled with horseradish peroxidase (HRP) and chemiluminescent substrate. A semi-quantitative evaluation of the dsRNA content in an RNA sample is made by comparing the signal intensities of varying concentrations of the standard, a dsRNA ladder comprised of dsRNA fragments from 21-500 base pairs, and the sample. Both heavy and light chain RNA supply runs for DH1042 fall below 12.5 ng/200 ng load (FIG. 55).

### In Vitro RNA Launched Antibody Expression:

In vitro RNA expression of antibody will be assessed in HEK 293T cells through transient transfection using Lipofectamine™ Messenger Max™, a commercially available cationic lipid. Briefly, heavy and light chain RNA will be mixed at equimolar ratios, will be complexed with the lipid mixture and added to adherent HEK 293T cells. Cells are incubated for up to 72 hours to allow for the expression and secretion of antibody. Supernatants will be harvested, clarified, and the concentration of antibody in the supernatant is determined by biolayer interferometry (BLI) and Luminex bead-based binding.

In the BLI assay, a concentration series of Human IgG1 antibody reference standard or test sample will be captured on an anti-human Fc sensor. An evaluation of the concentration of antibody in the supernatant sample will be determined by extrapolation from the reference standard curve, where the response of the reference standard concentrations has been fit to a 4-parameter logistic equation. In the Luminex bead-based assay recombinant SARS-COV-2 spike protein will be conjugated to a fluorescent Luminex bead. A concentration series of DH1042 antibody reference standard or test sample will be captured on the bead. An evaluation of the concentration of antibody in the supernatant sample will be determined by extrapolation from the reference standard curve, where the response of the reference standard concentrations has been fit to a 4-parameter logistic equation.

LNP Encapsulation of mRNA for Pre-Clinical Animal Studies

Heavy and light chain mRNA are pre-mixed at an equimolar ratio for LNP encapsulation using proprietary lipid formulations and processes. Pre-clinical mRNA-LNP formulations must have <2 EU/mg endotoxin for down-stream use in pre-clinical animal models per Duke Institutional Animal Care and Use Committee. DH1042 mRNA was formulated with LNP-1 (Aka LNP-A) for in vivo PK and potency studies (FIG. 56).

### Pre-Clinical Pharmacokinetics (PK) of DH1042

### Protein Delivered DH1042 Antibody:

DH1042 protein dose and route PK studies were performed in Tg32 mice (also called hFcRn Tg32 or FcRn−/−hFcRn line 32 Tg) (FIG. 57). These mice carry a knock-out mutation for the mouse Fcgrt (Fc receptor, IgG, alpha chain transporter) gene and a transgene expressing the human FCGRT gene under the control of its own native promoter (hTg32). These mice are useful in evaluating the pharmacokinetics and pharmacodynamics of human immunoglobulin G (IgG) and Fc-domain based therapeutics.

Serum DH1042 antibody levels were determined with a Spike-specific luminex bead based anti-HuIgG quantitative assay. Recombinant DH1042 was used to generate an antigen-specific HuIgG standard curve to estimate ug/mL in the test samples.

The in vivo expressed maximum concentration (Cmax), time to reach maximum concentration (Tmax) and the human antibody half-life in the Tg32 mouse model was calculated (Table 21). Across both doses and routes, the average time to reach maximum expression level was 1.2 days and the half-life of the human antibody in a Tg32 mouse is estimated to be 8.5 days. Antibody protein dose and route PK studies in non-human primates and Syrian golden hamsters are planned to start November 2020.

TABLE 21
Pre-clinical PK - Protein delivered DH1042 antibody in mice

| | | | | Ab Dose | T half | Cmax | Tmax |
|---|---|---|---|---|---|---|---|
| Antibody | Model | Formulation | Route | (mg/kg) | (Days) | (ug/mL) | (Days) |
| DH1042 | Tg32 | Protein | IV | 5 | 8.9 | 25 | 1.0 |
| | Mouse | | | 10 | 9.9 | 62 | 1.6 |
| | | | IM | 5 | 7.6 | 19 | 1.0 |
| | | | | 10 | 7.0 | 58 | 1.0 |

* Dose adjusted from ug/mouse to mg/kg based on average mouse weight per study

mRNA-LNP delivered DH1042 antibody:

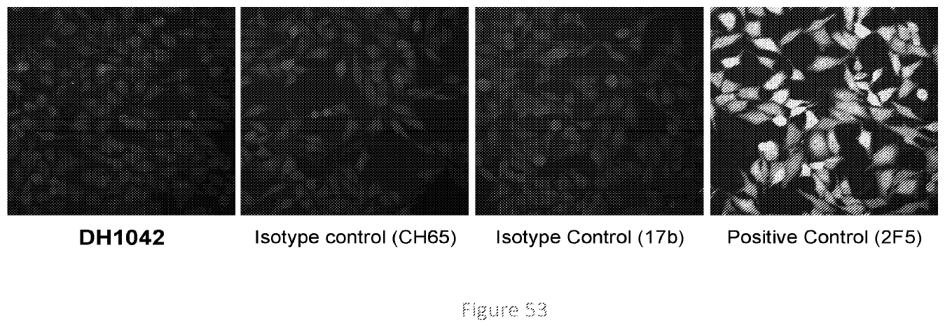

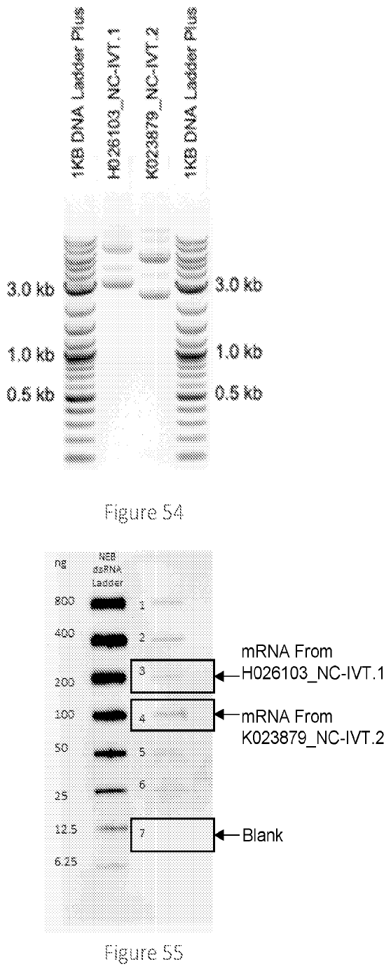

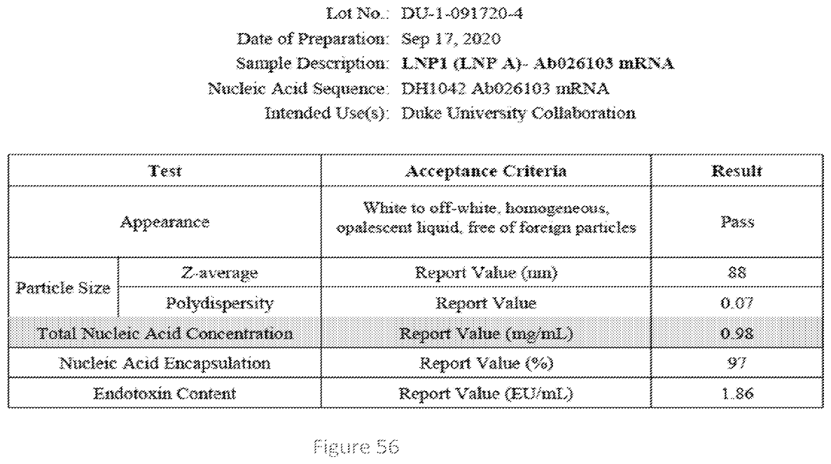

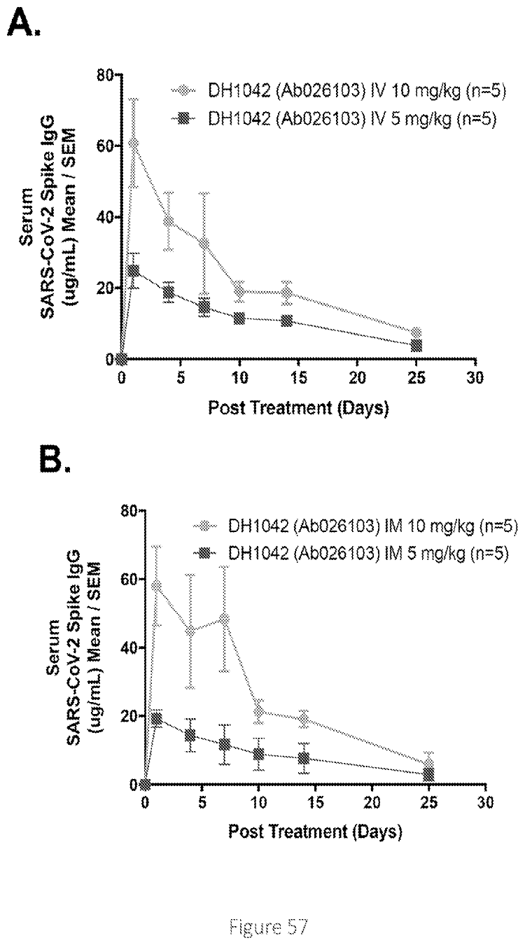

DH1042 mRNA-LNP dose and route PK studies were performed in Tg32 mice as described herein (FIG. 58), mRNA ug/mouse doses were selected for IV gene-delivered approach that approximated Cmax with recombinant protein in platform development studies. DH1042 antibody levels (expressed in vivo from mRNA) were determined with a Spike-specific luminex bead based anti-HuIgG quantitative assay as described herein. FIG. 59 shows PK studies in hamsters.

The in vivo expressed antibody maximum concentration (Cmax), time to reach maximum concentration (Tmax) and the human antibody half-life in the Tg32 mouse model was calculated (Table 22). Across doses, the average time to reach maximum expression level was 4.0 days and the half-life of the human antibody in a Tg32 mouse is 4.3 days.

TABLE 22
Pre-clinical PK - mRNA-LNP delivered DH1042 antibody in mice

| | | | | RNA Dose | T half | Cmax | Tmax |
|---|---|---|---|---|---|---|---|
| Antibody | Model | Formulation | Route | (mg/kg)* | (Days) | (ug/mL) | (Days) |
| DH1042 | Tg32 | mRNA-LNP-1 | IV | 0.5 | 3.4 | 26.7 | 4.0 |
| | Mouse | | | 0.9 | 5.6 | 63.2 | 3.4 |
| | | | | 1.9 | 3.9 | 182.6 | 4.6 |

*Dose adjusted from ug/mouse to mg/kg based on average mouse weight per study

One metric for the mRNA-delivered platform was to exceed 10 μg/mL within 72 hours and therefore we have accomplished this with all test doses of formulated DH1042 mRNA in Tg32 mice. It is important to note that no changes in animal body weight or temperature were observed in any of the pre-clinical mouse studies with IV administration of mRNA-LNP. DH1042 mRNA-LNP dose and route PK studies in Syrian golden hamsters are planned.

### Pre-clinical In Vivo Potency of DH1042 (In Progress)

Protein delivered DH1042 antibody, Protection from SARS-COV-2 challenge in mice:

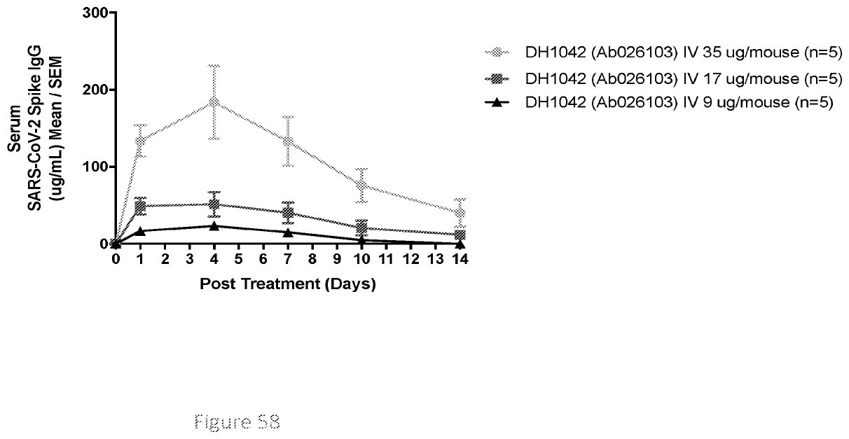

DH1042 recombinant protein treatment protects/reduces viral load in SARS-COV-2 pre- and post-exposure mouse challenge models. First, we tested DH1042 versus a control antibody (CH65, influenza neutralizing human IgG1 with LS backbone) in a Pre-exposure prophylaxis SARS-COV-2 acquisition mouse model (FIG. 60A). Aged (12 months old) BALB/c mice were injected intraperitoneally with 300 μg/mouse of recombinant DH1042 antibody, and challenged intranasally with a SARS-COV-2 mouse-adapted (MA) isolate (10) twelve hours later. Administration of DH1042 protected all mice from detectable infectious virus in the lungs 4 days after challenge (FIG. 60A).

Next we tested DH1042 versus control antibody in a therapeutic treatment model (FIG. 60B). Aged (12 months old) BALB/c mice injected intraperitoneally with 300 ug/mouse of recombinant DH1042 antibody twelve hours after SARS-COV-2 infection. Significantly reduced lung infectious virus titers, with 10/10 of the mice having undetectable infectious virus in the lung 4 days after viral infection (FIG. 60B).

Thus, DH1042 potently protected mice from SARS-COV-2 infection when administered prophylactically or therapeutically.

mRNA-LNP delivered DH1042 antibody. Protection from SARS-COV-2 challenge in Syrian Golden hamsters:

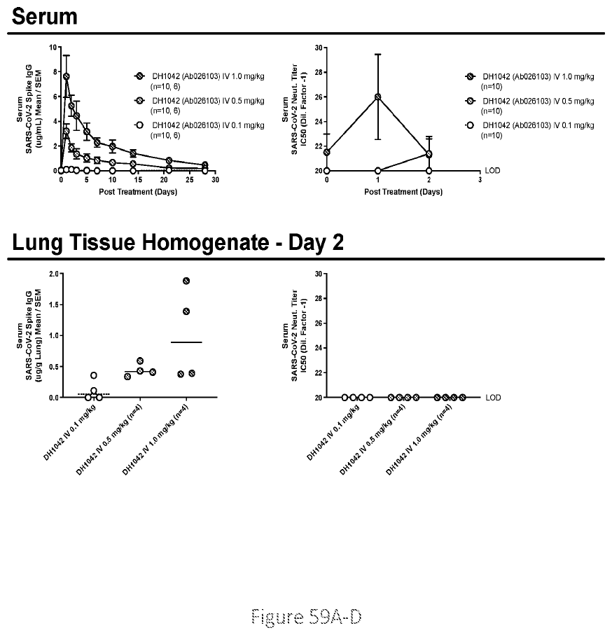

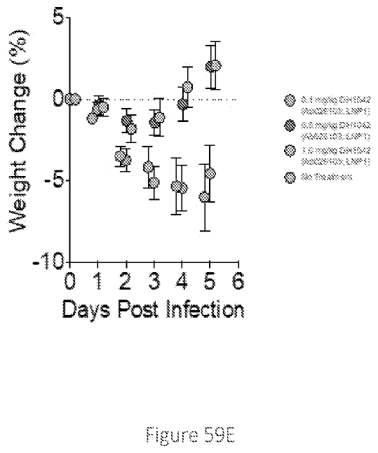

DH1042 mRNA-LNP prepared and used in the PK studies described herein was next used to determine in vivo potency of the DH1042 mRNA-launched antibody in protecting Syrian Golden hamsters from SARS-COV-2 viral challenge. We tested DH1042 mRNA versus challenge only in our pre-exposure prophylaxis SARS-COV-2 acquisition hamster model (FIG. 61). Hamsters were injected IV with 1 mg/kg formulated DH1042 RNA, and challenged intranasally with a SARS-COV-2 24 hours later. Administration of DH1042 protected all hamsters form virus-induced weight loss and there was undetectable infectious virus in oral swabs (qPCR) 5 and 7 days after challenge (FIG. 61).

Thus, DH1042 mRNA-LNP produced antibody in vivo and potently protects hamsters from SARS-COV-2 infection.

Protein Delivered DH1042 Antibody. Protection from SARS-COV-2 Challenge:

DH1042 pre-exposure protection studies in Syrian golden hamsters with wild-type SARS-COV-2 are in progress in the Duke Regional Biocontainment Laboratory ABSL3. Animals were treated IP with 5 or 10 mg/kg DH1042 recombinant Ab 24 hours prior to intranasal viral challenge and monitored for humane endpoints and viral load.

DH1042 pre-exposure protection studies in K18-hACE2 mice with wild-type SARS-CoV-2 are planned in the Duke Regional Biocontainment Laboratory ABSL3. Animals will be treated IP and IM with 1-10 mg/kg DH1042 recombinant Ab 24 hours prior to intranasal viral challenge and monitored for humane endpoints and viral load.

### Human Antibodies Against SARS-COV-2 (COVID)-19):

DH1042 is one of a panel of over 1,737 SARS-COV-2 binding/neutralizing antibodies isolated by the platform and approach described herein. Two additional potent neutralizing antibodies in this panel (DH1041 and DH1043) are very similar to DH1042 but have an unfavorable human tissue cross reactivity profile. Although these two antibodies are not progressing for human use, we do have substantial mouse and non-human primate (NHP) in vivo potency data that may inform our understanding of the potential for a closely related antibody (DH1042) to protect from viral challenge.

DH1041, DH1043 and DH1042 have nearly identical in vitro neutralizing potency, binding and affinity characteristics. All three antibodies bind the spike protein with similar vertical angles of approach relative to the vertical axis of the S trimer and the epitopes of antibodies on the RBD overlap with that of the ACE-2 receptor (11), consistent with the observation that the binding of these antibodies blocks ACE-2 binding. It is of note that this target epitope for all three antibodies (FIG. 49) is also shared with a recently described antibody P2B-2F6 (12) and 2 single-chain nanobodies (13, 14).

Protein Delivered DH1041 Antibody. Protection from SARS-COV-2 Challenge in Mice:

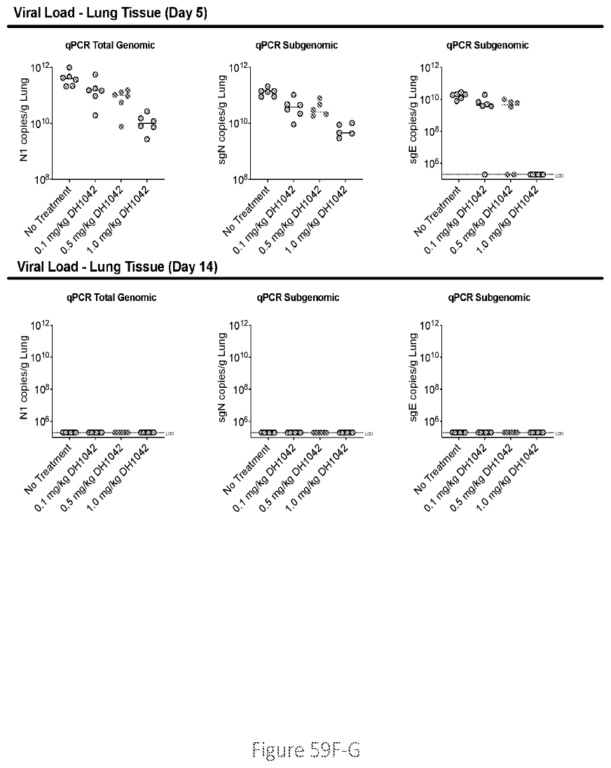

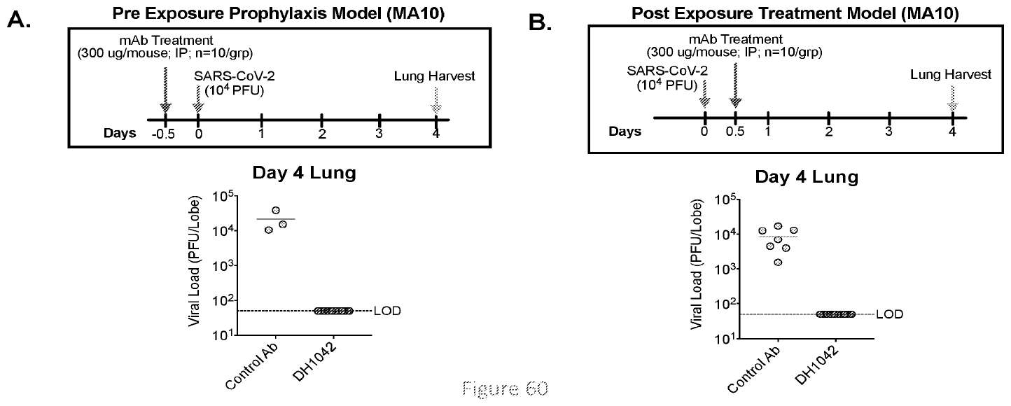

Although not advanced for down-selection, DH1041 and DH1043 were previously tested for in vivo function and demonstrate significant potency to protect/reduce viral load in SARS-COV-2 mouse and NHP challenge models. First we tested DH1041 versus a control antibody (CH65, influenza neutralizing human IgG1 with LS backbone) in a prophylaxis SARS-COV-2 acquisition mouse model (FIG. 62A). Aged (12 months old) BALB/c mice were injected intraperitoneally with 300 μg/mouse of recombinant DH1041 antibody, and challenged intranasally with a SARS-COV-2 mouse-adapted (MA) isolate (10) twelve hours later. Administration of DH1041 protected all mice from detectable infectious virus in the lungs 48 hours after challenge (FIG. 62A).

Next we tested DH1041 versus control antibody in a therapeutic treatment model (FIG. 62B). Aged (12 months old) BALB/c mice injected intraperitoneally with 300 ug/mouse of recombinant DH1041 antibody twelve hours after SARS-COV-2 infection. Significantly reduced lung infectious virus titers, with ⅗ of the mice having undetectable infectious virus in the lung 48 hours after viral infection (FIG. 19B).

Thus, DH1041 potently protected mice from SARS-COV-2 infection when administered prophylactically or therapeutically.

Protein Delivered DH1041 and DH1043 Antibody, Protection from SARS-COV-2 Challenge in Non-Human Primates:

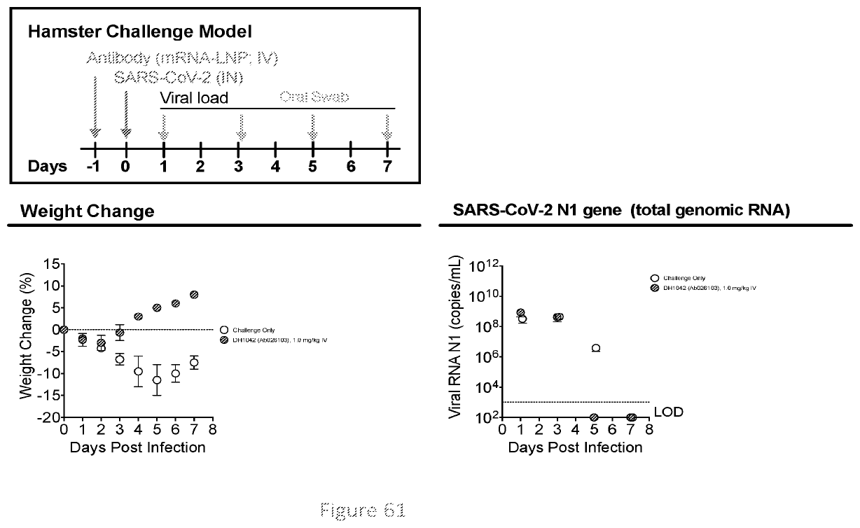

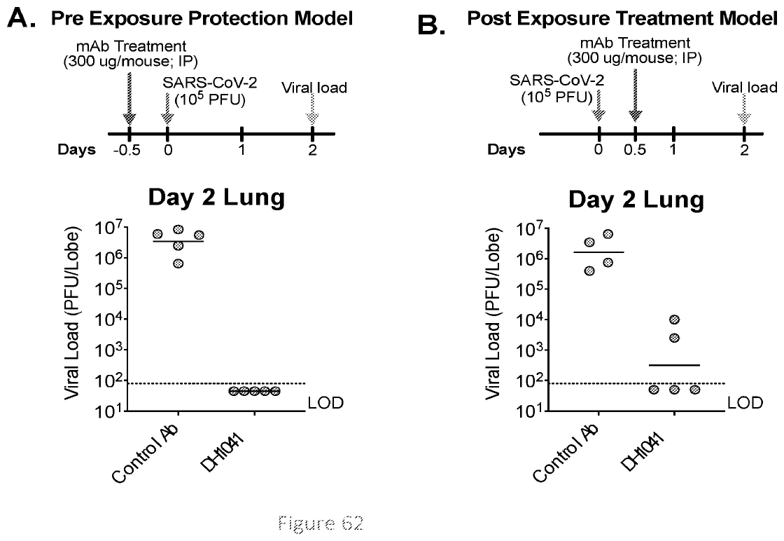

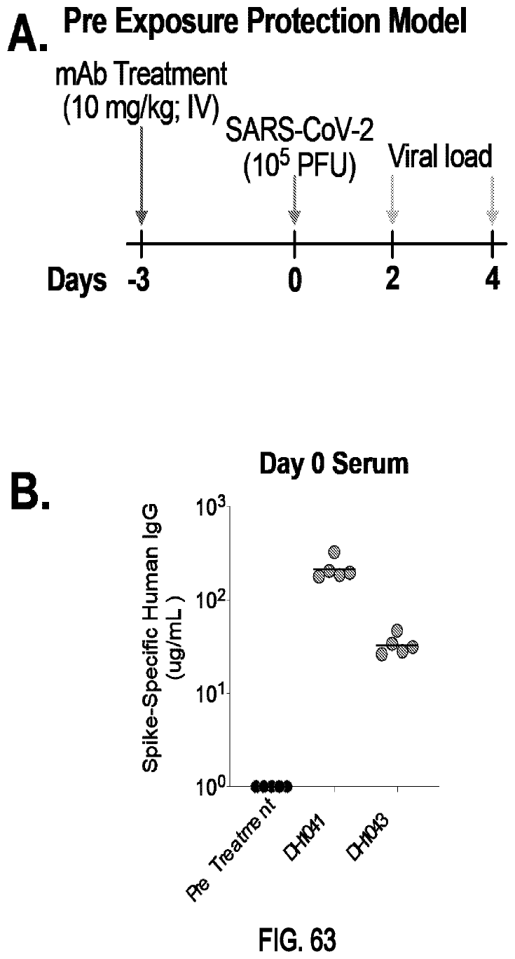

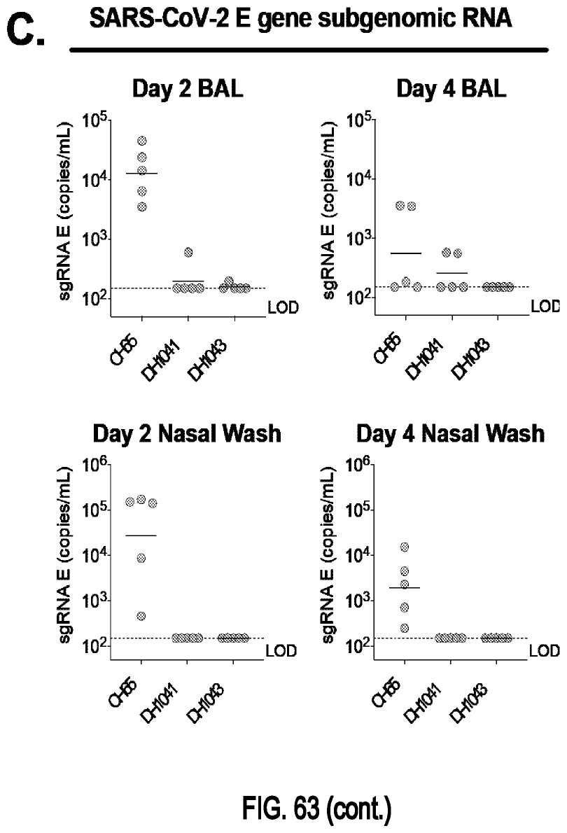

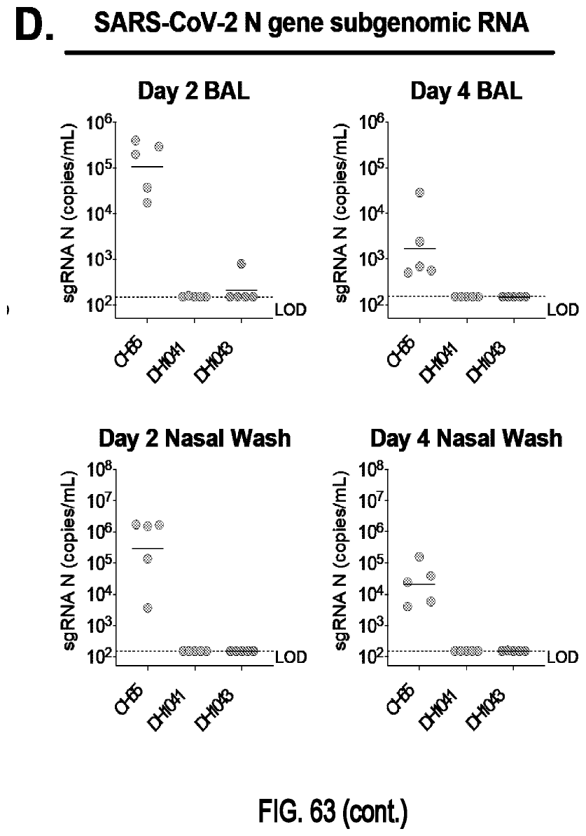

In an independent study run at Bioqual in NHPs, we assessed the in vivo potency of DH1041 and DH1043 in a cynomolgus macaque SARS-COV-2 intranasal/intratracheal challenge model (FIG. 63). Macaques were IV administered 10 mg/kg DH1041, DH1043, or CH65 control antibody three days prior to infection with SARS-COV-2. At the day of challenge, the animals had circulating levels of SARS-COV-2 specific DH1041 and DH1043 antibodies in the serum (FIG. 63B). Viral load was assessed on Day 2 and Day 4 post challenge and monitored by RT-CR for subgenomic RNA (sgRNA) for the E and N genes, indicative of viral load. E gene sgRNA and N gene sgRNA were significantly reduced in the lower and upper respiratory tract based on analyses of bronchoalveolar lavage fluid and nasal wash samples (FIGS. 63C and 63D). These results demonstrate that both DH1041 and DH1043, closely related antibodies to DH1042, potently protected NHPs from SARS-COV-2 infection when administered prophylactically.

### REFERENCES

1. Sempowski G D, Saunders K O, Acharya P, Wiehe K J, Haynes B F. Pandemic Preparedness: Developing Vaccines and Therapeutic Antibodies For COVID-19. Cell. 2020: 181 (7): 1458-63. Epub 2020 Jun. 4, doi: 10.1016/j.cell.2020.05.041. PubMed PMID: 32492407: PMCID: PMC7250787.
2. Liao H X, Levesque M C, Nagel A, Dixon A, Zhang R, Walter E, Parks R, Whitesides J, Marshall D J, Hwang K K, Yang Y, Chen X, Gao F, Munshaw S, Kepler T B, Denny T, Moody M A, Haynes B F. High-throughput isolation of immunoglobulin genes from single human B cells and expression as monoclonal antibodies. J Virol Methods. 2009: 158 (1-2): 171-9, doi: 10.1016/j.jviromet.2009.02.014. PubMed PMID: 19428587; PMCID: PMC2805188.
3. Berry J D, Jones S, Drebot M A, Andonov A, Sabara M, Yuan X Y, Weingartl H, Fernando L, Marszal P, Gren J, Nicolas B, Andonova M, Ranada F, Gubbins M J, Ball T B, Kitching P, Li Y, Kabani A, Plummer F. Development and characterisation of neutralising monoclonal antibody to the SARS-coronavirus. J Virol Methods. 2004: 120 (1): 87-96. Epub 2004 Jul. 6, doi: 10.1016/j.jviromet.2004.04.009. PubMed PMID: 15234813: PMCID: PMC7119589.
4. Ko SY, Pegu A, Rudicell R S, Yang Z Y, Joyce M G, Chen X, Wang K, Bao S, Kraemer T D, Rath T, Zeng M, Schmidt S D, Todd J P, Penzak S R, Saunders K O, Nason M C, Haase A T, Rao S S, Blumberg R S, Mascola J R, Nabel G J. Enhanced neonatal Fc receptor function improves protection against primate SHIV infection. Nature. 2014: 514 (7524): 642-5. Epub 2014 Aug. 15, doi: 10.1038/nature13612. PubMed PMID: 25119033: PMCID: PMC4433741.
5. Hall W, Price-Schiavi S, Wicks J, Rojko J. Tissue Cross-Reactivity Studies for Monoclonal Antibodies: Predictive Value and Use for Selection of Relevant Animal Species for Toxicity Testing. In: J A C, editor. Preclinical Safety Evaluation of Biopharmaceuticals: A Science-Based Approach to Facilitating Clinical Trials: Wiley-Interscience: 2008, p. 208-40.
6. Leach M W, Halpern W G, Johnson C W, Rojko J L, MacLachlan T K, Chan C M, Galbreath E J, Ndifor A M, Blanset D L, Polack E, Cavagnaro J A. Use of tissue cross-reactivity studies in the development of antibody-based biopharmaceuticals: history, experience, methodology, and future directions. Toxicologic pathology. 2010: 38 (7): 1138-66. Epub 2010 Oct. 12, doi: 10.1177/0192623310382559. PubMed PMID: 20926828.
7. Pardi N, Secreto A J, Shan X, Debonera F, Glover J, Yi Y, Muramatsu H, Ni H, Mui B L, Tam Y K, Shaheen F, Collman R G, Kariko K, Danet-Desnoyers G A, Madden T D, Hope M J, Weissman D. Administration of nucleoside-modified mRNA encoding broadly neutralizing antibody protects humanized mice from HIV-1 challenge. Nat Commun. 2017:8:14630, doi: 10.1038/ncomms14630. PubMed PMID: 28251988: PMCID: PMC5337964.
8. Pardi N, Hogan M J, Pelc R S, Muramatsu H, Andersen H, DeMaso C R, Dowd K A, Sutherland L L, Scearce R M, Parks R, Wagner W, Granados A, Greenhouse J, Walker M, Willis E, Yu J S, McGee C E, Sempowski G D, Mui B L, Tam Y K, Huang Y J, Vanlandingham D, Holmes V M, Balachandran H, Sahu S, Lifton M, Higgs S, Hensley S E, Madden T D, Hope M J, Kariko K, Santra S, Graham B S, Lewis M G, Pierson T C, Haynes B F, Weissman D. Zika virus protection by a single low-dose nucleoside-modified mRNA vaccination. Nature. 2017: 543 (7644): 248-51, doi: 10.1038/nature21428. PubMed PMID: 28151488: PMCID: PMC5344708.
9. Kariko K, Muramatsu H, Ludwig J, Weissman D. Generating the optimal mRNA for therapy: HPLC purification eliminates immune activation and improves translation of nucleoside-modified, protein-encoding mRNA. Nucleic Acids Res. 2011: 39 (21): e142. Epub 2011 Sep. 6, doi: 10.1093/nar/gkr695. PubMed PMID: 21890902: PMCID: PMC3241667.
10. Dinnon K H, 3rd, Leist S R, Schafer A, Edwards C E, Martinez D R, Montgomery S A, West A, Yount B L, Jr., Hou Y J, Adams L E, Gully K L, Brown A J, Huang E, Bryant M D, Choong I C, Glenn J S, Gralinski L E, Sheahan T P, Baric R S. A mouse-adapted model of SARS-COV-2 to test COVID-19 countermeasures. Nature. 2020. Epub 2020 Aug. 28, doi: 10.1038/s41586-020-2708-8. PubMed PMID: 32854108.
11. Wang Q, Zhang Y, Wu L, Niu S, Song C, Zhang Z, Lu G, Qiao C, Hu Y, Yuen K Y, Wang Q, Zhou H, Yan J, Qi J. Structural and Functional Basis of SARS-COV-2 Entry by Using Human ACE2. Cell. 2020: 181 (4): 894-904 e9. Epub 2020 Apr. 11, doi: 10.1016/j.cell.2020.03.045. PubMed PMID: 32275855: PMCID: PMC7144619.
12. Ju B, Zhang Q, Ge J W, Wang R K, Sun J, Ge X Y, Yu J Z, Shan S S, Zhou B, Song S, Tang X, Yu J F, Lan J, Yuan J, Wang H Y, Zhao J N, Zhang S Y, Wang Y C, Shi X L, Liu L, Zhao J C, Wang X Q, Zhang Z, Zhang L Q. Human neutralizing antibodies elicited by SARS-COV-2 infection. Nature. 2020: 584 (7819): 115-+. PubMed PMID: WOS: 000554511300002.
13. Huo J D, Le Bas A, Ruza R R, Duyvesteyn H M E, Mikolajek H, Malinauskas T, Tan T K, Rijal P, Dumoux M, Ward P N, Ren J S, Zhou D M, Harrison P J, Weckener M, Clare D K, Vogirala V K, Radecke J, Moynie L, Zhao Y G, Gilbert-Jaramillo J, Knight M L, Tree J A, Buttigieg K R, Coombes N, Elmore M J, Carroll M W, Carrique L, Shah P N M, James W, Townsend A R, Stuart D I, Owens R J, Naismith J H. Neutralizing nanobodies bind SARS-COV-2 spike RBD and block interaction with ACE2. Nat Struct Mol Biol. 2020. PubMed PMID: WOS: 000548197600003.
14. Zhou D M, Duyvesteyn H M E, Chen C P, Huang C G, Chen T H, Shih S R, Lin Y C, Cheng C Y, Cheng S H, Huang Y C, Lin T Y, Ma C, Huo J D, Carrique L, Malinauskas T, Ruza R R, Shah P N M, Tan T K, Rijal P, Donat R F, Godwin K, Buttigieg K R, Tree J A, Radecke J, Paterson N G, Supasa P, Mongkolsapaya J, Screaton G R, Carroll M W, Gilbert-Jaramillo J, Knight M L, James W, Owens R J, Naismith J H, Townsend A R, Fry E E, Zhao Y G, Ren J S, Stuart D I, Huang K Y A. Structural basis for the neutralization of SARS-COV-2 by an antibody from a convalescent patient. Nat Struct Mol Biol. 2020. PubMed PMID: WOS: 000559353900001.

### Example 7 Effect of Natural Mutations of SARS-COV-2 on Spike Structure, Conformation and Antigenicity

The data in this example demonstrate further the breadth of the antibody DH1047 against different variant spikes, including different versions of the South African spike and the Brazilian P. I spike.

### Binding of SARS-COV-2 S Protein Variants to ACE2 Receptor and Antibodies

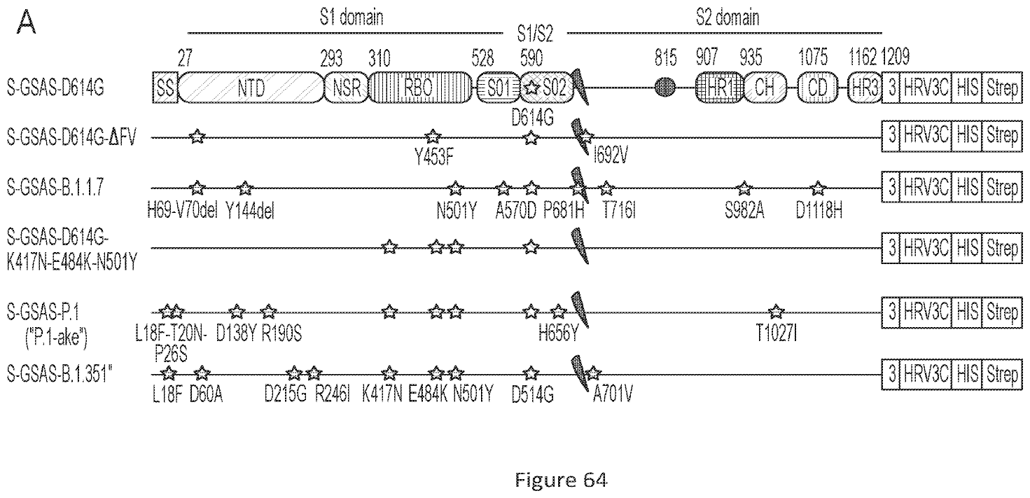

We used the previously described S-GSAS-D614G S ectodomain as template here (See FIG. 64) (referred to as “D614G spike” in the rest of the manuscript). This template includes SARS-COV-2 S residues 1-1208, a “RRAR” to “GSAS” substitution that renders the furin cleavage site inactive, a foldon trimerization motif at the spike C-terminus, followed by a C-terminal TwinStrep tag. All purified S proteins showed similar migration profiles on SDS-PAGE and size exclusion chromatography (SEC), with high-quality spike preparations confirmed by negative stain electron microscopy (NSEM).

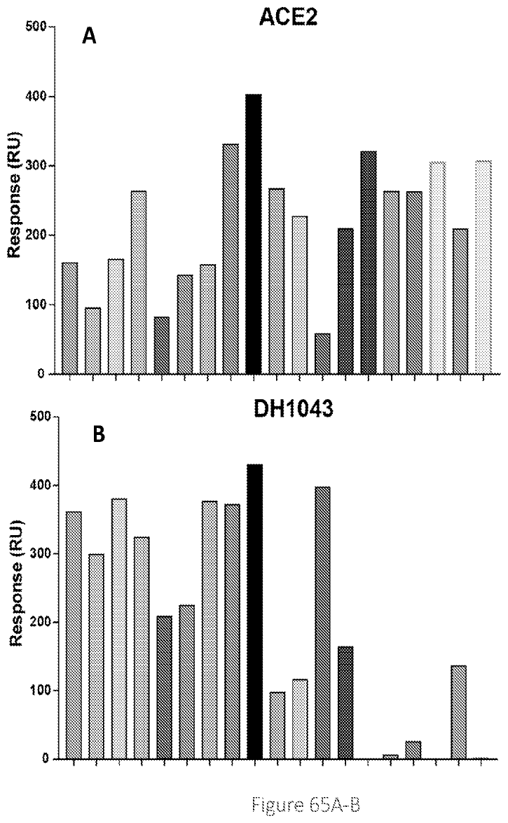

We measured spike binding to the ACE2 receptor ectodomain and to Abs using surface plasmon resonance (SPR) and ELISA (FIGS. 64, 65-66). Abs included RBD-directed, potent nAbs DH1041 and DH1043, whose epitopes overlap with the ACE2 binding site, RBD-directed highly cross-reactive nAb DH1047 that neutralizes SARS-COV-1, SARS-COV-2 and bat CoVs, NTD-directed nAbs DH1050.1 and DH1050.2 that bind an antigenic supersite, NTD-directed non-neutralizing Ab (nnAb) DH1052, fusion peptide-directed cross-reactive Ab DH1058, and S2 glycan cluster directed nnAb 2G12. All variants bound ACE2 at higher levels compared to the D614G spike (FIGS. 65-66), with S-GSAS-B.1.1.7 (“B.1.1.7 spike”) displaying the greatest increase. DH1047 showed similar binding levels to all spike variants (FIGS. 65-66), consistent with neutralization of B.1.1.7 and B.1.351 by DH1047. The RBD-directed nAb DH1041 showed similar binding levels to the B. 1.1.7 and D614G spikes, consistent with its neutralization of the B. 1.1.7 pseudovirus. The S-GSAS-D614G-K417-E484K-N501Y (or the “triple mutant spike”) showed reduced binding to RBD-directed nAbs DH1041 and DH1043. These results are consistent with the inability of Class 2 RBD binding Abs, where the E484K substitution occurs within the epitope, to neutralize variants that harbor the E484K substitution.

We tested several variants in the B.1.351 spike backbone (FIGS. 64, 65-66). We found that the commonly occurring 242-244 deletion, and a rare R246I substitution that is included in some reagent panels and candidate vaccine, can each impact not only binding of NTD-directed Abs, but also of RDB-directed Abs DH1041 and DH1043. While binding of NTD-directed nAbs DH1050.1 and DH1050.2 to B.1.1.7 and B.1.351 spikes was dramatically reduced. binding to triple mutant spike and S-GSAS-P.1 (or “P.1-like spike”) remained unchanged. This is consistent with neutralization data, where mAbs 5-24 and 4-8 that target the same antigenic supersite as DH1050.1, lost activity against B.1.351 but neutralized P.1.

The binding data were consistent with published neutralization data. B. 1.1.7 spike affinity for ACE2 was ˜5-fold improved over the D614G spike a result of the N501Y substitution. We measured nM affinity of the B. 1.1.7 spike for NTD-directed nAb DH1050.1. albeit at substantially reduced binding levels relative to the D614G spike (FIGS. 65-66), consistent with impairment of the NTD antigenic supersite in B.1.1.7, while retaining robust binding to most RBD-directed antibodies.

In summary, the binding data are consistent with biological data obtained in in vitro neutralization assays, thus establishing that our SARS-COV-2 S ectodomain constructs are an effective mimic of native spikes and supporting their use for studying structural changes due to amino acid substitutions in spike variants.

SPR methods. Antibody binding to SARS-COV-2 spike and RBD constructs was assessed using SPR on a Biacore T-200 (Cytiva. MA, formerly GE Healthcare) with HBS buffer supplemented with 3 mM EDTA and 0.05% surfactant P-20 (HBS-EP+. Cytiva. MA). All binding assays were performed at 25° C. Spike variants were captured on a Series S Strepavidin (SA) chip (Cytiva. MA) by flowing over 200 nM of the spike for 60 s at 10 uL/min flowrate. The Fabs were injected at concentrations ranging from 0.625 nM to 800 nM (2-fold serial dilution) using the single cycle kinetics mode with 5 concentration per cycle. For the single injection assay, the Fabs were injected at a concentration of 200 nM. A contact time of 60s, dissociation time of 120 seconds (3600s for DH1047 for the single cycle kinetics) at a flow rate of 50 μL/min was used. The surface was regenerated after each dissociation phase with 3 pulses of a 50 mM NaH+IM NaCl solution for 10 s at 100 pL/min. For the RBDs, the antibodies were captured on a CM5 chip (Cytiva. MA) coated with Human Anti-Fc (using Cytiva Human Antibody Capture Kit and protocol), by flowing over 100 nM antibody solution at a flowrate of 5 L/min for 120s. The RBDs were then injected at 100 nM for 120 s at a flowrate of 50 μL/min with a dissociation time of 30 s. The surface was regenerated by 3 consecutive pulse of 3M MgCl₂ for 10s at 100 μL/min. Sensorgram data were analyzed using the BiaEvaluation software (Cytiva, MA).

ELISA assays. Spike ectodomains tested for antibody- or ACE2-binding in ELISA assays as previously described (58). Assays were run in two formats i.e., antibodies/ACE2 coated, or spike coated. For the first format, the assay was performed on 384-well plates coated at 2 μg/ml overnight at 4° C., washed, blocked and followed by two-fold serially diluted spike protein starting at 25 μg/mL. Binding was detected with polyclonal anti-SARS-COV-2 spike rabbit serum (developed in our lab), followed by goat anti-rabbit-HRP (Abcam, Ab97080) and TMB substrate (Sera Care Life Sciences, MA). Absorbance was read at 450 nm. In the second format, serially diluted spike protein was bound in wells of a 384-well plates, which were previously coated with streptavidin (Thermo Fisher Scientific, MA) at 2 μg/mL and blocked. Proteins were incubated at room temperature for 1 hour, washed, then human mAbs were added at 10 μg/ml. Antibodies were incubated at room temperature for 1 hour, washed and binding detected with goat anti-human-HRP (Jackson ImmunoResearch Laboratories, PA) and TMB substrate.

### Example 8 a Broadly Cross-Reactive and Neutralizing Antibody Protects Against Sarbecovirus Challenge in Mice

SARS-COV in 2003 and SARS-COV-2 variants of concern (VOC) can cause deadly infections, underlining the importance of developing broadly effective countermeasures against sarbecoviruses, which can be key in the prevention and mitigation of current and future zoonotic events. Here, we demonstrate the neutralization of SARS-COV, bat CoVs WIV-1, RsSHC014, and SARS-COV-2 variants D614G, B.1.1.7, B.1.351, P.1, B. 1.429, B. 1.526, B.1.617.1, and B.1.617.2 by a receptor-binding domain (RBD) human antibody DH1047. Prophylactic and therapeutic treatment with DH1047 demonstrated efficacy against SARS-CoV, WIV-1, RsSHC014, and SARS-COV-2 B.1.351 infection in mice. Binding and structural analysis showed high affinity binding of DH1047 to an epitope that is highly conserved among sarbecoviruses. Thus, DH1047 is a broadly protective antibody that can prevent infection and mitigate outbreaks caused by SARS-related strains and SARS-COV-2 variants. Our results argue that the conserved RBD epitope bound by DH1047 is a rational target for a universal Sarbecovirus vaccine.

The emergence of severe acute respiratory syndrome coronavirus (SARS-COV) in 2003 led to more than 8.000 infections and 800 deaths (1. 2). In 2019. SARS-COV-2 emerged in Wuhan. China (3). The spread of SARS-COV-2, the virus that causes coronavirus disease of 2019 (COVID-19) was rapid. By March 2020, the World Health Organization (WHO) had declared the SARS-COV-2 outbreak a global pandemic. By August 2021, more than 200 million people had been infected globally, resulting in >4.5 million deaths with the documented emergence of several SARS-COV-2 variants which can partially evade host immunity. Therefore, there is a need to develop safe and effective broad-spectrum countermeasures that can prevent the rapid spread and attenuate the severe disease outcomes associated with current and future SARS-related virus emergence events.

Highly pathogenic CoV outbreaks in humans can be of bat origin (4), and there is great genetic diversity among bat SARS-related viruses (5). Zoonotic CoVs of bat origin, such as RsSHC014 and WIV-1, can utilize the human ACE2 receptor for cell entry and infect human airway cells (6. 7), underlining their potential for emergence in naïve human populations. Moreover, existing therapeutic monoclonal antibodies against SARS-COV, and SARS-COV-2 mRNA vaccines do not fully protect against zoonotic SARS-related virus infection in vivo (6-8). Given the pandemic potential of SARS-related viruses, the development of broadly effective countermeasures, such as universal vaccination strategies (8-10), and coronavirus (CoV) cross-reactive monoclonal antibodies is a global health priority. Moreover, given the emergence of the SARS-COV-2 variants that are partially or fully resistant to some neutralizing antibodies authorized for COVID-19 treatment (11-14), there is a need to develop mAb therapies that are broadly effective against the SARS-COV-2 variants and zoonotic SARS-related viruses that may emerge in the future.

The receptor binding domain (RBD) of SARS-COV-2 is one of the targets for highly potent neutralizing antibodies. Despite the high degree of genetic diversity within the RBD in SARS-related viruses (5), antibodies can be engineered to recognize diverse SARS-related viruses. Rappazzo et al, recently reported that an engineered RBD-directed antibody. ADG-2, neutralized SARS-related viruses and protected against SARS-COV and wild type SARS-COV-2 (15). Therefore, the RBD of sarbecoviruses contains conserved epitopes that are the target of broadly neutralizing antibodies. In agreement with the notion that the RBD contains a conserved epitope shared among SARS. SARS-related. SARS-COV-2 and the variants, we have identified a CoV cross-reactive RBD protective antibody: DH1047 (16). Here, we demonstrate, using both pseudoviruses and live virus assays, that DH1047 neutralizes SARS-CoV. SARS-related bat viruses RsSHC014 and WIV-1, and all tested SARS-COV-2 variants of concern. Structural analysis with SARS-COV spike shows that DH1047 targets a highly conserved RBD region among the sarbecoviruses. We demonstrate that DH1047 provides prophylactic and therapeutic protective activity against pathogenic SARS-COV, RsSHC014, WIV-1, and SARS-COV-2 B.1.351 variant in mice. Thus, DH1047 is a pan-group 2B CoV protective antibody that can be used to prevent or treat SARS-COV-2 infections including variants of concern and has the potential to prevent disease from a future outbreak of a pre-emergent, zoonotic SARS-related virus strains that jump into naïve animal and human populations.

### Results

### The Identification of Broadly Cross-Binding and Neutralizing Antibodies

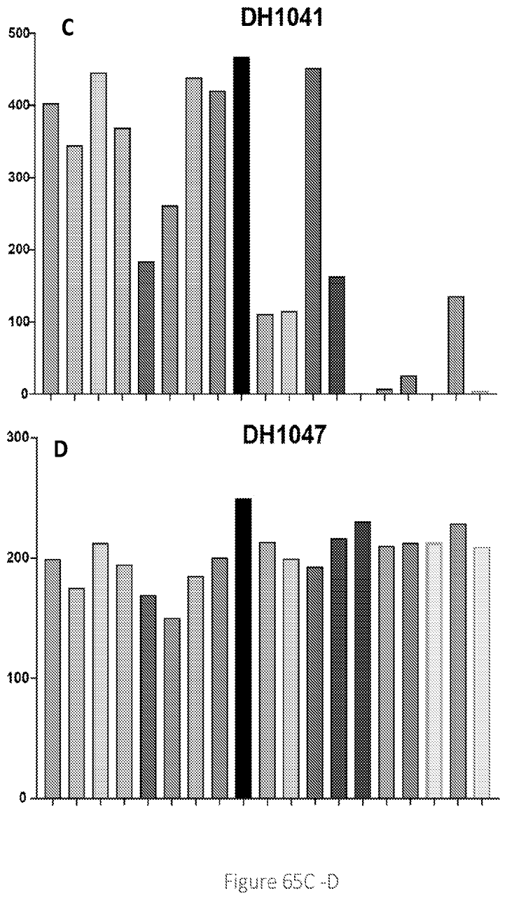

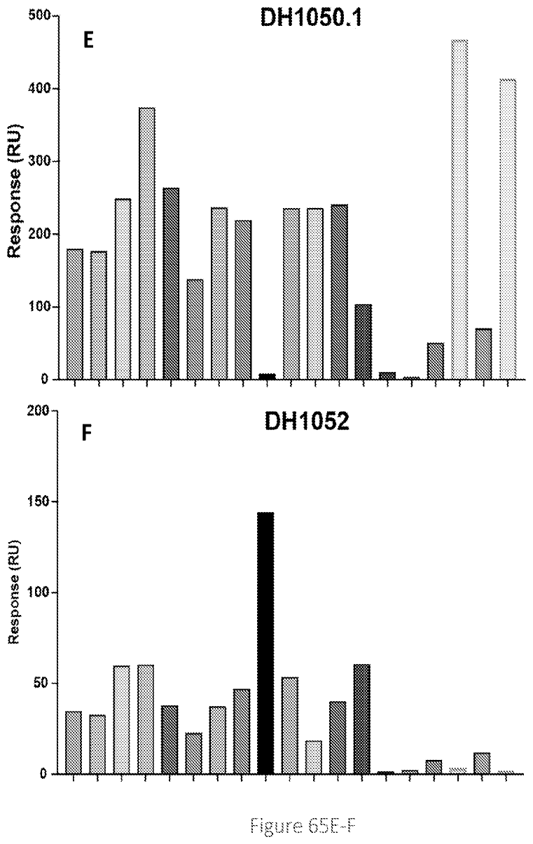

We previously isolated 1737 monoclonal antibodies (mAbs) from a SARS-COV convalescent patient 17 years following infection and a SARS-COV-2 convalescent patient from 36 days post infection (16). From this large panel of mAbs previously described by Li et al, we focused on 50 cross-reactive antibodies that bound to SARS-COV, SARS-COV-2, and other human and animal CoV antigens from genetically distinct sarbecoviruses. Importantly, sarbecoviruses vary in their ability to utilize human ACE2 and exhibit substantial genetic diversity in their RBDs (FIG. 67A) (16).

To examine if these 50 cross-neutralizing mAbs neutralized pandemic and zoonotic sarbecoviruses, we measured neutralizing activity against a SARS-COV-2 2AA Q498Y/P499T spike mutations in the mouse-adapted (MA) virus, SARS-COV, bat CoV WIV-1, and bat CoV RsSHC014 using live viruses, and found four broadly cross-reactive antibodies, DH1235, DH1073, DH1046, and DH1047 (FIG. 67).

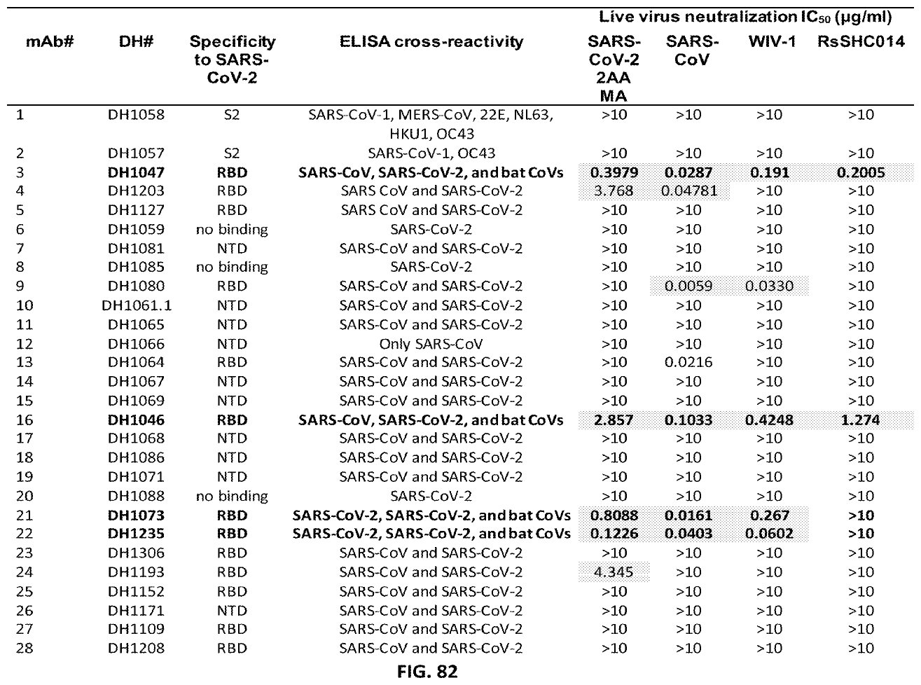

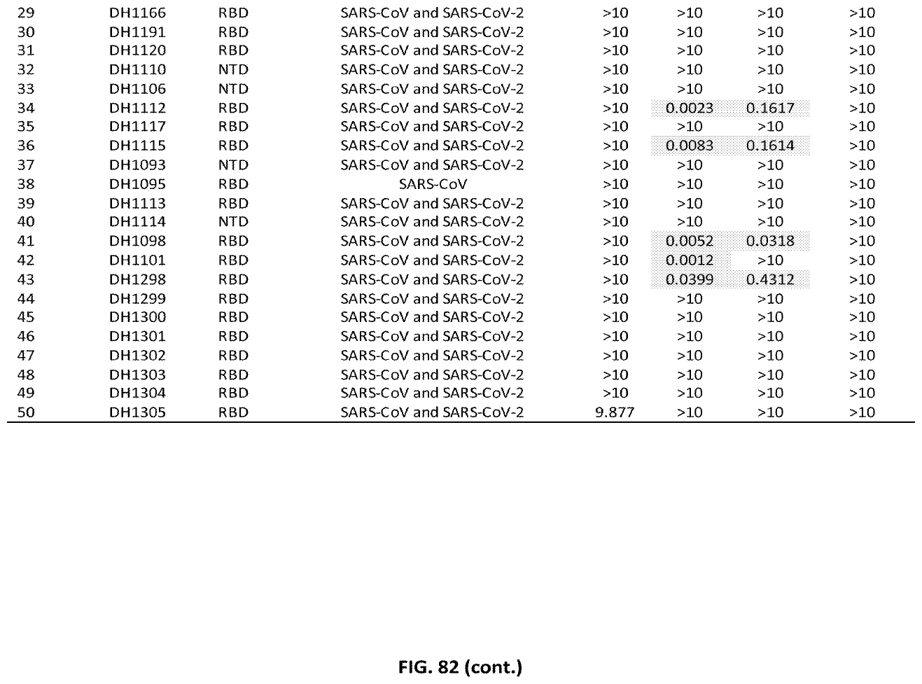

We focused our analyses on these four antibodies as they had broad neutralization. DH1235 neutralized SARS-COV-2 2AA MA, SARS-COV, and bat CoV WIV-1 with IC50 of 0.122, 0.0403, and 0.060 ug/ml, respectively (FIG. 67B and FIG. 82). DH1073 neutralized SARS-COV-2 2AA MA, SARS-COV, bat CoV WIV-1, and bat RsSHC014 with IC50 of 0.808, 0.016, 0.267, and 1.32 ug/ml, respectively (FIG. 67C and FIG. 82). DH1046 neutralized SARS-CoV-2 2AA MA, SARS-COV, bat CoV WIV-1, and bat CoV RsSHC014 with IC50 of 2.85, 0.103, 0.425, and 1.27 ug/ml, respectively (FIG. 67D and FIG. 82). DH1047 was the most potent, and neutralized SARS-COV-2 2AA MA, SARS-COV, bat CoV WIV-1, and bat CoV RsSHC014 with IC50 of 0.397, 0.028, 0.191, and 0.200 μg/ml, respectively (FIG. 67E and FIG. 82). As our study goal was to characterize the protective efficacy of cross-reactive antibodies, we compared the neutralization activity of DH1047 to two other mAbs currently in clinical trials: the cross-reactive ADG-2 (Adagio Therapeutics Inc-Adimab) and the potent REGN 10933 (Regeneron Pharmaceuticals) against SARS-COV-2 B.1.351. SARS-COV urbani, and RsSHC014 (FIG. 72) ADG-2 was engineered for increased potency whereas DH1047 was not (15). REGN 10933 had reduced neutralizing activity against SARS-COV-2 B.1.351 compared to both ADG-2 and DH1047 (FIG. 72). Like DH1047, ADG-2 effectively neutralized SARS-CoV urbani whereas REGN 10933 did not. Last, both ADG-2 and DH1047 neutralized RsSHC014 whereas REGN 10933 did not (FIG. 72).

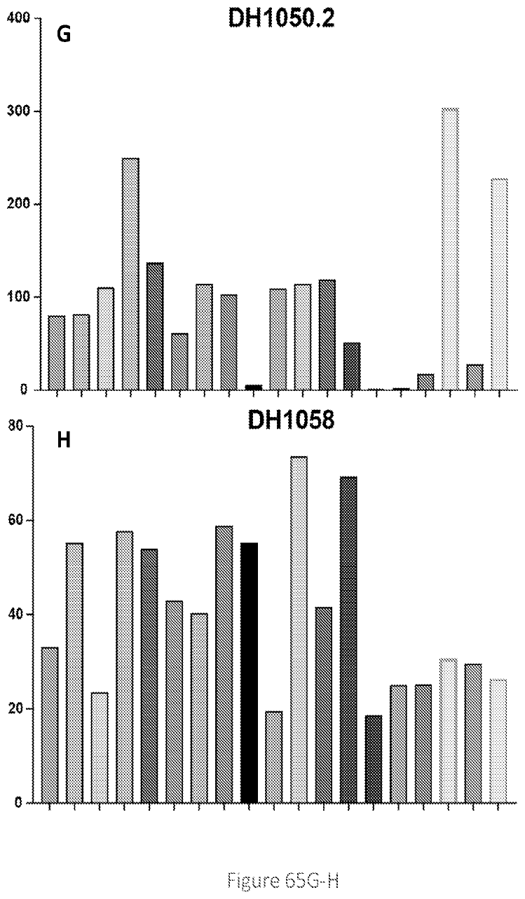

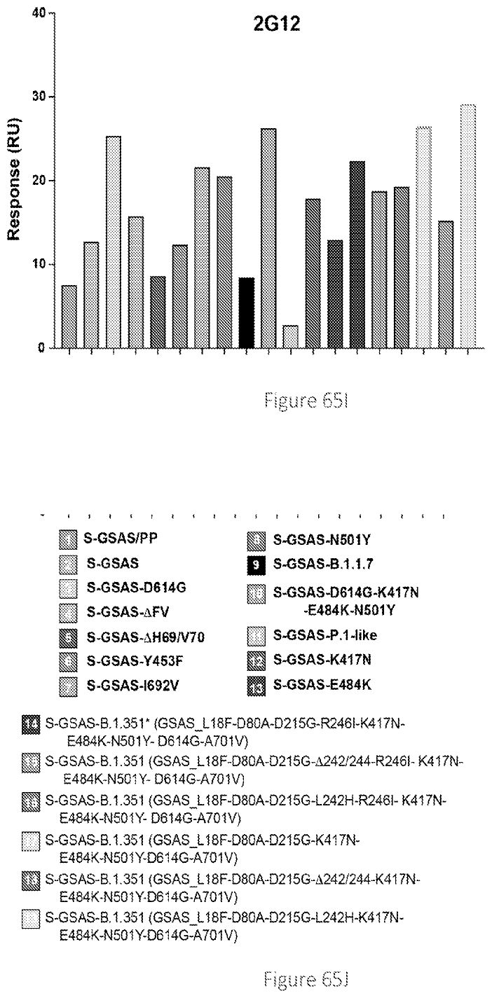

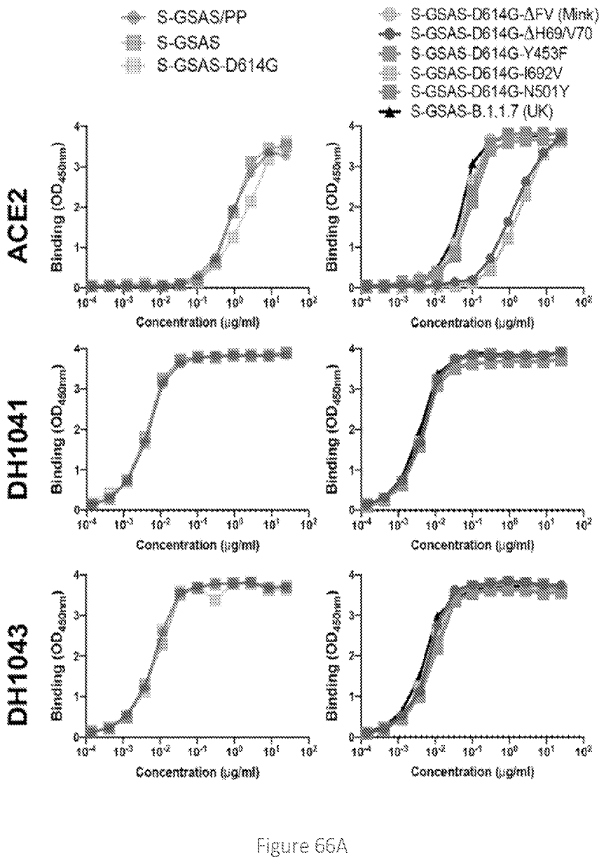

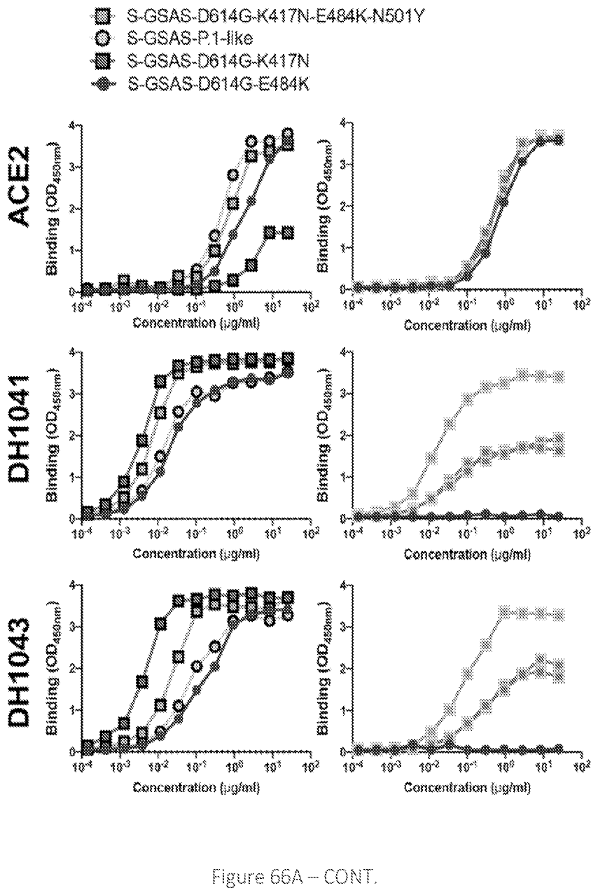

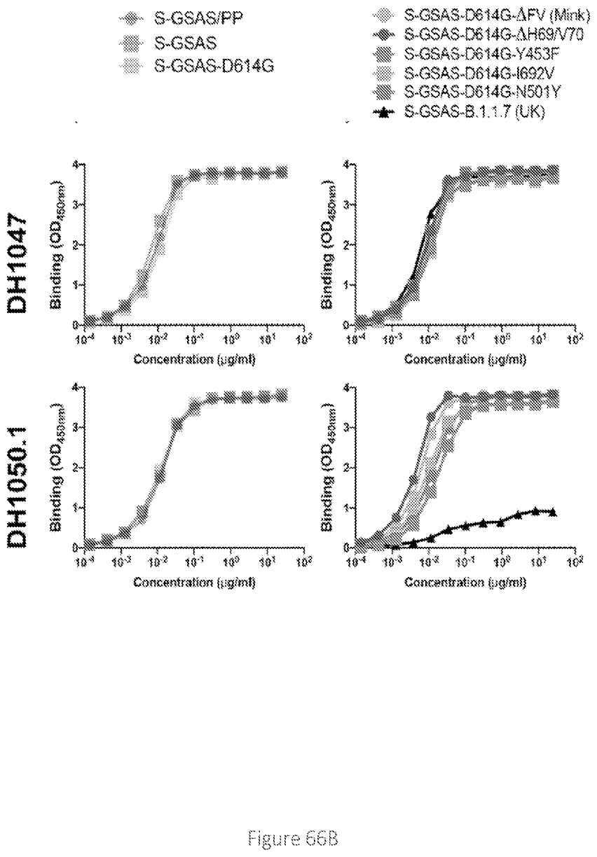

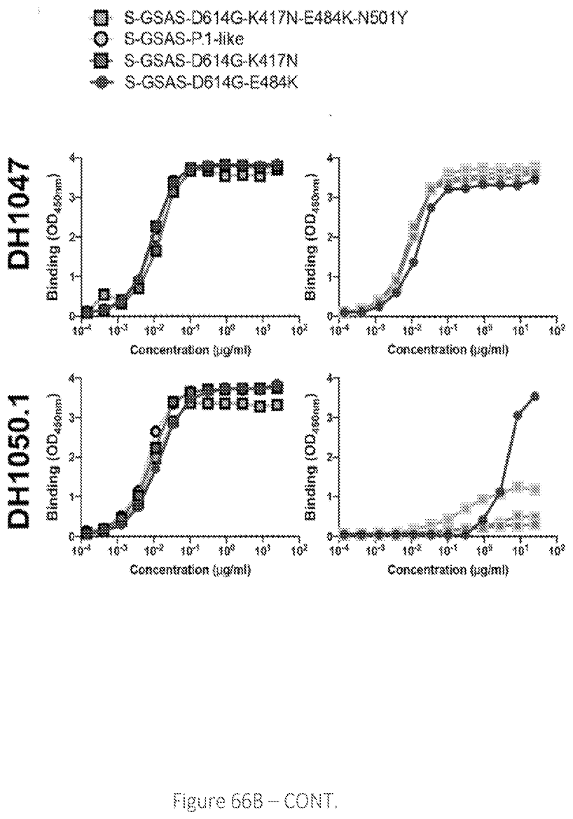

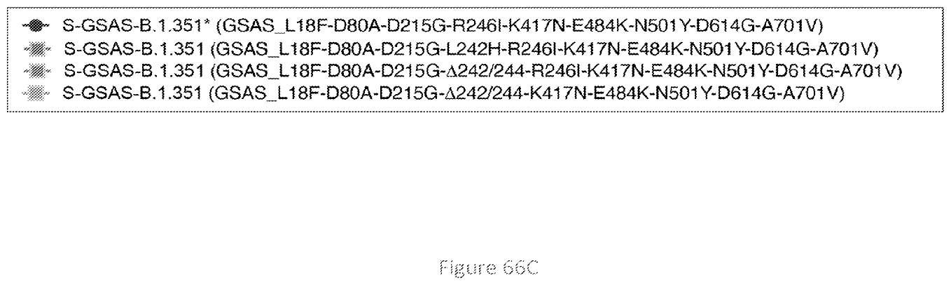

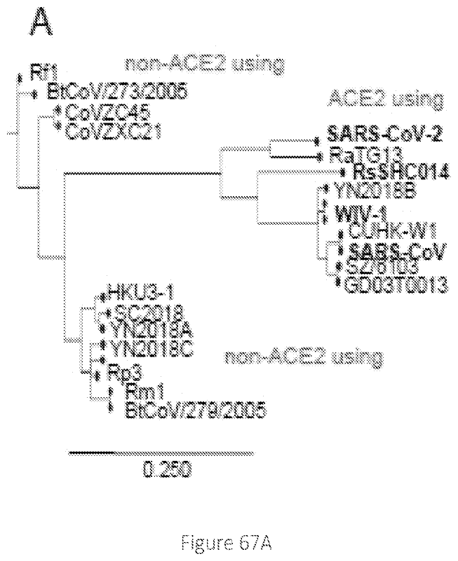

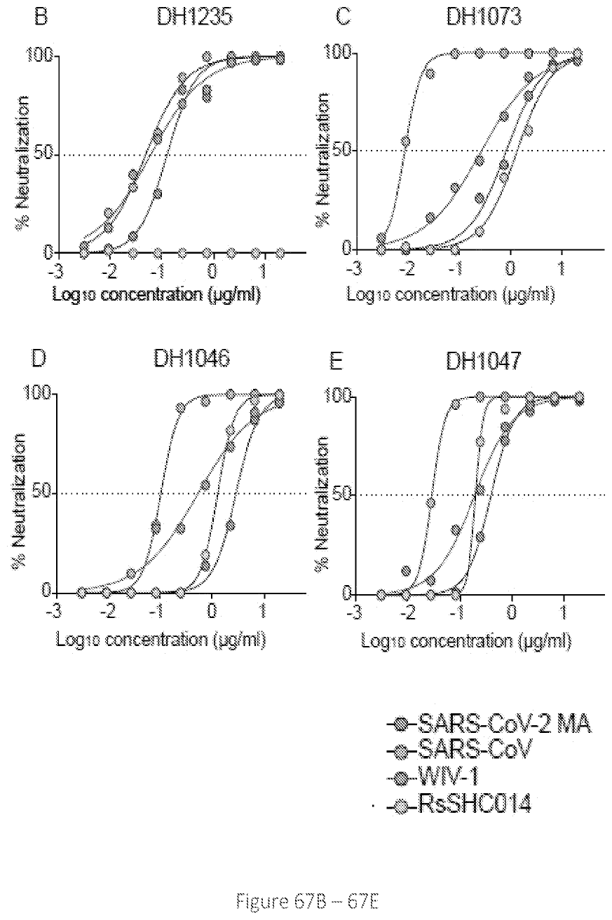

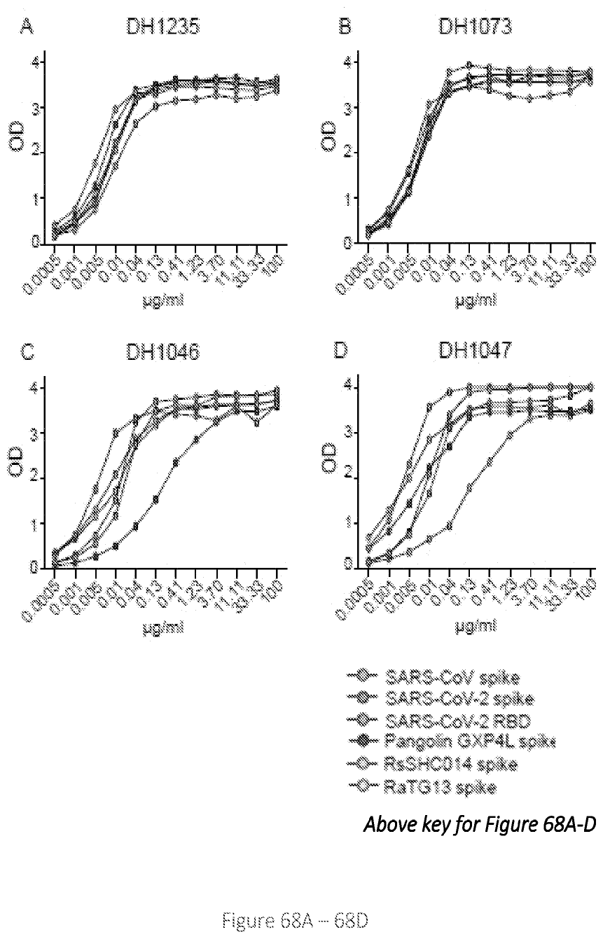

To focus our screen on the four broadly neutralizing mAbs, we measured binding responses for DH1235, DH1073, DH1046, and DH1047 against other zoonotic bat spikes including RaTG13-CoV, bat RsSHC014, and Pangolin GXP4L-CoV spikes. DH1235, DH1073. DH1046, and DH1047 mAbs showed strong binding to bat RaTG13-CoV, bat RsSHC014, and pangolin GXP4L-COV spikes in addition to SARS-COV and SARS-COV-2 (FIG. 68A-68D). DH1235, DH1073, DH1046, and DH1047 bound to SARS-COV-2 RBD and did not bind to the SARS-COV-2 NTD. While DH1235. DH1073, DH1046, and DH1047 were cross-reactive against epidemic, pandemic, and zoonotic sarbecovirus spikes, they did not bind to MERS-COV. HuCoV OC43. HuCoV NL63, or HuCoV 229E spike proteins (FIG. 73), indicating these mAbs recognize a conserved epitope found only in Group 2B betacoronaviruses.

To examine if these cross-reactive mAbs shared any overlap in their epitopes, we examined their binding footprint via both negative stain electron microscopy (NSEM) and surface plasmon resonance (SPR). DH1047 had an overlap with DH1235 but not with DH1073 and had different angles of approach to the SARS-COV-2 spike (FIGS. 74A, 74B, and 74C). Moreover, from SPR competition experiments, DH1047, DH1046, and DH1235 were outcompeted by one another (FIG. 74D), whereas DH1073 was not, indicating that DH1073 targets a distinct non-cross-competing epitope. As DH1047 was the most potent mAb out of the four cross-reactive mAbs in our screen, we also performed NSEM, and observed binding of DH1047 to the RBD of bat RsSHC014 and SARS-COV spike ectodomains, with overall similar orientations as was observed for DH1047 binding to the SARS-COV-2 spike ectodomain (FIG. 75) (16).

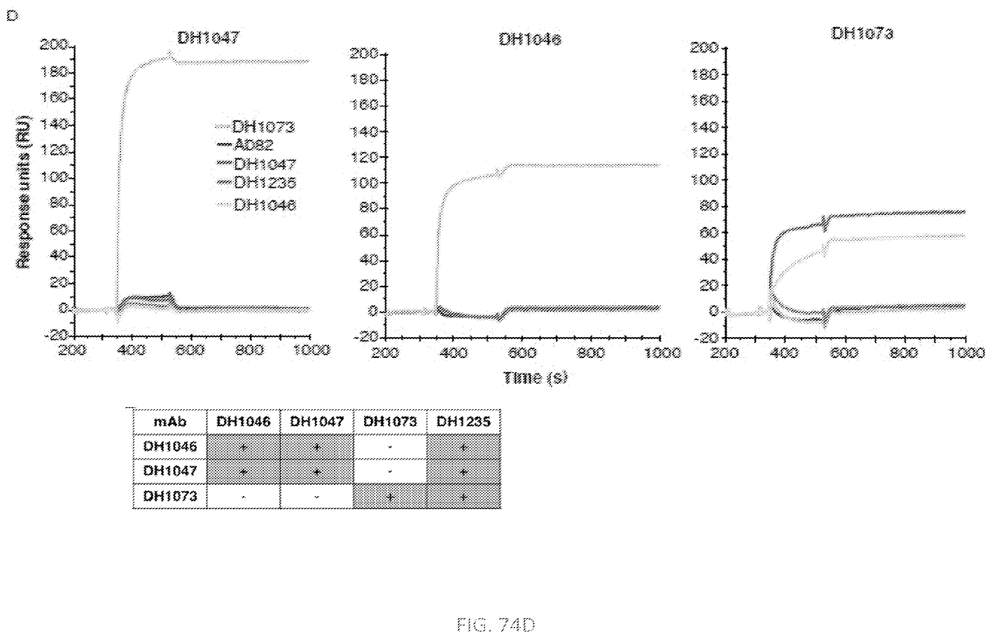

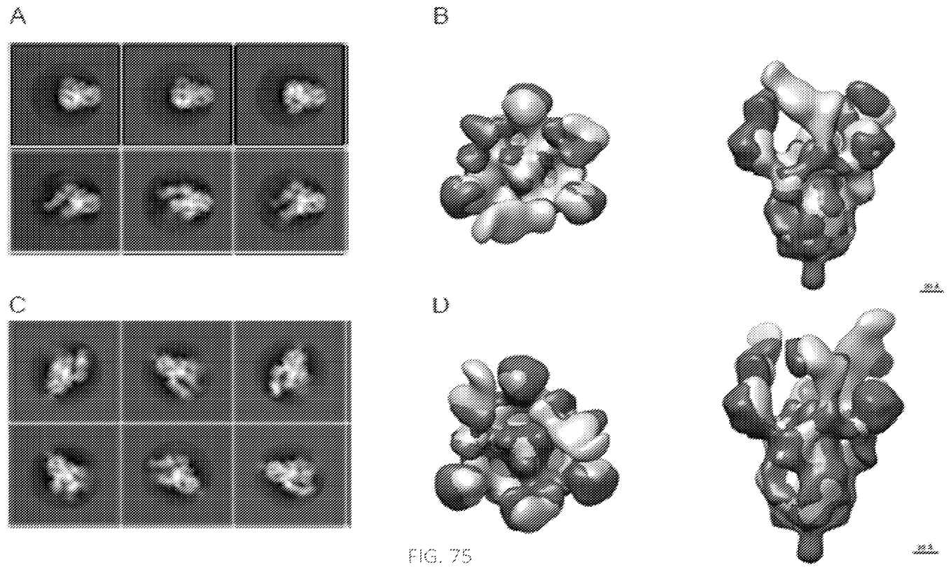

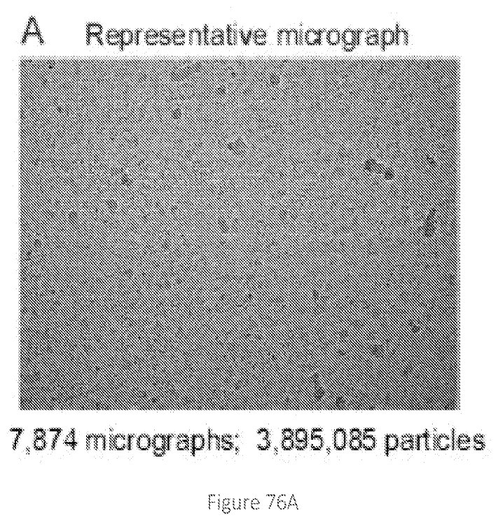

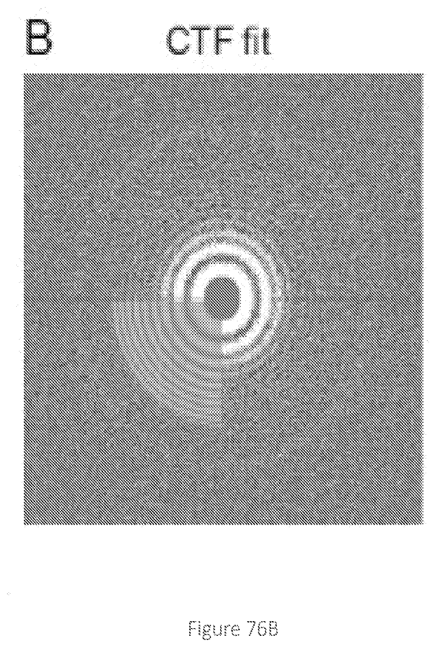

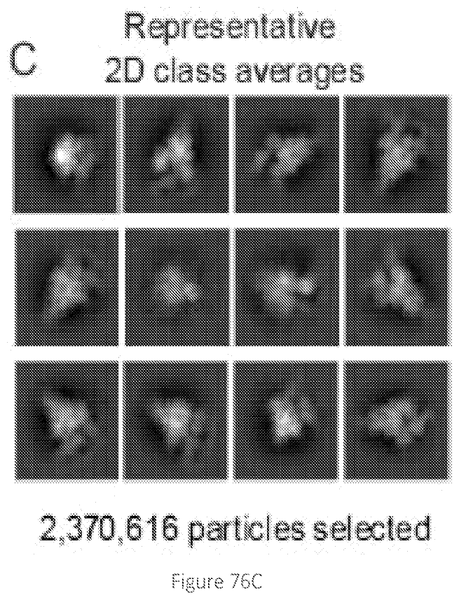

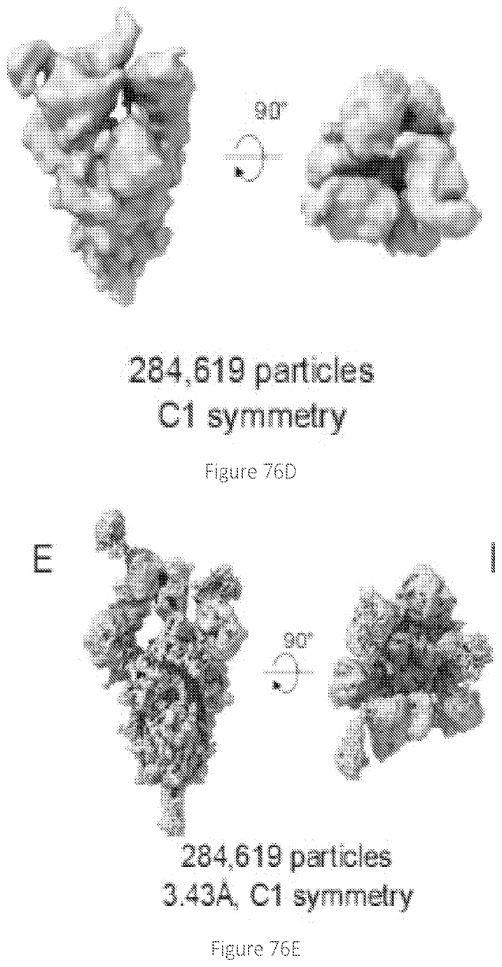

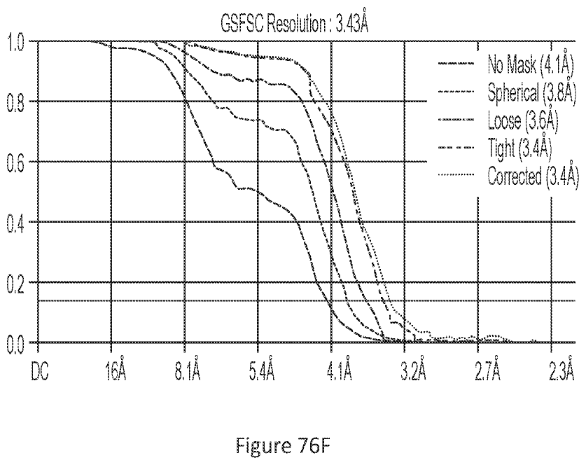

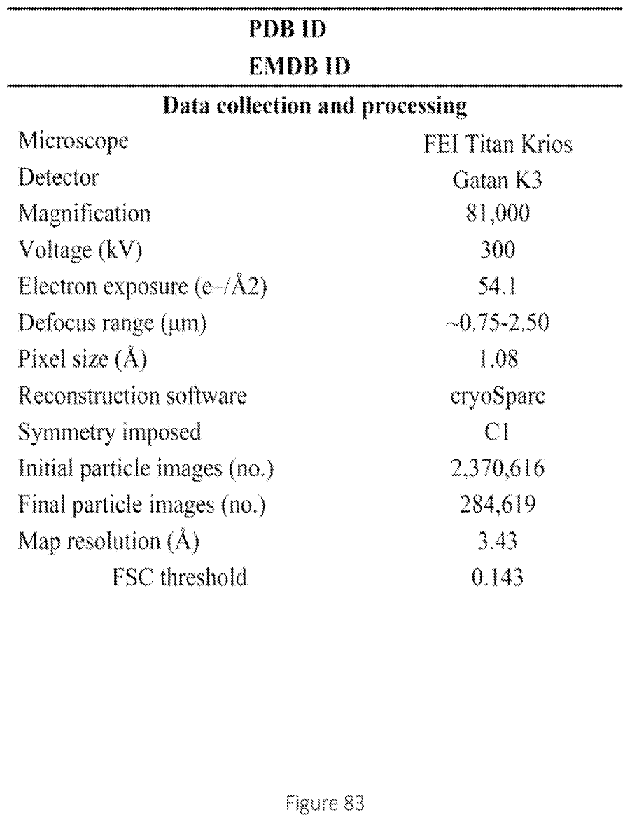

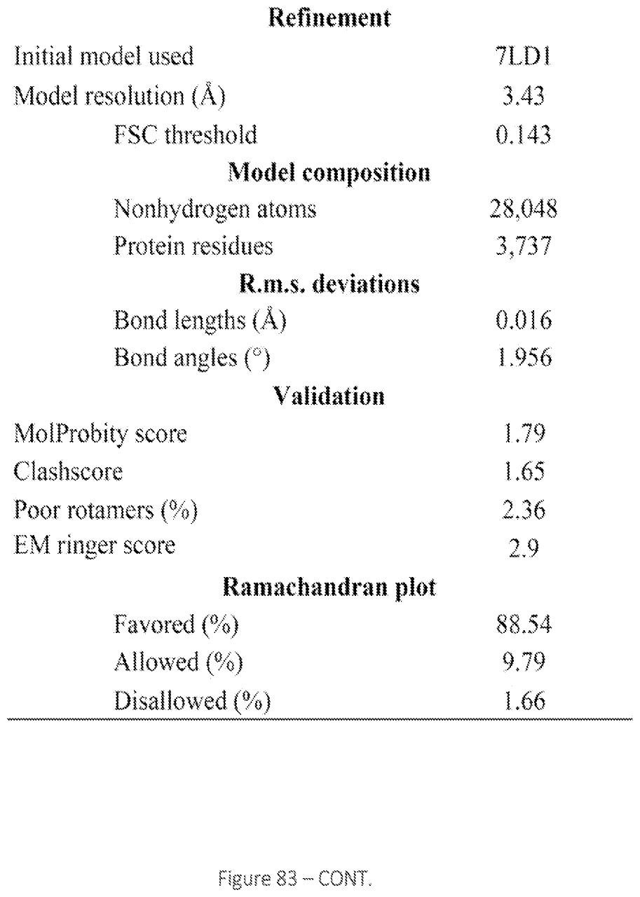

A 3.20 A cryo-EM structure of DH1047 bound to the SARS-COV spike showed three DH1047 Fab bound to each of the 3 RBD of the ectodomain in the “up” position (1 Fab: 1 RBD ratio) (FIG. 68E, FIG. 76 and FIG. 83). DH1047 binding to the SARS-COV RBD involved interactions between the antibody HCDR3 and residues 356-372 of the RBD, and of the LCDR1 and LCDR3 regions with the RBD region spanning residues 390-404. The LCDR3 also interacted with RBM residues 488-492. The angle of approach and footprint of DH1047 on the SARS-COV RBD closely resembled that in the SARS-COV-2 complex (16) with steric overlap predicted with ACE2 binding (FIG. 68E). These results demonstrate that DH1047 binds to SARS-COV and SARS-COV-2 spike ectodomains by involving homologous interactions (FIG. 68F), consistent with our analysis of RBD sequence variability that showed a high degree of convergence of the DH1047 epitope (FIG. 68F) (10), thereby defining an RBD conserved site of vulnerability in sarbecoviruses.

We also defined the binding affinity of DH1047 against epidemic and zoonotic spike proteins. We measured binding on and off rates against both SARS-COV and RsSHC014-CoV spike proteins via SPR. DH1047 bound to the SARS-COV and RsSCH014-COV spikes with high affinity, association rates (>8.60 X10+M-'s-′) and dissociation rates (<1.0X10″ s′″) (FIG. 77), demonstrating that DH1047 binds tightly to both the epidemic SARS-COV and pre-emergent bat CoV spike proteins. Finally, DH1235, DH1073, DH1046, and DH1047 exhibited medium to long heavy-chain-complementarity-determining-region 3 (HCDR3) lengths and variable nucleotide somatic mutation rates in the heavy chain genes. DH1235. DH1073, and DH1046, had HCDR3 lengths of 21, 15, and 24, and nucleotide somatic hypermutation (SMH) rates of 1.7. 9.0, and 4 7, respectively (FIG. 84). The most potent neutralizing antibody DH1047 had HCDR3 lengths and SMH rates of 24 and 8.05, respectively (FIG. 84).

### The Protective Activity of DH1235, DH1073, DH1046, and DH1047 Against SARS-CoV

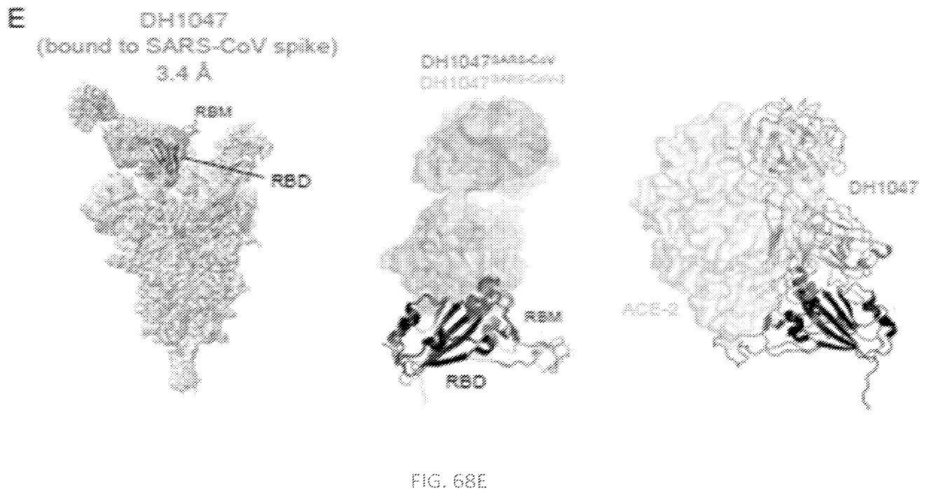

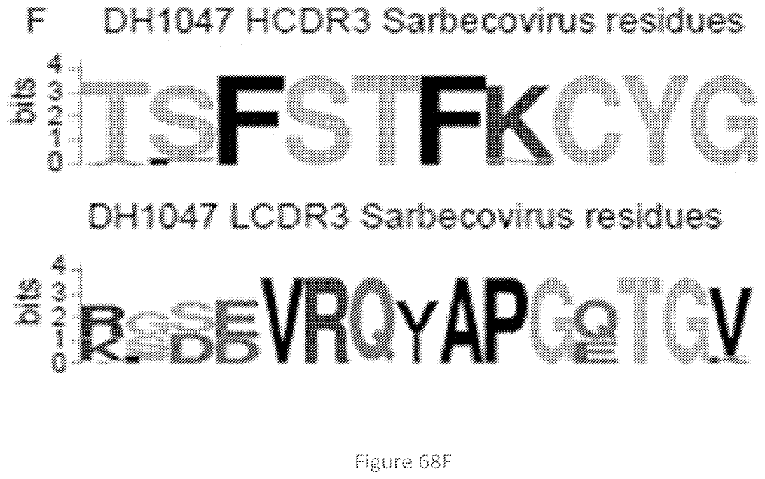

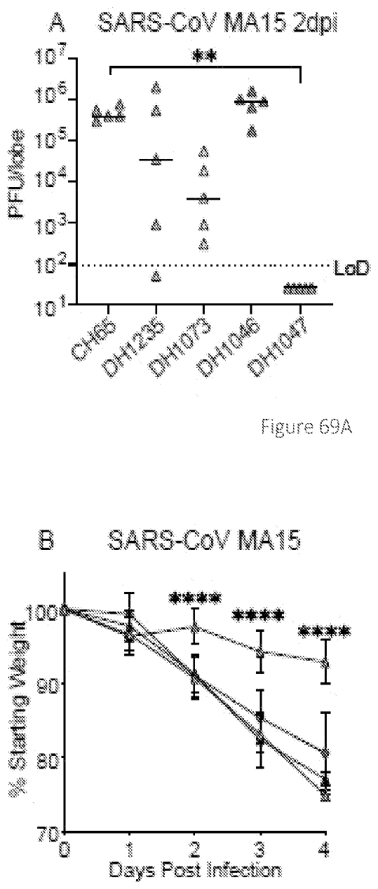

To define the protective efficacy of these four cross-reactive and broadly neutralizing RBD-specific IgG bNAbs, we passively immunized aged mice with DH1235, DH1073, DH1046, DH1047 and a negative control influenza mAb, CH65 (17), at 10 mg/kg 12 hours prior to infection and evaluated lung viral titer replication. Demonstrating a disconnect between in vitro and in vivo protection, neither DH1235, DH1073, nor DH1046 protected against SARS-COV mouse-adapted passage 15 (MA15) challenge in mice and all had lung viral replication comparable to that of control mice (FIG. 69A). In contrast, prophylactic administration of DH1047 fully protected mice from lung viral titer replication (FIG. 69A).

Given the prophylactic potential of DH1047, we sought to also evaluate its therapeutic potential in a highly stringent aged mouse model. We treated mice with control mAb or DH1047 at 10 mg/kg either at 12 hours before or 12 hours post infection with SARS-COV MA15 and monitored mice for signs of clinical disease, including weight loss, pulmonary function, which was measured by whole-body plethysmography (Buxco), through day 4 post infection (4 dpi). In agreement with the SARS-COV MA15 experiments, prophylactic treatment with DH1047 protected mice from weight loss through 4 dpi (FIG. 69B) and lung viral replication (FIG. 69C).

We also evaluated if the prophylactic and therapeutic administration of DH1047 protected against lung pathology as measured by 1) lung discoloration, which is a visual metric of gross lung damage taken at the time of the necropsy, 2) microscopic evaluation as measured by an acute lung injury (ALI) scheme, and 3) a diffuse alveolar damage (DAD) scheme. ALI and DAD, which are characterized by histopathologic changes including alveolar septal thickening, protein exudate in the airspace, hyaline membrane formation, and neutrophils in the interstitium or alveolar sacs, were both blindly evaluated by a board-certified veterinary pathologist blinded to the groups.

The prophylactic administration of DH1047 resulted in complete protection from macroscopic lung discoloration (FIG. 69D) and microscopic lung pathology as measured by ALI (FIG. 69E and FIG. 78) and DAD (FIG. 69F and FIG. 78). Similarly, DH1047 therapy at 12 hours post infection resulted in reduced lung viral titers (FIG. 69C and FIG. 78) as well as the macroscopic lung damage measured by the lung discoloration score (FIG. 69D and FIG. 78). In contrast to the prophylactic treatment condition, therapeutic administration of DH1047 did not significantly reduce microscopic lung pathology compared to control mice as measured by ALI (FIG. 69E and FIG. 78) and DAD (FIG. 69F and FIG. 78) in this highly vulnerable model for SARS-COV pathogenesis. The prophylactic and therapeutic administration of DH1047 prevented degradation of pulmonary function compared to controls (FIG. 69G). Both the prophylactic and therapeutic administration of DH1047 resulted in complete survival whereas only 50% of control mice survived (FIG. 69H). Thus, DH1047 can prevent SARS-COV disease when administered prophylactically and has measurable therapeutic benefits in highly susceptible aged mouse models.

The prophylactic and therapeutic activity of DH1047 against bat pre-emergent CoVs and in vitro neutralization activity against the SARS-COV-2 variants

As DH1047 neutralized both WIV-1 and RsSHC014 (FIG. 67), we sought to define if DH1047 had prophylactic and therapeutic efficacy in mice against these pre-emergent bat CoVs. We administered DH1047 prophylactically 12 hours before infection and therapeutically 12 hours post infection at 10 mg/kg in mice infected with bat CoVs. Importantly, the prophylactic administration of DH1047 completely protected mice from WIV-1 lung viral replication and reduced lung viral titers in therapeutically treated mice compared to control mice (FIG. 70A).

Similarly, the prophylactic administration of DH1047 completely protected mice from RsSHC014 lung viral replication and significantly reduced viral replication to near undetectable levels in therapeutically treated mice (FIG. 70B). While we previously demonstrated the prophylactic and therapeutic efficacy of DH1047 against the SARS-COV-2 Wuhan isolate in cynomolgus macaques (16), which exhibit mild SARS-COV-2 disease (18), it was not known if the mutations present in the newly emerging SARS-COV-2 variants will ablate the neutralizing activity of DH1047. We therefore evaluated if DH1047 can neutralize the prevalent variants of concern (VOCs) using both pseudovirus and live virus neutralization assays. DH1047 neutralized all tested variants of concern including SARS-COV-2 D614G, B.1.1.7 (Alpha), B.1.351 (Beta), P.1 (Gamma), B.1.429, B.1.526, B.1.617.1 (Kappa), and B.1.617.2 (Delta) (FIG. 70C and FIG. 70D). Impressively, the PsVNA50 values against all the variants ranged between 0.1214-0.1609 mg/ml confirming across the board and demonstrated neutralization of SARS-COV-2 VOCs by mAb DH1047 (FIG. 70D). Live virus neutralization also demonstrated the broadly neutralizing activity of DH1047 with IC50 values against D614G, B.1.1.7, and B.1.351 were 0.059, 0.081, and 0.111 ug/ml, respectively.

### The Prophylactic and Therapeutic Activity of DH1047 Against SARS-COV-2 B.1.351 in Mice

Given that the B.1.351 variant is more resistant to both vaccine-elicited neutralizing antibodies (13. 19), and completely ablates the neutralizing activity of the Eli Lily therapeutic monoclonal antibody LY-CoV555 (II), we also sought to evaluate if DH1047 had both prophylactic and therapeutic efficacy against SARS-COV-2 B.1.351 in mice. We again utilized a highly susceptible and vulnerable aged mouse model in the SARS-COV-2 B.1.351 protection experiments. Consistent with the SARS-COV, WIV-1, and RsSHC014 in vivo data, the prophylactic administration of DH1047 mediated complete protection against severe weight loss following SARS-COV-2 B.1.351 challenge in aged mice (FIG. 71A).

In contrast, we did not observe differences in weight loss after the therapeutic administration of DH1047 as compared to controls (FIG. 71A). Mice prophylactically treated with DH1047 had undetectable levels of SARS-COV-2 B.1.351 lung viral replication (FIG. 71B) and were also completely protected from macroscopic lung pathology compared to controls (FIG. 71C). Therapeutic administration of DH1047 resulted in a significant reduction in lung viral titers compared to control (FIG. 71B). We also evaluated the microscopic lung pathology as measured by ALI (FIG. 71D) and DAD scoring schemes (FIG. 71E) in this highly susceptible aged model for SARS-COV-2 B.1.351 pathogenesis. Importantly, the prophylactic administration of DH1047 significantly protected mice from lung histopathology as measured by ALI and DAD compared to control mice.

Additionally, we observed a reduction in ALI by the therapeutic administration of DH1047 as measure by macroscopic lung pathology (FIG. 71C) and lung histopathology by ALI (FIG. 71D). Finally, there was no difference in mortality in the SARS-COV-2 B.1.351 challenge model in the control and DH1047-treated mice (FIG. 71F). Therefore, DH1047 can prevent and treat SARS-COV-2 infections with the B.1.351 variant of concern in vivo. To determine the concentration at which we observe breakthrough infection in vivo we performed a dose de-escalation study and treated mice prophylactically at 10, 5, and 1 mg/kg. We observed full protection at 10 but not at 5 mg/kg as measured by weight loss and lung discoloration, despite full protection lung viral replication compared to control (FIG. 71G, 71J, 711). In contrast, incomplete protection from weight loss, breakthrough viral lung replication, and lung pathology was observed at the 1 mg/kg prophylaxis dose of DH1047 (FIG. 71G, 71J, 71I).

### Structural Comparison of DH1047 to Other Antibodies Targeting the RBD

Comparing the DH1047 epitope with that of representative RBD-directed antibodies from Class 1, 2, 3 and 4 (20), we found that the DH1047 footprint overlapped with antibody C105 (Class 1) and CR3022 (Class 4) (FIG. 71J and FIG. 79). The DH1047 footprint on the RBD showed similarity with Ab H014 (27) and overlapped with the footprint of other antibodies whose epitopes overlap with that of CR3022 (22-24). Interestingly, single domain antibodies, or nanobodies, that potently neutralize SARS-COV-2 show a close overlap with the DH1047 VH domain (FIG. 79).

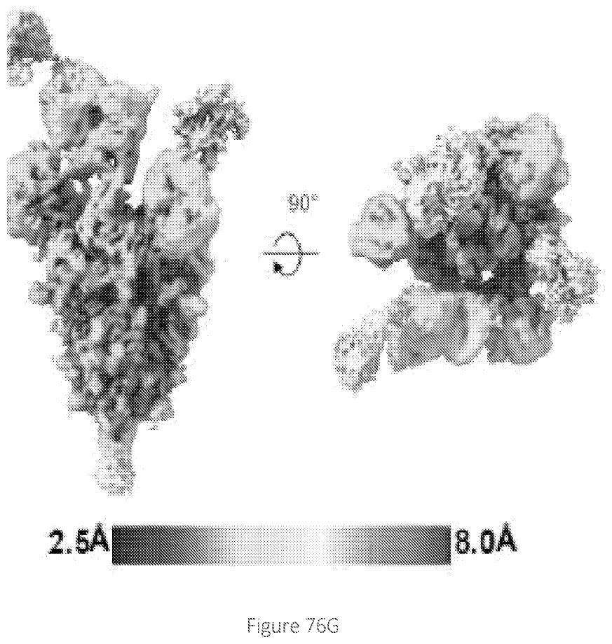

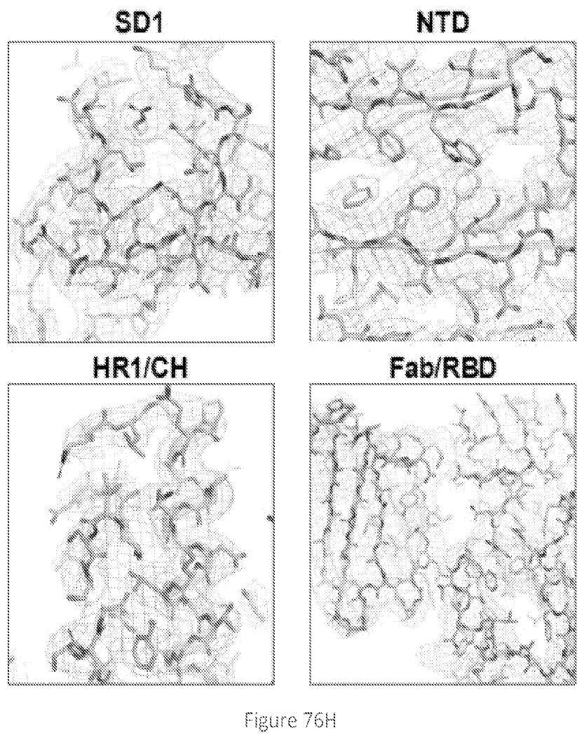

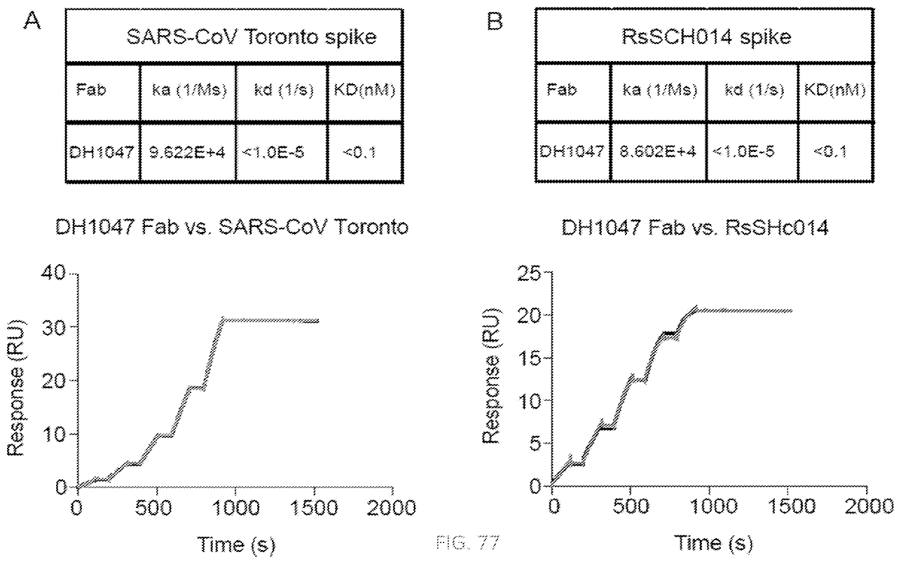

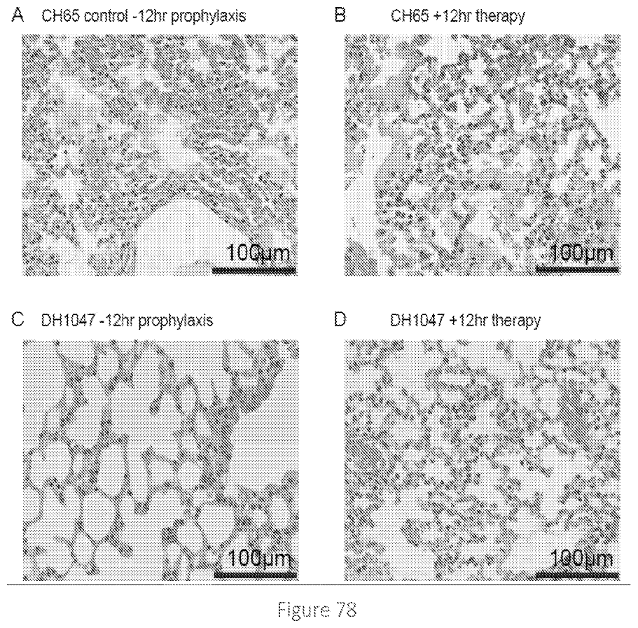

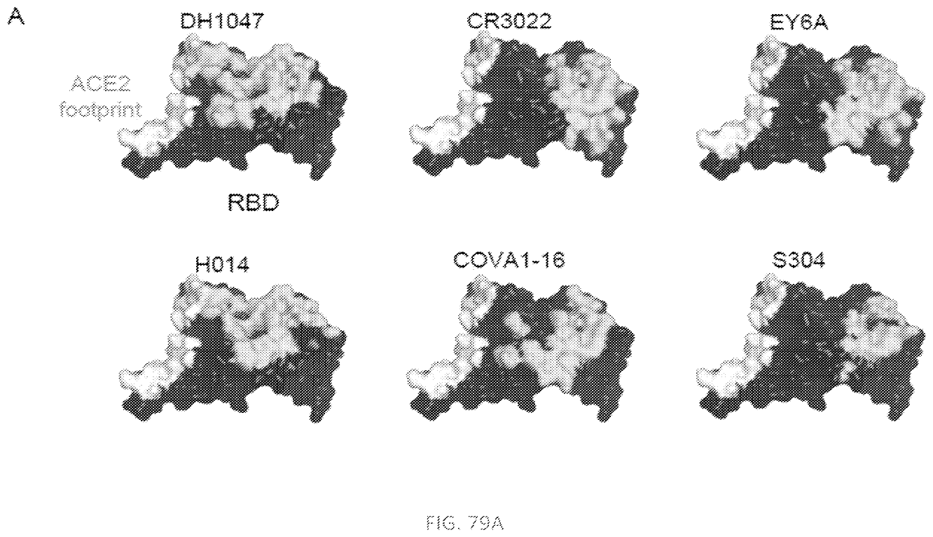

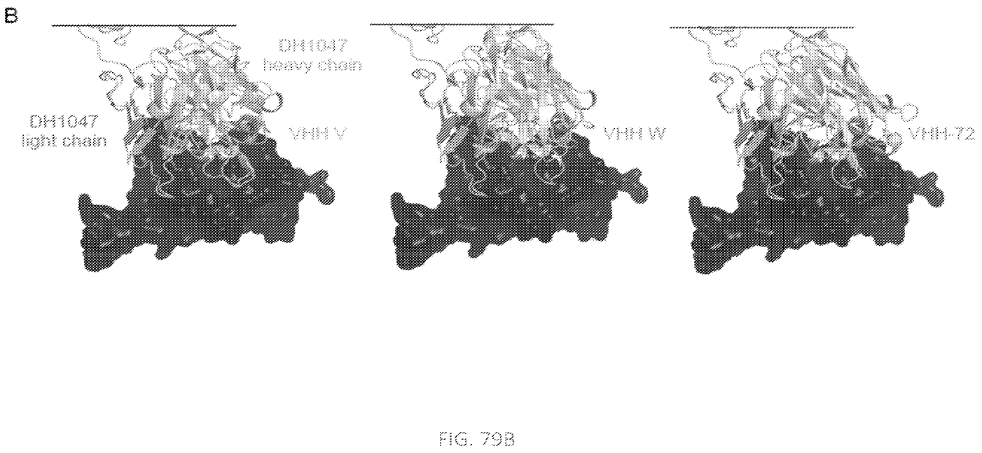

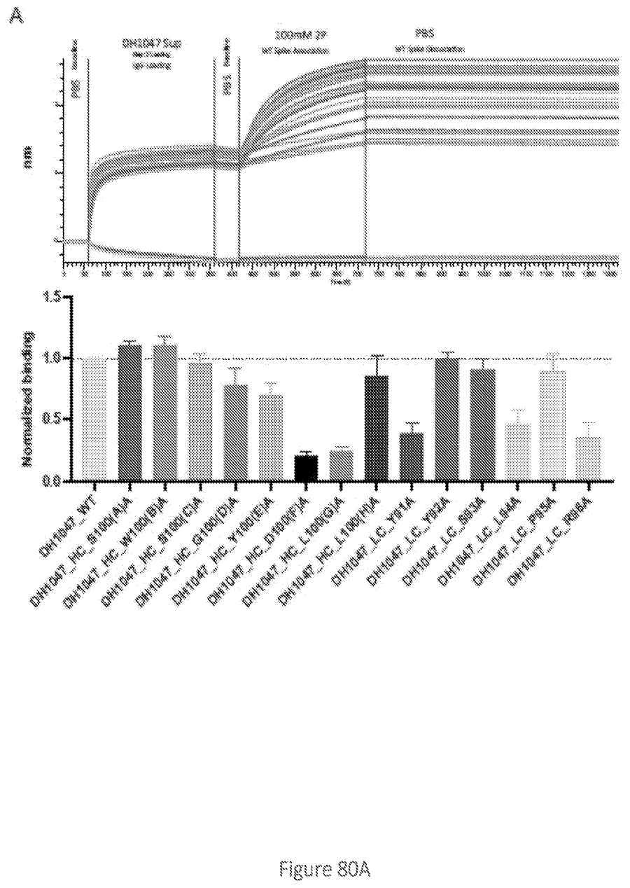

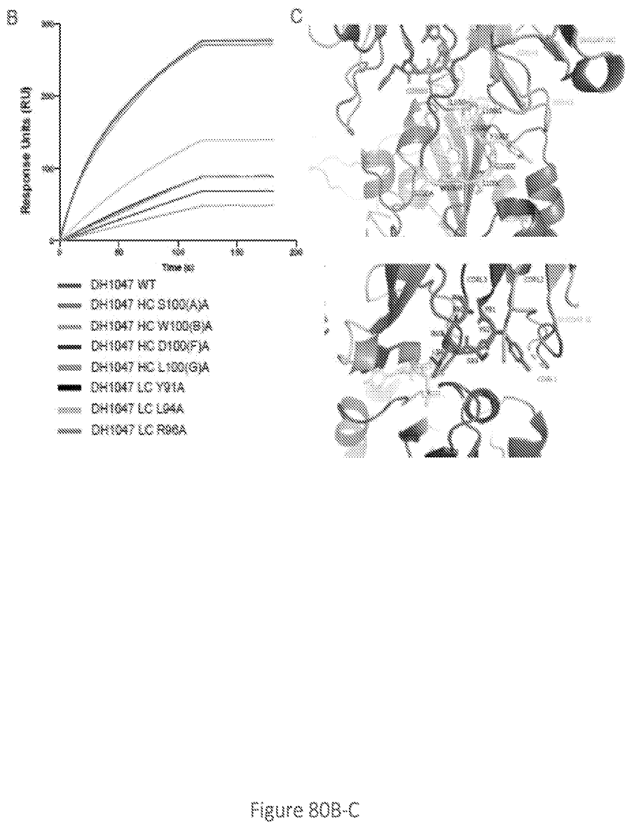

Nanobody VHH V shows the closest overlap with its elongated HCDR3 loop binding to a similar RBD region as the HCDR3 of DH1047. We performed alanine scanning mutagenesis of the DH1047 HCDR3 residues proximal to the RBD epitope (FIG. 80). Mutating residue Trp 100B to Ala did not alter its binding to the SARS-COV-2 2P S ectodomain, consistent with the orientation of the Trp side chain away from the epitope. Similarly, mutating residues Ser100A and Ser100C to Ala did not alter binding. Ala mutants of residues Leu 100G and Asp 10OF notably reduced DH1047 binding to the S protein even though these residues did not directly contact the RBD, without wishing to be bound by theory disrupting the conformation of the long HCDR3 loop. Mutagenesis of the LCDR3 similarly resulted in notable reduction in binding observed for Tyr91Ala and Arg96Ala substitutions, although neither of these residues directly contacted the RBD. Reduction in binding was also observed for Ala substitution of residue Leu94 where the Leu side chain occupied a hydrophobic pocket formed by the heavy chain residues Leu100G and Ile58, and RBD residue Val394.

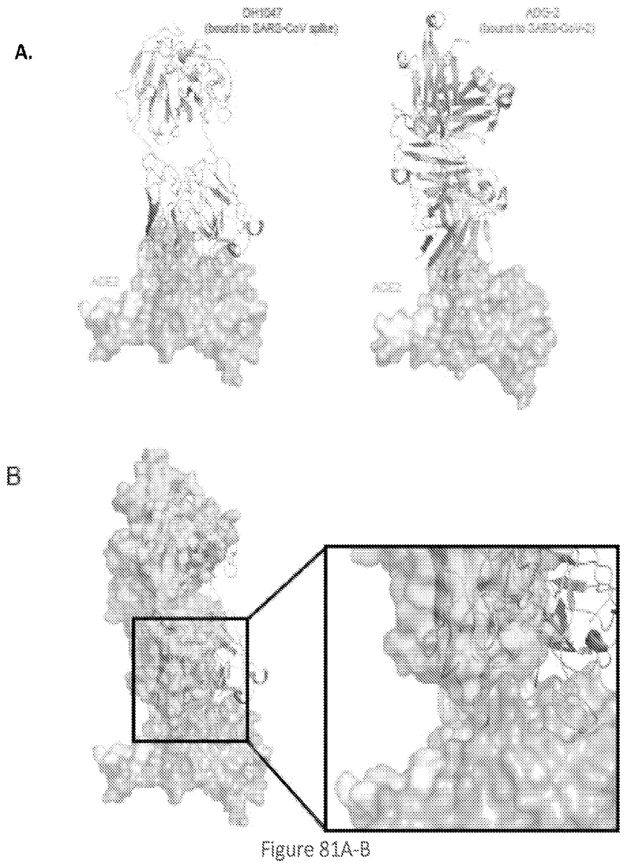

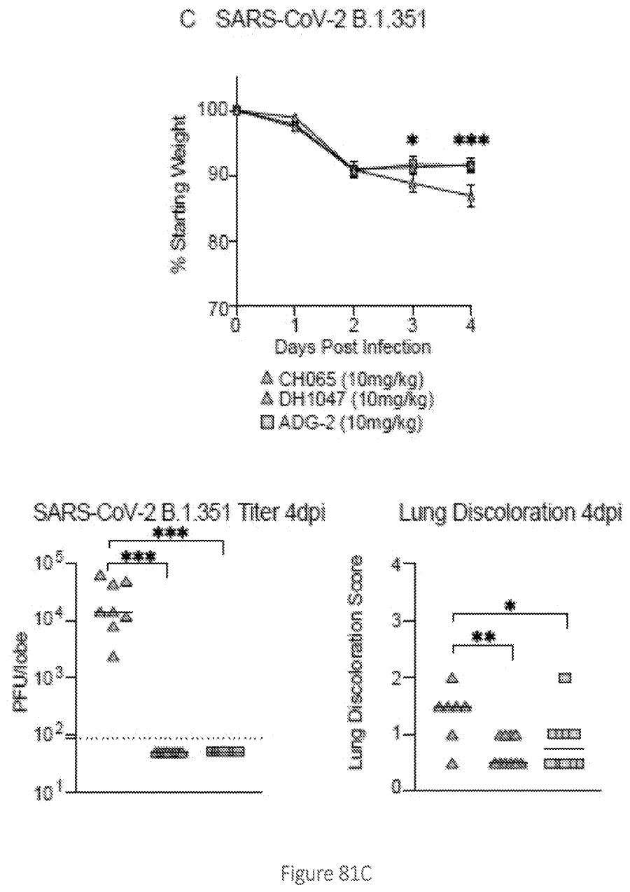

The DH1047 epitope also overlaps with that of antibody ADG-2 previously shown to neutralize SARS-related viruses and protecting against SARS-COV and SARS-COV-2 (15). The approach angles of DH1047 and ADG-2 differ, with a rotation about the Fab longitudinal axis pivoting the ADG-2 antibody more towards the ACE2 binding region compared to DH1047 (FIGS. 81A and 81B). In contrast to ADG-2 which uses VH3-21 for its heavy chain and has a 17 amino acid long HCDR3, DH1047 uses VH1-46 and has a 24 amino acid long HCDR3 (FIG. 84) (15). To compare the therapeutic efficacy of DH1047 against ADG-2, we performed a head-to-head prophylaxis and therapy comparison study. Aged mice were therapeutically treated with either DH1047, ADG-2, or CH65 control mAbs at 10 mg/kg at 6 hours post infection with SARS-COV-2 B.1.351. Mice treated with either DH1047 or ADG-2 were completely protected from weight loss, lung viral replication, and lung damage compared to controls (FIG. 81C), indicating that DH1047 has similar in vivo therapeutic profiles to ADG-2, which is another potent cross-protective mAb (15).

### DISCUSSION

The emergence of SARS-COV and SARS-COV-2 in the last two decades underscores a critical need to develop broadly effective countermeasures against sarbecoviruses. Moreover, with the recent emergence of the more transmissible (25), modestly more virulent (26) B. 1.1.7 variant (Alpha), B.1.351 (Beta), B.1.1.28 (Gamma), and the highly transmissible B.1.617.2 variant (Delta), all of which can partially evade existing countermeasures (11. 13. 27), there is a need to develop next-generation mAb therapeutics that can broadly neutralize these variants, as well as future variants of concern. For example, the SARS-COV-2 B.1.351 variant completely ablates the neutralization activity of the mAb LY-CoV555 (11. 12). As a result, the emergency use authorization (EUA) of LY-CoV555 was recently rescinded by the U.S. Food and Drug Administration (FDA). In addition, the presence of the E484K mutation in many variants of concern, severely dampens the neutralization activity by more than 6-fold of the AstraZeneca COV2-2196 mAb, Brii BioSciences mAb Brii-198, and the Regeneron mAb REGN 10933 (12. 13).

In addition to evading currently monoclonal antibody therapeutics, some of the variants including B. 1.351 can diminish the efficacy of clinically approved vaccines, including the Johnson & Johnson single-dose vaccine and the AstraZeneca ChAd0×1 (28. 29). Furthermore, some monoclonal antibodies isolated from vaccine recipients of the Moderna and Pfizer vaccines also demonstrated reduced efficacy against mutations present in the variants (30). Therefore, current vaccine and mAb therapies must be monitored in real time to define the performance of existing therapies against newly emerging and spreading variants. In the setting of reduced vaccine efficacy, the deployment of effective mAb therapies against the variants, such as DH1047, can be a strategy to help control the COVID-19 pandemic.

The development of universal vaccination strategies against sarbecoviruses will be improved by the identification and characterization of broadly protective and conserved epitopes across SARS-related virus strains. Recent studies described broadly reactive antibodies that target the subunit 2 (S2) portion of the spike protein (31-34). While the broad recognition of these S2-specific antibodies is encouraging, these antibodies weakly neutralized diverse CoVs. Given the limited characterization of these mAbs, it is unclear if these S2-specific mAbs are broadly protective in vivo against diverse epidemic and zoonotic pre-emergent CoVs. In contrast, RBD-specific antibody, S2X259, neutralized SARS-COV-2 variants and zoonotic SARS-related viruses, as measured by pseudovirus neutralization (35).

Similarly, a recent subset of RBD-specific cross-reactive mAbs also showed in vitro activity (36), although their in vivo breadth and protective efficacy against divergent sarbecoviruses remains unconfirmed. In contrast to DH1047, neither DH1235, DH1073, nor DH1046 fully protected against SARS-COV infection in aged mice, without wishing to be bound by theory, because these cross-reactive mAbs may have differences in their mechanisms of protection in vivo. While it is not clear why these mAbs have in vivo protection differences, without wishing to be bound by theory, they have differences in their angle of approach to the spike and/or require optimal Fc effector functions or may have stability differences in vivo.

DH1047 had broad protective in vivo efficacy against pre-emergent SARS-related viruses, epidemic SARS-COV, SARS-COV-2 B.1.351 variant, and neutralized all tested SARS-CoV-2 variants including the delta variant, underscoring that DH1047 recognizes a pan Sarbecovirus neutralizing epitope. Consistent with this notion, we have described a SARS-CoV-2 RBD-ferritin nanoparticle vaccine that elicited neutralizing antibodies against pre-emergent SARS-related viruses and protected against SARS-COV-2 challenge in monkeys (10). The serum antibody responses in these SARS-COV-2 RBD-ferritin nanoparticle-vaccinated monkeys can block DH1047 binding responses against SARS-COV-2 spike proteins, indicating that SARS-COV-2 RBD vaccines elicit DH1047-related antibody responses and can potentially protect against the future emergence of SARS- or SARS2-related viruses. As with other broad Sarbecovirus vaccine formulations and a recent study which reported broad neutralization of sarbecoviruses in SARS convalescent patients fully immunized with SARS-COV-2 mRNA vaccines (8. 37), and it will be interesting to examine if these vaccines and individuals generate DH1047-like neutralizing antibodies.

Moving forward, it will be critical to closely monitor SARS- and SARS2-related viruses of zoonotic origin and actively monitor if broad-spectrum antibodies like ADG-2, DH1047, and S2X259 retain their inhibitory activity against pre-emergent viruses. Embodiments comprise a system in which broad-spectrum antibodies like DH1047 can be tested for safety in small Phase I clinical trials so that if a future SARS-related virus emerges, DH1047 can immediately be tested in larger efficacy trials at the site of an outbreak to potentially prevent the rapid spread of an emergent CoV. Moreover, given that DH1047 neutralized all tested SARS-COV-2 variants including the highly transmissible B.1.617.2 (Delta), this mAb can also be deployed now to help curb the COVID-19 pandemic. Like other therapeutic antibodies evaluated against COVID19 infections, our data argue that early administration will prove critical for protecting against severe disease outcomes (38). We conclude that DH1047 is a broadly protective mAb that has efficacy against pre-emergent, zoonotic SARS-related viruses from different clades, neutralizes highly transmissible SARS-CoV-2 VOCs, and protects against SARS-COV-2 B. 1.351.

### Methods

### Antibody Isolation

Antibodies were isolated from antigen-specific single B cells as previously described from an individual who had recovered from SARS-COV-1 infection 17 years prior to leukapheresis, and from a SARS-COV-2 convalescent individual from 36 days post infection (16).

### Measurement of CoV Spike Binding by ELISA

Indirect binding ELISAs were conducted in 384 well ELISA plates (Costar #3700) coated with 2 ug/ml antigen in 0.1M sodium bicarbonate overnight at 4° C., washed and blocked with assay diluent (1XPBS containing 4% (w/v) whey protein/15% Normal Goat Serum/0.5% Tween-20/0.05% Sodium Azide), mAbs were incubated for 60 minutes in three-fold serial dilutions beginning at 100 ug/ml followed by washing with PBS/0.1% Tween-20. HRP conjugated goat anti-mouse IgG secondary antibody (SouthernBiotech 1030-05) was diluted to 1:10,000 in assay diluent without azide, incubated at for 1 hour at room temperature, washed and detected with 20 μl SureBlue Reserve (KPL 53-00-03) for 15 minutes. Reactions were stopped via the addition of 20 μl HCL stop solution. Plates were read at 450 nm. Area under the curve (AUC) measurements were determined from binding of serial dilutions.

### Measurement of Neutralizing Antibodies Against Live Viruses

Full-length SARS-COV-2 Seattle. SARS-COV-2 D614G. SARS-COV-2 B.1.351. SARS-COV-2 B.1.1.7. SARS-COV. WIV-1, and RsSHC014 viruses were designed to express nanoluciferase (nLuc) (8). Virus titers were measured in Vero E6 USAMRIID cells, as defined by plaque forming units (PFU) per ml, in a 6-well plate format in quadruplicate biological replicates for accuracy. For the 96-well neutralization assay. Vero E6 USAMRID cells were plated at 20.000 cells per well the day prior in clear bottom black walled plates. Cells were inspected to ensure confluency on the day of assay, mAbs were serially diluted 3-fold up to nine dilution spots at specified concentrations. Serially diluted mAbs were mixed in equal volume with diluted virus. Antibody-virus and virus only mixtures were then incubated at 37° C. with 5% CO₂ for one hour. Following incubation, serially diluted mAbs and virus only controls were added in duplicate to the cells at 75 PFU at 37° C., with 5% CO₂. After 24 hours, cells were lysed, and luciferase activity was measured via Nano-Glo Luciferase Assay System (Promega) according to the manufacturer specifications. Luminescence was measured by a Spectramax M3 plate reader (Molecular Devices. San Jose, CA). Virus neutralization titers were defined as the sample dilution at which a 50% reduction in RLU was observed relative to the average of the virus control wells.

### Lentivirus Pseudovirus Neutralization Assay (PsVNA)

Antibody preparations were evaluated by SARS-COV-2 pseudovirus 50% neutralization assay (PsVNA50)) using WA-1 strain. B.1.1.7 (with spike mutations: H69-V70del. Y144del. N501Y. A570D. D614G. P681H. T716I. S982 Å, and D1118H). B. 1.429 (with spike mutations S13I. W152C. L452R. D614G). B.1.526 (with spike mutations L5F. T95I. D253G. E484K or S477N. D614G. A701V). P.1 (with spike mutations L18F. T20N. P26S. D138Y. R190S. K417T. E484K. N501Y. H655Y. T1027I. D614G. V1176F). B.1.351 (with spike mutations L18F. D80 Å. D215G. L242-244del. K417N. E484K. N501Y. D614G, and A701V). B.1.617.1 (with spike mutations G142D. E154K. V382L. L452R. E484Q. P681R. Q1071H. D1153Y) and B. 1.617.2 (with spike mutations T19R. G142D. E156del. F157del. R158G, L452R. T478K. D614G. P681R. D950N). The PsVNA using 293-ACE2-TMPRSS2 cell line was described previously (39. 40). Controls included cells only, virus without any antibody and positive sera. The cut-off value or the limit of detection for the neutralization assay is 1:10 dilution.

### Surface Plasmon Resonance

Kinetic measurements of the DH1047 Fab binding to SARS-COV and RsSHC014 spike proteins were obtained using a Biacore S200 instrument (Cytiva, formerly GE Healthcare) in HBS-EP+1× running buffer. The spike proteins were first captured onto a Series S Streptavidin chip to a level of 300-400 for the SARS-COV spike proteins and 850-1000RU for the RsSHC014 spike protein. The DH1047 Fab was diluted from 2.5 to 200 nM and injected over the captured CoV spike proteins using the single cycle kinetics injection type at a flow rate of 50 μL/min. There were five 120s injections of the Fab at increasing concentrations followed by a dissociation of 600s after the final injection. After dissociation, the spike proteins were regenerated from the streptavidin surface using a 30s pulse of Glycine pH1.5. Results were analyzed using the Biacore S200 Evaluation software (Cytiva). A blank streptavidin surface along with blank buffer binding were used for double reference subtraction to account for non-specific protein binding and signal drift. Subsequent curve fitting analyses were performed using a 1:1 Langmuir model with a local Rmax for the DH1047 Fab. The reported binding curves are representative of 2 data sets.

### Protein Expression and Purification for EM Studies

The SARS-COV spike ectodomain construct comprised the residues 1 to 1190 (UniProt P59594-1) with proline substitutions at 968-969, a C-terminal T4 fibritin trimerization motif, a C-terminal HRV3C protease cleavage site, a TwinStrepTag and an 8XHisTag. The construct was cloned into the mammalian expression vector paH (41). The RsSHC014 spike ectodomain construct was prepared similarly, except it also contained the 2P mutations that placed two consecutive proline at the HRI-CH junction at residue positions 986 and 987. FreeStyle 293F cells were used for the spike ectodomain production. Cells were maintained in FreeStyle 293 Expression Medium (Gibco) at 37° C., and 9% CO₂, with agitation at 120 rpm in a 75% humidified atmosphere. Transfections were performed as previously described (42. 43) using Turbo293 (SpeedBiosystems). 16 to 18 hours post transfection, HyClone CDM4HEK293 media (Cytiva. MA) was added. On the 6th day post transfection, spike ectodomain was harvested from the concentrated supernatant. The purification was performed using StrepTactin resin (IBA LifeSciences) and size exclusion chromatography (SEC) on a Superose 6 10/300 GL Increase column (Cytiva, MA) in 2 mM Tris, pH 8.0, 200 mM NaCl, 0.02% NaN3. All steps were performed at room temperature and the purified spike proteins were concentrated to 1-5 mg/ml, flash frozen in liquid nitrogen and stored at −80° C., until further use.

DH1047 IgG was produced in Expi293F cells maintained in Expi293 Expression Medium (Gibco) at 37° C., 120 rpm, 8% CO₂ and 75% humidity. Plasmids were transfected using the ExpiFectamine 293 Transfection Kit and protocol (Gibco) (42. 44) and purified by Protein A affinity. The IgG was digested to the Fab state using LysC.

### Negative Stain Electron Microscopy (NSEM)

NSEM was performed as described previously (16). Briefly, Fab-spike complexes were prepared by mixing Fab and spike to give a 9: Imolar ratio of Fab to spike. Following a 1-hr incubation for 1 hour at 37° C., the complex was cross-linked by diluting to a final spike concentration of 0.1 mg/ml into room-temperature buffer containing 150 mM NaCl, 20 mM HEPES pH 7.4, 5% glycerol, and 7.5 mM glutaraldehyde and incubating for 5 minutes. Excess glutaraldehyde was quenched by adding sufficient 1 M Tris pH 7.4 stock to give a final concentration of 75 mM Tris and incubated for 5 minutes. Carbon-coated grids (EMS, CF300-cu-UL) were glow-discharged for 20s at 15 mA, after which a 5-μl drop of quenched sample was incubated on the grid for 10-15 s, blotted, and then stained with 2% uranyl formate. After air drying grids were imaged with a Philips EM420 electron microscope operated at 120 KV, at 82,000× magnification and images captured with a 2k x 2k CCD camera at a pixel size of 4.02 λ.

The RELION 3.0 program was used for all negative stain image processing Images were imported, CTF-corrected with CTFFIND, and particles were picked using a spike template from previous 2D class averages of spike alone. Extracted particle stacks were subjected to 2-3 rounds of 2D class averaging and selection to discard junk particles and background picks. Cleaned particle stacks were then subjected to 3D classification using a starting model created from a bare spike model, PDB 6vsb, low-pass filtered to 30 Å. Classes that showed clearly defined Fabs were selected for final refinements followed by automatic filtering and B-factor sharpening with the default Relion post-processing parameters.

### Cryo-EM

Purified SARS-COV-1 spike ectodomain was incubated for approximatively 2 hours with a 6-fold molar equivalent of the DH1047 Fab in a final volume of 10 μL. The sample concentration was adjusted to ˜1.5 mg/mL of spike in 2 mM Tris pH 8.0, 200 mM NaCl, and 0.02% NaN3. Before freezing, 0.1 μL of glycerol was added to the 10 μL of sample. A 2.4-uL drop of protein was deposited on a Quantifoil-1.2/1.3 grid (Electron Microscopy Sciences, PA) that had been glow discharged for 10 seconds using a PELCO easiGlow™ Glow Discharge Cleaning System. After a 30-second incubation in >95% humidity, excess protein was blotted away for 2.5 seconds before being plunge frozen into liquid ethane using a Leica EM GP2 plunge freezer (Leica Microsystems). Frozen grids were imaged using a Titan Krios (Thermo Fisher) equipped with a K3 detector (Gatan). Data processing was performed using cryoSPARC (45). Model building and refinement was done using Phenix (46. 47), Coot (48), Pymol (49), Chimera (50), ChimeraX (51) and Isolde (52). Similar to what we had observed for the DH1047 complex with the SARS-COV-2 spike ectodomain (16), there was considerable heterogeneity in the RBD region: further classification of particles was performed to better resolve the antibody binding interface, resulting in an asymmetric reconstruction of a population refined to a resolution of 3.4 A that was used for model fitting.

### Animals and Challenge Viruses

Eleven-month-old female BALB/c mice were purchased from Envigo (#047) and were used for the SARS-COV, SARS-COV-2 B.1.351, and RsSHC014-COV protection experiments. 8-10-week-old hACE2-transgenic mice (B6J.Cg-Tg (FOXJI-ACE2) | Rba/Mmnc) were bred at UNC Chapel Hill and were used for WIV-1-COV protection experiments. The study was carried out in accordance with the recommendations for care and use of animals by the Office of Laboratory Animal Welfare (OLAW), National Institutes of Health and the Institutional Animal Care and Use Committee (IACUC) of University of North Carolina (UNC permit no. A-3410-01). Animals were housed in groups of five and fed standard chow diets. Virus inoculations were performed under anesthesia and all efforts were made to minimize animal suffering. All mice were anesthetized and infected intranasally with 1×10⁴ PFU/ml of SARS-CoV MA15, 1×10⁴ PFU/ml of SARS-COV-2 B.1.351-MA10, 1 × 10+PFU/ml RsSHC014, 1×10⁴ PFU/ml WIV-1, which have been described previously (6. 53. 54). Mice were weighted daily and monitored for signs of clinical disease, and selected groups were subjected to daily whole-body plethysmography. For all mouse studies, groups of n=10 mice were included per arm of the study except for the hACE2-transgenic mice, which included n=5 mice per group due to a limited availability of these mice. Viral titers, weight loss, and histology were measured from individual mice per group.

### Lung Pathology Scoring

Acute lung injury was quantified via two separate lung pathology scoring scales: Matute-Bello and Diffuse Alveolar Damage (DAD) scoring systems. Analyses and scoring were performed by a board certified veterinary pathologist who was blinded to the treatment groups as described previously (55). Lung pathology slides were read and scored at 600× total magnification.

The lung injury scoring system used is from the American Thoracic Society (Matute-Bello) in order to help quantitate histological features of ALI observed in mouse models to relate this injury to human settings. In a blinded manner, three random fields of lung tissue were chosen and scored for the following: (A) neutrophils in the alveolar space (none=0), 1-5 cells =1, >5 cells =2), (B) neutrophils in the interstitial septa (none=0), 1-5 cells =1, >5 cells =2), (C) hyaline membranes (none=( ) one membrane=1, >1 membrane=2), (D) Proteinaceous debris in air spaces (none=( ) one instance=1, >1 instance=2), (E) alveolar septal thickening (<2× mock thickness=0), 2-4× mock thickness=1, >4× mock thickness=2). To obtain a lung injury score per field, A-E scores were put into the following formula

score=[(20×A)+(14×B)+(7×C)+(7×D)+(2×E)]/100.

This formula contains multipliers that assign varying levels of importance for each phenotype of the disease state. The scores for the three fields per mouse were averaged to obtain a final score ranging from ( ) to and including 1. The second histology scoring scale to quantify acute lung injury was adopted from a lung pathology scoring system from lung RSV infection in mice (56). This lung histology scoring scale measures diffuse alveolar damage (DAD). Similar to the implementation of the ATS histology scoring scale, three random fields of lung tissue were scored for the following in a blinded manner: 1=absence of cellular sloughing and necrosis, 2-Uncommon solitary cell sloughing and necrosis (1-2 foci/field), 3=multifocal (3+foci) cellular sloughing and necrosis with uncommon septal wall hyalinization, or 4-multifocal (>75% of field) cellular sloughing and necrosis with common and/or prominent hyaline membranes. The scores for the three fields per mouse were averaged to get a final DAD score per mouse. The microscope images were generated using an Olympus Bx43 light microscope and CellSense Entry v3.1 software.

### Biocontainment and Biosafety

Studies were approved by the UNC Institutional Biosafety Committee approved by animal and experimental protocols in the Baric laboratory. All work described here was performed with approved standard operating procedures for SARS-COV-2 in a biosafety level 3 (BSL-3) facility conforming to requirements recommended in the Microbiological and Biomedical Laboratories, by the U.S. Department of Health and Human Service, the U.S. Public Health Service, and the U.S. Center for Disease Control and Prevention (CDC), and the National Institutes of Health (NIH).

### Weblogo

As the interface in the SARS-COV-2/DH1047 complex was better resolved (16), we used this complex for visualizing epitope details and analyzing extent of conservation of the epitope residues in different sarbecoviruses. The Weblogo tool from UC Berkeley was used to generate the consensus epitopes of the DH1047 HCDR3 and LCDR3 epitopes. weblogo.berkeley.edu/logo.cgi. The following 23 Sarbecovirus genomes were used for the Weblogo analysis: KT444582, AY278554, AY278741, AY515512, AY525636, KC881005, KC881006, KF367457, MK211376, MN908947, MN996532, MG772933, MG772934, MK211374, DQ412042, DQ648856, MK211378, DQ022305, DQ412043, DQ648857, DQ071615, MK211375, MK211377.

Statistics: All statistical analyses were performed using GraphPad Prism 9.

### REFERENCES

1. J. S. Peiris et al., Coronavirus as a possible cause of severe acute respiratory syndrome. Lancet 361, 1319-1325 (2003).
2. J. D. Cherry, P. Krogstad, SARS: The First Pandemic of the 21st Century. Pediatric Research 56, 1-5 (2004).
3. P. Zhou et al., A pneumonia outbreak associated with a new coronavirus of probable bat origin. Nature 579, 270-273 (2020).
4. W. Li et al., Bats Are Natural Reservoirs of SARS-Like Coronaviruses. Science 310, 676-679 (2005).
5. B. Hu et al., Discovery of a rich gene pool of bat SARS-related coronaviruses provides new insights into the origin of SARS coronavirus. PLOS Pathog 13, e1006698 (2017).
6. V. D. Menachery et al., A SARS-like cluster of circulating bat coronaviruses shows potential for human emergence. Nat Med 21, 1508-1513 (2015).
7. V. D. Menachery et al., SARS-like WIV1-COV poised for human emergence. Proc Natl Acad Sci USA 113, 3048-3053 (2016).
8. D. R. Martinez et al., Chimeric spike mRNA vaccines protect against Sarbecovirus challenge in mice. Science, (2021).
9. A. C. Walls et al., Elicitation of broadly protective sarbecovirus immunity by receptor-binding domain nanoparticle vaccines, bioRxiv, (2021).
10. K. O. Saunders et al., Neutralizing antibody vaccine for pandemic and pre-emergent coronaviruses. Nature, (2021).
11. L. Wang et al., Antibodies with potent and broad neutralizing activity against antigenically diverse and highly transmissible SARS-COV-2 variants, bioRxiv, (2021).
12. P. Wang et al., Antibody resistance of SARS-COV-2 variants B.1.351 and B.1.1.7. Nature, (2021).
13. R. E. Chen et al., Resistance of SARS-COV-2 variants to neutralization by monoclonal and serum-derived polyclonal antibodies. Nat Med, (2021).
14. D. Li, G. D. Sempowski, K. O. Saunders, P. Acharya, B. F. Haynes, SARS-CoV-2 Neutralizing Antibodies for COVID-19 Prevention and Treatment. Annual Review of Medicine 73, null (2022).
15. C. G. Rappazzo et al., Broad and potent activity against SARS-like viruses by an engineered human monoclonal antibody. Science 371, 823-829 (2021).
16. D. Li et al., In vitro and in vivo functions of SARS-COV-2 infection-enhancing and neutralizing antibodies. Cell 184, 4203-4219.e4232 (2021).
17. J. R. Whittle et al., Broadly neutralizing human antibody that recognizes the receptor-binding pocket of influenza virus hemagglutinin. Proc Natl Acad Sci USA 108, 14216-14221 (2011).
18. S. R. Leist, A. Schäfer, D. R. Martinez, Cell and animal models of SARS-COV-2 pathogenesis and immunity. Dis Model Mech 13, (2020).
19. D. Planas et al., Sensitivity of infectious SARS-COV-2 B.1.1.7 and B.1.351 variants to neutralizing antibodies. Nature Medicine, (2021).
20. C. O. Barnes et al., SARS-COV-2 neutralizing antibody structures inform therapeutic strategies. Nature 588, 682-687 (2020).
21. Z. Lv et al., Structural basis for neutralization of SARS-COV-2 and SARS-COV by a potent therapeutic antibody. Science 369, 1505-1509 (2020).
22. L. Piccoli et al., Mapping Neutralizing and Immunodominant Sites on the SARS-COV-2 Spike Receptor-Binding Domain by Structure-Guided High-Resolution Serology. Cell 183, 1024-1042.e1021 (2020).
23. H. Liu et al., Cross-neutralization of a SARS-COV-2 antibody to a functionally conserved site is mediated by avidity, bioRxiv, (2020).
24. D. Zhou et al., Structural basis for the neutralization of SARS-COV-2 by an antibody from a convalescent patient. Nat Struct Mol Biol 27, 950-958 (2020).
25. N. G. Davies et al., Estimated transmissibility and impact of SARS-COV-2 lineage B.1.1.7 in England. Science 372, eabg3055 (2021).
26. N. G. Davies et al., Increased mortality in community-tested cases of SARS-CoV-2 lineage B.1.1.7. Nature, (2021).
27. C. Liu et al., Reduced neutralization of SARS-COV-2 B.1.617 by vaccine and convalescent serum. Cell 184, 4220-4236.e4213 (2021).
28. J. Sadoff et al., Safety and Efficacy of Single-Dose Ad26.COV2.S Vaccine against Covid-19. New England Journal of Medicine, (2021).
29. S. A. Madhi et al., Efficacy of the ChAd0×1 nCOV-19 Covid-19 Vaccine against the B.1.351 Variant. New England Journal of Medicine, (2021).
30. Z. Wang et al., mRNA vaccine-elicited antibodies to SARS-COV-2 and circulating variants. Nature, (2021).
31. C. Wang et al., A conserved immunogenic and vulnerable site on the coronavirus spike protein delineated by cross-reactive monoclonal antibodies. Nature Communications 12, 1715 (2021).
32. P. Zhou et al., A protective broadly cross-reactive human antibody defines a conserved site of vulnerability on beta-coronavirus spikes, bioRxiv, (2021).
33. M. M. Sauer et al., Structural basis for broad coronavirus neutralization. bioRxiv, (2020).
34. M. F. Jennewein et al., Isolation and Characterization of Cross-Neutralizing Coronavirus Antibodies from COVID-19+Subjects, bioRxiv, (2021).
35. M. A. Tortorici et al., Structural basis for broad sarbecovirus neutralization by a human monoclonal antibody, bioRxiv, (2021).
36. C. A. Jette et al., Broad cross-reactivity across sarbecoviruses exhibited by a subset of COVID-19 donor-derived neutralizing antibodies, bioRxiv, 2021.2004.2023.441195 (2021).
37. C. W. Tan et al., Pan-Sarbecovirus Neutralizing Antibodies in BNT162b2-Immunized SARS-COV-1 Survivors. N Engl J Med, (2021).
38. D. R. Martinez et al., Prevention and therapy of SARS-COV-2 and the B.1.351 variant in mice. Cell Reports.
39. S. Ravichandran et al., Antibody signature induced by SARS-COV-2 spike protein immunogens in rabbits. Sci Transl Med 12, (2020).
40. J. Tang et al., Antibody affinity maturation and plasma IgA associate with clinical outcome in hospitalized COVID-19 patients. Nat Commun 12, 1221 (2021).
41. D. Wrapp et al., Cryo-EM structure of the 2019-nCOV spike in the prefusion conformation. Science 367, 1260-1263 (2020).
42. R. Henderson et al., Controlling the SARS-COV-2 spike glycoprotein conformation. Nat Struct Mol Biol, (2020).
43. R. J. Edwards et al., Cold sensitivity of the SARS-COV-2 spike ectodomain. Nature Structural & Molecular Biology 28, 128-131 (2021).
44. W. B. Williams et al., Fab-dimerized glycan-reactive antibodies are a structural category of natural antibodies. Cell 184, 2955-2972.e2925 (2021).
45. A. Punjani, J. L. Rubinstein, D. J. Fleet, M. A. Brubaker, cryoSPARC: algorithms for rapid unsupervised cryo-EM structure determination. Nature Methods 14, 290-296 (2017).
46. D. Liebschner et al., Macromolecular structure determination using X-rays, neutrons and electrons: recent developments in Phenix. Acta Crystallographica Section D Structural Biology 75, 861-877 (2019).
47. P. V. Afonine et al., Real-space refinement inPHENIXfor cryo-EM and crystallography. Acta Crystallographica Section D Structural Biology 74, 531-544 (2018).
48. P. Emsley, B. Lohkamp, W. G. Scott, K. Cowtan, Features and development of Coot. Acta Crystallographica Section D Biological Crystallography 66, 486-501 (2010).
49. L. Schrodinger, The PyMOL Molecular Graphics System. (2015).
50. E. F. Pettersen et al., UCSF Chimera?A visualization system for exploratory research and analysis. Journal of Computational Chemistry 25, 1605-1612 (2004).
51. T. D. Goddard et al., UCSF ChimeraX: Meeting modern challenges in visualization and analysis. Protein Science 27, 14-25 (2018).
52. T. I. Croll, ISOLDE: a physically realistic environment for model building into low-resolution electron-density maps. Acta Crystallogr D Struct Biol 74, 519-530 (2018).
53. S. R. Leist et al., A Mouse-Adapted SARS-COV-2 Induces Acute Lung Injury and Mortality in Standard Laboratory Mice. Cell 183, 1070-1085.e1012 (2020).
54. A. Roberts et al., A mouse-adapted SARS-coronavirus causes disease and mortality in BALB/c mice. PLOS Pathog 3, e5 (2007).
55. T. P. Sheahan et al., Comparative therapeutic efficacy of remdesivir and combination lopinavir, ritonavir, and interferon beta against MERS-COV. Nature Communications 11, 222 (2020).
56. M. E. Schmidt et al., Memory CD8 T cells mediate severe immunopathology following respiratory syncytial virus infection. PLOS Pathog 14, e1006810 (2018).

1. A recombinant coronavirus monoclonal antibody, or an antigen binding fragment thereof, which binds to coronavirus spike protein and comprises a variable heavy (VH) domain and a variable light (VL) domain that have amino acid sequences that have an overall 80% sequence identity to the VH and VL domains of an antibody DH1041, DH1042, DH1043, DH1044, DH1045, DH1046, DH1047, DH1048, DH1049, DH1050.2, DH1051, DH1050.1, DH1052, DH1053, DH1054, DH1055, DH1056, DH1057, DH1058, DH1073, DH1221, DH1222, DH1223, DH1224, DH1225, DH1226, DH1235 or DH1236listed in Tables 3 6, 10 of 11, or wherein the VH domain and VL domain each have at least 80% sequence identity to the VH and VL domains, respectively, of an antibody DH1041, DH1042, DH1043, DH1044, DH1045, DH1046, DH1047, DH1048, DH1049, DH1050.2, DH1051, DH1050.1, DH1052, DH1053, DH1054, DH1055, DH1056, DH1057, DH1058, DH1073, DH1221, DH1222, DH1223, DH1224, DH1225, DH1226, DH1235 or DH1236.

2. A recombinant coronavirus monoclonal antibody, or an antigen binding fragment thereof, which binds coronavirus spike protein, which comprises:
a. Vh domain CDRH1-3 regions from an antibody DH1041, DH1042, DH1043, DH1044, DH1045, DH1046, DH1047, DH1048, DH1049, DH1050.2, DH1051, DH1050.1, DH1052, DH1053, DH1054, DH1055, DH1056, DH1057, DH1058, DH1073, DH1221, DH1222, DH1223, DH1224, DH1225, DH1226, DH1235 or DH1236; and/or
b. VI domain CDRL1-3 regions from an antibody DH1041, DH1042, DH1043, DH1044, DH1045, DH1046, DH1047, DH1048, DH1049, DH1050.2, DH1051, DH1050.1, DH1052, DH1053, DH1054, DH1055, DH1056, DH1057, DH1058, DH1073, DH1221, DH1222, DH1223, DH1224, DH1225, DH1226, DH1235 or DH1236listed in Table 4, wherein the Vh and VI are from the same antibody; and
c, wherein the framework of the variable heavy (Vh) domain comprises amino acid sequences that have at least 90% sequence identity to the V gene, D gene and J gene of the Vh gene of the corresponding antibody from which the CDRs are derived and wherein the framework of the variable light (VI) domain comprises amino acid sequences that have at least 90% sequence identity to the V and J genes of the VI gene from the corresponding antibody from which the CDRs are derived.

3. The recombinant antibody or the antigen binding fragment thereof of claim 2, wherein the antibody is DH1047 and the framework of the variable heavy (Vh) domain comprises amino acid sequences derived from human IGHV1-46 and IGHJ4 Ig genes and wherein the framework of the variable light (VI) domain comprises amino acid sequences derived from IGKV4-1 and IGKJI human IgG genes.

4. The recombinant antibody or the antigen binding fragment thereof according to claim 2, wherein the antibody is DH1073 and the framework of the variable heavy (Vh) domain comprises amino acid sequences derived from human IGHV1-46 and IGHJ6 Ig genes and wherein the framework of the variable light (VI) domain comprises amino acid sequences derived from IGKV3-11 and IGKJ1 human IgG genes.

5. The recombinant antibody or the antigen binding fragment thereof [of] according to claim 2, wherein the antibody is DH1042 and the framework of the variable heavy (Vh) domain comprises amino acid sequences derived from IGH1-69 and IGHJ6 human Ig genes and wherein the framework of the variable light (VI) domain comprises amino acid sequences derived from IGKV1-39 and IGKJ2 human IgG genes.

6. The recombinant antibody or the antigen binding fragment thereof according to claim 1, wherein the antibody or antigen binding fragment thereof, comprises a heavy chain comprising at least one CDRH1, at least one CDRH2 and at least one CDRH3 and a light chain comprising at least one CDRL1, at least one CDRL2 and at least one CDRL3, wherein at least one CDR, comprises an amino acid sequence according to any of paired VHH712384 and VI K711897 (DH1047), Vh DH1073 VH and VI DH1073 VK (DH1073), Vh DH1235 VH and VI DH1235 VK (DH1235), Vh H026103 and VI K023879 (DH1042), VH H026124 and VL L024055 (DH1041), VH H026116 and VL K023888 (DH1043), or a functional sequence variant thereof having at least 70%, at least 75%, at least 80%, at least 85%, at least 88%, at least 90%, at least 92%, at least 95%, at least 96%, at least 97%, at least 98% or at least 99% sequence identity.

7. The recombinant antibody or the antigen binding fragment thereof, according to claim 6, wherein the antibody or antigen binding fragment thereof, comprises paired VHH712384 and VI K711897 (DH1047), VhDH1073 VH and VI DH1073 VK (DH1073), Vh DH1235 VH and VI DH1235 VK (DH1235), VH H026103 and VI K023879 (DH1042), VH H026124 and VL L024055 (DH1041), VH H026116 and VL K023888 (DH1043) or a functional sequence variant thereof having at least 70%, at least 75%, at least 80%, at least 85%, at least 88%, at least 90%, at least 92%, at least 95%, at least 96%, at least 97%, at least 98% or at least 99% sequence identity.

8. The recombinant antibody, or the antigen binding fragment thereof, according to claim 1, wherein the antibody, or antigen binding fragment thereof binds to coronavirus domain RBD, NTD, or S2.

9. The antibody, or the antigen binding fragment thereof, according to claim 2, wherein the antibody, or antigen binding fragment thereof binds RBD.

10. The recombinant antibody, or the antigen-binding fragment thereof, according to claim 1, wherein the antibody, or antigen-binding fragment thereof, comprises an Fc moiety.

11. The recombinant antibody, or the antigen binding fragment thereof, according to claim 1, wherein the antibody, or the antigen binding fragment thereof, is a purified antibody IgG antibody, a multivalent antibody, multispecific antibody, a single chain antibody, Fab, Fab′, F(ab′)2, Fv or scFv.

12. The recombinant antibody, or the antigen-binding fragment thereof, according to claim 1, for use as a medicament.

13. A nucleic acid molecule comprising a polynucleotide encoding the antibody, or the antigen-binding fragment thereof, according to claim 1.

14. The nucleic acid molecule according to claim 13, wherein the polynucleotide sequence comprises a nucleic acid sequence pH026124 LS,v2, pL024055,v2, pH026103 LS,v2, pK023879,v2, pH026116 LS,v2, pK023888,v2, pH026077 LS,v2, pL024038,v2, pH026066 LS,v2, pL024032 L024042,v2, pH026204 LS,v2, pK023942,v2, pH712384 LS,v2, pK711897,v2, pL024008,v2, pH711733 G1.4 Å, pK711421,v2, pH026107,LS,v2, pK023882,v2, pH712151 LS,v2, pL711550,v2, pH025976 LS,v2, pK023811,v2, pH025988 LS,v2, pK023817,v2, pH025934 G1.4 Å, pK023788,v2, pH711725 G1.4 Å, pK711414,v2, DH1050.1 IgG1 LS ab026016, DH1051 IgG1 LS Ab026013, DH1050.1 IgL Ab026016, DH1051 IgL Ab026013, pH712564 LS,v2, pH712581 LS,v2, pH712587 LS,v2, pH712589 LS,v2, pH712593 H712600 LS,v2, pH712611 LS,v2, pK712022,v2, pK712032,v2, pK712037,v2, pK712038,v2, pK712041,v2, pK712052,v2, H026116 DNA Sequence, H026116 Expressed mRNA Sequence, K023888 DNA Sequence, K023888 Expressed mRNA Sequence, H026124 DNA Sequence, H026124 Expressed mRNA Sequence, L024055 DNA Sequence, L024055 Expressed mRNASequence, Duke P3 IgG1 LS IVT Plasmid Sequence, Duke P3 1gL IVT Plasmid Sequence, Duke P3 IgK IVT Plasmid Sequence, pH712189 LS,v2, pK711798,v2, pK711798,v2, DH1073 VH, DH1073 VK, DH1235 VH, DH1235 VK; or a functional sequence variant thereof having at least 70%, at least 75%, at least 80%, at least 85%, at least 88%, at least 90%, at least 92%, at least 95%, at least 96%, at least 97%, at least 98% or at least 99% sequence identity.

15. The nucleic acid of claim 13 wherein the nucleic acid is a ribonucleic acid (RNA).

16. The nucleic acid of claim 15, wherein the RNA is suitable for use and delivery as a therapeutic mRNA.

17. A vector comprising the nucleic acid molecule according to claim 13.

18. A cell expressing the antibody, or the antigen binding fragment thereof, according to claim 1.

19. A pharmaceutical composition comprising the antibody, or the antigen binding fragment thereof, according to claim 1, and a pharmaceutically acceptable carrier.

20. A pharmaceutical composition comprising the antibody, or the antigen binding fragment thereof, wherein the antibody, or the antigen binding fragment thereof, comprise a Vh and VI sequence of claim 1, wherein the Vh and VI sequences form a multivalent or multispecific antibody.

21. A pharmaceutical composition comprising at least one RBD binding antibody and at least one NTD binding antibody of claim 8.

22. A pharmaceutical composition comprising two RBD binding antibodies or antigen binding fragments thereof, wherein the two antibodies or antigen binding fragments thereof have non-overlapping epitopes.

23. The pharmaceutical composition according to claim 20 further comprising a pharmaceutically acceptable excipient, diluent or carrier.

24. A method of treating or preventing coronavirus infection in a subject in need thereof, comprising administering the recombinant antibody of claim 1 in an amount suitable to effect treatment or prevention of coronavirus infection.

25. The method of claim 24, wherein the antibody is administered prior to coronavirus exposure or at the same time as coronavirus exposure.

26. A method of treating or preventing coronavirus infection comprising administering a therapeutic or prophylactic amount of a composition comprising an antibody or antigen binding fragment thereof comprising a Vh and VI sequence of claim 1, wherein the Vh and VI sequences are comprised in a bi- or tri-specific antibody format or in a multivalent format.
